SEQUENCE LISTING (1 sequence)

SEQ ID NO: 1 - protein, 218 aa, Homo sapiens
IVGGRRARPHAWPFMVSLQLRGGHFCGATLIAPNFVMSAAHCVANVNVRAVRVVLGAHNLSRREPTRQVFAVQRIFEDGYDPVNLLNDIVILQLNGSATINANVQVAQLPAQGRRLGNGVQCLAMGWGLLGRNRGIASVLQELNVTVVTSLCRRSNVCTLVRGRQAGVCFGDSGSPLVCNGLIHGIASFVRGGCASGLYPDAFAPVAQFVNWIDSIIQ

(19) United States
(12) Patent Application Publication
(10) Pub. No.: US 2010/0003709 A1
(43) Pub. Date: Jan. 7, 2010

(54) Crystal Structure of Enzyme and Uses Thereof

(75) Inventors: Peter Reinemer, Wuppertal (DE); Heike Gielen-Haertwig, Monheim (DE); Ulrich Rosentreter, Binnen (DE); Volkhart Li, Velbert (DE); Axel Harrenga, Wuppertal (DE); Dietmar Schomburg, Erftstadt-Lechenich (DE); Karsten Niefind, Koln (DE); Guido Hansen, Victoria (AU)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bayer HealthCare AG, Leverkusen (DE)

(21) Appl. No.: 11/793,367

(22) PCT Filed: Dec. 13, 2005
(86) PCT No.: PCT/EP2005/013370

§ 371 Date: Jun. 3, 2008

(30) Foreign Application Priority Data
Dec. 16, 2004 (EP) 04029768.1

(51) Int. Cl. C12Q 1/37; C12N 9/66; G06G 7/48; C07D 239/22

(52) U.S. Cl. 435/23; 435/218; 703/11; 544/316

(57) ABSTRACT

This invention relates to crystallised human neutrophil elastase and the use of its three-dimensional structure to design modulators for human neutrophil elastase.

### FIELD OF THE INVENTION

This invention relates to crystallised human neutrophil elastase and the use of its three-dimensional structure to design modulators for human neutrophil elastase.

### BACKGROUND OF THE INVENTION

Proteins such as enzymes involved in physiological and pathological processes are important targets in the development of pharmaceutical active ingredients and treatments. Knowledge of the three-dimensional (tertiary) structure of proteins allows the rational design of mimics or modulators of such proteins. By searching structural databases using structural parameters derived from the protein of interest, it is possible to select molecular structures that may mimic or interact with these parameters. It is then possible to synthesize the selected molecular structure and test its activity. Alternatively, the structural parameters derived from the protein of interest may be used to design and synthesize a mimic or modulator with the desired activity. Such mimics or modulators may be useful as therapeutic agents for treating certain diseases. Knowledge of the three-dimensional structure of a protein alone or in complex with modulators of that protein is essential for the rational design of other mimics or modulators of that protein. Lack of structural knowledge is a barrier to the development of new mimics or modulators that may have extremely useful pharmaceutical properties.

Chronic Obstructive Pulmonary Disease (COPD) is a group of slowly progressive disorders characterized by airflow obstruction caused by emphysema and chronic bronchitis. Generally beginning in the fourth to fifth decade of life, COPD has an insidious onset. But over the next several years, mild shortness of breath that often goes unnoticed escalates to more serious symptoms that cannot be ignored: cough, copious and sometimes purulent sputum production, wheezing, and difficulty breathing (dyspnea). Advanced COPD typically causes significant debilitation and increases the risk of complications such as pneumonia or cardiac abnormalities. COPD primarily is a disease of cigarette smokers, with smoking responsible for 90% of all cases of COPD and 80-90% of COPD deaths. COPD is a major medical problem and worldwide is the sixth leading cause of death, affecting 4-6% of people older than 45 years. Although airflow obstruction may be partially and temporarily reversible, there is no cure for COPD. Consequently, the goal of treatment is to improve quality of life by relieving symptoms, preventing acute exacerbations, and slowing progressive deterioration in lung function. Existing pharmacotherapy, which has changed little over the past two to three decades, mainly consists of the use of bronchodilators to open blocked airways and, in certain situations, the use of corticosteroids to reduce lung inflammation (Barnes P J, 2000, N. Engl. J. Med. 343: 269-280).

Chronic lung inflammation induced by cigarette smoke or other irritants is the driving force behind the development of the disease. Major pathophysiological changes associated with COPD are emphysema, which is characterised by abnormal and permanent alveolar enlargement, and chronic bronchitis, which is characterized by persistent and recurring inflammation of the bronchi clinically defined by a chronic cough and mucus production. The underlying mechanisms involve immune cells releasing various chemokines during inflammatory responses in the lung. Thereby, neutrophils are attracted to lung connective tissue and airway lumen and as they accumulate, also attract alveolar macrophages. In a vital immune function known as phagocytosis both cell types together secrete protease cocktails, largely consisting of neutrophil released elastase and proteinase 3 and macrophage released matrix metalloproteases intended for the destruction of invading bacteria, dead and damaged cells and other microorganisms. The body in turn releases antiproteolytic proteins to protect the delicate, nonregenerative architecture of the lung from injury by proteolytic activity. The most important antiproteolytic agent is α₁-antitrypsin which neutralizes neutrophil elastase. The prevailing theory of pathogenesis of emphysema is that the protease-antiprotease balance shifts in favor of proteolysis, resulting in unchecked elastase activity that dissolves elastin lining the alveolar walls (Gadek J E et al, 1981, J. Clin. Invest. 68: 889-898; Werb Z et al, 1982, J. Invest. Dermatol. 79: 154-159; Janoff A, 1985, Am. Rev. Respir. Dis. 132: 417-433; Barnes P J, 2000, N. Engl. J. Med. 343: 269-280). This tissue breakdown causes air sac merging, leading to bronchiole collapse, decreased lung elasticity, airway obstruction, and impaired expiration (Barnes P J, 2000, N. Engl. J. Med. 343: 269-280). In addition, frequent or prolonged lung inflammation can lead to bronchial remodelling ultimately leading to the formation of airway lesions. By obstructing airflow and increasing mucus production, these lesions contribute both to the chronic productive cough that typifies chronic bronchitis and, possibly, to airway hyperresponsiveness.

The pivotal importance of neutrophil elastase in the onset and progression of the disease qualifies the enzyme as an important target for the development of a therapeutic intervention of COPD and a potent, selective and orally active inhibitor of neutrophil elastase is believed to inhibit the progression of the long-term lung function decline in COPD patients by inhibition of neutrophil induced upregulation of pro-inflammatory chemokines such as interleukin-8 and by inhibition of elastase mediated lung tissue destruction (Gadek J E et al, 1981, J. Clin. Invest. 68: 889-898; Werb Z et al, 1982, J. Invest. Dermatol. 79: 154-159; Janoff A, 1985, Am. Rev. Respir. Dis. 132: 417-433; Ohbayashi H, 2002, Expert Opin. Invest. Drugs 11: 965-980).

Human Neutrophil Elastase (HNE; EC 3.4.21.37, also named human leukocyte elastase or PMN elastase) is a serine protease from the trypsin family and is one of several proteolytic enzymes contained in the azurophil granules of human neutrophils. HNE is a strongly basic glycoprotein of a molecular weight of about 30 kDa (Barret A J, 1981, Meth, Enzymol. 80: 581-588). The sequence of HNE was initially established by combined methods of peptide sequencing (Sinha S et al, 1987, Proc. Natl. Acad. Sci. USA 84: 2228-2232) and X-ray-crystallography (Bode W et al, 1986, EMBO J. 5: 2453-2458). HNE consists of a single basic polypeptide chain comprising 218 amino acid residues, four intra-chain disulfide links, and two asparagine-linked carbohydrate side chains, and is in fact synthesized as a series of isoenzymes each containing different amounts of carbohydrate (Sinha S et al, 1987, Proc. Natl. Acad. Sci. USA 84: 2228-2232). Subsequently, cDNA analysis (Farle D et al, 1988, Biol. Chem. Hoppe Seyler 369: Suppl. 3-7; Takahashi H et al, 1988, J. Biol. Chem. 263: 14739-14747) revealed a 20 amino acid extension at the C-terminus of the protein, which is probably removed during post-translational modification. HNE exhibits a very narrow substrate specificity and cleaves preferentially Val-X bonds and to a lesser extent Ala-X bonds, which are preferred by pancreatic elastase (Harris J L et al, 2000, Proc. Natl. Acad. Sci USA 97: 7754-7759).

The importance of HNE as a pathogenic agent in various diseases has stimulated the search for potent inhibitors. Despite of its high medicinal relevance and many biochemically characterized inhibitors (Powers J C, 1983, Am. Rev. Respir. Dis. 127: 54-589; Leung D et al, 2000, J. Med. Chem. 44: 1268-1285; Ohbayashi H, 2002, Expert Opin. Invest. Drugs 11: 965-980) only a limited number of crystal structures of HNE have been published. Four complex structures with small molecule inhibitors, all forming covalent bounds to the active site serine residue of HNE, have been published (Wei A Z et al, 1988, FEBS Lett., 234: 367-373; Navia M A et al, 1989, Proc. Natl. Acad. Sci USA 86: 7-11; Cregge R J et al, 1998, J. Med. Chem. 41: 2461-2480; MacDonald R J et al, 2002, J. Med. Chem. 45, 3878-3890). In addition, one protein-polypeptide complex of HNE with a 56 amino acid protease inhibitor has been determined (Bode W et al, 1986, EMBO J. 5: 2453-2458). To rationalize structure-activity relationships of various classes of inhibitors often complexes of porcine pancreatic elastase (PPE) and inhibitors have been used (Veale C A et al, 1995, J. Med. Chem. 38, 98-108; Cregge R J et al, 1998, J. Med. Chem. 41: 2461-2480; Ohmoto K et al, 2001, J. Med. Chem. 44: 1268-1285). This approach may be problematic because of the relatively low overall sequence homology between human neutrophil elastase and porcine pancreatic elastase (40%) and a number of amino acids exchanges in the active sites of HNE and PPE.

The previously described structures provide information about the overall structure of the enzyme and complexes with suicide peptidomimetic inhibitors, but to date, no structural information is known about the way in which non-covalently binding, non-peptidic inhibitors bind to the enzyme. Further, it has not been possible to obtain crystals from human neutrophil elastase in an un-inhibited form.

The X-ray crystal structure of human neutrophil elastase in complex with a synthetic pyrimidinone inhibitor has been determined. An X-ray crystal structure of an un-inhibited from of human neutrophil elastase, where the enzyme is detached from a/its ligand(s), has also been produced. These structures not only provide important structural information on the free enzyme of human neutrophil elastase which is detached of an/its inhibitory ligand(s) and on a novel class of inhibitors and their mode of inhibition of human neutrophil elastase but will also assist the design of more potent and specific inhibitors to treat the many diseases where human neutrophil elastase plays a role.

### SUMMARY OF THE INVENTION

The present invention relates to the previously unknown three-dimensional structure of the un-inhibited form of human neutrophil elastase, where said enzyme is detached from a/its ligand(s). As described herein, the applicants have overcome the difficulties encountered by others and have produced crystals of an un-inhibited form of human neutrophil elastase that are of sufficient quality to determine the three-dimensional structure of the protein by X-ray diffraction methods. In addition, the applicants have determined the three-dimensional crystal structure of human neutrophil elastase in complex with a synthetic pyrimidinone inhibitor. There is a clear need for this structural information to enable identification and structure-based design of new human neutrophil elastase modulators (particularly inhibitors) for the treatment of various diseases or conditions and in particular respiratory diseases such as asthma and COPD. The methods described herein allow the determination of the three-dimensional structures of un-inhibited human neutrophil elastase, as well as of complexes of human neutrophil elastase with numerous inhibitors of interest to aid in the rational design of modulators that will treat respiratory diseases.

### BRIEF DESCRIPTION OF THE DRAWINGS

FIG. 1a is a schematic representation of the structure of un-inhibited human neutrophil elastase. FIG. 1b is a schematic representation of the structure of human neutrophil elastase in complex with a synthetic pyrimidinone inhibitor.

FIG. 2a is a schematic representation of human neutrophil elastase in complex with a synthetic pyrimidinone inhibitor showing the inhibitor occupying the active site binding pocket. FIG. 2b is a schematic representation of human neutrophil elastase in complex with a synthetic pyrimidinone inhibitor showing the inhibitor occupying the active site binding pocket in surface representation and with enzyme subsites labelled according to the nomenclature of Schechter and Berger (Schechter I & Berger A, 1967, Biochem. Biophys. Res. Commun. 27: 157-162). FIG. 2c is a schematic surface representation of the pyrimidinone inhibitor bound to the subsites of the human neutrophil elastase active site binding pocket.

FIG. 3a is a schematic representation comparing the active site binding pocket of human neutrophil elastase in its un-inhibited form and in its pyrimidinone-inhibited form. FIG. 3b is a schematic representation showing the active site binding pocket of pyrimidinone-inhibited human neutrophil elastase in a surface representation with the superimposed schematic representations of the un-inhibited form of human neutrophil elastase.

FIG. 4 is showing a synthesis scheme for the synthesis of inhibitor I.

### DETAILED DESCRIPTION OF THE INVENTION

The terms “apo”, “un-complexed”, or “un-inhibited” as used herein are taken to mean any protein (or named protein) that is detached from a/its ligand(s) and/or prosthetic group(s).

The term “active site” as used herein is taken to include any site (e.g. specific groups) within a molecule (and associated metal ions and/or hydration molecules) where specific activity is undergone. Such activity could include binding of a ligand to the site, catalysis of the molecule's substrates by the site, recognition of a ligand by the site, etc.

The term “active site subsite” as used herein is taken to include any particular fraction of the active site within a molecule according to the above given definition. In particular, subsite nomenclature as defined by Schechter and Berger (Schechter I & Berger A, 1967, Biochem. Biophys. Res. Commun. 27: 157-162) is used to identify such subsites.

The term “buffer” as used herein is taken to include any solution containing a weak acid and a conjugate base of this acid (or, less commonly, a weak base and its conjugate acid). Thus, a “buffer” as used herein resists change in its pH level when an acid or a base is added to it, because the acid neutralises an added base (or, less commonly, the base neutralises an added acid).

The term “precipitant” as used herein is taken to include any substance that, when added to solution (usually of macromolecules), causes a precipitate to form or crystals to grow.

The terms “complex”, “complexed”, or “inhibited” as used herein are taken to mean a protein with ligand(s) bound and may be formed before, during or after protein crystallization.

The term “soaking” as used herein is taken to mean the addition of a solution containing a (usually) small molecule (e.g. inhibitor) to crystals of a protein to form a protein-ligand complex.

The term “co-crystallisation” as used herein is taken to mean crystallisation of a pre-formed protein/small molecule complex.

The term “atomic coordinates” as used herein is taken to refer to mathematical coordinates corresponding to the positions of every atom derived from mathematical equations related to the diffraction patterns obtained from a monochromatic beam of X-rays illuminating a crystal. The diffraction data are used to calculate an electron density map of the repeating unit of the crystal. The electron density maps are used to establish the positions of the individual atoms within the unit cell of the crystal. Those of skill in the art understand that a set of atomic coordinates determined by X-ray crystallography is not without standard error or experimental variation.

The term “unit cell” as used herein is taken to refer to the basic building block from which the entire volume of a crystal may be constructed.

The term “asymmetric unit” as used herein is taken to refer to the minimal ensemble of atoms comprising the repeating unit of the unit cell.

The term “space group” as used herein is taken to refer to the arrangement of symmetry elements within a unit cell.

The term “molecular replacement” as used herein is taken to refer to a method that involves generating a preliminary model of a crystal whose atomic coordinates are not known, by orienting and positioning a related ensemble of atoms (preferably a molecule) whose atomic coordinates are known. Phase information required for electron density calculation are then calculated from this model and combined with observed amplitudes to give an approximate Fourier synthesis of the structure whose coordinates are unknown.

The term “main chain phi angle” or “phi angle” as used herein is taken to refer to the rotation angle around the N—Cα bond of an amino acid residue. This rotation angle is described by the torsion angle C_{(residue n-1)}—N_{(residue n)}—Cα_{(residue n)}-C_{(residue n)}.

The term “main chain psi angle” or “psi angle” as used herein is taken to refer to the rotation angle around the Cα-C bond of an amino acid residue. This rotation angle is described by the torsion angle N_{(residue n)}—Cα_{(residue n)}-C_{(residue n)}—N_{(residue n+1)}.

Torsion angles of the general definition Atom_{front}-Atom_{central1}-Atom_{central2}-Atom_{back} as given herein are defined as positive values if Atom_{front} has to rotated clockwise around the bond between Atom_{central1}-Atom_{central2} to superimpose with Atom_{back}. Accordingly, they are defined as negative values if Atom_{front} has to rotated counter-clockwise around the bond between Atom_{central1}-Atom_{central2} to superimpose with Atom_{back}.

The term “centroid” as used herein is taken to refer to the x,y,z coordinates in orthogonal Angstrom space of the intersection point of all lines connecting opposite atoms in a 6-membered ring; the term “centroid distance” as used herein is taken to refer to the distance between the centroid of a given ring system to any other atom within the protein structure as calculated from the difference of their x,y,z coordinates in orthogonal Angstrom space.

This invention relates to crystals of human neutrophil elastase and the use of the three-dimensional structure to design modulators (preferably inhibitors) of human neutrophil elastase. It further relates to crystals of human neutrophil elastase, complexed or un-complexed as described, of sufficient quality to determine the three-dimensional (tertiary) structure of the polypeptide by X-ray diffraction methods.

According to a first aspect of the invention, the applicants provide two crystalline forms of a polypeptide comprising human neutrophil elastase. One crystalline form is obtained when we crystallise human neutrophil elastase in an un-complexed from. The second crystalline form is obtained when we crystallise human neutrophil elastase in the presence of a synthetic pyrimidinone inhibitor. In one embodiment, the first crystalline form has the space group P4₁2₁2. In another embodiment, the first crystalline form has the unit cell dimensions a=b=123.22, c=68.82 Å, α=β=γ=90°. In another embodiment the second crystalline form has the space group P6₃. In another embodiment the second crystalline form has the unit cell dimensions a=b=73.73, c=70.66 Å, α=β=90°, γ=120°. In another embodiment, these crystalline forms are described by three-dimensional sets of x,y,z-coordinates (Tables 1 and 2) for each atom in the ensemble representing the unique repeating motif in the crystal. Table 1 contains the coordinates for two independent un-complexed human neutrophil elastase molecules in the asymmetric unit in the first crystalline form; Table 2 contains the coordinates for the human neutrophil elastase complex in the second crystalline form. In another embodiment, these crystalline forms contain a numerical definition of a binding site, approximated by the set of all residues within a 5 Å contact distance from any atom in either inhibitor. Accordingly, the binding site is defined by the x,y,z-coordinates of atoms in the set given by the list His57, Tyr94, Pro98, Leu99B, Leu100, Asp102, Val190, Cys191, Phe192, Asp194, Ser195, Ala213, Ser214, Phe215, Val216, Cys220, and Ala227, the atomic coordinates being listed in Tables 1 and 2, sequence numbering according to Table 3 as defined by Navia (Navia M A et al., 1989, Proc. Natl. Acad. Sci. USA 86: 7-11). The binding site may display flexibility upon inhibitor binding wherein the loop element Tyr-94-Asp95-Pro98-Val99-Asn99A-Leu99B-Leu100-Asn101 may shift its position in response to inhibitor binding. Wherein such movement is characterised by the position of all atoms of residue Leu99B in relation to the main chain atoms of residues Val190, Cys191, Phe192, Asp194, Ser195, Ala213, Ser214, Phe215, and Val216 and wherein the atomic ensemble comprising all atoms of residue Leu99B and the main chain atoms of residues Val190, Cys191, Phe192, Asp194, Ser195, Ala213, Ser214, Phe215, and Val216 of any given crystalline form of human neutrophil elastase has a root-mean square deviation of more than 0.5 Å from the position of the corresponding atomic ensemble comprising all atoms of residue Leu99B and the main chain atoms of residues Val190, Cys191, Phe192, Asp194, Ser195, Ala213, Ser214, Phe215, and Val216 of any given different crystalline form. In another embodiment and more specifically, the adopted position of the loop element Tyr94-Asp95-Pro98-Val99-Asn99A-Leu99B-Leu100-Asn101 in any given crystalline form is characterised by its main chain phi and psi angles wherein the first crystal form has main chain phi, psi angles of (−61±4°, 121±4°) for Tyr94, (−108±4°, 104±4°) for Asp95, (−74±4°, −11±4°) for Pro98, (−91±4°, 47±4°) for Val99, (−98±4°, 0±4°) for Asn99A, (44±4°, 61±4°) for Leu99B, (−102±4°, 147±4°) for Leu100, and (53±4°, 50±4°) for Asn101 and wherein the second crystal form has main chain phi, psi angles of (−35±4°, 118±4°) for Tyr94, (−124±4°, 90±4°) for Asp95, (−61±4°, −30±4°) for Pro98, (−75±4°, −34±4°) for Val99, (−92±4°, −1±4°) for Asn99A, (58±4°, 43±4°) for Leu99B, (−115±4°, 153±4°) for Leu100, and (60±4°, 46±4°) for Asn101.

In another embodiment, the second crystalline form additionally comprises human neutrophil elastase inhibitors in complex with human neutrophil elastase including any of the above given embodiments of the second crystalline form.

Another aspect of the invention relates to a method of designing a human neutrophil elastase modulator using the atomic coordinates of a crystalline form according to any of the above given embodiments.

Another aspect of the invention relates to a method of selecting a human neutrophil elastase chemical modulator using the atomic coordinates of a crystalline form according to any of the above embodiments.

Another aspect of the invention relates to a method designing or selecting a human neutrophil elastase chemical modulator comprising the steps of: exploring the atomic coordinates of human neutrophil elastase given in Tables 1 and 2 for information on the three-dimensional characteristics of the protein surface; arriving at an alternative overlapping or non-overlapping binding pocket to the active site inhibitor binding pocket; and selecting or designing a human neutrophil elastase modulator using the binding pocket information.

Another aspect of the invention relates to the method of determining the three-dimensional structure of a crystal form of human neutrophil elastase, referred to as a different or new crystal or crystal form of human neutrophil elastase, comprising the step of applying Fourier or molecular replacement methods using the atomic coordinates of a crystal of human neutrophil elastase as given in Table 1 or 2 to model the structure of a new crystal, wherein the active site binding pocket of the new crystal is equivalent to that in the first crystal. In a specific embodiment, the invention is a method of determining the three-dimensional structure of a crystal form of human neutrophil elastase comprising the step of applying difference Fourier or molecular replacement methods using the atomic coordinates of an original (first) crystal of human neutrophile elastase (from Table 1 or 2) to model the structure of a new crystal or new crystal from of human neutrophil elastase, wherein the active site binding pocket of the new crystal is equivalent to that in the original (first) crystal.

In particular provided herein are crystalline forms of a polypeptide including the catalytic domain of human neutrophil elastase. The catalytic domain may be found within the complete protein or within a fragment of the protein and may be also derived from a human neutrophil elastase mutant, homologue or variant. A mutant is a wild type human neutrophil elastase protein having one or more changes in its amino acid sequence. A human neutrophil elastase mutant may have the same activity as the wild type protein, may have modified activity or may be inactive. A variant is a wild type or mutant protein having one or more portions of its sequence removed, or an additional sequence or sequences added, so that the variant is a different length from the wild type or mutant protein. A variant usually has the same activity as the original wild type or mutant protein. A homologue is a related protein in which some parts of the amino acid sequence are similar or the same as in the original protein. Neutrophil elastase from canine, for example, is a homologue of human neutrophil elastase.

According to a further aspect of the invention, we provide a crystalline form of human neutrophil elastase in complex with a small molecular weight inhibitor molecule. As an example, the inhibitor might be a molecule synthesized chemically. Such molecules include, for example, a compound depicted in formula I.

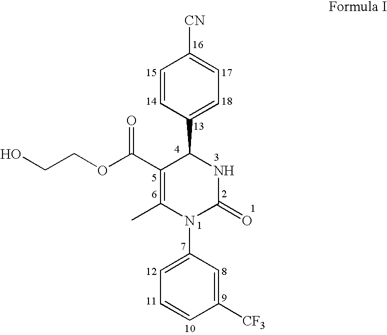

Another aspect of the invention is the unique shape of the S2-subsite in the active site binding pocket in un-complexed human neutrophil elastase and in human neutrophil elastase complexed with an inhibitor of formula I. Using X-ray crystallography, we have determined the three-dimensional molecular structures of an un-complexed human neutrophil elastase and a human neutrophil elastase inhibited with an inhibitor of formula I. Resulting from this, we have determined the unique shape of the S2-subsite in the active site binding pocket of un-complexed human neutrophil elastase and of human neutrophil elastase inhibited with an inhibitor of formula I (defined by the atomic coordinates of its constituent amino acids). Furthermore, we have determined the spatial arrangement of an inhibitor molecule of formula I relative to the human neutrophil elastase active site binding pocket. In particular, we have determined the spatial arrangement of such inhibitor relative to the S2-subsite constituent amino acid residues of so inhibited human neutrophil elastase. This structural information can be stored on a computer-readable medium and may be used for rational drug design.

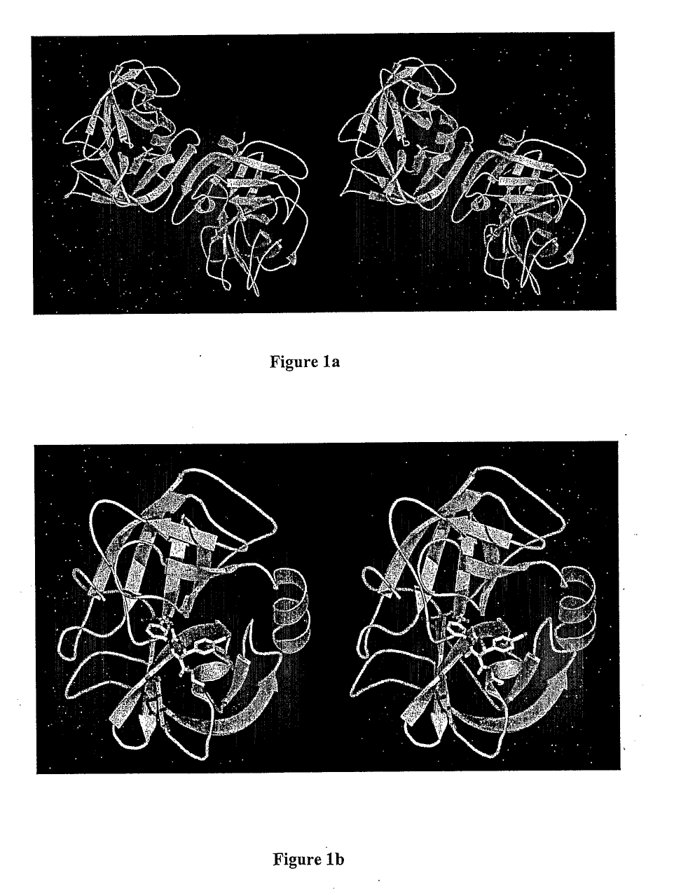

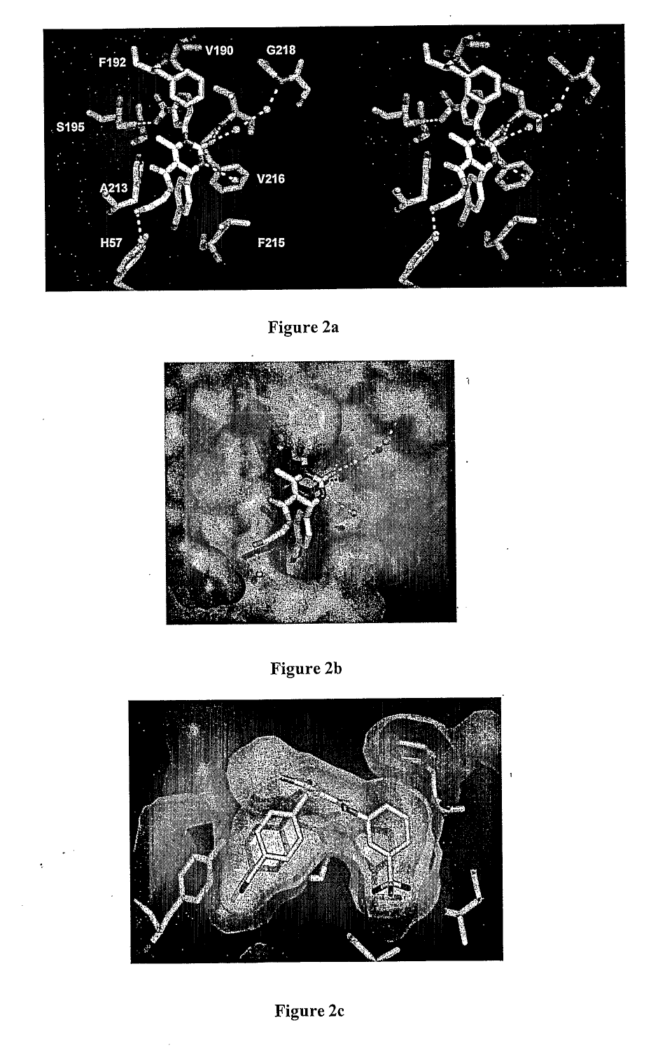

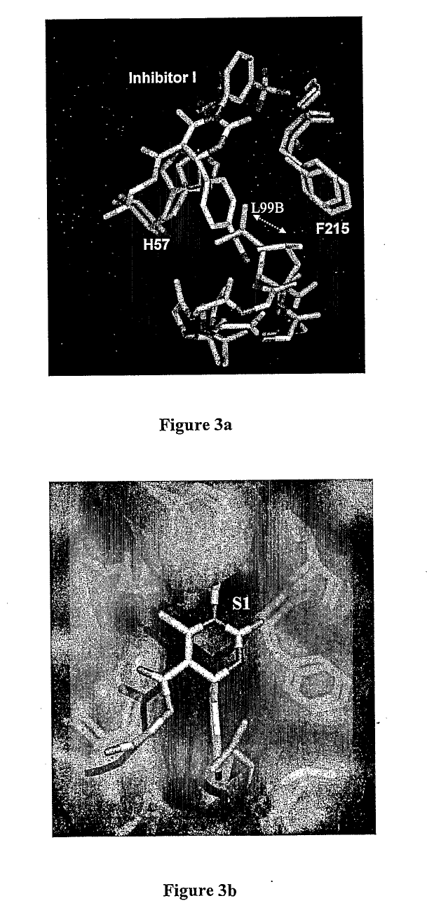

The overall structure of human neutrophil elastase in complex with the inhibitor of formula I is shown in FIG. 1b; more detailed views of the interaction of the inhibitor of formula I with human neutrophile elastase are given in FIGS. 2a and 2b. The overall structure of un-complexed human neutrophil elastase is shown in FIG. 1b; more detailed views of the differences in the active site binding pocket of un-complexed and inhibited human neutrophil elastase are given in FIGS. 3a and 3b.

The shape of the active site binding pocket is defined by the atomic coordinates of the atoms in the amino acid residues in Tables 1 and 2. Table 1 lists the atomic coordinates for the two independent molecules of un-complexed human neutrophil elastase in Protein Data Bank (PDB) format, as determined from the first crystalline form. Table 2 lists the atomic coordinates for human neutrophil elastase together with the inhibitor of formula I, in PDB format, as determined from the second crystalline form. The atomic coordinates are listed in those lines that begin with the code ATOM or HETATM, one atom per line. Following the code are: the unique atom number, the atom name, the amino acid residue mane, the protein chain identifier, the amino acid residue number, the atomic coordinates x, y, and z in orthogonal Angstrom space, the atomic occupancy factor, the atomic temperature factor, and the atom type. The atomic coordinates of the sugar residues linked to human neutrophil elastase carry the residue names of NAG and FUC; the atomic coordinates of the inhibitor I carry the residue name RSU. Solvent water molecules carry the residue name of HOH, and bound sulfate ions derived from the crystallisation buffer carry the residue name of SO4. It is possible to reproduce the shape of the human neutrophil elastase active side binding pocket through carrying out similar structure determinations with minor variations in the experimental conditions (including variations in the protein employed such as mutants, variants and homologues, variations in crystal conditions, crystal form, trial model used in molecular replacement, etc.). Different experiments may give rise to apparently different coordinates, but those skilled in the art will realise that two apparently different sets of coordinates for the same or similar proteins can be shown to be equivalent by superposition of the molecules. For example, the coordinates in Tables 1 and 2 are different numerically. But following superposition they can be seen to describe the same molecule. It will be appreciated that, according to accepted practice, the atomic coordinates may vary within certain limits due to experimental variation. Such variation includes standard experimental error (coordinates determined for the same protein may vary somewhat, for example within 0.3 Å) and other variation (for example, coordinates of human neutrophil elastase mutants, variants, or homologues). The coordinates of the active site binding pocket of human neutrophil elastase may also differ upon introduction of a different small molecule inhibitor, where flexible portions of the binding site adopt a new conformation specific to a type of inhibitor. For example, while the protein coordinates in Table 1, following superposition, in general are seen to be marginally different to those in Table 2, this is not true for particular areas of the active site binding pocket of human neutrophil elastase, where these regions show significantly different coordinates upon inhibitor binding.

The inhibitor molecule (formula I) occupies the central active site binding pocket cleft addressing the S1 subsite with its m-trifluoromethyl-phenyl moiety and the S2 subsite with its p-cyano-phenyl moiety. The trifluoromethyl-phenyl moiety inhibitor I extends deeply into the hydrophobic S1 pocket of the protease and interactions are mainly based on van-der-Waals contacts formed between the aromatic inhibitor moiety and the hydrophobic residues Val190, Ala213 and Val216 of the enzyme. In addition, the phenyl ring of Phe192 is situated lid-like over the aromatic ring of the inhibitor's trifluoromethyl-phenyl moiety stacking in an edge-to-face-fashion against the aryl-ring of the inhibitor with a centroid separation distance of 4.7 Å. One fluorine of the inhibitor is within hydrogen bonding distance (3.15 Å) to the γ-oxygen of the catalytic residue Ser195. The inhibitor interacts with Val216 via a hydrogen bond between the carbonyl-O of the central pyrimidine ring system and the backbone amide of Val-216.

Within the complex of inhibitor I and human neutrophil elastase the S2 subsite forms a deep, hydrophobic pocket which is occupied by parts of the central pyrimidine ring and the p-cyano-phenyl moiety of the inhibitor. As a particular striking feature, it is important to note that binding to this subsite is governed by almost exact shape complementarity of the inhibitor and the protein. The most significant difference, in the active site topology between the apo-structure and the inhibited complex, establishing the unique shape of the active site binding pocket of human neutrophil elastase inhibited by inhibitor I, is found within the S2 binding pocket. The main chain loop containing the consecutive residues Tyr94, Asp95, Pro98, Val99, Asn99A, Leu99B, Leu100, and Asn101 is situated in proximity to the S2 subsite and its sidechains contribute to the panelling of the S2 subsite. It adopts a beta-sheet like topology with several internal hydrogen bonds. The arrangement of this loop can be characterized by the main chain phi, psi angles of the involved amino acid residues as listed above; when the spatial arrangements of this loop within the apo-structure and the inhibited structure are compared, significant differences can be observed, which in another embodiment, are also established by a maximal main chain displacement of about 2.1 Å. From the three-dimensional structure of the complex of human neutrophil elastase with inhibitor I that we have determined, we established this unique shape of the active site binding pocket, which in turn is leading to the effective inhibition of human neutrophil elastase by inhibitor L and is uniquely defined by the atomic coordinates of the constituent amino acid residues of the active site binding pocket, the coordinates being listed in Table 2.

Thus, according to a further aspect of the invention, we provide the unique shape of the active site binding pocket of human neutrophil elastase in response and as a result of binding of inhibitors of formula II to the active site binding pocket of human neutrophil elastase as defined by the atomic coordinates given in table 2 or by equivalent coordinates.

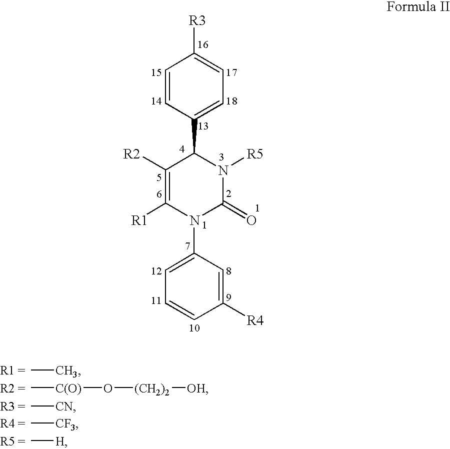

This unique shape of the active site binding pocket of human neutrophil elastase, developed in response to binding of inhibitors of formula II, where the inhibitor of formula I is a specific example, is characterised by the following criteria, including but not limited to:
(i) the m-R4-phenyl moiety of formula II placed in the S1 subsite of the active site binding pocket of human neutrophil elastase, inserting its m-R4 moiety and its aromatic ring system between the lining side chains of Val190, Phe 192, Asp194, Ser195, Ala213, Val 216 and the lining main chain elements of Cys191-Phe192 and of Phe215;
(ii) the central pyrimidinone ring of formula II lying above the side chain of His57 and the main chain element Ser214-Val216;
(iii) the p-R3-phenyl moiety of formula II placed in the S2 subsite of the active site binding pocket of human neutrophil elastase, inserting its p-R3 moiety and its aromatic ring system between the lining side chains of His57, Tyr94, Pro98, Leu99B, Asp102, Ser214, and Phe215;
(iv) a relative orientation of the p-R3-phenyl moiety to the central pyrimidinone ring as defined by the N3-C4-C13-C14 torsion angle of 120±10°;
(v) a relative orientation of the m-R4-phenyl moiety to the central pyrimidinone ring as defined by the C2-N1-C7-C12 torsion angle of −80±10°;
(vi) m-R4-phenyl ring centroid distances of 5.45±0.15 Å to Ser195 Cβ, 5.37±0.15 Å to Phe192 Cβ, 4.69±0.15 Å to Val 215 Cβ, and 6.93±0.15 Å to Ala213 Cβ;
(vii) pyrimidinone ring centroid distances of 4.63±0.15 Å to Phe215 Cβ, 6.02±0.15 Å to His57 Cβ, and 7.67±0.15 Å to Phe192 Cβ;
(viii) p-R3-phenyl ring centroid distances of 4.08±0.15 Å to His57 Cβ, 4.50±0.15 Å to Phe215 Cβ, 5.63±0.15 Å to Leu99B Cβ, 5.73±0.15 Å to Pro98 Cα, 9.39±0.15 Å to Tyr94 Cβ, and 6.41±0.15 Å to Asp102 Cβ;
(ix) m-R4-phenyl ring atom distances of 4.37±0.15 Å for C11-Val216 Cβ, 4.79±0.15 Å for C12-Val216 Cβ, 4.70±0.15 Å for C9-Ser195 Cβ, and 4.74±0.14 Å for C8-Ser195 Cβ;
(x) pyrimidinone ring atom distances of 3.79±0.15 Å for C2-Phe215 Cβ, 4.10±0.15 Å for N3-Phe 215 Cβ, 6.70±0.15 Å for C5-Phe192 Cβ, and 7.80±0.15 Å for C6-Phe192 Cβ;
(xi) a pyrimidinone O1-Val216 main chain N distance of 3.23±0.15 Å; and
(xii) p-R3-phenyl ring atom distances of 5.14±0.15 Å for C18-Leu99B Cγ, 4.45±0.15 Å for C17-Leu99B Cγ, 4.19±0.15 Å for C14-His57 Cβ, and 3.91±0.15 Å for His57 Cβ.

Knowledge of the three-dimensional structure of un-complexed human neutrophil elastase and of human neutrophil elastase inhibited by inhibitors of formula II provides a means for investigating the mechanism of action of the protein and tools for identifying inhibitors of its function. Knowledge of the three-dimensional structure of un-complexed human neutrophil elastase and of human neutrophil elastase inhibited by inhibitors of formula II allows one to design molecules capable of binding to human neutrophil elastase, and more preferably to design molecules capable of binding to human neutrophil elastase and mimicking the binding mode of inhibitors of formula I, and even more preferably to design molecules capable of binding to human neutrophil elastase and mimicking the binding mode of inhibitors of formula II, including molecules which are capable of inhibiting (partially or completely) the activity of human neutrophil elastase.

Illustrative crystalline forms of polypeptides of this invention having various physicochemical properties are disclosed herein. Preferred crystalline form inventions are capable of diffracting X-rays to a resolution better than about 3.0 Å, and more preferably to a resolution of 2.5 Å or better, and even more preferably to a resolution of 2.0 Å or better, and are useful for determining the three-dimensional structure of the material.

Structural coordinates of a crystalline composition of this invention may be stored in a machine-readable form on a machine-readable storage medium, such as a computer hard drive, diskette, DAT tape, CD-ROM, DVD, for display as a three-dimensional shape or for other uses involving computer-assisted manipulation of, or computation based on, the structural coordinates or the three-dimensional structures they define. For example, data defining the three-dimensional structure of the protein of human neutrophil elastase, or portions or structurally similar homologues of such a protein, may be stored in a machine-readable storage medium and displayed as a three-dimensional representation of the protein structure, typically using a computer capable of reading the data from said storage medium and programmed with instructions for creating the representation from such data. The invention thus encompasses a machine, such as a computer, having a memory which contains data representing the structural coordinates of a crystalline composition of this invention, such as the coordinates set forth in Tables 1 and 2, together with additional optional data and instructions for manipulating such data. Such data may be used for a variety of processes, such as the elucidation of other related structures and drug discovery. For example, a first set of such machine-readable data may be combined with a second set of machine-readable data using a machine programmed with instructions for using the first data set and the second data set to determine at least a portion of the coordinates corresponding to the second set of machine-readable data. For instance, the first set of data may comprise a Fourier transform of at least a portion of the coordinates of human neutrophil elastase set forth in Tables 1 and 2, while the second data set may comprise X-ray diffraction data of a molecule or a molecular complex.

More specifically, one of the objects of this invention is to provide three-dimensional structural information on new complexes of human neutrophil elastase. The structural coordinates of a crystalline composition of this invention, or portions thereof, can be used to solve, e.g. by molecular replacement, the three-dimensional structure of a crystalline form of such a polypeptide or polypeptide complex. Doing so involves obtaining X-ray diffraction data for crystals of the polypeptide or polypeptide complex (e.g. in complex with an inhibitor such as a synthetic inhibitor) for which one wishes to determine the three-dimensional structure. The three-dimensional structure of that polypeptide or complex is determined by analyzing the X-ray diffraction data using molecular replacement techniques with reference to the structural coordinates provided. For example, molecular replacement can use a molecule having a known structure as a starting point to model the structure of an unknown crystalline sample. This technique is based on the principle that two molecules which have similar structures, orientations and positions in the unit cell diffract similarly. The term “molecular replacement” refers to a method that involves generating a preliminary model of a crystal whose atomic coordinates are not known, by orienting and positioning a related molecule whose atomic coordinates are known. Phases are then calculated from this molecule, oriented and positioned in the unit cell of the crystal of unknown structure and combined with observed amplitudes to give an approximate Fourier synthesis of the structure whose coordinates are unknown. Molecular replacement involves positioning the known structure in the unit cell in the same location and orientation as the unknown structure. This involves rotating the known structure in the six dimensions (three angular and three spatial dimensions) until alignment of the known structure with the experimental data is achieved. This approximate structure can be refined to yield a more accurate and often higher resolution structure using various refinement techniques. For example, the resultant model for the structure defined by the experimental data may be subjected to rigid body refinement in which the model is subjected to limited additional rotation and translation in the six dimensions yielding positioning shifts of under about 5%. The refined model may then be further refined using other known refinement methods. For example, one may use molecular replacement to exploit a set of coordinates such as set forth in Table 1 or Table 2 to determine the structure of human neutrophil elastase in complex with other compounds than the inhibitors of formula I or II.

The present invention also relates to a method of producing modulators of human neutrophil elastase, preferably inhibitors, which modulate, preferably inhibit, the enzymatic activity of human neutrophil elastase. The method comprises computational evaluation of the structures defined by the machine-readable data (as given in the coordinates set forth in Table 1 and 2) for their ability to associate with various chemical entities. The term “chemical entity”, as used herein, refers to chemical compounds, complexes of at least two chemical compounds, and fragments of such compounds or complexes. For instance, a first set of machine-readable data defining the three-dimensional structure of human neutrophil elastase, or a portion or complex thereof, is combined with a second set of machine-readable data defining the structure of a chemical entity or moiety of interest using a machine programmed with instructions for evaluating the ability of the chemical entity or moiety to associate with the human neutrophil elastase protein or portion or complex thereof and/or the location and/or orientation of such association. Such methods provide insight into the location, orientation and nergetics of association of human neutrophil elastase with such chemical entities. Chemical entities that associate or interact with human neutrophil elastase may inhibit its interaction with naturally occurring ligands for the protein and may inhibit biological functions mediated by such interaction. Such chemical entities are drug candidates.

The protein structure encoded by the data may be displayed in a graphical format permitting visual inspection of the structure, as well as visual inspection of the structure's association with chemical entities. Alternatively, more quantitative or computational methods may be used. For example, one method of this invention for evaluating the ability of a chemical entity to associate with any of the molecules or molecule complexes set forth herein comprises the steps of: (i) employing computational means to perform a fitting operation between the chemical entity and a binding pocket or other surface feature of the molecule or molecular complex; and (ii) analyzing the results of the fitting operation to quantify the association between the chemical entity and the binding pocket.

This invention further provides for the use of the structural coordinates of a crystalline composition of this invention, or portions thereof, to generate and visualize a molecular surface, such as a water-accesible surface or a surface comprising the space-filled van der Waals surface of all atoms; to calculate and visualize the size and shape of surface features of the protein or complex, e.g. ligand binding pockets; to locate potential H-bond donors and acceptors within the three-dimensional structure, preferably within or adjacent to a ligand binding site; to calculate regions of hydrophobicity and hydrophilicity within the three-dimensional structure, preferably within or adjacent to the protein surface of favourable interaction energies with respect to selected functional groups of interest (e.g. amino, hydroxyl, carbonyl, methylene, alkyl, alkenyl, aromatic carbon, aromatic rings, heteroaromatic rings, etc.). One may use the foregoing approaches for characterizing the protein and its interactions with moieties of potential ligands to design or select compounds capable of specific binding to the active site binding pocket and to design or select compounds of complementary characteristics (e.g. size, shape, charge, hydrophobicity/hydrophilicity, ability to participate in hydrogen bonding, etc.) to surface features of the protein, a set of which may be pre-selected. Using the structural coordinates, one may also predict or calculate the orientation, binding constant or relative affinity of a given ligand to the protein in the complexed state, and use that information to design or select compounds of improved affinity.

In such cases, the structural coordinates of human neutrophil elastase as set forth in Tables 1 and 2, or a portion or complex thereof, are entered in machine-readable form into a machine programmed with instructions carrying out the desired operation and containing any necessary additional data (e.g. data defining structural and/or functional characteristics of a potential ligand or moiety thereof and data defining the molecular characteristics of the various amino acids). One method of this invention provides for selecting from a database of chemical structures a molecular compound capable of binding to human neutrophil elastase (e.g. coordinates defining the three-dimensional structure of human neutrophil elastase or a portion thereof). Points associated with the three-dimensional structure (structural coordinates) of a crystalline form of human neutrophil elastase are characterized with respect to the favourability of interactions with one or more functional groups. A database of chemical structures is then searched for candidate compounds containing one or more functional groups disposed for favourable interaction with the protein based on the prior characterization. Compounds having structures which best fit the points of favourable interaction with the three-dimensional structure are thus identified. It is often preferred, although not required, that such searching be conducted with the aid of a computer. In that case a first set of machine-readable data defining the three-dimensional structure of human neutrophil elastase, or a portion or complex thereof, is combined with a second set of machine-readable data defining one or more moieties or functional groups of interest, using a machine programmed with instructions for identifying preferred locations for favourable interaction between the functional group(s) and atoms of the protein. A third set of data, which defines the location(s) of favourable interaction between protein and functional group(s) is generated. The third set of data is then combined with a fourth set of data defining the three-dimensional structures of one or more chemical entities using a machine programmed with instructions for identifying chem. Ical entities containing functional groups to best fit the locations of their respective favourable interaction with the protein. Compounds of the structures selected or designed by any of the foregoing means may be tested for their ability to bind to human neutrophil elastase, inhibit the binding of human neutrophil elastase to a natural or non-natural ligand, and/or inhibit a biological function mediated by human neutrophil elastase.

According to a further aspect of the invention, we provide a method to select or design chemical modulators (Preferably inhibitors) of human neutrophil elastase by using the un-complexed human neutrophil elastase structure (including that of homologues, variants and mutants) and the shape of the active site binding pocket of human neutrophil elastase (including that of homologues, variants and mutants) in complex with inhibitor I. Information from the three-dimensional atomic coordinates of the inhibitor I molecule, its spatial orientation in relation to the three-dimensional atomic coordinates of human neutrophil elastase, and the shape of the active site binding pocket in inhibitor I complexed human neutrophil elastase as given by the three-dimensional atomic coordinates of the active site binding pocket in inhibitor I complexed human neutrophil elastase is used as a tool to design modulators (preferably inhibitors) of human neutrophil elastase. Small-molecule modulators (preferably inhibitors) of human neutrophil elastase may be selected or designed to fit into the shape of the active site binding pocket and/or cause conformational changes in the active site binding pocket of human neutrophil elastase similar to those observed upon binding of inhibitor I.

As described above, the human neutrophil elastase crystal structures as given by the three-dimensional atomic coordinates as set forth in Table 1 and 2 may be used in the rational design of drugs which modulate (preferably inhibit) the action of human neutrophil elastase. These human neutrophil elastase modulators may be used to prevent or treat undesirable physical, physiological and pharmacological consequences of inappropriate activity of human neutrophil elastase.

The present invention will now be described with reference to the following non-limiting examples.

### EXAMPLES

### Example 1

### Crystallization of Apo Human Neutrophil Elastase

Human neutrophil elastase purified from human placenta was obtained from Serva, Heidelberg, and crystallized by vapour diffusion using the sitting drop method. The lyophilized enzyme was dissolved in 10 mM Hepes/NaOH, pH 6.5 to a final concentration of 10 mg/ml. Crystals were grown at 20° C. in 1.9 M ammonium sulfate, 5% (w/v) PEG 400, 0.1 M MES/NaOH, pH 6.5, 20% (v/v) glycerol as reservoir solution. Droplets were prepared by diluting 1 μl protein solution in 1 μl reservoir solution to yield a final 2 μl drop volume. For diffraction experiments crystals were directly flash-frozen in liquid nitrogen. The crystals are tetragonal and belong to space group P4₁2₁2, with unit cell parameters a=b=123.22 Å, c=68.82 Å, α=β=γ=90°. The asymmetric unit contains two molecules of human neutrophil elastase.

### Example 2

### Synthesis of Inhibitor I

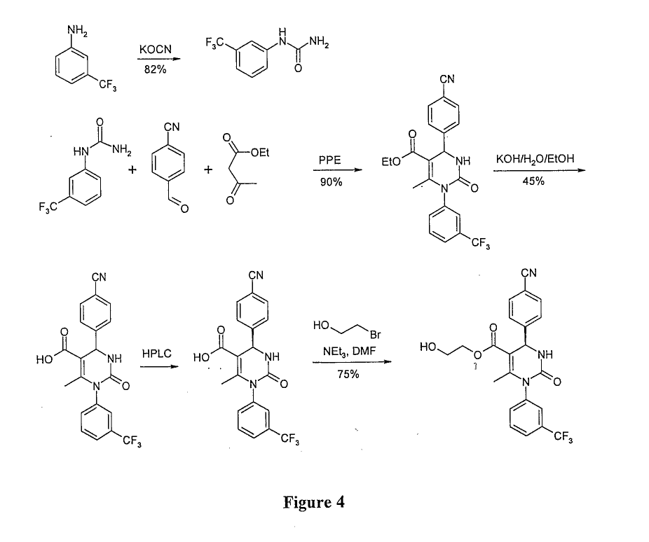

Preparation of inhibitor I is described under example 72 in patent application WO 2004/024700, publication date 25, Mar. 2004. Briefly, inhibitor I is synthesized from 3-trifluoromethylaniline, 4-cyanobenzaldehyde, ethyl-3-oxobutanoate, and 2-bromoethanol in 5 steps as depicted in FIG. 4.

### Ethyl 4-(4-cyanophenyl)-6-methyl-2-oxo-1-[3-(trifluoromethyl)phenyl]-1,2,3,4-tetrahydro5-pyrimidinecarboxylate

7.0 g (34.29 mmol) N-[3-(trifluoromethyl)phenyl]urea, 8.99 g (68.58 mmol) 4-cyanobenzaldehyde, 8.92 g (68.58 mmol) ethyl 3-oxobutanoate and 20 g polyphosphoric acid ethyl ester were suspended in 250 ml of tetrahydrofuran. The mixture was stirred at reflux for 18 hours. After cooling down to room temperature, the solvent was removed in vacuo and the residue was purified by column chromatography on silica with cyclohexane/ethyl acetate as eluent. Yield: 13.4 g (91% of th.)

¹H-NMR (200 MHz, DMSO-d₆): δ=1.1 (t, 3H); 2.0 (s, 3H); 4.0 (q, 2H); 5.4 (d, 1H); 7.6 (m, 3H); 7.7 (m, 3H); 7.9 (m, 2H); 8.4 (d, 1H) ppm.

### 4-(4-Cyanophenyl)-6-methyl-2-oxo-1-[3-(trifluoromethyl)phenyl]-1,2,3,4-tetrahydro-5-pyrimidinecarboxylic acid

3 g (7 mmol) of ethyl 4-(4-cyanophenyl)-6-methyl-2-oxo-1-[3-(trifluoromethyl)phenyl]-1,2,3,4-tetra-hydro-5-pyrimidinecarboxylate were dissolved in a mixture of 50 ml water and 100 ml 5% KOH in ethanol. The reaction mixture was stirred at room temperature for 18 hours. The solvent was removed in vacuo and the residue was purified by column chromatography on silica with dichloromethane/methanol as eluent. Yield: 1.27 g (45% of th.)

¹H-NMR (300 MHz, DMSO-d₆): δ=2.0 (s, 3H); 5.4 (d, 1H); 7.6 (m, 1H); 7.6 (m, 2H); 7.7 (m, 1H); 7.8 (m, 1H); 7.9 (m, 3H); 8.3 (d, 1H); 12.5 (s, 1H) ppm.

### (4R)-4-(4-Cyanophenyl)-6-methyl-2-oxo-1-[3-(trifluoromethyl)phenyl]-1,2,3,4-tetrahydro-5-pyrimidinecarboxylic acid

The enantiomers of 4-(4-cyanophenyl)-6-methyl-2-oxo-1-[3-(trifluoromethyl)phenyl]-1,2,3,4-tetrahydro-5-pyrimidinecarboxylic acid were separated by preparative HPLC on a chiral phase [column KBD 8361 (chiral silica gel selector based on monomer N-methacryloyl-L-leucine-1-menthylamide, cf. EP-A-379 917), 250 mm×20 mm, eluent: ethyl acetate→methanol→ethyl acetate, flow 25 ml/min, temperature 23° C., detection 254 nm].

[α]²⁰=+2.5°(λ=589 nm, methanol, c=505 mg/100 ml).

### (4R)-2-Hydroxyethyl 4-(4-cyanophenyl)-6-methyl-2-oxo-1-[3-(trifluoromethyl)phenyl]-1,2,3,4-tetrahydro-5-pyrimidinecarboxylate

Under argon, 1560 mg (3.89 mmol) (4R)-4-(4-cyanophenyl)-6-methyl-2-oxo-1-[3-(trifluoromethyl)phenyl]-1,2,3,4-tetrahydro-5-pyrimidinecarboxylic acid were added to 19.6 ml dimethyl formamide. After addition of 1.095 ml (7.86 mmol) triethylamine and 1.11 ml (15.7 mmol) 2-bromoethanol, the reaction mixture was stirred at ca. 70° C. for 8 hours. After cooling, the reaction mixture was concentrated in vacuo. The residue was taken up in ethyl acetate and washed with water. After drying with magnesium sulfate, the organic phase was evaporated in vacuo. The residue was taken up in 8 ml methanol and purified by preparative HPLC (column: Nucleosil 100-5 C 18 Nautilus, 20×50 mm, 5 μm; solvent A: acetonitrile, solvent B: water+0.3% formic acid; gradient: 0 min 10% A, 2 min 10% A, 6 min 90% A, 7 min 90% A, 7.1 min 10% A, 8 min 10% A; wavelength: 220 nm; injection volume: ca. 500111; number of injections: 18). The product containing fractions were combined and lyophilized. Yield: 1.3 g (75% of th.)

MS (EI): m/z=446 (M+H)⁺

¹H-NMR (300 MHz, DMSO-d₆): δ=2.05 (d, 3H); 3.5 (quartett, 2H); 3.95-4.15 (m, 2H); 4.75 (tr, 1H); 5.45 (d, 1H); 7.55-7.75 (m, 5H); 7.75 (d, 1H); 7.85 (d, 2H); 8.35 (d, 1H) ppm.

[α]²⁰=+14.3°(λ=589 nm, methanol, c=455 mg/100 ml).

### Example 3

### Crystallization of Human Neutrophil Elastase in Complex with Inhibitor I

Human neutrophil elastase purified from human placenta was obtained from Serva, Heidelberg, and crystallized by vapour diffusion using the sitting drop method. The lyophilized enzyme was dissolved in 10 mM Hepes/NaOH, pH 6.5 to a final concentration of 10 mg/ml. For crystallization, a 250 mM stock solution of inhibitor I in DMSO was added to the enzyme to give a final concentration of 1 mM in 7% DMSO, the molar ratio of inhibitor to enzyme was 3:1. Crystals were grown at 20° C. using 20% (w/v) PEG 3350, 0.2 M (NH₄)₂citrate, pH 5.0 as reservoir solution. Droplets were prepared by diluting 0.8 μl protein solution with an equal volume of the reservoir solution. Crystals were harvested in reservoir solution, soaked for 10 seconds in cryoprotection solution containing 20% (w/v) PEG 3350, 0.2 M (NH₄)₂citrate, pH 5.0 with 20% (v/v) glycerol and flash-frozen in liquid nitrogen. The crystals are hexagonal and belong to the space group P6₃ with lattice constants a=b=73.73 Å, c=70.66 Å, α=β=90°, γ=120°. The asymmetric unit contains one molecule of human neutrophil elastase.

### Example 4

### Data Collection, Structure Determination and Refinement of Apo Human Neutrophil Elastase

X-ray diffraction data were collected by the rotation method using synchrotron radiation of beamline X31 (EMBL Outstation at DESY, Hamburg) at a wavelength of 1.1 Å and a MAR image plate detector (MAR45, Mar Research, Hamburg). The data of the HNE apo-enzyme was processed using MOSFLM and SCALA included in the CCP4 program package (Collaborative Computational Project Number 4, 1994, Acta Cryst. D50: 760-763). A total of 1599060 observations in the resolution range 41-1.86 Å were reduced to 42465 unique reflections. Crystal mosaicity was refined to 0.31°. The merged data set contained 99.4% of the reflections expected in this resolution range, the overall R_{merge} was 12.0% and 38.6% for the highest resolution range (1.96-1.86 Å). The structure was solved by molecular replacement using the program AMORE from the CCP4 package with starting coordinates of Human Neutrophil Elastase from the HNE:peptide chloromethyl ketone inhibitor complex^{[20]}, as deposited in the entry 1HNE (Navia M A et al., Proc. Natl. Acad. Sci. U.S.A., 86: 7-11) in the Protein Data Bank (Berman H M et al., 2000, Nuc. Acids Res., 28: 235-242). The structure was refined using the program REFMAC from the CCP4 package using default parameters alternating with manual model building using the program O (Jones T A et al., 1991, Acta Cryst., A47: 110-119). During the course of refinement, the correctly rotated and translated search model (starting R-factor 0.387) was subjected to rigid-body refinement and subsequently refined to convergence by conventional restrained refinement (R-factor R_{work} 0.242, R_{free} 0.288). The model was manually adjusted on the basis of 2|Fₒ|-|F_{c}| and |Fₒ|-|F_{c}| electron density maps. Subsequently, water molecules and sugar residues were added. The apo-enzyme was crystallized using ammonium sulfate as precipitant, therefore several sulfate ions were visible in the electron density of the structure. Additional incorporation of sulfate followed by further refinement led to a final structure of the apo-enzyme with an R-factor of 0.172 (free R-factor 0.216). The final model of the apo-enzyme contains 3917 non-hydrogen atoms with 481 water molecules included. RMS deviations were 0.016 Å and 1.8° for bond distances and angles, respectively. Data Collection and final refinement statistics are summarized in table 4.

### Example 5

### Data Collection, Structure Determination and Refinement of Human Neutrophil Elastase Inhibited with Inhibitor I

X-ray diffraction data were collected by the rotation method using synchrotron radiation of beamline X31 (EMBL Outstation at DESY, Hamburg) at a wavelength of 1.1 Å and a MAR image plate detector (MAR345, Mar Research, Hamburg). The data was evaluated by the DENZO/SCALEPACK program package (Otwinowski Z & Minor W, 1997, Meth, Enzymol., 276: 307-326). A total of 396270 observations in the resolution range of 35-2.0 Å were reduced to 14498 unique reflections. Crystal mosaicity was refined to 0.74°. The merged data set contained 97.5% of all reflections expected in this resolution range. The merging R-factor for point-group symmetry related reflections was 0.092 over the complete resolution range and 0.348 for the highest resolution range (2.07-2.0 Å). The structure was solved by molecular replacement using the program from the CCP4 package with starting coordinates of Human Neutrophil Elastase from the HNE:peptide chloromethyl ketone inhibitor complex, as deposited in the entry 1HNE in the Protein Data Bank. Structures were refined using the program REFMAC from the CCP4 package using default parameters alternating with manual model building using the program O. During the course of refinement, the correctly rotated and translated search model (starting R-factor 0.394) was subjected to rigid-body refinement and subsequently refined to convergence by conventional restrained refinement (R-factor R_{work} 0.317, R_{free} 0.251). The model was manually adjusted on the basis of 2|Fₒ|-|F_{c}| and |Fₒ|-|F_{c}| electron density maps. Subsequently, water molecules, sugar residues and the inhibitor molecule were added. Additional cycles of refinement and model adjustment led to a final structure with an R-factor of 0.159 (free R-factor 0.224). The final model consists of 1916 non-hydrogen atoms and includes 197 water molecules. All non-hydrogen atoms were refined with restrained B-factors. The RMS deviations from ideal values for bond distances and angles were 0.017 Å and 1.9°, respectively. Data Collection and final refinement statistics are summarized in Table 5.

### Example 6

### Description of the Structures of Apo Human Neutrophil Elastase and Human Neutrophil Elastase in Complex with Inhibitor I

The overall structures of human neutrophil elastase as observed in its un-complexed form and in its Inhibitor I inhibited form are very similar to the previously published complexes of human neutrophil elastase with peptide and peptidomimetic inhibitors (MacDonald S J et al., 2002, J. Med. Chem., 45: 3878-3890; Cregge R J et al., 1998, J. Med. Chem., 41: 2461-2480; Navia M A et al., 1989, Proc. Natl. Acad. Sci. U.S.A., 86: 7-11; Wei A Z et al., 1988, FEBS Lett., 234: 367-373; Bode W et al., 1986, EMBO J. 5: 2453-2458) with the exception of the area of the active site binding pocket where unique, significantly different and unexpected orientations of the loop element Tyr-94-Asp95-Pro98-Val99-Asn99A-Leu99B-Leu 100-Asn 101 are observed. This specified loop element is a major forming element of the S2 subsite of the active site binding pocket and thereby its topological orientation is the major determinant of the shape of the S2-subsite of the active binding site of human neutrophil elastase. In turn, its position adopted defines the addressable space and shape of the S2 subsite and thereby is a key element for the effectiveness of inhibitors. Specific compounds like Inhibitor I can modulate the position of the specified loop element to shape complementarity towards the employed inhibitor and thereby achieve a unique and unexpected mode of Inhibition. Interestingly, the previously published structures do not show either of the positions adopted in the apo structure or in the inhibitor I inhibited form. Moreover, and of even importance, the degree of shape complementarity observed with inhibitor I is not achieved with native peptide and synthetic peptidomimetic inhibitors. The three-dimensional structures disclosed herein clearly reveal that it is an important and non-obvious characteristic of inhibitor I to modulate the conformation of the specified loop element to fit to the shape of inhibitor I with almost perfect shape complementarity. The unique orientations of the specified loop element are characterised by the position of all atoms of residue Leu99B in relation to the main chain atoms of residues Val190, Cys191, Phe192, Asp194, Ser195, Ala213, Ser214, Phe215, and Val216 and wherein the atomic ensemble comprising all atoms of residue Leu99B and the main chain atoms of residues Val190, Cys191, Phe192, Asp194, Ser195, Ala213, Ser214, Phe215, and Val216 exhibits a root-mean square deviation of more than 0.5 Å from the position of the corresponding atomic ensemble comprising all atoms of residue Leu99B and the main chain atoms of residues Val190, Cys191, Phe192, Asp194, Ser195, Ala213, Ser214, Phe215, and Val216 of the previously published structures. For example, the root-mean square deviations observed for the specified atomic ensemble are 0.616 Å between molecule A of the apo structure of human neutrophil elastase and the structure described by 1HNE (Navia M A et al., 1989, Proc. Natl. Acad. Sci. U.S.A. 86: 7-11), 0.869 Å between the inhibitor I inhibited form of human neutrophil elastase and 1HNE, and even 0.964 Å between the apo structure and the inhibitor I inhibited form of human neutrophil elastase, respectively. In contrast, illustrating the degree of conformational change, the observed root-mean square deviation for the specified atomic ensemble between 1HNE and 1H1B (MacDonald S J et al., 2002, J. Med. Chem., 45: 3878-3890) is as small as 0.178 Å. The adopted position of the loop element Tyr94-Asp95-Pro98-Val99-Asn99A-Leu99B-Leu100-Asn101 may also be described by its main chain phi and psi angles wherein the apo form has main chain phi, psi angles of (−61±4°, 121±4°) for Tyr94, (−108±4°, 104±4°) for Asp95, (−74±4°, −11±4°) for Pro98, (−91±4°, −47±4°) for Val99, (−98±4°, 0±4°) for Asn99A, (44±4°, 61±4°) for Leu99B, (−102±4°, 147±4°) for Leu100, and (53±4°, 50±4°) for Asn101 and wherein the inhibitor I inhibited form has main chain phi, psi angles of (−35±4°, 118±4°) for Tyr94, (−124±4°, 90±4°) for Asp95, (−61±4°, −30±4°) for Pro98, (−75±4°, −34±4°) for Val99, (−92±4°, −1±4°) for Asn99A, (58±4°, 43±4°) for Leu99B, (−115±4°, 153±4°) for Leu100, and (60±4°, 46±4°) for Asn101.

The m-trifluormethyl-phenyl moiety of inhibitor I occupies the S1 subsite while parts of the central pyrimidine ring and the p-cyano-phenyl moiety are positioned in the S2 subsite of the enzyme. The m-trifluoromethyl-phenyl moiety of inhibitor I extends deeply into the hydrophobic S1 pocket of the protease and major interactions are van der Waals contacts formed between the aromatic inhibitor moiety and residues Val 190, Phe192, Asp 194, Ser195, Ala213 Phe215, and Val216 of the enzyme. The phenyl ring of Phe 192 is situated lid-like over the aromatic ring of the inhibitor's m-trifluoromethyl-phenyl moiety stacking in an edge-to-face-fashion against the aryl-ring of the inhibitor with a centroid separation distance of about 4.7 Å. One fluorine of the inhibitor is within hydrogen bonding distance (3.15 Å) to the γ-oxygen of the catalytic residue Ser-195. Inhibitor I also interacts with Val216 via a hydrogen bond between the carbonyl-O of the central pyrimidine ring system and the backbone amide of Val216. The S2 subsite forms a deep, hydrophobic pocket which is occupied by parts of the central pyrimidine ring and the p-cyano-phenyl moiety of the inhibitor. As already mentioned and particular striking, it is important to note that binding to this subsite is governed by exact shape complementarity of inhibitor I and the protein subsite. As also previously discussed, the most significant difference in the active site topology between the apo-structure and the inhibitor I complex is found within the S2 binding pocket. The main chain loop containing the consecutive residues Tyr94, Asp95, Pro98, Val99, Asn99A, Leu99B, Leu 100, and Asn101 is situated in proximity to the S2 subsite and its sidechains contribute to the panelling of the S2 subsite. It adopts a beta-sheet like topology with several internal hydrogen bonds and comparison of its arrangement within the apo and the inhibitor I complex structure reveals a maximum main chain displacement of about 2.1 Å. The side chain of Leu-99B is displaced in the inhibitor I complex (shift of the Cβ-atom: 2.6 Å) and forms one side of the S2 subsite by lining it. In this conformation, Leu-99B is oriented towards the bulk solvent and thereby opens up the uniquely shaped, deep and mainly hydrophobic S2 subsite observed in the inhibitor I complex. This pocket perfectly accommodates the p-cyano-phenyl moiety of the inhibitor. A superposition of the complex structure and the structure of the apo-enzyme reveals that the conformation of Leu99B adopted in the un-complexed enzyme actually positions the side chain of Leu99B within the enlarged S2 pocket as formed when the enzyme is complexed with inhibitor I. Vice versa, the conformation of the sidechain of Leu99B adopted in the un-complexed enzyme leads to a rather restricted and relatively shallow S2 subsite, which will not be able to accommodate the rather large p-cyano-phenyl residue of the inhibitor, demonstrating that by this non-obvious and unexpected mechanism of induced fit the conformation of Leu99B is rearranged to create an S2 subsite of almost perfect spatial complementarity to the p-cyano-phenyl moiety of the inhibitor. In addition to the changes observed in the S2 subsite, the shape of the S4 pocket of HNE is also influenced by the conformational change of Leu-99B. The S4 subsite of the enzyme forms a mainly hydrophobic surface lined by the amino acid residues Leu99B, Phe215, and Arg217. In inhibitor complexes previously published, compounds with aliphatic or aromatic moieties such as alanyl, phenyl or pyrrolidine rings have been found to bind to this subsite. In contrast, within the inhibitor I complex, the inhibitor-induced conformational changes at the S2 subsite cause a movement of Leu99B towards the S4 subsite of the enzyme which in turn leads to a significant change of the shape of this subsite. While the conformation of the sidechain of Leu99B adopted in the structure of the apo-enzyme and other, previously published HNE complexes creates a more pronounced S4 subsite together with a rather narrow S2 subsite, the conformation of Leu99B in inhibitor I complex structure creates a pronounced S2 subsite with a rather restricted S4 subsite.

A limited number of five different crystal structures of human neutrophil elastase in complex with native peptide and synthetic peptidomimetic inhibitors have been reported in the literature. Bode et al. reported a non covalent complex of human neutrophil elastase with the third domain of the ovomucoid inhibitor, a 56 amino acid protease inhibitor (Bode W et al., 1986, EMB O J., 5: 2453-2458). In addition, four complexes with “suicide” synthetic small molecule inhibitors forming covalent bonds with Ser188 and/or His57 have been described (MacDonald S J et al., 2002, J. Med. Chem., 45: 3878-3890; Cregge R J et al., 1998, J. Med. Chem., 41: 2461-2480; Navia M A et al., 1989, Proc. Natl. Acad. Sci. U.S.A., 86: 7-11; Wei A Z et al., 1988, FEBS Lett., 234: 367-373). The majority of these compounds have peptidic core structures and solely the complex formed with GW-475151, a pyrrolidine trans-lactam inhibitor, reported by MacDonald et al. (2002, J. Med. Chem., 45: 3878-3890) represents the only non-peptidic inhibitor structure reported so far. Neither a complex with a non-covalent, non-peptidic inhibitor nor the apo-enzyme structure has been reported before. All previously published inhibitor complexes of human neutrophil elastase reveal inhibitors bound in a substrate-like binding mode, basically exhibiting similar orientation and interactions as observed for the OMTKY3 complex which is the most substrate like complex reported. Whereas OMTKY3 addresses binding pockets on both sides of the scissile peptide bond (S3′-S5), other inhibitors only address non-primed subsites. All complexes with the “suicide” small molecule inhibitors (MeO-Suc-Ala-Ala-Pro-Ala-CH₂Cl, 1HNE, Navia M A et al., 1989, Proc. Natl. Acad. Sci. U.S.A., 86: 7-11; MeO-Suc-Ala-Ala-Pro-Val-CH₂Cl, 1PPE, Wei A Z et al., 1988, FEBS Lett., 234: 367-373; GW475151, 1H1B, MacDonald S J et al., 2002, J. Med. Chem., 45: 3878-3890; and MDL101146, 1BOF, Cregge R J et al., 1998, J. Med. Chem., 41: 2461-2480) reveal covalent bonds with Ser195 and MeO-Suc-Ala-Ala-Pro-Ala-CH₂Cl forms an additional covalent bond with the catalytic residue His57. While the alignment of OMTKY3 and the other peptidomimetic inhibitors is following the alignment of the backbone of a natural substrate in the active site cleft of HNE and may be characterized as colinear with the extension of the active site cleft of HNE, Inhibitor I binds in a unique, non-obvious and unexpected orientation which is almost perpendicular to the alignment of the “backbone” of the other inhibitors.

Thereby, despite its rather compact nature, Inhibitor I can effectively address the S1 and S2 subsites of human neutrophil elastase with its m-trifluoromethyl-phenyl- and p-cyano-phenyl-moieties, respectively. The S1 subsite of the enzyme forms a rather deep and mainly hydrophobic pocket. No significant conformational changes in residues comprising the S1 subsite are observed leading to an almost identical overall shape of the S1 subsite in all complexes including the inhibitor I complex and the apo enzyme. However and interestingly, the most intimate interaction of an inhibitor moiety with the S1 subsite is observed for inhibitor I, where the m-trifluoromethyl-phenyl moiety exhibits the deepest insertion into the S1 binding pocket and occupies the binding cavity to almost perfect shape complementarity. All other inhibitors, including the most “native-like” OMTKY3 inhibitor, show a significantly smaller depth of penetration into the S1 pocket, which is also triggered by the orientation and alignment of the peptide backbone or peptide-like backbone structure of the inhibitors which make use of main-chain main-chain interactions and by the employment of “native-like” Leu or Val residues or moieties mimicking these amino acids in the P1 position.

As already mentioned, a totally different situation, characterized by an effect unexpected and non-obvious in its extent, is encountered for the S2 subsite; here, the native enzyme forms a rather shallow, mainly hydrophobic binding site suitable to accommodate rather small and predominantly hydrophobic amino acid side chains like Ala, Val, Thr, or Pro. The design of the published inhibitors is again mimicking “native-like” structures at the P2 position and employs residues like Pro (1HNE, 1PPG, 1B0F), pyrrolidine (1H1B) or Thr (1PPF) embedded in peptidic core structures. Accordingly, as already observed for the S1-P1 interactions, the binding of the P2 residues to the S2 subsite is dominated by the backbone orientation and conformation packing the P2 moieties against the sidechains of His57 and Leu99B. In contrast, the binding of the “P2-moiety” inhibitor I is totally unique, as its p-cyano-phenyl-moiety inserts deeply into a large and deep S2 cavity, imprinted to the enzyme by inhibitor I and leading to conformational rearrangement of the Tyr94-Asn101 loop element. Again the subsite is well occupied by the inhibitor's moiety and fills the cavity to almost perfect shape complementarity. The linking element between P1 (p-trifluoromethyl-phenyl) and P2 (p-cyano-phenyl) in inhibitor I, the dihydropyrimidinone entity, thereby adopts a position that almost superimposes with the position of the P2 sidechain in peptide-based inhibitors.

For the loop element Tyr94-Asn101, being a substantial part of the S2 subsite of human neutrophil elastase, some flexibility has been described earlier (MacDonald S J et al., 2002, J. Med. Chem. 45: 3878-3890). It has been speculated for the GW475151 complex structure that the binding of the inhibitor causes a dislocation of the main chain as a consequence of a hydrophobic contact between the side chain of Leu99B and the pyrrolidine group of the inhibitor located at the S4 subside of the protease. In this complex the position of the protein backbone of the flexible loop and the Leu99B side chain closely resemble the situation observed in the apo-structure. This arrangement could be regarded as a “closed conformation” in regard to the S2 binding site. An overlay with the other structures of human neutrophil elastase reveals only small differences in the main chain conformation of the Tyr94-Asn101 loop element as well as in the orientation of the Leu99B side chain among the different structures. The orientation of the Leu99B side chain toward the S2 pocket in these structures leads to a shallow S2 binding site with no significant cavity. However and significantly deviating form the previously described situation, the position of the main chain of the Tyr94-Asn101 loop and of the side chain of Leu99B in the inhibitor I complex is clearly different and unique when compared to previously reported inhibitor structures of human neutrophil elastase. Here, inhibitor I enforces a conformation of the Tyr94-Asn101 loop which leads to the formation of the previously described, deep, and well defined S2 binding pocket, which then encompasses the p-cyano-phenyl “P2 residue” of inhibitor I. Accordingly, we may describe the conformation of the flexible loop observed in the inhibitor I complex as an “open conformation” in regard to the binding of inhibitor residues at the S2 pocket.

Presumably, the structures of apo human neutrophil elastase and of human neutrophil elastase in complex with inhibitor I as disclosed herein reveal that the Tyr94-Asn101 loop is a key element for inhibition of human neutrophil elastase by small molecule inhibitors. In particular, addressing the observed structural flexibility of this loop to an extent as revealed with inhibitor I, is an important feature for the creation of strong and effective inhibitors of human neutrophil elastase activity and can be exploited in the design of new inhibitors for human neutrophil elastase.

TABLE 1
Coordinates for the two molecules in the asymmetric unit of
un-complexed human neutrophil elastase in space group P4₁2₁2

| | | | | | | | | | | | |
|---|---|---|---|---|---|---|---|---|---|---|---|
| ATOM | 1 | N | ILE | A | 16 | 110.372 | 86.729 | 9.794 | 1.00 | 8.41 | N |
| ATOM | 2 | CA | ILE | A | 16 | 111.199 | 85.674 | 9.067 | 1.00 | 8.98 | C |
| ATOM | 3 | CB | ILE | A | 16 | 110.433 | 84.366 | 9.027 | 1.00 | 9.18 | C |
| ATOM | 4 | CG1 | ILE | A | 16 | 110.205 | 83.783 | 10.419 | 1.00 | 10.81 | C |
| ATOM | 5 | CD1 | ILE | A | 16 | 111.451 | 83.301 | 11.100 | 1.00 | 11.18 | C |
| ATOM | 6 | CG2 | ILE | A | 16 | 111.071 | 83.333 | 8.117 | 1.00 | 8.67 | C |
| ATOM | 7 | C | ILE | A | 16 | 111.384 | 86.177 | 7.666 | 1.00 | 10.21 | C |
| ATOM | 8 | O | ILE | A | 16 | 110.379 | 86.484 | 6.971 | 1.00 | 10.39 | O |
| ATOM | 9 | N | VAL | A | 17 | 112.637 | 86.276 | 7.233 | 1.00 | 10.40 | N |
| ATOM | 10 | CA | VAL | A | 17 | 112.986 | 86.733 | 5.874 | 1.00 | 11.00 | C |
| ATOM | 11 | CB | VAL | A | 17 | 114.247 | 87.629 | 5.894 | 1.00 | 10.86 | C |
| ATOM | 12 | CG1 | VAL | A | 17 | 114.620 | 88.070 | 4.499 | 1.00 | 11.01 | C |
| ATOM | 13 | CG2 | VAL | A | 17 | 114.078 | 88.825 | 6.842 | 1.00 | 13.48 | C |
| ATOM | 14 | C | VAL | A | 17 | 113.319 | 85.499 | 5.027 | 1.00 | 11.18 | C |
| ATOM | 15 | O | VAL | A | 17 | 114.241 | 84.724 | 5.372 | 1.00 | 10.88 | O |
| ATOM | 16 | N | GLY | A | 18 | 112.612 | 85.329 | 3.920 | 1.00 | 11.39 | N |
| ATOM | 17 | CA | GLY | A | 18 | 112.921 | 84.287 | 2.941 | 1.00 | 12.55 | C |
| ATOM | 18 | C | GLY | A | 18 | 112.368 | 82.904 | 3.267 | 1.00 | 11.52 | C |
| ATOM | 19 | O | GLY | A | 18 | 112.831 | 81.912 | 2.736 | 1.00 | 11.47 | O |
| ATOM | 20 | N | GLY | A | 19 | 111.389 | 82.861 | 4.157 | 1.00 | 11.62 | N |
| ATOM | 21 | CA | GLY | A | 19 | 110.712 | 81.634 | 4.546 | 1.00 | 11.74 | C |
| ATOM | 22 | C | GLY | A | 19 | 109.424 | 81.491 | 3.750 | 1.00 | 11.49 | C |
| ATOM | 23 | O | GLY | A | 19 | 109.301 | 81.988 | 2.662 | 1.00 | 10.51 | O |
| ATOM | 24 | N | ARG | A | 20 | 108.474 | 80.766 | 4.308 | 1.00 | 11.99 | N |
| ATOM | 25 | CA | ARG | A | 20 | 107.204 | 80.446 | 3.650 | 1.00 | 12.92 | C |
| ATOM | 26 | CB | ARG | A | 20 | 107.267 | 79.079 | 2.938 | 1.00 | 13.18 | C |
| ATOM | 27 | CG | ARG | A | 20 | 107.570 | 77.910 | 3.909 | 1.00 | 14.40 | C |
| ATOM | 28 | CD | ARG | A | 20 | 107.432 | 76.488 | 3.388 | 1.00 | 18.96 | C |
| ATOM | 29 | NE | ARG | A | 20 | 107.890 | 75.555 | 4.420 | 1.00 | 20.49 | N |
| ATOM | 30 | CZ | ARG | A | 20 | 109.171 | 75.239 | 4.666 | 1.00 | 22.30 | C |
| ATOM | 31 | NH1 | ARG | A | 20 | 110.164 | 75.718 | 3.910 | 1.00 | 22.17 | N |
| ATOM | 32 | NH2 | ARG | A | 20 | 109.460 | 74.449 | 5.700 | 1.00 | 22.22 | N |
| ATOM | 33 | C | ARG | A | 20 | 106.134 | 80.394 | 4.726 | 1.00 | 13.06 | C |
| ATOM | 34 | O | ARG | A | 20 | 106.462 | 80.263 | 5.906 | 1.00 | 13.27 | O |
| ATOM | 35 | N | ARG | A | 21 | 104.873 | 80.558 | 4.325 | 1.00 | 13.86 | N |
| ATOM | 36 | CA | ARG | A | 21 | 103.748 | 80.368 | 5.223 | 1.00 | 15.03 | C |
| ATOM | 37 | CB | ARG | A | 21 | 102.409 | 80.542 | 4.489 | 1.00 | 16.15 | C |
| ATOM | 38 | CG | ARG | A | 21 | 102.087 | 81.990 | 4.230 | 1.00 | 19.82 | C |
| ATOM | 39 | CD | ARG | A | 21 | 100.858 | 82.226 | 3.328 | 1.00 | 24.16 | C |
| ATOM | 40 | NE | ARG | A | 21 | 101.163 | 83.400 | 2.549 | 1.00 | 32.55 | N |
| ATOM | 41 | CZ | ARG | A | 21 | 101.917 | 83.399 | 1.449 | 1.00 | 36.29 | C |
| ATOM | 42 | NH1 | ARG | A | 21 | 102.203 | 84.535 | 0.855 | 1.00 | 37.76 | N |
| ATOM | 43 | NH2 | ARG | A | 21 | 102.375 | 82.267 | 0.925 | 1.00 | 39.29 | N |
| ATOM | 44 | C | ARG | A | 21 | 103.790 | 78.970 | 5.828 | 1.00 | 14.63 | C |
| ATOM | 45 | O | ARG | A | 21 | 103.937 | 77.959 | 5.106 | 1.00 | 13.26 | O |
| ATOM | 46 | N | ALA | A | 22 | 103.657 | 78.927 | 7.153 | 1.00 | 13.38 | N |
| ATOM | 47 | CA | ALA | A | 22 | 103.252 | 77.712 | 7.863 | 1.00 | 13.12 | C |
| ATOM | 48 | CB | ALA | A | 22 | 103.228 | 77.979 | 9.355 | 1.00 | 13.15 | C |
| ATOM | 49 | C | ALA | A | 22 | 101.853 | 77.305 | 7.430 | 1.00 | 12.54 | C |
| ATOM | 50 | O | ALA | A | 22 | 101.048 | 78.137 | 7.152 | 1.00 | 10.93 | O |
| ATOM | 51 | N | ARG | A | 23 | 101.569 | 76.010 | 7.416 | 1.00 | 13.52 | N |
| ATOM | 52 | CA | ARG | A | 23 | 100.221 | 75.504 | 7.302 | 1.00 | 15.20 | C |
| ATOM | 53 | CB | ARG | A | 23 | 100.240 | 73.968 | 7.213 | 1.00 | 16.46 | C |
| ATOM | 54 | CG | ARG | A | 23 | 100.749 | 73.437 | 5.871 | 1.00 | 22.55 | C |
| ATOM | 55 | CD | ARG | A | 23 | 100.280 | 72.012 | 5.529 | 1.00 | 30.58 | C |
| ATOM | 56 | NE | ARG | A | 23 | 98.894 | 71.805 | 5.973 | 1.00 | 37.90 | N |
| ATOM | 57 | CZ | ARG | A | 23 | 97.800 | 72.337 | 5.406 | 1.00 | 41.96 | C |
| ATOM | 58 | NH1 | ARG | A | 23 | 97.877 | 73.105 | 4.310 | 1.00 | 44.79 | N |
| ATOM | 59 | NH2 | ARG | A | 23 | 96.610 | 72.092 | 5.950 | 1.00 | 42.74 | N |
| ATOM | 60 | C | ARG | A | 23 | 99.469 | 75.928 | 8.550 | 1.00 | 14.80 | C |
| ATOM | 61 | O | ARG | A | 23 | 100.069 | 75.959 | 9.613 | 1.00 | 13.03 | O |
| ATOM | 62 | N | PRO | A | 24 | 98.191 | 76.276 | 8.439 | 1.00 | 15.92 | N |
| ATOM | 63 | CA | PRO | A | 24 | 97.425 | 76.702 | 9.625 | 1.00 | 16.52 | C |
| ATOM | 64 | CB | PRO | A | 24 | 95.977 | 76.742 | 9.105 | 1.00 | 16.56 | C |
| ATOM | 65 | CG | PRO | A | 24 | 96.126 | 76.995 | 7.630 | 1.00 | 17.45 | C |
| ATOM | 66 | CD | PRO | A | 24 | 97.339 | 76.266 | 7.223 | 1.00 | 16.09 | C |
| ATOM | 67 | C | PRO | A | 24 | 97.576 | 75.702 | 10.790 | 1.00 | 15.95 | C |
| ATOM | 68 | O | PRO | A | 24 | 97.347 | 74.497 | 10.625 | 1.00 | 14.81 | O |
| ATOM | 69 | N | HIS | A | 25 | 98.032 | 76.199 | 11.943 | 1.00 | 15.42 | N |
| ATOM | 70 | CA | HIS | A | 25 | 98.109 | 75.382 | 13.165 | 1.00 | 15.53 | C |
| ATOM | 71 | CB | HIS | A | 25 | 96.686 | 74.992 | 13.634 | 1.00 | 15.68 | C |
| ATOM | 72 | CG | HIS | A | 25 | 95.803 | 76.185 | 13.760 | 1.00 | 17.38 | C |
| ATOM | 73 | ND1 | HIS | A | 25 | 94.971 | 76.601 | 12.745 | 1.00 | 21.68 | N |
| ATOM | 74 | CE1 | HIS | A | 25 | 94.366 | 77.716 | 13.108 | 1.00 | 18.86 | C |
| ATOM | 75 | NE2 | HIS | A | 25 | 94.799 | 78.050 | 14.306 | 1.00 | 18.20 | N |
| ATOM | 76 | CD2 | HIS | A | 25 | 95.703 | 77.117 | 14.732 | 1.00 | 16.13 | C |
| ATOM | 77 | C | HIS | A | 25 | 99.071 | 74.201 | 13.077 | 1.00 | 15.04 | C |
| ATOM | 78 | O | HIS | A | 25 | 98.982 | 73.246 | 13.854 | 1.00 | 15.01 | O |
| ATOM | 79 | N | ALA | A | 26 | 100.081 | 74.335 | 12.210 | 1.00 | 14.32 | N |
| ATOM | 80 | CA | ALA | A | 26 | 101.141 | 73.341 | 12.087 | 1.00 | 13.58 | C |
| ATOM | 81 | CB | ALA | A | 26 | 102.054 | 73.724 | 10.942 | 1.00 | 13.71 | C |
| ATOM | 82 | C | ALA | A | 26 | 101.957 | 73.228 | 13.360 | 1.00 | 13.81 | C |
| ATOM | 83 | O | ALA | A | 26 | 102.472 | 72.180 | 13.677 | 1.00 | 14.66 | O |
| ATOM | 84 | N | TRP | A | 27 | 102.112 | 74.343 | 14.070 | 1.00 | 12.65 | N |
| ATOM | 85 | CA | TRP | A | 27 | 102.961 | 74.411 | 15.242 | 1.00 | 12.44 | C |
| ATOM | 86 | CB | TRP | A | 27 | 104.164 | 75.324 | 14.950 | 1.00 | 11.90 | C |
| ATOM | 87 | CG | TRP | A | 27 | 104.779 | 74.998 | 13.658 | 1.00 | 11.95 | C |
| ATOM | 88 | CD1 | TRP | A | 27 | 104.822 | 75.779 | 12.561 | 1.00 | 14.66 | C |
| ATOM | 89 | NE1 | TRP | A | 27 | 105.430 | 75.115 | 11.524 | 1.00 | 16.97 | N |
| ATOM | 90 | CE2 | TRP | A | 27 | 105.796 | 73.871 | 11.953 | 1.00 | 18.72 | C |
| ATOM | 91 | CD2 | TRP | A | 27 | 105.385 | 73.758 | 13.292 | 1.00 | 16.86 | C |
| ATOM | 92 | CE3 | TRP | A | 27 | 105.649 | 72.558 | 13.974 | 1.00 | 18.50 | C |
| ATOM | 93 | CZ3 | TRP | A | 27 | 106.292 | 71.514 | 13.281 | 1.00 | 19.50 | C |
| ATOM | 94 | CH2 | TRP | A | 27 | 106.662 | 71.663 | 11.950 | 1.00 | 20.24 | C |
| ATOM | 95 | CZ2 | TRP | A | 27 | 106.410 | 72.820 | 11.261 | 1.00 | 20.48 | C |
| ATOM | 96 | C | TRP | A | 27 | 102.120 | 74.927 | 16.428 | 1.00 | 12.15 | C |
| ATOM | 97 | O | TRP | A | 27 | 102.139 | 76.123 | 16.728 | 1.00 | 10.87 | O |
| ATOM | 98 | N | PRO | A | 28 | 101.318 | 74.051 | 17.029 | 1.00 | 12.02 | N |
| ATOM | 99 | CA | PRO | A | 28 | 100.306 | 74.472 | 18.027 | 1.00 | 11.52 | C |
| ATOM | 100 | CB | PRO | A | 28 | 99.421 | 73.225 | 18.193 | 1.00 | 11.09 | C |
| ATOM | 101 | CG | PRO | A | 28 | 100.318 | 72.055 | 17.814 | 1.00 | 12.40 | C |
| ATOM | 102 | CD | PRO | A | 28 | 101.296 | 72.595 | 16.795 | 1.00 | 12.62 | C |
| ATOM | 103 | C | PRO | A | 28 | 100.844 | 74.974 | 19.379 | 1.00 | 10.89 | C |
| ATOM | 104 | O | PRO | A | 28 | 100.078 | 75.469 | 20.215 | 1.00 | 10.93 | O |
| ATOM | 105 | N | PHE | A | 29 | 102.151 | 74.870 | 19.571 | 1.00 | 10.01 | N |
| ATOM | 106 | CA | PHE | A | 29 | 102.836 | 75.450 | 20.715 | 1.00 | 9.68 | C |
| ATOM | 107 | CB | PHE | A | 29 | 104.056 | 74.588 | 21.049 | 1.00 | 10.65 | C |
| ATOM | 108 | CG | PHE | A | 29 | 104.944 | 74.299 | 19.852 | 1.00 | 11.29 | C |
| ATOM | 109 | CD1 | PHE | A | 29 | 105.829 | 75.279 | 19.366 | 1.00 | 9.14 | C |
| ATOM | 110 | CE1 | PHE | A | 29 | 106.618 | 75.027 | 18.232 | 1.00 | 12.47 | C |
| ATOM | 111 | CZ | PHE | A | 29 | 106.558 | 73.773 | 17.612 | 1.00 | 11.31 | C |
| ATOM | 112 | CE2 | PHE | A | 29 | 105.706 | 72.814 | 18.079 | 1.00 | 12.22 | C |
| ATOM | 113 | CD2 | PHE | A | 29 | 104.899 | 73.064 | 19.218 | 1.00 | 13.06 | C |
| ATOM | 114 | C | PHE | A | 29 | 103.262 | 76.912 | 20.466 | 1.00 | 8.39 | C |
| ATOM | 115 | O | PHE | A | 29 | 103.734 | 77.606 | 21.359 | 1.00 | 9.27 | O |
| ATOM | 116 | N | MET | A | 30 | 103.056 | 77.388 | 19.258 | 1.00 | 9.01 | N |
| ATOM | 117 | CA | MET | A | 30 | 103.509 | 78.731 | 18.853 | 1.00 | 8.83 | C |
| ATOM | 118 | CB | MET | A | 30 | 103.599 | 78.862 | 17.322 | 1.00 | 7.68 | C |
| ATOM | 119 | CG | MET | A | 30 | 104.156 | 80.203 | 16.827 | 1.00 | 8.55 | C |
| ATOM | 120 | SD | MET | A | 30 | 105.865 | 80.547 | 17.244 | 1.00 | 10.47 | S |
| ATOM | 121 | CE | MET | A | 30 | 105.936 | 82.390 | 17.192 | 1.00 | 10.55 | C |
| ATOM | 122 | C | MET | A | 30 | 102.592 | 79.810 | 19.420 | 1.00 | 9.35 | C |
| ATOM | 123 | O | MET | A | 30 | 101.357 | 79.770 | 19.261 | 1.00 | 9.16 | O |
| ATOM | 124 | N | VAL | A | 31 | 103.222 | 80.807 | 20.046 | 1.00 | 8.77 | N |
| ATOM | 125 | CA | VAL | A | 31 | 102.510 | 81.849 | 20.798 | 1.00 | 8.59 | C |
| ATOM | 126 | CB | VAL | A | 31 | 102.882 | 81.771 | 22.329 | 1.00 | 8.52 | C |
| ATOM | 127 | CG1 | VAL | A | 31 | 102.294 | 82.912 | 23.144 | 1.00 | 8.78 | C |
| ATOM | 128 | CG2 | VAL | A | 31 | 102.501 | 80.405 | 22.918 | 1.00 | 7.76 | C |
| ATOM | 129 | C | VAL | A | 31 | 102.888 | 83.238 | 20.271 | 1.00 | 9.28 | C |
| ATOM | 130 | O | VAL | A | 31 | 104.023 | 83.471 | 19.956 | 1.00 | 8.27 | O |
| ATOM | 131 | N | SER | A | 32 | 101.917 | 84.155 | 20.211 | 1.00 | 9.39 | N |
| ATOM | 132 | CA | SER | A | 32 | 102.154 | 85.572 | 19.943 | 1.00 | 9.16 | C |
| ATOM | 133 | CB | SER | A | 32 | 101.140 | 86.086 | 18.907 | 1.00 | 9.41 | C |
| ATOM | 134 | OG | SER | A | 32 | 101.267 | 87.467 | 18.652 | 1.00 | 9.14 | O |
| ATOM | 135 | C | SER | A | 32 | 101.992 | 86.373 | 21.224 | 1.00 | 9.70 | C |
| ATOM | 136 | O | SER | A | 32 | 100.929 | 86.353 | 21.840 | 1.00 | 9.35 | O |
| ATOM | 137 | N | LEU | A | 33 | 103.052 | 87.063 | 21.630 | 1.00 | 8.66 | N |
| ATOM | 138 | CA | LEU | A | 33 | 102.966 | 88.055 | 22.684 | 1.00 | 9.28 | C |
| ATOM | 139 | CB | LEU | A | 33 | 104.337 | 88.284 | 23.323 | 1.00 | 9.04 | C |
| ATOM | 140 | CG | LEU | A | 33 | 104.829 | 87.461 | 24.525 | 1.00 | 13.60 | C |
| ATOM | 141 | CD1 | LEU | A | 33 | 103.998 | 86.240 | 24.869 | 1.00 | 10.80 | C |
| ATOM | 142 | CD2 | LEU | A | 33 | 106.350 | 87.240 | 24.626 | 1.00 | 11.72 | C |
| ATOM | 143 | C | LEU | A | 33 | 102.525 | 89.348 | 22.039 | 1.00 | 9.67 | C |
| ATOM | 144 | O | LEU | A | 33 | 103.095 | 89.766 | 21.023 | 1.00 | 10.61 | O |
| ATOM | 145 | N | GLN | A | 34 | 101.574 | 90.015 | 22.659 | 1.00 | 9.90 | N |
| ATOM | 146 | CA | GLN | A | 34 | 100.934 | 91.180 | 22.075 | 1.00 | 10.83 | C |
| ATOM | 147 | CB | GLN | A | 34 | 99.552 | 90.774 | 21.514 | 1.00 | 10.37 | C |
| ATOM | 148 | CG | GLN | A | 34 | 99.683 | 89.644 | 20.465 | 1.00 | 10.21 | C |
| ATOM | 149 | CD | GLN | A | 34 | 98.533 | 89.527 | 19.474 | 1.00 | 11.06 | C |
| ATOM | 150 | OE1 | GLN | A | 34 | 98.662 | 88.812 | 18.443 | 1.00 | 12.16 | O |
| ATOM | 151 | NE2 | GLN | A | 34 | 97.433 | 90.229 | 19.738 | 1.00 | 6.95 | N |
| ATOM | 152 | C | GLN | A | 34 | 100.785 | 92.328 | 23.051 | 1.00 | 11.09 | C |
| ATOM | 153 | O | GLN | A | 34 | 100.607 | 92.134 | 24.271 | 1.00 | 11.42 | O |
| ATOM | 154 | N | LEU | A | 35 | 100.866 | 93.541 | 22.521 | 1.00 | 12.49 | N |
| ATOM | 155 | CA | LEU | A | 35 | 100.598 | 94.742 | 23.307 | 1.00 | 13.02 | C |
| ATOM | 156 | CB | LEU | A | 35 | 101.876 | 95.544 | 23.604 | 1.00 | 14.16 | C |
| ATOM | 157 | CG | LEU | A | 35 | 102.555 | 95.067 | 24.875 | 1.00 | 17.69 | C |
| ATOM | 158 | CD1 | LEU | A | 35 | 104.040 | 95.420 | 24.820 | 1.00 | 22.64 | C |
| ATOM | 159 | CD2 | LEU | A | 35 | 101.899 | 95.708 | 26.106 | 1.00 | 20.31 | C |
| ATOM | 160 | C | LEU | A | 35 | 99.607 | 95.564 | 22.500 | 1.00 | 13.02 | C |
| ATOM | 161 | O | LEU | A | 35 | 99.757 | 95.740 | 21.305 | 1.00 | 11.63 | O |
| ATOM | 162 | N | ARG | A | 36 | 98.554 | 96.005 | 23.160 | 1.00 | 14.36 | N |
| ATOM | 163 | CA | ARG | A | 36 | 97.490 | 96.757 | 22.500 | 1.00 | 15.22 | C |
| ATOM | 164 | CB | ARG | A | 36 | 98.002 | 98.174 | 22.233 | 1.00 | 16.26 | C |
| ATOM | 165 | CG | ARG | A | 36 | 98.413 | 98.879 | 23.514 | 1.00 | 20.56 | C |
| ATOM | 166 | CD | ARG | A | 36 | 98.639 | 100.377 | 23.373 | 1.00 | 26.34 | C |
| ATOM | 167 | NE | ARG | A | 36 | 99.218 | 100.993 | 24.584 | 1.00 | 32.72 | N |
| ATOM | 168 | CZ | ARG | A | 36 | 100.505 | 100.893 | 24.972 | 1.00 | 35.65 | C |
| ATOM | 169 | NH1 | ARG | A | 36 | 101.372 | 100.185 | 24.259 | 1.00 | 36.15 | N |
| ATOM | 170 | NH2 | ARG | A | 36 | 100.922 | 101.506 | 26.088 | 1.00 | 36.89 | N |
| ATOM | 171 | C | ARG | A | 36 | 96.943 | 96.043 | 21.270 | 1.00 | 14.12 | C |
| ATOM | 172 | O | ARG | A | 36 | 96.556 | 96.645 | 20.294 | 1.00 | 15.69 | O |
| ATOM | 173 | N | GLY | A | 38 | 96.846 | 94.729 | 21.359 | 1.00 | 13.55 | N |
| ATOM | 174 | CA | GLY | A | 38 | 96.262 | 93.921 | 20.309 | 1.00 | 12.72 | C |
| ATOM | 175 | C | GLY | A | 38 | 97.207 | 93.671 | 19.145 | 1.00 | 12.07 | C |
| ATOM | 176 | O | GLY | A | 38 | 96.775 | 93.241 | 18.102 | 1.00 | 12.31 | O |
| ATOM | 177 | N | GLY | A | 39 | 98.502 | 93.926 | 19.348 | 1.00 | 10.27 | N |
| ATOM | 178 | CA | GLY | A | 39 | 99.494 | 93.795 | 18.297 | 1.00 | 9.84 | C |
| ATOM | 179 | C | GLY | A | 39 | 100.675 | 92.907 | 18.700 | 1.00 | 8.47 | C |
| ATOM | 180 | O | GLY | A | 39 | 101.242 | 93.099 | 19.770 | 1.00 | 9.08 | O |
| ATOM | 181 | N | HIS | A | 40 | 101.036 | 91.990 | 17.818 | 1.00 | 9.12 | N |
| ATOM | 182 | CA | HIS | A | 40 | 102.189 | 91.104 | 17.955 | 1.00 | 9.28 | C |
| ATOM | 183 | CB | HIS | A | 40 | 102.346 | 90.193 | 16.715 | 1.00 | 8.88 | C |
| ATOM | 184 | CG | HIS | A | 40 | 103.573 | 89.327 | 16.776 | 1.00 | 9.92 | C |
| ATOM | 185 | ND1 | HIS | A | 40 | 104.822 | 89.764 | 16.364 | 1.00 | 13.25 | N |
| ATOM | 186 | CE1 | HIS | A | 40 | 105.716 | 88.805 | 16.583 | 1.00 | 8.38 | C |
| ATOM | 187 | NE2 | HIS | A | 40 | 105.088 | 87.771 | 17.125 | 1.00 | 12.66 | N |
| ATOM | 188 | CD2 | HIS | A | 40 | 103.753 | 88.076 | 17.260 | 1.00 | 7.56 | C |
| ATOM | 189 | C | HIS | A | 40 | 103.473 | 91.924 | 18.090 | 1.00 | 9.91 | C |
| ATOM | 190 | O | HIS | A | 40 | 103.686 | 92.834 | 17.267 | 1.00 | 10.50 | O |
| ATOM | 191 | N | PHE | A | 41 | 104.291 | 91.611 | 19.100 | 1.00 | 8.69 | N |
| ATOM | 192 | CA | PHE | A | 41 | 105.680 | 92.166 | 19.197 | 1.00 | 8.84 | C |
| ATOM | 193 | CB | PHE | A | 41 | 105.740 | 93.266 | 20.294 | 1.00 | 7.85 | C |
| ATOM | 194 | CG | PHE | A | 41 | 105.619 | 92.752 | 21.678 | 1.00 | 7.97 | C |
| ATOM | 195 | CD1 | PHE | A | 41 | 106.742 | 92.351 | 22.398 | 1.00 | 8.70 | C |
| ATOM | 196 | CE1 | PHE | A | 41 | 106.632 | 91.835 | 23.678 | 1.00 | 10.47 | C |
| ATOM | 197 | CZ | PHE | A | 41 | 105.387 | 91.743 | 24.292 | 1.00 | 8.91 | C |
| ATOM | 198 | CE2 | PHE | A | 41 | 104.251 | 92.157 | 23.585 | 1.00 | 6.58 | C |
| ATOM | 199 | CD2 | PHE | A | 41 | 104.382 | 92.671 | 22.285 | 1.00 | 4.33 | C |
| ATOM | 200 | C | PHE | A | 41 | 106.796 | 91.118 | 19.380 | 1.00 | 8.75 | C |
| ATOM | 201 | O | PHE | A | 41 | 107.973 | 91.404 | 19.177 | 1.00 | 7.89 | O |
| ATOM | 202 | N | CYS | A | 42 | 106.440 | 89.905 | 19.801 | 1.00 | 7.07 | N |
| ATOM | 203 | CA | CYS | A | 42 | 107.408 | 88.850 | 20.095 | 1.00 | 6.75 | C |
| ATOM | 204 | CB | CYS | A | 42 | 108.043 | 89.036 | 21.488 | 1.00 | 5.78 | C |
| ATOM | 205 | SG | CYS | A | 42 | 109.595 | 90.006 | 21.462 | 1.00 | 8.10 | S |
| ATOM | 206 | C | CYS | A | 42 | 106.726 | 87.486 | 20.037 | 1.00 | 6.84 | C |
| ATOM | 207 | O | CYS | A | 42 | 105.550 | 87.403 | 20.281 | 1.00 | 9.21 | O |
| ATOM | 208 | N | GLY | A | 43 | 107.472 | 86.443 | 19.699 | 1.00 | 7.15 | N |
| ATOM | 209 | CA | GLY | A | 43 | 107.022 | 85.078 | 19.820 | 1.00 | 6.81 | C |
| ATOM | 210 | C | GLY | A | 43 | 107.158 | 84.546 | 21.238 | 1.00 | 7.39 | C |
| ATOM | 211 | O | GLY | A | 43 | 107.810 | 85.163 | 22.102 | 1.00 | 8.22 | O |
| ATOM | 212 | N | ALA | A | 44 | 106.515 | 83.394 | 21.474 | 1.00 | 7.55 | N |
| ATOM | 213 | CA | ALA | A | 44 | 106.774 | 82.560 | 22.622 | 1.00 | 7.69 | C |
| ATOM | 214 | CB | ALA | A | 44 | 106.005 | 83.039 | 23.815 | 1.00 | 8.55 | C |
| ATOM | 215 | C | ALA | A | 44 | 106.409 | 81.112 | 22.315 | 1.00 | 8.90 | C |
| ATOM | 216 | O | ALA | A | 44 | 105.865 | 80.827 | 21.266 | 1.00 | 8.41 | O |
| ATOM | 217 | N | THR | A | 45 | 106.685 | 80.234 | 23.269 | 1.00 | 8.71 | N |
| ATOM | 218 | CA | THR | A | 45 | 106.371 | 78.805 | 23.158 | 1.00 | 8.71 | C |
| ATOM | 219 | CB | THR | A | 45 | 107.663 | 77.996 | 23.051 | 1.00 | 8.85 | C |
| ATOM | 220 | OG1 | THR | A | 45 | 108.434 | 78.453 | 21.942 | 1.00 | 9.89 | O |
| ATOM | 221 | CG2 | THR | A | 45 | 107.407 | 76.508 | 22.735 | 1.00 | 8.98 | C |
| ATOM | 222 | C | THR | A | 45 | 105.622 | 78.353 | 24.409 | 1.00 | 8.64 | C |
| ATOM | 223 | O | THR | A | 45 | 106.097 | 78.547 | 25.542 | 1.00 | 8.08 | O |
| ATOM | 224 | N | LEU | A | 46 | 104.504 | 77.673 | 24.203 | 1.00 | 9.20 | N |
| ATOM | 225 | CA | LEU | A | 46 | 103.744 | 77.028 | 25.276 | 1.00 | 9.79 | C |
| ATOM | 226 | CB | LEU | A | 46 | 102.345 | 76.661 | 24.784 | 1.00 | 9.74 | C |
| ATOM | 227 | CG | LEU | A | 46 | 101.324 | 76.241 | 25.846 | 1.00 | 11.02 | C |
| ATOM | 228 | CD1 | LEU | A | 46 | 100.998 | 77.443 | 26.756 | 1.00 | 11.30 | C |
| ATOM | 229 | CD2 | LEU | A | 46 | 100.093 | 75.720 | 25.189 | 1.00 | 11.93 | C |
| ATOM | 230 | C | LEU | A | 46 | 104.465 | 75.758 | 25.731 | 1.00 | 9.50 | C |
| ATOM | 231 | O | LEU | A | 46 | 104.741 | 74.852 | 24.918 | 1.00 | 8.95 | O |
| ATOM | 232 | N | ILE | A | 47 | 104.850 | 75.764 | 26.990 | 1.00 | 9.64 | N |
| ATOM | 233 | CA | ILE | A | 47 | 105.671 | 74.681 | 27.576 | 1.00 | 10.50 | C |
| ATOM | 234 | CB | ILE | A | 47 | 107.063 | 75.166 | 27.992 | 1.00 | 10.96 | C |
| ATOM | 235 | CG1 | ILE | A | 47 | 107.041 | 76.261 | 29.045 | 1.00 | 10.62 | C |
| ATOM | 236 | CD1 | ILE | A | 47 | 108.387 | 76.518 | 29.649 | 1.00 | 13.86 | C |
| ATOM | 237 | CG2 | ILE | A | 47 | 107.864 | 75.613 | 26.738 | 1.00 | 11.02 | C |
| ATOM | 238 | C | ILE | A | 47 | 104.946 | 73.910 | 28.707 | 1.00 | 11.31 | C |
| ATOM | 239 | O | ILE | A | 47 | 105.456 | 72.883 | 29.218 | 1.00 | 11.68 | O |
| ATOM | 240 | N | ALA | A | 48 | 103.792 | 74.439 | 29.084 | 1.00 | 10.59 | N |
| ATOM | 241 | CA | ALA | A | 48 | 102.837 | 73.805 | 30.014 | 1.00 | 11.81 | C |
| ATOM | 242 | CB | ALA | A | 48 | 103.303 | 73.930 | 31.432 | 1.00 | 12.78 | C |
| ATOM | 243 | C | ALA | A | 48 | 101.503 | 74.551 | 29.791 | 1.00 | 11.40 | C |
| ATOM | 244 | O | ALA | A | 48 | 101.501 | 75.575 | 29.136 | 1.00 | 10.40 | O |
| ATOM | 245 | N | PRO | A | 49 | 100.371 | 74.078 | 30.308 | 1.00 | 12.14 | N |
| ATOM | 246 | CA | PRO | A | 49 | 99.101 | 74.763 | 30.028 | 1.00 | 12.33 | C |
| ATOM | 247 | CB | PRO | A | 49 | 98.059 | 73.867 | 30.722 | 1.00 | 12.85 | C |
| ATOM | 248 | CG | PRO | A | 49 | 98.724 | 72.552 | 30.819 | 1.00 | 15.10 | C |
| ATOM | 249 | CD | PRO | A | 49 | 100.174 | 72.906 | 31.171 | 1.00 | 12.65 | C |
| ATOM | 250 | C | PRO | A | 49 | 99.079 | 76.185 | 30.591 | 1.00 | 12.27 | C |
| ATOM | 251 | O | PRO | A | 49 | 98.321 | 77.003 | 30.129 | 1.00 | 12.33 | O |
| ATOM | 252 | N | ASN | A | 50 | 99.890 | 76.449 | 31.613 | 1.00 | 11.03 | N |
| ATOM | 253 | CA | ASN | A | 50 | 99.898 | 77.747 | 32.271 | 1.00 | 10.83 | C |
| ATOM | 254 | CB | ASN | A | 50 | 99.343 | 77.647 | 33.715 | 1.00 | 11.55 | C |
| ATOM | 255 | CG | ASN | A | 50 | 100.136 | 76.718 | 34.575 | 1.00 | 12.76 | C |
| ATOM | 256 | OD1 | ASN | A | 50 | 100.794 | 75.810 | 34.064 | 1.00 | 13.46 | O |
| ATOM | 257 | ND2 | ASN | A | 50 | 100.124 | 76.949 | 35.896 | 1.00 | 11.54 | N |
| ATOM | 258 | C | ASN | A | 50 | 101.289 | 78.401 | 32.282 | 1.00 | 10.59 | C |
| ATOM | 259 | O | ASN | A | 50 | 101.556 | 79.240 | 33.122 | 1.00 | 11.10 | O |
| ATOM | 260 | N | PHE | A | 51 | 102.192 | 77.971 | 31.390 | 1.00 | 9.81 | N |
| ATOM | 261 | CA | PHE | A | 51 | 103.491 | 78.631 | 31.236 | 1.00 | 8.97 | C |
| ATOM | 262 | CB | PHE | A | 51 | 104.586 | 77.845 | 31.962 | 1.00 | 8.84 | C |
| ATOM | 263 | CG | PHE | A | 51 | 104.513 | 77.857 | 33.475 | 1.00 | 7.85 | C |
| ATOM | 264 | CD1 | PHE | A | 51 | 105.200 | 78.775 | 34.225 | 1.00 | 8.30 | C |
| ATOM | 265 | CE1 | PHE | A | 51 | 105.159 | 78.723 | 35.611 | 1.00 | 8.61 | C |
| ATOM | 266 | CZ | PHE | A | 51 | 104.435 | 77.761 | 36.265 | 1.00 | 10.58 | C |
| ATOM | 267 | CE2 | PHE | A | 51 | 103.746 | 76.844 | 35.553 | 1.00 | 9.43 | C |
| ATOM | 268 | CD2 | PHE | A | 51 | 103.798 | 76.861 | 34.152 | 1.00 | 9.24 | C |
| ATOM | 269 | C | PHE | A | 51 | 103.903 | 78.746 | 29.752 | 1.00 | 8.21 | C |
| ATOM | 270 | O | PHE | A | 51 | 103.693 | 77.826 | 28.947 | 1.00 | 8.67 | O |
| ATOM | 271 | N | VAL | A | 52 | 104.507 | 79.876 | 29.398 | 1.00 | 9.27 | N |
| ATOM | 272 | CA | VAL | A | 52 | 105.230 | 80.006 | 28.138 | 1.00 | 8.69 | C |
| ATOM | 273 | CB | VAL | A | 52 | 104.555 | 81.020 | 27.162 | 1.00 | 9.28 | C |
| ATOM | 274 | CG1 | VAL | A | 52 | 104.665 | 82.427 | 27.637 | 1.00 | 8.65 | C |
| ATOM | 275 | CG2 | VAL | A | 52 | 103.126 | 80.647 | 26.865 | 1.00 | 8.69 | C |
| ATOM | 276 | C | VAL | A | 52 | 106.654 | 80.451 | 28.428 | 1.00 | 9.21 | C |
| ATOM | 277 | O | VAL | A | 52 | 106.947 | 80.946 | 29.538 | 1.00 | 9.12 | O |
| ATOM | 278 | N | MET | A | 53 | 107.546 | 80.232 | 27.450 | 1.00 | 8.51 | N |
| ATOM | 279 | CA | MET | A | 53 | 108.915 | 80.739 | 27.524 | 1.00 | 8.18 | C |
| ATOM | 280 | CB | MET | A | 53 | 109.993 | 79.626 | 27.581 | 1.00 | 8.71 | C |
| ATOM | 281 | CG | MET | A | 53 | 110.054 | 78.754 | 26.421 | 1.00 | 9.83 | C |
| ATOM | 282 | SD | MET | A | 53 | 111.305 | 77.447 | 26.725 | 1.00 | 12.03 | S |
| ATOM | 283 | CE | MET | A | 53 | 111.302 | 76.718 | 25.205 | 1.00 | 10.11 | C |
| ATOM | 284 | C | MET | A | 53 | 109.102 | 81.605 | 26.307 | 1.00 | 8.20 | C |
| ATOM | 285 | O | MET | A | 53 | 108.548 | 81.331 | 25.264 | 1.00 | 8.12 | O |
| ATOM | 286 | N | SER | A | 54 | 109.893 | 82.650 | 26.483 | 1.00 | 8.11 | N |
| ATOM | 287 | CA | SER | A | 54 | 110.170 | 83.613 | 25.461 | 1.00 | 8.55 | C |
| ATOM | 288 | CB | SER | A | 54 | 109.099 | 84.739 | 25.495 | 1.00 | 6.88 | C |
| ATOM | 289 | OG | SER | A | 54 | 109.309 | 85.679 | 24.460 | 1.00 | 5.93 | O |
| ATOM | 290 | C | SER | A | 54 | 111.578 | 84.163 | 25.723 | 1.00 | 8.48 | C |
| ATOM | 291 | O | SER | A | 54 | 112.340 | 83.613 | 26.572 | 1.00 | 10.63 | O |
| ATOM | 292 | N | ALA | A | 55 | 111.949 | 85.185 | 24.986 | 1.00 | 8.91 | N |
| ATOM | 293 | CA | ALA | A | 55 | 113.213 | 85.909 | 25.230 | 1.00 | 8.92 | C |
| ATOM | 294 | CB | ALA | A | 55 | 113.725 | 86.547 | 23.954 | 1.00 | 10.13 | C |
| ATOM | 295 | C | ALA | A | 55 | 113.035 | 86.959 | 26.322 | 1.00 | 9.97 | C |
| ATOM | 296 | O | ALA | A | 55 | 112.076 | 87.735 | 26.318 | 1.00 | 9.69 | O |
| ATOM | 297 | N | ALA | A | 56 | 113.950 | 86.981 | 27.289 | 1.00 | 9.38 | N |
| ATOM | 298 | CA | ALA | A | 56 | 113.861 | 87.962 | 28.336 | 1.00 | 9.73 | C |
| ATOM | 299 | CB | ALA | A | 56 | 114.988 | 87.809 | 29.362 | 1.00 | 11.15 | C |
| ATOM | 300 | C | ALA | A | 56 | 113.827 | 89.387 | 27.776 | 1.00 | 10.00 | C |
| ATOM | 301 | O | ALA | A | 56 | 113.151 | 90.239 | 28.363 | 1.00 | 9.87 | O |
| ATOM | 302 | N | HIS | A | 57 | 114.476 | 89.662 | 26.640 | 1.00 | 8.88 | N |
| ATOM | 303 | CA | HIS | A | 57 | 114.542 | 91.051 | 26.192 | 1.00 | 11.29 | C |
| ATOM | 304 | CB | HIS | A | 57 | 115.614 | 91.251 | 25.119 | 1.00 | 10.99 | C |
| ATOM | 305 | CG | HIS | A | 57 | 115.195 | 90.887 | 23.718 | 1.00 | 10.61 | C |
| ATOM | 306 | ND1 | HIS | A | 57 | 115.912 | 89.999 | 22.949 | 1.00 | 10.71 | N |
| ATOM | 307 | CE1 | HIS | A | 57 | 115.411 | 89.959 | 21.738 | 1.00 | 10.35 | C |
| ATOM | 308 | NE2 | HIS | A | 57 | 114.374 | 90.757 | 21.695 | 1.00 | 10.85 | N |
| ATOM | 309 | CD2 | HIS | A | 57 | 114.165 | 91.293 | 22.939 | 1.00 | 14.41 | C |
| ATOM | 310 | C | HIS | A | 57 | 113.177 | 91.497 | 25.670 | 1.00 | 12.20 | C |
| ATOM | 311 | O | HIS | A | 57 | 112.913 | 92.667 | 25.588 | 1.00 | 13.34 | O |
| ATOM | 312 | N | CYS | A | 58 | 112.323 | 90.534 | 25.348 | 1.00 | 12.07 | N |
| ATOM | 313 | CA | CYS | A | 58 | 110.976 | 90.808 | 24.890 | 1.00 | 12.64 | C |
| ATOM | 314 | CB | CYS | A | 58 | 110.291 | 89.523 | 24.379 | 1.00 | 11.38 | C |
| ATOM | 315 | SG | CYS | A | 58 | 110.839 | 89.019 | 22.756 | 1.00 | 10.53 | S |
| ATOM | 316 | C | CYS | A | 58 | 110.126 | 91.455 | 25.978 | 1.00 | 13.41 | C |
| ATOM | 317 | O | CYS | A | 58 | 109.241 | 92.264 | 25.641 | 1.00 | 14.81 | O |
| ATOM | 318 | N | VAL | A | 59 | 110.387 | 91.158 | 27.251 | 1.00 | 12.35 | N |
| ATOM | 319 | CA | VAL | A | 59 | 109.594 | 91.765 | 28.334 | 1.00 | 13.90 | C |
| ATOM | 320 | CB | VAL | A | 59 | 108.926 | 90.718 | 29.222 | 1.00 | 13.68 | C |
| ATOM | 321 | CG1 | VAL | A | 59 | 108.024 | 89.904 | 28.370 | 1.00 | 13.22 | C |
| ATOM | 322 | CG2 | VAL | A | 59 | 109.947 | 89.799 | 29.995 | 1.00 | 14.50 | C |
| ATOM | 323 | C | VAL | A | 59 | 110.316 | 92.808 | 29.166 | 1.00 | 14.67 | C |
| ATOM | 324 | O | VAL | A | 59 | 109.813 | 93.229 | 30.194 | 1.00 | 14.72 | O |
| ATOM | 325 | N | ALA | A | 60 | 111.463 | 93.267 | 28.690 | 1.00 | 16.18 | N |
| ATOM | 326 | CA | ALA | A | 60 | 112.303 | 94.173 | 29.477 | 1.00 | 18.20 | C |
| ATOM | 327 | CB | ALA | A | 60 | 113.676 | 94.204 | 28.892 | 1.00 | 18.84 | C |
| ATOM | 328 | C | ALA | A | 60 | 111.806 | 95.605 | 29.615 | 1.00 | 20.18 | C |
| ATOM | 329 | O | ALA | A | 60 | 111.921 | 96.225 | 30.689 | 1.00 | 22.24 | O |
| ATOM | 330 | N | ASM | A | 61 | 111.352 | 96.227 | 28.557 | 1.00 | 20.16 | N |
| ATOM | 331 | CA | ASN | A | 61 | 111.070 | 97.655 | 28.811 | 1.00 | 21.16 | C |
| ATOM | 332 | CB | ASN | A | 61 | 112.085 | 98.534 | 28.034 | 1.00 | 21.70 | C |
| ATOM | 333 | CG | ASN | A | 61 | 112.053 | 100.026 | 28.427 | 1.00 | 23.36 | C |
| ATOM | 334 | OD1 | ASN | A | 61 | 111.457 | 100.435 | 29.419 | 1.00 | 24.97 | O |
| ATOM | 335 | ND2 | ASN | A | 61 | 112.695 | 100.841 | 27.602 | 1.00 | 27.59 | N |
| ATOM | 336 | C | ASN | A | 61 | 109.646 | 97.872 | 28.409 | 1.00 | 20.54 | C |
| ATOM | 337 | O | ASN | A | 61 | 109.363 | 98.722 | 27.598 | 1.00 | 20.13 | O |
| ATOM | 338 | N | VAL | A | 62 | 108.774 | 96.994 | 28.929 | 1.00 | 20.27 | N |
| ATOM | 339 | CA | VAL | A | 62 | 107.337 | 97.025 | 28.603 | 1.00 | 19.72 | C |
| ATOM | 340 | CB | VAL | A | 62 | 106.943 | 95.971 | 27.544 | 1.00 | 19.72 | C |
| ATOM | 341 | CG1 | VAL | A | 62 | 107.933 | 95.990 | 26.374 | 1.00 | 22.99 | C |
| ATOM | 342 | CG2 | VAL | A | 62 | 106.873 | 94.561 | 28.143 | 1.00 | 21.75 | C |
| ATOM | 343 | C | VAL | A | 62 | 106.532 | 96.857 | 29.864 | 1.00 | 18.11 | C |
| ATOM | 344 | O | VAL | A | 62 | 107.033 | 96.342 | 30.882 | 1.00 | 19.09 | O |
| ATOM | 345 | N | ASN | A | 62A | 105.292 | 97.303 | 29.825 | 1.00 | 16.69 | N |
| ATOM | 346 | CA | ASN | A | 62A | 104.343 | 97.071 | 30.910 | 1.00 | 16.12 | C |
| ATOM | 347 | CB | ASN | A | 62A | 103.134 | 98.018 | 30.823 | 1.00 | 16.42 | C |
| ATOM | 348 | CG | ASN | A | 62A | 102.237 | 97.893 | 32.015 | 1.00 | 15.36 | C |
| ATOM | 349 | OD1 | ASN | A | 62A | 102.487 | 97.075 | 32.899 | 1.00 | 17.04 | O |
| ATOM | 350 | ND2 | ASN | A | 62A | 101.215 | 98.746 | 32.094 | 1.00 | 19.57 | N |
| ATOM | 351 | C | ASN | A | 62A | 103.853 | 95.635 | 30.833 | 1.00 | 15.45 | C |
| ATOM | 352 | O | ASN | A | 62A | 102.909 | 95.335 | 30.119 | 1.00 | 14.69 | O |
| ATOM | 353 | N | VAL | A | 62B | 104.472 | 94.739 | 31.589 | 1.00 | 16.02 | N |
| ATOM | 354 | CA | VAL | A | 62B | 104.107 | 93.328 | 31.526 | 1.00 | 16.64 | C |
| ATOM | 355 | CB | VAL | A | 62B | 105.090 | 92.477 | 32.332 | 1.00 | 16.97 | C |
| ATOM | 356 | CG1 | VAL | A | 62B | 104.588 | 91.045 | 32.436 | 1.00 | 19.63 | C |
| ATOM | 357 | CG2 | VAL | A | 62B | 106.423 | 92.503 | 31.635 | 1.00 | 17.87 | C |
| ATOM | 358 | C | VAL | A | 62B | 102.634 | 93.068 | 31.920 | 1.00 | 16.68 | C |
| ATOM | 359 | O | VAL | A | 62B | 101.998 | 92.154 | 31.419 | 1.00 | 15.17 | O |
| ATOM | 360 | N | ARG | A | 63 | 102.087 | 93.918 | 32.778 | 1.00 | 16.66 | N |
| ATOM | 361 | CA | ARG | A | 63 | 100.674 | 93.868 | 33.097 | 1.00 | 17.74 | C |
| ATOM | 362 | CB | ARG | A | 63 | 100.337 | 94.975 | 34.093 | 1.00 | 18.58 | C |
| ATOM | 363 | CG | ARG | A | 63 | 100.889 | 94.794 | 35.486 | 1.00 | 23.92 | C |
| ATOM | 364 | CD | ARG | A | 63 | 100.384 | 95.889 | 36.467 | 1.00 | 30.06 | C |
| ATOM | 365 | NE | ARG | A | 63 | 100.566 | 97.216 | 35.889 | 1.00 | 34.06 | N |
| ATOM | 366 | CZ | ARG | A | 63 | 101.617 | 98.022 | 36.090 | 1.00 | 39.28 | C |
| ATOM | 367 | NH1 | ARG | A | 63 | 102.619 | 97.662 | 36.894 | 1.00 | 39.93 | N |
| ATOM | 368 | NH2 | ARG | A | 63 | 101.652 | 99.220 | 35.483 | 1.00 | 40.14 | N |
| ATOM | 369 | C | ARG | A | 63 | 99.729 | 93.998 | 31.884 | 1.00 | 16.25 | C |
| ATOM | 370 | O | ARG | A | 63 | 98.578 | 93.523 | 31.946 | 1.00 | 16.28 | O |
| ATOM | 371 | N | ALA | A | 64 | 100.197 | 94.644 | 30.819 | 1.00 | 15.21 | N |
| ATOM | 372 | CA | ALA | A | 64 | 99.439 | 94.885 | 29.604 | 1.00 | 15.53 | C |
| ATOM | 373 | CB | ALA | A | 64 | 99.731 | 96.263 | 29.063 | 1.00 | 16.35 | C |
| ATOM | 374 | C | ALA | A | 64 | 99.705 | 93.838 | 28.518 | 1.00 | 15.35 | C |
| ATOM | 375 | O | ALA | A | 64 | 99.020 | 93.823 | 27.496 | 1.00 | 14.43 | O |
| ATOM | 376 | N | VAL | A | 65 | 100.680 | 92.957 | 28.743 | 1.00 | 14.68 | N |
| ATOM | 377 | CA | VAL | A | 65 | 100.985 | 91.902 | 27.777 | 1.00 | 14.66 | C |
| ATOM | 378 | CB | VAL | A | 65 | 102.358 | 91.285 | 28.019 | 1.00 | 14.01 | C |
| ATOM | 379 | CG1 | VAL | A | 65 | 102.578 | 90.085 | 27.129 | 1.00 | 12.91 | C |
| ATOM | 380 | CG2 | VAL | A | 65 | 103.441 | 92.334 | 27.803 | 1.00 | 15.00 | C |
| ATOM | 381 | C | VAL | A | 65 | 99.887 | 90.839 | 27.723 | 1.00 | 14.62 | C |
| ATOM | 382 | O | VAL | A | 65 | 99.411 | 90.359 | 28.741 | 1.00 | 16.58 | O |
| ATOM | 383 | N | ARG | A | 65A | 99.482 | 90.510 | 26.512 | 1.00 | 14.38 | N |
| ATOM | 384 | CA | ARG | A | 65A | 98.558 | 89.433 | 26.243 | 1.00 | 14.33 | C |
| ATOM | 385 | CB | ARG | A | 65A | 97.358 | 89.918 | 25.428 | 1.00 | 15.27 | C |
| ATOM | 386 | CG | ARG | A | 65A | 96.471 | 90.982 | 26.179 | 1.00 | 18.57 | C |
| ATOM | 387 | CD | ARG | A | 65A | 95.889 | 90.465 | 27.530 | 1.00 | 25.87 | C |
| ATOM | 388 | NE | ARG | A | 65A | 95.164 | 91.427 | 28.401 | 1.00 | 28.60 | N |
| ATOM | 389 | CZ | ARG | A | 65A | 95.690 | 92.060 | 29.469 | 1.00 | 30.26 | C |
| ATOM | 390 | NH1 | ARG | A | 65A | 96.961 | 91.892 | 29.805 | 1.00 | 26.64 | N |
| ATOM | 391 | NH2 | ARG | A | 65A | 94.941 | 92.888 | 30.203 | 1.00 | 31.60 | N |
| ATOM | 392 | C | ARG | A | 65A | 99.318 | 88.274 | 25.587 | 1.00 | 13.66 | C |
| ATOM | 393 | O | ARG | A | 65A | 100.200 | 88.468 | 24.722 | 1.00 | 12.81 | O |
| ATOM | 394 | N | VAL | A | 66 | 99.016 | 87.077 | 26.074 | 1.00 | 13.00 | N |
| ATOM | 395 | CA | VAL | A | 66 | 99.635 | 85.868 | 25.596 | 1.00 | 12.73 | C |
| ATOM | 396 | CB | VAL | A | 66 | 100.093 | 84.984 | 26.774 | 1.00 | 11.57 | C |
| ATOM | 397 | CG1 | VAL | A | 66 | 100.897 | 83.803 | 26.251 | 1.00 | 13.25 | C |
| ATOM | 398 | CG2 | VAL | A | 66 | 100.938 | 85.775 | 27.770 | 1.00 | 15.52 | C |
| ATOM | 399 | C | VAL | A | 66 | 98.579 | 85.177 | 24.726 | 1.00 | 10.56 | C |
| ATOM | 400 | O | VAL | A | 66 | 97.596 | 84.709 | 25.244 | 1.00 | 11.38 | O |
| ATOM | 401 | N | VAL | A | 67 | 98.796 | 85.133 | 23.423 | 1.00 | 10.75 | N |
| ATOM | 402 | CA | VAL | A | 67 | 97.820 | 84.656 | 22.457 | 1.00 | 10.78 | C |
| ATOM | 403 | CB | VAL | A | 67 | 97.667 | 85.639 | 21.289 | 1.00 | 11.30 | C |
| ATOM | 404 | CG1 | VAL | A | 67 | 96.640 | 85.141 | 20.300 | 1.00 | 11.45 | C |
| ATOM | 405 | CG2 | VAL | A | 67 | 97.303 | 87.033 | 21.825 | 1.00 | 11.36 | C |
| ATOM | 406 | C | VAL | A | 67 | 98.249 | 83.309 | 21.930 | 1.00 | 10.70 | C |
| ATOM | 407 | O | VAL | A | 67 | 99.263 | 83.162 | 21.195 | 1.00 | 9.53 | O |
| ATOM | 408 | N | LEU | A | 68 | 97.491 | 82.304 | 22.326 | 1.00 | 11.29 | N |
| ATOM | 409 | CA | LEU | A | 68 | 97.725 | 80.926 | 21.880 | 1.00 | 10.82 | C |
| ATOM | 410 | CB | LEU | A | 68 | 97.403 | 79.960 | 23.015 | 1.00 | 11.32 | C |
| ATOM | 411 | CG | LEU | A | 68 | 97.881 | 80.252 | 24.442 | 1.00 | 11.69 | C |
| ATOM | 412 | CD1 | LEU | A | 68 | 97.465 | 79.109 | 25.377 | 1.00 | 10.63 | C |
| ATOM | 413 | CD2 | LEU | A | 68 | 99.336 | 80.414 | 24.544 | 1.00 | 10.03 | C |
| ATOM | 414 | C | LEU | A | 68 | 96.778 | 80.607 | 20.721 | 1.00 | 11.70 | C |
| ATOM | 415 | O | LEU | A | 68 | 95.747 | 81.250 | 20.584 | 1.00 | 11.54 | O |
| ATOM | 416 | N | GLY | A | 69 | 97.118 | 79.614 | 19.901 | 1.00 | 11.10 | N |
| ATOM | 417 | CA | GLY | A | 69 | 96.226 | 79.160 | 18.845 | 1.00 | 11.54 | C |
| ATOM | 418 | C | GLY | A | 69 | 96.075 | 80.062 | 17.632 | 1.00 | 10.81 | C |
| ATOM | 419 | O | GLY | A | 69 | 95.132 | 79.908 | 16.878 | 1.00 | 11.74 | O |
| ATOM | 420 | N | ALA | A | 70 | 96.974 | 81.024 | 17.452 | 1.00 | 10.68 | N |
| ATOM | 421 | CA | ALA | A | 70 | 96.859 | 81.990 | 16.366 | 1.00 | 10.77 | C |
| ATOM | 422 | CB | ALA | A | 70 | 97.474 | 83.334 | 16.753 | 1.00 | 11.86 | C |
| ATOM | 423 | C | ALA | A | 70 | 97.549 | 81.486 | 15.107 | 1.00 | 10.48 | C |
| ATOM | 424 | O | ALA | A | 70 | 98.477 | 80.641 | 15.159 | 1.00 | 11.17 | O |
| ATOM | 425 | N | HIS | A | 71 | 97.132 | 82.041 | 13.980 | 1.00 | 9.92 | N |
| ATOM | 426 | CA | HIS | A | 71 | 97.764 | 81.758 | 12.695 | 1.00 | 10.61 | C |
| ATOM | 427 | CB | HIS | A | 71 | 96.958 | 80.816 | 11.861 | 1.00 | 11.04 | C |
| ATOM | 428 | CG | HIS | A | 71 | 97.657 | 80.422 | 10.603 | 1.00 | 11.57 | C |
| ATOM | 429 | ND1 | HIS | A | 71 | 98.822 | 79.687 | 10.609 | 1.00 | 13.54 | N |
| ATOM | 430 | CE1 | HIS | A | 71 | 99.209 | 79.489 | 9.359 | 1.00 | 15.01 | C |
| ATOM | 431 | NE2 | HIS | A | 71 | 98.367 | 80.111 | 8.549 | 1.00 | 12.01 | N |
| ATOM | 432 | CD2 | HIS | A | 71 | 97.402 | 80.729 | 9.306 | 1.00 | 15.57 | C |
| ATOM | 433 | C | HIS | A | 71 | 97.961 | 83.027 | 11.868 | 1.00 | 10.53 | C |
| ATOM | 434 | O | HIS | A | 71 | 99.064 | 83.332 | 11.467 | 1.00 | 9.71 | O |
| ATOM | 435 | N | ASN | A | 72 | 96.856 | 83.719 | 11.614 | 1.00 | 11.16 | N |
| ATOM | 436 | CA | ASN | A | 72 | 96.813 | 84.932 | 10.812 | 1.00 | 11.81 | C |
| ATOM | 437 | CB | ASN | A | 72 | 95.800 | 84.778 | 9.672 | 1.00 | 12.85 | C |
| ATOM | 438 | CG | ASN | A | 72 | 95.899 | 85.901 | 8.634 | 1.00 | 12.77 | C |
| ATOM | 439 | OD1 | ASN | A | 72 | 96.200 | 87.048 | 8.972 | 1.00 | 15.35 | O |
| ATOM | 440 | ND2 | ASN | A | 72 | 95.585 | 85.582 | 7.383 | 1.00 | 12.61 | N |
| ATOM | 441 | C | ASN | A | 72 | 96.462 | 86.113 | 11.700 | 1.00 | 11.87 | C |
| ATOM | 442 | O | ASN | A | 72 | 95.352 | 86.238 | 12.179 | 1.00 | 11.62 | O |
| ATOM | 443 | N | LEU | A | 73 | 97.464 | 86.953 | 11.954 | 1.00 | 11.00 | N |
| ATOM | 444 | CA | LEU | A | 73 | 97.320 | 88.143 | 12.785 | 1.00 | 12.96 | C |
| ATOM | 445 | CB | LEU | A | 73 | 98.663 | 88.896 | 12.834 | 1.00 | 12.16 | C |
| ATOM | 446 | CG | LEU | A | 73 | 99.700 | 88.636 | 13.943 | 1.00 | 14.61 | C |
| ATOM | 447 | CD1 | LEU | A | 73 | 99.414 | 87.461 | 14.919 | 1.00 | 12.52 | C |
| ATOM | 448 | CD2 | LEU | A | 73 | 101.147 | 88.736 | 13.473 | 1.00 | 14.47 | C |
| ATOM | 449 | C | LEU | A | 73 | 96.289 | 89.146 | 12.272 | 1.00 | 13.92 | C |
| ATOM | 450 | O | LEU | A | 73 | 95.833 | 89.978 | 13.024 | 1.00 | 15.12 | O |
| ATOM | 451 | N | SER | A | 74 | 95.984 | 89.100 | 10.986 | 1.00 | 15.70 | N |
| ATOM | 452 | CA | SER | A | 74 | 94.978 | 89.987 | 10.403 | 1.00 | 16.25 | C |
| ATOM | 453 | CB | SER | A | 74 | 95.241 | 90.126 | 8.915 | 1.00 | 15.99 | C |
| ATOM | 454 | OG | SER | A | 74 | 96.388 | 90.912 | 8.742 | 1.00 | 18.53 | O |
| ATOM | 455 | C | SER | A | 74 | 93.539 | 89.537 | 10.599 | 1.00 | 18.54 | C |
| ATOM | 456 | O | SER | A | 74 | 92.616 | 90.310 | 10.291 | 1.00 | 18.61 | O |
| ATOM | 457 | N | ARG | A | 75 | 93.353 | 88.289 | 11.044 | 1.00 | 19.47 | N |
| ATOM | 458 | CA | ARG | A | 75 | 92.039 | 87.697 | 11.204 | 1.00 | 21.94 | C |
| ATOM | 459 | CB | ARG | A | 75 | 92.001 | 86.283 | 10.566 | 1.00 | 22.68 | C |
| ATOM | 460 | CG | ARG | A | 75 | 92.249 | 86.230 | 9.069 | 1.00 | 28.00 | C |
| ATOM | 461 | CD | ARG | A | 75 | 91.255 | 85.349 | 8.291 | 1.00 | 35.13 | C |
| ATOM | 462 | NE | ARG | A | 75 | 91.660 | 85.079 | 6.906 | 1.00 | 41.25 | N |
| ATOM | 463 | CZ | ARG | A | 75 | 91.716 | 85.983 | 5.918 | 1.00 | 46.60 | C |
| ATOM | 464 | NH1 | ARG | A | 75 | 91.411 | 87.266 | 6.128 | 1.00 | 48.48 | N |
| ATOM | 465 | NH2 | ARG | A | 75 | 92.107 | 85.605 | 4.700 | 1.00 | 49.26 | N |
| ATOM | 466 | C | ARG | A | 75 | 91.633 | 87.613 | 12.683 | 1.00 | 22.25 | C |
| ATOM | 467 | O | ARG | A | 75 | 92.479 | 87.596 | 13.576 | 1.00 | 21.90 | O |
| ATOM | 468 | N | ARG | A | 76 | 90.336 | 87.484 | 12.938 | 1.00 | 23.14 | N |
| ATOM | 469 | CA | ARG | A | 76 | 89.820 | 87.526 | 14.320 | 1.00 | 24.97 | C |
| ATOM | 470 | CB | ARG | A | 76 | 88.319 | 87.941 | 14.358 | 1.00 | 25.90 | C |
| ATOM | 471 | CG | ARG | A | 76 | 87.512 | 87.804 | 13.061 | 1.00 | 30.79 | C |
| ATOM | 472 | CD | ARG | A | 76 | 87.979 | 88.655 | 11.827 | 1.00 | 36.80 | C |
| ATOM | 473 | NE | ARG | A | 76 | 88.182 | 87.786 | 10.654 | 1.00 | 39.26 | N |
| ATOM | 474 | CZ | ARG | A | 76 | 88.388 | 88.196 | 9.420 | 1.00 | 41.79 | C |
| ATOM | 475 | NH1 | ARG | A | 76 | 88.454 | 89.495 | 9.137 | 1.00 | 43.84 | N |
| ATOM | 476 | NH2 | ARG | A | 76 | 88.524 | 87.292 | 8.455 | 1.00 | 43.22 | N |
| ATOM | 477 | C | ARG | A | 76 | 90.102 | 86.250 | 15.199 | 1.00 | 24.33 | C |
| ATOM | 478 | O | ARG | A | 76 | 90.398 | 86.372 | 16.424 | 1.00 | 26.61 | O |
| ATOM | 479 | N | GLU | A | 77 | 90.006 | 85.093 | 14.546 | 1.00 | 22.60 | N |
| ATOM | 480 | CA | GLU | A | 77 | 90.370 | 83.718 | 14.969 | 1.00 | 21.49 | C |
| ATOM | 481 | CB | GLU | A | 77 | 91.897 | 83.494 | 14.926 | 1.00 | 21.24 | C |
| ATOM | 482 | CG | GLU | A | 77 | 92.522 | 83.820 | 13.593 | 1.00 | 18.65 | C |
| ATOM | 483 | CD | GLU | A | 77 | 93.972 | 83.365 | 13.466 | 1.00 | 15.91 | C |
| ATOM | 484 | OE1 | GLU | A | 77 | 94.348 | 82.806 | 12.404 | 1.00 | 12.06 | O |
| ATOM | 485 | OE2 | GLU | A | 77 | 94.740 | 83.578 | 14.424 | 1.00 | 11.02 | O |
| ATOM | 486 | C | GLU | A | 77 | 89.787 | 83.097 | 16.254 | 1.00 | 21.88 | C |
| ATOM | 487 | O | GLU | A | 77 | 90.365 | 83.271 | 17.333 | 1.00 | 20.39 | O |
| ATOM | 488 | N | PRO | A | 78 | 88.675 | 82.355 | 16.137 | 1.00 | 22.69 | N |
| ATOM | 489 | CA | PRO | A | 78 | 88.085 | 81.612 | 17.280 | 1.00 | 22.59 | C |
| ATOM | 490 | CB | PRO | A | 78 | 86.822 | 80.953 | 16.676 | 1.00 | 22.63 | C |
| ATOM | 491 | CG | PRO | A | 78 | 86.475 | 81.816 | 15.523 | 1.00 | 23.75 | C |
| ATOM | 492 | CD | PRO | A | 78 | 87.811 | 82.247 | 14.937 | 1.00 | 23.66 | C |
| ATOM | 493 | C | PRO | A | 78 | 88.939 | 80.529 | 17.921 | 1.00 | 21.71 | C |
| ATOM | 494 | O | PRO | A | 78 | 88.655 | 80.204 | 19.066 | 1.00 | 22.88 | O |
| ATOM | 495 | N | THR | A | 79 | 89.918 | 79.983 | 17.211 | 1.00 | 20.18 | N |
| ATOM | 496 | CA | THR | A | 79 | 90.874 | 79.051 | 17.787 | 1.00 | 20.03 | C |
| ATOM | 497 | CB | THR | A | 79 | 91.791 | 78.457 | 16.685 | 1.00 | 20.60 | C |
| ATOM | 498 | OG1 | THR | A | 79 | 92.467 | 79.509 | 15.970 | 1.00 | 20.27 | O |
| ATOM | 499 | CG2 | THR | A | 79 | 90.993 | 77.670 | 15.620 | 1.00 | 22.56 | C |
| ATOM | 500 | C | THR | A | 79 | 91.793 | 79.669 | 18.874 | 1.00 | 18.52 | C |
| ATOM | 501 | O | THR | A | 79 | 92.491 | 78.934 | 19.575 | 1.00 | 19.32 | O |
| ATOM | 502 | N | ARG | A | 80 | 91.828 | 80.994 | 18.973 | 1.00 | 17.07 | N |
| ATOM | 503 | CA | ARG | A | 80 | 92.684 | 81.692 | 19.917 | 1.00 | 15.38 | C |
| ATOM | 504 | CB | ARG | A | 80 | 92.779 | 83.154 | 19.562 | 1.00 | 15.28 | C |
| ATOM | 505 | CG | ARG | A | 80 | 93.606 | 83.447 | 18.330 | 1.00 | 15.27 | C |
| ATOM | 506 | CD | ARG | A | 80 | 93.749 | 84.908 | 18.089 | 1.00 | 14.10 | C |
| ATOM | 507 | NE | ARG | A | 80 | 94.368 | 85.211 | 16.793 | 1.00 | 15.86 | N |
| ATOM | 508 | CZ | ARG | A | 80 | 94.653 | 86.445 | 16.403 | 1.00 | 18.63 | C |
| ATOM | 509 | NH1 | ARG | A | 80 | 94.420 | 87.471 | 17.235 | 1.00 | 19.97 | N |
| ATOM | 510 | NH2 | ARG | A | 80 | 95.175 | 86.667 | 15.200 | 1.00 | 17.50 | N |
| ATOM | 511 | C | ARG | A | 80 | 92.216 | 81.591 | 21.348 | 1.00 | 14.85 | C |
| ATOM | 512 | O | ARG | A | 80 | 91.016 | 81.649 | 21.631 | 1.00 | 15.03 | O |
| ATOM | 513 | N | GLN | A | 81 | 93.176 | 81.434 | 22.250 | 1.00 | 13.43 | N |
| ATOM | 514 | CA | GLN | A | 81 | 92.960 | 81.551 | 23.677 | 1.00 | 12.84 | C |
| ATOM | 515 | CB | GLN | A | 81 | 93.216 | 80.218 | 24.373 | 1.00 | 12.99 | C |
| ATOM | 516 | CG | GLN | A | 81 | 92.335 | 79.088 | 23.875 | 1.00 | 12.25 | C |
| ATOM | 517 | CD | GLN | A | 81 | 92.683 | 77.803 | 24.553 | 1.00 | 12.80 | C |
| ATOM | 518 | OE1 | GLN | A | 81 | 92.991 | 77.815 | 25.757 | 1.00 | 14.57 | O |
| ATOM | 519 | NE2 | GLN | A | 81 | 92.694 | 76.693 | 23.801 | 1.00 | 12.80 | N |
| ATOM | 520 | C | GLN | A | 81 | 93.954 | 82.614 | 24.154 | 1.00 | 12.26 | C |
| ATOM | 521 | O | GLN | A | 81 | 95.127 | 82.601 | 23.758 | 1.00 | 10.85 | O |
| ATOM | 522 | N | VAL | A | 82 | 93.449 | 83.560 | 24.941 | 1.00 | 10.90 | N |
| ATOM | 523 | CA | VAL | A | 82 | 94.248 | 84.694 | 25.378 | 1.00 | 11.96 | C |
| ATOM | 524 | CB | VAL | A | 82 | 93.604 | 86.012 | 24.927 | 1.00 | 11.17 | C |
| ATOM | 525 | CG1 | VAL | A | 82 | 94.484 | 87.219 | 25.342 | 1.00 | 14.51 | C |
| ATOM | 526 | CG2 | VAL | A | 82 | 93.337 | 85.961 | 23.408 | 1.00 | 12.75 | C |
| ATOM | 527 | C | VAL | A | 82 | 94.394 | 84.699 | 26.877 | 1.00 | 11.88 | C |
| ATOM | 528 | O | VAL | A | 82 | 93.389 | 84.626 | 27.614 | 1.00 | 12.66 | O |
| ATOM | 529 | N | PHE | A | 83 | 95.647 | 84.786 | 27.331 | 1.00 | 11.21 | N |
| ATOM | 530 | CA | PHE | A | 83 | 95.982 | 84.895 | 28.746 | 1.00 | 11.06 | C |
| ATOM | 531 | CB | PHE | A | 83 | 96.731 | 83.644 | 29.196 | 1.00 | 9.80 | C |
| ATOM | 532 | CG | PHE | A | 83 | 95.903 | 82.431 | 29.129 | 1.00 | 10.10 | C |
| ATOM | 533 | CD1 | PHE | A | 83 | 95.818 | 81.701 | 27.944 | 1.00 | 12.67 | C |
| ATOM | 534 | CE1 | PHE | A | 83 | 95.004 | 80.581 | 27.859 | 1.00 | 12.70 | C |
| ATOM | 535 | CZ | PHE | A | 83 | 94.265 | 80.222 | 28.957 | 1.00 | 10.55 | C |
| ATOM | 536 | CE2 | PHE | A | 83 | 94.323 | 80.969 | 30.116 | 1.00 | 10.59 | C |
| ATOM | 537 | CD2 | PHE | A | 83 | 95.128 | 82.066 | 30.197 | 1.00 | 12.37 | C |
| ATOM | 538 | C | PHE | A | 83 | 96.793 | 86.138 | 29.038 | 1.00 | 10.97 | C |
| ATOM | 539 | O | PHE | A | 83 | 97.222 | 86.849 | 28.139 | 1.00 | 12.12 | O |
| ATOM | 540 | N | ALA | A | 84 | 96.940 | 86.408 | 30.329 | 1.00 | 11.92 | N |
| ATOM | 541 | CA | ALA | A | 84 | 97.809 | 87.457 | 30.857 | 1.00 | 11.76 | C |
| ATOM | 542 | CB | ALA | A | 84 | 96.988 | 88.412 | 31.737 | 1.00 | 12.77 | C |
| ATOM | 543 | C | ALA | A | 84 | 98.940 | 86.809 | 31.666 | 1.00 | 11.67 | C |
| ATOM | 544 | O | ALA | A | 84 | 98.888 | 85.602 | 32.028 | 1.00 | 10.96 | O |
| ATOM | 545 | N | VAL | A | 85 | 99.999 | 87.595 | 31.903 | 1.00 | 11.25 | N |
| ATOM | 546 | CA | VAL | A | 85 | 101.124 | 87.169 | 32.704 | 1.00 | 12.38 | C |
| ATOM | 547 | CB | VAL | A | 85 | 102.400 | 88.000 | 32.328 | 1.00 | 12.77 | C |
| ATOM | 548 | CG1 | VAL | A | 85 | 103.580 | 87.616 | 33.224 | 1.00 | 13.25 | C |
| ATOM | 549 | CG2 | VAL | A | 85 | 102.730 | 87.774 | 30.868 | 1.00 | 13.25 | C |
| ATOM | 550 | C | VAL | A | 85 | 100.874 | 87.259 | 34.226 | 1.00 | 12.98 | C |
| ATOM | 551 | O | VAL | A | 85 | 100.522 | 88.331 | 34.787 | 1.00 | 13.60 | O |
| ATOM | 552 | N | GLN | A | 86 | 101.050 | 86.128 | 34.890 | 1.00 | 12.11 | N |
| ATOM | 553 | CA | GLN | A | 86 | 100.918 | 86.032 | 36.334 | 1.00 | 12.49 | C |
| ATOM | 554 | CB | GLN | A | 86 | 100.216 | 84.756 | 36.737 | 1.00 | 12.39 | C |
| ATOM | 555 | CG | GLN | A | 86 | 99.626 | 84.798 | 38.182 | 1.00 | 17.07 | C |
| ATOM | 556 | CD | GLN | A | 86 | 98.484 | 85.850 | 38.371 | 1.00 | 22.19 | C |
| ATOM | 557 | OE1 | GLN | A | 86 | 98.529 | 86.673 | 39.301 | 1.00 | 26.29 | O |
| ATOM | 558 | NE2 | GLN | A | 86 | 97.479 | 85.801 | 37.525 | 1.00 | 22.42 | N |
| ATOM | 559 | C | GLN | A | 86 | 102.244 | 86.122 | 37.085 | 1.00 | 11.40 | C |
| ATOM | 560 | O | GLN | A | 86 | 102.307 | 86.822 | 38.097 | 1.00 | 12.75 | O |
| ATOM | 561 | N | ARG | A | 87 | 103.271 | 85.438 | 36.589 | 1.00 | 11.15 | N |
| ATOM | 562 | CA | ARG | A | 87 | 104.598 | 85.356 | 37.231 | 1.00 | 10.91 | C |
| ATOM | 563 | CB | ARG | A | 87 | 104.725 | 84.110 | 38.090 | 1.00 | 11.05 | C |
| ATOM | 564 | CG | ARG | A | 87 | 103.684 | 84.009 | 39.211 | 1.00 | 14.74 | C |
| ATOM | 565 | CD | ARG | A | 87 | 104.066 | 82.980 | 40.220 | 1.00 | 18.34 | C |
| ATOM | 566 | NE | ARG | A | 87 | 103.144 | 82.976 | 41.342 | 1.00 | 22.17 | N |
| ATOM | 567 | CZ | ARG | A | 87 | 103.415 | 82.374 | 42.486 | 1.00 | 29.72 | C |
| ATOM | 568 | NH1 | ARG | A | 87 | 104.528 | 81.658 | 42.620 | 1.00 | 33.93 | N |
| ATOM | 569 | NH2 | ARG | A | 87 | 102.555 | 82.439 | 43.494 | 1.00 | 32.37 | N |
| ATOM | 570 | C | ARG | A | 87 | 105.725 | 85.332 | 36.205 | 1.00 | 9.91 | C |
| ATOM | 571 | O | ARG | A | 87 | 105.549 | 84.842 | 35.055 | 1.00 | 7.94 | O |
| ATOM | 572 | N | ILE | A | 88 | 106.873 | 85.858 | 36.623 | 1.00 | 9.45 | N |
| ATOM | 573 | CA | ILE | A | 88 | 108.018 | 86.050 | 35.726 | 1.00 | 8.84 | C |
| ATOM | 574 | CB | ILE | A | 88 | 108.277 | 87.585 | 35.497 | 1.00 | 9.73 | C |
| ATOM | 575 | CG1 | ILE | A | 88 | 107.097 | 88.289 | 34.830 | 1.00 | 11.64 | C |
| ATOM | 576 | CD1 | ILE | A | 88 | 107.067 | 89.748 | 35.060 | 1.00 | 15.34 | C |
| ATOM | 577 | CG2 | ILE | A | 88 | 109.598 | 87.832 | 34.725 | 1.00 | 10.90 | C |
| ATOM | 578 | C | ILE | A | 88 | 109.235 | 85.384 | 36.381 | 1.00 | 8.47 | C |
| ATOM | 579 | O | ILE | A | 88 | 109.531 | 85.599 | 37.584 | 1.00 | 9.58 | O |
| ATOM | 580 | N | PHE | A | 89 | 109.953 | 84.586 | 35.605 | 1.00 | 8.18 | N |
| ATOM | 581 | CA | PHE | A | 89 | 111.196 | 83.965 | 36.048 | 1.00 | 9.56 | C |
| ATOM | 582 | CB | PHE | A | 89 | 111.028 | 82.454 | 36.116 | 1.00 | 9.07 | C |
| ATOM | 583 | CG | PHE | A | 89 | 109.875 | 81.992 | 36.956 | 1.00 | 9.33 | C |
| ATOM | 584 | CD1 | PHE | A | 89 | 110.095 | 81.501 | 38.228 | 1.00 | 13.88 | C |
| ATOM | 585 | CE1 | PHE | A | 89 | 109.006 | 81.063 | 39.029 | 1.00 | 12.65 | C |
| ATOM | 586 | CZ | PHE | A | 89 | 107.760 | 81.125 | 38.543 | 1.00 | 11.19 | C |
| ATOM | 587 | CE2 | PHE | A | 89 | 107.506 | 81.594 | 37.248 | 1.00 | 12.15 | C |
| ATOM | 588 | CD2 | PHE | A | 89 | 108.557 | 82.027 | 36.457 | 1.00 | 9.92 | C |
| ATOM | 589 | C | PHE | A | 89 | 112.328 | 84.285 | 35.025 | 1.00 | 9.24 | C |
| ATOM | 590 | O | PHE | A | 89 | 112.186 | 84.001 | 33.834 | 1.00 | 9.43 | O |
| ATOM | 591 | N | GLU | A | 90 | 113.456 | 84.780 | 35.520 | 1.00 | 9.95 | N |
| ATOM | 592 | CA | GLU | A | 90 | 114.670 | 84.973 | 34.744 | 1.00 | 10.90 | C |
| ATOM | 593 | CB | GLU | A | 90 | 114.844 | 86.458 | 34.382 | 1.00 | 11.12 | C |
| ATOM | 594 | CG | GLU | A | 90 | 113.742 | 86.940 | 33.455 | 1.00 | 12.04 | C |
| ATOM | 595 | CD | GLU | A | 90 | 113.873 | 88.383 | 33.004 | 1.00 | 15.76 | C |
| ATOM | 596 | OE1 | GLU | A | 90 | 114.676 | 89.093 | 33.576 | 1.00 | 18.23 | O |
| ATOM | 597 | OE2 | GLU | A | 90 | 113.100 | 88.821 | 32.110 | 1.00 | 19.65 | O |
| ATOM | 598 | C | GLU | A | 90 | 115.881 | 84.502 | 35.516 | 1.00 | 11.29 | C |
| ATOM | 599 | O | GLU | A | 90 | 115.892 | 84.550 | 36.750 | 1.00 | 10.74 | O |
| ATOM | 600 | N | ASP | A | 91 | 116.881 | 83.993 | 34.804 | 1.00 | 12.73 | N |
| ATOM | 601 | CA | ASP | A | 91 | 118.072 | 83.450 | 35.471 | 1.00 | 13.35 | C |
| ATOM | 602 | CB | ASP | A | 91 | 118.057 | 81.925 | 35.499 | 1.00 | 15.11 | C |
| ATOM | 603 | CG | ASP | A | 91 | 119.107 | 81.345 | 36.476 | 1.00 | 16.74 | C |
| ATOM | 604 | OD1 | ASP | A | 91 | 119.009 | 81.592 | 37.681 | 1.00 | 21.63 | O |
| ATOM | 605 | OD2 | ASP | A | 91 | 120.054 | 80.632 | 36.128 | 1.00 | 25.34 | O |
| ATOM | 606 | C | ASP | A | 91 | 119.346 | 83.946 | 34.847 | 1.00 | 13.81 | C |
| ATOM | 607 | O | ASP | A | 91 | 119.967 | 83.260 | 34.015 | 1.00 | 13.97 | O |
| ATOM | 608 | N | GLY | A | 92 | 119.730 | 85.131 | 35.296 | 1.00 | 14.16 | N |
| ATOM | 609 | CA | GLY | A | 92 | 120.992 | 85.754 | 34.927 | 1.00 | 14.67 | C |
| ATOM | 610 | C | GLY | A | 92 | 120.967 | 86.428 | 33.583 | 1.00 | 13.99 | C |
| ATOM | 611 | O | GLY | A | 92 | 122.000 | 86.567 | 32.939 | 1.00 | 16.71 | O |
| ATOM | 612 | N | TYR | A | 94 | 119.812 | 86.948 | 33.188 | 1.00 | 13.20 | N |
| ATOM | 613 | CA | TYR | A | 94 | 119.669 | 87.674 | 31.931 | 1.00 | 12.65 | C |
| ATOM | 614 | CB | TYR | A | 94 | 118.219 | 88.119 | 31.694 | 1.00 | 11.77 | C |
| ATOM | 615 | CG | TYR | A | 94 | 118.036 | 89.117 | 30.563 | 1.00 | 11.19 | C |
| ATOM | 616 | CD1 | TYR | A | 94 | 117.313 | 90.286 | 30.743 | 1.00 | 10.13 | C |
| ATOM | 617 | CE1 | TYR | A | 94 | 117.159 | 91.184 | 29.699 | 1.00 | 14.67 | C |
| ATOM | 618 | CZ | TYR | A | 94 | 117.687 | 90.876 | 28.451 | 1.00 | 13.34 | C |
| ATOM | 619 | OH | TYR | A | 94 | 117.531 | 91.724 | 27.387 | 1.00 | 17.30 | O |
| ATOM | 620 | CE2 | TYR | A | 94 | 118.359 | 89.708 | 28.254 | 1.00 | 13.64 | C |
| ATOM | 621 | CD2 | TYR | A | 94 | 118.532 | 88.846 | 29.307 | 1.00 | 12.31 | C |
| ATOM | 622 | C | TYR | A | 94 | 120.573 | 88.892 | 31.920 | 1.00 | 11.84 | C |
| ATOM | 623 | O | TYR | A | 94 | 120.462 | 89.725 | 32.759 | 1.00 | 13.14 | O |
| ATOM | 624 | N | ASP | A | 95 | 121.455 | 88.969 | 30.936 | 1.00 | 13.29 | N |
| ATOM | 625 | CA | ASP | A | 95 | 122.391 | 90.067 | 30.749 | 1.00 | 13.23 | C |
| ATOM | 626 | CB | ASP | A | 95 | 123.781 | 89.486 | 30.470 | 1.00 | 13.73 | C |
| ATOM | 627 | CG | ASP | A | 95 | 124.842 | 90.534 | 30.226 | 1.00 | 14.41 | C |
| ATOM | 628 | OD1 | ASP | A | 95 | 124.523 | 91.645 | 29.764 | 1.00 | 15.54 | O |
| ATOM | 629 | OD2 | ASP | A | 95 | 126.053 | 90.293 | 30.486 | 1.00 | 15.05 | O |
| ATOM | 630 | C | ASP | A | 95 | 121.899 | 90.890 | 29.567 | 1.00 | 13.77 | C |
| ATOM | 631 | O | ASP | A | 95 | 122.064 | 90.487 | 28.434 | 1.00 | 12.43 | O |
| ATOM | 632 | N | PRO | A | 98 | 121.324 | 92.048 | 29.824 | 1.00 | 14.77 | N |
| ATOM | 633 | CA | PRO | A | 98 | 120.713 | 92.853 | 28.761 | 1.00 | 17.44 | C |
| ATOM | 634 | CB | PRO | A | 98 | 119.898 | 93.867 | 29.548 | 1.00 | 17.06 | C |
| ATOM | 635 | CG | PRO | A | 98 | 120.659 | 94.033 | 30.791 | 1.00 | 16.73 | C |
| ATOM | 636 | CD | PRO | A | 98 | 121.198 | 92.704 | 31.127 | 1.00 | 15.69 | C |
| ATOM | 637 | C | PRO | A | 98 | 121.693 | 93.566 | 27.839 | 1.00 | 18.74 | C |
| ATOM | 638 | O | PRO | A | 98 | 121.277 | 94.073 | 26.806 | 1.00 | 20.81 | O |
| ATOM | 639 | N | VAL | A | 99 | 122.974 | 93.623 | 28.191 | 1.00 | 20.16 | N |
| ATOM | 640 | CA | VAL | A | 99 | 123.977 | 94.282 | 27.331 | 1.00 | 20.79 | C |
| ATOM | 641 | CB | VAL | A | 99 | 125.060 | 94.943 | 28.202 | 1.00 | 21.37 | C |
| ATOM | 642 | CG1 | VAL | A | 99 | 126.235 | 95.490 | 27.325 | 1.00 | 22.93 | C |
| ATOM | 643 | CG2 | VAL | A | 99 | 124.439 | 96.038 | 28.998 | 1.00 | 23.02 | C |
| ATOM | 644 | C | VAL | A | 99 | 124.606 | 93.315 | 26.365 | 1.00 | 20.37 | C |
| ATOM | 645 | O | VAL | A | 99 | 124.752 | 93.628 | 25.165 | 1.00 | 20.33 | O |
| ATOM | 646 | N | ASN | A | 99A | 124.958 | 92.127 | 26.859 | 1.00 | 18.76 | N |
| ATOM | 647 | CA | ASN | A | 99A | 125.526 | 91.087 | 26.016 | 1.00 | 18.90 | C |
| ATOM | 648 | CB | ASN | A | 99A | 126.632 | 90.353 | 26.752 | 1.00 | 19.50 | C |
| ATOM | 649 | CG | ASN | A | 99A | 127.721 | 91.268 | 27.168 | 1.00 | 20.19 | C |
| ATOM | 650 | OD1 | ASN | A | 99A | 128.360 | 91.903 | 26.310 | 1.00 | 19.67 | O |
| ATOM | 651 | ND2 | ASN | A | 99A | 127.911 | 91.411 | 28.491 | 1.00 | 21.75 | N |
| ATOM | 652 | C | ASN | A | 99A | 124.501 | 90.077 | 25.506 | 1.00 | 18.30 | C |
| ATOM | 653 | O | ASN | A | 99A | 124.871 | 89.123 | 24.832 | 1.00 | 18.26 | O |
| ATOM | 654 | N | LEU | A | 99B | 123.222 | 90.299 | 25.810 | 1.00 | 17.33 | N |
| ATOM | 655 | CA | LEU | A | 99B | 122.172 | 89.285 | 25.559 | 1.00 | 16.80 | C |
| ATOM | 656 | CB | LEU | A | 99B | 121.721 | 89.288 | 24.083 | 1.00 | 16.36 | C |
| ATOM | 657 | CG | LEU | A | 99B | 120.505 | 90.117 | 23.668 | 1.00 | 20.98 | C |
| ATOM | 658 | CD1 | LEU | A | 99B | 120.161 | 91.215 | 24.631 | 1.00 | 22.04 | C |
| ATOM | 659 | CD2 | LEU | A | 99B | 120.648 | 90.664 | 22.231 | 1.00 | 19.87 | C |
| ATOM | 660 | C | LEU | A | 99B | 122.642 | 87.900 | 25.920 | 1.00 | 15.70 | C |
| ATOM | 661 | O | LEU | A | 99B | 122.695 | 87.005 | 25.055 | 1.00 | 16.68 | O |
| ATOM | 662 | N | LEU | A | 100 | 123.010 | 87.711 | 27.189 | 1.00 | 14.90 | N |
| ATOM | 663 | CA | LEU | A | 100 | 123.266 | 86.377 | 27.712 | 1.00 | 14.66 | C |
| ATOM | 664 | CB | LEU | A | 100 | 124.520 | 86.373 | 28.595 | 1.00 | 15.83 | C |
| ATOM | 665 | CG | LEU | A | 100 | 125.811 | 86.787 | 27.856 | 1.00 | 17.61 | C |
| ATOM | 666 | CD1 | LEU | A | 100 | 126.928 | 87.079 | 28.852 | 1.00 | 20.54 | C |
| ATOM | 667 | CD2 | LEU | A | 100 | 126.223 | 85.688 | 26.870 | 1.00 | 18.76 | C |
| ATOM | 668 | C | LEU | A | 100 | 122.053 | 85.920 | 28.528 | 1.00 | 12.64 | C |
| ATOM | 669 | O | LEU | A | 100 | 121.400 | 86.753 | 29.158 | 1.00 | 13.58 | O |
| ATOM | 670 | N | ASN | A | 101 | 121.740 | 84.625 | 28.449 | 1.00 | 10.91 | N |
| ATOM | 671 | CA | ASN | A | 101 | 120.649 | 84.028 | 29.209 | 1.00 | 11.47 | C |
| ATOM | 672 | CB | ASN | A | 101 | 120.963 | 84.034 | 30.711 | 1.00 | 10.51 | C |
| ATOM | 673 | CG | ASN | A | 101 | 122.312 | 83.398 | 31.030 | 1.00 | 14.21 | C |
| ATOM | 674 | OD1 | ASN | A | 101 | 123.112 | 83.972 | 31.780 | 1.00 | 24.34 | O |
| ATOM | 675 | ND2 | ASN | A | 101 | 122.568 | 82.260 | 30.467 | 1.00 | 11.56 | N |
| ATOM | 676 | C | ASN | A | 101 | 119.377 | 84.824 | 28.867 | 1.00 | 10.40 | C |
| ATOM | 677 | O | ASN | A | 101 | 118.642 | 85.280 | 29.728 | 1.00 | 11.58 | O |
| ATOM | 678 | N | ASP | A | 102 | 119.148 | 84.986 | 27.579 | 1.00 | 10.61 | N |
| ATOM | 679 | CA | ASP | A | 102 | 117.997 | 85.744 | 27.075 | 1.00 | 10.62 | C |
| ATOM | 680 | CB | ASP | A | 102 | 118.367 | 86.363 | 25.699 | 1.00 | 10.22 | C |
| ATOM | 681 | CG | ASP | A | 102 | 117.350 | 87.360 | 25.197 | 1.00 | 10.80 | C |
| ATOM | 682 | OD1 | ASP | A | 102 | 116.499 | 87.831 | 26.000 | 1.00 | 8.19 | O |
| ATOM | 683 | OD2 | ASP | A | 102 | 117.353 | 87.739 | 24.009 | 1.00 | 9.31 | O |
| ATOM | 684 | C | ASP | A | 102 | 116.780 | 84.805 | 26.973 | 1.00 | 10.57 | C |
| ATOM | 685 | O | ASP | A | 102 | 116.347 | 84.450 | 25.898 | 1.00 | 11.10 | O |
| ATOM | 686 | N | ILE | A | 103 | 116.284 | 84.411 | 28.137 | 1.00 | 10.58 | N |
| ATOM | 687 | CA | ILE | A | 103 | 115.159 | 83.531 | 28.325 | 1.00 | 10.00 | C |
| ATOM | 688 | CB | ILE | A | 103 | 115.616 | 82.059 | 28.509 | 1.00 | 9.90 | C |
| ATOM | 689 | CG1 | ILE | A | 103 | 114.430 | 81.113 | 28.669 | 1.00 | 12.59 | C |
| ATOM | 690 | CD1 | ILE | A | 103 | 114.866 | 79.616 | 28.586 | 1.00 | 13.11 | C |
| ATOM | 691 | CG2 | ILE | A | 103 | 116.507 | 81.884 | 29.683 | 1.00 | 10.09 | C |
| ATOM | 692 | C | ILE | A | 103 | 114.345 | 84.041 | 29.527 | 1.00 | 10.08 | C |
| ATOM | 693 | O | ILE | A | 103 | 114.885 | 84.446 | 30.574 | 1.00 | 11.06 | O |
| ATOM | 694 | N | VAL | A | 104 | 113.042 | 84.032 | 29.364 | 1.00 | 9.51 | N |
| ATOM | 695 | CA | VAL | A | 104 | 112.101 | 84.267 | 30.463 | 1.00 | 9.13 | C |
| ATOM | 696 | CB | VAL | A | 104 | 111.462 | 85.673 | 30.370 | 1.00 | 9.35 | C |
| ATOM | 697 | CG1 | VAL | A | 104 | 110.681 | 85.904 | 29.038 | 1.00 | 9.78 | C |
| ATOM | 698 | CG2 | VAL | A | 104 | 110.564 | 85.943 | 31.594 | 1.00 | 10.76 | C |
| ATOM | 699 | C | VAL | A | 104 | 111.033 | 83.182 | 30.475 | 1.00 | 9.33 | C |
| ATOM | 700 | O | VAL | A | 104 | 110.610 | 82.730 | 29.413 | 1.00 | 8.73 | O |
| ATOM | 701 | N | ILE | A | 105 | 110.602 | 82.759 | 31.665 | 1.00 | 10.28 | N |
| ATOM | 702 | CA | ILE | A | 105 | 109.427 | 81.884 | 31.798 | 1.00 | 10.04 | C |
| ATOM | 703 | CB | ILE | A | 105 | 109.662 | 80.699 | 32.749 | 1.00 | 10.65 | C |
| ATOM | 704 | CG1 | ILE | A | 105 | 110.898 | 79.877 | 32.363 | 1.00 | 14.36 | C |
| ATOM | 705 | CD1 | ILE | A | 105 | 111.071 | 79.605 | 30.899 | 1.00 | 18.72 | C |
| ATOM | 706 | CG2 | ILE | A | 105 | 108.415 | 79.802 | 32.827 | 1.00 | 12.35 | C |
| ATOM | 707 | C | ILE | A | 105 | 108.336 | 82.758 | 32.348 | 1.00 | 9.31 | C |
| ATOM | 708 | O | ILE | A | 105 | 108.550 | 83.467 | 33.315 | 1.00 | 9.45 | O |
| ATOM | 709 | N | LEU | A | 106 | 107.165 | 82.705 | 31.719 | 1.00 | 9.73 | N |
| ATOM | 710 | CA | LEU | A | 106 | 106.030 | 83.517 | 32.101 | 1.00 | 9.42 | C |
| ATOM | 711 | CB | LEU | A | 106 | 105.592 | 84.420 | 30.948 | 1.00 | 8.94 | C |
| ATOM | 712 | CG | LEU | A | 106 | 106.601 | 85.381 | 30.336 | 1.00 | 9.67 | C |
| ATOM | 713 | CD1 | LEU | A | 106 | 106.078 | 85.956 | 28.962 | 1.00 | 13.43 | C |
| ATOM | 714 | CD2 | LEU | A | 106 | 106.908 | 86.464 | 31.306 | 1.00 | 11.28 | C |
| ATOM | 715 | C | LEU | A | 106 | 104.906 | 82.555 | 32.436 | 1.00 | 9.07 | C |
| ATOM | 716 | O | LEU | A | 106 | 104.463 | 81.799 | 31.581 | 1.00 | 9.22 | O |
| ATOM | 717 | N | GLN | A | 107 | 104.522 | 82.531 | 33.697 | 1.00 | 8.93 | N |
| ATOM | 718 | CA | GLN | A | 107 | 103.326 | 81.824 | 34.133 | 1.00 | 9.56 | C |
| ATOM | 719 | CB | GLN | A | 107 | 103.347 | 81.658 | 35.647 | 1.00 | 10.81 | C |
| ATOM | 720 | CG | GLN | A | 107 | 102.195 | 80.816 | 36.189 | 1.00 | 8.86 | C |
| ATOM | 721 | CD | GLN | A | 107 | 102.355 | 80.459 | 37.667 | 1.00 | 12.52 | C |
| ATOM | 722 | OE1 | GLN | A | 107 | 101.380 | 80.190 | 38.338 | 1.00 | 19.13 | O |
| ATOM | 723 | NE2 | GLN | A | 107 | 103.568 | 80.515 | 38.179 | 1.00 | 10.80 | N |
| ATOM | 724 | C | GLN | A | 107 | 102.143 | 82.654 | 33.690 | 1.00 | 10.12 | C |
| ATOM | 725 | O | GLN | A | 107 | 102.158 | 83.886 | 33.754 | 1.00 | 9.44 | O |
| ATOM | 726 | N | LEU | A | 108 | 101.103 | 81.971 | 33.221 | 1.00 | 9.90 | N |
| ATOM | 727 | CA | LEU | A | 108 | 99.904 | 82.597 | 32.739 | 1.00 | 9.87 | C |
| ATOM | 728 | CB | LEU | A | 108 | 99.312 | 81.712 | 31.658 | 1.00 | 9.36 | C |
| ATOM | 729 | CG | LEU | A | 108 | 99.828 | 81.832 | 30.224 | 1.00 | 14.79 | C |
| ATOM | 730 | CD1 | LEU | A | 108 | 99.730 | 80.567 | 29.463 | 1.00 | 15.95 | C |
| ATOM | 731 | CD2 | LEU | A | 108 | 101.098 | 82.586 | 29.971 | 1.00 | 11.26 | C |
| ATOM | 732 | C | LEU | A | 108 | 98.877 | 82.697 | 33.887 | 1.00 | 10.83 | C |
| ATOM | 733 | O | LEU | A | 108 | 98.957 | 81.975 | 34.896 | 1.00 | 10.11 | O |
| ATOM | 734 | N | ASN | A | 109 | 97.865 | 83.513 | 33.679 | 1.00 | 10.80 | N |
| ATOM | 735 | CA | ASN | A | 109 | 96.839 | 83.734 | 34.719 | 1.00 | 12.10 | C |
| ATOM | 736 | CB | ASN | A | 109 | 96.174 | 85.121 | 34.571 | 1.00 | 11.81 | C |
| ATOM | 737 | CG | ASN | A | 109 | 95.279 | 85.220 | 33.354 | 1.00 | 13.63 | C |
| ATOM | 738 | OD1 | ASN | A | 109 | 95.630 | 84.751 | 32.303 | 1.00 | 9.76 | O |
| ATOM | 739 | ND2 | ASN | A | 109 | 94.137 | 85.890 | 33.494 | 1.00 | 19.82 | N |
| ATOM | 740 | C | ASN | A | 109 | 95.780 | 82.625 | 34.735 | 1.00 | 12.77 | C |
| ATOM | 741 | O | ASN | A | 109 | 94.816 | 82.668 | 35.524 | 1.00 | 16.28 | O |
| ATOM | 742 | N | GLY | A | 110 | 95.984 | 81.601 | 33.932 | 1.00 | 13.77 | N |
| ATOM | 743 | CA | GLY | A | 110 | 95.087 | 80.475 | 33.823 | 1.00 | 14.01 | C |
| ATOM | 744 | C | GLY | A | 110 | 95.766 | 79.370 | 33.019 | 1.00 | 13.87 | C |
| ATOM | 745 | O | GLY | A | 110 | 96.893 | 79.509 | 32.620 | 1.00 | 13.10 | O |
| ATOM | 746 | N | SER | A | 111 | 95.031 | 78.298 | 32.759 | 1.00 | 13.93 | N |
| ATOM | 747 | CA | SER | A | 111 | 95.523 | 77.155 | 31.997 | 1.00 | 13.94 | C |
| ATOM | 748 | CB | SER | A | 111 | 95.288 | 75.897 | 32.783 | 1.00 | 14.28 | C |
| ATOM | 749 | OG | SER | A | 111 | 96.249 | 75.810 | 33.793 | 1.00 | 14.94 | O |
| ATOM | 750 | C | SER | A | 111 | 94.840 | 77.029 | 30.655 | 1.00 | 14.24 | C |
| ATOM | 751 | O | SER | A | 111 | 93.596 | 77.068 | 30.562 | 1.00 | 13.83 | O |
| ATOM | 752 | N | ALA | A | 112 | 95.629 | 76.851 | 29.607 | 1.00 | 12.99 | N |
| ATOM | 753 | CA | ALA | A | 112 | 95.083 | 76.683 | 28.262 | 1.00 | 14.02 | C |
| ATOM | 754 | CB | ALA | A | 112 | 96.217 | 76.618 | 27.278 | 1.00 | 14.53 | C |
| ATOM | 755 | C | ALA | A | 112 | 94.243 | 75.399 | 28.206 | 1.00 | 13.69 | C |
| ATOM | 756 | O | ALA | A | 112 | 94.466 | 74.518 | 28.980 | 1.00 | 13.79 | O |
| ATOM | 757 | N | THR | A | 113 | 93.240 | 75.358 | 27.342 | 1.00 | 14.64 | N |
| ATOM | 758 | CA | THR | A | 113 | 92.558 | 74.140 | 26.961 | 1.00 | 14.99 | C |
| ATOM | 759 | CB | THR | A | 113 | 91.156 | 74.475 | 26.482 | 1.00 | 15.66 | C |
| ATOM | 760 | OG1 | THR | A | 113 | 90.435 | 75.052 | 27.581 | 1.00 | 15.69 | O |
| ATOM | 761 | CG2 | THR | A | 113 | 90.379 | 73.217 | 26.042 | 1.00 | 17.43 | C |
| ATOM | 762 | C | THR | A | 113 | 93.377 | 73.529 | 25.858 | 1.00 | 14.89 | C |
| ATOM | 763 | O | THR | A | 113 | 93.581 | 74.142 | 24.840 | 1.00 | 16.17 | O |
| ATOM | 764 | N | ILE | A | 114 | 93.888 | 72.332 | 26.088 | 1.00 | 14.48 | N |
| ATOM | 765 | CA | ILE | A | 114 | 94.767 | 71.694 | 25.149 | 1.00 | 14.69 | C |
| ATOM | 766 | CB | ILE | A | 114 | 95.654 | 70.661 | 25.886 | 1.00 | 15.06 | C |
| ATOM | 767 | CG1 | ILE | A | 114 | 96.463 | 71.349 | 26.977 | 1.00 | 14.63 | C |
| ATOM | 768 | CD1 | ILE | A | 114 | 97.544 | 72.315 | 26.432 | 1.00 | 14.67 | C |
| ATOM | 769 | CG2 | ILE | A | 114 | 96.574 | 69.941 | 24.909 | 1.00 | 14.98 | C |
| ATOM | 770 | C | ILE | A | 114 | 93.901 | 71.023 | 24.100 | 1.00 | 15.50 | C |
| ATOM | 771 | O | ILE | A | 114 | 92.972 | 70.280 | 24.430 | 1.00 | 14.08 | O |
| ATOM | 772 | N | ASN | A | 115 | 94.195 | 71.274 | 22.837 | 1.00 | 14.55 | N |
| ATOM | 773 | CA | ASN | A | 115 | 93.388 | 70.765 | 21.750 | 1.00 | 15.23 | C |
| ATOM | 774 | CB | ASN | A | 115 | 92.088 | 71.579 | 21.640 | 1.00 | 14.95 | C |
| ATOM | 775 | CG | ASN | A | 115 | 92.321 | 73.095 | 21.407 | 1.00 | 15.09 | C |
| ATOM | 776 | OD1 | ASN | A | 115 | 91.551 | 73.939 | 21.884 | 1.00 | 18.59 | O |
| ATOM | 777 | ND2 | ASN | A | 115 | 93.348 | 73.424 | 20.651 | 1.00 | 12.36 | N |
| ATOM | 778 | C | ASN | A | 115 | 94.217 | 70.778 | 20.466 | 1.00 | 16.18 | C |
| ATOM | 779 | O | ASN | A | 115 | 95.432 | 70.940 | 20.528 | 1.00 | 17.42 | O |
| ATOM | 780 | N | ALA | A | 116 | 93.602 | 70.582 | 19.314 | 1.00 | 17.32 | N |
| ATOM | 781 | CA | ALA | A | 116 | 94.363 | 70.477 | 18.058 | 1.00 | 18.42 | C |
| ATOM | 782 | CB | ALA | A | 116 | 93.399 | 70.340 | 16.882 | 1.00 | 18.85 | C |
| ATOM | 783 | C | ALA | A | 116 | 95.309 | 71.669 | 17.819 | 1.00 | 18.35 | C |
| ATOM | 784 | O | ALA | A | 116 | 96.432 | 71.510 | 17.317 | 1.00 | 20.12 | O |
| ATOM | 785 | N | ASN | A | 117 | 94.832 | 72.846 | 18.186 | 1.00 | 18.50 | N |
| ATOM | 786 | CA | ASN | A | 117 | 95.469 | 74.105 | 17.847 | 1.00 | 17.39 | C |
| ATOM | 787 | CB | ASN | A | 117 | 94.393 | 75.080 | 17.420 | 1.00 | 18.88 | C |
| ATOM | 788 | CG | ASN | A | 117 | 93.616 | 74.586 | 16.213 | 1.00 | 19.68 | C |
| ATOM | 789 | OD1 | ASN | A | 117 | 92.423 | 74.843 | 16.098 | 1.00 | 29.67 | O |
| ATOM | 790 | ND2 | ASN | A | 117 | 94.275 | 73.849 | 15.338 | 1.00 | 16.73 | N |
| ATOM | 791 | C | ASN | A | 117 | 96.302 | 74.718 | 18.961 | 1.00 | 16.13 | C |
| ATOM | 792 | O | ASN | A | 117 | 96.993 | 75.708 | 18.743 | 1.00 | 13.61 | O |
| ATOM | 793 | N | VAL | A | 118 | 96.203 | 74.142 | 20.147 | 1.00 | 14.55 | N |
| ATOM | 794 | CA | VAL | A | 118 | 96.915 | 74.595 | 21.312 | 1.00 | 14.38 | C |
| ATOM | 795 | CB | VAL | A | 118 | 95.977 | 75.401 | 22.193 | 1.00 | 14.42 | C |
| ATOM | 796 | CG1 | VAL | A | 118 | 96.690 | 75.898 | 23.421 | 1.00 | 13.24 | C |
| ATOM | 797 | CG2 | VAL | A | 118 | 95.406 | 76.612 | 21.381 | 1.00 | 15.18 | C |
| ATOM | 798 | C | VAL | A | 118 | 97.522 | 73.387 | 22.043 | 1.00 | 14.41 | C |
| ATOM | 799 | O | VAL | A | 118 | 96.816 | 72.616 | 22.692 | 1.00 | 13.70 | O |
| ATOM | 800 | N | GLN | A | 119 | 98.834 | 73.212 | 21.901 | 1.00 | 13.28 | N |
| ATOM | 801 | CA | GLN | A | 119 | 99.543 | 72.079 | 22.489 | 1.00 | 13.15 | C |
| ATOM | 802 | CB | GLN | A | 119 | 99.834 | 71.021 | 21.425 | 1.00 | 13.35 | C |
| ATOM | 803 | CG | GLN | A | 119 | 98.604 | 70.451 | 20.791 | 1.00 | 13.28 | C |
| ATOM | 804 | CD | GLN | A | 119 | 98.891 | 69.418 | 19.739 | 1.00 | 16.40 | C |
| ATOM | 805 | OE1 | GLN | A | 119 | 98.083 | 69.234 | 18.830 | 1.00 | 20.81 | O |
| ATOM | 806 | NE2 | GLN | A | 119 | 100.000 | 68.716 | 19.871 | 1.00 | 15.50 | N |
| ATOM | 807 | C | GLN | A | 119 | 100.849 | 72.543 | 23.089 | 1.00 | 13.70 | C |
| ATOM | 808 | O | GLN | A | 119 | 101.405 | 73.583 | 22.676 | 1.00 | 13.06 | O |
| ATOM | 809 | N | VAL | A | 120 | 101.331 | 71.780 | 24.072 | 1.00 | 12.50 | N |
| ATOM | 810 | CA | VAL | A | 120 | 102.590 | 72.058 | 24.688 | 1.00 | 13.11 | C |
| ATOM | 811 | CB | VAL | A | 120 | 102.711 | 71.493 | 26.141 | 1.00 | 12.87 | C |
| ATOM | 812 | CG1 | VAL | A | 120 | 101.606 | 72.072 | 27.045 | 1.00 | 13.75 | C |
| ATOM | 813 | CG2 | VAL | A | 120 | 102.692 | 69.996 | 26.138 | 1.00 | 14.57 | C |
| ATOM | 814 | C | VAL | A | 120 | 103.706 | 71.564 | 23.809 | 1.00 | 12.49 | C |
| ATOM | 815 | O | VAL | A | 120 | 103.581 | 70.517 | 23.149 | 1.00 | 13.27 | O |
| ATOM | 816 | N | ALA | A | 121 | 104.810 | 72.296 | 23.806 | 1.00 | 12.75 | N |
| ATOM | 817 | CA | ALA | A | 121 | 106.012 | 71.864 | 23.067 | 1.00 | 13.64 | C |
| ATOM | 818 | CB | ALA | A | 121 | 106.919 | 73.043 | 22.790 | 1.00 | 13.62 | C |
| ATOM | 819 | C | ALA | A | 121 | 106.792 | 70.791 | 23.843 | 1.00 | 14.26 | C |
| ATOM | 820 | O | ALA | A | 121 | 106.686 | 70.680 | 25.076 | 1.00 | 14.66 | O |
| ATOM | 821 | N | GLN | A | 122 | 107.636 | 70.066 | 23.120 | 1.00 | 13.47 | N |
| ATOM | 822 | CA | GLN | A | 122 | 108.505 | 69.096 | 23.724 | 1.00 | 14.00 | C |
| ATOM | 823 | CB | GLN | A | 122 | 108.415 | 67.784 | 22.903 | 1.00 | 14.57 | C |
| ATOM | 824 | CG | GLN | A | 122 | 107.013 | 67.172 | 22.936 | 1.00 | 14.49 | C |
| ATOM | 825 | CD | GLN | A | 122 | 106.701 | 66.692 | 24.335 | 1.00 | 15.84 | C |
| ATOM | 826 | OE1 | GLN | A | 122 | 107.552 | 66.017 | 24.940 | 1.00 | 14.59 | O |
| ATOM | 827 | NE2 | GLN | A | 122 | 105.560 | 67.109 | 24.899 | 1.00 | 13.54 | N |
| ATOM | 828 | C | GLN | A | 122 | 109.928 | 69.659 | 23.812 | 1.00 | 13.75 | C |
| ATOM | 829 | O | GLN | A | 122 | 110.408 | 70.293 | 22.886 | 1.00 | 14.28 | O |
| ATOM | 830 | N | LEU | A | 123 | 110.602 | 69.431 | 24.923 | 1.00 | 13.15 | N |
| ATOM | 831 | CA | LEU | A | 123 | 111.908 | 70.021 | 25.178 | 1.00 | 14.58 | C |
| ATOM | 832 | CB | LEU | A | 123 | 111.878 | 70.800 | 26.477 | 1.00 | 15.08 | C |
| ATOM | 833 | CG | LEU | A | 123 | 110.905 | 71.981 | 26.628 | 1.00 | 19.04 | C |
| ATOM | 834 | CD1 | LEU | A | 123 | 111.350 | 72.896 | 27.745 | 1.00 | 19.82 | C |
| ATOM | 835 | CD2 | LEU | A | 123 | 110.681 | 72.774 | 25.390 | 1.00 | 18.81 | C |
| ATOM | 836 | C | LEU | A | 123 | 113.024 | 68.960 | 25.261 | 1.00 | 13.90 | C |
| ATOM | 837 | O | LEU | A | 123 | 112.748 | 67.810 | 25.523 | 1.00 | 14.07 | O |
| ATOM | 838 | N | PRO | A | 124 | 114.265 | 69.354 | 24.985 | 1.00 | 15.42 | N |
| ATOM | 839 | CA | PRO | A | 124 | 115.437 | 68.450 | 25.097 | 1.00 | 15.42 | C |
| ATOM | 840 | CB | PRO | A | 124 | 116.563 | 69.241 | 24.403 | 1.00 | 15.98 | C |
| ATOM | 841 | CG | PRO | A | 124 | 116.175 | 70.680 | 24.478 | 1.00 | 16.28 | C |
| ATOM | 842 | CD | PRO | A | 124 | 114.647 | 70.705 | 24.519 | 1.00 | 15.41 | C |
| ATOM | 843 | C | PRO | A | 124 | 115.851 | 68.161 | 26.527 | 1.00 | 16.52 | C |
| ATOM | 844 | O | PRO | A | 124 | 115.387 | 68.813 | 27.481 | 1.00 | 15.07 | O |
| ATOM | 845 | N | ALA | A | 125 | 116.754 | 67.184 | 26.691 | 1.00 | 16.92 | N |
| ATOM | 846 | CA | ALA | A | 125 | 117.389 | 66.937 | 27.974 | 1.00 | 16.61 | C |
| ATOM | 847 | CB | ALA | A | 125 | 118.220 | 65.600 | 27.953 | 1.00 | 17.45 | C |
| ATOM | 848 | C | ALA | A | 125 | 118.318 | 68.047 | 28.251 | 1.00 | 16.94 | C |
| ATOM | 849 | O | ALA | A | 125 | 118.879 | 68.604 | 27.325 | 1.00 | 18.92 | O |
| ATOM | 850 | N | GLN | A | 126 | 118.527 | 68.327 | 29.539 | 1.00 | 16.66 | N |
| ATOM | 851 | CA | GLN | A | 126 | 119.519 | 69.260 | 30.019 | 1.00 | 17.37 | C |
| ATOM | 852 | CB | GLN | A | 126 | 119.597 | 69.213 | 31.543 | 1.00 | 17.31 | C |
| ATOM | 853 | CG | GLN | A | 126 | 120.738 | 69.988 | 32.181 | 1.00 | 17.28 | C |
| ATOM | 854 | CD | GLN | A | 126 | 120.520 | 71.479 | 32.137 | 1.00 | 19.73 | C |
| ATOM | 855 | OE1 | GLN | A | 126 | 119.370 | 71.949 | 32.229 | 1.00 | 18.91 | O |
| ATOM | 856 | NE2 | GLN | A | 126 | 121.608 | 72.239 | 32.021 | 1.00 | 14.76 | N |
| ATOM | 857 | C | GLN | A | 126 | 120.881 | 68.940 | 29.418 | 1.00 | 19.68 | C |
| ATOM | 858 | O | GLN | A | 126 | 121.233 | 67.754 | 29.231 | 1.00 | 17.99 | O |
| ATOM | 859 | N | GLY | A | 127 | 121.600 | 70.010 | 29.091 | 1.00 | 20.90 | N |
| ATOM | 860 | CA | GLY | A | 127 | 122.908 | 69.931 | 28.454 | 1.00 | 22.26 | C |
| ATOM | 861 | C | GLY | A | 127 | 123.032 | 69.343 | 27.056 | 1.00 | 23.27 | C |
| ATOM | 862 | O | GLY | A | 127 | 124.155 | 69.227 | 26.585 | 1.00 | 24.36 | O |
| ATOM | 863 | N | ARG | A | 128 | 121.937 | 68.961 | 26.407 | 1.00 | 24.06 | N |
| ATOM | 864 | CA | ARG | A | 128 | 121.960 | 68.348 | 25.086 | 1.00 | 25.35 | C |
| ATOM | 865 | CB | ARG | A | 128 | 120.565 | 67.919 | 24.665 | 1.00 | 25.93 | C |
| ATOM | 866 | CG | ARG | A | 128 | 120.455 | 67.305 | 23.285 | 1.00 | 30.10 | C |
| ATOM | 867 | CD | ARG | A | 128 | 121.001 | 65.888 | 23.204 | 1.00 | 34.74 | C |
| ATOM | 868 | NE | ARG | A | 128 | 122.044 | 65.740 | 22.188 | 1.00 | 40.56 | N |
| ATOM | 869 | CZ | ARG | A | 128 | 121.818 | 65.597 | 20.879 | 1.00 | 42.85 | C |
| ATOM | 870 | NH1 | ARG | A | 128 | 120.581 | 65.598 | 20.406 | 1.00 | 43.56 | N |
| ATOM | 871 | NH2 | ARG | A | 128 | 122.834 | 65.451 | 20.042 | 1.00 | 43.43 | N |
| ATOM | 872 | C | ARG | A | 128 | 122.493 | 69.334 | 24.046 | 1.00 | 26.40 | C |
| ATOM | 873 | O | ARG | A | 128 | 122.016 | 70.465 | 23.950 | 1.00 | 24.41 | O |
| ATOM | 874 | N | ARG | A | 129 | 123.473 | 68.867 | 23.271 | 1.00 | 27.32 | N |
| ATOM | 875 | CA | ARG | A | 129 | 124.264 | 69.727 | 22.390 | 1.00 | 28.94 | C |
| ATOM | 876 | CB | ARG | A | 129 | 125.743 | 69.617 | 22.726 | 1.00 | 29.93 | C |
| ATOM | 877 | CG | ARG | A | 129 | 126.281 | 70.840 | 23.504 | 1.00 | 34.80 | C |
| ATOM | 878 | CD | ARG | A | 129 | 127.790 | 70.782 | 23.810 | 1.00 | 39.84 | C |
| ATOM | 879 | NE | ARG | A | 129 | 128.498 | 71.960 | 23.300 | 1.00 | 44.90 | N |
| ATOM | 880 | CZ | ARG | A | 129 | 128.426 | 73.198 | 23.829 | 1.00 | 49.55 | C |
| ATOM | 881 | NH1 | ARG | A | 129 | 127.669 | 73.452 | 24.904 | 1.00 | 49.62 | N |
| ATOM | 882 | NH2 | ARG | A | 129 | 129.127 | 74.194 | 23.272 | 1.00 | 51.15 | N |
| ATOM | 883 | C | ARG | A | 129 | 124.015 | 69.291 | 20.965 | 1.00 | 28.17 | C |
| ATOM | 884 | O | ARG | A | 129 | 124.106 | 68.098 | 20.652 | 1.00 | 28.27 | O |
| ATOM | 885 | N | LEU | A | 130 | 123.617 | 70.250 | 20.122 | 1.00 | 27.01 | N |
| ATOM | 886 | CA | LEU | A | 130 | 123.476 | 70.009 | 18.708 | 1.00 | 25.65 | C |
| ATOM | 887 | CB | LEU | A | 130 | 122.268 | 70.715 | 18.111 | 1.00 | 26.09 | C |
| ATOM | 888 | CG | LEU | A | 130 | 120.861 | 70.397 | 18.668 | 1.00 | 25.74 | C |
| ATOM | 889 | CD1 | LEU | A | 130 | 119.878 | 71.130 | 17.850 | 1.00 | 26.98 | C |
| ATOM | 890 | CD2 | LEU | A | 130 | 120.540 | 68.929 | 18.582 | 1.00 | 25.00 | C |
| ATOM | 891 | C | LEU | A | 130 | 124.759 | 70.585 | 18.123 | 1.00 | 25.31 | C |
| ATOM | 892 | O | LEU | A | 130 | 125.168 | 71.752 | 18.374 | 1.00 | 23.93 | O |
| ATOM | 893 | N | GLY | A | 131 | 125.392 | 69.743 | 17.340 | 1.00 | 24.97 | N |
| ATOM | 894 | CA | GLY | A | 131 | 126.688 | 70.053 | 16.806 | 1.00 | 24.28 | C |
| ATOM | 895 | C | GLY | A | 131 | 126.471 | 70.852 | 15.570 | 1.00 | 23.60 | C |
| ATOM | 896 | O | GLY | A | 131 | 125.397 | 70.808 | 14.941 | 1.00 | 23.60 | O |
| ATOM | 897 | N | ASN | A | 132 | 127.530 | 71.560 | 15.189 | 1.00 | 22.57 | N |
| ATOM | 898 | CA | ASN | A | 132 | 127.577 | 72.153 | 13.886 | 1.00 | 21.73 | C |
| ATOM | 899 | CB | ASN | A | 132 | 129.015 | 72.296 | 13.373 | 1.00 | 21.46 | C |
| ATOM | 900 | CG | ASN | A | 132 | 129.052 | 73.040 | 12.086 | 1.00 | 19.53 | C |
| ATOM | 901 | OD1 | ASN | A | 132 | 128.691 | 74.177 | 12.060 | 1.00 | 19.45 | O |
| ATOM | 902 | ND2 | ASN | A | 132 | 129.453 | 72.400 | 11.008 | 1.00 | 22.11 | N |
| ATOM | 903 | C | ASN | A | 132 | 126.866 | 71.265 | 12.893 | 1.00 | 19.85 | C |
| ATOM | 904 | O | ASN | A | 132 | 127.079 | 70.092 | 12.890 | 1.00 | 22.23 | O |
| ATOM | 905 | N | GLY | A | 133 | 126.039 | 71.846 | 12.052 | 1.00 | 20.25 | N |
| ATOM | 906 | CA | GLY | A | 133 | 125.455 | 71.146 | 10.912 | 1.00 | 20.54 | C |
| ATOM | 907 | C | GLY | A | 133 | 124.065 | 70.537 | 10.930 | 1.00 | 22.03 | C |
| ATOM | 908 | O | GLY | A | 133 | 123.611 | 70.116 | 9.869 | 1.00 | 22.54 | O |
| ATOM | 909 | N | VAL | A | 134 | 123.404 | 70.456 | 12.094 | 1.00 | 22.90 | N |
| ATOM | 910 | CA | VAL | A | 134 | 122.042 | 69.888 | 12.231 | 1.00 | 23.35 | C |
| ATOM | 911 | CB | VAL | A | 134 | 121.678 | 69.624 | 13.733 | 1.00 | 22.90 | C |
| ATOM | 912 | CG1 | VAL | A | 134 | 120.200 | 69.177 | 13.895 | 1.00 | 25.76 | C |
| ATOM | 913 | CG2 | VAL | A | 134 | 122.606 | 68.565 | 14.338 | 1.00 | 23.93 | C |
| ATOM | 914 | C | VAL | A | 134 | 120.954 | 70.802 | 11.644 | 1.00 | 23.19 | C |
| ATOM | 915 | O | VAL | A | 134 | 121.077 | 72.025 | 11.694 | 1.00 | 21.95 | O |
| ATOM | 916 | N | GLN | A | 135 | 119.896 | 70.185 | 11.146 | 1.00 | 22.33 | N |
| ATOM | 917 | CA | GLN | A | 135 | 118.839 | 70.865 | 10.450 | 1.00 | 23.77 | C |
| ATOM | 918 | CB | GLN | A | 135 | 118.426 | 70.072 | 9.216 | 1.00 | 24.48 | C |
| ATOM | 919 | CG | GLN | A | 135 | 119.455 | 70.173 | 8.057 | 1.00 | 28.58 | C |
| ATOM | 920 | CD | GLN | A | 135 | 119.544 | 71.597 | 7.463 | 1.00 | 34.10 | C |
| ATOM | 921 | OE1 | GLN | A | 135 | 118.576 | 72.094 | 6.854 | 1.00 | 37.78 | O |
| ATOM | 922 | NE2 | GLN | A | 135 | 120.699 | 72.252 | 7.643 | 1.00 | 36.71 | N |
| ATOM | 923 | C | GLN | A | 135 | 117.622 | 71.075 | 11.313 | 1.00 | 22.76 | C |
| ATOM | 924 | O | GLN | A | 135 | 117.096 | 70.124 | 11.930 | 1.00 | 23.42 | O |
| ATOM | 925 | N | CYS | A | 136 | 117.143 | 72.310 | 11.288 | 1.00 | 20.46 | N |
| ATOM | 926 | CA | CYS | A | 136 | 116.047 | 72.751 | 12.142 | 1.00 | 18.68 | C |
| ATOM | 927 | CB | CYS | A | 136 | 116.624 | 73.550 | 13.322 | 1.00 | 17.92 | C |
| ATOM | 928 | SG | CYS | A | 136 | 117.911 | 72.789 | 14.328 | 1.00 | 19.61 | S |
| ATOM | 929 | C | CYS | A | 136 | 115.080 | 73.644 | 11.376 | 1.00 | 17.17 | C |
| ATOM | 930 | O | CYS | A | 136 | 115.235 | 73.919 | 10.187 | 1.00 | 15.95 | O |
| ATOM | 931 | N | LEU | A | 137 | 114.080 | 74.126 | 12.108 | 1.00 | 16.15 | N |
| ATOM | 932 | CA | LEU | A | 137 | 113.029 | 74.968 | 11.582 | 1.00 | 14.51 | C |
| ATOM | 933 | CB | LEU | A | 137 | 111.752 | 74.166 | 11.479 | 1.00 | 14.53 | C |
| ATOM | 934 | CG | LEU | A | 137 | 110.632 | 74.782 | 10.696 | 1.00 | 17.31 | C |
| ATOM | 935 | CD1 | LEU | A | 137 | 110.940 | 74.743 | 9.175 | 1.00 | 18.20 | C |
| ATOM | 936 | CD2 | LEU | A | 137 | 109.343 | 74.027 | 10.948 | 1.00 | 21.48 | C |
| ATOM | 937 | C | LEU | A | 137 | 112.823 | 76.158 | 12.500 | 1.00 | 12.75 | C |
| ATOM | 938 | O | LEU | A | 137 | 112.530 | 75.981 | 13.690 | 1.00 | 11.72 | O |
| ATOM | 939 | N | ALA | A | 138 | 113.049 | 77.366 | 11.977 | 1.00 | 10.21 | N |
| ATOM | 940 | CA | ALA | A | 138 | 112.696 | 78.596 | 12.691 | 1.00 | 9.82 | C |
| ATOM | 941 | CB | ALA | A | 138 | 113.685 | 79.681 | 12.387 | 1.00 | 9.74 | C |
| ATOM | 942 | C | ALA | A | 138 | 111.312 | 79.035 | 12.289 | 1.00 | 8.83 | C |
| ATOM | 943 | O | ALA | A | 138 | 110.770 | 78.607 | 11.274 | 1.00 | 9.56 | O |
| ATOM | 944 | N | MET | A | 139 | 110.694 | 79.864 | 13.108 | 1.00 | 8.81 | N |
| ATOM | 945 | CA | MET | A | 139 | 109.386 | 80.361 | 12.772 | 1.00 | 8.84 | C |
| ATOM | 946 | CB | MET | A | 139 | 108.344 | 79.295 | 13.087 | 1.00 | 9.71 | C |
| ATOM | 947 | CG | MET | A | 139 | 108.265 | 78.986 | 14.555 | 1.00 | 11.78 | C |
| ATOM | 948 | SD | MET | A | 139 | 107.146 | 77.596 | 14.927 | 1.00 | 13.15 | S |
| ATOM | 949 | CE | MET | A | 139 | 108.135 | 76.231 | 14.353 | 1.00 | 13.23 | C |
| ATOM | 950 | C | MET | A | 139 | 109.107 | 81.656 | 13.505 | 1.00 | 8.40 | C |
| ATOM | 951 | O | MET | A | 139 | 109.776 | 81.999 | 14.469 | 1.00 | 9.27 | O |
| ATOM | 952 | N | GLY | A | 140 | 108.110 | 82.386 | 13.025 | 1.00 | 9.36 | N |
| ATOM | 953 | CA | GLY | A | 140 | 107.696 | 83.616 | 13.670 | 1.00 | 9.28 | C |
| ATOM | 954 | C | GLY | A | 140 | 106.864 | 84.519 | 12.772 | 1.00 | 9.58 | C |
| ATOM | 955 | O | GLY | A | 140 | 106.713 | 84.264 | 11.568 | 1.00 | 9.81 | O |
| ATOM | 956 | N | TRP | A | 141 | 106.334 | 85.584 | 13.377 | 1.00 | 9.33 | N |
| ATOM | 957 | CA | TRP | A | 141 | 105.545 | 86.583 | 12.709 | 1.00 | 8.60 | C |
| ATOM | 958 | CB | TRP | A | 141 | 104.315 | 86.916 | 13.547 | 1.00 | 8.74 | C |
| ATOM | 959 | CG | TRP | A | 141 | 103.164 | 85.907 | 13.476 | 1.00 | 7.54 | C |
| ATOM | 960 | CD1 | TRP | A | 141 | 102.256 | 85.746 | 12.452 | 1.00 | 9.64 | C |
| ATOM | 961 | NE1 | TRP | A | 141 | 101.331 | 84.780 | 12.779 | 1.00 | 9.09 | N |
| ATOM | 962 | CE2 | TRP | A | 141 | 101.627 | 84.288 | 14.016 | 1.00 | 7.98 | C |
| ATOM | 963 | CD2 | TRP | A | 141 | 102.772 | 84.987 | 14.489 | 1.00 | 7.39 | C |
| ATOM | 964 | CE3 | TRP | A | 141 | 103.290 | 84.637 | 15.738 | 1.00 | 7.76 | C |
| ATOM | 965 | CZ3 | TRP | A | 141 | 102.662 | 83.694 | 16.478 | 1.00 | 8.84 | C |
| ATOM | 966 | CH2 | TRP | A | 141 | 101.535 | 83.022 | 15.987 | 1.00 | 9.16 | C |
| ATOM | 967 | CZ2 | TRP | A | 141 | 101.002 | 83.315 | 14.769 | 1.00 | 8.80 | C |
| ATOM | 968 | C | TRP | A | 141 | 106.331 | 87.901 | 12.512 | 1.00 | 9.22 | C |
| ATOM | 969 | O | TRP | A | 141 | 105.751 | 88.942 | 12.195 | 1.00 | 9.68 | O |
| ATOM | 970 | N | GLY | A | 142 | 107.643 | 87.854 | 12.687 | 1.00 | 9.31 | N |
| ATOM | 971 | CA | GLY | A | 142 | 108.457 | 89.036 | 12.521 | 1.00 | 9.66 | C |
| ATOM | 972 | C | GLY | A | 142 | 108.593 | 89.502 | 11.081 | 1.00 | 9.91 | C |
| ATOM | 973 | O | GLY | A | 142 | 107.969 | 88.973 | 10.148 | 1.00 | 9.41 | O |
| ATOM | 974 | N | LEU | A | 143 | 109.452 | 90.508 | 10.910 | 1.00 | 10.96 | N |
| ATOM | 975 | CA | LEU | A | 143 | 109.582 | 91.200 | 9.655 | 1.00 | 12.34 | C |
| ATOM | 976 | CB | LEU | A | 143 | 110.662 | 92.285 | 9.720 | 1.00 | 12.01 | C |
| ATOM | 977 | CG | LEU | A | 143 | 110.556 | 93.463 | 10.677 | 1.00 | 15.15 | C |
| ATOM | 978 | CD1 | LEU | A | 143 | 111.692 | 94.461 | 10.318 | 1.00 | 17.95 | C |
| ATOM | 979 | CD2 | LEU | A | 143 | 109.198 | 94.143 | 10.595 | 1.00 | 14.26 | C |
| ATOM | 980 | C | LEU | A | 143 | 109.947 | 90.221 | 8.539 | 1.00 | 12.86 | C |
| ATOM | 981 | O | LEU | A | 143 | 110.781 | 89.317 | 8.711 | 1.00 | 11.65 | O |
| ATOM | 982 | N | LEU | A | 144 | 109.326 | 90.447 | 7.390 | 1.00 | 12.51 | N |
| ATOM | 983 | CA | LEU | A | 144 | 109.512 | 89.620 | 6.207 | 1.00 | 13.36 | C |
| ATOM | 984 | CB | LEU | A | 144 | 108.286 | 89.658 | 5.294 | 1.00 | 13.94 | C |
| ATOM | 985 | CG | LEU | A | 144 | 106.983 | 88.992 | 5.796 | 1.00 | 14.60 | C |
| ATOM | 986 | CD1 | LEU | A | 144 | 105.720 | 89.403 | 4.980 | 1.00 | 13.70 | C |
| ATOM | 987 | CD2 | LEU | A | 144 | 107.129 | 87.493 | 5.756 | 1.00 | 14.81 | C |
| ATOM | 988 | C | LEU | A | 144 | 110.728 | 90.129 | 5.437 | 1.00 | 13.27 | C |
| ATOM | 989 | O | LEU | A | 144 | 111.151 | 89.508 | 4.509 | 1.00 | 12.04 | O |
| ATOM | 990 | N | GLY | A | 145 | 111.255 | 91.274 | 5.842 | 1.00 | 14.26 | N |
| ATOM | 991 | CA | GLY | A | 145 | 112.358 | 91.903 | 5.141 | 1.00 | 16.85 | C |
| ATOM | 992 | C | GLY | A | 145 | 112.436 | 93.383 | 5.451 | 1.00 | 18.41 | C |
| ATOM | 993 | O | GLY | A | 145 | 111.549 | 93.936 | 6.076 | 1.00 | 18.34 | O |
| ATOM | 994 | N | ARG | A | 147 | 113.522 | 94.021 | 5.045 | 1.00 | 21.32 | N |
| ATOM | 995 | CA | ARG | A | 147 | 113.685 | 95.471 | 5.223 | 1.00 | 23.59 | C |
| ATOM | 996 | CB | ARG | A | 147 | 115.001 | 95.916 | 4.562 | 1.00 | 24.94 | C |
| ATOM | 997 | CG | ARG | A | 147 | 115.455 | 97.365 | 4.855 | 1.00 | 28.99 | C |
| ATOM | 998 | CD | ARG | A | 147 | 116.888 | 97.668 | 4.346 | 1.00 | 32.42 | C |
| ATOM | 999 | NE | ARG | A | 147 | 117.057 | 97.415 | 2.890 | 1.00 | 37.81 | N |
| ATOM | 1000 | CZ | ARG | A | 147 | 118.166 | 96.913 | 2.294 | 1.00 | 39.82 | C |
| ATOM | 1001 | NH1 | ARG | A | 147 | 119.243 | 96.591 | 2.994 | 1.00 | 42.13 | N |
| ATOM | 1002 | NH2 | ARG | A | 147 | 118.207 | 96.755 | 0.977 | 1.00 | 41.82 | N |
| ATOM | 1003 | C | ARG | A | 147 | 112.513 | 96.204 | 4.570 | 1.00 | 23.61 | C |
| ATOM | 1004 | O | ARG | A | 147 | 112.225 | 95.979 | 3.394 | 1.00 | 23.11 | O |
| ATOM | 1005 | N | ASN | A | 148 | 111.831 | 97.051 | 5.343 | 1.00 | 24.41 | N |
| ATOM | 1006 | CA | ASN | A | 148 | 110.649 | 97.781 | 4.869 | 1.00 | 25.07 | C |
| ATOM | 1007 | CB | ASN | A | 148 | 111.006 | 98.770 | 3.753 | 1.00 | 25.20 | C |
| ATOM | 1008 | CG | ASN | A | 148 | 111.691 | 100.035 | 4.259 | 1.00 | 27.29 | C |
| ATOM | 1009 | OD1 | ASN | A | 148 | 112.660 | 100.483 | 3.633 | 1.00 | 23.59 | O |
| ATOM | 1010 | ND2 | ASN | A | 148 | 111.168 | 100.644 | 5.366 | 1.00 | 24.62 | N |
| ATOM | 1011 | C | ASN | A | 148 | 109.527 | 96.893 | 4.346 | 1.00 | 23.61 | C |
| ATOM | 1012 | O | ASN | A | 148 | 108.767 | 97.331 | 3.497 | 1.00 | 25.87 | O |
| ATOM | 1013 | N | ARG | A | 149 | 109.404 | 95.659 | 4.823 | 1.00 | 21.73 | N |
| ATOM | 1014 | CA | ARG | A | 149 | 108.279 | 94.829 | 4.427 | 1.00 | 20.32 | C |
| ATOM | 1015 | CB | ARG | A | 149 | 108.787 | 93.495 | 3.927 | 1.00 | 20.81 | C |
| ATOM | 1016 | CG | ARG | A | 149 | 109.455 | 93.640 | 2.562 | 1.00 | 21.91 | C |
| ATOM | 1017 | CD | ARG | A | 149 | 109.895 | 92.371 | 1.982 | 1.00 | 22.77 | C |
| ATOM | 1018 | NE | ARG | A | 149 | 108.775 | 91.478 | 1.702 | 1.00 | 21.89 | N |
| ATOM | 1019 | CZ | ARG | A | 149 | 108.953 | 90.256 | 1.213 | 1.00 | 23.95 | C |
| ATOM | 1020 | NH1 | ARG | A | 149 | 110.175 | 89.814 | 0.957 | 1.00 | 21.93 | N |
| ATOM | 1021 | NH2 | ARG | A | 149 | 107.909 | 89.480 | 0.960 | 1.00 | 24.30 | N |
| ATOM | 1022 | C | ARG | A | 149 | 107.275 | 94.602 | 5.537 | 1.00 | 19.05 | C |
| ATOM | 1023 | O | ARG | A | 149 | 106.217 | 94.041 | 5.309 | 1.00 | 21.78 | O |
| ATOM | 1024 | N | GLY | A | 150 | 107.602 | 95.000 | 6.738 | 1.00 | 17.50 | N |
| ATOM | 1025 | CA | GLY | A | 150 | 106.691 | 94.810 | 7.859 | 1.00 | 16.77 | C |
| ATOM | 1026 | C | GLY | A | 150 | 106.556 | 93.391 | 8.431 | 1.00 | 15.54 | C |
| ATOM | 1027 | O | GLY | A | 150 | 107.074 | 92.427 | 7.894 | 1.00 | 14.03 | O |
| ATOM | 1028 | N | ILE | A | 151 | 105.870 | 93.281 | 9.557 | 1.00 | 13.01 | N |
| ATOM | 1029 | CA | ILE | A | 151 | 105.650 | 91.973 | 10.142 | 1.00 | 13.62 | C |
| ATOM | 1030 | CB | ILE | A | 151 | 105.276 | 91.988 | 11.666 | 1.00 | 13.81 | C |
| ATOM | 1031 | CG1 | ILE | A | 151 | 103.854 | 92.393 | 11.929 | 1.00 | 16.60 | C |
| ATOM | 1032 | CD1 | ILE | A | 151 | 103.688 | 93.786 | 11.719 | 1.00 | 23.64 | C |
| ATOM | 1033 | CG2 | ILE | A | 151 | 106.244 | 92.821 | 12.541 | 1.00 | 13.35 | C |
| ATOM | 1034 | C | ILE | A | 151 | 104.690 | 91.142 | 9.319 | 1.00 | 12.75 | C |
| ATOM | 1035 | O | ILE | A | 151 | 103.828 | 91.655 | 8.581 | 1.00 | 12.12 | O |
| ATOM | 1036 | N | ALA | A | 152 | 104.842 | 89.837 | 9.459 | 1.00 | 12.05 | N |
| ATOM | 1037 | CA | ALA | A | 152 | 104.043 | 88.882 | 8.716 | 1.00 | 12.02 | C |
| ATOM | 1038 | CB | ALA | A | 152 | 104.742 | 87.564 | 8.723 | 1.00 | 13.09 | C |
| ATOM | 1039 | C | ALA | A | 152 | 102.644 | 88.722 | 9.318 | 1.00 | 13.87 | C |
| ATOM | 1040 | O | ALA | A | 152 | 102.491 | 88.567 | 10.512 | 1.00 | 14.71 | O |
| ATOM | 1041 | N | SER | A | 153 | 101.632 | 88.791 | 8.466 | 1.00 | 12.87 | N |
| ATOM | 1042 | CA | SER | A | 153 | 100.274 | 88.361 | 8.820 | 1.00 | 13.56 | C |
| ATOM | 1043 | CB | SER | A | 153 | 99.380 | 88.608 | 7.622 | 1.00 | 14.10 | C |
| ATOM | 1044 | OG | SER | A | 153 | 98.976 | 89.926 | 7.697 | 1.00 | 20.20 | O |
| ATOM | 1045 | C | SER | A | 153 | 100.179 | 86.890 | 9.186 | 1.00 | 12.01 | C |
| ATOM | 1046 | O | SER | A | 153 | 99.724 | 86.540 | 10.271 | 1.00 | 12.14 | O |
| ATOM | 1047 | N | VAL | A | 154 | 100.668 | 86.037 | 8.307 | 1.00 | 11.05 | N |
| ATOM | 1048 | CA | VAL | A | 154 | 100.584 | 84.589 | 8.506 | 1.00 | 11.10 | C |
| ATOM | 1049 | CB | VAL | A | 154 | 100.192 | 83.844 | 7.198 | 1.00 | 11.95 | C |
| ATOM | 1050 | CG1 | VAL | A | 154 | 100.200 | 82.314 | 7.402 | 1.00 | 12.35 | C |
| ATOM | 1051 | CG2 | VAL | A | 154 | 98.810 | 84.278 | 6.733 | 1.00 | 15.42 | C |
| ATOM | 1052 | C | VAL | A | 154 | 101.915 | 84.093 | 9.046 | 1.00 | 10.21 | C |
| ATOM | 1053 | O | VAL | A | 154 | 102.964 | 84.466 | 8.544 | 1.00 | 9.10 | O |
| ATOM | 1054 | N | LEU | A | 155 | 101.848 | 83.261 | 10.088 | 1.00 | 10.77 | N |
| ATOM | 1055 | CA | LEU | A | 155 | 103.014 | 82.605 | 10.663 | 1.00 | 11.11 | C |
| ATOM | 1056 | CB | LEU | A | 155 | 102.603 | 81.423 | 11.530 | 1.00 | 11.28 | C |
| ATOM | 1057 | CG | LEU | A | 155 | 103.712 | 80.694 | 12.273 | 1.00 | 11.03 | C |
| ATOM | 1058 | CD1 | LEU | A | 155 | 104.469 | 81.652 | 13.230 | 1.00 | 10.57 | C |
| ATOM | 1059 | CD2 | LEU | A | 155 | 103.077 | 79.502 | 13.055 | 1.00 | 10.73 | C |
| ATOM | 1060 | C | LEU | A | 155 | 103.891 | 82.105 | 9.537 | 1.00 | 10.28 | C |
| ATOM | 1061 | O | LEU | A | 155 | 103.413 | 81.448 | 8.616 | 1.00 | 9.82 | O |
| ATOM | 1062 | N | GLN | A | 156 | 105.171 | 82.444 | 9.627 | 1.00 | 10.99 | N |
| ATOM | 1063 | CA | GLN | A | 156 | 106.193 | 81.977 | 8.697 | 1.00 | 9.90 | C |
| ATOM | 1064 | CB | GLN | A | 156 | 107.060 | 83.147 | 8.224 | 1.00 | 11.17 | C |
| ATOM | 1065 | CG | GLN | A | 156 | 106.311 | 84.356 | 7.675 | 1.00 | 11.08 | C |
| ATOM | 1066 | CD | GLN | A | 156 | 105.696 | 84.144 | 6.305 | 1.00 | 13.46 | C |
| ATOM | 1067 | OE1 | GLN | A | 156 | 104.479 | 84.307 | 6.144 | 1.00 | 18.04 | O |
| ATOM | 1068 | NE2 | GLN | A | 156 | 106.526 | 83.852 | 5.297 | 1.00 | 12.69 | N |
| ATOM | 1069 | C | GLN | A | 156 | 107.109 | 80.911 | 9.314 | 1.00 | 10.38 | C |
| ATOM | 1070 | O | GLN | A | 156 | 107.325 | 80.890 | 10.500 | 1.00 | 9.15 | O |
| ATOM | 1071 | N | GLU | A | 157 | 107.622 | 80.023 | 8.484 | 1.00 | 10.58 | N |
| ATOM | 1072 | CA | GLU | A | 157 | 108.624 | 79.039 | 8.902 | 1.00 | 11.44 | C |
| ATOM | 1073 | CB | GLU | A | 157 | 108.009 | 77.640 | 9.084 | 1.00 | 11.97 | C |
| ATOM | 1074 | CG | GLU | A | 157 | 107.519 | 77.013 | 7.779 | 1.00 | 13.37 | C |
| ATOM | 1075 | CD | GLU | A | 157 | 106.827 | 75.684 | 7.985 | 1.00 | 17.17 | C |
| ATOM | 1076 | OE1 | GLU | A | 157 | 105.820 | 75.645 | 8.721 | 1.00 | 14.74 | O |
| ATOM | 1077 | OE2 | GLU | A | 157 | 107.295 | 74.678 | 7.405 | 1.00 | 17.86 | O |
| ATOM | 1078 | C | GLU | A | 157 | 109.777 | 79.028 | 7.882 | 1.00 | 12.08 | C |
| ATOM | 1079 | O | GLU | A | 157 | 109.642 | 79.488 | 6.744 | 1.00 | 11.97 | O |
| ATOM | 1080 | N | LEU | A | 158 | 110.915 | 78.515 | 8.324 | 1.00 | 12.03 | N |
| ATOM | 1081 | CA | LEU | A | 158 | 112.167 | 78.609 | 7.571 | 1.00 | 11.77 | C |
| ATOM | 1082 | CB | LEU | A | 158 | 112.878 | 79.915 | 7.920 | 1.00 | 10.99 | C |
| ATOM | 1083 | CG | LEU | A | 158 | 114.225 | 80.217 | 7.257 | 1.00 | 10.22 | C |
| ATOM | 1084 | CD1 | LEU | A | 158 | 113.994 | 80.449 | 5.775 | 1.00 | 13.15 | C |
| ATOM | 1085 | CD2 | LEU | A | 158 | 114.849 | 81.401 | 7.860 | 1.00 | 9.18 | C |
| ATOM | 1086 | C | LEU | A | 158 | 113.081 | 77.466 | 7.960 | 1.00 | 11.85 | C |
| ATOM | 1087 | O | LEU | A | 158 | 113.387 | 77.339 | 9.136 | 1.00 | 13.32 | O |
| ATOM | 1088 | N | ASN | A | 159 | 113.573 | 76.715 | 6.985 | 1.00 | 13.44 | N |
| ATOM | 1089 | CA | ASN | A | 159 | 114.598 | 75.671 | 7.201 | 1.00 | 14.06 | C |
| ATOM | 1090 | CB | ASN | A | 159 | 114.671 | 74.729 | 5.995 | 1.00 | 15.37 | C |
| ATOM | 1091 | CG | ASN | A | 159 | 113.494 | 73.811 | 5.897 | 1.00 | 18.01 | C |
| ATOM | 1092 | OD1 | ASN | A | 159 | 112.347 | 74.256 | 5.743 | 1.00 | 20.60 | O |
| ATOM | 1093 | ND2 | ASN | A | 159 | 113.773 | 72.505 | 5.934 | 1.00 | 16.64 | N |
| ATOM | 1094 | C | ASN | A | 159 | 115.978 | 76.326 | 7.417 | 1.00 | 14.38 | C |
| ATOM | 1095 | O | ASN | A | 159 | 116.421 | 77.187 | 6.646 | 1.00 | 14.04 | O |
| ATOM | 1096 | N | VAL | A | 160 | 116.632 | 75.929 | 8.489 | 1.00 | 13.06 | N |
| ATOM | 1097 | CA | VAL | A | 160 | 117.813 | 76.555 | 8.988 | 1.00 | 14.97 | C |
| ATOM | 1098 | CB | VAL | A | 160 | 117.222 | 77.421 | 10.121 | 1.00 | 16.48 | C |
| ATOM | 1099 | CG1 | VAL | A | 160 | 117.618 | 77.039 | 11.518 | 1.00 | 14.40 | C |
| ATOM | 1100 | CG2 | VAL | A | 160 | 117.105 | 78.852 | 9.735 | 1.00 | 15.58 | C |
| ATOM | 1101 | C | VAL | A | 160 | 118.777 | 75.409 | 9.392 | 1.00 | 15.54 | C |
| ATOM | 1102 | O | VAL | A | 160 | 118.342 | 74.285 | 9.577 | 1.00 | 16.02 | O |
| ATOM | 1103 | N | THR | A | 162 | 120.061 | 75.723 | 9.437 | 1.00 | 16.70 | N |
| ATOM | 1104 | CA | THR | A | 162 | 121.141 | 74.817 | 9.828 | 1.00 | 16.60 | C |
| ATOM | 1105 | CB | THR | A | 162 | 122.194 | 74.755 | 8.673 | 1.00 | 16.76 | C |
| ATOM | 1106 | OG1 | THR | A | 162 | 121.617 | 74.114 | 7.544 | 1.00 | 17.43 | O |
| ATOM | 1107 | CG2 | THR | A | 162 | 123.406 | 73.863 | 9.050 | 1.00 | 19.45 | C |
| ATOM | 1108 | C | THR | A | 162 | 121.833 | 75.322 | 11.069 | 1.00 | 16.16 | C |
| ATOM | 1109 | O | THR | A | 162 | 122.322 | 76.426 | 11.112 | 1.00 | 15.24 | O |
| ATOM | 1110 | N | VAL | A | 163 | 121.938 | 74.482 | 12.092 | 1.00 | 17.27 | N |
| ATOM | 1111 | CA | VAL | A | 163 | 122.732 | 74.847 | 13.244 | 1.00 | 17.28 | C |
| ATOM | 1112 | CB | VAL | A | 163 | 122.632 | 73.798 | 14.368 | 1.00 | 16.89 | C |
| ATOM | 1113 | CG1 | VAL | A | 163 | 123.545 | 74.190 | 15.465 | 1.00 | 16.28 | C |
| ATOM | 1114 | CG2 | VAL | A | 163 | 121.214 | 73.723 | 14.883 | 1.00 | 18.99 | C |
| ATOM | 1115 | C | VAL | A | 163 | 124.200 | 74.950 | 12.833 | 1.00 | 17.56 | C |
| ATOM | 1116 | O | VAL | A | 163 | 124.672 | 74.070 | 12.142 | 1.00 | 19.28 | O |
| ATOM | 1117 | N | VAL | A | 164 | 124.883 | 76.004 | 13.246 | 1.00 | 19.27 | N |
| ATOM | 1118 | CA | VAL | A | 164 | 126.304 | 76.198 | 12.993 | 1.00 | 20.56 | C |
| ATOM | 1119 | CB | VAL | A | 164 | 126.613 | 77.288 | 11.897 | 1.00 | 20.17 | C |
| ATOM | 1120 | CG1 | VAL | A | 164 | 125.962 | 76.899 | 10.563 | 1.00 | 20.73 | C |
| ATOM | 1121 | CG2 | VAL | A | 164 | 126.210 | 78.694 | 12.378 | 1.00 | 20.94 | C |
| ATOM | 1122 | C | VAL | A | 164 | 127.018 | 76.617 | 14.255 | 1.00 | 21.41 | C |
| ATOM | 1123 | O | VAL | A | 164 | 126.431 | 77.262 | 15.145 | 1.00 | 19.74 | O |
| ATOM | 1124 | N | THR | A | 165 | 128.311 | 76.278 | 14.310 | 1.00 | 21.64 | N |
| ATOM | 1125 | CA | THR | A | 165 | 129.153 | 76.764 | 15.392 | 1.00 | 22.82 | C |
| ATOM | 1126 | CB | THR | A | 165 | 129.927 | 75.581 | 16.026 | 1.00 | 22.38 | C |
| ATOM | 1127 | OG1 | THR | A | 165 | 130.608 | 74.839 | 14.998 | 1.00 | 19.62 | O |
| ATOM | 1128 | CG2 | THR | A | 165 | 128.942 | 74.576 | 16.600 | 1.00 | 22.24 | C |
| ATOM | 1129 | C | THR | A | 165 | 130.083 | 77.844 | 14.925 | 1.00 | 24.15 | C |
| ATOM | 1130 | O | THR | A | 165 | 130.734 | 78.495 | 15.757 | 1.00 | 24.93 | O |
| ATOM | 1131 | N | SER | A | 166 | 130.137 | 78.041 | 13.598 | 1.00 | 26.19 | N |
| ATOM | 1132 | CA | SER | A | 166 | 130.980 | 79.070 | 12.996 | 1.00 | 27.22 | C |
| ATOM | 1133 | CB | SER | A | 166 | 131.048 | 79.015 | 11.443 | 1.00 | 27.84 | C |
| ATOM | 1134 | OG | SER | A | 166 | 130.979 | 77.724 | 10.839 | 1.00 | 29.31 | O |
| ATOM | 1135 | C | SER | A | 166 | 130.326 | 80.348 | 13.353 | 1.00 | 26.94 | C |
| ATOM | 1136 | O | SER | A | 166 | 129.114 | 80.486 | 13.126 | 1.00 | 27.10 | O |
| ATOM | 1137 | N | LEU | A | 167 | 131.114 | 81.276 | 13.884 | 1.00 | 25.98 | N |
| ATOM | 1138 | CA | LEU | A | 167 | 130.692 | 82.625 | 14.176 | 1.00 | 25.96 | C |
| ATOM | 1139 | CB | LEU | A | 167 | 130.217 | 83.263 | 12.864 | 1.00 | 26.48 | C |
| ATOM | 1140 | CG | LEU | A | 167 | 130.244 | 84.777 | 12.596 | 1.00 | 26.47 | C |
| ATOM | 1141 | CD1 | LEU | A | 167 | 130.993 | 85.613 | 13.558 | 1.00 | 28.26 | C |
| ATOM | 1142 | CD2 | LEU | A | 167 | 130.665 | 85.077 | 11.137 | 1.00 | 22.91 | C |
| ATOM | 1143 | C | LEU | A | 167 | 129.653 | 82.659 | 15.318 | 1.00 | 26.53 | C |
| ATOM | 1144 | O | LEU | A | 167 | 128.755 | 83.543 | 15.407 | 1.00 | 26.07 | O |
| ATOM | 1145 | N | CYS | A | 168 | 129.821 | 81.699 | 16.228 | 1.00 | 25.69 | N |
| ATOM | 1146 | CA | CYS | A | 168 | 128.925 | 81.537 | 17.359 | 1.00 | 24.68 | C |
| ATOM | 1147 | CB | CYS | A | 168 | 128.196 | 80.201 | 17.206 | 1.00 | 23.95 | C |
| ATOM | 1148 | SG | CYS | A | 168 | 126.666 | 80.129 | 18.161 | 1.00 | 22.30 | S |
| ATOM | 1149 | C | CYS | A | 168 | 129.716 | 81.623 | 18.673 | 1.00 | 25.08 | C |
| ATOM | 1150 | O | CYS | A | 168 | 130.947 | 81.705 | 18.659 | 1.00 | 25.00 | O |
| ATOM | 1151 | N | ARG | A | 177 | 129.001 | 81.684 | 19.790 | 1.00 | 24.67 | N |
| ATOM | 1152 | CA | ARG | A | 177 | 129.580 | 81.482 | 21.127 | 1.00 | 24.31 | C |
| ATOM | 1153 | CB | ARG | A | 177 | 129.570 | 82.767 | 21.934 | 1.00 | 25.30 | C |
| ATOM | 1154 | CG | ARG | A | 177 | 128.281 | 83.498 | 21.874 | 1.00 | 24.14 | C |
| ATOM | 1155 | CD | ARG | A | 177 | 128.286 | 84.837 | 22.536 | 1.00 | 24.36 | C |
| ATOM | 1156 | NE | ARG | A | 177 | 126.900 | 85.258 | 22.760 | 1.00 | 22.19 | N |
| ATOM | 1157 | CZ | ARG | A | 177 | 126.527 | 86.408 | 23.307 | 1.00 | 21.16 | C |
| ATOM | 1158 | NH1 | ARG | A | 177 | 127.411 | 87.276 | 23.731 | 1.00 | 23.53 | N |
| ATOM | 1159 | NH2 | ARG | A | 177 | 125.233 | 86.670 | 23.471 | 1.00 | 22.41 | N |
| ATOM | 1160 | C | ARG | A | 177 | 128.843 | 80.368 | 21.865 | 1.00 | 23.44 | C |
| ATOM | 1161 | O | ARG | A | 177 | 127.757 | 79.958 | 21.440 | 1.00 | 22.40 | O |
| ATOM | 1162 | N | ARG | A | 178 | 129.490 | 79.823 | 22.907 | 1.00 | 22.01 | N |
| ATOM | 1163 | CA | ARG | A | 178 | 128.972 | 78.673 | 23.639 | 1.00 | 22.03 | C |
| ATOM | 1164 | CB | ARG | A | 178 | 129.914 | 78.274 | 24.807 | 1.00 | 22.48 | C |
| ATOM | 1165 | CG | ARG | A | 178 | 131.255 | 77.692 | 24.382 | 1.00 | 27.01 | C |
| ATOM | 1166 | CD | ARG | A | 178 | 132.106 | 77.144 | 25.540 | 1.00 | 32.00 | C |
| ATOM | 1167 | NE | ARG | A | 178 | 131.358 | 77.038 | 26.807 | 1.00 | 36.53 | N |
| ATOM | 1168 | CZ | ARG | A | 178 | 131.302 | 75.966 | 27.614 | 1.00 | 40.52 | C |
| ATOM | 1169 | NH1 | ARG | A | 178 | 131.952 | 74.830 | 27.337 | 1.00 | 42.48 | N |
| ATOM | 1170 | NH2 | ARG | A | 178 | 130.590 | 76.044 | 28.734 | 1.00 | 41.32 | N |
| ATOM | 1171 | C | ARG | A | 178 | 127.607 | 78.951 | 24.241 | 1.00 | 19.50 | C |
| ATOM | 1172 | O | ARG | A | 178 | 126.820 | 78.031 | 24.418 | 1.00 | 19.14 | O |
| ATOM | 1173 | N | SER | A | 179 | 127.368 | 80.214 | 24.576 | 1.00 | 19.13 | N |
| ATOM | 1174 | CA | SER | A | 179 | 126.120 | 80.657 | 25.223 | 1.00 | 18.59 | C |
| ATOM | 1175 | CB | SER | A | 179 | 126.401 | 81.936 | 25.997 | 1.00 | 18.72 | C |
| ATOM | 1176 | OG | SER | A | 179 | 126.882 | 82.967 | 25.131 | 1.00 | 22.47 | O |
| ATOM | 1177 | C | SER | A | 179 | 124.913 | 80.878 | 24.283 | 1.00 | 17.76 | C |
| ATOM | 1178 | O | SER | A | 179 | 123.884 | 81.425 | 24.721 | 1.00 | 16.75 | O |
| ATOM | 1179 | N | ASN | A | 180 | 125.048 | 80.485 | 23.011 | 1.00 | 16.98 | N |
| ATOM | 1180 | CA | ASN | A | 180 | 123.970 | 80.565 | 22.010 | 1.00 | 16.70 | C |
| ATOM | 1181 | CB | ASN | A | 180 | 124.156 | 81.782 | 21.062 | 1.00 | 15.93 | C |
| ATOM | 1182 | CG | ASN | A | 180 | 123.755 | 83.073 | 21.691 | 1.00 | 14.93 | C |
| ATOM | 1183 | OD1 | ASN | A | 180 | 124.590 | 83.948 | 21.924 | 1.00 | 12.89 | O |
| ATOM | 1184 | ND2 | ASN | A | 180 | 122.443 | 83.238 | 21.945 | 1.00 | 12.93 | N |
| ATOM | 1185 | C | ASN | A | 180 | 123.901 | 79.319 | 21.178 | 1.00 | 16.70 | C |
| ATOM | 1186 | O | ASN | A | 180 | 124.922 | 78.648 | 20.982 | 1.00 | 17.32 | O |
| ATOM | 1187 | N | VAL | A | 181 | 122.710 | 78.977 | 20.680 | 1.00 | 14.62 | N |
| ATOM | 1188 | CA | VAL | A | 181 | 122.599 | 78.098 | 19.543 | 1.00 | 14.11 | C |
| ATOM | 1189 | CB | VAL | A | 181 | 121.335 | 77.184 | 19.607 | 1.00 | 14.56 | C |
| ATOM | 1190 | CG1 | VAL | A | 181 | 121.473 | 76.177 | 20.706 | 1.00 | 14.01 | C |
| ATOM | 1191 | CG2 | VAL | A | 181 | 121.103 | 76.448 | 18.303 | 1.00 | 14.16 | C |
| ATOM | 1192 | C | VAL | A | 181 | 122.560 | 79.051 | 18.337 | 1.00 | 14.38 | C |
| ATOM | 1193 | O | VAL | A | 181 | 121.815 | 80.028 | 18.356 | 1.00 | 12.39 | O |
| ATOM | 1194 | N | CYS | A | 182 | 123.395 | 78.809 | 17.328 | 1.00 | 14.74 | N |
| ATOM | 1195 | CA | CYS | A | 182 | 123.415 | 79.710 | 16.165 | 1.00 | 15.25 | C |
| ATOM | 1196 | CB | CYS | A | 182 | 124.786 | 80.340 | 15.928 | 1.00 | 14.83 | C |
| ATOM | 1197 | SG | CYS | A | 182 | 125.389 | 81.395 | 17.194 | 1.00 | 18.14 | S |
| ATOM | 1198 | C | CYS | A | 182 | 122.933 | 78.959 | 14.960 | 1.00 | 14.25 | C |
| ATOM | 1199 | O | CYS | A | 182 | 123.086 | 77.735 | 14.870 | 1.00 | 15.09 | O |
| ATOM | 1200 | N | THR | A | 183 | 122.242 | 79.662 | 14.045 | 1.00 | 14.78 | N |
| ATOM | 1201 | CA | THR | A | 183 | 121.743 | 79.034 | 12.815 | 1.00 | 13.29 | C |
| ATOM | 1202 | CB | THR | A | 183 | 120.257 | 78.747 | 12.830 | 1.00 | 14.18 | C |
| ATOM | 1203 | OG1 | THR | A | 183 | 119.524 | 79.972 | 12.915 | 1.00 | 12.19 | O |
| ATOM | 1204 | CG2 | THR | A | 183 | 119.848 | 77.939 | 14.071 | 1.00 | 12.83 | C |
| ATOM | 1205 | C | THR | A | 183 | 121.991 | 79.912 | 11.598 | 1.00 | 13.25 | C |
| ATOM | 1206 | O | THR | A | 183 | 122.105 | 81.130 | 11.704 | 1.00 | 13.45 | O |
| ATOM | 1207 | N | LEU | A | 184 | 122.089 | 79.240 | 10.459 | 1.00 | 12.74 | N |
| ATOM | 1208 | CA | LEU | A | 184 | 122.323 | 79.871 | 9.165 | 1.00 | 13.08 | C |
| ATOM | 1209 | CB | LEU | A | 184 | 123.817 | 79.869 | 8.840 | 1.00 | 13.41 | C |
| ATOM | 1210 | CG | LEU | A | 184 | 124.277 | 80.676 | 7.616 | 1.00 | 12.50 | C |
| ATOM | 1211 | CD1 | LEU | A | 184 | 123.904 | 82.088 | 7.832 | 1.00 | 13.61 | C |
| ATOM | 1212 | CD2 | LEU | A | 184 | 125.802 | 80.586 | 7.403 | 1.00 | 15.81 | C |
| ATOM | 1213 | C | LEU | A | 184 | 121.550 | 79.148 | 8.056 | 1.00 | 13.01 | C |
| ATOM | 1214 | O | LEU | A | 184 | 121.424 | 77.930 | 8.064 | 1.00 | 14.79 | O |
| ATOM | 1215 | N | VAL | A | 185 | 121.005 | 79.924 | 7.116 | 1.00 | 13.04 | N |
| ATOM | 1216 | CA | VAL | A | 185 | 120.481 | 79.403 | 5.883 | 1.00 | 13.22 | C |
| ATOM | 1217 | CB | VAL | A | 185 | 119.306 | 80.244 | 5.312 | 1.00 | 12.79 | C |
| ATOM | 1218 | CG1 | VAL | A | 185 | 118.872 | 79.637 | 3.971 | 1.00 | 12.35 | C |
| ATOM | 1219 | CG2 | VAL | A | 185 | 118.148 | 80.288 | 6.310 | 1.00 | 12.88 | C |
| ATOM | 1220 | C | VAL | A | 185 | 121.658 | 79.500 | 4.912 | 1.00 | 14.51 | C |
| ATOM | 1221 | O | VAL | A | 185 | 122.080 | 80.592 | 4.568 | 1.00 | 15.25 | O |
| ATOM | 1222 | N | ARG | A | 186 | 122.226 | 78.351 | 4.552 | 1.00 | 15.87 | N |
| ATOM | 1223 | CA | ARG | A | 186 | 123.434 | 78.322 | 3.735 | 1.00 | 17.17 | C |
| ATOM | 1224 | CB | ARG | A | 186 | 124.126 | 76.968 | 3.880 | 1.00 | 17.06 | C |
| ATOM | 1225 | CG | ARG | A | 186 | 124.599 | 76.718 | 5.336 | 1.00 | 15.58 | C |
| ATOM | 1226 | CD | ARG | A | 186 | 125.078 | 75.332 | 5.600 | 1.00 | 16.03 | C |
| ATOM | 1227 | NE | ARG | A | 186 | 126.014 | 75.267 | 6.716 | 1.00 | 13.98 | N |
| ATOM | 1228 | CZ | ARG | A | 186 | 126.463 | 74.133 | 7.217 | 1.00 | 17.51 | C |
| ATOM | 1229 | NH1 | ARG | A | 186 | 126.038 | 72.957 | 6.725 | 1.00 | 16.71 | N |
| ATOM | 1230 | NH2 | ARG | A | 186 | 127.321 | 74.153 | 8.237 | 1.00 | 14.81 | N |
| ATOM | 1231 | C | ARG | A | 186 | 123.015 | 78.576 | 2.300 | 1.00 | 17.82 | C |
| ATOM | 1232 | O | ARG | A | 186 | 122.028 | 78.026 | 1.842 | 1.00 | 17.00 | O |
| ATOM | 1233 | N | GLY | A | 186A | 123.720 | 79.462 | 1.627 | 1.00 | 19.58 | N |
| ATOM | 1234 | CA | GLY | A | 186A | 123.554 | 79.603 | 0.180 | 1.00 | 20.40 | C |
| ATOM | 1235 | C | GLY | A | 186A | 122.547 | 80.647 | −0.286 | 1.00 | 20.64 | C |
| ATOM | 1236 | O | GLY | A | 186A | 122.424 | 80.872 | −1.488 | 1.00 | 19.85 | O |
| ATOM | 1237 | N | ARG | A | 187 | 121.812 | 81.259 | 0.636 | 1.00 | 19.88 | N |
| ATOM | 1238 | CA | ARG | A | 187 | 120.994 | 82.418 | 0.289 | 1.00 | 20.95 | C |
| ATOM | 1239 | CB | ARG | A | 187 | 119.660 | 82.005 | −0.328 | 1.00 | 21.24 | C |
| ATOM | 1240 | CG | ARG | A | 187 | 118.872 | 81.043 | 0.501 | 1.00 | 21.57 | C |
| ATOM | 1241 | CD | ARG | A | 187 | 117.595 | 80.569 | −0.149 | 1.00 | 23.44 | C |
| ATOM | 1242 | NE | ARG | A | 187 | 116.714 | 80.057 | 0.890 | 1.00 | 22.68 | N |
| ATOM | 1243 | CZ | ARG | A | 187 | 115.616 | 80.663 | 1.329 | 1.00 | 22.51 | C |
| ATOM | 1244 | NH1 | ARG | A | 187 | 115.211 | 81.819 | 0.825 | 1.00 | 18.82 | N |
| ATOM | 1245 | NH2 | ARG | A | 187 | 114.931 | 80.088 | 2.315 | 1.00 | 24.92 | N |
| ATOM | 1246 | C | ARG | A | 187 | 120.775 | 83.288 | 1.495 | 1.00 | 19.97 | C |
| ATOM | 1247 | O | ARG | A | 187 | 121.103 | 82.885 | 2.607 | 1.00 | 22.01 | O |
| ATOM | 1248 | N | GLN | A | 188 | 120.304 | 84.499 | 1.276 | 1.00 | 17.87 | N |
| ATOM | 1249 | CA | GLN | A | 188 | 120.066 | 85.433 | 2.360 | 1.00 | 16.49 | C |
| ATOM | 1250 | CB | GLN | A | 188 | 120.214 | 86.871 | 1.879 | 1.00 | 17.01 | C |
| ATOM | 1251 | CG | GLN | A | 188 | 121.674 | 87.280 | 1.731 | 1.00 | 17.83 | C |
| ATOM | 1252 | CD | GLN | A | 188 | 121.832 | 88.649 | 1.152 | 1.00 | 23.28 | C |
| ATOM | 1253 | OE1 | GLN | A | 188 | 121.811 | 88.808 | −0.058 | 1.00 | 28.17 | O |
| ATOM | 1254 | NE2 | GLN | A | 188 | 122.000 | 89.642 | 2.003 | 1.00 | 23.92 | N |
| ATOM | 1255 | C | GLN | A | 188 | 118.653 | 85.217 | 2.919 | 1.00 | 16.77 | C |
| ATOM | 1256 | O | GLN | A | 188 | 117.667 | 85.560 | 2.283 | 1.00 | 14.78 | O |
| ATOM | 1257 | N | ALA | A | 188A | 118.585 | 84.646 | 4.119 | 1.00 | 14.76 | N |
| ATOM | 1258 | CA | ALA | A | 188A | 117.325 | 84.382 | 4.771 | 1.00 | 13.41 | C |
| ATOM | 1259 | CB | ALA | A | 188A | 116.648 | 83.167 | 4.137 | 1.00 | 13.23 | C |
| ATOM | 1260 | C | ALA | A | 188A | 117.638 | 84.163 | 6.247 | 1.00 | 13.58 | C |
| ATOM | 1261 | O | ALA | A | 188A | 118.777 | 83.794 | 6.589 | 1.00 | 13.78 | O |
| ATOM | 1262 | N | GLY | A | 189 | 116.654 | 84.424 | 7.117 | 1.00 | 11.61 | N |
| ATOM | 1263 | CA | GLY | A | 189 | 116.824 | 84.238 | 8.549 | 1.00 | 9.95 | C |
| ATOM | 1264 | C | GLY | A | 189 | 115.690 | 84.892 | 9.339 | 1.00 | 10.51 | C |
| ATOM | 1265 | O | GLY | A | 189 | 114.676 | 85.406 | 8.760 | 1.00 | 9.22 | O |
| ATOM | 1266 | N | VAL | A | 190 | 115.849 | 84.915 | 10.656 | 1.00 | 8.67 | N |
| ATOM | 1267 | CA | VAL | A | 190 | 114.881 | 85.555 | 11.506 | 1.00 | 9.05 | C |
| ATOM | 1268 | CB | VAL | A | 190 | 114.924 | 85.048 | 12.984 | 1.00 | 8.68 | C |
| ATOM | 1269 | CG1 | VAL | A | 190 | 114.882 | 83.615 | 13.028 | 1.00 | 10.46 | C |
| ATOM | 1270 | CG2 | VAL | A | 190 | 116.123 | 85.559 | 13.724 | 1.00 | 9.81 | C |
| ATOM | 1271 | C | VAL | A | 190 | 115.113 | 87.067 | 11.498 | 1.00 | 9.96 | C |
| ATOM | 1272 | O | VAL | A | 190 | 116.155 | 87.575 | 11.002 | 1.00 | 9.51 | O |
| ATOM | 1273 | N | CYS | A | 191 | 114.135 | 87.793 | 12.010 | 1.00 | 9.81 | N |
| ATOM | 1274 | CA | CYS | A | 191 | 114.216 | 89.246 | 12.006 | 1.00 | 10.31 | C |
| ATOM | 1275 | CB | CYS | A | 191 | 113.804 | 89.752 | 10.621 | 1.00 | 10.87 | C |
| ATOM | 1276 | SG | CYS | A | 191 | 114.307 | 91.432 | 10.168 | 1.00 | 10.84 | S |
| ATOM | 1277 | C | CYS | A | 191 | 113.362 | 89.844 | 13.161 | 1.00 | 10.65 | C |
| ATOM | 1278 | O | CYS | A | 191 | 112.837 | 89.117 | 14.015 | 1.00 | 9.45 | O |
| ATOM | 1279 | N | PHE | A | 192 | 113.240 | 91.166 | 13.198 | 1.00 | 10.29 | N |
| ATOM | 1280 | CA | PHE | A | 192 | 112.624 | 91.863 | 14.336 | 1.00 | 11.42 | C |
| ATOM | 1281 | CB | PHE | A | 192 | 112.695 | 93.397 | 14.135 | 1.00 | 13.12 | C |
| ATOM | 1282 | CG | PHE | A | 192 | 114.097 | 93.905 | 13.934 | 1.00 | 16.73 | C |
| ATOM | 1283 | CD1 | PHE | A | 192 | 114.439 | 94.638 | 12.816 | 1.00 | 22.66 | C |
| ATOM | 1284 | CE1 | PHE | A | 192 | 115.766 | 95.066 | 12.598 | 1.00 | 24.01 | C |
| ATOM | 1285 | CZ | PHE | A | 192 | 116.763 | 94.749 | 13.512 | 1.00 | 26.04 | C |
| ATOM | 1286 | CE2 | PHE | A | 192 | 116.437 | 94.012 | 14.634 | 1.00 | 25.12 | C |
| ATOM | 1287 | CD2 | PHE | A | 192 | 115.099 | 93.579 | 14.833 | 1.00 | 22.89 | C |
| ATOM | 1288 | C | PHE | A | 192 | 111.168 | 91.393 | 14.468 | 1.00 | 10.64 | C |
| ATOM | 1289 | O | PHE | A | 192 | 110.466 | 91.310 | 13.471 | 1.00 | 10.55 | O |
| ATOM | 1290 | N | GLY | A | 193 | 110.720 | 91.120 | 15.687 | 1.00 | 10.02 | N |
| ATOM | 1291 | CA | GLY | A | 193 | 109.411 | 90.502 | 15.895 | 1.00 | 8.83 | C |
| ATOM | 1292 | C | GLY | A | 193 | 109.454 | 88.982 | 15.984 | 1.00 | 8.49 | C |
| ATOM | 1293 | O | GLY | A | 193 | 108.473 | 88.319 | 16.372 | 1.00 | 7.20 | O |
| ATOM | 1294 | N | ASP | A | 194 | 110.573 | 88.392 | 15.586 | 1.00 | 8.15 | N |
| ATOM | 1295 | CA | ASP | A | 194 | 110.732 | 86.948 | 15.733 | 1.00 | 8.91 | C |
| ATOM | 1296 | CB | ASP | A | 194 | 111.617 | 86.339 | 14.643 | 1.00 | 7.38 | C |
| ATOM | 1297 | CG | ASP | A | 194 | 110.945 | 86.281 | 13.316 | 1.00 | 7.95 | C |
| ATOM | 1298 | OD1 | ASP | A | 194 | 109.709 | 86.008 | 13.261 | 1.00 | 8.95 | O |
| ATOM | 1299 | OD2 | ASP | A | 194 | 111.565 | 86.512 | 12.260 | 1.00 | 8.96 | O |
| ATOM | 1300 | C | ASP | A | 194 | 111.317 | 86.586 | 17.076 | 1.00 | 9.20 | C |
| ATOM | 1301 | O | ASP | A | 194 | 111.309 | 85.395 | 17.430 | 1.00 | 9.16 | O |
| ATOM | 1302 | N | SER | A | 195 | 111.885 | 87.570 | 17.782 | 1.00 | 8.94 | N |
| ATOM | 1303 | CA | SER | A | 195 | 112.442 | 87.285 | 19.106 | 1.00 | 9.71 | C |
| ATOM | 1304 | CB | SER | A | 195 | 112.999 | 88.542 | 19.777 | 1.00 | 10.40 | C |
| ATOM | 1305 | OG | SER | A | 195 | 114.209 | 88.980 | 19.086 | 1.00 | 10.55 | O |
| ATOM | 1306 | C | SER | A | 195 | 111.379 | 86.579 | 19.960 | 1.00 | 8.98 | C |
| ATOM | 1307 | O | SER | A | 195 | 110.219 | 86.875 | 19.859 | 1.00 | 9.20 | O |
| ATOM | 1308 | N | GLY | A | 196 | 111.826 | 85.604 | 20.743 | 1.00 | 9.23 | N |
| ATOM | 1309 | CA | GLY | A | 196 | 110.995 | 84.817 | 21.624 | 1.00 | 8.45 | C |
| ATOM | 1310 | C | GLY | A | 196 | 110.439 | 83.557 | 20.995 | 1.00 | 8.46 | C |
| ATOM | 1311 | O | GLY | A | 196 | 109.923 | 82.705 | 21.717 | 1.00 | 9.75 | O |
| ATOM | 1312 | N | SER | A | 197 | 110.568 | 83.402 | 19.680 | 1.00 | 8.42 | N |
| ATOM | 1313 | CA | SER | A | 197 | 109.925 | 82.317 | 18.971 | 1.00 | 7.91 | C |
| ATOM | 1314 | CB | SER | A | 197 | 109.753 | 82.684 | 17.507 | 1.00 | 8.39 | C |
| ATOM | 1315 | OG | SER | A | 197 | 109.087 | 83.943 | 17.307 | 1.00 | 7.99 | O |
| ATOM | 1316 | C | SER | A | 197 | 110.799 | 81.056 | 19.073 | 1.00 | 7.87 | C |
| ATOM | 1317 | O | SER | A | 197 | 112.015 | 81.164 | 19.156 | 1.00 | 8.77 | O |
| ATOM | 1318 | N | PRO | A | 198 | 110.205 | 79.889 | 18.979 | 1.00 | 8.84 | N |
| ATOM | 1319 | CA | PRO | A | 198 | 110.970 | 78.637 | 19.072 | 1.00 | 8.95 | C |
| ATOM | 1320 | CB | PRO | A | 198 | 109.905 | 77.621 | 19.235 | 1.00 | 7.91 | C |
| ATOM | 1321 | CG | PRO | A | 198 | 108.696 | 78.219 | 18.455 | 1.00 | 8.40 | C |
| ATOM | 1322 | CD | PRO | A | 198 | 108.755 | 79.628 | 18.798 | 1.00 | 8.90 | C |
| ATOM | 1323 | C | PRO | A | 198 | 111.814 | 78.284 | 17.836 | 1.00 | 8.54 | C |
| ATOM | 1324 | O | PRO | A | 198 | 111.469 | 78.642 | 16.728 | 1.00 | 8.16 | O |
| ATOM | 1325 | N | LEU | A | 199 | 112.904 | 77.585 | 18.080 | 1.00 | 9.38 | N |
| ATOM | 1326 | CA | LEU | A | 199 | 113.705 | 76.927 | 17.061 | 1.00 | 9.88 | C |
| ATOM | 1327 | CB | LEU | A | 199 | 115.171 | 77.267 | 17.249 | 1.00 | 9.88 | C |
| ATOM | 1328 | CG | LEU | A | 199 | 116.049 | 76.476 | 16.288 | 1.00 | 11.93 | C |
| ATOM | 1329 | CD1 | LEU | A | 199 | 115.928 | 76.997 | 14.853 | 1.00 | 12.39 | C |
| ATOM | 1330 | CD2 | LEU | A | 199 | 117.459 | 76.491 | 16.784 | 1.00 | 14.84 | C |
| ATOM | 1331 | C | LEU | A | 199 | 113.491 | 75.453 | 17.345 | 1.00 | 11.35 | C |
| ATOM | 1332 | O | LEU | A | 199 | 113.736 | 75.002 | 18.466 | 1.00 | 12.93 | O |
| ATOM | 1333 | N | VAL | A | 200 | 112.974 | 74.734 | 16.365 | 1.00 | 12.93 | N |
| ATOM | 1334 | CA | VAL | A | 200 | 112.605 | 73.340 | 16.490 | 1.00 | 12.79 | C |
| ATOM | 1335 | CB | VAL | A | 200 | 111.226 | 73.062 | 15.920 | 1.00 | 12.17 | C |
| ATOM | 1336 | CG1 | VAL | A | 200 | 110.812 | 71.605 | 16.276 | 1.00 | 14.35 | C |
| ATOM | 1337 | CG2 | VAL | A | 200 | 110.188 | 74.071 | 16.403 | 1.00 | 12.12 | C |
| ATOM | 1338 | C | VAL | A | 200 | 113.588 | 72.464 | 15.720 | 1.00 | 14.21 | C |
| ATOM | 1339 | O | VAL | A | 200 | 113.793 | 72.626 | 14.501 | 1.00 | 15.13 | O |
| ATOM | 1340 | N | CYS | A | 201 | 114.216 | 71.547 | 16.443 | 1.00 | 14.83 | N |
| ATOM | 1341 | CA | CYS | A | 201 | 115.171 | 70.608 | 15.861 | 1.00 | 15.52 | C |
| ATOM | 1342 | CB | CYS | A | 201 | 116.577 | 70.949 | 16.342 | 1.00 | 15.36 | C |
| ATOM | 1343 | SG | CYS | A | 201 | 117.122 | 72.673 | 16.214 | 1.00 | 14.85 | S |
| ATOM | 1344 | C | CYS | A | 201 | 114.798 | 69.206 | 16.348 | 1.00 | 16.45 | C |
| ATOM | 1345 | O | CYS | A | 201 | 114.756 | 68.989 | 17.563 | 1.00 | 16.35 | O |
| ATOM | 1346 | N | ASN | A | 204 | 114.526 | 68.288 | 15.417 | 1.00 | 17.73 | N |
| ATOM | 1347 | CA | ASN | A | 204 | 114.070 | 66.910 | 15.738 | 1.00 | 18.75 | C |
| ATOM | 1348 | CB | ASN | A | 204 | 115.176 | 66.124 | 16.426 | 1.00 | 18.52 | C |
| ATOM | 1349 | CG | ASN | A | 204 | 116.457 | 66.181 | 15.686 | 1.00 | 19.70 | C |
| ATOM | 1350 | OD1 | ASN | A | 204 | 116.473 | 65.929 | 14.490 | 1.00 | 21.71 | O |
| ATOM | 1351 | ND2 | ASN | A | 204 | 117.545 | 66.537 | 16.371 | 1.00 | 22.28 | N |
| ATOM | 1352 | C | ASN | A | 204 | 112.798 | 66.866 | 16.590 | 1.00 | 19.61 | C |
| ATOM | 1353 | O | ASN | A | 204 | 112.647 | 66.021 | 17.482 | 1.00 | 20.27 | O |
| ATOM | 1354 | N | GLY | A | 205 | 111.880 | 67.769 | 16.289 | 1.00 | 19.65 | N |
| ATOM | 1355 | CA | GLY | A | 205 | 110.650 | 67.910 | 17.033 | 1.00 | 19.96 | C |
| ATOM | 1356 | C | GLY | A | 205 | 110.783 | 68.601 | 18.390 | 1.00 | 18.96 | C |
| ATOM | 1357 | O | GLY | A | 205 | 109.764 | 68.779 | 19.054 | 1.00 | 21.26 | O |
| ATOM | 1358 | N | LEU | A | 208 | 111.982 | 69.004 | 18.800 | 1.00 | 17.82 | N |
| ATOM | 1359 | CA | LEU | A | 208 | 112.215 | 69.565 | 20.136 | 1.00 | 17.32 | C |
| ATOM | 1360 | CB | LEU | A | 208 | 113.319 | 68.819 | 20.890 | 1.00 | 17.51 | C |
| ATOM | 1361 | CG | LEU | A | 208 | 112.999 | 67.455 | 21.553 | 1.00 | 21.08 | C |
| ATOM | 1362 | CD1 | LEU | A | 208 | 111.811 | 66.743 | 20.995 | 1.00 | 21.96 | C |
| ATOM | 1363 | CD2 | LEU | A | 208 | 114.231 | 66.578 | 21.608 | 1.00 | 20.74 | C |
| ATOM | 1364 | C | LEU | A | 208 | 112.605 | 71.031 | 20.099 | 1.00 | 15.60 | C |
| ATOM | 1365 | O | LEU | A | 208 | 113.235 | 71.480 | 19.177 | 1.00 | 15.41 | O |
| ATOM | 1366 | N | ILE | A | 209 | 112.258 | 71.774 | 21.136 | 1.00 | 14.77 | N |
| ATOM | 1367 | CA | ILE | A | 209 | 112.621 | 73.209 | 21.152 | 1.00 | 12.39 | C |
| ATOM | 1368 | CB | ILE | A | 209 | 111.606 | 74.047 | 21.932 | 1.00 | 12.65 | C |
| ATOM | 1369 | CG1 | ILE | A | 209 | 110.158 | 73.691 | 21.593 | 1.00 | 12.10 | C |
| ATOM | 1370 | CD1 | ILE | A | 209 | 109.766 | 73.749 | 20.161 | 1.00 | 14.66 | C |
| ATOM | 1371 | CG2 | ILE | A | 209 | 111.858 | 75.542 | 21.676 | 1.00 | 11.82 | C |
| ATOM | 1372 | C | ILE | A | 209 | 114.003 | 73.387 | 21.716 | 1.00 | 11.78 | C |
| ATOM | 1373 | O | ILE | A | 209 | 114.210 | 73.309 | 22.927 | 1.00 | 12.58 | O |
| ATOM | 1374 | N | HIS | A | 210 | 114.975 | 73.659 | 20.843 | 1.00 | 11.89 | N |
| ATOM | 1375 | CA | HIS | A | 210 | 116.358 | 73.812 | 21.274 | 1.00 | 12.21 | C |
| ATOM | 1376 | CB | HIS | A | 210 | 117.283 | 73.055 | 20.317 | 1.00 | 12.73 | C |
| ATOM | 1377 | CG | HIS | A | 210 | 117.215 | 71.559 | 20.446 | 1.00 | 13.21 | C |
| ATOM | 1378 | ND1 | HIS | A | 210 | 118.223 | 70.823 | 21.021 | 1.00 | 16.36 | N |
| ATOM | 1379 | CE1 | HIS | A | 210 | 117.905 | 69.545 | 21.004 | 1.00 | 15.65 | C |
| ATOM | 1380 | NE2 | HIS | A | 210 | 116.731 | 69.418 | 20.414 | 1.00 | 16.62 | N |
| ATOM | 1381 | CD2 | HIS | A | 210 | 116.273 | 70.666 | 20.065 | 1.00 | 17.80 | C |
| ATOM | 1382 | C | HIS | A | 210 | 116.763 | 75.260 | 21.356 | 1.00 | 11.27 | C |
| ATOM | 1383 | O | HIS | A | 210 | 117.802 | 75.558 | 21.912 | 1.00 | 12.16 | O |
| ATOM | 1384 | N | GLY | A | 211 | 115.956 | 76.174 | 20.791 | 1.00 | 10.33 | N |
| ATOM | 1385 | CA | GLY | A | 211 | 116.268 | 77.581 | 20.850 | 1.00 | 9.45 | C |
| ATOM | 1386 | C | GLY | A | 211 | 115.070 | 78.503 | 20.995 | 1.00 | 8.37 | C |
| ATOM | 1387 | O | GLY | A | 211 | 113.967 | 78.122 | 20.686 | 1.00 | 8.77 | O |
| ATOM | 1388 | N | ILE | A | 212 | 115.347 | 79.701 | 21.474 | 1.00 | 8.37 | N |
| ATOM | 1389 | CA | ILE | A | 212 | 114.421 | 80.845 | 21.528 | 1.00 | 9.35 | C |
| ATOM | 1390 | CB | ILE | A | 212 | 114.150 | 81.230 | 22.983 | 1.00 | 9.69 | C |
| ATOM | 1391 | CG1 | ILE | A | 212 | 113.491 | 80.065 | 23.736 | 1.00 | 8.63 | C |
| ATOM | 1392 | CD1 | ILE | A | 212 | 113.491 | 80.217 | 25.206 | 1.00 | 9.98 | C |
| ATOM | 1393 | CG2 | ILE | A | 212 | 113.235 | 82.494 | 23.077 | 1.00 | 10.69 | C |
| ATOM | 1394 | C | ILE | A | 212 | 115.130 | 81.997 | 20.794 | 1.00 | 9.43 | C |
| ATOM | 1395 | O | ILE | A | 212 | 116.219 | 82.426 | 21.174 | 1.00 | 10.08 | O |
| ATOM | 1396 | N | ALA | A | 213 | 114.582 | 82.457 | 19.690 | 1.00 | 10.33 | N |
| ATOM | 1397 | CA | ALA | A | 213 | 115.210 | 83.557 | 18.952 | 1.00 | 8.97 | C |
| ATOM | 1398 | CB | ALA | A | 213 | 114.258 | 84.027 | 17.838 | 1.00 | 10.65 | C |
| ATOM | 1399 | C | ALA | A | 213 | 115.603 | 84.736 | 19.815 | 1.00 | 9.80 | C |
| ATOM | 1400 | O | ALA | A | 213 | 114.812 | 85.236 | 20.592 | 1.00 | 9.31 | O |
| ATOM | 1401 | N | SER | A | 214 | 116.838 | 85.209 | 19.644 | 1.00 | 9.31 | N |
| ATOM | 1402 | CA | SER | A | 214 | 117.394 | 86.188 | 20.534 | 1.00 | 9.73 | C |
| ATOM | 1403 | CB | SER | A | 214 | 118.538 | 85.591 | 21.365 | 1.00 | 8.22 | C |
| ATOM | 1404 | OG | SER | A | 214 | 118.989 | 86.548 | 22.318 | 1.00 | 9.20 | O |
| ATOM | 1405 | C | SER | A | 214 | 117.875 | 87.399 | 19.755 | 1.00 | 10.50 | C |
| ATOM | 1406 | O | SER | A | 214 | 117.472 | 88.518 | 20.036 | 1.00 | 11.88 | O |
| ATOM | 1407 | N | PHE | A | 215 | 118.759 | 87.179 | 18.780 | 1.00 | 12.20 | N |
| ATOM | 1408 | CA | PHE | A | 215 | 119.256 | 88.297 | 18.020 | 1.00 | 12.08 | C |
| ATOM | 1409 | CB | PHE | A | 215 | 120.223 | 89.142 | 18.863 | 1.00 | 12.31 | C |
| ATOM | 1410 | CG | PHE | A | 215 | 121.513 | 88.457 | 19.230 | 1.00 | 12.11 | C |
| ATOM | 1411 | CD1 | PHE | A | 215 | 121.641 | 87.783 | 20.433 | 1.00 | 9.79 | C |
| ATOM | 1412 | CE1 | PHE | A | 215 | 122.871 | 87.150 | 20.806 | 1.00 | 11.91 | C |
| ATOM | 1413 | CZ | PHE | A | 215 | 123.982 | 87.275 | 19.934 | 1.00 | 14.83 | C |
| ATOM | 1414 | CE2 | PHE | A | 215 | 123.860 | 87.994 | 18.753 | 1.00 | 14.16 | C |
| ATOM | 1415 | CD2 | PHE | A | 215 | 122.651 | 88.602 | 18.408 | 1.00 | 12.73 | C |
| ATOM | 1416 | C | PHE | A | 215 | 119.883 | 87.971 | 16.672 | 1.00 | 11.70 | C |
| ATOM | 1417 | O | PHE | A | 215 | 120.363 | 86.865 | 16.448 | 1.00 | 11.77 | O |
| ATOM | 1418 | N | VAL | A | 216 | 119.917 | 89.002 | 15.812 | 1.00 | 11.46 | N |
| ATOM | 1419 | CA | VAL | A | 216 | 120.628 | 88.961 | 14.560 | 1.00 | 12.52 | C |
| ATOM | 1420 | CB | VAL | A | 216 | 119.743 | 89.359 | 13.362 | 1.00 | 12.94 | C |
| ATOM | 1421 | CG1 | VAL | A | 216 | 118.519 | 88.489 | 13.305 | 1.00 | 13.64 | C |
| ATOM | 1422 | CG2 | VAL | A | 216 | 119.398 | 90.869 | 13.369 | 1.00 | 14.39 | C |
| ATOM | 1423 | C | VAL | A | 216 | 121.877 | 89.877 | 14.621 | 1.00 | 13.25 | C |
| ATOM | 1424 | O | VAL | A | 216 | 122.028 | 90.716 | 15.543 | 1.00 | 13.11 | O |
| ATOM | 1425 | N | ARG | A | 217 | 122.772 | 89.680 | 13.657 | 1.00 | 14.31 | N |
| ATOM | 1426 | CA | ARG | A | 217 | 124.004 | 90.503 | 13.538 | 1.00 | 15.51 | C |
| ATOM | 1427 | CB | ARG | A | 217 | 125.263 | 89.736 | 13.990 | 1.00 | 16.17 | C |
| ATOM | 1428 | CG | ARG | A | 217 | 125.467 | 89.562 | 15.486 | 1.00 | 21.52 | C |
| ATOM | 1429 | CD | ARG | A | 217 | 126.885 | 88.995 | 15.870 | 1.00 | 26.60 | C |
| ATOM | 1430 | NE | ARG | A | 217 | 128.009 | 89.813 | 15.371 | 1.00 | 31.15 | N |
| ATOM | 1431 | CZ | ARG | A | 217 | 129.318 | 89.466 | 15.452 | 1.00 | 33.47 | C |
| ATOM | 1432 | NH1 | ARG | A | 217 | 129.678 | 88.302 | 15.984 | 1.00 | 32.62 | N |
| ATOM | 1433 | NH2 | ARG | A | 217 | 130.264 | 90.293 | 14.998 | 1.00 | 35.81 | N |
| ATOM | 1434 | C | ARG | A | 217 | 124.161 | 90.836 | 12.077 | 1.00 | 14.86 | C |
| ATOM | 1435 | O | ARG | A | 217 | 123.936 | 89.963 | 11.208 | 1.00 | 16.14 | O |
| ATOM | 1436 | N | GLY | A | 218 | 124.581 | 92.073 | 11.813 | 1.00 | 15.65 | N |
| ATOM | 1437 | CA | GLY | A | 218 | 124.671 | 92.622 | 10.471 | 1.00 | 16.59 | C |
| ATOM | 1438 | C | GLY | A | 218 | 123.317 | 92.835 | 9.773 | 1.00 | 16.73 | C |
| ATOM | 1439 | O | GLY | A | 218 | 123.223 | 92.719 | 8.542 | 1.00 | 15.85 | O |
| ATOM | 1440 | N | GLY | A | 219 | 122.278 | 93.126 | 10.569 | 1.00 | 15.73 | N |
| ATOM | 1441 | CA | GLY | A | 219 | 120.902 | 93.159 | 10.073 | 1.00 | 15.73 | C |
| ATOM | 1442 | C | GLY | A | 219 | 120.359 | 91.765 | 9.856 | 1.00 | 14.55 | C |
| ATOM | 1443 | O | GLY | A | 219 | 121.072 | 90.769 | 9.992 | 1.00 | 14.00 | O |
| ATOM | 1444 | N | CYS | A | 220 | 119.084 | 91.681 | 9.465 | 1.00 | 13.62 | N |
| ATOM | 1445 | CA | CYS | A | 220 | 118.460 | 90.405 | 9.185 | 1.00 | 12.09 | C |
| ATOM | 1446 | CB | CYS | A | 220 | 116.919 | 90.520 | 9.190 | 1.00 | 12.03 | C |
| ATOM | 1447 | SG | CYS | A | 220 | 116.324 | 91.457 | 10.591 | 1.00 | 11.25 | S |
| ATOM | 1448 | C | CYS | A | 220 | 118.930 | 89.862 | 7.860 | 1.00 | 12.70 | C |
| ATOM | 1449 | O | CYS | A | 220 | 119.223 | 90.644 | 6.935 | 1.00 | 13.78 | O |
| ATOM | 1450 | N | ALA | A | 221 | 119.081 | 88.531 | 7.778 | 1.00 | 12.20 | N |
| ATOM | 1451 | CA | ALA | A | 221 | 119.379 | 87.851 | 6.524 | 1.00 | 11.80 | C |
| ATOM | 1452 | CB | ALA | A | 221 | 118.138 | 87.881 | 5.611 | 1.00 | 11.81 | C |
| ATOM | 1453 | C | ALA | A | 221 | 120.572 | 88.533 | 5.840 | 1.00 | 13.24 | C |
| ATOM | 1454 | O | ALA | A | 221 | 120.528 | 88.858 | 4.638 | 1.00 | 14.09 | O |
| ATOM | 1455 | N | SER | A | 222 | 121.604 | 88.809 | 6.631 | 1.00 | 13.15 | N |
| ATOM | 1456 | CA | SER | A | 222 | 122.902 | 89.308 | 6.128 | 1.00 | 13.05 | C |
| ATOM | 1457 | CB | SER | A | 222 | 123.841 | 89.546 | 7.319 | 1.00 | 11.71 | C |
| ATOM | 1458 | OG | SER | A | 222 | 124.192 | 88.339 | 7.967 | 1.00 | 12.18 | O |
| ATOM | 1459 | C | SER | A | 222 | 123.605 | 88.399 | 5.128 | 1.00 | 13.33 | C |
| ATOM | 1460 | O | SER | A | 222 | 124.278 | 88.869 | 4.204 | 1.00 | 15.05 | O |
| ATOM | 1461 | N | GLY | A | 222A | 123.480 | 87.099 | 5.341 | 1.00 | 13.24 | N |
| ATOM | 1462 | CA | GLY | A | 222A | 124.238 | 86.096 | 4.637 | 1.00 | 13.56 | C |
| ATOM | 1463 | C | GLY | A | 222A | 125.697 | 85.996 | 5.132 | 1.00 | 14.02 | C |
| ATOM | 1464 | O | GLY | A | 222A | 126.476 | 85.283 | 4.531 | 1.00 | 14.50 | O |
| ATOM | 1465 | N | LEU | A | 223 | 126.035 | 86.744 | 6.183 | 1.00 | 14.48 | N |
| ATOM | 1466 | CA | LEU | A | 223 | 127.377 | 86.916 | 6.727 | 1.00 | 14.70 | C |
| ATOM | 1467 | CB | LEU | A | 223 | 127.753 | 88.403 | 6.711 | 1.00 | 14.48 | C |
| ATOM | 1468 | CG | LEU | A | 223 | 127.925 | 89.039 | 5.313 | 1.00 | 16.12 | C |
| ATOM | 1469 | CD1 | LEU | A | 223 | 128.287 | 90.444 | 5.440 | 1.00 | 16.78 | C |
| ATOM | 1470 | CD2 | LEU | A | 223 | 128.996 | 88.299 | 4.498 | 1.00 | 18.60 | C |
| ATOM | 1471 | C | LEU | A | 223 | 127.474 | 86.361 | 8.151 | 1.00 | 14.81 | C |
| ATOM | 1472 | O | LEU | A | 223 | 128.476 | 85.757 | 8.518 | 1.00 | 15.31 | O |
| ATOM | 1473 | N | TYR | A | 224 | 126.430 | 86.577 | 8.958 | 1.00 | 14.98 | N |
| ATOM | 1474 | CA | TYR | A | 224 | 126.399 | 86.161 | 10.358 | 1.00 | 14.84 | C |
| ATOM | 1475 | CB | TYR | A | 224 | 126.264 | 87.350 | 11.295 | 1.00 | 14.87 | C |
| ATOM | 1476 | CG | TYR | A | 224 | 127.278 | 88.417 | 11.139 | 1.00 | 15.90 | C |
| ATOM | 1477 | CD1 | TYR | A | 224 | 128.496 | 88.348 | 11.810 | 1.00 | 17.42 | C |
| ATOM | 1478 | CE1 | TYR | A | 224 | 129.452 | 89.354 | 11.666 | 1.00 | 18.56 | C |
| ATOM | 1479 | CZ | TYR | A | 224 | 129.177 | 90.446 | 10.896 | 1.00 | 17.82 | C |
| ATOM | 1480 | OH | TYR | A | 224 | 130.119 | 91.439 | 10.775 | 1.00 | 18.92 | O |
| ATOM | 1481 | CE2 | TYR | A | 224 | 127.975 | 90.552 | 10.231 | 1.00 | 16.36 | C |
| ATOM | 1492 | CD2 | TYR | A | 224 | 127.024 | 89.516 | 10.356 | 1.00 | 16.28 | C |
| ATOM | 1483 | C | TYR | A | 224 | 125.196 | 85.297 | 10.622 | 1.00 | 14.79 | C |
| ATOM | 1484 | O | TYR | A | 224 | 124.089 | 85.557 | 10.089 | 1.00 | 14.68 | O |
| ATOM | 1485 | N | PRO | A | 225 | 125.387 | 84.261 | 11.434 | 1.00 | 15.29 | N |
| ATOM | 1486 | CA | PRO | A | 225 | 124.280 | 83.372 | 11.770 | 1.00 | 13.67 | C |
| ATOM | 1487 | CB | PRO | A | 225 | 124.973 | 82.169 | 12.405 | 1.00 | 13.11 | C |
| ATOM | 1488 | CG | PRO | A | 225 | 126.177 | 82.753 | 13.054 | 1.00 | 15.98 | C |
| ATOM | 1489 | CD | PRO | A | 225 | 126.618 | 83.881 | 12.150 | 1.00 | 15.79 | C |
| ATOM | 1490 | C | PRO | A | 225 | 123.355 | 84.062 | 12.760 | 1.00 | 12.28 | C |
| ATOM | 1491 | O | PRO | A | 225 | 123.730 | 85.049 | 13.381 | 1.00 | 14.14 | O |
| ATOM | 1492 | N | ASP | A | 226 | 122.139 | 83.554 | 12.873 | 1.00 | 11.23 | N |
| ATOM | 1493 | CA | ASP | A | 226 | 121.214 | 84.039 | 13.876 | 1.00 | 10.62 | C |
| ATOM | 1494 | CB | ASP | A | 226 | 119.800 | 83.740 | 13.459 | 1.00 | 10.07 | C |
| ATOM | 1495 | CG | ASP | A | 226 | 119.421 | 84.447 | 12.149 | 1.00 | 9.72 | C |
| ATOM | 1496 | OD1 | ASP | A | 226 | 118.493 | 83.964 | 11.492 | 1.00 | 8.16 | O |
| ATOM | 1497 | OD2 | ASP | A | 226 | 119.977 | 85.528 | 11.787 | 1.00 | 11.34 | O |
| ATOM | 1498 | C | ASP | A | 226 | 121.468 | 83.349 | 15.223 | 1.00 | 10.31 | C |
| ATOM | 1499 | O | ASP | A | 226 | 121.845 | 82.226 | 15.257 | 1.00 | 13.37 | O |
| ATOM | 1500 | N | ALA | A | 227 | 121.177 | 84.039 | 16.311 | 1.00 | 11.09 | N |
| ATOM | 1501 | CA | ALA | A | 227 | 121.467 | 83.551 | 17.657 | 1.00 | 10.47 | C |
| ATOM | 1502 | CB | ALA | A | 227 | 122.335 | 84.559 | 18.385 | 1.00 | 11.26 | C |
| ATOM | 1503 | C | ALA | A | 227 | 120.204 | 83.312 | 18.440 | 1.00 | 10.60 | C |
| ATOM | 1504 | O | ALA | A | 227 | 119.300 | 84.144 | 18.430 | 1.00 | 10.11 | O |
| ATOM | 1505 | N | PHE | A | 228 | 120.170 | 82.157 | 19.099 | 1.00 | 11.32 | N |
| ATOM | 1506 | CA | PHE | A | 228 | 119.038 | 81.669 | 19.882 | 1.00 | 10.10 | C |
| ATOM | 1507 | CB | PHE | A | 228 | 118.524 | 80.384 | 19.239 | 1.00 | 9.83 | C |
| ATOM | 1508 | CG | PHE | A | 228 | 117.841 | 80.588 | 17.913 | 1.00 | 10.43 | C |
| ATOM | 1509 | CD1 | PHE | A | 228 | 116.458 | 80.668 | 17.825 | 1.00 | 9.30 | C |
| ATOM | 1510 | CE1 | PHE | A | 228 | 115.815 | 80.829 | 16.590 | 1.00 | 11.17 | C |
| ATOM | 1511 | CZ | PHE | A | 228 | 116.582 | 80.942 | 15.421 | 1.00 | 10.11 | C |
| ATOM | 1512 | CE2 | PHE | A | 228 | 117.976 | 80.847 | 15.507 | 1.00 | 9.61 | C |
| ATOM | 1513 | CD2 | PHE | A | 228 | 118.595 | 80.684 | 16.722 | 1.00 | 9.90 | C |
| ATOM | 1514 | C | PHE | A | 228 | 119.505 | 81.429 | 21.309 | 1.00 | 10.91 | C |
| ATOM | 1515 | O | PHE | A | 228 | 120.665 | 81.000 | 21.524 | 1.00 | 11.00 | O |
| ATOM | 1516 | N | ALA | A | 229 | 118.670 | 81.734 | 22.307 | 1.00 | 10.14 | N |
| ATOM | 1517 | CA | ALA | A | 229 | 118.944 | 81.264 | 23.664 | 1.00 | 10.04 | C |
| ATOM | 1518 | CB | ALA | A | 229 | 117.914 | 81.761 | 24.639 | 1.00 | 8.92 | C |
| ATOM | 1519 | C | ALA | A | 229 | 118.944 | 79.753 | 23.669 | 1.00 | 10.20 | C |
| ATOM | 1520 | O | ALA | A | 229 | 118.070 | 79.151 | 23.076 | 1.00 | 8.82 | O |
| ATOM | 1521 | N | PRO | A | 230 | 119.940 | 79.147 | 24.319 | 1.00 | 10.55 | N |
| ATOM | 1522 | CA | PRO | A | 230 | 120.120 | 77.702 | 24.285 | 1.00 | 11.56 | C |
| ATOM | 1523 | CB | PRO | A | 230 | 121.616 | 77.535 | 24.643 | 1.00 | 11.12 | C |
| ATOM | 1524 | CG | PRO | A | 230 | 121.870 | 78.634 | 25.567 | 1.00 | 11.47 | C |
| ATOM | 1525 | CD | PRO | A | 230 | 121.029 | 79.838 | 25.049 | 1.00 | 9.84 | C |
| ATOM | 1526 | C | PRO | A | 230 | 119.239 | 77.006 | 25.311 | 1.00 | 12.10 | C |
| ATOM | 1527 | O | PRO | A | 230 | 119.624 | 76.954 | 26.472 | 1.00 | 12.46 | O |
| ATOM | 1528 | N | VAL | A | 231 | 118.073 | 76.548 | 24.887 | 1.00 | 12.37 | N |
| ATOM | 1529 | CA | VAL | A | 231 | 117.072 | 75.997 | 25.801 | 1.00 | 12.53 | C |
| ATOM | 1530 | CB | VAL | A | 231 | 115.821 | 75.530 | 25.025 | 1.00 | 12.67 | C |
| ATOM | 1531 | CG1 | VAL | A | 231 | 115.114 | 76.734 | 24.310 | 1.00 | 11.72 | C |
| ATOM | 1532 | CG2 | VAL | A | 231 | 114.832 | 74.838 | 25.972 | 1.00 | 12.34 | C |
| ATOM | 1533 | C | VAL | A | 231 | 117.633 | 74.832 | 26.643 | 1.00 | 13.15 | C |
| ATOM | 1534 | O | VAL | A | 231 | 117.410 | 74.778 | 27.846 | 1.00 | 12.40 | O |
| ATOM | 1535 | N | ALA | A | 232 | 118.390 | 73.943 | 26.008 | 1.00 | 12.71 | N |
| ATOM | 1536 | CA | ALA | A | 232 | 118.963 | 72.768 | 26.689 | 1.00 | 14.18 | C |
| ATOM | 1537 | CB | ALA | A | 232 | 119.785 | 71.959 | 25.704 | 1.00 | 14.09 | C |
| ATOM | 1538 | C | ALA | A | 232 | 119.805 | 73.077 | 27.923 | 1.00 | 15.19 | C |
| ATOM | 1539 | O | ALA | A | 232 | 119.994 | 72.192 | 28.790 | 1.00 | 15.41 | O |
| ATOM | 1540 | N | GLN | A | 233 | 120.308 | 74.299 | 28.012 | 1.00 | 14.26 | N |
| ATOM | 1541 | CA | GLN | A | 233 | 121.147 | 74.737 | 29.127 | 1.00 | 15.88 | C |
| ATOM | 1542 | CB | GLN | A | 233 | 122.066 | 75.893 | 28.704 | 1.00 | 15.80 | C |
| ATOM | 1543 | CG | GLN | A | 233 | 123.086 | 75.564 | 27.576 | 1.00 | 19.53 | C |
| ATOM | 1544 | CD | GLN | A | 233 | 124.130 | 76.687 | 27.274 | 1.00 | 23.97 | C |
| ATOM | 1545 | OE1 | GLN | A | 233 | 124.339 | 77.630 | 28.074 | 1.00 | 26.97 | O |
| ATOM | 1546 | NE2 | GLN | A | 233 | 124.779 | 76.580 | 26.097 | 1.00 | 26.40 | N |
| ATOM | 1547 | C | GLN | A | 233 | 120.257 | 75.159 | 30.318 | 1.00 | 15.47 | C |
| ATOM | 1548 | O | GLN | A | 233 | 120.719 | 75.269 | 31.424 | 1.00 | 16.25 | O |
| ATOM | 1549 | N | PHE | A | 234 | 118.978 | 75.385 | 30.051 | 1.00 | 14.85 | N |
| ATOM | 1550 | CA | PHE | A | 234 | 118.007 | 75.770 | 31.061 | 1.00 | 14.16 | C |
| ATOM | 1551 | CB | PHE | A | 234 | 117.278 | 77.008 | 30.531 | 1.00 | 13.21 | C |
| ATOM | 1552 | CG | PHE | A | 234 | 118.183 | 78.182 | 30.403 | 1.00 | 14.38 | C |
| ATOM | 1553 | CD1 | PHE | A | 234 | 118.924 | 78.376 | 29.256 | 1.00 | 12.04 | C |
| ATOM | 1554 | CE1 | PHE | A | 234 | 119.832 | 79.486 | 29.149 | 1.00 | 14.58 | C |
| ATOM | 1555 | CZ | PHE | A | 234 | 119.999 | 80.319 | 30.200 | 1.00 | 13.23 | C |
| ATOM | 1556 | CE2 | PHE | A | 234 | 119.250 | 80.134 | 31.388 | 1.00 | 18.81 | C |
| ATOM | 1557 | CD2 | PHE | A | 234 | 118.368 | 79.041 | 31.490 | 1.00 | 16.41 | C |
| ATOM | 1558 | C | PHE | A | 234 | 116.958 | 74.744 | 31.466 | 1.00 | 14.28 | C |
| ATOM | 1559 | O | PHE | A | 234 | 115.976 | 75.115 | 32.103 | 1.00 | 14.73 | O |
| ATOM | 1560 | N | VAL | A | 235 | 117.111 | 73.491 | 31.070 | 1.00 | 13.87 | N |
| ATOM | 1561 | CA | VAL | A | 235 | 116.053 | 72.502 | 31.270 | 1.00 | 14.29 | C |
| ATOM | 1562 | CB | VAL | A | 235 | 116.286 | 71.256 | 30.399 | 1.00 | 14.98 | C |
| ATOM | 1563 | CG1 | VAL | A | 235 | 115.278 | 70.140 | 30.716 | 1.00 | 14.35 | C |
| ATOM | 1564 | CG2 | VAL | A | 235 | 116.188 | 71.615 | 28.953 | 1.00 | 15.74 | C |
| ATOM | 1565 | C | VAL | A | 235 | 115.815 | 72.131 | 32.764 | 1.00 | 14.72 | C |
| ATOM | 1566 | O | VAL | A | 235 | 114.658 | 72.033 | 33.189 | 1.00 | 13.41 | O |
| ATOM | 1567 | N | ASN | A | 236 | 116.865 | 71.977 | 33.567 | 1.00 | 14.24 | N |
| ATOM | 1568 | CA | ASN | A | 236 | 116.665 | 71.701 | 34.993 | 1.00 | 14.15 | C |
| ATOM | 1569 | CB | ASN | A | 236 | 117.983 | 71.525 | 35.732 | 1.00 | 14.25 | C |
| ATOM | 1570 | CG | ASN | A | 236 | 118.723 | 70.233 | 35.398 | 1.00 | 17.69 | C |
| ATOM | 1571 | OD1 | ASN | A | 236 | 119.957 | 70.174 | 35.597 | 1.00 | 24.22 | O |
| ATOM | 1572 | ND2 | ASN | A | 236 | 118.015 | 69.198 | 34.926 | 1.00 | 14.15 | N |
| ATOM | 1573 | C | ASN | A | 236 | 115.887 | 72.879 | 35.643 | 1.00 | 14.07 | C |
| ATOM | 1574 | O | ASN | A | 236 | 114.973 | 72.678 | 36.451 | 1.00 | 13.84 | O |
| ATOM | 1575 | N | TRP | A | 237 | 116.264 | 74.101 | 35.276 | 1.00 | 13.94 | N |
| ATOM | 1576 | CA | TRP | A | 237 | 115.615 | 75.320 | 35.767 | 1.00 | 13.19 | C |
| ATOM | 1577 | CB | TRP | A | 237 | 116.337 | 76.540 | 35.213 | 1.00 | 12.88 | C |
| ATOM | 1578 | CG | TRP | A | 237 | 115.770 | 77.893 | 35.595 | 1.00 | 12.50 | C |
| ATOM | 1579 | CD1 | TRP | A | 237 | 115.743 | 78.467 | 36.835 | 1.00 | 13.27 | C |
| ATOM | 1580 | NE1 | TRP | A | 237 | 115.191 | 79.726 | 36.759 | 1.00 | 13.22 | N |
| ATOM | 1581 | CE2 | TRP | A | 237 | 114.897 | 80.018 | 35.456 | 1.00 | 13.08 | C |
| ATOM | 1582 | CD2 | TRP | A | 237 | 115.260 | 78.893 | 34.687 | 1.00 | 13.13 | C |
| ATOM | 1583 | CE3 | TRP | A | 237 | 115.010 | 78.915 | 33.315 | 1.00 | 13.02 | C |
| ATOM | 1584 | CZ3 | TRP | A | 237 | 114.468 | 80.055 | 32.745 | 1.00 | 13.63 | C |
| ATOM | 1585 | CH2 | TRP | A | 237 | 114.133 | 81.172 | 33.532 | 1.00 | 13.63 | C |
| ATOM | 1586 | CZ2 | TRP | A | 237 | 114.331 | 81.174 | 34.883 | 1.00 | 13.73 | C |
| ATOM | 1587 | C | TRP | A | 237 | 114.138 | 75.359 | 35.333 | 1.00 | 12.57 | C |
| ATOM | 1588 | O | TRP | A | 237 | 113.260 | 75.620 | 36.158 | 1.00 | 13.53 | O |
| ATOM | 1589 | N | ILE | A | 238 | 113.879 | 75.087 | 34.063 | 1.00 | 11.73 | N |
| ATOM | 1590 | CA | ILE | A | 238 | 112.514 | 74.962 | 33.541 | 1.00 | 12.26 | C |
| ATOM | 1591 | CB | ILE | A | 238 | 112.501 | 74.683 | 32.019 | 1.00 | 12.06 | C |
| ATOM | 1592 | CG1 | ILE | A | 238 | 112.999 | 75.936 | 31.272 | 1.00 | 12.31 | C |
| ATOM | 1593 | CD1 | ILE | A | 238 | 113.327 | 75.729 | 29.799 | 1.00 | 14.40 | C |
| ATOM | 1594 | CG2 | ILE | A | 238 | 111.106 | 74.356 | 31.552 | 1.00 | 13.55 | C |
| ATOM | 1595 | C | ILE | A | 238 | 111.699 | 73.932 | 34.307 | 1.00 | 12.08 | C |
| ATOM | 1596 | O | ILE | A | 238 | 110.656 | 74.247 | 34.819 | 1.00 | 11.36 | O |
| ATOM | 1597 | N | ASP | A | 239 | 112.221 | 72.714 | 34.418 | 1.00 | 12.98 | N |
| ATOM | 1598 | CA | ASP | A | 239 | 111.596 | 71.655 | 35.199 | 1.00 | 13.35 | C |
| ATOM | 1599 | CB | ASP | A | 239 | 112.578 | 70.477 | 35.342 | 1.00 | 13.18 | C |
| ATOM | 1600 | CG | ASP | A | 239 | 112.744 | 69.663 | 34.064 | 1.00 | 16.12 | C |
| ATOM | 1601 | OD1 | ASP | A | 239 | 113.546 | 68.662 | 34.078 | 1.00 | 14.96 | O |
| ATOM | 1602 | OD2 | ASP | A | 239 | 112.132 | 69.924 | 33.013 | 1.00 | 14.55 | O |
| ATOM | 1603 | C | ASP | A | 239 | 111.219 | 72.109 | 36.612 | 1.00 | 14.05 | C |
| ATOM | 1604 | O | ASP | A | 239 | 110.198 | 71.684 | 37.139 | 1.00 | 14.23 | O |
| ATOM | 1605 | N | SER | A | 240 | 112.061 | 72.946 | 37.223 | 1.00 | 13.21 | N |
| ATOM | 1606 | CA | SER | A | 240 | 111.882 | 73.385 | 38.619 | 1.00 | 13.39 | C |
| ATOM | 1607 | CB | SER | A | 240 | 113.148 | 74.034 | 39.185 | 1.00 | 12.69 | C |
| ATOM | 1608 | OG | SER | A | 240 | 113.396 | 75.365 | 38.672 | 1.00 | 15.12 | O |
| ATOM | 1609 | C | SER | A | 240 | 110.718 | 74.373 | 38.744 | 1.00 | 13.54 | C |
| ATOM | 1610 | O | SER | A | 240 | 110.176 | 74.577 | 39.833 | 1.00 | 13.80 | O |
| ATOM | 1611 | N | ILE | A | 241 | 110.347 | 74.979 | 37.625 | 1.00 | 13.32 | N |
| ATOM | 1612 | CA | ILE | A | 241 | 109.290 | 76.004 | 37.581 | 1.00 | 13.17 | C |
| ATOM | 1613 | CB | ILE | A | 241 | 109.806 | 77.213 | 36.722 | 1.00 | 12.44 | C |
| ATOM | 1614 | CG1 | ILE | A | 241 | 110.920 | 77.947 | 37.483 | 1.00 | 13.50 | C |
| ATOM | 1615 | CD1 | ILE | A | 241 | 111.826 | 78.726 | 36.597 | 1.00 | 17.17 | C |
| ATOM | 1616 | CG2 | ILE | A | 241 | 108.725 | 78.263 | 36.454 | 1.00 | 12.59 | C |
| ATOM | 1617 | C | ILE | A | 241 | 107.942 | 75.480 | 37.062 | 1.00 | 14.19 | C |
| ATOM | 1618 | O | ILE | A | 241 | 106.895 | 75.784 | 37.663 | 1.00 | 14.03 | O |
| ATOM | 1619 | N | ILE | A | 242 | 107.935 | 74.718 | 35.967 | 1.00 | 14.30 | N |
| ATOM | 1620 | CA | ILE | A | 242 | 106.679 | 74.515 | 35.233 | 1.00 | 16.20 | C |
| ATOM | 1621 | CB | ILE | A | 242 | 106.887 | 74.359 | 33.709 | 1.00 | 15.17 | C |
| ATOM | 1622 | CG1 | ILE | A | 242 | 107.623 | 73.049 | 33.391 | 1.00 | 15.32 | C |
| ATOM | 1623 | CD1 | ILE | A | 242 | 107.771 | 72.774 | 31.945 | 1.00 | 16.10 | C |
| ATOM | 1624 | CG2 | ILE | A | 242 | 107.623 | 75.556 | 33.159 | 1.00 | 14.29 | C |
| ATOM | 1625 | C | ILE | A | 242 | 105.883 | 73.350 | 35.781 | 1.00 | 19.04 | C |
| ATOM | 1626 | O | ILE | A | 242 | 104.838 | 73.031 | 35.249 | 1.00 | 19.63 | O |
| ATOM | 1627 | N | GLN | A | 243 | 106.433 | 72.716 | 36.813 | 1.00 | 21.69 | N |
| ATOM | 1628 | CA | GLN | A | 243 | 105.763 | 71.673 | 37.567 | 1.00 | 24.53 | C |
| ATOM | 1629 | CB | GLN | A | 243 | 106.636 | 70.446 | 37.637 | 1.00 | 24.30 | C |
| ATOM | 1630 | CG | GLN | A | 243 | 107.118 | 69.941 | 36.333 | 1.00 | 26.26 | C |
| ATOM | 1631 | CD | GLN | A | 243 | 107.935 | 68.672 | 36.522 | 1.00 | 30.24 | C |
| ATOM | 1632 | OE1 | GLN | A | 243 | 109.173 | 68.712 | 36.792 | 1.00 | 32.64 | O |
| ATOM | 1633 | NE2 | GLN | A | 243 | 107.267 | 67.548 | 36.394 | 1.00 | 28.99 | N |
| ATOM | 1634 | C | GLN | A | 243 | 105.526 | 72.189 | 38.986 | 1.00 | 26.38 | C |
| ATOM | 1635 | O | GLN | A | 243 | 104.389 | 72.454 | 39.330 | 1.00 | 28.14 | O |
| ATOM | 1637 | N | ILE | B | 16 | 110.084 | 107.821 | 52.991 | 1.00 | 11.47 | N |
| ATOM | 1638 | CA | ILE | B | 16 | 109.762 | 107.488 | 54.424 | 1.00 | 13.18 | C |
| ATOM | 1639 | CB | ILE | B | 16 | 111.052 | 107.246 | 55.267 | 1.00 | 13.19 | C |
| ATOM | 1640 | CG1 | ILE | B | 16 | 111.887 | 106.083 | 54.715 | 1.00 | 12.91 | C |
| ATOM | 1641 | CD1 | ILE | B | 16 | 111.258 | 104.684 | 54.769 | 1.00 | 13.11 | C |
| ATOM | 1642 | CG2 | ILE | B | 16 | 110.701 | 107.060 | 56.744 | 1.00 | 14.45 | C |
| ATOM | 1643 | C | ILE | B | 16 | 108.995 | 108.641 | 55.061 | 1.00 | 14.63 | C |
| ATOM | 1644 | O | ILE | B | 16 | 109.497 | 109.788 | 55.080 | 1.00 | 15.33 | O |
| ATOM | 1645 | N | VAL | B | 17 | 107.762 | 108.348 | 55.516 | 1.00 | 14.42 | N |
| ATOM | 1646 | CA | VAL | B | 17 | 106.881 | 109.306 | 56.174 | 1.00 | 15.00 | C |
| ATOM | 1647 | CB | VAL | B | 17 | 105.400 | 109.068 | 55.792 | 1.00 | 15.59 | C |
| ATOM | 1648 | CG1 | VAL | B | 17 | 104.460 | 110.125 | 56.478 | 1.00 | 17.17 | C |
| ATOM | 1649 | CG2 | VAL | B | 17 | 105.212 | 109.083 | 54.275 | 1.00 | 16.48 | C |
| ATOM | 1650 | C | VAL | B | 17 | 107.017 | 109.160 | 57.676 | 1.00 | 15.20 | C |
| ATOM | 1651 | O | VAL | B | 17 | 106.814 | 108.062 | 58.244 | 1.00 | 17.23 | O |
| ATOM | 1652 | N | GLY | B | 18 | 107.414 | 110.241 | 58.328 | 1.00 | 15.22 | N |
| ATOM | 1653 | CA | GLY | B | 18 | 107.321 | 110.328 | 59.776 | 1.00 | 15.91 | C |
| ATOM | 1654 | C | GLY | B | 18 | 108.569 | 109.753 | 60.442 | 1.00 | 16.18 | C |
| ATOM | 1655 | O | GLY | B | 18 | 108.528 | 109.440 | 61.613 | 1.00 | 16.66 | O |
| ATOM | 1656 | N | GLY | B | 19 | 109.652 | 109.607 | 59.687 | 1.00 | 15.83 | N |
| ATOM | 1657 | CA | GLY | B | 19 | 110.904 | 109.069 | 60.213 | 1.00 | 16.85 | C |
| ATOM | 1658 | C | GLY | B | 19 | 111.893 | 110.180 | 60.550 | 1.00 | 16.66 | C |
| ATOM | 1659 | O | GLY | B | 19 | 111.503 | 111.337 | 60.782 | 1.00 | 16.40 | O |
| ATOM | 1660 | N | ARG | B | 20 | 113.176 | 109.847 | 60.530 | 1.00 | 16.49 | N |
| ATOM | 1661 | CA | ARG | B | 20 | 114.228 | 110.824 | 60.817 | 1.00 | 17.50 | C |
| ATOM | 1662 | CB | ARG | B | 20 | 114.692 | 110.639 | 62.270 | 1.00 | 17.32 | C |
| ATOM | 1663 | CG | ARG | B | 20 | 115.245 | 109.261 | 62.547 | 1.00 | 18.26 | C |
| ATOM | 1664 | CD | ARG | B | 20 | 115.870 | 109.083 | 63.930 | 1.00 | 20.16 | C |
| ATOM | 1665 | NE | ARG | B | 20 | 116.275 | 107.689 | 64.073 | 1.00 | 18.13 | N |
| ATOM | 1666 | CZ | ARG | B | 20 | 115.446 | 106.701 | 64.411 | 1.00 | 19.13 | C |
| ATOM | 1667 | NH1 | ARG | B | 20 | 114.170 | 106.955 | 64.726 | 1.00 | 22.17 | N |
| ATOM | 1668 | NH2 | ARG | B | 20 | 115.888 | 105.467 | 64.459 | 1.00 | 18.33 | N |
| ATOM | 1669 | C | ARG | B | 20 | 115.389 | 110.662 | 59.867 | 1.00 | 17.38 | C |
| ATOM | 1670 | O | ARG | B | 20 | 115.580 | 109.582 | 59.308 | 1.00 | 16.56 | O |
| ATOM | 1671 | N | ARG | B | 21 | 116.199 | 111.707 | 59.710 | 1.00 | 18.01 | N |
| ATOM | 1672 | CA | ARG | B | 21 | 117.502 | 111.570 | 59.046 | 1.00 | 18.42 | C |
| ATOM | 1673 | CB | ARG | B | 21 | 118.354 | 112.832 | 59.227 | 1.00 | 19.82 | C |
| ATOM | 1674 | CG | ARG | B | 21 | 118.285 | 113.855 | 58.135 | 1.00 | 23.93 | C |
| ATOM | 1675 | CD | ARG | B | 21 | 118.390 | 115.278 | 58.623 | 1.00 | 29.80 | C |
| ATOM | 1676 | NE | ARG | B | 21 | 118.075 | 116.207 | 57.537 | 1.00 | 34.23 | N |
| ATOM | 1677 | CZ | ARG | B | 21 | 118.943 | 117.084 | 56.983 | 1.00 | 39.09 | C |
| ATOM | 1678 | NH1 | ARG | B | 21 | 120.199 | 117.211 | 57.433 | 1.00 | 40.33 | N |
| ATOM | 1679 | NH2 | ARG | B | 21 | 118.538 | 117.857 | 55.963 | 1.00 | 39.02 | N |
| ATOM | 1680 | C | ARG | B | 21 | 118.323 | 110.445 | 59.660 | 1.00 | 17.70 | C |
| ATOM | 1681 | O | ARG | B | 21 | 118.486 | 110.407 | 60.875 | 1.00 | 17.86 | O |
| ATOM | 1682 | N | ALA | B | 22 | 118.841 | 109.555 | 56.827 | 1.00 | 17.13 | N |
| ATOM | 1683 | CA | ALA | B | 22 | 119.908 | 108.646 | 59.244 | 1.00 | 17.77 | C |
| ATOM | 1684 | CB | ALA | B | 22 | 120.150 | 107.595 | 58.179 | 1.00 | 16.91 | C |
| ATOM | 1685 | C | ALA | B | 22 | 121.192 | 109.453 | 59.478 | 1.00 | 18.51 | C |
| ATOM | 1686 | O | ALA | B | 22 | 121.370 | 110.517 | 58.910 | 1.00 | 18.25 | O |
| ATOM | 1687 | N | ARG | B | 23 | 122.078 | 108.924 | 60.323 | 1.00 | 20.27 | N |
| ATOM | 1688 | CA | ARG | B | 23 | 123.427 | 109.453 | 60.441 | 1.00 | 20.92 | C |
| ATOM | 1689 | CB | ARG | B | 23 | 124.126 | 108.867 | 61.662 | 1.00 | 21.26 | C |
| ATOM | 1690 | CG | ARG | B | 23 | 123.419 | 109.203 | 62.970 | 1.00 | 26.08 | C |
| ATOM | 1691 | CD | ARG | B | 23 | 122.947 | 110.668 | 63.071 | 1.00 | 31.52 | C |
| ATOM | 1692 | NE | ARG | B | 23 | 124.050 | 111.627 | 62.885 | 1.00 | 38.25 | N |
| ATOM | 1693 | CZ | ARG | B | 23 | 123.934 | 112.891 | 62.435 | 1.00 | 41.73 | C |
| ATOM | 1694 | NH1 | ARG | B | 23 | 122.753 | 113.408 | 62.081 | 1.00 | 43.43 | N |
| ATOM | 1695 | NH2 | ARG | B | 23 | 125.021 | 113.649 | 62.329 | 1.00 | 41.75 | N |
| ATOM | 1696 | C | ARG | B | 23 | 124.177 | 109.112 | 59.160 | 1.00 | 21.09 | C |
| ATOM | 1697 | O | ARG | B | 23 | 123.922 | 108.059 | 58.561 | 1.00 | 19.87 | O |
| ATOM | 1698 | N | PRO | B | 24 | 125.088 | 109.994 | 58.727 | 1.00 | 20.54 | N |
| ATOM | 1699 | CA | PRO | B | 24 | 125.817 | 109.758 | 57.474 | 1.00 | 20.16 | C |
| ATOM | 1700 | CB | PRO | B | 24 | 126.863 | 110.901 | 57.436 | 1.00 | 20.69 | C |
| ATOM | 1701 | CG | PRO | B | 24 | 126.247 | 111.974 | 58.201 | 1.00 | 21.33 | C |
| ATOM | 1702 | CD | PRO | B | 24 | 125.504 | 111.265 | 59.362 | 1.00 | 21.26 | C |
| ATOM | 1703 | C | PRO | B | 24 | 126.446 | 108.364 | 57.481 | 1.00 | 19.82 | C |
| ATOM | 1704 | O | PRO | B | 24 | 127.087 | 107.960 | 58.451 | 1.00 | 18.31 | O |
| ATOM | 1705 | N | HIS | B | 25 | 126.103 | 107.593 | 56.440 | 1.00 | 19.74 | N |
| ATOM | 1706 | CA | HIS | B | 25 | 126.638 | 106.276 | 56.199 | 1.00 | 20.19 | C |
| ATOM | 1707 | CB | HIS | B | 25 | 128.105 | 106.417 | 55.784 | 1.00 | 21.00 | C |
| ATOM | 1708 | CG | HIS | B | 25 | 128.303 | 107.457 | 54.727 | 1.00 | 22.60 | C |
| ATOM | 1709 | ND1 | HIS | B | 25 | 128.242 | 107.171 | 53.381 | 1.00 | 22.96 | N |
| ATOM | 1710 | CE1 | HIS | B | 25 | 128.419 | 108.283 | 52.695 | 1.00 | 22.99 | C |
| ATOM | 1711 | NE2 | HIS | B | 25 | 128.603 | 109.275 | 53.542 | 1.00 | 22.35 | N |
| ATOM | 1712 | CD2 | HIS | B | 25 | 128.474 | 108.793 | 54.820 | 1.00 | 23.33 | C |
| ATOM | 1713 | C | HIS | B | 25 | 126.442 | 105.271 | 57.346 | 1.00 | 19.21 | C |
| ATOM | 1714 | O | HIS | B | 25 | 127.149 | 104.305 | 57.443 | 1.00 | 19.43 | O |
| ATOM | 1715 | N | ALA | B | 26 | 125.400 | 105.475 | 58.137 | 1.00 | 18.84 | N |
| ATOM | 1716 | CA | ALA | B | 26 | 125.028 | 104.540 | 59.178 | 1.00 | 18.16 | C |
| ATOM | 1717 | CB | ALA | B | 26 | 123.840 | 105.095 | 59.934 | 1.00 | 18.51 | C |
| ATOM | 1718 | C | ALA | B | 26 | 124.690 | 103.136 | 58.656 | 1.00 | 17.14 | C |
| ATOM | 1719 | O | ALA | B | 26 | 124.789 | 102.168 | 59.390 | 1.00 | 15.57 | O |
| ATOM | 1720 | N | TRP | B | 27 | 124.267 | 103.039 | 57.394 | 1.00 | 16.33 | N |
| ATOM | 1721 | CA | TRP | B | 27 | 123.841 | 101.774 | 56.797 | 1.00 | 16.22 | C |
| ATOM | 1722 | CB | TRP | B | 27 | 122.305 | 101.756 | 56.629 | 1.00 | 16.25 | C |
| ATOM | 1723 | CG | TRP | B | 27 | 121.650 | 102.299 | 57.825 | 1.00 | 14.68 | C |
| ATOM | 1724 | CD1 | TRP | B | 27 | 120.986 | 103.467 | 57.921 | 1.00 | 15.86 | C |
| ATOM | 1725 | NE1 | TRP | B | 27 | 120.533 | 103.658 | 59.202 | 1.00 | 15.48 | N |
| ATOM | 1726 | CE2 | TRP | B | 27 | 120.913 | 102.597 | 59.970 | 1.00 | 15.73 | C |
| ATOM | 1727 | CD2 | TRP | B | 27 | 121.622 | 101.716 | 59.132 | 1.00 | 15.56 | C |
| ATOM | 1728 | CE3 | TRP | B | 27 | 122.118 | 100.531 | 59.680 | 1.00 | 13.98 | C |
| ATOM | 1729 | CZ3 | TRP | B | 27 | 121.896 | 100.272 | 61.031 | 1.00 | 16.15 | C |
| ATOM | 1730 | CH2 | TRP | B | 27 | 121.209 | 101.169 | 61.843 | 1.00 | 16.07 | C |
| ATOM | 1731 | CZ2 | TRP | B | 27 | 120.704 | 102.345 | 61.342 | 1.00 | 16.23 | C |
| ATOM | 1732 | C | TRP | B | 27 | 124.573 | 101.586 | 55.455 | 1.00 | 16.32 | C |
| ATOM | 1733 | O | TRP | B | 27 | 124.039 | 101.864 | 54.374 | 1.00 | 15.68 | O |
| ATOM | 1734 | N | PRO | B | 28 | 125.835 | 101.155 | 55.530 | 1.00 | 15.58 | N |
| ATOM | 1735 | CA | PRO | B | 28 | 126.704 | 101.170 | 54.349 | 1.00 | 16.25 | C |
| ATOM | 1736 | CB | PRO | B | 28 | 128.072 | 100.823 | 54.938 | 1.00 | 15.98 | C |
| ATOM | 1737 | CG | PRO | B | 28 | 127.782 | 100.090 | 56.125 | 1.00 | 17.00 | C |
| ATOM | 1738 | CD | PRO | B | 28 | 126.526 | 100.627 | 56.714 | 1.00 | 15.47 | C |
| ATOM | 1739 | C | PRO | B | 28 | 126.292 | 100.194 | 53.252 | 1.00 | 16.17 | C |
| ATOM | 1740 | O | PRO | B | 28 | 126.879 | 100.245 | 52.158 | 1.00 | 17.28 | O |
| ATOM | 1741 | N | PHE | B | 29 | 125.298 | 99.347 | 53.535 | 1.00 | 16.40 | N |
| ATOM | 1742 | CA | PHE | B | 29 | 124.709 | 98.419 | 52.578 | 1.00 | 15.58 | C |
| ATOM | 1743 | CB | PHE | B | 29 | 124.210 | 97.167 | 53.294 | 1.00 | 16.24 | C |
| ATOM | 1744 | CG | PHE | B | 29 | 123.328 | 97.445 | 54.475 | 1.00 | 15.55 | C |
| ATOM | 1745 | CD1 | PHE | B | 29 | 121.993 | 97.833 | 54.303 | 1.00 | 12.14 | C |
| ATOM | 1746 | CE1 | PHE | B | 29 | 121.207 | 98.101 | 55.378 | 1.00 | 13.62 | C |
| ATOM | 1747 | CZ | PHE | B | 29 | 121.736 | 97.997 | 56.664 | 1.00 | 16.22 | C |
| ATOM | 1748 | CE2 | PHE | B | 29 | 123.055 | 97.618 | 56.844 | 1.00 | 15.81 | C |
| ATOM | 1749 | CD2 | PHE | B | 29 | 123.840 | 97.348 | 55.767 | 1.00 | 16.96 | C |
| ATOM | 1750 | C | PHE | B | 29 | 123.559 | 99.017 | 51.733 | 1.00 | 15.35 | C |
| ATOM | 1751 | O | PHE | B | 29 | 123.085 | 98.365 | 50.797 | 1.00 | 14.12 | O |
| ATOM | 1752 | N | MET | B | 30 | 123.166 | 100.241 | 52.057 | 1.00 | 14.79 | N |
| ATOM | 1753 | CA | MET | B | 30 | 122.042 | 100.927 | 51.447 | 1.00 | 14.75 | C |
| ATOM | 1754 | CB | MET | B | 30 | 121.476 | 102.004 | 52.402 | 1.00 | 13.49 | C |
| ATOM | 1755 | CG | MET | B | 30 | 120.355 | 102.821 | 51.842 | 1.00 | 14.77 | C |
| ATOM | 1756 | SD | MET | B | 30 | 118.880 | 101.755 | 51.685 | 1.00 | 11.68 | S |
| ATOM | 1757 | CE | MET | B | 30 | 117.943 | 102.744 | 50.467 | 1.00 | 15.28 | C |
| ATOM | 1758 | C | MET | B | 30 | 122.451 | 101.538 | 50.116 | 1.00 | 15.07 | C |
| ATOM | 1759 | O | MET | B | 30 | 123.445 | 102.250 | 50.015 | 1.00 | 15.43 | O |
| ATOM | 1760 | N | VAL | B | 31 | 121.636 | 101.259 | 49.099 | 1.00 | 15.16 | N |
| ATOM | 1761 | CA | VAL | B | 31 | 121.910 | 101.585 | 47.704 | 1.00 | 14.83 | C |
| ATOM | 1762 | CB | VAL | B | 31 | 122.040 | 100.266 | 46.887 | 1.00 | 14.80 | C |
| ATOM | 1763 | CG1 | VAL | B | 31 | 122.336 | 100.545 | 45.432 | 1.00 | 13.96 | C |
| ATOM | 1764 | CG2 | VAL | B | 31 | 123.096 | 99.364 | 47.464 | 1.00 | 17.40 | C |
| ATOM | 1765 | C | VAL | B | 31 | 120.749 | 102.404 | 47.112 | 1.00 | 14.83 | C |
| ATOM | 1766 | O | VAL | B | 31 | 119.567 | 102.143 | 47.425 | 1.00 | 14.79 | O |
| ATOM | 1767 | N | SER | B | 32 | 121.090 | 103.347 | 46.240 | 1.00 | 14.66 | N |
| ATOM | 1768 | CA | SER | B | 32 | 120.133 | 104.114 | 45.445 | 1.00 | 14.27 | C |
| ATOM | 1769 | CB | SER | B | 32 | 120.458 | 105.598 | 45.489 | 1.00 | 15.14 | C |
| ATOM | 1770 | OG | SER | B | 32 | 119.618 | 106.366 | 44.614 | 1.00 | 14.17 | O |
| ATOM | 1771 | C | SER | B | 32 | 120.209 | 103.640 | 44.003 | 1.00 | 14.08 | C |
| ATOM | 1772 | O | SER | B | 32 | 121.306 | 103.618 | 43.414 | 1.00 | 13.19 | O |
| ATOM | 1773 | N | LEU | B | 33 | 119.057 | 103.214 | 43.462 | 1.00 | 13.54 | N |
| ATOM | 1774 | CA | LEU | B | 33 | 118.882 | 102.912 | 42.052 | 1.00 | 14.12 | C |
| ATOM | 1775 | CB | LEU | B | 33 | 117.861 | 101.777 | 41.844 | 1.00 | 14.60 | C |
| ATOM | 1776 | CG | LEU | B | 33 | 118.329 | 100.332 | 41.670 | 1.00 | 18.35 | C |
| ATOM | 1777 | CD1 | LEU | B | 33 | 119.714 | 99.954 | 42.210 | 1.00 | 16.05 | C |
| ATOM | 1778 | CD2 | LEU | B | 33 | 117.240 | 99.349 | 42.108 | 1.00 | 16.27 | C |
| ATOM | 1779 | C | LEU | B | 33 | 118.411 | 104.176 | 41.347 | 1.00 | 14.85 | C |
| ATOM | 1780 | O | LEU | B | 33 | 117.440 | 104.809 | 41.775 | 1.00 | 14.53 | O |
| ATOM | 1781 | N | GLN | B | 34 | 119.110 | 104.534 | 40.263 | 1.00 | 16.38 | N |
| ATOM | 1782 | CA | GLN | B | 34 | 118.940 | 105.835 | 39.613 | 1.00 | 17.48 | C |
| ATOM | 1783 | CB | GLN | B | 34 | 120.100 | 106.807 | 39.988 | 1.00 | 18.34 | C |
| ATOM | 1784 | CG | GLN | B | 34 | 120.313 | 106.932 | 41.520 | 1.00 | 18.08 | C |
| ATOM | 1785 | CD | GLN | B | 34 | 121.015 | 108.189 | 41.986 | 1.00 | 20.81 | C |
| ATOM | 1786 | OE1 | GLN | B | 34 | 120.989 | 108.501 | 43.186 | 1.00 | 22.00 | O |
| ATOM | 1787 | NE2 | GLN | B | 34 | 121.690 | 108.881 | 41.074 | 1.00 | 16.82 | N |
| ATOM | 1788 | C | GLN | B | 34 | 118.823 | 105.673 | 38.097 | 1.00 | 18.69 | C |
| ATOM | 1789 | O | GLN | B | 34 | 119.416 | 104.779 | 37.502 | 1.00 | 17.09 | O |
| ATOM | 1790 | N | LEU | B | 35 | 118.011 | 106.537 | 37.505 | 1.00 | 21.80 | N |
| ATOM | 1791 | CA | LEU | B | 35 | 117.838 | 106.671 | 36.065 | 1.00 | 24.64 | C |
| ATOM | 1792 | CB | LEU | B | 35 | 116.461 | 106.155 | 35.667 | 1.00 | 25.41 | C |
| ATOM | 1793 | CG | LEU | B | 35 | 116.389 | 104.696 | 35.254 | 1.00 | 26.94 | C |
| ATOM | 1794 | CD1 | LEU | B | 35 | 114.910 | 104.296 | 35.123 | 1.00 | 30.66 | C |
| ATOM | 1795 | CD2 | LEU | B | 35 | 117.125 | 104.490 | 33.955 | 1.00 | 29.13 | C |
| ATOM | 1796 | C | LEU | B | 35 | 117.932 | 108.160 | 35.683 | 1.00 | 26.93 | C |
| ATOM | 1797 | O | LEU | B | 35 | 117.153 | 108.976 | 36.178 | 1.00 | 27.47 | O |
| ATOM | 1798 | N | ARG | B | 36 | 118.827 | 108.461 | 34.746 | 1.00 | 29.79 | N |
| ATOM | 1799 | CA | ARG | B | 36 | 119.420 | 109.808 | 34.510 | 1.00 | 31.41 | C |
| ATOM | 1800 | CB | ARG | B | 36 | 118.886 | 110.484 | 33.231 | 1.00 | 32.91 | C |
| ATOM | 1801 | CG | ARG | B | 36 | 120.014 | 111.296 | 32.478 | 1.00 | 37.26 | C |
| ATOM | 1802 | CD | ARG | B | 36 | 119.531 | 112.408 | 31.511 | 1.00 | 42.36 | C |
| ATOM | 1803 | NE | ARG | B | 36 | 119.886 | 113.746 | 32.024 | 1.00 | 47.18 | N |
| ATOM | 1804 | CZ | ARG | B | 36 | 120.602 | 114.674 | 31.374 | 1.00 | 50.50 | C |
| ATOM | 1805 | NH1 | ARG | B | 36 | 121.057 | 114.454 | 30.132 | 1.00 | 51.16 | N |
| ATOM | 1806 | NH2 | ARG | B | 36 | 120.861 | 115.845 | 31.971 | 1.00 | 51.17 | N |
| ATOM | 1807 | C | ARG | B | 36 | 119.363 | 110.756 | 35.695 | 1.00 | 30.67 | C |
| ATOM | 1808 | O | ARG | B | 36 | 118.568 | 111.694 | 35.733 | 1.00 | 31.07 | O |
| ATOM | 1809 | N | GLY | B | 38 | 120.217 | 110.456 | 36.672 | 1.00 | 30.19 | N |
| ATOM | 1810 | CA | GLY | B | 38 | 120.426 | 111.279 | 37.854 | 1.00 | 29.41 | C |
| ATOM | 1811 | C | GLY | B | 38 | 119.377 | 111.209 | 38.954 | 1.00 | 28.01 | C |
| ATOM | 1812 | O | GLY | B | 38 | 119.601 | 111.771 | 40.034 | 1.00 | 28.90 | O |
| ATOM | 1813 | N | GLY | B | 39 | 118.247 | 110.538 | 38.700 | 1.00 | 25.24 | N |
| ATOM | 1814 | CA | GLY | B | 39 | 117.108 | 110.558 | 39.607 | 1.00 | 22.49 | C |
| ATOM | 1815 | C | GLY | B | 39 | 116.825 | 109.207 | 40.267 | 1.00 | 19.97 | C |
| ATOM | 1816 | O | GLY | B | 39 | 116.658 | 108.203 | 39.572 | 1.00 | 18.01 | O |
| ATOM | 1817 | N | HIS | B | 40 | 116.839 | 109.215 | 41.602 | 1.00 | 18.22 | N |
| ATOM | 1818 | CA | HIS | B | 40 | 116.468 | 108.089 | 42.478 | 1.00 | 15.80 | C |
| ATOM | 1819 | CB | HIS | B | 40 | 116.558 | 108.521 | 43.946 | 1.00 | 15.79 | C |
| ATOM | 1820 | CG | HIS | B | 40 | 116.149 | 107.440 | 44.908 | 1.00 | 14.56 | C |
| ATOM | 1821 | ND1 | HIS | B | 40 | 114.826 | 107.141 | 45.178 | 1.00 | 18.28 | N |
| ATOM | 1822 | CE1 | HIS | B | 40 | 114.774 | 106.126 | 46.025 | 1.00 | 14.76 | C |
| ATOM | 1823 | NE2 | HIS | B | 40 | 116.010 | 105.741 | 46.292 | 1.00 | 15.85 | N |
| ATOM | 1824 | CD2 | HIS | B | 40 | 116.884 | 106.548 | 45.609 | 1.00 | 13.05 | C |
| ATOM | 1825 | C | HIS | B | 40 | 115.046 | 107.576 | 42.180 | 1.00 | 14.80 | C |
| ATOM | 1826 | O | HIS | B | 40 | 114.127 | 108.360 | 42.107 | 1.00 | 14.37 | O |
| ATOM | 1827 | N | PHE | B | 41 | 114.880 | 106.260 | 42.007 | 1.00 | 13.74 | N |
| ATOM | 1828 | CA | PHE | B | 41 | 113.526 | 105.669 | 41.827 | 1.00 | 13.67 | C |
| ATOM | 1829 | CB | PHE | B | 41 | 113.276 | 105.289 | 40.346 | 1.00 | 13.07 | C |
| ATOM | 1830 | CG | PHE | B | 41 | 114.161 | 104.222 | 39.829 | 1.00 | 13.73 | C |
| ATOM | 1831 | CD1 | PHE | B | 41 | 113.760 | 102.895 | 39.885 | 1.00 | 14.36 | C |
| ATOM | 1832 | CE1 | PHE | B | 41 | 114.565 | 101.907 | 39.408 | 1.00 | 16.20 | C |
| ATOM | 1833 | CZ | PHE | B | 41 | 115.797 | 102.194 | 38.840 | 1.00 | 13.36 | C |
| ATOM | 1834 | CE2 | PHE | B | 41 | 116.186 | 103.505 | 38.750 | 1.00 | 16.12 | C |
| ATOM | 1835 | CD2 | PHE | B | 41 | 115.368 | 104.515 | 39.239 | 1.00 | 13.10 | C |
| ATOM | 1836 | C | PHE | B | 41 | 113.268 | 104.502 | 42.791 | 1.00 | 13.67 | C |
| ATOM | 1837 | O | PHE | B | 41 | 112.122 | 104.202 | 43.110 | 1.00 | 13.12 | O |
| ATOM | 1838 | N | CYS | B | 42 | 114.321 | 103.854 | 43.275 | 1.00 | 13.33 | N |
| ATOM | 1839 | CA | CYS | B | 42 | 114.150 | 102.750 | 44.234 | 1.00 | 12.99 | C |
| ATOM | 1840 | CB | CYS | B | 42 | 113.870 | 101.458 | 43.489 | 1.00 | 13.38 | C |
| ATOM | 1841 | SG | CYS | B | 42 | 112.130 | 101.191 | 43.092 | 1.00 | 11.77 | S |
| ATOM | 1842 | C | CYS | B | 42 | 115.418 | 102.567 | 45.063 | 1.00 | 13.54 | C |
| ATOM | 1843 | O | CYS | B | 42 | 116.487 | 103.045 | 44.677 | 1.00 | 14.09 | O |
| ATOM | 1844 | N | GLY | B | 43 | 115.268 | 101.928 | 46.222 | 1.00 | 12.74 | N |
| ATOM | 1845 | CA | GLY | B | 43 | 116.400 | 101.507 | 47.026 | 1.00 | 12.28 | C |
| ATOM | 1846 | C | GLY | B | 43 | 116.860 | 100.138 | 46.568 | 1.00 | 11.55 | C |
| ATOM | 1847 | O | GLY | B | 43 | 116.232 | 99.447 | 45.765 | 1.00 | 12.41 | O |
| ATOM | 1848 | N | ALA | B | 44 | 117.973 | 99.744 | 47.121 | 1.00 | 12.23 | N |
| ATOM | 1849 | CA | ALA | B | 44 | 118.466 | 98.387 | 47.048 | 1.00 | 11.95 | C |
| ATOM | 1850 | CB | ALA | B | 44 | 119.147 | 98.121 | 45.715 | 1.00 | 13.44 | C |
| ATOM | 1851 | C | ALA | B | 44 | 119.416 | 98.153 | 48.230 | 1.00 | 12.34 | C |
| ATOM | 1852 | O | ALA | B | 44 | 119.707 | 99.081 | 48.997 | 1.00 | 12.72 | O |
| ATOM | 1853 | N | THR | B | 45 | 119.903 | 96.925 | 48.322 | 1.00 | 12.23 | N |
| ATOM | 1854 | CA | THR | B | 45 | 120.848 | 96.454 | 49.330 | 1.00 | 12.89 | C |
| ATOM | 1855 | CB | THR | B | 45 | 120.117 | 95.449 | 50.246 | 1.00 | 12.86 | C |
| ATOM | 1856 | OG1 | THR | B | 45 | 118.980 | 96.097 | 50.847 | 1.00 | 11.92 | O |
| ATOM | 1857 | CG2 | THR | B | 45 | 121.020 | 95.000 | 51.397 | 1.00 | 12.68 | C |
| ATOM | 1858 | C | THR | B | 45 | 122.046 | 95.714 | 48.713 | 1.00 | 12.73 | C |
| ATOM | 1859 | O | THR | B | 45 | 121.888 | 94.824 | 47.903 | 1.00 | 13.04 | O |
| ATOM | 1860 | N | LEU | B | 46 | 123.260 | 96.068 | 49.133 | 1.00 | 13.34 | N |
| ATOM | 1861 | CA | LEU | B | 46 | 124.466 | 95.334 | 48.737 | 1.00 | 13.63 | C |
| ATOM | 1862 | CB | LEU | B | 46 | 125.692 | 96.232 | 49.009 | 1.00 | 14.23 | C |
| ATOM | 1863 | CG | LEU | B | 46 | 126.987 | 95.820 | 48.360 | 1.00 | 15.02 | C |
| ATOM | 1864 | CD1 | LEU | B | 46 | 126.871 | 95.929 | 46.863 | 1.00 | 16.32 | C |
| ATOM | 1865 | CD2 | LEU | B | 46 | 128.136 | 96.755 | 48.869 | 1.00 | 13.91 | C |
| ATOM | 1866 | C | LEU | B | 46 | 124.559 | 94.023 | 49.530 | 1.00 | 14.01 | C |
| ATOM | 1867 | O | LEU | B | 46 | 124.644 | 94.033 | 50.791 | 1.00 | 14.91 | O |
| ATOM | 1868 | N | ILE | B | 47 | 124.523 | 92.886 | 48.828 | 1.00 | 14.19 | N |
| ATOM | 1869 | CA | ILE | B | 47 | 124.578 | 91.571 | 49.472 | 1.00 | 14.09 | C |
| ATOM | 1870 | CB | ILE | B | 47 | 123.273 | 90.763 | 49.152 | 1.00 | 14.08 | C |
| ATOM | 1871 | CG1 | ILE | B | 47 | 123.063 | 90.602 | 47.644 | 1.00 | 14.29 | C |
| ATOM | 1872 | CD1 | ILE | B | 47 | 121.932 | 89.633 | 47.289 | 1.00 | 14.18 | C |
| ATOM | 1873 | CG2 | ILE | B | 47 | 122.077 | 91.488 | 49.778 | 1.00 | 14.43 | C |
| ATOM | 1874 | C | ILE | B | 47 | 125.822 | 90.770 | 49.103 | 1.00 | 14.96 | C |
| ATOM | 1875 | O | ILE | B | 47 | 125.987 | 89.644 | 49.566 | 1.00 | 15.41 | O |
| ATOM | 1876 | N | ALA | B | 48 | 126.628 | 91.333 | 48.203 | 1.00 | 15.70 | N |
| ATOM | 1877 | CA | ALA | B | 48 | 127.987 | 90.874 | 47.927 | 1.00 | 15.64 | C |
| ATOM | 1878 | CB | ALA | B | 48 | 127.986 | 89.640 | 47.071 | 1.00 | 15.53 | C |
| ATOM | 1879 | C | ALA | B | 48 | 128.688 | 92.018 | 47.238 | 1.00 | 16.47 | C |
| ATOM | 1880 | O | ALA | B | 48 | 128.044 | 92.941 | 46.792 | 1.00 | 15.98 | O |
| ATOM | 1881 | N | PRO | B | 49 | 130.014 | 91.993 | 47.120 | 1.00 | 16.92 | N |
| ATOM | 1882 | CA | PRO | B | 49 | 130.705 | 93.088 | 46.424 | 1.00 | 16.70 | C |
| ATOM | 1883 | CB | PRO | B | 49 | 132.140 | 92.568 | 46.308 | 1.00 | 16.25 | C |
| ATOM | 1884 | CG | PRO | B | 49 | 132.251 | 91.616 | 47.492 | 1.00 | 18.06 | C |
| ATOM | 1885 | CD | PRO | B | 49 | 130.946 | 90.971 | 47.622 | 1.00 | 17.73 | C |
| ATOM | 1886 | C | PRO | B | 49 | 130.127 | 93.388 | 45.038 | 1.00 | 15.87 | C |
| ATOM | 1887 | O | PRO | B | 49 | 130.095 | 94.555 | 44.659 | 1.00 | 15.76 | O |
| ATOM | 1888 | N | ASN | B | 50 | 129.682 | 92.345 | 44.334 | 1.00 | 16.42 | N |
| ATOM | 1889 | CA | ASN | B | 50 | 129.163 | 92.463 | 42.955 | 1.00 | 16.13 | C |
| ATOM | 1890 | CB | ASN | B | 50 | 129.999 | 91.626 | 41.990 | 1.00 | 15.49 | C |
| ATOM | 1891 | CG | ASN | B | 50 | 129.874 | 90.176 | 42.246 | 1.00 | 16.71 | C |
| ATOM | 1892 | OD1 | ASN | B | 50 | 129.722 | 89.751 | 43.396 | 1.00 | 20.77 | O |
| ATOM | 1893 | ND2 | ASN | B | 50 | 129.950 | 89.378 | 41.191 | 1.00 | 19.26 | N |
| ATOM | 1894 | C | ASN | B | 50 | 127.697 | 92.072 | 42.775 | 1.00 | 15.32 | C |
| ATOM | 1895 | O | ASN | B | 50 | 127.294 | 91.712 | 41.675 | 1.00 | 15.03 | O |
| ATOM | 1896 | N | PHE | B | 51 | 126.891 | 92.205 | 43.834 | 1.00 | 15.54 | N |
| ATOM | 1897 | CA | PHE | B | 51 | 125.452 | 91.922 | 43.735 | 1.00 | 13.91 | C |
| ATOM | 1898 | CB | PHE | 5 | 51 | 125.135 | 90.495 | 44.144 | 1.00 | 13.59 | C |
| ATOM | 1899 | CG | PHE | B | 51 | 125.524 | 89.445 | 43.140 | 1.00 | 15.33 | C |
| ATOM | 1900 | CD1 | PHE | B | 51 | 124.631 | 89.041 | 42.142 | 1.00 | 17.09 | C |
| ATOM | 1901 | CE1 | PHE | B | 51 | 124.955 | 88.030 | 41.248 | 1.00 | 18.18 | C |
| ATOM | 1902 | CZ | PHE | B | 51 | 126.200 | 87.408 | 41.322 | 1.00 | 19.09 | C |
| ATOM | 1903 | CE2 | PHE | B | 51 | 127.083 | 87.786 | 42.296 | 1.00 | 18.35 | C |
| ATOM | 1904 | CD2 | PHE | B | 51 | 126.748 | 88.800 | 43.215 | 1.00 | 14.76 | C |
| ATOM | 1905 | C | PHE | B | 51 | 124.662 | 92.841 | 44.636 | 1.00 | 13.80 | C |
| ATOM | 1906 | O | PHE | B | 51 | 125.072 | 93.121 | 45.810 | 1.00 | 11.89 | O |
| ATOM | 1907 | N | VAL | B | 52 | 123.517 | 93.326 | 44.106 | 1.00 | 12.49 | N |
| ATOM | 1908 | CA | VAL | B | 52 | 122.517 | 93.941 | 44.946 | 1.00 | 11.63 | C |
| ATOM | 1909 | CB | VAL | B | 52 | 122.295 | 95.449 | 44.670 | 1.00 | 11.23 | C |
| ATOM | 1910 | CG1 | VAL | B | 52 | 121.675 | 95.709 | 43.335 | 1.00 | 10.85 | C |
| ATOM | 1911 | CG2 | VAL | B | 52 | 123.619 | 96.267 | 44.796 | 1.00 | 12.66 | C |
| ATOM | 1912 | C | VAL | B | 52 | 121.205 | 93.178 | 44.847 | 1.00 | 10.42 | C |
| ATOM | 1913 | O | VAL | B | 52 | 120.975 | 92.418 | 43.905 | 1.00 | 10.37 | O |
| ATOM | 1914 | N | MET | B | 53 | 120.353 | 93.369 | 45.830 | 1.00 | 11.29 | N |
| ATOM | 1915 | CA | MET | B | 53 | 118.965 | 92.917 | 45.739 | 1.00 | 10.46 | C |
| ATOM | 1916 | CB | MET | B | 53 | 118.615 | 91.763 | 46.712 | 1.00 | 11.61 | C |
| ATOM | 1917 | CG | MET | B | 53 | 118.427 | 92.127 | 48.111 | 1.00 | 13.11 | C |
| ATOM | 1918 | SD | MET | B | 53 | 118.170 | 90.666 | 49.192 | 1.00 | 14.64 | S |
| ATOM | 1919 | CE | MET | B | 53 | 118.245 | 91.580 | 50.657 | 1.00 | 14.12 | C |
| ATOM | 1920 | C | MET | B | 53 | 118.039 | 94.091 | 45.938 | 1.00 | 10.08 | C |
| ATOM | 1921 | O | MET | B | 53 | 118.303 | 95.009 | 46.721 | 1.00 | 11.93 | O |
| ATOM | 1922 | N | SER | B | 54 | 116.926 | 94.041 | 45.208 | 1.00 | 10.47 | N |
| ATOM | 1923 | CA | SER | B | 54 | 115.900 | 95.059 | 45.248 | 1.00 | 9.01 | C |
| ATOM | 1924 | CB | SER | B | 54 | 116.221 | 96.096 | 44.178 | 1.00 | 9.81 | C |
| ATOM | 1925 | OG | SER | B | 54 | 115.420 | 97.230 | 44.299 | 1.00 | 9.00 | O |
| ATOM | 1926 | C | SER | B | 54 | 114.529 | 94.444 | 45.011 | 1.00 | 9.05 | C |
| ATOM | 1927 | O | SER | B | 54 | 114.380 | 93.218 | 45.042 | 1.00 | 6.87 | O |
| ATOM | 1928 | N | ALA | B | 55 | 113.518 | 95.305 | 44.772 | 1.00 | 9.77 | N |
| ATOM | 1929 | CA | ALA | B | 55 | 112.166 | 94.806 | 44.489 | 1.00 | 9.48 | C |
| ATOM | 1930 | CB | ALA | B | 55 | 111.155 | 95.780 | 44.963 | 1.00 | 10.16 | C |
| ATOM | 1931 | C | ALA | B | 55 | 112.047 | 94.627 | 42.986 | 1.00 | 9.40 | C |
| ATOM | 1932 | O | ALA | B | 55 | 112.430 | 95.532 | 42.233 | 1.00 | 8.93 | O |
| ATOM | 1933 | N | ALA | B | 56 | 111.528 | 93.485 | 42.540 | 1.00 | 9.20 | N |
| ATOM | 1934 | CA | ALA | B | 56 | 111.382 | 93.216 | 41.101 | 1.00 | 9.96 | C |
| ATOM | 1935 | CB | ALA | B | 56 | 110.726 | 91.853 | 40.855 | 1.00 | 10.73 | C |
| ATOM | 1936 | C | ALA | B | 56 | 110.638 | 94.308 | 40.358 | 1.00 | 10.18 | C |
| ATOM | 1937 | O | ALA | B | 56 | 111.003 | 94.677 | 39.209 | 1.00 | 8.94 | O |
| ATOM | 1938 | N | HIS | B | 57 | 109.661 | 94.921 | 41.021 | 1.00 | 10.83 | N |
| ATOM | 1939 | CA | HIS | B | 57 | 108.818 | 95.908 | 40.334 | 1.00 | 10.77 | C |
| ATOM | 1940 | CB | HIS | B | 57 | 107.532 | 96.200 | 41.103 | 1.00 | 10.86 | C |
| ATOM | 1941 | CG | HIS | B | 57 | 107.691 | 97.102 | 42.283 | 1.00 | 9.98 | C |
| ATOM | 1942 | ND1 | HIS | B | 57 | 107.766 | 98.468 | 42.172 | 1.00 | 12.54 | N |
| ATOM | 1943 | CE1 | HIS | B | 57 | 107.895 | 99.000 | 43.381 | 1.00 | 13.05 | C |
| ATOM | 1944 | NE2 | HIS | B | 57 | 107.843 | 98.027 | 44.275 | 1.00 | 15.44 | N |
| ATOM | 1945 | CD2 | HIS | B | 57 | 107.722 | 96.832 | 43.610 | 1.00 | 9.24 | C |
| ATOM | 1946 | C | HIS | B | 57 | 109.580 | 97.182 | 40.017 | 1.00 | 12.25 | C |
| ATOM | 1947 | O | HIS | B | 57 | 109.195 | 97.956 | 39.125 | 1.00 | 12.23 | O |
| ATOM | 1948 | N | CYS | B | 58 | 110.670 | 97.398 | 40.757 | 1.00 | 12.17 | N |
| ATOM | 1949 | CA | CYS | B | 58 | 111.562 | 98.520 | 40.489 | 1.00 | 12.57 | C |
| ATOM | 1950 | CB | CYS | B | 58 | 112.646 | 98.594 | 41.559 | 1.00 | 12.19 | C |
| ATOM | 1951 | SG | CYS | B | 58 | 112.018 | 99.150 | 43.172 | 1.00 | 12.41 | 5 |
| ATOM | 1952 | C | CYS | B | 58 | 112.192 | 98.455 | 39.101 | 1.00 | 12.77 | C |
| ATOM | 1953 | O | CYS | B | 58 | 112.455 | 99.502 | 38.487 | 1.00 | 14.98 | O |
| ATOM | 1954 | N | VAL | B | 59 | 112.469 | 97.257 | 38.599 | 1.00 | 12.63 | N |
| ATOM | 1955 | CA | VAL | B | 59 | 113.103 | 97.151 | 37.299 | 1.00 | 13.72 | C |
| ATOM | 1956 | CB | VAL | B | 59 | 114.366 | 96.283 | 37.298 | 1.00 | 14.02 | C |
| ATOM | 1957 | CG1 | VAL | B | 59 | 115.416 | 96.922 | 38.178 | 1.00 | 15.70 | C |
| ATOM | 1958 | CG2 | VAL | B | 59 | 114.061 | 94.854 | 37.701 | 1.00 | 15.65 | C |
| ATOM | 1959 | C | VAL | B | 59 | 112.166 | 96.732 | 36.187 | 1.00 | 14.49 | C |
| ATOM | 1960 | O | VAL | B | 59 | 112.612 | 96.450 | 35.067 | 1.00 | 14.74 | O |
| ATOM | 1961 | N | ALA | B | 60 | 110.872 | 96.692 | 36.474 | 1.00 | 14.93 | N |
| ATOM | 1962 | CA | ALA | B | 60 | 109.896 | 96.398 | 35.435 | 1.00 | 16.47 | C |
| ATOM | 1963 | CB | ALA | B | 60 | 108.609 | 95.926 | 36.060 | 1.00 | 17.08 | C |
| ATOM | 1964 | C | ALA | B | 60 | 109.645 | 97.653 | 34.600 | 1.00 | 16.69 | C |
| ATOM | 1965 | O | ALA | B | 60 | 109.583 | 98.761 | 35.126 | 1.00 | 17.56 | O |
| ATOM | 1966 | N | ASN | B | 61 | 109.502 | 97.516 | 33.295 | 1.00 | 17.18 | N |
| ATOM | 1967 | CA | ASN | B | 61 | 109.118 | 98.687 | 32.483 | 1.00 | 17.26 | C |
| ATOM | 1958 | CB | ASN | B | 61 | 107.687 | 99.161 | 32.786 | 1.00 | 18.41 | C |
| ATOM | 1969 | CG | ASN | B | 61 | 107.082 | 99.874 | 31.587 | 1.00 | 15.88 | C |
| ATOM | 1970 | OD1 | ASN | B | 61 | 107.629 | 99.771 | 30.517 | 1.00 | 18.03 | O |
| ATOM | 1971 | ND2 | ASN | B | 61 | 105.972 | 100.585 | 31.762 | 1.00 | 17.99 | N |
| ATOM | 1972 | C | ASN | B | 61 | 110.076 | 99.910 | 32.587 | 1.00 | 17.76 | C |
| ATOM | 1973 | O | ASN | B | 61 | 109.654 | 101.045 | 32.590 | 1.00 | 17.51 | O |
| ATOM | 1974 | N | VAL | B | 62 | 111.364 | 99.624 | 32.705 | 1.00 | 18.71 | N |
| ATOM | 1975 | CA | VAL | B | 62 | 112.407 | 100.635 | 32.670 | 1.00 | 18.69 | C |
| ATOM | 1976 | CB | VAL | B | 62 | 112.948 | 100.931 | 34.052 | 1.00 | 20.07 | C |
| ATOM | 1977 | CG1 | VAL | B | 62 | 111.828 | 101.371 | 34.970 | 1.00 | 22.93 | C |
| ATOM | 1978 | CG2 | VAL | B | 62 | 113.667 | 99.695 | 34.649 | 1.00 | 19.01 | C |
| ATOM | 1979 | C | VAL | B | 62 | 113.517 | 100.127 | 31.774 | 1.00 | 18.98 | C |
| ATOM | 1980 | O | VAL | B | 62 | 113.559 | 98.940 | 31.410 | 1.00 | 18.25 | O |
| ATOM | 1981 | N | ASN | B | 62A | 114.400 | 101.024 | 31.363 | 1.00 | 18.75 | N |
| ATOM | 1982 | CA | ASN | B | 62A | 115.555 | 100.596 | 30.588 | 1.00 | 19.09 | C |
| ATOM | 1983 | CB | ASN | B | 62A | 116.029 | 101.683 | 29.628 | 1.00 | 19.48 | C |
| ATOM | 1984 | CG | ASN | B | 62A | 117.273 | 101.266 | 28.847 | 1.00 | 19.35 | C |
| ATOM | 1985 | OD1 | ASN | B | 62A | 117.605 | 100.096 | 28.791 | 1.00 | 21.72 | O |
| ATOM | 1986 | ND2 | ASN | B | 62A | 117.963 | 102.243 | 28.239 | 1.00 | 18.71 | N |
| ATOM | 1987 | C | ASN | B | 62A | 116.647 | 100.212 | 31.577 | 1.00 | 19.63 | C |
| ATOM | 1988 | O | ASN | B | 62A | 117.362 | 101.088 | 32.090 | 1.00 | 18.11 | O |
| ATOM | 1989 | N | VAL | B | 62B | 116.763 | 98.900 | 31.840 | 1.00 | 20.19 | N |
| ATOM | 1990 | CA | VAL | B | 62B | 117.719 | 98.424 | 32.840 | 1.00 | 21.11 | C |
| ATOM | 1991 | CB | VAL | B | 62B | 117.599 | 96.918 | 33.121 | 1.00 | 21.70 | C |
| ATOM | 1992 | CG1 | VAL | B | 62B | 118.624 | 96.500 | 34.132 | 1.00 | 23.87 | C |
| ATOM | 1993 | CG2 | VAL | B | 62B | 116.205 | 96.557 | 33.660 | 1.00 | 23.53 | C |
| ATOM | 1994 | C | VAL | B | 62B | 119.165 | 98.733 | 32.447 | 1.00 | 20.51 | C |
| ATOM | 1995 | O | VAL | B | 62B | 120.009 | 98.928 | 33.317 | 1.00 | 19.89 | O |
| ATOM | 1996 | N | ARG | B | 63 | 119.437 | 98.852 | 31.160 | 1.00 | 20.74 | N |
| ATOM | 1997 | CA | ARG | B | 63 | 120.805 | 99.179 | 30.705 | 1.00 | 20.85 | C |
| ATOM | 1998 | CB | ARG | B | 63 | 120.921 | 99.097 | 29.186 | 1.00 | 21.82 | C |
| ATOM | 1999 | CG | ARG | B | 63 | 120.632 | 97.748 | 28.580 | 1.00 | 26.58 | C |
| ATOM | 2000 | CD | ARG | B | 63 | 120.804 | 97.692 | 27.046 | 1.00 | 31.80 | C |
| ATOM | 2001 | NE | ARG | B | 63 | 119.839 | 98.578 | 26.379 | 1.00 | 35.15 | N |
| ATOM | 2002 | CZ | ARG | B | 63 | 120.138 | 99.719 | 25.741 | 1.00 | 41.26 | C |
| ATOM | 2003 | NH1 | ARG | B | 63 | 121.401 | 100.160 | 25.648 | 1.00 | 44.51 | N |
| ATOM | 2004 | NH2 | ARG | B | 63 | 119.162 | 100.433 | 25.181 | 1.00 | 42.92 | N |
| ATOM | 2005 | C | ARG | B | 63 | 121.205 | 100.565 | 31.147 | 1.00 | 19.74 | C |
| ATOM | 2006 | O | ARG | B | 63 | 122.402 | 100.874 | 31.259 | 1.00 | 19.47 | O |
| ATOM | 2007 | N | ALA | B | 64 | 120.218 | 101.426 | 31.370 | 1.00 | 19.22 | N |
| ATOM | 2008 | CA | ALA | B | 64 | 120.463 | 102.780 | 31.829 | 1.00 | 19.45 | C |
| ATOM | 2009 | CB | ALA | B | 64 | 119.496 | 103.741 | 31.158 | 1.00 | 20.13 | C |
| ATOM | 2010 | C | ALA | B | 64 | 120.418 | 102.966 | 33.362 | 1.00 | 19.38 | C |
| ATOM | 2011 | O | ALA | B | 64 | 120.649 | 104.065 | 33.855 | 1.00 | 18.24 | O |
| ATOM | 2012 | N | VAL | B | 65 | 120.102 | 101.911 | 34.102 | 1.00 | 18.84 | N |
| ATOM | 2013 | CA | VAL | B | 65 | 120.047 | 102.003 | 35.558 | 1.00 | 18.66 | C |
| ATOM | 2014 | CB | VAL | B | 65 | 119.354 | 100.817 | 36.212 | 1.00 | 18.00 | C |
| ATOM | 2015 | CG1 | VAL | B | 65 | 119.495 | 100.928 | 37.753 | 1.00 | 19.47 | C |
| ATOM | 2016 | CG2 | VAL | B | 65 | 117.901 | 100.788 | 35.813 | 1.00 | 20.73 | C |
| ATOM | 2017 | C | VAL | B | 65 | 121.451 | 102.121 | 36.159 | 1.00 | 18.40 | C |
| ATOM | 2018 | O | VAL | B | 65 | 122.362 | 101.343 | 35.837 | 1.00 | 18.18 | O |
| ATOM | 2019 | N | ARG | B | 65A | 121.593 | 103.107 | 37.026 | 1.00 | 18.97 | N |
| ATOM | 2020 | CA | ARG | B | 65A | 122.811 | 103.296 | 37.787 | 1.00 | 19.21 | C |
| ATOM | 2021 | CB | ARG | B | 65A | 123.256 | 104.756 | 37.707 | 1.00 | 19.95 | C |
| ATOM | 2022 | CG | ARG | B | 65A | 123.855 | 105.100 | 36.306 | 1.00 | 25.32 | C |
| ATOM | 2023 | CD | ARG | B | 65A | 124.837 | 104.018 | 35.763 | 1.00 | 32.74 | C |
| ATOM | 2024 | NE | ARG | B | 65A | 124.558 | 103.465 | 34.413 | 1.00 | 39.31 | N |
| ATOM | 2025 | CZ | ARG | B | 65A | 125.384 | 103.565 | 33.356 | 1.00 | 42.08 | C |
| ATOM | 2026 | NH1 | ARG | B | 65A | 126.542 | 104.209 | 33.466 | 1.00 | 41.81 | N |
| ATOM | 2027 | NH2 | ARG | B | 65A | 125.044 | 103.033 | 32.173 | 1.00 | 44.09 | N |
| ATOM | 2028 | C | ARG | B | 65A | 122.586 | 102.840 | 39.230 | 1.00 | 18.04 | C |
| ATOM | 2029 | O | ARG | B | 65A | 121.607 | 103.201 | 39.870 | 1.00 | 17.94 | O |
| ATOM | 2030 | N | VAL | B | 66 | 123.516 | 102.046 | 39.716 | 1.00 | 16.15 | N |
| ATOM | 2031 | CA | VAL | B | 66 | 123.441 | 101.467 | 41.039 | 1.00 | 15.45 | C |
| ATOM | 2032 | CB | VAL | B | 66 | 123.847 | 99.995 | 40.979 | 1.00 | 15.69 | C |
| ATOM | 2033 | CG1 | VAL | B | 66 | 123.767 | 99.334 | 42.367 | 1.00 | 15.17 | C |
| ATOM | 2034 | CG2 | VAL | B | 66 | 122.992 | 99.248 | 39.964 | 1.00 | 15.88 | C |
| ATOM | 2035 | C | VAL | B | 66 | 124.433 | 102.250 | 41.873 | 1.00 | 15.56 | C |
| ATOM | 2036 | O | VAL | B | 66 | 125.646 | 102.086 | 41.687 | 1.00 | 16.20 | O |
| ATOM | 2037 | N | VAL | B | 67 | 123.930 | 103.118 | 42.742 | 1.00 | 14.84 | N |
| ATOM | 2038 | CA | VAL | B | 67 | 124.761 | 104.035 | 43.482 | 1.00 | 15.72 | C |
| ATOM | 2039 | CB | VAL | B | 67 | 124.134 | 105.430 | 43.472 | 1.00 | 15.77 | C |
| ATOM | 2040 | CG1 | VAL | B | 67 | 125.040 | 106.426 | 44.192 | 1.00 | 16.26 | C |
| ATOM | 2041 | CG2 | VAL | B | 67 | 123.839 | 105.883 | 42.058 | 1.00 | 16.29 | C |
| ATOM | 2042 | C | VAL | B | 67 | 124.978 | 103.556 | 44.941 | 1.00 | 16.06 | C |
| ATOM | 2043 | O | VAL | B | 67 | 124.045 | 103.565 | 45.747 | 1.00 | 16.28 | O |
| ATOM | 2044 | N | LEU | B | 68 | 126.207 | 103.131 | 45.226 | 1.00 | 16.95 | N |
| ATOM | 2045 | CA | LEU | B | 68 | 126.672 | 102.728 | 46.570 | 1.00 | 17.43 | C |
| ATOM | 2046 | CB | LEU | B | 68 | 127.711 | 101.622 | 46.497 | 1.00 | 17.10 | C |
| ATOM | 2047 | CG | LEU | B | 68 | 127.479 | 100.538 | 45.457 | 1.00 | 19.36 | C |
| ATOM | 2048 | CD1 | LEU | B | 68 | 128.605 | 99.517 | 45.478 | 1.00 | 17.33 | C |
| ATOM | 2049 | CD2 | LEU | B | 68 | 126.155 | 99.896 | 45.718 | 1.00 | 19.81 | C |
| ATOM | 2050 | C | LEU | B | 68 | 127.314 | 103.906 | 47.299 | 1.00 | 17.63 | C |
| ATOM | 2051 | O | LEU | B | 68 | 127.805 | 104.830 | 46.666 | 1.00 | 17.60 | O |
| ATOM | 2052 | N | GLY | B | 69 | 127.289 | 103.889 | 48.629 | 1.00 | 17.78 | N |
| ATOM | 2053 | CA | GLY | B | 69 | 128.007 | 104.893 | 49.404 | 1.00 | 18.14 | C |
| ATOM | 2054 | C | GLY | B | 69 | 127.398 | 106.275 | 49.499 | 1.00 | 18.61 | C |
| ATOM | 2055 | O | GLY | B | 69 | 128.101 | 107.220 | 49.867 | 1.00 | 18.81 | O |
| ATOM | 2056 | N | ALA | B | 70 | 126.109 | 106.410 | 49.197 | 1.00 | 17.79 | N |
| ATOM | 2057 | CA | ALA | B | 70 | 125.469 | 107.709 | 49.204 | 1.00 | 19.28 | C |
| ATOM | 2058 | CB | ALA | B | 70 | 124.511 | 107.840 | 48.048 | 1.00 | 19.21 | C |
| ATOM | 2059 | C | ALA | B | 70 | 124.764 | 108.008 | 50.528 | 1.00 | 19.93 | C |
| ATOM | 2060 | O | ALA | B | 70 | 124.451 | 107.102 | 51.284 | 1.00 | 19.46 | O |
| ATOM | 2061 | N | HIS | B | 71 | 124.576 | 109.297 | 50.791 | 1.00 | 20.20 | N |
| ATOM | 2062 | CA | HIS | B | 71 | 123.805 | 109.762 | 51.913 | 1.00 | 21.17 | C |
| ATOM | 2063 | CB | HIS | B | 71 | 124.704 | 110.227 | 53.030 | 1.00 | 21.92 | C |
| ATOM | 2064 | CG | HIS | B | 71 | 123.949 | 110.623 | 54.254 | 1.00 | 22.20 | C |
| ATOM | 2065 | ND1 | HIS | B | 71 | 123.385 | 109.700 | 55.099 | 1.00 | 20.43 | N |
| ATOM | 2066 | CE1 | HIS | B | 71 | 122.794 | 110.329 | 56.097 | 1.00 | 23.77 | C |
| ATOM | 2067 | NE2 | HIS | B | 71 | 122.918 | 111.631 | 55.908 | 1.00 | 22.11 | N |
| ATOM | 2068 | CD2 | HIS | B | 71 | 123.626 | 111.842 | 54.751 | 1.00 | 24.37 | C |
| ATOM | 2069 | C | HIS | B | 71 | 122.852 | 110.870 | 51.567 | 1.00 | 21.21 | C |
| ATOM | 2070 | O | HIS | B | 71 | 121.669 | 110.737 | 51.808 | 1.00 | 21.15 | O |
| ATOM | 2071 | N | ASN | B | 72 | 123.376 | 111.979 | 51.031 | 1.00 | 21.65 | N |
| ATOM | 2072 | CA | ASN | B | 72 | 122.582 | 113.113 | 50.604 | 1.00 | 21.31 | C |
| ATOM | 2073 | CB | ASN | B | 72 | 123.129 | 114.415 | 51.236 | 1.00 | 21.49 | C |
| ATOM | 2074 | CG | ASN | B | 72 | 122.234 | 115.644 | 50.990 | 1.00 | 21.47 | C |
| ATOM | 2075 | OD1 | ASN | B | 72 | 121.523 | 115.723 | 49.993 | 1.00 | 23.61 | O |
| ATOM | 2076 | ND2 | ASN | B | 72 | 122.285 | 116.614 | 51.907 | 1.00 | 21.99 | N |
| ATOM | 2077 | C | ASN | B | 72 | 122.609 | 113.137 | 49.092 | 1.00 | 23.05 | C |
| ATOM | 2078 | O | ASN | B | 72 | 123.619 | 113.514 | 48.449 | 1.00 | 22.69 | O |
| ATOM | 2079 | N | LEU | B | 73 | 121.505 | 112.674 | 48.511 | 1.00 | 23.89 | N |
| ATOM | 2080 | CA | LEU | B | 73 | 121.359 | 112.584 | 47.059 | 1.00 | 25.24 | C |
| ATOM | 2081 | CB | LEU | B | 73 | 120.038 | 111.898 | 46.722 | 1.00 | 25.68 | C |
| ATOM | 2082 | CG | LEU | B | 73 | 120.074 | 110.382 | 46.533 | 1.00 | 27.80 | C |
| ATOM | 2083 | CD1 | LEU | B | 73 | 121.157 | 109.665 | 47.310 | 1.00 | 27.77 | C |
| ATOM | 2084 | CD2 | LEU | B | 73 | 118.695 | 109.796 | 46.788 | 1.00 | 28.96 | C |
| ATOM | 2085 | C | LEU | B | 73 | 121.388 | 113.924 | 46.333 | 1.00 | 26.06 | C |
| ATOM | 2086 | O | LEU | B | 73 | 121.638 | 113.959 | 45.130 | 1.00 | 25.61 | O |
| ATOM | 2087 | N | SER | B | 74 | 121.106 | 115.011 | 47.046 | 1.00 | 26.94 | N |
| ATOM | 2088 | CA | SER | B | 74 | 121.167 | 116.350 | 46.446 | 1.00 | 28.55 | C |
| ATOM | 2089 | CB | SER | B | 74 | 120.260 | 117.308 | 47.207 | 1.00 | 28.34 | C |
| ATOM | 2090 | OG | SER | B | 74 | 120.950 | 117.891 | 48.286 | 1.00 | 30.92 | O |
| ATOM | 2091 | C | SER | B | 74 | 122.600 | 116.912 | 46.348 | 1.00 | 29.56 | C |
| ATOM | 2092 | O | SER | B | 74 | 122.811 | 117.983 | 45.766 | 1.00 | 29.88 | O |
| ATOM | 2093 | N | ARG | B | 75 | 123.582 | 116.202 | 46.898 | 1.00 | 30.55 | N |
| ATOM | 2094 | CA | ARG | B | 75 | 124.992 | 116.603 | 46.777 | 1.00 | 31.91 | C |
| ATOM | 2095 | CB | ARG | B | 75 | 125.641 | 116.735 | 48.158 | 1.00 | 31.73 | C |
| ATOM | 2096 | CG | ARG | B | 75 | 124.684 | 117.345 | 49.171 | 1.00 | 32.31 | C |
| ATOM | 2097 | CD | ARG | B | 75 | 125.137 | 118.551 | 49.959 | 1.00 | 33.23 | C |
| ATOM | 2098 | NE | ARG | B | 75 | 125.766 | 118.135 | 51.194 | 1.00 | 30.29 | N |
| ATOM | 2099 | CZ | ARG | B | 75 | 125.688 | 118.724 | 52.392 | 1.00 | 29.45 | C |
| ATOM | 2100 | NH1 | ARG | B | 75 | 124.980 | 119.823 | 52.639 | 0.50 | 28.99 | N |
| ATOM | 2101 | NH2 | ARG | B | 75 | 126.380 | 118.190 | 53.365 | 1.00 | 29.50 | N |
| ATOM | 2102 | C | ARG | B | 75 | 125.748 | 115.608 | 45.935 | 1.00 | 32.90 | C |
| ATOM | 2103 | O | ARG | B | 75 | 125.392 | 114.427 | 45.878 | 1.00 | 32.14 | O |
| ATOM | 2104 | N | ARG | B | 76 | 126.778 | 116.077 | 45.234 | 1.00 | 34.41 | N |
| ATOM | 2105 | CA | ARG | B | 76 | 127.691 | 115.128 | 44.624 | 1.00 | 35.59 | C |
| ATOM | 2106 | CB | ARG | B | 76 | 128.561 | 115.655 | 43.463 | 1.00 | 36.66 | C |
| ATOM | 2107 | CG | ARG | B | 76 | 128.757 | 117.145 | 43.324 | 1.00 | 39.83 | C |
| ATOM | 2108 | CD | ARG | B | 76 | 127.995 | 117.774 | 42.144 | 1.00 | 43.53 | C |
| ATOM | 2109 | NE | ARG | B | 76 | 127.633 | 119.167 | 42.420 | 1.00 | 47.15 | N |
| ATOM | 2110 | CZ | ARG | B | 76 | 128.486 | 120.192 | 42.453 | 1.00 | 49.26 | C |
| ATOM | 2111 | NH1 | ARG | B | 76 | 129.786 | 120.012 | 42.213 | 1.00 | 51.20 | N |
| ATOM | 2112 | NH2 | ARG | B | 76 | 128.032 | 121.416 | 42.726 | 1.00 | 48.98 | N |
| ATOM | 2113 | C | ARG | B | 76 | 128.513 | 114.699 | 45.811 | 1.00 | 34.96 | C |
| ATOM | 2114 | O | ARG | B | 76 | 128.914 | 115.515 | 46.664 | 1.00 | 37.22 | O |
| ATOM | 2115 | N | GLU | B | 77 | 128.699 | 113.407 | 45.909 | 1.00 | 33.67 | N |
| ATOM | 2116 | CA | GLU | B | 77 | 129.352 | 112.850 | 47.059 | 1.00 | 32.74 | C |
| ATOM | 2117 | CB | GLU | B | 77 | 128.369 | 112.028 | 47.890 | 1.00 | 32.43 | C |
| ATOM | 2118 | CG | GLU | B | 77 | 127.406 | 112.882 | 48.701 | 1.00 | 29.63 | C |
| ATOM | 2119 | CD | GLU | B | 77 | 126.342 | 112.063 | 49.417 | 1.00 | 28.67 | C |
| ATOM | 2120 | OE1 | GLU | B | 77 | 126.070 | 112.365 | 50.585 | 1.00 | 23.57 | O |
| ATOM | 2121 | OE2 | GLU | B | 77 | 125.776 | 111.129 | 48.797 | 1.00 | 24.61 | O |
| ATOM | 2122 | C | GLU | B | 77 | 130.457 | 111.989 | 46.514 | 1.00 | 32.88 | C |
| ATOM | 2123 | O | GLU | B | 77 | 130.184 | 110.981 | 45.880 | 1.00 | 30.70 | O |
| ATOM | 2124 | N | PRO | B | 78 | 131.705 | 112.381 | 46.783 | 1.00 | 33.65 | N |
| ATOM | 2125 | CA | PRO | B | 78 | 132.847 | 111.607 | 46.309 | 1.00 | 33.92 | C |
| ATOM | 2126 | CB | PRO | B | 78 | 134.064 | 112.428 | 46.799 | 1.00 | 34.32 | C |
| ATOM | 2127 | CG | PRO | B | 78 | 133.549 | 113.211 | 47.969 | 1.00 | 34.93 | C |
| ATOM | 2128 | CD | PRO | B | 78 | 132.132 | 113.530 | 47.607 | 1.00 | 33.85 | C |
| ATOM | 2129 | C | PRO | B | 78 | 132.846 | 110.224 | 46.926 | 1.00 | 33.33 | C |
| ATOM | 2130 | O | PRO | B | 78 | 133.538 | 109.385 | 46.393 | 1.00 | 33.75 | O |
| ATOM | 2131 | N | THR | B | 79 | 132.096 | 109.988 | 48.010 | 1.00 | 33.38 | N |
| ATOM | 2132 | CA | THR | B | 79 | 132.030 | 108.645 | 48.612 | 1.00 | 33.69 | C |
| ATOM | 2133 | CB | THR | B | 79 | 131.334 | 108.664 | 50.017 | 1.00 | 34.32 | C |
| ATOM | 2134 | OG1 | THR | B | 79 | 129.989 | 109.184 | 49.916 | 1.00 | 34.40 | O |
| ATOM | 2135 | CG2 | THR | B | 79 | 132.077 | 109.588 | 51.014 | 1.00 | 35.79 | C |
| ATOM | 2136 | C | THR | B | 79 | 131.284 | 107.602 | 47.759 | 1.00 | 32.55 | C |
| ATOM | 2137 | O | THR | B | 79 | 131.150 | 106.453 | 48.178 | 1.00 | 33.28 | O |
| ATOM | 2138 | N | ARG | B | 80 | 130.779 | 107.990 | 46.591 | 1.00 | 31.35 | N |
| ATOM | 2139 | CA | ARG | B | 80 | 129.910 | 107.090 | 45.831 | 1.00 | 29.25 | C |
| ATOM | 2140 | CB | ARG | B | 80 | 128.892 | 107.885 | 45.031 | 1.00 | 29.52 | C |
| ATOM | 2141 | CG | ARG | B | 80 | 127.934 | 108.594 | 45.870 | 1.00 | 30.24 | C |
| ATOM | 2142 | CD | ARG | B | 80 | 126.826 | 109.163 | 45.083 | 1.00 | 31.68 | C |
| ATOM | 2143 | NE | ARG | B | 80 | 126.165 | 110.233 | 45.800 | 1.00 | 32.69 | N |
| ATOM | 2144 | CZ | ARG | B | 80 | 125.075 | 110.871 | 45.388 | 1.00 | 31.43 | C |
| ATOM | 2145 | NH1 | ARG | B | 80 | 124.450 | 110.523 | 44.244 | 1.00 | 30.73 | N |
| ATOM | 2146 | NH2 | ARG | B | 80 | 124.599 | 111.851 | 46.149 | 1.00 | 26.90 | N |
| ATOM | 2147 | C | ARG | B | 80 | 130.669 | 106.208 | 44.874 | 1.00 | 27.53 | C |
| ATOM | 2148 | O | ARG | B | 80 | 131.687 | 106.586 | 44.307 | 1.00 | 26.97 | O |
| ATOM | 2149 | N | GLN | B | 81 | 130.137 | 105.017 | 44.689 | 1.00 | 24.86 | N |
| ATOM | 2150 | CA | GLN | B | 81 | 130.644 | 104.083 | 43.716 | 1.00 | 22.91 | C |
| ATOM | 2151 | CB | GLN | B | 81 | 131.175 | 102.859 | 44.407 | 1.00 | 22.59 | C |
| ATOM | 2152 | CG | GLN | B | 81 | 132.429 | 103.105 | 45.236 | 1.00 | 22.84 | C |
| ATOM | 2153 | CD | GLN | B | 81 | 132.894 | 101.845 | 45.900 | 1.00 | 23.04 | C |
| ATOM | 2154 | OE1 | GLN | B | 81 | 132.950 | 100.782 | 45.262 | 1.00 | 25.38 | O |
| ATOM | 2155 | NE2 | GLN | B | 81 | 133.212 | 101.937 | 47.191 | 1.00 | 24.23 | N |
| ATOM | 2156 | C | GLN | B | 81 | 129.436 | 103.707 | 42.886 | 1.00 | 22.10 | C |
| ATOM | 2157 | O | GLN | B | 81 | 128.417 | 103.295 | 43.461 | 1.00 | 18.52 | O |
| ATOM | 2158 | N | VAL | B | 82 | 129.568 | 103.852 | 41.559 | 1.00 | 21.47 | N |
| ATOM | 2159 | CA | VAL | B | 82 | 128.451 | 103.693 | 40.633 | 1.00 | 21.18 | C |
| ATOM | 2160 | CB | VAL | B | 82 | 128.232 | 104.975 | 39.787 | 1.00 | 21.43 | C |
| ATOM | 2161 | CG1 | VAL | B | 82 | 127.189 | 104.747 | 38.689 | 1.00 | 22.94 | C |
| ATOM | 2162 | CG2 | VAL | B | 82 | 127.821 | 106.139 | 40.677 | 1.00 | 22.47 | C |
| ATOM | 2163 | C | VAL | B | 82 | 128.693 | 102.509 | 39.727 | 1.00 | 20.83 | C |
| ATOM | 2164 | O | VAL | B | 82 | 129.747 | 102.397 | 39.099 | 1.00 | 19.83 | O |
| ATOM | 2165 | N | PHE | B | 83 | 127.706 | 101.604 | 39.667 | 1.00 | 19.08 | N |
| ATOM | 2166 | CA | PHE | B | 83 | 127.782 | 100.429 | 38.836 | 1.00 | 18.56 | C |
| ATOM | 2167 | CB | PHE | B | 83 | 127.895 | 99.193 | 39.716 | 1.00 | 18.64 | C |
| ATOM | 2168 | CG | PHE | B | 83 | 129.204 | 99.109 | 40.470 | 1.00 | 18.78 | C |
| ATOM | 2169 | CD1 | PHE | B | 83 | 129.308 | 99.606 | 41.756 | 1.00 | 18.87 | C |
| ATOM | 2170 | CE1 | PHE | B | 83 | 130.501 | 99.529 | 42.445 | 1.00 | 18.23 | C |
| ATOM | 2171 | CZ | PHE | B | 83 | 131.601 | 98.962 | 41.844 | 1.00 | 18.43 | C |
| ATOM | 2172 | CE2 | PHE | B | 83 | 131.526 | 98.493 | 40.573 | 1.00 | 21.05 | C |
| ATOM | 2173 | CD2 | PHE | B | 83 | 130.314 | 98.558 | 39.881 | 1.00 | 18.79 | C |
| ATOM | 2174 | C | PHE | B | 83 | 126.588 | 100.317 | 37.904 | 1.00 | 17.76 | C |
| ATOM | 2175 | O | PHE | B | 83 | 125.604 | 101.061 | 38.031 | 1.00 | 17.62 | O |
| ATOM | 2176 | N | ALA | B | 84 | 126.726 | 99.422 | 36.937 | 1.00 | 17.80 | N |
| ATOM | 2177 | CA | ALA | B | 84 | 125.687 | 99.110 | 35.954 | 1.00 | 17.34 | C |
| ATOM | 2178 | CB | ALA | B | 84 | 126.256 | 99.141 | 34.531 | 1.00 | 17.25 | C |
| ATOM | 2179 | C | ALA | B | 84 | 125.200 | 97.723 | 36.256 | 1.00 | 16.36 | C |
| ATOM | 2180 | O | ALA | B | 84 | 125.925 | 96.940 | 36.865 | 1.00 | 16.74 | O |
| ATOM | 2181 | N | VAL | B | 85 | 123.980 | 97.426 | 35.834 | 1.00 | 14.90 | N |
| ATOM | 2182 | CA | VAL | B | 85 | 123.438 | 96.071 | 35.938 | 1.00 | 15.10 | C |
| ATOM | 2183 | CB | VAL | B | 85 | 121.918 | 96.031 | 35.789 | 1.00 | 14.75 | C |
| ATOM | 2184 | CG1 | VAL | B | 85 | 121.404 | 94.579 | 35.890 | 1.00 | 14.05 | C |
| ATOM | 2185 | CG2 | VAL | B | 85 | 121.264 | 96.872 | 36.816 | 1.00 | 14.41 | C |
| ATOM | 2186 | C | VAL | B | 85 | 124.003 | 95.148 | 34.873 | 1.00 | 14.82 | C |
| ATOM | 2187 | O | VAL | B | 85 | 123.858 | 95.394 | 33.644 | 1.00 | 16.52 | O |
| ATOM | 2188 | N | GLN | B | 86 | 124.601 | 94.064 | 35.335 | 1.00 | 14.17 | N |
| ATOM | 2189 | CA | GLN | B | 86 | 125.126 | 93.035 | 34.464 | 1.00 | 15.51 | C |
| ATOM | 2190 | CB | GLN | B | 86 | 126.469 | 92.475 | 34.971 | 1.00 | 15.35 | C |
| ATOM | 2191 | CG | GLN | B | 86 | 127.211 | 91.620 | 33.885 | 1.00 | 17.23 | C |
| ATOM | 2192 | CD | GLN | B | 86 | 127.947 | 92.476 | 32.851 | 1.00 | 25.38 | C |
| ATOM | 2193 | OE1 | GLN | B | 86 | 127.726 | 92.345 | 31.612 | 1.00 | 26.52 | O |
| ATOM | 2194 | NE2 | GLN | B | 86 | 128.784 | 93.386 | 33.344 | 1.00 | 21.43 | N |
| ATOM | 2195 | C | GLN | B | 86 | 124.190 | 91.896 | 34.204 | 1.00 | 15.35 | C |
| ATOM | 2196 | O | GLN | B | 86 | 124.078 | 91.495 | 33.046 | 1.00 | 15.16 | O |
| ATOM | 2197 | N | ARG | B | 87 | 123.561 | 91.342 | 35.260 | 1.00 | 15.34 | N |
| ATOM | 2198 | CA | ARG | B | 87 | 122.643 | 90.229 | 35.120 | 1.00 | 14.41 | C |
| ATOM | 2199 | CB | ARG | B | 87 | 123.323 | 88.908 | 35.430 | 1.00 | 15.18 | C |
| ATOM | 2200 | CG | ARG | B | 87 | 124.474 | 88.531 | 34.513 | 1.00 | 15.39 | C |
| ATOM | 2201 | CD | ARG | B | 87 | 125.007 | 87.183 | 34.817 | 1.00 | 19.75 | C |
| ATOM | 2202 | NE | ARG | B | 87 | 125.968 | 86.766 | 33.813 | 1.00 | 23.29 | N |
| ATOM | 2203 | CZ | ARG | B | 87 | 126.522 | 85.576 | 33.741 | 1.00 | 29.29 | C |
| ATOM | 2204 | NH1 | ARG | B | 87 | 126.250 | 84.645 | 34.655 | 1.00 | 32.20 | N |
| ATOM | 2205 | NH2 | ARG | B | 87 | 127.376 | 85.317 | 32.754 | 1.00 | 29.27 | N |
| ATOM | 2206 | C | ARG | B | 87 | 121.434 | 90.370 | 36.067 | 1.00 | 14.24 | C |
| ATOM | 2207 | O | ARG | B | 87 | 121.540 | 90.974 | 37.126 | 1.00 | 13.58 | O |
| ATOM | 2208 | N | ILE | B | 88 | 120.309 | 89.758 | 35.672 | 1.00 | 13.94 | N |
| ATOM | 2209 | CA | ILE | B | 88 | 119.013 | 89.908 | 36.371 | 1.00 | 12.44 | C |
| ATOM | 2210 | CB | ILE | B | 88 | 118.011 | 90.662 | 35.479 | 1.00 | 13.71 | C |
| ATOM | 2211 | CG1 | ILE | B | 88 | 118.509 | 92.062 | 35.111 | 1.00 | 13.64 | C |
| ATOM | 2212 | CD1 | ILE | B | 88 | 117.867 | 92.657 | 33.891 | 1.00 | 17.93 | C |
| ATOM | 2213 | CG2 | ILE | B | 88 | 116.674 | 90.729 | 36.182 | 1.00 | 13.16 | C |
| ATOM | 2214 | C | ILE | B | 88 | 118.467 | 88.521 | 36.705 | 1.00 | 12.04 | C |
| ATOM | 2215 | O | ILE | B | 88 | 118.440 | 87.625 | 35.839 | 1.00 | 10.64 | O |
| ATOM | 2216 | N | PHE | B | 89 | 118.086 | 88.337 | 37.972 | 1.00 | 11.29 | N |
| ATOM | 2217 | CA | PHE | B | 89 | 117.525 | 87.080 | 38.479 | 1.00 | 11.23 | C |
| ATOM | 2218 | CB | PHE | B | 89 | 118.425 | 86.422 | 39.525 | 1.00 | 11.71 | C |
| ATOM | 2219 | CG | PHE | B | 89 | 119.835 | 86.118 | 39.025 | 1.00 | 11.74 | C |
| ATOM | 2220 | CD1 | PHE | B | 89 | 120.193 | 84.852 | 38.615 | 1.00 | 14.39 | C |
| ATOM | 2221 | CE1 | PHE | B | 89 | 121.483 | 84.598 | 38.145 | 1.00 | 15.38 | C |
| ATOM | 2222 | CZ | PHE | B | 89 | 122.421 | 85.626 | 38.090 | 1.00 | 14.36 | C |
| ATOM | 2223 | CE2 | PHE | B | 89 | 122.066 | 86.896 | 38.482 | 1.00 | 13.95 | C |
| ATOM | 2224 | CD2 | PHE | B | 89 | 120.773 | 87.132 | 38.943 | 1.00 | 14.22 | C |
| ATOM | 2225 | C | PHE | B | 89 | 116.200 | 87.441 | 39.138 | 1.00 | 11.51 | C |
| ATOM | 2226 | O | PHE | B | 89 | 116.167 | 88.276 | 40.015 | 1.00 | 11.79 | O |
| ATOM | 2227 | N | GLU | B | 90 | 115.130 | 86.814 | 38.686 | 1.00 | 12.09 | N |
| ATOM | 2228 | CA | GLU | B | 90 | 113.831 | 86.897 | 39.334 | 1.00 | 12.54 | C |
| ATOM | 2229 | CB | GLU | B | 90 | 112.888 | 87.791 | 38.511 | 1.00 | 13.55 | C |
| ATOM | 2230 | CG | GLU | B | 90 | 113.316 | 89.238 | 38.592 | 1.00 | 14.09 | C |
| ATOM | 2231 | CD | GLU | B | 90 | 112.464 | 90.205 | 37.772 | 1.00 | 16.57 | C |
| ATOM | 2232 | OE1 | GLU | B | 90 | 111.639 | 89.767 | 36.968 | 1.00 | 14.62 | O |
| ATOM | 2233 | OE2 | GLU | B | 90 | 112.649 | 91.430 | 37.939 | 1.00 | 19.16 | O |
| ATOM | 2234 | C | GLU | B | 90 | 113.253 | 85.479 | 39.449 | 1.00 | 13.03 | C |
| ATOM | 2235 | O | GLU | B | 90 | 113.439 | 84.618 | 38.557 | 1.00 | 11.18 | O |
| ATOM | 2236 | N | ASP | B | 91 | 112.500 | 85.250 | 40.524 | 1.00 | 13.86 | N |
| ATOM | 2237 | CA | ASP | B | 91 | 111.884 | 83.954 | 40.713 | 1.00 | 14.79 | C |
| ATOM | 2238 | CB | ASP | B | 91 | 112.549 | 83.240 | 41.862 | 1.00 | 14.54 | C |
| ATOM | 2239 | CG | ASP | B | 91 | 112.326 | 81.727 | 41.801 | 1.00 | 16.71 | C |
| ATOM | 2240 | OD1 | ASP | B | 91 | 111.820 | 81.172 | 42.794 | 1.00 | 25.08 | O |
| ATOM | 2241 | OD2 | ASP | B | 91 | 112.626 | 81.022 | 40.815 | 1.00 | 20.90 | O |
| ATOM | 2242 | C | ASP | B | 91 | 110.361 | 84.048 | 40.804 | 1.00 | 16.03 | C |
| ATOM | 2243 | O | ASP | B | 91 | 109.647 | 84.197 | 39.797 | 1.00 | 16.80 | O |
| ATOM | 2244 | N | GLY | B | 92 | 109.707 | 84.023 | 41.912 | 1.00 | 15.96 | N |
| ATOM | 2245 | CA | GLY | B | 92 | 108.249 | 84.125 | 41.504 | 1.00 | 18.20 | C |
| ATOM | 2246 | C | GLY | B | 92 | 107.525 | 85.421 | 40.965 | 1.00 | 16.17 | C |
| ATOM | 2247 | O | GLY | B | 92 | 106.338 | 85.480 | 41.180 | 1.00 | 15.14 | O |
| ATOM | 2248 | N | TYR | B | 94 | 108.160 | 86.466 | 40.386 | 1.00 | 14.41 | N |
| ATOM | 2249 | CA | TYR | B | 94 | 107.605 | 87.831 | 40.427 | 1.00 | 12.99 | C |
| ATOM | 2250 | CB | TYR | B | 94 | 108.555 | 88.868 | 39.818 | 1.00 | 14.05 | C |
| ATOM | 2251 | CG | TYR | B | 94 | 107.967 | 90.268 | 39.714 | 1.00 | 13.68 | C |
| ATOM | 2252 | CD1 | TYR | B | 94 | 108.061 | 90.995 | 38.531 | 1.00 | 14.98 | C |
| ATOM | 2253 | CE1 | TYR | B | 94 | 107.523 | 92.269 | 38.425 | 1.00 | 16.68 | C |
| ATOM | 2254 | CZ | TYR | B | 94 | 106.863 | 92.796 | 39.488 | 1.00 | 18.30 | C |
| ATOM | 2255 | OH | TYR | B | 94 | 106.312 | 94.019 | 39.347 | 1.00 | 23.07 | O |
| ATOM | 2256 | CE2 | TYR | B | 94 | 106.732 | 92.101 | 40.661 | 1.00 | 16.82 | C |
| ATOM | 2257 | CD2 | TYR | B | 94 | 107.290 | 90.842 | 40.775 | 1.00 | 17.03 | C |
| ATOM | 2258 | C | TYR | B | 94 | 106.241 | 87.966 | 39.746 | 1.00 | 13.20 | C |
| ATOM | 2259 | O | TYR | B | 94 | 106.114 | 87.743 | 38.570 | 1.00 | 11.28 | O |
| ATOM | 2260 | N | ASP | B | 95 | 105.252 | 88.404 | 40.513 | 1.00 | 14.15 | N |
| ATOM | 2261 | CA | ASP | B | 95 | 103.875 | 88.539 | 40.044 | 1.00 | 15.42 | C |
| ATOM | 2262 | CB | ASP | B | 95 | 102.964 | 87.847 | 41.045 | 1.00 | 15.68 | C |
| ATOM | 2263 | CG | ASP | B | 95 | 101.480 | 87.955 | 40.706 | 1.00 | 16.01 | C |
| ATOM | 2264 | OD1 | ASP | B | 95 | 101.043 | 88.937 | 40.083 | 1.00 | 16.37 | O |
| ATOM | 2265 | OD2 | ASP | B | 95 | 100.680 | 87.085 | 41.053 | 1.00 | 17.80 | O |
| ATOM | 2266 | C | ASP | B | 95 | 103.592 | 90.054 | 39.952 | 1.00 | 16.63 | C |
| ATOM | 2267 | O | ASP | B | 95 | 103.412 | 90.701 | 40.973 | 1.00 | 17.60 | O |
| ATOM | 2268 | N | PRO | B | 98 | 103.591 | 90.617 | 38.754 | 1.00 | 19.10 | N |
| ATOM | 2269 | CA | PRO | B | 98 | 103.496 | 92.076 | 38.590 | 1.00 | 21.24 | C |
| ATOM | 2270 | CB | PRO | B | 98 | 103.836 | 92.274 | 37.122 | 1.00 | 21.12 | C |
| ATOM | 2271 | CG | PRO | B | 98 | 103.355 | 91.051 | 36.505 | 1.00 | 20.21 | C |
| ATOM | 2272 | CD | PRO | B | 98 | 103.720 | 89.940 | 37.461 | 1.00 | 17.79 | C |
| ATOM | 2273 | C | PRO | B | 98 | 102.122 | 92.660 | 38.872 | 1.00 | 23.74 | C |
| ATOM | 2274 | O | PRO | B | 98 | 102.014 | 93.908 | 38.910 | 1.00 | 25.44 | O |
| ATOM | 2275 | N | VAL | B | 99 | 101.118 | 91.799 | 39.027 | 1.00 | 24.91 | N |
| ATOM | 2276 | CA | VAL | B | 99 | 99.743 | 92.188 | 39.319 | 1.00 | 27.10 | C |
| ATOM | 2277 | CB | VAL | B | 99 | 98.739 | 91.147 | 38.726 | 1.00 | 27.19 | C |
| ATOM | 2278 | CG1 | VAL | B | 99 | 97.274 | 91.545 | 39.022 | 1.00 | 29.37 | C |
| ATOM | 2279 | CG2 | VAL | B | 99 | 98.968 | 90.966 | 37.205 | 1.00 | 28.19 | C |
| ATOM | 2280 | C | VAL | B | 99 | 99.490 | 92.246 | 40.821 | 1.00 | 27.14 | C |
| ATOM | 2281 | O | VAL | B | 99 | 98.952 | 93.240 | 41.322 | 1.00 | 29.16 | O |
| ATOM | 2282 | N | ASN | B | 99A | 99.874 | 91.171 | 41.511 | 1.00 | 26.00 | N |
| ATOM | 2283 | CA | ASN | B | 99A | 99.641 | 90.982 | 42.942 | 1.00 | 25.18 | C |
| ATOM | 2284 | CB | ASN | B | 99A | 99.280 | 89.515 | 43.278 | 1.00 | 25.19 | C |
| ATOM | 2285 | CG | ASN | B | 99A | 98.176 | 88.919 | 42.377 | 1.00 | 29.51 | C |
| ATOM | 2286 | OD1 | ASN | B | 99A | 98.016 | 89.297 | 41.223 | 1.00 | 36.51 | O |
| ATOM | 2287 | ND2 | ASN | B | 99A | 97.448 | 87.940 | 42.908 | 1.00 | 31.13 | N |
| ATOM | 2288 | C | ASN | B | 99A | 100.880 | 91.322 | 43.788 | 1.00 | 23.78 | C |
| ATOM | 2289 | O | ASN | B | 99A | 100.802 | 91.243 | 44.998 | 1.00 | 24.26 | O |
| ATOM | 2290 | N | LEU | B | 99B | 102.008 | 91.676 | 43.171 | 1.00 | 21.15 | N |
| ATOM | 2291 | CA | LEU | B | 99B | 103.257 | 91.921 | 43.894 | 1.00 | 21.15 | C |
| ATOM | 2292 | CB | LEU | B | 99B | 103.280 | 93.299 | 44.593 | 1.00 | 21.26 | C |
| ATOM | 2293 | CG | LEU | B | 99B | 103.926 | 94.536 | 43.923 | 1.00 | 23.99 | C |
| ATOM | 2294 | CD1 | LEU | B | 99B | 104.086 | 94.492 | 42.405 | 1.00 | 22.91 | C |
| ATOM | 2295 | CD2 | LEU | B | 99B | 103.188 | 95.807 | 44.322 | 1.00 | 23.40 | C |
| ATOM | 2296 | C | LEU | B | 99B | 103.430 | 90.787 | 44.896 | 1.00 | 18.74 | C |
| ATOM | 2297 | O | LEU | B | 99B | 103.349 | 90.962 | 46.141 | 1.00 | 21.44 | O |
| ATOM | 2298 | N | LEU | B | 100 | 103.549 | 89.608 | 44.336 | 1.00 | 15.21 | N |
| ATOM | 2299 | CA | LEU | B | 100 | 104.083 | 88.474 | 45.031 | 1.00 | 14.59 | C |
| ATOM | 2300 | CB | LEU | B | 100 | 103.259 | 87.230 | 44.704 | 1.00 | 14.95 | C |
| ATOM | 2301 | CG | LEU | B | 100 | 101.882 | 87.201 | 45.345 | 1.00 | 18.15 | C |
| ATOM | 2302 | CD1 | LEU | B | 100 | 100.958 | 86.187 | 44.670 | 1.00 | 23.08 | C |
| ATOM | 2303 | CD2 | LEU | B | 100 | 102.057 | 86.835 | 46.800 | 1.00 | 22.71 | C |
| ATOM | 2304 | C | LEU | B | 100 | 105.520 | 88.301 | 44.560 | 1.00 | 12.86 | C |
| ATOM | 2305 | O | LEU | B | 100 | 105.830 | 88.610 | 43.415 | 1.00 | 12.09 | O |
| ATOM | 2306 | N | ASN | B | 101 | 106.372 | 87.752 | 45.430 | 1.00 | 11.90 | N |
| ATOM | 2307 | CA | ASN | B | 101 | 107.756 | 87.426 | 45.096 | 1.00 | 11.03 | C |
| ATOM | 2308 | CB | ASN | B | 101 | 107.813 | 86.221 | 44.166 | 1.00 | 10.15 | C |
| ATOM | 2309 | CG | ASN | B | 101 | 107.065 | 85.008 | 44.732 | 1.00 | 12.97 | C |
| ATOM | 2310 | OD1 | ASN | B | 101 | 106.310 | 84.342 | 44.010 | 1.00 | 22.80 | O |
| ATOM | 2311 | ND2 | ASN | B | 101 | 107.199 | 84.787 | 46.010 | 1.00 | 10.75 | N |
| ATOM | 2312 | C | ASN | B | 101 | 108.465 | 88.642 | 44.461 | 1.00 | 10.04 | C |
| ATOM | 2313 | O | ASN | B | 101 | 109.068 | 88.527 | 43.392 | 1.00 | 9.15 | O |
| ATOM | 2314 | N | ASP | B | 102 | 108.325 | 89.783 | 45.119 | 1.00 | 9.23 | N |
| ATOM | 2315 | CA | ASP | B | 102 | 108.745 | 91.067 | 44.603 | 1.00 | 9.81 | C |
| ATOM | 2316 | CB | ASP | B | 102 | 107.832 | 92.169 | 45.124 | 1.00 | 8.63 | C |
| ATOM | 2317 | CG | ASP | B | 102 | 108.062 | 93.487 | 44.454 | 1.00 | 11.95 | C |
| ATOM | 2318 | OD1 | ASP | B | 102 | 108.744 | 93.540 | 43.403 | 1.00 | 11.31 | O |
| ATOM | 2319 | OD2 | ASP | B | 102 | 107.515 | 94.530 | 44.885 | 1.00 | 8.53 | O |
| ATOM | 2320 | C | ASP | B | 102 | 110.202 | 91.295 | 44.995 | 1.00 | 8.82 | C |
| ATOM | 2321 | O | ASP | B | 102 | 110.529 | 92.140 | 45.796 | 1.00 | 9.46 | O |
| ATOM | 2322 | N | ILE | B | 103 | 111.058 | 90.471 | 44.416 | 1.00 | 9.96 | N |
| ATOM | 2323 | CA | ILE | B | 103 | 112.483 | 90.490 | 44.660 | 1.00 | 9.24 | C |
| ATOM | 2324 | CB | ILE | B | 103 | 112.864 | 89.405 | 45.730 | 1.00 | 9.61 | C |
| ATOM | 2325 | CG1 | ILE | B | 103 | 114.349 | 89.462 | 46.093 | 1.00 | 10.20 | C |
| ATOM | 2326 | CD1 | ILE | B | 103 | 114.758 | 88.586 | 47.250 | 1.00 | 10.59 | C |
| ATOM | 2327 | CG2 | ILE | B | 103 | 112.356 | 87.985 | 45.308 | 1.00 | 10.85 | C |
| ATOM | 2328 | C | ILE | B | 103 | 113.258 | 90.275 | 43.374 | 1.00 | 9.93 | C |
| ATOM | 2329 | O | ILE | B | 103 | 112.897 | 89.451 | 42.511 | 1.00 | 9.65 | O |
| ATOM | 2330 | N | VAL | B | 104 | 114.350 | 91.024 | 43.238 | 1.00 | 10.07 | N |
| ATOM | 2331 | CA | VAL | B | 104 | 115.254 | 90.842 | 42.130 | 1.00 | 9.77 | C |
| ATOM | 2332 | CB | VAL | B | 104 | 115.013 | 91.892 | 41.003 | 1.00 | 9.49 | C |
| ATOM | 2333 | CG1 | VAL | B | 104 | 115.275 | 93.310 | 41.497 | 1.00 | 8.85 | C |
| ATOM | 2334 | CG2 | VAL | B | 104 | 115.839 | 91.557 | 39.771 | 1.00 | 9.79 | C |
| ATOM | 2335 | C | VAL | B | 104 | 116.721 | 90.883 | 42.653 | 1.00 | 9.71 | C |
| ATOM | 2336 | O | VAL | B | 104 | 117.058 | 91.695 | 43.456 | 1.00 | 9.67 | O |
| ATOM | 2337 | N | ILE | B | 105 | 117.555 | 90.020 | 42.128 | 1.00 | 10.94 | N |
| ATOM | 2338 | CA | ILE | B | 105 | 119.007 | 90.067 | 42.364 | 1.00 | 12.39 | C |
| ATOM | 2339 | CB | ILE | B | 105 | 119.571 | 88.667 | 42.628 | 1.00 | 13.40 | C |
| ATOM | 2340 | CG1 | ILE | B | 105 | 118.795 | 87.887 | 43.709 | 1.00 | 15.23 | C |
| ATOM | 2341 | CD1 | ILE | B | 105 | 118.527 | 88.614 | 45.003 | 1.00 | 17.85 | C |
| ATOM | 2342 | CG2 | ILE | B | 105 | 121.020 | 88.748 | 43.021 | 1.00 | 14.98 | C |
| ATOM | 2343 | C | ILE | B | 105 | 119.636 | 90.688 | 41.123 | 1.00 | 12.13 | C |
| ATOM | 2344 | O | ILE | B | 105 | 119.388 | 90.237 | 40.013 | 1.00 | 13.07 | O |
| ATOM | 2345 | N | LEU | B | 106 | 120.352 | 91.795 | 41.289 | 1.00 | 13.12 | N |
| ATOM | 2346 | CA | LEU | B | 106 | 121.106 | 92.411 | 40.189 | 1.00 | 13.29 | C |
| ATOM | 2347 | CB | LEU | B | 106 | 120.752 | 93.882 | 40.095 | 1.00 | 13.43 | C |
| ATOM | 2348 | CG | LEU | B | 106 | 119.231 | 94.116 | 39.940 | 1.00 | 14.48 | C |
| ATOM | 2349 | CD1 | LEU | B | 106 | 118.885 | 95.536 | 40.279 | 1.00 | 14.33 | C |
| ATOM | 2350 | CD2 | LEU | B | 106 | 118.773 | 93.755 | 38.540 | 1.00 | 15.18 | C |
| ATOM | 2351 | C | LEU | B | 106 | 122.605 | 92.209 | 40.357 | 1.00 | 13.80 | C |
| ATOM | 2352 | O | LEU | B | 106 | 123.193 | 92.704 | 41.311 | 1.00 | 13.98 | O |
| ATOM | 2353 | N | GLN | B | 107 | 123.195 | 91.380 | 39.503 | 1.00 | 14.43 | N |
| ATOM | 2354 | CA | GLN | B | 107 | 124.648 | 91.325 | 39.393 | 1.00 | 14.15 | C |
| ATOM | 2355 | CB | GLN | B | 107 | 125.117 | 90.162 | 38.526 | 1.00 | 15.35 | C |
| ATOM | 2356 | CG | GLN | B | 107 | 126.642 | 89.901 | 38.636 | 1.00 | 16.62 | C |
| ATOM | 2357 | CD | GLN | B | 107 | 127.101 | 88.624 | 37.976 | 1.00 | 19.81 | C |
| ATOM | 2358 | OE1 | GLN | B | 107 | 128.299 | 88.495 | 37.625 | 1.00 | 25.52 | O |
| ATOM | 2359 | NE2 | GLN | B | 107 | 126.199 | 87.673 | 37.815 | 1.00 | 16.34 | N |
| ATOM | 2360 | C | GLN | B | 107 | 125.129 | 92.658 | 38.823 | 1.00 | 14.74 | C |
| ATOM | 2361 | O | GLN | B | 107 | 124.539 | 93.212 | 37.877 | 1.00 | 12.31 | O |
| ATOM | 2362 | N | LEU | B | 108 | 126.171 | 93.185 | 39.444 | 1.00 | 14.49 | N |
| ATOM | 2363 | CA | LEU | B | 108 | 126.847 | 94.405 | 38.996 | 1.00 | 15.34 | C |
| ATOM | 2364 | CB | LEU | B | 108 | 127.464 | 95.120 | 40.185 | 1.00 | 15.97 | C |
| ATOM | 2365 | CG | LEU | B | 108 | 126.630 | 96.104 | 41.028 | 1.00 | 18.15 | C |
| ATOM | 2366 | CD1 | LEU | B | 108 | 127.077 | 96.064 | 42.471 | 1.00 | 14.13 | C |
| ATOM | 2367 | CD2 | LEU | B | 108 | 125.106 | 96.091 | 40.879 | 1.00 | 17.45 | C |
| ATOM | 2368 | C | LEU | B | 108 | 127.947 | 94.113 | 37.967 | 1.00 | 15.46 | C |
| ATOM | 2369 | O | LEU | B | 108 | 128.469 | 93.004 | 37.885 | 1.00 | 16.16 | O |
| ATOM | 2370 | N | ASN | B | 109 | 128.283 | 95.134 | 37.193 | 1.00 | 17.12 | N |
| ATOM | 2371 | CA | ASN | B | 109 | 129.316 | 95.015 | 36.148 | 1.00 | 19.04 | C |
| ATOM | 2372 | CB | ASN | B | 109 | 129.164 | 96.127 | 35.110 | 1.00 | 19.30 | C |
| ATOM | 2373 | CG | ASN | B | 109 | 129.492 | 97.490 | 35.658 | 1.00 | 19.92 | C |
| ATOM | 2374 | OD1 | ASN | B | 109 | 129.130 | 97.817 | 36.798 | 1.00 | 18.92 | O |
| ATOM | 2375 | ND2 | ASN | B | 109 | 130.195 | 98.300 | 34.846 | 1.00 | 23.08 | N |
| ATOM | 2376 | C | ASN | B | 109 | 130.748 | 95.046 | 36.688 | 1.00 | 19.95 | C |
| ATOM | 2377 | O | ASN | B | 109 | 131.702 | 95.086 | 35.908 | 1.00 | 21.46 | O |
| ATOM | 2378 | N | GLY | B | 110 | 130.884 | 95.092 | 38.004 | 1.00 | 20.14 | N |
| ATOM | 2379 | CA | GLY | B | 110 | 132.160 | 94.888 | 38.672 | 1.00 | 20.81 | C |
| ATOM | 2380 | C | GLY | B | 110 | 131.912 | 94.811 | 40.171 | 1.00 | 20.99 | C |
| ATOM | 2381 | O | GLY | B | 110 | 130.765 | 94.723 | 40.572 | 1.00 | 20.70 | O |
| ATOM | 2382 | N | SER | B | 111 | 132.967 | 94.857 | 40.989 | 1.00 | 21.54 | N |
| ATOM | 2383 | CA | SER | B | 111 | 132.862 | 94.679 | 42.442 | 1.00 | 21.30 | C |
| ATOM | 2384 | CB | SER | B | 111 | 133.863 | 93.652 | 42.934 | 1.00 | 21.64 | C |
| ATOM | 2385 | OG | SER | B | 111 | 133.560 | 92.339 | 42.515 | 1.00 | 21.84 | O |
| ATOM | 2386 | C | SER | B | 111 | 133.124 | 95.988 | 43.173 | 1.00 | 21.25 | C |
| ATOM | 2387 | O | SER | B | 111 | 134.064 | 96.701 | 42.848 | 1.00 | 21.46 | O |
| ATOM | 2388 | N | ALA | B | 112 | 132.277 | 96.300 | 44.149 | 1.00 | 20.66 | N |
| ATOM | 2389 | CA | ALA | B | 112 | 132.486 | 97.389 | 45.067 | 1.00 | 21.19 | C |
| ATOM | 2390 | CB | ALA | B | 112 | 131.375 | 97.448 | 46.091 | 1.00 | 20.97 | C |
| ATOM | 2391 | C | ALA | B | 112 | 133.821 | 97.271 | 45.804 | 1.00 | 21.26 | C |
| ATOM | 2392 | O | ALA | B | 112 | 134.262 | 96.191 | 46.126 | 1.00 | 21.39 | O |
| ATOM | 2393 | N | THR | B | 113 | 134.413 | 98.419 | 46.080 | 1.00 | 22.20 | N |
| ATOM | 2394 | CA | THR | B | 113 | 135.482 | 98.536 | 47.064 | 1.00 | 22.79 | C |
| ATOM | 2395 | CB | THR | B | 113 | 136.342 | 99.740 | 46.738 | 1.00 | 22.45 | C |
| ATOM | 2396 | OG1 | THR | B | 113 | 136.915 | 99.527 | 45.460 | 1.00 | 23.71 | O |
| ATOM | 2397 | CG2 | THR | B | 113 | 137.570 | 99.808 | 47.656 | 1.00 | 24.82 | C |
| ATOM | 2398 | C | THR | B | 113 | 134.851 | 98.675 | 48.428 | 1.00 | 22.01 | C |
| ATOM | 2399 | O | THR | B | 113 | 134.137 | 99.640 | 48.685 | 1.00 | 21.68 | O |
| ATOM | 2400 | N | ILE | B | 114 | 135.086 | 97.677 | 49.265 | 1.00 | 22.94 | N |
| ATOM | 2401 | CA | ILE | B | 114 | 134.562 | 97.646 | 50.618 | 1.00 | 24.21 | C |
| ATOM | 2402 | CB | ILE | B | 114 | 134.638 | 96.234 | 51.209 | 1.00 | 24.56 | C |
| ATOM | 2403 | CG1 | ILE | B | 114 | 133.760 | 95.250 | 50.405 | 1.00 | 24.94 | C |
| ATOM | 2404 | CD1 | ILE | B | 114 | 132.363 | 95.792 | 50.010 | 1.00 | 24.22 | C |
| ATOM | 2405 | CG2 | ILE | B | 114 | 134.262 | 96.231 | 52.702 | 1.00 | 25.46 | C |
| ATOM | 2406 | C | ILE | B | 114 | 135.332 | 98.641 | 51.487 | 1.00 | 25.64 | C |
| ATOM | 2407 | O | ILE | B | 114 | 136.590 | 98.590 | 51.591 | 1.00 | 24.99 | O |
| ATOM | 2408 | N | ASN | B | 115 | 134.579 | 99.561 | 52.080 | 1.00 | 25.45 | N |
| ATOM | 2409 | CA | ASN | B | 115 | 135.153 | 100.533 | 53.007 | 1.00 | 26.18 | C |
| ATOM | 2410 | CB | ASN | B | 115 | 135.763 | 101.722 | 52.225 | 1.00 | 26.47 | C |
| ATOM | 2411 | CG | ASN | B | 115 | 134.772 | 102.421 | 51.311 | 1.00 | 27.28 | C |
| ATOM | 2412 | OD1 | ASN | B | 115 | 135.146 | 102.896 | 50.233 | 1.00 | 30.78 | O |
| ATOM | 2413 | ND2 | ASN | B | 115 | 133.527 | 102.521 | 51.734 | 1.00 | 24.06 | N |
| ATOM | 2414 | C | ASN | B | 115 | 134.107 | 100.959 | 54.059 | 1.00 | 26.03 | C |
| ATOM | 2415 | O | ASN | B | 115 | 133.081 | 100.293 | 54.225 | 1.00 | 24.91 | O |
| ATOM | 2416 | N | ALA | B | 116 | 134.403 | 102.022 | 54.802 | 1.00 | 25.40 | N |
| ATOM | 2417 | CA | ALA | B | 116 | 133.504 | 102.529 | 55.836 | 1.00 | 25.70 | C |
| ATOM | 2418 | CB | ALA | B | 116 | 134.023 | 103.847 | 56.379 | 1.00 | 26.33 | C |
| ATOM | 2419 | C | ALA | B | 116 | 132.072 | 102.738 | 55.324 | 1.00 | 25.05 | C |
| ATOM | 2420 | O | ALA | B | 116 | 131.117 | 102.310 | 55.968 | 1.00 | 24.86 | O |
| ATOM | 2421 | N | ASN | B | 117 | 131.947 | 103.410 | 54.181 | 1.00 | 23.64 | N |
| ATOM | 2422 | CA | ASN | B | 117 | 130.633 | 103.763 | 53.616 | 1.00 | 23.17 | C |
| ATOM | 2423 | CB | ASN | B | 117 | 130.735 | 105.060 | 52.832 | 1.00 | 23.60 | C |
| ATOM | 2424 | CG | ASN | B | 117 | 131.260 | 106.223 | 53.659 | 1.00 | 26.00 | C |
| ATOM | 2425 | OD1 | ASN | B | 117 | 131.751 | 107.204 | 53.101 | 1.00 | 31.71 | O |
| ATOM | 2426 | ND2 | ASN | B | 117 | 131.151 | 106.128 | 54.980 | 1.00 | 27.43 | N |
| ATOM | 2427 | C | ASN | B | 117 | 129.990 | 102.735 | 52.679 | 1.00 | 21.96 | C |
| ATOM | 2428 | O | ASN | B | 117 | 128.850 | 102.957 | 52.233 | 1.00 | 21.17 | O |
| ATOM | 2429 | N | VAL | B | 118 | 130.722 | 101.680 | 52.316 | 1.00 | 19.82 | N |
| ATOM | 2430 | CA | VAL | B | 118 | 130.195 | 100.657 | 51.425 | 1.00 | 20.56 | C |
| ATOM | 2431 | CB | VAL | B | 118 | 130.773 | 100.793 | 49.981 | 1.00 | 19.37 | C |
| ATOM | 2432 | CG1 | VAL | B | 118 | 130.229 | 99.685 | 49.077 | 1.00 | 19.39 | C |
| ATOM | 2433 | CG2 | VAL | B | 118 | 130.498 | 102.200 | 49.426 | 1.00 | 20.47 | C |
| ATOM | 2434 | C | VAL | B | 118 | 130.512 | 99.274 | 52.006 | 1.00 | 20.93 | C |
| ATOM | 2435 | O | VAL | B | 118 | 131.674 | 98.835 | 52.007 | 1.00 | 22.15 | O |
| ATOM | 2436 | N | GLN | B | 119 | 129.485 | 98.586 | 52.503 | 1.00 | 21.25 | N |
| ATOM | 2437 | CA | GLN | B | 119 | 129.641 | 97.243 | 53.070 | 1.00 | 21.41 | C |
| ATOM | 2438 | CB | GLN | B | 119 | 129.775 | 97.289 | 54.598 | 1.00 | 22.30 | C |
| ATOM | 2439 | CG | GLN | B | 119 | 130.870 | 98.224 | 55.038 | 1.00 | 24.31 | C |
| ATOM | 2440 | CD | GLN | B | 119 | 131.229 | 98.089 | 56.506 | 1.00 | 28.59 | C |
| ATOM | 2441 | OE1 | GLN | B | 119 | 131.202 | 99.083 | 57.252 | 1.00 | 33.10 | O |
| ATOM | 2442 | NE2 | GLN | B | 119 | 131.608 | 96.882 | 56.915 | 1.00 | 30.03 | N |
| ATOM | 2443 | C | GLN | B | 119 | 128.488 | 96.341 | 52.703 | 1.00 | 20.70 | C |
| ATOM | 2444 | O | GLN | B | 119 | 127.398 | 96.810 | 52.386 | 1.00 | 18.72 | O |
| ATOM | 2445 | N | VAL | B | 120 | 128.753 | 95.046 | 52.755 | 1.00 | 20.39 | N |
| ATOM | 2446 | CA | VAL | B | 120 | 127.774 | 94.025 | 52.389 | 1.00 | 20.30 | C |
| ATOM | 2447 | CB | VAL | B | 120 | 128.453 | 92.745 | 51.853 | 1.00 | 21.05 | C |
| ATOM | 2448 | CG1 | VAL | B | 120 | 129.309 | 93.084 | 50.634 | 1.00 | 21.25 | C |
| ATOM | 2449 | CG2 | VAL | B | 120 | 129.288 | 92.055 | 52.919 | 1.00 | 24.92 | C |
| ATOM | 2450 | C | VAL | B | 120 | 126.890 | 93.700 | 53.578 | 1.00 | 19.36 | C |
| ATOM | 2451 | O | VAL | B | 120 | 127.341 | 93.652 | 54.703 | 1.00 | 18.28 | O |
| ATOM | 2452 | N | ALA | B | 121 | 125.608 | 93.503 | 53.342 | 1.00 | 18.36 | N |
| ATOM | 2453 | CA | ALA | B | 121 | 124.673 | 93.280 | 54.449 | 1.00 | 16.97 | C |
| ATOM | 2454 | CB | ALA | B | 121 | 123.295 | 93.790 | 54.099 | 1.00 | 16.72 | C |
| ATOM | 2455 | C | ALA | B | 121 | 124.646 | 91.808 | 54.761 | 1.00 | 17.03 | C |
| ATOM | 2456 | O | ALA | B | 121 | 125.181 | 90.969 | 54.016 | 1.00 | 16.16 | O |
| ATOM | 2457 | N | GLN | B | 122 | 124.052 | 91.482 | 55.898 | 1.00 | 17.56 | N |
| ATOM | 2458 | CA | GLN | B | 122 | 123.952 | 90.082 | 56.326 | 1.00 | 18.47 | C |
| ATOM | 2459 | CB | GLN | B | 122 | 124.459 | 89.921 | 57.764 | 1.00 | 19.68 | C |
| ATOM | 2460 | CG | GLN | B | 122 | 125.993 | 90.045 | 57.872 | 1.00 | 24.15 | C |
| ATOM | 2461 | CD | GLN | B | 122 | 126.484 | 89.612 | 59.211 | 1.00 | 29.40 | C |
| ATOM | 2462 | OE1 | GLN | B | 122 | 126.303 | 90.325 | 60.188 | 1.00 | 33.87 | O |
| ATOM | 2463 | NE2 | GLN | B | 122 | 127.075 | 88.418 | 59.278 | 1.00 | 33.60 | N |
| ATOM | 2464 | C | GLN | B | 122 | 122.497 | 89.713 | 56.263 | 1.00 | 17.90 | C |
| ATOM | 2465 | O | GLN | B | 122 | 121.639 | 90.530 | 56.603 | 1.00 | 16.85 | O |
| ATOM | 2466 | N | LEU | B | 123 | 122.244 | 88.488 | 55.839 | 1.00 | 17.81 | N |
| ATOM | 2467 | CA | LEU | B | 123 | 120.920 | 87.955 | 55.656 | 1.00 | 17.90 | C |
| ATOM | 2468 | CB | LEU | B | 123 | 120.806 | 87.373 | 54.246 | 1.00 | 18.47 | C |
| ATOM | 2469 | CG | LEU | B | 123 | 120.998 | 88.325 | 53.031 | 1.00 | 20.92 | C |
| ATOM | 2470 | CD1 | LEU | B | 123 | 120.234 | 87.778 | 51.865 | 1.00 | 23.29 | C |
| ATOM | 2471 | CD2 | LEU | B | 123 | 120.611 | 89.742 | 53.295 | 1.00 | 19.05 | C |
| ATOM | 2472 | C | LEU | B | 123 | 120.573 | 86.852 | 56.684 | 1.00 | 18.15 | C |
| ATOM | 2473 | O | LEU | B | 123 | 121.464 | 86.200 | 57.247 | 1.00 | 16.66 | O |
| ATOM | 2474 | N | PRO | B | 124 | 119.270 | 86.636 | 56.916 | 1.00 | 18.04 | N |
| ATOM | 2475 | CA | PRO | B | 124 | 118.826 | 85.580 | 57.831 | 1.00 | 17.95 | C |
| ATOM | 2476 | CB | PRO | B | 124 | 117.344 | 85.890 | 58.068 | 1.00 | 18.19 | C |
| ATOM | 2477 | CG | PRO | B | 124 | 116.909 | 86.839 | 56.980 | 1.00 | 18.42 | C |
| ATOM | 2478 | CD | PRO | B | 124 | 118.136 | 87.372 | 56.326 | 1.00 | 18.69 | C |
| ATOM | 2479 | C | PRO | B | 124 | 118.984 | 84.214 | 57.210 | 1.00 | 18.00 | C |
| ATOM | 2480 | O | PRO | B | 124 | 119.367 | 84.122 | 56.032 | 1.00 | 17.33 | O |
| ATOM | 2481 | N | ALA | B | 125 | 118.713 | 83.168 | 57.998 | 1.00 | 17.69 | N |
| ATOM | 2482 | CA | ALA | B | 125 | 118.674 | 81.800 | 57.484 | 1.00 | 18.21 | C |
| ATOM | 2483 | CB | ALA | B | 125 | 118.721 | 80.817 | 58.636 | 1.00 | 18.82 | C |
| ATOM | 2484 | C | ALA | B | 125 | 117.391 | 81.587 | 56.721 | 1.00 | 17.08 | C |
| ATOM | 2485 | O | ALA | B | 125 | 116.389 | 82.221 | 57.020 | 1.00 | 17.72 | O |
| ATOM | 2486 | N | GLN | B | 126 | 117.421 | 80.645 | 55.795 | 1.00 | 16.92 | N |
| ATOM | 2487 | CA | GLN | B | 126 | 116.256 | 80.202 | 55.027 | 1.00 | 16.50 | C |
| ATOM | 2488 | CB | GLN | B | 126 | 116.636 | 79.021 | 54.115 | 1.00 | 16.70 | C |
| ATOM | 2489 | CG | GLN | B | 126 | 115.485 | 78.394 | 53.313 | 1.00 | 15.79 | C |
| ATOM | 2490 | CD | GLN | B | 126 | 114.892 | 79.351 | 52.301 | 1.00 | 19.09 | C |
| ATOM | 2491 | OE1 | GLN | B | 126 | 115.640 | 80.108 | 51.652 | 1.00 | 18.18 | O |
| ATOM | 2492 | NE2 | GLN | B | 126 | 113.562 | 79.336 | 52.157 | 1.00 | 19.15 | N |
| ATOM | 2493 | C | GLN | B | 126 | 115.116 | 79.821 | 55.967 | 1.00 | 17.57 | C |
| ATOM | 2494 | O | GLN | B | 126 | 115.335 | 79.105 | 56.955 | 1.00 | 17.02 | O |
| ATOM | 2495 | N | GLY | B | 127 | 113.937 | 80.377 | 55.698 | 1.00 | 17.58 | N |
| ATOM | 2496 | CA | GLY | B | 127 | 112.720 | 80.057 | 56.430 | 1.00 | 20.05 | C |
| ATOM | 2497 | C | GLY | B | 127 | 112.489 | 80.755 | 57.763 | 1.00 | 20.69 | C |
| ATOM | 2498 | O | GLY | B | 127 | 111.467 | 80.538 | 58.402 | 1.00 | 21.91 | O |
| ATOM | 2499 | N | ARG | B | 128 | 113.412 | 81.597 | 58.188 | 1.00 | 21.39 | N |
| ATOM | 2500 | CA | ARG | B | 128 | 113.240 | 82.352 | 59.431 | 1.00 | 22.13 | C |
| ATOM | 2501 | CB | ARG | B | 128 | 114.490 | 83.177 | 59.692 | 1.00 | 22.67 | C |
| ATOM | 2502 | CG | ARG | B | 128 | 114.488 | 83.939 | 60.966 | 1.00 | 26.93 | C |
| ATOM | 2503 | CD | ARG | B | 128 | 114.787 | 83.112 | 62.193 | 1.00 | 32.80 | C |
| ATOM | 2504 | NE | ARG | B | 128 | 113.688 | 83.088 | 63.154 | 1.00 | 37.93 | N |
| ATOM | 2505 | CZ | ARG | B | 128 | 113.413 | 84.042 | 64.050 | 1.00 | 40.02 | C |
| ATOM | 2506 | NH1 | ARG | B | 128 | 114.139 | 85.141 | 64.145 | 1.00 | 39.13 | N |
| ATOM | 2507 | NH2 | ARG | B | 128 | 112.381 | 83.883 | 64.867 | 1.00 | 42.00 | N |
| ATOM | 2508 | C | ARG | B | 128 | 112.013 | 83.256 | 59.337 | 1.00 | 22.23 | C |
| ATOM | 2509 | O | ARG | B | 128 | 111.886 | 84.017 | 58.380 | 1.00 | 22.52 | O |
| ATOM | 2510 | N | ARG | B | 129 | 111.116 | 83.163 | 60.320 | 1.00 | 22.25 | N |
| ATOM | 2511 | CA | ARG | B | 129 | 109.866 | 83.922 | 60.314 | 1.00 | 22.57 | C |
| ATOM | 2512 | CB | ARG | B | 129 | 108.641 | 82.993 | 60.185 | 1.00 | 23.76 | C |
| ATOM | 2513 | CG | ARG | B | 129 | 107.341 | 83.719 | 59.762 | 1.00 | 25.66 | C |
| ATOM | 2514 | CD | ARG | B | 129 | 106.114 | 82.797 | 59.582 | 1.00 | 29.54 | C |
| ATOM | 2515 | NE | ARG | B | 129 | 106.117 | 82.123 | 58.280 | 1.00 | 32.10 | N |
| ATOM | 2516 | CZ | ARG | B | 129 | 105.737 | 82.672 | 57.115 | 1.00 | 36.23 | C |
| ATOM | 2517 | NH1 | ARG | B | 129 | 105.267 | 83.919 | 57.055 | 1.00 | 35.60 | N |
| ATOM | 2518 | NH2 | ARG | B | 129 | 105.804 | 81.950 | 55.998 | 1.00 | 38.08 | N |
| ATOM | 2519 | C | ARG | B | 129 | 109.804 | 84.746 | 61.577 | 1.00 | 21.88 | C |
| ATOM | 2520 | O | ARG | B | 129 | 109.936 | 84.227 | 62.687 | 1.00 | 23.42 | O |
| ATOM | 2521 | N | LEU | B | 130 | 109.637 | 86.050 | 61.406 | 1.00 | 20.16 | N |
| ATOM | 2522 | CA | LEU | B | 130 | 109.594 | 86.989 | 62.509 | 1.00 | 19.60 | C |
| ATOM | 2523 | CB | LEU | B | 130 | 110.017 | 88.376 | 62.053 | 1.00 | 19.10 | C |
| ATOM | 2524 | CG | LEU | B | 130 | 111.477 | 88.513 | 61.591 | 1.00 | 19.32 | C |
| ATOM | 2525 | CD1 | LEU | B | 130 | 111.722 | 89.945 | 61.159 | 1.00 | 19.25 | C |
| ATOM | 2526 | CD2 | LEU | B | 130 | 112.480 | 88.113 | 62.692 | 1.00 | 20.31 | C |
| ATOM | 2527 | C | LEU | B | 130 | 108.200 | 87.060 | 63.083 | 1.00 | 19.11 | C |
| ATOM | 2528 | O | LEU | B | 130 | 107.218 | 87.170 | 62.355 | 1.00 | 19.16 | O |
| ATOM | 2529 | N | GLY | B | 131 | 108.130 | 87.042 | 64.398 | 1.00 | 17.89 | N |
| ATOM | 2530 | CA | GLY | B | 131 | 106.868 | 87.125 | 65.091 | 1.00 | 17.92 | C |
| ATOM | 2531 | C | GLY | B | 131 | 106.355 | 88.546 | 65.167 | 1.00 | 16.99 | C |
| ATOM | 2532 | O | GLY | B | 131 | 107.111 | 89.537 | 65.085 | 1.00 | 15.52 | O |
| ATOM | 2533 | N | ASN | B | 132 | 105.047 | 88.632 | 65.351 | 1.00 | 17.92 | N |
| ATOM | 2534 | CA | ASN | B | 132 | 104.368 | 89.865 | 65.739 | 1.00 | 18.52 | C |
| ATOM | 2535 | CB | ASN | B | 132 | 102.999 | 89.528 | 66.305 | 1.00 | 20.01 | C |
| ATOM | 2536 | CG | ASN | B | 132 | 101.894 | 89.882 | 65.403 | 1.00 | 24.20 | C |
| ATOM | 2537 | OD1 | ASN | B | 132 | 101.710 | 91.056 | 65.035 | 1.00 | 25.52 | O |
| ATOM | 2538 | ND2 | ASN | B | 132 | 101.094 | 88.874 | 65.053 | 1.00 | 30.98 | N |
| ATOM | 2539 | C | ASN | B | 132 | 105.108 | 90.576 | 66.843 | 1.00 | 17.87 | C |
| ATOM | 2540 | O | ASN | B | 132 | 105.387 | 89.980 | 67.903 | 1.00 | 17.20 | O |
| ATOM | 2541 | N | GLY | B | 133 | 105.409 | 91.838 | 66.608 | 1.00 | 16.46 | N |
| ATOM | 2542 | CA | GLY | B | 133 | 105.970 | 92.724 | 67.589 | 1.00 | 16.75 | C |
| ATOM | 2543 | C | GLY | B | 133 | 107.452 | 92.976 | 67.465 | 1.00 | 16.74 | C |
| ATOM | 2544 | O | GLY | B | 133 | 107.977 | 93.834 | 68.150 | 1.00 | 16.95 | O |
| ATOM | 2545 | N | VAL | B | 134 | 108.132 | 92.209 | 66.625 | 1.00 | 16.29 | N |
| ATOM | 2546 | CA | VAL | B | 134 | 109.561 | 92.388 | 66.447 | 1.00 | 16.91 | C |
| ATOM | 2547 | CB | VAL | B | 134 | 110.184 | 91.275 | 65.541 | 1.00 | 17.11 | C |
| ATOM | 2548 | CG1 | VAL | B | 134 | 111.627 | 91.627 | 65.126 | 1.00 | 16.70 | C |
| ATOM | 2549 | CG2 | VAL | B | 134 | 110.129 | 89.940 | 66.260 | 1.00 | 17.26 | C |
| ATOM | 2550 | C | VAL | B | 134 | 109.892 | 93.803 | 65.974 | 1.00 | 17.27 | C |
| ATOM | 2551 | O | VAL | B | 134 | 109.213 | 94.372 | 65.131 | 1.00 | 17.25 | O |
| ATOM | 2552 | N | GLN | B | 135 | 110.910 | 94.377 | 66.578 | 1.00 | 16.50 | N |
| ATOM | 2553 | CA | GLN | B | 135 | 111.299 | 95.746 | 66.334 | 1.00 | 17.53 | C |
| ATOM | 2554 | CB | GLN | B | 135 | 111.873 | 96.326 | 67.615 | 1.00 | 18.83 | C |
| ATOM | 2555 | CG | GLN | B | 135 | 110.837 | 97.051 | 68.372 | 1.00 | 23.15 | C |
| ATOM | 2556 | CD | GLN | B | 135 | 110.331 | 98.197 | 67.528 | 1.00 | 26.53 | C |
| ATOM | 2557 | OE1 | GLN | B | 135 | 111.146 | 98.900 | 66.858 | 1.00 | 35.36 | O |
| ATOM | 2558 | NE2 | GLN | B | 135 | 109.012 | 98.339 | 67.454 | 1.00 | 25.35 | N |
| ATOM | 2559 | C | GLN | B | 135 | 112.313 | 95.839 | 65.210 | 1.00 | 16.21 | C |
| ATOM | 2560 | O | GLN | B | 135 | 113.407 | 95.283 | 65.320 | 1.00 | 16.67 | O |
| ATOM | 2561 | N | CYS | B | 136 | 111.971 | 96.575 | 64.152 | 1.00 | 14.86 | N |
| ATOM | 2562 | CA | CYS | B | 136 | 112.868 | 96.707 | 62.986 | 1.00 | 13.99 | C |
| ATOM | 2563 | CB | CYS | B | 136 | 112.269 | 95.975 | 61.794 | 1.00 | 13.44 | C |
| ATOM | 2564 | SG | CYS | B | 136 | 111.905 | 94.205 | 62.012 | 1.00 | 14.07 | S |
| ATOM | 2565 | C | CYS | B | 136 | 113.061 | 98.163 | 62.640 | 1.00 | 13.92 | C |
| ATOM | 2566 | O | CYS | B | 136 | 112.430 | 99.056 | 63.225 | 1.00 | 12.73 | O |
| ATOM | 2567 | N | LEU | B | 137 | 113.897 | 98.391 | 61.634 | 1.00 | 13.90 | N |
| ATOM | 2568 | CA | LEU | B | 137 | 114.173 | 99.709 | 61.129 | 1.00 | 14.48 | C |
| ATOM | 2569 | CB | LEU | B | 137 | 115.615 | 100.074 | 61.450 | 1.00 | 14.98 | C |
| ATOM | 2570 | CG | LEU | B | 137 | 116.043 | 101.534 | 61.255 | 1.00 | 18.33 | C |
| ATOM | 2571 | CD1 | LEU | B | 137 | 115.367 | 102.470 | 62.281 | 1.00 | 17.21 | C |
| ATOM | 2572 | CD2 | LEU | B | 137 | 117.563 | 101.625 | 61.375 | 1.00 | 20.07 | C |
| ATOM | 2573 | C | LEU | B | 137 | 113.994 | 99.641 | 59.618 | 1.00 | 14.01 | C |
| ATOM | 2574 | O | LEU | B | 137 | 114.617 | 98.814 | 58.970 | 1.00 | 14.65 | O |
| ATOM | 2575 | N | ALA | B | 138 | 113.136 | 100.494 | 59.087 | 1.00 | 13.71 | N |
| ATOM | 2576 | CA | ALA | B | 138 | 112.999 | 100.704 | 57.668 | 1.00 | 12.85 | C |
| ATOM | 2577 | CB | ALA | B | 138 | 111.525 | 100.901 | 57.295 | 1.00 | 12.51 | C |
| ATOM | 2578 | C | ALA | B | 138 | 113.780 | 101.958 | 57.323 | 1.00 | 13.21 | C |
| ATOM | 2579 | O | ALA | B | 138 | 114.085 | 102.771 | 58.193 | 1.00 | 13.08 | O |
| ATOM | 2580 | N | MET | B | 139 | 114.072 | 102.097 | 56.041 | 1.00 | 12.35 | N |
| ATOM | 2581 | CA | MET | B | 139 | 114.822 | 103.217 | 55.535 | 1.00 | 13.78 | C |
| ATOM | 2582 | CB | MET | B | 139 | 116.316 | 102.983 | 55.810 | 1.00 | 12.92 | C |
| ATOM | 2583 | CG | MET | B | 139 | 116.881 | 101.763 | 55.095 | 1.00 | 14.56 | C |
| ATOM | 2584 | SD | MET | B | 139 | 118.658 | 101.506 | 55.566 | 1.00 | 14.58 | S |
| ATOM | 2585 | CE | MET | B | 139 | 118.447 | 100.939 | 57.222 | 1.00 | 14.99 | C |
| ATOM | 2586 | C | MET | B | 139 | 114.583 | 103.442 | 54.039 | 1.00 | 13.08 | C |
| ATOM | 2587 | O | MET | B | 139 | 114.179 | 102.532 | 53.324 | 1.00 | 13.31 | O |
| ATOM | 2588 | N | GLY | B | 140 | 114.883 | 104.643 | 53.574 | 1.00 | 13.95 | N |
| ATOM | 2589 | CA | GLY | B | 140 | 114.827 | 104.973 | 52.159 | 1.00 | 14.15 | C |
| ATOM | 2590 | C | GLY | B | 140 | 114.882 | 106.478 | 51.849 | 1.00 | 13.96 | C |
| ATOM | 2591 | O | GLY | B | 140 | 114.883 | 107.346 | 52.750 | 1.00 | 13.80 | O |
| ATOM | 2592 | N | TRP | B | 141 | 114.973 | 106.756 | 50.552 | 1.00 | 14.07 | N |
| ATOM | 2593 | CA | TRP | B | 141 | 115.053 | 108.102 | 50.002 | 1.00 | 14.44 | C |
| ATOM | 2594 | CB | TRP | B | 141 | 116.198 | 108.194 | 48.999 | 1.00 | 14.33 | C |
| ATOM | 2595 | CG | TRP | B | 141 | 117.596 | 108.351 | 49.566 | 1.00 | 13.99 | C |
| ATOM | 2596 | CD1 | TRP | B | 141 | 118.114 | 109.472 | 50.150 | 1.00 | 15.60 | C |
| ATOM | 2597 | NE1 | TRP | B | 141 | 119.426 | 109.266 | 50.497 | 1.00 | 15.25 | N |
| ATOM | 2598 | CE2 | TRP | B | 141 | 119.787 | 107.991 | 50.152 | 1.00 | 15.82 | C |
| ATOM | 2599 | CD2 | TRP | B | 141 | 118.666 | 107.394 | 49.521 | 1.00 | 13.93 | C |
| ATOM | 2600 | CE3 | TRP | B | 141 | 118.784 | 106.081 | 49.062 | 1.00 | 13.34 | C |
| ATOM | 2601 | CZ3 | TRP | B | 141 | 119.992 | 105.425 | 49.201 | 1.00 | 13.86 | C |
| ATOM | 2602 | CH2 | TRP | B | 141 | 121.080 | 106.055 | 49.802 | 1.00 | 15.30 | C |
| ATOM | 2603 | CZ2 | TRP | B | 141 | 120.990 | 107.349 | 50.280 | 1.00 | 13.89 | C |
| ATOM | 2604 | C | TRP | B | 141 | 113.722 | 108.462 | 49.301 | 1.00 | 14.29 | C |
| ATOM | 2605 | O | TRP | B | 141 | 113.698 | 109.327 | 48.435 | 1.00 | 16.32 | O |
| ATOM | 2606 | N | GLY | B | 142 | 112.625 | 107.800 | 49.671 | 1.00 | 14.30 | N |
| ATOM | 2607 | CA | GLY | B | 142 | 111.315 | 108.046 | 49.082 | 1.00 | 13.79 | C |
| ATOM | 2608 | C | GLY | B | 142 | 110.611 | 109.243 | 49.682 | 1.00 | 14.12 | C |
| ATOM | 2609 | O | GLY | B | 142 | 111.181 | 109.975 | 50.503 | 1.00 | 13.65 | O |
| ATOM | 2610 | N | LEU | B | 143 | 109.325 | 109.385 | 49.347 | 1.00 | 15.55 | N |
| ATOM | 2611 | CA | LEU | B | 143 | 108.538 | 110.556 | 49.695 | 1.00 | 16.01 | C |
| ATOM | 2612 | CB | LEU | B | 143 | 107.126 | 110.473 | 49.071 | 1.00 | 16.53 | C |
| ATOM | 2613 | CG | LEU | B | 143 | 107.026 | 110.491 | 47.528 | 1.00 | 16.15 | C |
| ATOM | 2614 | CD1 | LEU | B | 143 | 105.528 | 110.564 | 47.081 | 1.00 | 18.70 | C |
| ATOM | 2615 | CD2 | LEU | B | 143 | 107.785 | 111.644 | 46.953 | 1.00 | 18.04 | C |
| ATOM | 2616 | C | LEU | B | 143 | 108.412 | 110.679 | 51.203 | 1.00 | 17.65 | C |
| ATOM | 2617 | O | LEU | B | 143 | 108.285 | 109.677 | 51.899 | 1.00 | 15.49 | O |
| ATOM | 2618 | N | LEU | B | 144 | 108.405 | 111.932 | 51.666 | 1.00 | 19.06 | N |
| ATOM | 2619 | CA | LEU | B | 144 | 108.293 | 112.322 | 53.070 | 1.00 | 20.80 | C |
| ATOM | 2620 | CB | LEU | B | 144 | 109.058 | 113.639 | 53.269 | 1.00 | 21.21 | C |
| ATOM | 2621 | CG | LEU | B | 144 | 110.566 | 113.540 | 53.016 | 1.00 | 21.59 | C |
| ATOM | 2622 | CD1 | LEU | B | 144 | 111.200 | 114.914 | 53.017 | 1.00 | 22.01 | C |
| ATOM | 2623 | CD2 | LEU | B | 144 | 111.247 | 112.625 | 54.022 | 1.00 | 22.80 | C |
| ATOM | 2624 | C | LEU | B | 144 | 106.859 | 112.498 | 53.553 | 1.00 | 22.72 | C |
| ATOM | 2625 | O | LEU | B | 144 | 106.620 | 112.643 | 54.755 | 1.00 | 23.50 | O |
| ATOM | 2626 | N | GLY | B | 145 | 105.905 | 112.502 | 52.617 | 1.00 | 25.54 | N |
| ATOM | 2627 | CA | GLY | B | 145 | 104.482 | 112.447 | 52.938 | 1.00 | 27.38 | C |
| ATOM | 2628 | C | GLY | B | 145 | 103.709 | 113.663 | 52.447 | 1.00 | 29.41 | C |
| ATOM | 2629 | O | GLY | B | 145 | 104.278 | 114.515 | 51.803 | 1.00 | 29.67 | O |
| ATOM | 2630 | N | ARG | B | 147 | 102.418 | 113.732 | 52.781 | 1.00 | 32.24 | N |
| ATOM | 2631 | CA | ARG | B | 147 | 101.501 | 114.815 | 52.375 | 1.00 | 34.21 | C |
| ATOM | 2632 | CB | ARG | B | 147 | 100.218 | 114.706 | 53.218 | 1.00 | 34.87 | C |
| ATOM | 2633 | CG | ARG | B | 147 | 99.096 | 115.654 | 52.850 | 1.00 | 36.67 | C |
| ATOM | 2634 | CD | ARG | B | 147 | 97.720 | 115.203 | 53.351 | 1.00 | 39.15 | C |
| ATOM | 2635 | NE | ARG | B | 147 | 96.705 | 115.566 | 52.360 | 1.00 | 42.55 | N |
| ATOM | 2636 | CZ | ARG | B | 147 | 95.939 | 114.715 | 51.667 | 1.00 | 44.36 | C |
| ATOM | 2637 | NH1 | ARG | B | 147 | 95.998 | 113.391 | 51.854 | 1.00 | 43.69 | N |
| ATOM | 2638 | NH2 | ARG | B | 147 | 95.075 | 115.212 | 50.783 | 1.00 | 45.87 | N |
| ATOM | 2639 | C | ARG | B | 147 | 102.125 | 116.231 | 52.461 | 1.00 | 35.32 | C |
| ATOM | 2640 | O | ARG | B | 147 | 102.501 | 116.712 | 53.537 | 1.00 | 35.06 | O |
| ATOM | 2641 | N | ASN | B | 148 | 102.270 | 116.859 | 51.294 | 1.00 | 37.48 | N |
| ATOM | 2642 | CA | ASN | B | 148 | 102.913 | 118.180 | 51.133 | 1.00 | 39.21 | C |
| ATOM | 2643 | CB | ASN | B | 148 | 101.900 | 119.296 | 51.396 | 1.00 | 39.57 | C |
| ATOM | 2644 | CG | ASN | B | 148 | 100.816 | 119.369 | 50.296 | 1.00 | 41.96 | C |
| ATOM | 2645 | OD1 | ASN | B | 148 | 101.071 | 119.844 | 49.177 | 1.00 | 44.60 | O |
| ATOM | 2646 | ND2 | ASN | B | 148 | 99.622 | 118.870 | 50.605 | 1.00 | 43.16 | N |
| ATOM | 2647 | C | ASN | B | 148 | 104.272 | 118.380 | 51.878 | 1.00 | 40.00 | C |
| ATOM | 2648 | O | ASN | B | 148 | 104.648 | 119.486 | 52.294 | 1.00 | 39.96 | O |
| ATOM | 2649 | N | ARG | B | 149 | 105.009 | 117.281 | 52.019 | 1.00 | 40.30 | N |
| ATOM | 2650 | CA | ARG | B | 149 | 106.441 | 117.333 | 52.263 | 1.00 | 40.68 | C |
| ATOM | 2651 | CB | ARG | B | 149 | 106.811 | 116.446 | 53.443 | 1.00 | 41.37 | C |
| ATOM | 2652 | CG | ARG | B | 149 | 106.035 | 116.789 | 54.729 | 1.00 | 44.56 | C |
| ATOM | 2653 | CD | ARG | B | 149 | 106.695 | 116.340 | 56.042 | 1.00 | 48.34 | C |
| ATOM | 2654 | NE | ARG | B | 149 | 108.143 | 116.616 | 56.072 | 1.00 | 52.08 | N |
| ATOM | 2655 | CZ | ARG | B | 149 | 108.996 | 116.146 | 56.991 | 1.00 | 54.25 | C |
| ATOM | 2656 | NH1 | ARG | B | 149 | 108.568 | 115.388 | 57.997 | 1.00 | 55.73 | N |
| ATOM | 2657 | NH2 | ARG | B | 149 | 110.291 | 116.449 | 56.908 | 1.00 | 55.62 | N |
| ATOM | 2658 | C | ARG | B | 149 | 107.090 | 116.881 | 50.947 | 1.00 | 39.89 | C |
| ATOM | 2659 | O | ARG | B | 149 | 106.411 | 116.396 | 50.031 | 1.00 | 40.81 | O |
| ATOM | 2660 | N | GLY | B | 150 | 108.385 | 117.058 | 50.796 | 1.00 | 38.79 | N |
| ATOM | 2661 | CA | GLY | B | 150 | 108.964 | 116.787 | 49.490 | 1.00 | 37.52 | C |
| ATOM | 2662 | C | GLY | B | 150 | 109.295 | 115.321 | 49.323 | 1.00 | 36.21 | C |
| ATOM | 2663 | O | GLY | B | 150 | 108.606 | 114.442 | 49.827 | 1.00 | 35.09 | O |
| ATOM | 2664 | N | ILE | B | 151 | 110.362 | 115.074 | 48.576 | 1.00 | 34.67 | N |
| ATOM | 2665 | CA | ILE | B | 151 | 111.087 | 113.824 | 48.661 | 1.00 | 33.27 | C |
| ATOM | 2666 | CB | ILE | B | 151 | 111.426 | 113.325 | 47.256 | 1.00 | 33.48 | C |
| ATOM | 2667 | CG1 | ILE | B | 151 | 111.722 | 111.824 | 47.270 | 1.00 | 33.98 | C |
| ATOM | 2668 | CD1 | ILE | B | 151 | 111.911 | 111.252 | 45.889 | 1.00 | 33.96 | C |
| ATOM | 2669 | CG2 | ILE | B | 151 | 112.613 | 114.104 | 46.657 | 1.00 | 33.95 | C |
| ATOM | 2670 | C | ILE | B | 151 | 112.350 | 114.065 | 49.514 | 1.00 | 31.24 | C |
| ATOM | 2671 | O | ILE | B | 151 | 112.746 | 115.217 | 49.777 | 1.00 | 30.43 | O |
| ATOM | 2672 | N | ALA | B | 152 | 112.963 | 112.978 | 49.958 | 1.00 | 29.14 | N |
| ATOM | 2673 | CA | ALA | B | 152 | 114.129 | 113.047 | 50.808 | 1.00 | 27.86 | C |
| ATOM | 2674 | CB | ALA | B | 152 | 114.349 | 111.705 | 51.462 | 1.00 | 27.75 | C |
| ATOM | 2675 | C | ALA | B | 152 | 115.374 | 113.449 | 50.013 | 1.00 | 26.75 | C |
| ATOM | 2676 | O | ALA | B | 152 | 115.622 | 112.934 | 48.937 | 1.00 | 27.40 | O |
| ATOM | 2677 | N | SER | B | 153 | 116.180 | 114.348 | 50.546 | 1.00 | 25.28 | N |
| ATOM | 2678 | CA | SER | B | 153 | 117.553 | 114.458 | 50.043 | 1.00 | 25.13 | C |
| ATOM | 2679 | CB | SER | B | 153 | 118.083 | 115.895 | 50.169 | 1.00 | 24.80 | C |
| ATOM | 2680 | OG | SER | B | 153 | 117.155 | 116.799 | 49.593 | 1.00 | 27.23 | O |
| ATOM | 2681 | C | SER | B | 153 | 118.419 | 113.486 | 50.832 | 1.00 | 23.18 | C |
| ATOM | 2682 | O | SER | B | 153 | 119.153 | 112.685 | 50.279 | 1.00 | 23.62 | O |
| ATOM | 2683 | N | VAL | B | 154 | 118.336 | 113.591 | 52.147 | 1.00 | 22.53 | N |
| ATOM | 2684 | CA | VAL | B | 154 | 119.097 | 112.739 | 53.037 | 1.00 | 22.08 | C |
| ATOM | 2685 | CB | VAL | B | 154 | 119.417 | 113.444 | 54.362 | 1.00 | 22.02 | C |
| ATOM | 2686 | CG1 | VAL | B | 154 | 118.173 | 113.879 | 55.022 | 1.00 | 25.96 | C |
| ATOM | 2687 | CG2 | VAL | B | 154 | 120.184 | 112.521 | 55.308 | 1.00 | 23.69 | C |
| ATOM | 2688 | C | VAL | B | 154 | 118.341 | 111.458 | 53.352 | 1.00 | 20.58 | C |
| ATOM | 2689 | O | VAL | B | 154 | 117.142 | 111.491 | 53.617 | 1.00 | 20.99 | O |
| ATOM | 2690 | N | LEU | B | 155 | 119.069 | 110.349 | 53.358 | 1.00 | 19.69 | N |
| ATOM | 2691 | CA | LEU | B | 155 | 118.533 | 109.041 | 53.725 | 1.00 | 18.42 | C |
| ATOM | 2692 | CB | LEU | B | 155 | 119.651 | 108.022 | 53.846 | 1.00 | 17.95 | C |
| ATOM | 2693 | CG | LEU | B | 155 | 119.286 | 106.601 | 54.305 | 1.00 | 19.19 | C |
| ATOM | 2694 | CD1 | LEU | B | 155 | 118.336 | 105.962 | 53.303 | 1.00 | 18.81 | C |
| ATOM | 2695 | CD2 | LEU | B | 155 | 120.512 | 105.728 | 54.497 | 1.00 | 19.75 | C |
| ATOM | 2696 | C | LEU | B | 155 | 117.724 | 109.088 | 55.033 | 1.00 | 17.89 | C |
| ATOM | 2697 | O | LEU | B | 155 | 118.171 | 109.607 | 56.041 | 1.00 | 18.61 | O |
| ATOM | 2698 | N | GLN | B | 156 | 116.519 | 108.525 | 55.007 | 1.00 | 16.32 | N |
| ATOM | 2699 | CA | GLN | B | 156 | 115.639 | 108.537 | 56.167 | 1.00 | 15.35 | C |
| ATOM | 2700 | CB | GLN | B | 156 | 114.223 | 109.010 | 55.776 | 1.00 | 15.37 | C |
| ATOM | 2701 | CG | GLN | B | 156 | 114.161 | 110.298 | 55.031 | 1.00 | 15.47 | C |
| ATOM | 2702 | CD | GLN | B | 156 | 114.343 | 111.530 | 55.923 | 1.00 | 16.31 | C |
| ATOM | 2703 | OE1 | GLN | B | 156 | 115.254 | 112.363 | 55.682 | 1.00 | 19.82 | O |
| ATOM | 2704 | NE2 | GLN | B | 156 | 113.529 | 111.633 | 56.949 | 1.00 | 13.47 | N |
| ATOM | 2705 | C | GLN | B | 156 | 115.566 | 107.141 | 56.774 | 1.00 | 13.84 | C |
| ATOM | 2706 | O | GLN | B | 156 | 115.724 | 106.138 | 56.073 | 1.00 | 14.36 | O |
| ATOM | 2707 | N | GLU | B | 157 | 115.336 | 107.069 | 58.080 | 1.00 | 13.76 | N |
| ATOM | 2708 | CA | GLU | B | 157 | 115.091 | 105.798 | 58.756 | 1.00 | 13.36 | C |
| ATOM | 2709 | CB | GLU | B | 157 | 116.370 | 105.286 | 59.491 | 1.00 | 13.55 | C |
| ATOM | 2710 | CG | GLU | B | 157 | 116.684 | 106.081 | 60.739 | 1.00 | 16.46 | C |
| ATOM | 2711 | CD | GLU | B | 157 | 118.000 | 105.676 | 61.389 | 1.00 | 16.62 | C |
| ATOM | 2712 | OE1 | GLU | B | 157 | 119.053 | 105.663 | 60.722 | 1.00 | 16.45 | O |
| ATOM | 2713 | OE2 | GLU | B | 157 | 117.954 | 105.369 | 62.585 | 1.00 | 18.14 | O |
| ATOM | 2714 | C | GLU | B | 157 | 113.893 | 105.905 | 59.703 | 1.00 | 13.08 | C |
| ATOM | 2715 | O | GLU | B | 157 | 113.466 | 106.978 | 60.089 | 1.00 | 12.86 | O |
| ATOM | 2716 | N | LEU | B | 158 | 113.309 | 104.770 | 60.044 | 1.00 | 14.26 | N |
| ATOM | 2717 | CA | LEU | B | 158 | 112.059 | 104.783 | 60.794 | 1.00 | 13.96 | C |
| ATOM | 2718 | CB | LEU | B | 158 | 110.869 | 104.939 | 59.828 | 1.00 | 14.38 | C |
| ATOM | 2719 | CG | LEU | B | 158 | 109.449 | 104.697 | 60.340 | 1.00 | 14.96 | C |
| ATOM | 2720 | CD1 | LEU | B | 158 | 109.030 | 105.865 | 61.259 | 1.00 | 15.23 | C |
| ATOM | 2721 | CD2 | LEU | B | 158 | 108.442 | 104.482 | 59.182 | 1.00 | 15.47 | C |
| ATOM | 2722 | C | LEU | B | 158 | 111.946 | 103.490 | 61.591 | 1.00 | 13.72 | C |
| ATOM | 2723 | O | LEU | B | 158 | 112.094 | 102.414 | 61.039 | 1.00 | 14.07 | O |
| ATOM | 2724 | N | ASN | B | 159 | 111.708 | 103.595 | 62.899 | 1.00 | 14.11 | N |
| ATOM | 2725 | CA | ASN | B | 159 | 111.422 | 102.422 | 63.728 | 1.00 | 13.71 | C |
| ATOM | 2726 | CB | ASN | B | 159 | 111.518 | 102.757 | 65.208 | 1.00 | 14.33 | C |
| ATOM | 2727 | CG | ASN | B | 159 | 112.937 | 102.954 | 65.655 | 1.00 | 13.77 | C |
| ATOM | 2728 | OD1 | ASN | B | 159 | 113.616 | 103.822 | 65.134 | 1.00 | 16.45 | O |
| ATOM | 2729 | ND2 | ASN | B | 159 | 113.380 | 102.122 | 66.561 | 1.00 | 13.42 | N |
| ATOM | 2730 | C | ASN | B | 159 | 110.021 | 101.912 | 63.436 | 1.00 | 13.46 | C |
| ATOM | 2731 | O | ASN | B | 159 | 109.059 | 102.688 | 63.400 | 1.00 | 13.82 | O |
| ATOM | 2732 | N | VAL | B | 160 | 109.925 | 100.598 | 63.302 | 1.00 | 14.33 | N |
| ATOM | 2733 | CA | VAL | B | 160 | 108.745 | 99.933 | 62.789 | 1.00 | 14.34 | C |
| ATOM | 2734 | CB | VAL | B | 160 | 109.070 | 99.804 | 61.258 | 1.00 | 15.97 | C |
| ATOM | 2735 | CG1 | VAL | B | 160 | 109.338 | 98.400 | 60.175 | 1.00 | 12.59 | C |
| ATOM | 2736 | CG2 | VAL | B | 160 | 108.266 | 100.778 | 60.415 | 1.00 | 17.18 | C |
| ATOM | 2737 | C | VAL | B | 160 | 108.576 | 98.604 | 63.514 | 1.00 | 14.93 | C |
| ATOM | 2738 | O | VAL | B | 160 | 109.580 | 98.008 | 63.958 | 1.00 | 14.65 | O |
| ATOM | 2739 | N | THR | B | 162 | 107.333 | 98.108 | 63.606 | 1.00 | 13.51 | N |
| ATOM | 2740 | CA | THR | B | 162 | 107.036 | 96.834 | 64.272 | 1.00 | 14.65 | C |
| ATOM | 2741 | CB | THR | B | 162 | 106.026 | 97.114 | 65.433 | 1.00 | 15.13 | C |
| ATOM | 2742 | OG1 | THR | B | 162 | 106.680 | 97.934 | 66.412 | 1.00 | 22.20 | O |
| ATOM | 2743 | CG2 | THR | B | 162 | 105.771 | 95.898 | 66.199 | 1.00 | 18.59 | C |
| ATOM | 2744 | C | THR | B | 162 | 106.458 | 95.792 | 63.304 | 1.00 | 12.95 | C |
| ATOM | 2745 | O | THR | B | 162 | 105.541 | 96.115 | 62.521 | 1.00 | 10.73 | O |
| ATOM | 2746 | N | VAL | B | 163 | 106.964 | 94.552 | 63.361 | 1.00 | 11.22 | N |
| ATOM | 2747 | CA | VAL | B | 163 | 106.416 | 93.466 | 62.569 | 1.00 | 11.12 | C |
| ATOM | 2748 | CB | VAL | B | 163 | 107.287 | 92.189 | 62.664 | 1.00 | 10.25 | C |
| ATOM | 2749 | CG1 | VAL | B | 163 | 106.624 | 91.069 | 62.029 | 1.00 | 11.72 | C |
| ATOM | 2750 | CG2 | VAL | B | 163 | 108.609 | 92.424 | 62.042 | 1.00 | 11.69 | C |
| ATOM | 2751 | C | VAL | B | 163 | 104.976 | 93.156 | 63.031 | 1.00 | 11.79 | C |
| ATOM | 2752 | O | VAL | B | 163 | 104.685 | 93.153 | 64.213 | 1.00 | 13.12 | O |
| ATOM | 2753 | N | VAL | B | 164 | 104.070 | 92.979 | 62.089 | 1.00 | 12.77 | N |
| ATOM | 2754 | CA | VAL | B | 164 | 102.680 | 92.596 | 62.395 | 1.00 | 13.92 | C |
| ATOM | 2755 | CB | VAL | B | 164 | 101.724 | 93.799 | 62.265 | 1.00 | 13.81 | C |
| ATOM | 2756 | CG1 | VAL | B | 164 | 102.061 | 94.830 | 63.271 | 1.00 | 16.22 | C |
| ATOM | 2757 | CG2 | VAL | B | 164 | 101.798 | 94.385 | 60.847 | 1.00 | 13.72 | C |
| ATOM | 2758 | C | VAL | B | 164 | 102.285 | 91.531 | 61.406 | 1.00 | 15.62 | C |
| ATOM | 2759 | O | VAL | B | 164 | 102.812 | 91.487 | 60.303 | 1.00 | 15.41 | O |
| ATOM | 2760 | N | THR | B | 165 | 101.348 | 90.676 | 61.790 | 1.00 | 16.76 | N |
| ATOM | 2761 | CA | THR | B | 165 | 100.892 | 89.609 | 60.931 | 1.00 | 18.33 | C |
| ATOM | 2762 | CB | THR | B | 165 | 101.112 | 88.222 | 61.595 | 1.00 | 19.29 | C |
| ATOM | 2763 | OG1 | THR | B | 165 | 100.553 | 88.207 | 62.924 | 1.00 | 19.81 | O |
| ATOM | 2764 | CG2 | THR | B | 165 | 102.594 | 87.936 | 61.795 | 1.00 | 19.76 | C |
| ATOM | 2765 | C | THR | B | 165 | 99.427 | 89.793 | 60.567 | 1.00 | 18.64 | C |
| ATOM | 2766 | O | THR | B | 165 | 98.951 | 89.209 | 59.605 | 1.00 | 19.51 | O |
| ATOM | 2767 | N | SER | B | 166 | 98.705 | 90.588 | 61.340 | 1.00 | 19.54 | N |
| ATOM | 2768 | CA | SER | B | 166 | 97.337 | 90.922 | 61.003 | 1.00 | 20.68 | C |
| ATOM | 2769 | CB | SER | B | 166 | 96.643 | 91.508 | 62.241 | 1.00 | 21.46 | C |
| ATOM | 2770 | OG | SER | B | 166 | 95.316 | 91.852 | 61.893 | 1.00 | 26.95 | O |
| ATOM | 2771 | C | SER | B | 166 | 97.337 | 91.874 | 59.789 | 1.00 | 19.11 | C |
| ATOM | 2772 | O | SER | B | 166 | 98.211 | 92.722 | 59.684 | 1.00 | 19.31 | O |
| ATOM | 2773 | N | LEU | B | 167 | 96.392 | 91.693 | 58.863 | 1.00 | 18.19 | N |
| ATOM | 2774 | CA | LEU | B | 167 | 96.291 | 92.469 | 57.621 | 1.00 | 18.04 | C |
| ATOM | 2775 | CB | LEU | B | 167 | 95.993 | 93.941 | 57.904 | 1.00 | 18.28 | C |
| ATOM | 2776 | CG | LEU | B | 167 | 94.514 | 94.318 | 57.977 | 1.00 | 21.12 | C |
| ATOM | 2777 | CD1 | LEU | B | 167 | 93.747 | 93.304 | 58.783 | 1.00 | 25.72 | C |
| ATOM | 2778 | CD2 | LEU | B | 167 | 94.352 | 95.730 | 58.527 | 1.00 | 20.66 | C |
| ATOM | 2779 | C | LEU | B | 167 | 97.563 | 92.330 | 56.773 | 1.00 | 16.59 | C |
| ATOM | 2780 | O | LEU | B | 167 | 97.950 | 93.237 | 56.041 | 1.00 | 17.37 | O |
| ATOM | 2781 | N | CYS | B | 168 | 98.205 | 91.182 | 56.895 | 1.00 | 16.16 | N |
| ATOM | 2782 | CA | CYS | B | 168 | 99.426 | 90.861 | 56.167 | 1.00 | 16.34 | C |
| ATOM | 2783 | CB | CYS | B | 168 | 100.626 | 90.854 | 57.106 | 1.00 | 15.80 | C |
| ATOM | 2784 | SG | CYS | B | 168 | 102.177 | 90.674 | 56.187 | 1.00 | 13.42 | S |
| ATOM | 2785 | C | CYS | B | 168 | 99.314 | 89.466 | 55.549 | 1.00 | 17.48 | C |
| ATOM | 2786 | O | CYS | B | 168 | 98.808 | 88.565 | 56.176 | 1.00 | 16.93 | O |
| ATOM | 2787 | N | ARG | B | 177 | 99.790 | 89.292 | 54.316 | 1.00 | 18.17 | N |
| ATOM | 2788 | CA | ARG | B | 177 | 99.816 | 87.970 | 53.700 | 1.00 | 18.70 | C |
| ATOM | 2789 | CB | ARG | B | 177 | 100.036 | 88.117 | 52.190 | 1.00 | 19.76 | C |
| ATOM | 2790 | CG | ARG | B | 177 | 98.836 | 88.691 | 51.473 | 1.00 | 22.85 | C |
| ATOM | 2791 | CD | ARG | B | 177 | 98.924 | 88.687 | 49.982 | 1.00 | 28.01 | C |
| ATOM | 2792 | NE | ARG | B | 177 | 100.119 | 89.368 | 49.483 | 1.00 | 28.07 | N |
| ATOM | 2793 | CZ | ARG | B | 177 | 100.184 | 90.080 | 48.339 | 1.00 | 31.02 | C |
| ATOM | 2794 | NH1 | ARG | B | 177 | 99.135 | 90.220 | 47.545 | 1.00 | 33.24 | N |
| ATOM | 2795 | NH2 | ARG | B | 177 | 101.335 | 90.650 | 47.970 | 1.00 | 26.55 | N |
| ATOM | 2796 | C | ARG | B | 177 | 100.945 | 87.158 | 54.313 | 1.00 | 17.98 | C |
| ATOM | 2797 | O | ARG | B | 177 | 101.950 | 87.710 | 54.795 | 1.00 | 15.40 | O |
| ATOM | 2798 | N | ARG | B | 178 | 100.780 | 85.840 | 54.315 | 1.00 | 18.33 | N |
| ATOM | 2799 | CA | ARG | B | 178 | 101.833 | 84.945 | 54.790 | 1.00 | 18.98 | C |
| ATOM | 2800 | CB | ARG | B | 178 | 101.358 | 83.480 | 54.861 | 1.00 | 20.71 | C |
| ATOM | 2801 | CG | ARG | B | 178 | 100.363 | 83.195 | 55.959 | 1.00 | 25.37 | C |
| ATOM | 2802 | CD | ARG | B | 178 | 100.887 | 83.192 | 57.417 | 1.00 | 32.14 | C |
| ATOM | 2803 | NE | ARG | B | 178 | 102.080 | 82.379 | 57.711 | 1.00 | 37.96 | N |
| ATOM | 2804 | CZ | ARG | B | 178 | 102.218 | 81.066 | 57.507 | 1.00 | 40.45 | C |
| ATOM | 2805 | NH1 | ARG | B | 178 | 103.359 | 80.466 | 57.855 | 1.00 | 41.21 | N |
| ATOM | 2806 | NH2 | ARG | B | 178 | 101.253 | 80.347 | 56.942 | 1.00 | 41.93 | N |
| ATOM | 2807 | C | ARG | B | 178 | 103.060 | 85.028 | 53.883 | 1.00 | 17.83 | C |
| ATOM | 2808 | O | ARG | B | 178 | 104.146 | 84.689 | 54.293 | 1.00 | 18.64 | O |
| ATOM | 2809 | N | SER | B | 179 | 102.840 | 85.460 | 52.649 | 1.00 | 16.99 | N |
| ATOM | 2810 | CA | SER | B | 179 | 103.885 | 85.673 | 51.663 | 1.00 | 15.90 | C |
| ATOM | 2811 | CB | SER | B | 179 | 103.277 | 85.462 | 50.286 | 1.00 | 16.47 | C |
| ATOM | 2812 | OG | SER | B | 179 | 102.305 | 86.490 | 50.019 | 1.00 | 17.07 | O |
| ATOM | 2813 | C | SER | B | 179 | 104.553 | 87.069 | 51.771 | 1.00 | 14.90 | C |
| ATOM | 2814 | O | SER | B | 179 | 105.381 | 87.447 | 50.923 | 1.00 | 15.37 | O |
| ATOM | 2815 | N | ASN | B | 180 | 104.202 | 87.854 | 52.792 | 1.00 | 12.77 | N |
| ATOM | 2816 | CA | ASN | B | 180 | 104.970 | 89.054 | 53.092 | 1.00 | 12.00 | C |
| ATOM | 2817 | CB | ASN | B | 180 | 104.118 | 90.342 | 52.949 | 1.00 | 10.21 | C |
| ATOM | 2818 | CG | ASN | B | 180 | 103.893 | 90.730 | 51.511 | 1.00 | 12.69 | C |
| ATOM | 2819 | OD1 | ASN | B | 180 | 102.762 | 90.781 | 51.067 | 1.00 | 11.56 | O |
| ATOM | 2820 | ND2 | ASN | B | 180 | 104.973 | 90.997 | 50.759 | 1.00 | 11.56 | N |
| ATOM | 2821 | C | ASN | B | 180 | 105.523 | 89.050 | 54.495 | 1.00 | 12.01 | C |
| ATOM | 2822 | O | ASN | B | 180 | 105.071 | 88.292 | 55.355 | 1.00 | 11.40 | O |
| ATOM | 2823 | N | VAL | B | 181 | 106.490 | 89.936 | 54.734 | 1.00 | 11.13 | N |
| ATOM | 2824 | CA | VAL | B | 181 | 106.734 | 90.426 | 56.069 | 1.00 | 12.31 | C |
| ATOM | 2825 | CB | VAL | B | 181 | 108.237 | 90.536 | 56.399 | 1.00 | 13.82 | C |
| ATOM | 2826 | CG1 | VAL | B | 181 | 108.852 | 89.190 | 56.371 | 1.00 | 17.32 | C |
| ATOM | 2827 | CG2 | VAL | B | 181 | 108.418 | 91.151 | 57.794 | 1.00 | 15.49 | C |
| ATOM | 2828 | C | VAL | B | 181 | 106.162 | 91.833 | 56.089 | 1.00 | 12.36 | C |
| ATOM | 2829 | O | VAL | B | 181 | 106.447 | 92.606 | 55.185 | 1.00 | 11.21 | O |
| ATOM | 2830 | N | CYS | B | 182 | 105.298 | 92.130 | 57.057 | 1.00 | 11.77 | N |
| ATOM | 2831 | CA | CYS | B | 182 | 104.680 | 93.443 | 57.151 | 1.00 | 12.16 | C |
| ATOM | 2832 | CB | CYS | B | 182 | 103.164 | 93.324 | 57.139 | 1.00 | 12.29 | C |
| ATOM | 2833 | SG | CYS | B | 182 | 102.508 | 92.580 | 55.632 | 1.00 | 13.42 | S |
| ATOM | 2834 | C | CYS | B | 182 | 105.085 | 94.180 | 58.396 | 1.00 | 12.19 | C |
| ATOM | 2835 | O | CYS | B | 182 | 105.402 | 93.566 | 59.425 | 1.00 | 11.73 | O |
| ATOM | 2836 | N | THR | B | 183 | 105.059 | 95.497 | 58.302 | 1.00 | 11.48 | N |
| ATOM | 2837 | CA | THR | B | 183 | 105.275 | 96.355 | 59.452 | 1.00 | 11.92 | C |
| ATOM | 2838 | CB | THR | B | 183 | 106.682 | 96.978 | 59.433 | 1.00 | 11.74 | C |
| ATOM | 2839 | OG1 | THR | B | 183 | 106.816 | 97.894 | 58.323 | 1.00 | 11.45 | O |
| ATOM | 2840 | CG2 | THR | B | 183 | 107.724 | 95.884 | 59.231 | 1.00 | 9.89 | C |
| ATOM | 2841 | C | THR | B | 183 | 104.243 | 97.451 | 59.595 | 1.00 | 12.96 | C |
| ATOM | 2842 | O | THR | B | 183 | 103.633 | 97.893 | 58.620 | 1.00 | 12.13 | O |
| ATOM | 2843 | N | LEU | B | 184 | 104.083 | 97.904 | 60.829 | 1.00 | 14.62 | N |
| ATOM | 2844 | CA | LEU | B | 184 | 103.212 | 99.003 | 61.161 | 1.00 | 15.26 | C |
| ATOM | 2845 | CB | LEU | B | 184 | 101.862 | 98.421 | 61.627 | 1.00 | 16.49 | C |
| ATOM | 2846 | CG | LEU | B | 184 | 100.616 | 99.292 | 61.791 | 1.00 | 17.61 | C |
| ATOM | 2847 | CD1 | LEU | B | 184 | 100.262 | 99.959 | 60.480 | 1.00 | 17.79 | C |
| ATOM | 2848 | CD2 | LEU | B | 184 | 99.464 | 98.443 | 62.314 | 1.00 | 18.22 | C |
| ATOM | 2849 | C | LEU | B | 184 | 103.826 | 99.823 | 62.283 | 1.00 | 16.70 | C |
| ATOM | 2850 | O | LEU | B | 184 | 104.311 | 99.261 | 63.305 | 1.00 | 18.40 | O |
| ATOM | 2851 | N | VAL | B | 185 | 103.812 | 101.132 | 62.122 | 1.00 | 16.79 | N |
| ATOM | 2852 | CA | VAL | B | 185 | 104.077 | 102.055 | 63.202 | 1.00 | 17.62 | C |
| ATOM | 2853 | CB | VAL | B | 185 | 104.632 | 103.375 | 62.684 | 1.00 | 17.31 | C |
| ATOM | 2854 | CG1 | VAL | B | 185 | 104.888 | 104.341 | 63.854 | 1.00 | 17.47 | C |
| ATOM | 2855 | CG2 | VAL | B | 185 | 105.930 | 103.146 | 61.892 | 1.00 | 16.32 | C |
| ATOM | 2856 | C | VAL | B | 185 | 102.768 | 102.318 | 63.971 | 1.00 | 19.04 | C |
| ATOM | 2857 | O | VAL | B | 185 | 101.811 | 102.830 | 63.416 | 1.00 | 18.57 | O |
| ATOM | 2858 | N | ARG | B | 186 | 102.737 | 101.966 | 65.249 | 1.00 | 20.93 | N |
| ATOM | 2859 | CA | ARG | B | 186 | 101.542 | 102.166 | 66.046 | 1.00 | 23.10 | C |
| ATOM | 2860 | CB | ARG | B | 186 | 101.400 | 101.048 | 67.082 | 1.00 | 24.18 | C |
| ATOM | 2861 | CG | ARG | B | 186 | 100.723 | 99.793 | 66.515 | 1.00 | 28.84 | C |
| ATOM | 2862 | CD | ARG | B | 186 | 101.484 | 98.481 | 66.665 | 1.00 | 33.82 | C |
| ATOM | 2863 | NE | ARG | B | 186 | 100.572 | 97.338 | 66.490 | 1.00 | 36.92 | N |
| ATOM | 2864 | CZ | ARG | B | 186 | 100.952 | 96.066 | 66.333 | 1.00 | 40.38 | C |
| ATOM | 2865 | NH1 | ARG | B | 186 | 102.253 | 95.716 | 66.332 | 1.00 | 40.68 | N |
| ATOM | 2866 | NH2 | ARG | B | 186 | 100.015 | 95.130 | 66.185 | 1.00 | 40.12 | N |
| ATOM | 2867 | C | ARG | B | 186 | 101.524 | 103.531 | 66.707 | 1.00 | 22.83 | C |
| ATOM | 2868 | O | ARG | B | 186 | 102.578 | 104.080 | 67.023 | 1.00 | 23.27 | O |
| ATOM | 2869 | N | GLY | B | 186A | 100.318 | 104.077 | 66.848 | 1.00 | 23.49 | N |
| ATOM | 2870 | CA | GLY | B | 186A | 100.048 | 105.296 | 67.605 | 1.00 | 23.44 | C |
| ATOM | 2871 | C | GLY | B | 186A | 100.326 | 106.596 | 66.863 | 1.00 | 23.25 | C |
| ATOM | 2872 | O | GLY | B | 186A | 100.194 | 107.666 | 67.427 | 1.00 | 22.68 | O |
| ATOM | 2873 | N | ARG | B | 187 | 100.747 | 106.496 | 65.605 | 1.00 | 22.29 | N |
| ATOM | 2874 | CA | ARG | B | 187 | 100.821 | 107.634 | 64.746 | 1.00 | 22.27 | C |
| ATOM | 2875 | CB | ARG | B | 187 | 102.078 | 108.453 | 65.036 | 1.00 | 22.98 | C |
| ATOM | 2876 | CG | ARG | B | 187 | 103.370 | 107.699 | 64.856 | 1.00 | 22.53 | C |
| ATOM | 2877 | CD | ARG | B | 187 | 104.548 | 108.386 | 65.520 | 1.00 | 23.28 | C |
| ATOM | 2878 | NE | ARG | B | 187 | 105.799 | 107.719 | 65.133 | 1.00 | 23.85 | N |
| ATOM | 2879 | CZ | ARG | B | 187 | 106.667 | 108.188 | 64.235 | 1.00 | 22.61 | C |
| ATOM | 2880 | NH1 | ARG | B | 187 | 106.454 | 109.318 | 63.586 | 1.00 | 24.07 | N |
| ATOM | 2881 | NH2 | ARG | B | 187 | 107.765 | 107.503 | 63.986 | 1.00 | 22.69 | N |
| ATOM | 2882 | C | ARG | B | 187 | 100.812 | 107.158 | 63.313 | 1.00 | 22.63 | C |
| ATOM | 2883 | O | ARG | B | 187 | 100.668 | 105.961 | 63.059 | 1.00 | 22.93 | O |
| ATOM | 2884 | N | GLN | B | 188 | 100.910 | 108.110 | 62.390 | 1.00 | 21.81 | N |
| ATOM | 2885 | CA | GLN | B | 188 | 100.960 | 107.824 | 60.967 | 1.00 | 21.50 | C |
| ATOM | 2886 | CB | GLN | B | 188 | 99.971 | 108.719 | 60.226 | 1.00 | 22.64 | C |
| ATOM | 2887 | CG | GLN | B | 188 | 98.591 | 108.104 | 60.302 | 1.00 | 24.41 | C |
| ATOM | 2888 | CD | GLN | B | 188 | 97.515 | 109.036 | 59.831 | 1.00 | 27.71 | C |
| ATOM | 2889 | OE1 | GLN | B | 188 | 96.621 | 109.392 | 60.609 | 1.00 | 28.43 | O |
| ATOM | 2890 | NE2 | GLN | B | 188 | 97.592 | 109.447 | 58.556 | 1.00 | 27.75 | N |
| ATOM | 2891 | C | GLN | B | 188 | 102.371 | 107.979 | 60.414 | 1.00 | 20.10 | C |
| ATOM | 2892 | O | GLN | B | 188 | 102.878 | 109.079 | 60.234 | 1.00 | 18.92 | O |
| ATOM | 2893 | N | ALA | B | 188A | 102.988 | 106.828 | 60.166 | 1.00 | 18.01 | N |
| ATOM | 2894 | CA | ALA | B | 188A | 104.360 | 106.742 | 59.700 | 1.00 | 17.43 | C |
| ATOM | 2895 | CB | ALA | B | 188A | 105.317 | 106.724 | 60.898 | 1.00 | 17.40 | C |
| ATOM | 2896 | C | ALA | B | 188A | 104.469 | 105.451 | 58.870 | 1.00 | 15.68 | C |
| ATOM | 2897 | O | ALA | B | 188A | 103.725 | 104.498 | 59.089 | 1.00 | 14.26 | O |
| ATOM | 2898 | N | GLY | B | 189 | 105.350 | 105.450 | 57.887 | 1.00 | 14.86 | N |
| ATOM | 2899 | CA | GLY | B | 189 | 105.580 | 104.275 | 57.089 | 1.00 | 14.39 | C |
| ATOM | 2900 | C | GLY | B | 189 | 106.450 | 104.544 | 55.884 | 1.00 | 14.28 | C |
| ATOM | 2901 | O | GLY | B | 189 | 106.925 | 105.665 | 55.673 | 1.00 | 14.85 | O |
| ATOM | 2902 | N | VAL | B | 190 | 106.644 | 103.519 | 55.070 | 1.00 | 13.66 | N |
| ATOM | 2903 | CA | VAL | B | 190 | 107.335 | 103.688 | 53.805 | 1.00 | 13.23 | C |
| ATOM | 2904 | CB | VAL | B | 190 | 107.906 | 102.338 | 53.255 | 1.00 | 13.79 | C |
| ATOM | 2905 | CG1 | VAL | B | 190 | 108.680 | 101.613 | 54.364 | 1.00 | 13.50 | C |
| ATOM | 2906 | CG2 | VAL | B | 190 | 106.810 | 101.444 | 52.701 | 1.00 | 12.75 | C |
| ATOM | 2907 | C | VAL | B | 190 | 106.416 | 104.381 | 52.785 | 1.00 | 14.21 | C |
| ATOM | 2908 | O | VAL | B | 190 | 105.193 | 104.508 | 52.979 | 1.00 | 13.72 | O |
| ATOM | 2909 | N | CYS | B | 191 | 107.016 | 104.871 | 51.708 | 1.00 | 14.56 | N |
| ATOM | 2910 | CA | CYS | B | 191 | 106.231 | 105.533 | 50.674 | 1.00 | 14.34 | C |
| ATOM | 2911 | CB | CYS | B | 191 | 105.996 | 106.986 | 51.090 | 1.00 | 14.82 | C |
| ATOM | 2912 | SG | CYS | B | 191 | 104.590 | 107.784 | 50.301 | 1.00 | 13.00 | S |
| ATOM | 2913 | C | CYS | B | 191 | 106.967 | 105.427 | 49.330 | 1.00 | 14.14 | C |
| ATOM | 2914 | O | CYS | B | 191 | 108.004 | 104.784 | 49.236 | 1.00 | 12.23 | O |
| ATOM | 2915 | N | PHE | B | 192 | 106.400 | 106.023 | 48.280 | 1.00 | 13.42 | N |
| ATOM | 2916 | CA | PHE | B | 192 | 106.924 | 105.831 | 46.922 | 1.00 | 13.44 | C |
| ATOM | 2917 | CB | PHE | B | 192 | 105.997 | 106.540 | 45.919 | 1.00 | 14.57 | C |
| ATOM | 2918 | CG | PHE | B | 192 | 104.589 | 106.073 | 46.016 | 1.00 | 16.83 | C |
| ATOM | 2919 | CD1 | PHE | B | 192 | 103.646 | 106.813 | 46.702 | 1.00 | 23.12 | C |
| ATOM | 2920 | CE1 | PHE | B | 192 | 102.362 | 106.340 | 46.836 | 1.00 | 23.14 | C |
| ATOM | 2921 | CZ | PHE | B | 192 | 102.013 | 105.129 | 46.291 | 1.00 | 23.11 | C |
| ATOM | 2922 | CE2 | PHE | B | 192 | 102.942 | 104.381 | 45.612 | 1.00 | 23.03 | C |
| ATOM | 2923 | CD2 | PHE | B | 192 | 104.223 | 104.840 | 45.495 | 1.00 | 20.24 | C |
| ATOM | 2924 | C | PHE | B | 192 | 108.369 | 106.311 | 46.864 | 1.00 | 13.21 | C |
| ATOM | 2925 | O | PHE | B | 192 | 108.669 | 107.373 | 47.416 | 1.00 | 14.33 | O |
| ATOM | 2926 | N | GLY | B | 193 | 109.233 | 105.536 | 46.202 | 1.00 | 12.67 | N |
| ATOM | 2927 | CA | GLY | B | 193 | 110.675 | 105.725 | 46.228 | 1.00 | 12.78 | C |
| ATOM | 2928 | C | GLY | B | 193 | 111.399 | 104.887 | 47.256 | 1.00 | 12.58 | C |
| ATOM | 2929 | O | GLY | B | 193 | 112.639 | 104.709 | 47.169 | 1.00 | 11.83 | O |
| ATOM | 2930 | N | ASP | B | 194 | 110.659 | 104.330 | 48.222 | 1.00 | 12.13 | N |
| ATOM | 2931 | CA | ASP | B | 194 | 111.263 | 103.464 | 49.221 | 1.00 | 11.67 | C |
| ATOM | 2932 | CB | ASP | B | 194 | 110.538 | 103.536 | 50.563 | 1.00 | 12.39 | C |
| ATOM | 2933 | CG | ASP | B | 194 | 110.709 | 104.866 | 51.236 | 1.00 | 12.47 | C |
| ATOM | 2934 | OD1 | ASP | B | 194 | 111.826 | 105.458 | 51.173 | 1.00 | 9.07 | O |
| ATOM | 2935 | OD2 | ASP | B | 194 | 109.782 | 105.396 | 51.841 | 1.00 | 11.49 | O |
| ATOM | 2936 | C | ASP | B | 194 | 111.291 | 102.039 | 48.774 | 1.00 | 11.52 | C |
| ATOM | 2937 | O | ASP | B | 194 | 111.981 | 101.258 | 49.400 | 1.00 | 10.69 | O |
| ATOM | 2938 | N | SER | B | 195 | 110.508 | 101.669 | 47.752 | 1.00 | 11.50 | N |
| ATOM | 2939 | CA | SER | B | 195 | 110.506 | 100.276 | 47.257 | 1.00 | 11.88 | C |
| ATOM | 2940 | CB | SER | B | 195 | 109.609 | 100.145 | 46.036 | 1.00 | 12.80 | C |
| ATOM | 2941 | OG | SER | B | 195 | 108.239 | 100.280 | 46.419 | 1.00 | 12.81 | O |
| ATOM | 2942 | C | SER | B | 195 | 111.937 | 99.826 | 46.952 | 1.00 | 10.85 | C |
| ATOM | 2943 | O | SER | B | 195 | 112.780 | 100.617 | 46.481 | 1.00 | 11.79 | O |
| ATOM | 2944 | N | GLY | B | 196 | 112.241 | 98.581 | 47.304 | 1.00 | 10.22 | N |
| ATOM | 2945 | CA | GLY | B | 196 | 113.537 | 98.014 | 47.040 | 1.00 | 8.96 | C |
| ATOM | 2946 | C | GLY | B | 196 | 114.475 | 98.152 | 48.224 | 1.00 | 8.99 | C |
| ATOM | 2947 | O | GLY | B | 196 | 115.482 | 97.437 | 48.294 | 1.00 | 7.95 | O |
| ATOM | 2948 | N | SER | B | 197 | 114.125 | 99.019 | 49.165 | 1.00 | 9.18 | N |
| ATOM | 2949 | CA | SER | B | 197 | 115.004 | 99.364 | 50.291 | 1.00 | 9.90 | C |
| ATOM | 2950 | CB | SER | B | 197 | 114.579 | 100.667 | 50.945 | 1.00 | 9.25 | C |
| ATOM | 2951 | OG | SER | B | 197 | 114.511 | 101.770 | 50.038 | 1.00 | 10.16 | O |
| ATOM | 2952 | C | SER | B | 197 | 114.937 | 98.246 | 51.348 | 1.00 | 9.85 | C |
| ATOM | 2953 | O | SER | B | 197 | 113.907 | 97.589 | 51.463 | 1.00 | 9.18 | O |
| ATOM | 2954 | N | PRO | B | 198 | 116.010 | 98.061 | 52.130 | 1.00 | 10.38 | N |
| ATOM | 2955 | CA | PRO | B | 198 | 116.018 | 97.026 | 53.172 | 1.00 | 11.22 | C |
| ATOM | 2956 | CB | PRO | B | 198 | 117.502 | 96.962 | 53.565 | 1.00 | 10.57 | C |
| ATOM | 2957 | CG | PRO | B | 198 | 117.941 | 98.416 | 53.438 | 1.00 | 12.00 | C |
| ATOM | 2958 | CD | PRO | B | 198 | 117.281 | 98.831 | 52.128 | 1.00 | 11.07 | C |
| ATOM | 2959 | C | PRO | B | 198 | 115.162 | 97.355 | 54.384 | 1.00 | 10.77 | C |
| ATOM | 2960 | O | PRO | B | 198 | 114.983 | 98.551 | 54.736 | 1.00 | 12.44 | O |
| ATOM | 2961 | N | LEU | B | 199 | 114.642 | 96.284 | 55.005 | 1.00 | 11.75 | N |
| ATOM | 2962 | CA | LEU | B | 199 | 114.088 | 96.263 | 56.351 | 1.00 | 10.97 | C |
| ATOM | 2963 | CB | LEU | B | 199 | 112.717 | 95.555 | 56.384 | 1.00 | 11.30 | C |
| ATOM | 2964 | CG | LEU | B | 199 | 112.055 | 95.456 | 57.772 | 1.00 | 11.69 | C |
| ATOM | 2965 | CD1 | LEU | B | 199 | 111.636 | 96.837 | 58.253 | 1.00 | 12.48 | C |
| ATOM | 2966 | CD2 | LEU | B | 199 | 110.880 | 94.495 | 57.688 | 1.00 | 14.31 | C |
| ATOM | 2967 | C | LEU | B | 199 | 115.059 | 95.495 | 57.218 | 1.00 | 11.39 | C |
| ATOM | 2968 | O | LEU | B | 199 | 115.303 | 94.331 | 56.963 | 1.00 | 10.76 | O |
| ATOM | 2969 | N | VAL | B | 200 | 115.650 | 96.174 | 58.203 | 1.00 | 12.93 | N |
| ATOM | 2970 | CA | VAL | B | 200 | 116.679 | 95.586 | 59.076 | 1.00 | 12.91 | C |
| ATOM | 2971 | CB | VAL | B | 200 | 117.826 | 96.576 | 59.336 | 1.00 | 13.68 | C |
| ATOM | 2972 | CG1 | VAL | B | 200 | 118.996 | 95.866 | 60.106 | 1.00 | 14.91 | C |
| ATOM | 2973 | CG2 | VAL | B | 200 | 118.314 | 97.192 | 58.035 | 1.00 | 12.22 | C |
| ATOM | 2974 | C | VAL | B | 200 | 116.048 | 95.189 | 60.421 | 1.00 | 12.96 | C |
| ATOM | 2975 | O | VAL | B | 200 | 115.355 | 95.975 | 61.063 | 1.00 | 12.68 | O |
| ATOM | 2976 | N | CYS | B | 201 | 116.208 | 93.923 | 60.793 | 1.00 | 12.83 | N |
| ATOM | 2977 | CA | CYS | B | 201 | 115.662 | 93.413 | 62.046 | 1.00 | 14.09 | C |
| ATOM | 2978 | CB | CYS | B | 201 | 114.450 | 92.525 | 61.805 | 1.00 | 13.90 | C |
| ATOM | 2979 | SG | CYS | B | 201 | 113.154 | 93.232 | 60.753 | 1.00 | 13.93 | S |
| ATOM | 2980 | C | CYS | B | 201 | 116.787 | 92.602 | 62.709 | 1.00 | 14.77 | C |
| ATOM | 2981 | O | CYS | B | 201 | 117.348 | 91.715 | 62.084 | 1.00 | 15.70 | O |
| ATOM | 2982 | N | ASN | B | 204 | 117.161 | 92.949 | 63.934 | 1.00 | 16.27 | N |
| ATOM | 2983 | CA | ASN | B | 204 | 118.215 | 92.207 | 64.622 | 1.00 | 16.47 | C |
| ATOM | 2984 | CB | ASN | B | 204 | 117.766 | 90.754 | 64.879 | 1.00 | 17.25 | C |
| ATOM | 2985 | CG | ASN | B | 204 | 116.335 | 90.623 | 65.423 | 1.00 | 20.04 | C |
| ATOM | 2986 | OD1 | ASN | B | 204 | 115.585 | 89.735 | 64.998 | 1.00 | 24.12 | O |
| ATOM | 2987 | ND2 | ASN | B | 204 | 115.968 | 91.481 | 66.364 | 1.00 | 20.59 | N |
| ATOM | 2988 | C | ASN | B | 204 | 119.517 | 92.163 | 63.806 | 1.00 | 16.39 | C |
| ATOM | 2989 | O | ASN | B | 204 | 120.176 | 91.134 | 63.785 | 1.00 | 17.38 | O |
| ATOM | 2990 | N | GLY | B | 205 | 119.827 | 93.240 | 63.086 | 1.00 | 16.33 | N |
| ATOM | 2991 | CA | GLY | B | 205 | 121.022 | 93.373 | 62.281 | 1.00 | 17.23 | C |
| ATOM | 2992 | C | GLY | B | 205 | 121.021 | 92.759 | 60.887 | 1.00 | 16.96 | C |
| ATOM | 2993 | O | GLY | B | 205 | 121.999 | 92.897 | 60.156 | 1.00 | 17.86 | O |
| ATOM | 2994 | N | LEU | B | 208 | 119.941 | 92.069 | 60.536 | 1.00 | 16.47 | N |
| ATOM | 2995 | CA | LEU | B | 208 | 119.842 | 91.265 | 59.321 | 1.00 | 16.38 | C |
| ATOM | 2996 | CB | LEU | B | 208 | 119.466 | 89.830 | 59.711 | 1.00 | 17.56 | C |
| ATOM | 2997 | CG | LEU | B | 208 | 120.587 | 88.805 | 59.978 | 1.00 | 20.39 | C |
| ATOM | 2998 | CD1 | LEU | B | 208 | 121.865 | 89.419 | 60.457 | 1.00 | 20.06 | C |
| ATOM | 2999 | CD2 | LEU | B | 208 | 120.104 | 87.729 | 60.881 | 1.00 | 21.91 | C |
| ATOM | 3000 | C | LEU | B | 208 | 118.751 | 91.820 | 58.385 | 1.00 | 15.18 | C |
| ATOM | 3001 | O | LEU | B | 208 | 117.798 | 92.406 | 58.847 | 1.00 | 14.63 | O |
| ATOM | 3002 | N | ILE | B | 209 | 118.874 | 91.584 | 57.082 | 1.00 | 13.37 | N |
| ATOM | 3003 | CA | ILE | B | 209 | 117.900 | 92.139 | 56.124 | 1.00 | 13.14 | C |
| ATOM | 3004 | CB | ILE | B | 209 | 118.534 | 92.499 | 54.759 | 1.00 | 12.07 | C |
| ATOM | 3005 | CG1 | ILE | B | 209 | 119.878 | 93.252 | 54.881 | 1.00 | 13.96 | C |
| ATOM | 3006 | CD1 | ILE | B | 209 | 119.928 | 94.485 | 55.683 | 1.00 | 13.87 | C |
| ATOM | 3007 | CG2 | ILE | B | 209 | 117.493 | 93.301 | 53.885 | 1.00 | 11.52 | C |
| ATOM | 3008 | C | ILE | B | 209 | 116.773 | 91.132 | 55.935 | 1.00 | 11.83 | C |
| ATOM | 3009 | O | ILE | B | 209 | 116.919 | 90.145 | 55.239 | 1.00 | 11.78 | O |
| ATOM | 3010 | N | HIS | B | 210 | 115.675 | 91.343 | 56.638 | 1.00 | 12.12 | N |
| ATOM | 3011 | CA | HIS | B | 210 | 114.533 | 90.432 | 56.589 | 1.00 | 12.19 | C |
| ATOM | 3012 | CB | HIS | B | 210 | 113.847 | 90.345 | 57.982 | 1.00 | 13.52 | C |
| ATOM | 3013 | CG | HIS | B | 210 | 114.561 | 89.439 | 58.938 | 1.00 | 15.23 | C |
| ATOM | 3014 | ND1 | HIS | B | 210 | 114.266 | 88.091 | 59.056 | 1.00 | 15.46 | N |
| ATOM | 3015 | CE1 | HIS | B | 210 | 115.042 | 87.562 | 59.996 | 1.00 | 13.95 | C |
| ATOM | 3016 | NE2 | HIS | B | 210 | 115.826 | 88.517 | 60.481 | 1.00 | 13.71 | N |
| ATOM | 3017 | CD2 | HIS | B | 210 | 115.556 | 89.690 | 59.822 | 1.00 | 15.23 | C |
| ATOM | 3018 | C | HIS | B | 210 | 113.472 | 90.837 | 55.552 | 1.00 | 11.93 | C |
| ATOM | 3019 | O | HIS | B | 210 | 112.611 | 90.009 | 55.165 | 1.00 | 10.66 | O |
| ATOM | 3020 | N | GLY | B | 211 | 113.477 | 92.105 | 55.152 | 1.00 | 11.39 | N |
| ATOM | 3021 | CA | GLY | B | 211 | 112.577 | 92.540 | 54.093 | 1.00 | 10.37 | C |
| ATOM | 3022 | C | GLY | B | 211 | 113.198 | 93.445 | 53.041 | 1.00 | 10.52 | C |
| ATOM | 3023 | O | GLY | B | 211 | 114.200 | 94.075 | 53.285 | 1.00 | 11.08 | O |
| ATOM | 3024 | N | ILE | B | 212 | 112.536 | 93.501 | 51.886 | 1.00 | 10.54 | N |
| ATOM | 3025 | CA | ILE | B | 212 | 112.750 | 94.461 | 50.816 | 1.00 | 9.80 | C |
| ATOM | 3026 | CB | ILE | B | 212 | 113.109 | 93.712 | 49.539 | 1.00 | 10.70 | C |
| ATOM | 3027 | CG1 | ILE | B | 212 | 114.402 | 92.913 | 49.734 | 1.00 | 10.16 | C |
| ATOM | 3028 | CD1 | ILE | B | 212 | 114.621 | 91.872 | 48.631 | 1.00 | 13.55 | C |
| ATOM | 3029 | CG2 | ILE | B | 212 | 113.173 | 94.678 | 48.317 | 1.00 | 9.93 | C |
| ATOM | 3030 | C | ILE | B | 212 | 111.402 | 95.178 | 50.615 | 1.00 | 8.95 | C |
| ATOM | 3031 | O | ILE | B | 212 | 110.380 | 94.549 | 50.372 | 1.00 | 8.55 | O |
| ATOM | 3032 | N | ALA | B | 213 | 111.396 | 96.493 | 50.701 | 1.00 | 9.21 | N |
| ATOM | 3033 | CA | ALA | B | 213 | 110.137 | 97.249 | 50.623 | 1.00 | 9.34 | C |
| ATOM | 3034 | CB | ALA | B | 213 | 110.373 | 98.732 | 50.802 | 1.00 | 9.06 | C |
| ATOM | 3035 | C | ALA | B | 213 | 109.419 | 97.005 | 49.303 | 1.00 | 10.27 | C |
| ATOM | 3036 | O | ALA | B | 213 | 109.990 | 97.167 | 48.225 | 1.00 | 9.41 | O |
| ATOM | 3037 | N | SER | B | 214 | 108.145 | 96.655 | 49.402 | 1.00 | 10.01 | N |
| ATOM | 3038 | CA | SER | B | 214 | 107.419 | 96.232 | 48.225 | 1.00 | 10.88 | C |
| ATOM | 3039 | CB | SER | B | 214 | 107.000 | 94.758 | 48.321 | 1.00 | 10.33 | C |
| ATOM | 3040 | OG | SER | B | 214 | 106.253 | 94.364 | 47.164 | 1.00 | 11.41 | O |
| ATOM | 3041 | C | SER | B | 214 | 106.236 | 97.152 | 47.916 | 1.00 | 10.08 | C |
| ATOM | 3042 | O | SER | B | 214 | 106.175 | 97.678 | 46.846 | 1.00 | 12.62 | O |
| ATOM | 3043 | N | PHE | B | 215 | 105.293 | 97.318 | 48.835 | 1.00 | 11.53 | N |
| ATOM | 3044 | CA | PHE | B | 215 | 104.144 | 98.190 | 48.587 | 1.00 | 10.51 | C |
| ATOM | 3045 | CB | PHE | B | 215 | 103.092 | 97.541 | 47.647 | 1.00 | 10.42 | C |
| ATOM | 3046 | CG | PHE | B | 215 | 102.419 | 96.315 | 48.217 | 1.00 | 9.00 | C |
| ATOM | 3047 | CD1 | PHE | B | 215 | 102.975 | 95.048 | 48.018 | 1.00 | 7.71 | C |
| ATOM | 3048 | CE1 | PHE | B | 215 | 102.364 | 93.912 | 48.499 | 1.00 | 10.82 | C |
| ATOM | 3049 | CZ | PHE | B | 215 | 101.173 | 94.009 | 49.194 | 1.00 | 12.17 | C |
| ATOM | 3050 | CE2 | PHE | B | 215 | 100.596 | 95.282 | 49.375 | 1.00 | 11.75 | C |
| ATOM | 3051 | CD2 | PHE | B | 215 | 101.219 | 96.403 | 48.888 | 1.00 | 9.90 | C |
| ATOM | 3052 | C | PHE | B | 215 | 103.472 | 98.646 | 49.844 | 1.00 | 11.06 | C |
| ATOM | 3053 | O | PHE | B | 215 | 103.560 | 98.013 | 50.908 | 1.00 | 8.82 | O |
| ATOM | 3054 | N | VAL | B | 216 | 102.788 | 99.771 | 49.682 | 1.00 | 10.39 | N |
| ATOM | 3055 | CA | VAL | B | 216 | 101.812 | 100.257 | 50.639 | 1.00 | 11.18 | C |
| ATOM | 3056 | CB | VAL | B | 216 | 101.994 | 101.749 | 50.933 | 1.00 | 11.44 | C |
| ATOM | 3057 | CG1 | VAL | B | 216 | 103.442 | 102.033 | 51.369 | 1.00 | 12.90 | C |
| ATOM | 3058 | CG2 | VAL | B | 216 | 101.616 | 102.661 | 49.728 | 1.00 | 11.28 | C |
| ATOM | 3059 | C | VAL | B | 216 | 100.413 | 99.978 | 50.084 | 1.00 | 11.76 | C |
| ATOM | 3060 | O | VAL | B | 216 | 100.222 | 99.853 | 48.847 | 1.00 | 10.76 | O |
| ATOM | 3061 | N | ARG | B | 217 | 99.456 | 99.880 | 50.993 | 1.00 | 12.53 | N |
| ATOM | 3062 | CA | ARG | B | 217 | 98.043 | 99.650 | 50.652 | 1.00 | 14.00 | C |
| ATOM | 3063 | CB | ARG | B | 217 | 97.408 | 98.591 | 51.566 | 1.00 | 14.54 | C |
| ATOM | 3064 | CG | ARG | B | 217 | 97.923 | 97.218 | 51.346 | 1.00 | 17.05 | C |
| ATOM | 3065 | CD | ARG | B | 217 | 97.142 | 96.167 | 52.079 | 1.00 | 19.10 | C |
| ATOM | 3066 | NE | ARG | B | 217 | 97.761 | 94.841 | 52.043 | 1.00 | 22.39 | N |
| ATOM | 3067 | CZ | ARG | B | 217 | 97.600 | 93.927 | 51.062 | 1.00 | 24.79 | C |
| ATOM | 3068 | NH1 | ARG | B | 217 | 96.865 | 94.190 | 49.989 | 1.00 | 23.22 | N |
| ATOM | 3069 | NH2 | ARG | B | 217 | 98.204 | 92.738 | 51.155 | 1.00 | 23.18 | N |
| ATOM | 3070 | C | ARG | B | 217 | 97.288 | 100.965 | 50.792 | 1.00 | 13.34 | C |
| ATOM | 3071 | O | ARG | B | 217 | 97.243 | 101.551 | 51.870 | 1.00 | 12.81 | O |
| ATOM | 3072 | N | GLY | B | 218 | 96.732 | 101.432 | 49.678 | 1.00 | 14.43 | N |
| ATOM | 3073 | CA | GLY | B | 218 | 96.086 | 102.718 | 49.591 | 1.00 | 15.27 | C |
| ATOM | 3074 | C | GLY | B | 218 | 97.162 | 103.777 | 49.576 | 1.00 | 15.92 | C |
| ATOM | 3075 | O | GLY | B | 218 | 98.099 | 103.682 | 48.766 | 1.00 | 17.76 | O |
| ATOM | 3076 | N | GLY | B | 219 | 97.056 | 104.756 | 50.464 | 1.00 | 15.69 | N |
| ATOM | 3077 | CA | GLY | B | 219 | 98.082 | 105.771 | 50.575 | 1.00 | 16.02 | C |
| ATOM | 3078 | C | GLY | B | 219 | 99.174 | 105.328 | 51.528 | 1.00 | 15.72 | C |
| ATOM | 3079 | O | GLY | B | 219 | 99.019 | 104.305 | 52.239 | 1.00 | 15.11 | O |
| ATOM | 3080 | N | CYS | B | 220 | 100.246 | 106.113 | 51.572 | 1.00 | 13.71 | N |
| ATOM | 3081 | CA | CYS | B | 220 | 101.310 | 105.873 | 52.513 | 1.00 | 13.78 | C |
| ATOM | 3082 | CB | CYS | B | 220 | 102.542 | 106.650 | 52.121 | 1.00 | 13.82 | C |
| ATOM | 3083 | SG | CYS | B | 220 | 103.049 | 106.469 | 50.430 | 1.00 | 13.10 | S |
| ATOM | 3084 | C | CYS | B | 220 | 100.841 | 106.312 | 53.892 | 1.00 | 13.64 | C |
| ATOM | 3085 | O | CYS | B | 220 | 100.074 | 107.280 | 54.017 | 1.00 | 13.61 | O |
| ATOM | 3086 | N | ALA | B | 221 | 101.283 | 105.595 | 54.921 | 1.00 | 11.46 | N |
| ATOM | 3087 | CA | ALA | B | 221 | 101.088 | 106.018 | 56.276 | 1.00 | 11.85 | C |
| ATOM | 3088 | CB | ALA | B | 221 | 101.860 | 107.288 | 56.556 | 1.00 | 12.03 | C |
| ATOM | 3089 | C | ALA | B | 221 | 99.598 | 106.140 | 56.624 | 1.00 | 12.45 | C |
| ATOM | 3090 | O | ALA | B | 221 | 99.148 | 107.111 | 57.255 | 1.00 | 12.66 | O |
| ATOM | 3091 | N | SER | B | 222 | 98.842 | 105.096 | 56.266 | 1.00 | 11.92 | N |
| ATOM | 3092 | CA | SER | B | 222 | 97.410 | 105.076 | 56.533 | 1.00 | 12.48 | C |
| ATOM | 3093 | CB | SER | B | 222 | 96.761 | 103.946 | 55.734 | 1.00 | 12.17 | C |
| ATOM | 3094 | OG | SER | B | 222 | 97.226 | 102.663 | 56.119 | 1.00 | 12.18 | O |
| ATOM | 3095 | C | SER | B | 222 | 97.056 | 104.926 | 58.004 | 1.00 | 12.59 | C |
| ATOM | 3096 | O | SER | B | 222 | 95.956 | 105.267 | 58.448 | 1.00 | 13.19 | O |
| ATOM | 3097 | N | GLY | B | 222A | 97.980 | 104.375 | 58.780 | 1.00 | 13.96 | N |
| ATOM | 3098 | CA | GLY | B | 222A | 97.747 | 104.097 | 60.172 | 1.00 | 14.26 | C |
| ATOM | 3099 | C | GLY | B | 222A | 96.978 | 102.808 | 60.354 | 1.00 | 15.35 | C |
| ATOM | 3100 | O | GLY | B | 222A | 96.846 | 102.346 | 61.473 | 1.00 | 19.29 | O |
| ATOM | 3101 | N | LEU | B | 223 | 96.478 | 102.205 | 59.282 | 1.00 | 14.45 | N |
| ATOM | 3102 | CA | LEU | B | 223 | 95.672 | 100.990 | 59.392 | 1.00 | 13.04 | C |
| ATOM | 3103 | CB | LEU | B | 223 | 94.259 | 101.161 | 58.823 | 1.00 | 13.97 | C |
| ATOM | 3104 | CG | LEU | B | 223 | 93.355 | 99.923 | 58.772 | 1.00 | 16.67 | C |
| ATOM | 3105 | CD1 | LEU | B | 223 | 92.896 | 99.524 | 60.133 | 1.00 | 21.51 | C |
| ATOM | 3106 | CD2 | LEU | B | 223 | 92.173 | 100.148 | 57.812 | 1.00 | 20.02 | C |
| ATOM | 3107 | C | LEU | B | 223 | 96.368 | 99.842 | 58.687 | 1.00 | 12.95 | C |
| ATOM | 3108 | O | LEU | B | 223 | 96.548 | 98.757 | 59.279 | 1.00 | 12.69 | O |
| ATOM | 3109 | N | TYR | B | 224 | 96.744 | 100.047 | 57.435 | 1.00 | 11.21 | N |
| ATOM | 3110 | CA | TYR | B | 224 | 97.246 | 98.939 | 56.627 | 1.00 | 10.93 | C |
| ATOM | 3111 | CB | TYR | B | 224 | 97.002 | 99.183 | 55.151 | 1.00 | 11.06 | C |
| ATOM | 3112 | CG | TYR | B | 224 | 95.573 | 99.228 | 54.726 | 1.00 | 10.47 | C |
| ATOM | 3113 | CD1 | TYR | B | 224 | 94.828 | 98.071 | 54.631 | 1.00 | 10.04 | C |
| ATOM | 3114 | CE1 | TYR | B | 224 | 93.497 | 98.105 | 54.204 | 1.00 | 11.77 | C |
| ATOM | 3115 | CZ | TYR | B | 224 | 92.926 | 99.321 | 53.894 | 1.00 | 8.74 | C |
| ATOM | 3116 | OH | TYR | B | 224 | 91.633 | 99.372 | 53.438 | 1.00 | 11.55 | O |
| ATOM | 3117 | CE2 | TYR | B | 224 | 93.673 | 100.506 | 53.962 | 1.00 | 10.42 | C |
| ATOM | 3118 | CD2 | TYR | B | 224 | 94.970 | 100.454 | 54.406 | 1.00 | 10.77 | C |
| ATOM | 3119 | C | TYR | B | 224 | 98.768 | 98.816 | 56.817 | 1.00 | 10.48 | C |
| ATOM | 3120 | O | TYR | B | 224 | 99.477 | 99.787 | 56.601 | 1.00 | 10.92 | O |
| ATOM | 3121 | N | PRO | B | 225 | 99.253 | 97.650 | 57.200 | 1.00 | 10.97 | N |
| ATOM | 3122 | CA | PRO | B | 225 | 100.699 | 97.439 | 57.309 | 1.00 | 10.17 | C |
| ATOM | 3123 | CB | PRO | B | 225 | 100.813 | 95.978 | 57.783 | 1.00 | 11.45 | C |
| ATOM | 3124 | CG | PRO | B | 225 | 99.497 | 95.627 | 58.336 | 1.00 | 11.39 | C |
| ATOM | 3125 | CD | PRO | B | 225 | 98.496 | 96.429 | 57.534 | 1.00 | 10.23 | C |
| ATOM | 3126 | C | PRO | B | 225 | 101.364 | 97.596 | 55.950 | 1.00 | 10.26 | C |
| ATOM | 3127 | O | PRO | B | 225 | 100.797 | 97.264 | 54.894 | 1.00 | 10.79 | O |
| ATOM | 3128 | N | ASP | B | 226 | 102.561 | 98.120 | 55.970 | 1.00 | 10.01 | N |
| ATOM | 3129 | CA | ASP | B | 226 | 103.413 | 98.134 | 54.804 | 1.00 | 9.99 | C |
| ATOM | 3130 | CB | ASP | B | 226 | 104.609 | 99.021 | 55.087 | 1.00 | 9.81 | C |
| ATOM | 3131 | CG | ASP | B | 226 | 104.257 | 100.474 | 55.226 | 1.00 | 12.37 | C |
| ATOM | 3132 | OD1 | ASP | B | 226 | 105.119 | 101.209 | 55.784 | 1.00 | 10.92 | O |
| ATOM | 3133 | OD2 | ASP | B | 226 | 103.193 | 100.987 | 54.789 | 1.00 | 9.73 | O |
| ATOM | 3134 | C | ASP | B | 226 | 103.889 | 96.715 | 54.487 | 1.00 | 9.76 | C |
| ATOM | 3135 | O | ASP | B | 226 | 104.171 | 95.949 | 55.399 | 1.00 | 11.28 | O |
| ATOM | 3136 | N | ALA | B | 227 | 104.028 | 96.379 | 53.215 | 1.00 | 9.00 | N |
| ATOM | 3137 | CA | ALA | B | 227 | 104.461 | 95.045 | 52.814 | 1.00 | 9.93 | C |
| ATOM | 3138 | CB | ALA | B | 227 | 103.462 | 94.416 | 51.816 | 1.00 | 10.20 | C |
| ATOM | 3139 | C | ALA | B | 227 | 105.849 | 94.990 | 52.243 | 1.00 | 10.50 | C |
| ATOM | 3140 | O | ALA | B | 227 | 106.202 | 95.807 | 51.402 | 1.00 | 9.63 | O |
| ATOM | 3141 | N | PHE | B | 228 | 106.587 | 93.958 | 52.663 | 1.00 | 10.08 | N |
| ATOM | 3142 | CA | PHE | B | 228 | 107.992 | 93.742 | 52.292 | 1.00 | 10.94 | C |
| ATOM | 3143 | CB | PHE | B | 228 | 108.955 | 93.919 | 53.490 | 1.00 | 10.55 | C |
| ATOM | 3144 | CG | PHE | B | 228 | 109.022 | 95.315 | 54.051 | 1.00 | 10.66 | C |
| ATOM | 3145 | CD1 | PHE | B | 228 | 110.118 | 96.161 | 53.782 | 1.00 | 9.87 | C |
| ATOM | 3146 | CE1 | PHE | B | 228 | 110.163 | 97.424 | 54.329 | 1.00 | 11.43 | C |
| ATOM | 3147 | CZ | PHE | B | 228 | 109.117 | 97.882 | 55.158 | 1.00 | 9.68 | C |
| ATOM | 3148 | CE2 | PHE | B | 228 | 108.074 | 97.033 | 55.460 | 1.00 | 10.32 | C |
| ATOM | 3149 | CD2 | PHE | B | 228 | 108.031 | 95.769 | 54.918 | 1.00 | 10.77 | C |
| ATOM | 3150 | C | PHE | B | 228 | 108.115 | 92.319 | 51.732 | 1.00 | 10.94 | C |
| ATOM | 3151 | O | PHE | B | 228 | 107.415 | 91.397 | 52.186 | 1.00 | 10.10 | O |
| ATOM | 3152 | N | ALA | B | 229 | 108.979 | 92.141 | 50.721 | 1.00 | 11.79 | N |
| ATOM | 3153 | CA | ALA | B | 229 | 109.284 | 90.800 | 50.289 | 1.00 | 11.25 | C |
| ATOM | 3154 | CB | ALA | B | 229 | 110.146 | 90.781 | 49.028 | 1.00 | 11.74 | C |
| ATOM | 3155 | C | ALA | B | 229 | 110.007 | 90.124 | 51.435 | 1.00 | 11.30 | C |
| ATOM | 3156 | O | ALA | B | 229 | 110.882 | 90.731 | 52.038 | 1.00 | 9.64 | O |
| ATOM | 3157 | N | PRO | B | 230 | 109.664 | 88.863 | 51.723 | 1.00 | 11.41 | N |
| ATOM | 3158 | CA | PRO | B | 230 | 110.252 | 88.150 | 52.853 | 1.00 | 11.41 | C |
| ATOM | 3159 | CB | PRO | B | 230 | 109.226 | 87.042 | 53.125 | 1.00 | 11.41 | C |
| ATOM | 3160 | CG | PRO | B | 230 | 108.567 | 86.774 | 51.806 | 1.00 | 11.95 | C |
| ATOM | 3161 | CD | PRO | B | 230 | 108.634 | 88.059 | 51.046 | 1.00 | 12.27 | C |
| ATOM | 3162 | C | PRO | B | 230 | 111.574 | 87.553 | 52.482 | 1.00 | 11.33 | C |
| ATOM | 3163 | O | PRO | B | 230 | 111.612 | 86.457 | 51.940 | 1.00 | 11.03 | O |
| ATOM | 3164 | N | VAL | B | 231 | 112.658 | 88.249 | 52.777 | 1.00 | 11.75 | N |
| ATOM | 3165 | CA | VAL | B | 231 | 113.980 | 87.821 | 52.282 | 1.00 | 11.67 | C |
| ATOM | 3166 | CB | VAL | B | 231 | 115.078 | 88.809 | 52.789 | 1.00 | 10.37 | C |
| ATOM | 3167 | CG1 | VAL | B | 231 | 114.873 | 90.169 | 52.162 | 1.00 | 10.12 | C |
| ATOM | 3168 | CG2 | VAL | B | 231 | 116.526 | 88.300 | 52.479 | 1.00 | 11.79 | C |
| ATOM | 3169 | C | VAL | B | 231 | 114.347 | 86.385 | 52.717 | 1.00 | 12.95 | C |
| ATOM | 3170 | O | VAL | B | 231 | 114.977 | 85.637 | 51.970 | 1.00 | 13.22 | O |
| ATOM | 3171 | N | ALA | B | 232 | 113.977 | 86.021 | 53.944 | 1.00 | 14.76 | N |
| ATOM | 3172 | CA | ALA | B | 232 | 114.270 | 84.663 | 54.483 | 1.00 | 15.39 | C |
| ATOM | 3173 | CB | ALA | B | 232 | 113.747 | 84.533 | 55.905 | 1.00 | 15.66 | C |
| ATOM | 3174 | C | ALA | B | 232 | 113.687 | 83.537 | 53.637 | 1.00 | 16.07 | C |
| ATOM | 3175 | O | ALA | B | 232 | 114.240 | 82.440 | 53.619 | 1.00 | 17.10 | O |
| ATOM | 3176 | N | GLN | B | 233 | 112.580 | 83.770 | 52.942 | 1.00 | 15.32 | N |
| ATOM | 3177 | CA | GLN | B | 233 | 112.030 | 82.729 | 52.062 | 1.00 | 17.02 | C |
| ATOM | 3178 | CB | GLN | B | 233 | 110.592 | 83.043 | 51.682 | 1.00 | 17.15 | C |
| ATOM | 3179 | CG | GLN | B | 233 | 109.676 | 82.954 | 52.885 | 1.00 | 23.33 | C |
| ATOM | 3180 | CD | GLN | B | 233 | 109.621 | 81.563 | 53.529 | 1.00 | 28.92 | C |
| ATOM | 3181 | OE1 | GLN | B | 233 | 109.702 | 81.430 | 54.764 | 1.00 | 34.54 | O |
| ATOM | 3182 | NE2 | GLN | B | 233 | 109.450 | 80.538 | 52.706 | 1.00 | 34.24 | N |
| ATOM | 3183 | C | GLN | B | 233 | 112.858 | 82.465 | 50.797 | 1.00 | 16.16 | C |
| ATOM | 3184 | O | GLN | B | 233 | 112.635 | 81.466 | 50.122 | 1.00 | 17.18 | O |
| ATOM | 3185 | N | PHE | B | 234 | 113.770 | 83.392 | 50.482 | 1.00 | 15.08 | N |
| ATOM | 3186 | CA | PHE | B | 234 | 114.552 | 83.382 | 49.247 | 1.00 | 13.98 | C |
| ATOM | 3187 | CB | PHE | B | 234 | 114.376 | 84.701 | 48.487 | 1.00 | 14.63 | C |
| ATOM | 3188 | CG | PHE | B | 234 | 112.977 | 84.909 | 47.954 | 1.00 | 13.38 | C |
| ATOM | 3189 | CD1 | PHE | B | 234 | 112.030 | 85.517 | 48.725 | 1.00 | 13.91 | C |
| ATOM | 3190 | CE1 | PHE | B | 234 | 110.747 | 85.674 | 48.273 | 1.00 | 14.13 | C |
| ATOM | 3191 | CZ | PHE | B | 234 | 110.408 | 85.243 | 47.006 | 1.00 | 13.32 | C |
| ATOM | 3192 | CE2 | PHE | B | 234 | 111.340 | 84.620 | 46.216 | 1.00 | 15.18 | C |
| ATOM | 3193 | CD2 | PHE | B | 234 | 112.625 | 84.451 | 46.691 | 1.00 | 15.65 | C |
| ATOM | 3194 | C | PHE | B | 234 | 116.038 | 83.167 | 49.478 | 1.00 | 14.23 | C |
| ATOM | 3195 | O | PHE | B | 234 | 116.808 | 83.290 | 48.525 | 1.00 | 13.61 | O |
| ATOM | 3196 | N | VAL | B | 235 | 116.439 | 82.813 | 50.703 | 1.00 | 14.20 | N |
| ATOM | 3197 | CA | VAL | B | 235 | 117.875 | 82.734 | 51.045 | 1.00 | 15.69 | C |
| ATOM | 3198 | CB | VAL | B | 235 | 118.087 | 82.656 | 52.599 | 1.00 | 16.11 | C |
| ATOM | 3199 | CG1 | VAL | B | 235 | 119.422 | 82.064 | 52.967 | 1.00 | 16.67 | C |
| ATOM | 3200 | CG2 | VAL | B | 235 | 117.938 | 84.005 | 53.207 | 1.00 | 15.96 | C |
| ATOM | 3201 | C | VAL | B | 235 | 118.632 | 81.641 | 50.280 | 1.00 | 15.68 | C |
| ATOM | 3202 | O | VAL | B | 235 | 119.719 | 81.915 | 49.755 | 1.00 | 15.19 | O |
| ATOM | 3203 | N | ASN | B | 236 | 118.067 | 80.442 | 50.155 | 1.00 | 16.48 | N |
| ATOM | 3204 | CA | ASN | B | 236 | 118.733 | 79.380 | 49.402 | 1.00 | 17.42 | C |
| ATOM | 3205 | CB | ASN | B | 236 | 117.935 | 78.070 | 49.450 | 1.00 | 18.24 | C |
| ATOM | 3206 | CG | ASN | B | 236 | 117.974 | 77.395 | 50.806 | 1.00 | 19.16 | C |
| ATOM | 3207 | OD1 | ASN | B | 236 | 117.106 | 76.542 | 51.102 | 1.00 | 24.07 | O |
| ATOM | 3208 | ND2 | ASN | B | 236 | 118.987 | 77.725 | 51.621 | 1.00 | 17.75 | N |
| ATOM | 3209 | C | ASN | B | 236 | 118.931 | 79.796 | 47.927 | 1.00 | 17.78 | C |
| ATOM | 3210 | O | ASN | B | 236 | 119.958 | 79.479 | 47.307 | 1.00 | 17.36 | O |
| ATOM | 3211 | N | TRP | B | 237 | 117.935 | 80.485 | 47.369 | 1.00 | 17.78 | N |
| ATOM | 3212 | CA | TRP | B | 237 | 118.026 | 80.996 | 45.987 | 1.00 | 16.95 | C |
| ATOM | 3213 | CB | TRP | B | 237 | 116.664 | 81.523 | 45.554 | 1.00 | 17.08 | C |
| ATOM | 3214 | CG | TRP | B | 237 | 116.574 | 82.142 | 44.207 | 1.00 | 15.83 | C |
| ATOM | 3215 | CD1 | TRP | B | 237 | 116.667 | 81.532 | 42.998 | 1.00 | 18.07 | C |
| ATOM | 3216 | NE1 | TRP | B | 237 | 116.463 | 82.451 | 41.999 | 1.00 | 15.62 | N |
| ATOM | 3217 | CE2 | TRP | B | 237 | 116.238 | 83.682 | 42.561 | 1.00 | 14.05 | C |
| ATOM | 3218 | CD2 | TRP | B | 237 | 116.285 | 83.512 | 43.950 | 1.00 | 14.17 | C |
| ATOM | 3219 | CE3 | TRP | B | 237 | 116.063 | 84.618 | 44.771 | 1.00 | 13.28 | C |
| ATOM | 3220 | CZ3 | TRP | B | 237 | 115.833 | 85.852 | 44.188 | 1.00 | 13.91 | C |
| ATOM | 3221 | CH2 | TRP | B | 237 | 115.769 | 85.984 | 42.779 | 1.00 | 12.38 | C |
| ATOM | 3222 | CZ2 | TRP | B | 237 | 115.986 | 84.914 | 41.964 | 1.00 | 13.15 | C |
| ATOM | 3223 | C | TRP | B | 237 | 119.091 | 82.077 | 45.859 | 1.00 | 16.55 | C |
| ATOM | 3224 | O | TRP | B | 237 | 119.954 | 82.020 | 44.967 | 1.00 | 16.15 | O |
| ATOM | 3225 | N | ILE | B | 238 | 119.073 | 83.039 | 46.773 | 1.00 | 15.75 | N |
| ATOM | 3226 | CA | ILE | B | 238 | 120.114 | 84.060 | 46.805 | 1.00 | 16.46 | C |
| ATOM | 3227 | CB | ILE | B | 238 | 119.913 | 85.017 | 48.003 | 1.00 | 16.12 | C |
| ATOM | 3228 | CG1 | ILE | B | 238 | 118.699 | 85.903 | 47.739 | 1.00 | 15.69 | C |
| ATOM | 3229 | CD1 | ILE | B | 238 | 118.081 | 86.463 | 48.978 | 1.00 | 15.32 | C |
| ATOM | 3230 | CG2 | ILE | B | 238 | 121.122 | 85.954 | 48.185 | 1.00 | 16.42 | C |
| ATOM | 3231 | C | ILE | B | 238 | 121.501 | 83.451 | 46.860 | 1.00 | 17.14 | C |
| ATOM | 3232 | O | ILE | B | 238 | 122.387 | 83.842 | 46.100 | 1.00 | 15.11 | O |
| ATOM | 3233 | N | ASP | B | 239 | 121.677 | 82.504 | 47.772 | 1.00 | 18.19 | N |
| ATOM | 3234 | CA | ASP | B | 239 | 122.960 | 81.805 | 47.921 | 1.00 | 19.76 | C |
| ATOM | 3235 | CB | ASP | B | 239 | 122.955 | 80.871 | 49.142 | 1.00 | 19.44 | C |
| ATOM | 3236 | CG | ASP | B | 239 | 122.955 | 81.624 | 50.464 | 1.00 | 19.09 | C |
| ATOM | 3237 | OD1 | ASP | B | 239 | 122.670 | 80.977 | 51.479 | 1.00 | 21.62 | O |
| ATOM | 3238 | OD2 | ASP | B | 239 | 123.192 | 82.852 | 50.603 | 1.00 | 15.68 | O |
| ATOM | 3239 | C | ASP | B | 239 | 123.307 | 80.987 | 46.687 | 1.00 | 19.86 | C |
| ATOM | 3240 | O | ASP | B | 239 | 124.457 | 80.766 | 46.453 | 1.00 | 21.99 | O |
| ATOM | 3241 | N | SER | B | 240 | 122.333 | 80.529 | 45.908 | 1.00 | 20.18 | N |
| ATOM | 3242 | CA | SER | B | 240 | 122.633 | 79.760 | 44.697 | 1.00 | 20.54 | C |
| ATOM | 3243 | CB | SER | B | 240 | 121.414 | 78.961 | 44.208 | 1.00 | 20.81 | C |
| ATOM | 3244 | OG | SER | B | 240 | 120.510 | 79.718 | 43.431 | 1.00 | 21.71 | O |
| ATOM | 3245 | C | SER | B | 240 | 123.181 | 80.665 | 43.596 | 1.00 | 20.47 | C |
| ATOM | 3246 | O | SER | B | 240 | 123.804 | 80.201 | 42.643 | 1.00 | 20.21 | O |
| ATOM | 3247 | N | ILE | B | 241 | 122.971 | 81.962 | 43.767 | 1.00 | 19.11 | N |
| ATOM | 3248 | CA | ILE | B | 241 | 123.358 | 82.952 | 42.788 | 1.00 | 19.49 | C |
| ATOM | 3249 | CB | ILE | B | 241 | 122.217 | 83.991 | 42.683 | 1.00 | 19.01 | C |
| ATOM | 3250 | CG1 | ILE | B | 241 | 120.975 | 83.314 | 42.059 | 1.00 | 16.33 | C |
| ATOM | 3251 | CD1 | ILE | B | 241 | 119.671 | 84.163 | 42.201 | 1.00 | 16.60 | C |
| ATOM | 3252 | CG2 | ILE | B | 241 | 122.683 | 85.223 | 41.946 | 1.00 | 16.85 | C |
| ATOM | 3253 | C | ILE | B | 241 | 124.671 | 83.605 | 43.206 | 1.00 | 21.09 | C |
| ATOM | 3254 | O | ILE | B | 241 | 125.575 | 83.664 | 42.430 | 1.00 | 20.34 | O |
| ATOM | 3255 | N | ILE | B | 242 | 124.744 | 84.020 | 44.463 | 1.00 | 23.05 | N |
| ATOM | 3256 | CA | ILE | B | 242 | 125.790 | 84.862 | 45.026 | 1.00 | 26.19 | C |
| ATOM | 3257 | CB | ILE | B | 242 | 125.140 | 85.581 | 46.229 | 1.00 | 26.53 | C |
| ATOM | 3258 | CG1 | ILE | B | 242 | 124.971 | 87.024 | 45.916 | 1.00 | 27.82 | C |
| ATOM | 3259 | CD1 | ILE | B | 242 | 123.864 | 81.172 | 45.067 | 1.00 | 28.44 | C |
| ATOM | 3260 | CG2 | ILE | B | 242 | 125.748 | 85.275 | 47.539 | 1.00 | 27.61 | C |
| ATOM | 3261 | C | ILE | B | 242 | 127.030 | 84.104 | 45.475 | 1.00 | 28.13 | C |
| ATOM | 3262 | O | ILE | B | 242 | 128.126 | 84.664 | 45.519 | 1.00 | 28.80 | O |
| ATOM | 3263 | N | GLN | B | 243 | 126.836 | 82.850 | 45.850 | 1.00 | 31.22 | N |
| ATOM | 3264 | CA | GLN | B | 243 | 127.947 | 81.954 | 46.211 | 1.00 | 33.85 | C |
| ATOM | 3265 | CB | GLN | B | 243 | 127.594 | 81.112 | 47.453 | 1.00 | 34.17 | C |
| ATOM | 3266 | CG | GLN | B | 243 | 126.925 | 81.888 | 48.600 | 1.00 | 36.39 | C |
| ATOM | 3267 | CD | GLN | B | 243 | 126.621 | 81.016 | 49.831 | 1.00 | 40.60 | C |
| ATOM | 3268 | OE1 | GLN | B | 243 | 126.835 | 79.786 | 49.825 | 1.00 | 40.55 | O |
| ATOM | 3269 | NE2 | GLN | B | 243 | 126.112 | 81.659 | 50.892 | 1.00 | 43.56 | N |
| ATOM | 3270 | C | GLN | B | 243 | 128.246 | 81.047 | 45.019 | 1.00 | 34.77 | C |
| ATOM | 3271 | O | GLN | B | 243 | 129.211 | 81.257 | 44.263 | 1.00 | 36.27 | O |
| ATOM | 3273 | C1 | NAG | A | 401 | 112.646 | 71.631 | 5.784 | 1.00 | 21.49 | C |
| ATOM | 3274 | C2 | NAG | A | 401 | 113.189 | 70.371 | 5.065 | 1.00 | 24.54 | C |
| ATOM | 3275 | N2 | NAG | A | 401 | 113.672 | 70.667 | 3.722 | 1.00 | 26.54 | N |
| ATOM | 3276 | C7 | NAG | A | 401 | 114.981 | 70.839 | 3.475 | 1.00 | 28.99 | C |
| ATOM | 3277 | O7 | NAG | A | 401 | 115.828 | 71.161 | 4.332 | 1.00 | 29.95 | O |
| ATOM | 3278 | C8 | NAG | A | 401 | 115.361 | 70.657 | 2.037 | 1.00 | 29.93 | C |
| ATOM | 3279 | C3 | NAG | A | 401 | 112.075 | 69.334 | 4.954 | 1.00 | 22.71 | C |
| ATOM | 3280 | O3 | NAG | A | 401 | 112.561 | 68.094 | 4.460 | 1.00 | 26.27 | O |
| ATOM | 3281 | C4 | NAG | A | 401 | 111.531 | 69.059 | 6.331 | 1.00 | 23.76 | C |
| ATOM | 3282 | O4 | NAG | A | 401 | 110.365 | 68.267 | 6.167 | 1.00 | 28.17 | O |
| ATOM | 3283 | C5 | NAG | A | 401 | 111.201 | 70.339 | 7.109 | 1.00 | 21.71 | C |
| ATOM | 3284 | C6 | NAG | A | 401 | 110.898 | 69.955 | 8.562 | 1.00 | 20.46 | C |
| ATOM | 3285 | O6 | NAG | A | 401 | 112.090 | 69.691 | 9.265 | 1.00 | 22.34 | O |
| ATOM | 3286 | O5 | NAG | A | 401 | 112.341 | 71.172 | 7.089 | 1.00 | 17.83 | O |
| ATOM | 3287 | C1 | FUC | A | 402 | 111.852 | 68.652 | 10.260 | 1.00 | 24.14 | C |
| ATOM | 3288 | C2 | FUC | A | 402 | 113.170 | 68.387 | 10.977 | 1.00 | 24.78 | C |
| ATOM | 3289 | O2 | FUC | A | 402 | 114.229 | 68.248 | 10.020 | 1.00 | 26.59 | O |
| ATOM | 3290 | C3 | FUC | A | 402 | 113.519 | 69.548 | 11.904 | 1.00 | 22.03 | C |
| ATOM | 3291 | O3 | FUC | A | 402 | 114.709 | 69.324 | 12.645 | 1.00 | 24.14 | O |
| ATOM | 3292 | C4 | FUC | A | 402 | 112.317 | 69.921 | 12.777 | 1.00 | 24.17 | C |
| ATOM | 3293 | O4 | FUC | A | 402 | 112.002 | 68.935 | 13.747 | 1.00 | 20.21 | O |
| ATOM | 3294 | C5 | FUC | A | 402 | 111.110 | 70.207 | 11.893 | 1.00 | 22.63 | C |
| ATOM | 3295 | C6 | FUC | A | 402 | 109.888 | 70.657 | 12.695 | 1.00 | 22.90 | C |
| ATOM | 3296 | O5 | FUC | A | 402 | 110.806 | 69.042 | 11.147 | 1.00 | 25.84 | O |
| ATOM | 3297 | C1 | NAG | A | 403 | 110.916 | 66.654 | 6.894 | 1.00 | 42.52 | C |
| ATOM | 3298 | C2 | NAG | A | 403 | 109.522 | 66.124 | 7.161 | 1.00 | 41.55 | C |
| ATOM | 3299 | N2 | NAG | A | 403 | 109.108 | 66.661 | 8.448 | 1.00 | 38.67 | N |
| ATOM | 3300 | C7 | NAG | A | 403 | 108.118 | 67.531 | 8.604 | 1.00 | 36.56 | C |
| ATOM | 3301 | O7 | NAG | A | 403 | 107.344 | 67.853 | 7.698 | 1.00 | 33.37 | O |
| ATOM | 3302 | C8 | NAG | A | 403 | 107.977 | 68.120 | 9.986 | 1.00 | 36.51 | C |
| ATOM | 3303 | C3 | NAG | A | 403 | 109.513 | 64.600 | 7.196 | 1.00 | 42.70 | C |
| ATOM | 3304 | O3 | NAG | A | 403 | 108.160 | 64.183 | 7.072 | 1.00 | 40.14 | O |
| ATOM | 3305 | C4 | NAG | A | 403 | 110.427 | 63.976 | 6.132 | 1.00 | 44.40 | C |
| ATOM | 3306 | O4 | NAG | A | 403 | 110.766 | 62.680 | 6.568 | 1.00 | 45.80 | O |
| ATOM | 3307 | C5 | NAG | A | 403 | 111.734 | 64.749 | 5.885 | 1.00 | 44.72 | C |
| ATOM | 3308 | C6 | NAG | A | 403 | 112.497 | 64.258 | 4.654 | 1.00 | 45.16 | C |
| ATOM | 3309 | O6 | NAG | A | 403 | 111.800 | 64.625 | 3.484 | 1.00 | 45.00 | O |
| ATOM | 3310 | O5 | NAG | A | 403 | 111.438 | 66.119 | 5.704 | 1.00 | 44.28 | O |
| ATOM | 3311 | C1 | NAG | A | 411 | 93.334 | 85.968 | 32.276 | 1.00 | 28.84 | C |
| ATOM | 3312 | C2 | NAG | A | 411 | 91.904 | 85.632 | 32.734 | 1.00 | 32.65 | C |
| ATOM | 3313 | N2 | NAG | A | 411 | 91.757 | 84.176 | 32.889 | 1.00 | 32.08 | N |
| ATOM | 3314 | C7 | NAG | A | 411 | 91.631 | 83.591 | 34.082 | 1.00 | 33.79 | C |
| ATOM | 3315 | O7 | NAG | A | 411 | 91.581 | 84.232 | 35.130 | 1.00 | 35.33 | O |
| ATOM | 3316 | C8 | NAG | A | 411 | 91.562 | 82.097 | 34.154 | 1.00 | 34.29 | C |
| ATOM | 3317 | C3 | NAG | A | 411 | 90.825 | 86.087 | 31.741 | 1.00 | 35.93 | C |
| ATOM | 3318 | O3 | NAG | A | 411 | 89.585 | 86.234 | 32.409 | 1.00 | 36.78 | O |
| ATOM | 3319 | C4 | NAG | A | 411 | 91.129 | 87.377 | 30.979 | 1.00 | 37.32 | C |
| ATOM | 3320 | O4 | NAG | A | 411 | 90.242 | 87.395 | 29.868 | 1.00 | 38.92 | O |
| ATOM | 3321 | C5 | NAG | A | 411 | 92.595 | 87.440 | 30.549 | 1.00 | 35.74 | C |
| ATOM | 3322 | C6 | NAG | A | 411 | 92.924 | 88.746 | 29.811 | 1.00 | 37.22 | C |
| ATOM | 3323 | O6 | NAG | A | 411 | 92.998 | 89.885 | 30.654 | 1.00 | 37.12 | O |
| ATOM | 3324 | O5 | NAG | A | 411 | 93.413 | 87.269 | 31.707 | 1.00 | 32.50 | O |
| ATOM | 3325 | C1 | NAG | B | 401 | 114.705 | 102.353 | 67.106 | 1.00 | 19.27 | C |
| ATOM | 3326 | C2 | NAG | B | 401 | 114.714 | 101.921 | 68.584 | 1.00 | 17.70 | C |
| ATOM | 3327 | N2 | NAG | B | 401 | 113.836 | 102.786 | 69.350 | 1.00 | 16.49 | N |
| ATOM | 3328 | C7 | NAG | B | 401 | 112.564 | 102.553 | 69.628 | 1.00 | 17.35 | C |
| ATOM | 3329 | O7 | NAG | B | 401 | 111.875 | 103.338 | 70.325 | 1.00 | 26.16 | O |
| ATOM | 3330 | C8 | NAG | B | 401 | 111.931 | 101.371 | 69.045 | 1.00 | 14.58 | C |
| ATOM | 3331 | C3 | NAG | B | 401 | 116.121 | 102.006 | 69.180 | 1.00 | 19.15 | C |
| ATOM | 3332 | O3 | NAG | B | 401 | 116.122 | 101.440 | 70.480 | 1.00 | 17.74 | O |
| ATOM | 3333 | C4 | NAG | B | 401 | 117.089 | 101.260 | 68.278 | 1.00 | 19.42 | C |
| ATOM | 3334 | O4 | NAG | B | 401 | 118.437 | 101.510 | 68.639 | 1.00 | 21.79 | O |
| ATOM | 3335 | C5 | NAG | B | 401 | 116.900 | 101.601 | 66.808 | 1.00 | 19.20 | C |
| ATOM | 3336 | C6 | NAG | B | 401 | 117.687 | 100.608 | 65.959 | 1.00 | 16.84 | C |
| ATOM | 3337 | O6 | NAG | B | 401 | 117.080 | 99.331 | 66.054 | 1.00 | 17.18 | O |
| ATOM | 3338 | O5 | NAG | B | 401 | 115.545 | 101.462 | 66.427 | 1.00 | 15.34 | O |
| ATOM | 3339 | C1 | FUC | B | 402 | 118.026 | 98.271 | 66.125 | 1.00 | 17.34 | C |
| ATOM | 3340 | C2 | FUC | B | 402 | 117.258 | 96.962 | 66.126 | 1.00 | 17.76 | C |
| ATOM | 3341 | O2 | FUC | B | 402 | 116.260 | 97.076 | 67.124 | 1.00 | 20.31 | O |
| ATOM | 3342 | C3 | FUC | B | 402 | 116.571 | 96.695 | 64.792 | 1.00 | 13.01 | C |
| ATOM | 3343 | O3 | FUC | B | 402 | 116.015 | 95.394 | 64.843 | 1.00 | 15.39 | O |
| ATOM | 3344 | C4 | FUC | B | 402 | 117.607 | 96.841 | 63.678 | 1.00 | 14.68 | C |
| ATOM | 3345 | O4 | FUC | B | 402 | 118.598 | 95.872 | 63.828 | 1.00 | 14.17 | O |
| ATOM | 3346 | C5 | FUC | B | 402 | 118.272 | 98.194 | 63.764 | 1.00 | 16.97 | C |
| ATOM | 3347 | C6 | FUC | B | 402 | 119.281 | 98.434 | 62.650 | 1.00 | 18.93 | C |
| ATOM | 3348 | O5 | FUC | B | 402 | 118.925 | 98.348 | 65.040 | 1.00 | 18.61 | O |
| ATOM | 3349 | C1 | NAG | B | 403 | 119.039 | 100.512 | 69.828 | 1.00 | 28.21 | C |
| ATOM | 3350 | C2 | NAG | B | 403 | 120.541 | 100.648 | 69.584 | 1.00 | 29.36 | C |
| ATOM | 3351 | N2 | NAG | B | 403 | 120.887 | 99.991 | 68.332 | 1.00 | 28.31 | N |
| ATOM | 3352 | C7 | NAG | B | 403 | 121.295 | 100.691 | 67.272 | 1.00 | 31.08 | C |
| ATOM | 3353 | O7 | NAG | B | 403 | 121.374 | 101.919 | 67.270 | 1.00 | 30.96 | O |
| ATOM | 3354 | C8 | NAG | B | 403 | 121.650 | 99.929 | 66.023 | 1.00 | 32.05 | C |
| ATOM | 3355 | C3 | NAG | B | 403 | 121.298 | 100.071 | 70.767 | 1.00 | 31.22 | C |
| ATOM | 3356 | O3 | NAG | B | 403 | 122.659 | 100.378 | 70.583 | 1.00 | 30.04 | O |
| ATOM | 3357 | C4 | NAG | B | 403 | 120.747 | 100.683 | 72.061 | 1.00 | 30.44 | C |
| ATOM | 3358 | O4 | NAG | B | 403 | 121.350 | 100.010 | 73.159 | 1.00 | 34.30 | O |
| ATOM | 3359 | C5 | NAG | B | 403 | 119.202 | 100.637 | 72.113 | 1.00 | 30.11 | C |
| ATOM | 3360 | C6 | NAG | B | 403 | 118.593 | 101.383 | 73.284 | 1.00 | 31.08 | C |
| ATOM | 3361 | O6 | NAG | B | 403 | 118.907 | 102.748 | 73.074 | 1.00 | 34.53 | O |
| ATOM | 3362 | O5 | NAG | B | 403 | 118.650 | 101.256 | 70.956 | 1.00 | 25.75 | O |
| ATOM | 3363 | C1 | NAG | B | 411 | 130.453 | 99.658 | 35.277 | 1.00 | 27.32 | C |
| ATOM | 3364 | C2 | NAG | B | 411 | 131.840 | 99.952 | 34.653 | 1.00 | 29.74 | C |
| ATOM | 3365 | N2 | NAG | B | 411 | 132.854 | 99.050 | 35.181 | 1.00 | 30.54 | N |
| ATOM | 3366 | C7 | NAG | B | 411 | 133.476 | 98.110 | 34.468 | 1.00 | 32.83 | C |
| ATOM | 3367 | O7 | NAG | B | 411 | 133.243 | 97.850 | 33.286 | 1.00 | 35.18 | O |
| ATOM | 3368 | C8 | NAG | B | 411 | 134.512 | 97.305 | 35.200 | 1.00 | 34.71 | C |
| ATOM | 3369 | C3 | NAG | B | 411 | 132.281 | 101.389 | 34.961 | 1.00 | 31.42 | C |
| ATOM | 3370 | O3 | NAG | B | 411 | 133.395 | 101.757 | 34.170 | 1.00 | 35.40 | O |
| ATOM | 3371 | C4 | NAG | B | 411 | 131.200 | 102.405 | 34.662 | 1.00 | 31.20 | C |
| ATOM | 3372 | O4 | NAG | B | 411 | 131.615 | 103.639 | 35.221 | 1.00 | 31.13 | O |
| ATOM | 3373 | C5 | NAG | B | 411 | 129.832 | 101.950 | 35.198 | 1.00 | 27.91 | C |
| ATOM | 3374 | C6 | NAG | B | 411 | 128.743 | 102.952 | 34.812 | 1.00 | 28.00 | C |
| ATOM | 3375 | O6 | NAG | B | 411 | 128.453 | 102.991 | 33.419 | 1.00 | 27.09 | O |
| ATOM | 3376 | O5 | NAG | B | 411 | 129.532 | 100.624 | 34.774 | 1.00 | 28.14 | O |
| ATOM | 3377 | S | SO4 | S | 1 | 113.325 | 92.041 | 18.417 | 1.00 | 23.89 | S |
| ATOM | 3378 | O1 | SO4 | S | 1 | 114.690 | 92.543 | 18.468 | 1.00 | 25.09 | O |
| ATOM | 3379 | O2 | SO4 | S | 1 | 112.397 | 93.016 | 17.872 | 1.00 | 24.71 | O |
| ATOM | 3380 | O3 | SO4 | S | 1 | 112.970 | 91.764 | 19.809 | 1.00 | 24.35 | O |
| ATOM | 3381 | O4 | SO4 | S | 1 | 113.339 | 90.825 | 17.612 | 1.00 | 24.83 | O |
| ATOM | 3382 | S | SO4 | S | 2 | 104.651 | 99.725 | 26.842 | 1.00 | 49.24 | S |
| ATOM | 3383 | O1 | SO4 | S | 2 | 105.906 | 100.492 | 27.007 | 1.00 | 49.72 | O |
| ATOM | 3384 | O2 | SO4 | S | 2 | 103.527 | 100.501 | 27.366 | 1.00 | 49.47 | O |
| ATOM | 3385 | O3 | SO4 | S | 2 | 104.681 | 98.449 | 27.521 | 1.00 | 45.08 | O |
| ATOM | 3386 | O4 | SO4 | S | 2 | 104.464 | 99.476 | 25.413 | 1.00 | 49.42 | O |
| ATOM | 3387 | S | SO4 | S | 3 | 117.224 | 65.616 | 20.011 | 1.00 | 42.53 | S |
| ATOM | 3388 | O1 | SO4 | S | 3 | 116.238 | 64.552 | 20.210 | 1.00 | 42.36 | O |
| ATOM | 3389 | O2 | SO4 | S | 3 | 116.570 | 66.764 | 19.331 | 1.00 | 40.18 | O |
| ATOM | 3390 | O3 | SO4 | S | 3 | 118.275 | 65.026 | 19.165 | 1.00 | 42.50 | O |
| ATOM | 3391 | O4 | SO4 | S | 3 | 117.834 | 66.085 | 21.253 | 1.00 | 36.96 | O |
| ATOM | 3392 | S | SO4 | S | 4 | 110.589 | 86.206 | −1.660 | 1.00 | 52.65 | S |
| ATOM | 3393 | O1 | SO4 | S | 4 | 109.191 | 86.582 | −1.412 | 1.00 | 53.10 | O |
| ATOM | 3394 | O2 | SO4 | S | 4 | 111.010 | 86.477 | −3.033 | 1.00 | 51.12 | O |
| ATOM | 3395 | O3 | SO4 | S | 4 | 110.729 | 84.770 | −1.427 | 1.00 | 52.17 | O |
| ATOM | 3396 | O4 | SO4 | S | 4 | 111.425 | 86.973 | −0.722 | 1.00 | 50.93 | O |
| ATOM | 3397 | S | SO4 | S | 5 | 102.028 | 86.833 | 4.697 | 1.00 | 35.87 | S |
| ATOM | 3398 | O1 | SO4 | S | 5 | 100.557 | 86.815 | 4.559 | 1.00 | 37.44 | O |
| ATOM | 3399 | O2 | SO4 | S | 5 | 102.565 | 88.003 | 3.967 | 1.00 | 38.24 | O |
| ATOM | 3400 | O3 | SO4 | S | 5 | 102.708 | 85.641 | 4.108 | 1.00 | 30.08 | O |
| ATOM | 3401 | O4 | SO4 | S | 5 | 102.318 | 87.088 | 6.074 | 1.00 | 28.71 | O |
| ATOM | 3402 | S | SO4 | S | 6 | 107.373 | 102.618 | 44.369 | 1.00 | 27.28 | S |
| ATOM | 3403 | O1 | SO4 | S | 6 | 106.017 | 102.078 | 44.242 | 1.00 | 28.16 | O |
| ATOM | 3404 | O2 | SO4 | S | 6 | 107.369 | 104.033 | 43.942 | 1.00 | 23.92 | O |
| ATOM | 3405 | O3 | SO4 | S | 6 | 108.326 | 101.835 | 43.571 | 1.00 | 21.37 | O |
| ATOM | 3406 | O4 | SO4 | S | 6 | 107.662 | 102.700 | 45.801 | 1.00 | 27.08 | O |
| ATOM | 3407 | S | SO4 | S | 7 | 117.198 | 86.710 | 63.462 | 1.00 | 26.00 | S |
| ATOM | 3408 | O1 | SO4 | S | 7 | 118.600 | 86.530 | 63.875 | 1.00 | 25.85 | O |
| ATOM | 3409 | O2 | SO4 | S | 7 | 117.097 | 88.114 | 63.017 | 1.00 | 24.46 | O |
| ATOM | 3410 | O3 | SO4 | S | 7 | 116.335 | 86.535 | 64.662 | 1.00 | 25.23 | O |
| ATOM | 3411 | O4 | SO4 | S | 7 | 116.817 | 85.721 | 62.416 | 1.00 | 25.66 | O |
| ATOM | 3412 | S | SO4 | S | 8 | 115.657 | 115.787 | 54.273 | 1.00 | 37.82 | S |
| ATOM | 3413 | O1 | SO4 | S | 8 | 116.962 | 116.244 | 53.815 | 1.00 | 40.58 | O |
| ATOM | 3414 | O2 | SO4 | S | 8 | 114.684 | 116.874 | 54.162 | 1.00 | 41.14 | O |
| ATOM | 3415 | O3 | SO4 | S | 8 | 115.724 | 115.358 | 55.678 | 1.00 | 35.53 | O |
| ATOM | 3416 | O4 | SO4 | S | 8 | 115.246 | 114.652 | 53.450 | 1.00 | 41.39 | O |
| ATOM | 3417 | S | SO4 | S | 9 | 103.443 | 85.274 | −2.147 | 1.00 | 61.31 | S |
| ATOM | 3418 | O1 | SO4 | S | 9 | 103.549 | 85.933 | −3.456 | 1.00 | 59.80 | O |
| ATOM | 3419 | O2 | SO4 | S | 9 | 104.749 | 84.817 | −1.664 | 1.00 | 60.62 | O |
| ATOM | 3420 | O3 | SO4 | S | 9 | 102.541 | 84.123 | −2.274 | 1.00 | 61.06 | O |
| ATOM | 3421 | O4 | SO4 | S | 9 | 102.877 | 86.214 | −1.176 | 1.00 | 61.49 | O |
| ATOM | 3422 | S | SO4 | S | 10 | 105.427 | 100.524 | 35.567 | 1.00 | 87.02 | S |
| ATOM | 3423 | O1 | SO4 | S | 10 | 106.545 | 101.100 | 36.315 | 1.00 | 87.77 | O |
| ATOM | 3424 | O2 | SO4 | S | 10 | 105.312 | 101.197 | 34.272 | 1.00 | 86.97 | O |
| ATOM | 3425 | O3 | SO4 | S | 10 | 104.203 | 100.723 | 36.335 | 1.00 | 86.75 | O |
| ATOM | 3426 | O4 | SO4 | S | 10 | 105.662 | 99.097 | 35.369 | 1.00 | 86.77 | O |
| ATOM | 3427 | S | SO4 | S | 11 | 127.639 | 119.895 | 46.466 | 1.00 | 36.45 | S |
| ATOM | 3428 | O1 | SO4 | S | 11 | 128.433 | 119.077 | 47.359 | 1.00 | 36.11 | O |
| ATOM | 3429 | O2 | SO4 | S | 11 | 128.521 | 120.905 | 45.877 | 1.00 | 39.85 | O |
| ATOM | 3430 | O3 | SO4 | S | 11 | 126.574 | 120.611 | 47.154 | 1.00 | 38.33 | O |
| ATOM | 3431 | O4 | SO4 | S | 11 | 127.114 | 119.074 | 45.387 | 1.00 | 37.16 | O |
| ATOM | 3432 | S | SO4 | S | 12 | 98.556 | 84.229 | 52.046 | 1.00 | 64.84 | S |
| ATOM | 3433 | O1 | SO4 | S | 12 | 97.662 | 84.830 | 51.053 | 1.00 | 62.73 | O |
| ATOM | 3434 | O2 | SO4 | S | 12 | 99.933 | 84.481 | 51.629 | 1.00 | 62.43 | O |
| ATOM | 3435 | O3 | SO4 | S | 12 | 98.387 | 82.773 | 52.155 | 1.00 | 63.84 | O |
| ATOM | 3436 | O4 | SO4 | S | 12 | 98.223 | 84.822 | 53.351 | 1.00 | 62.41 | O |
| ATOM | 3437 | O | HOH | W | 1 | 110.117 | 80.433 | 23.068 | 1.00 | 10.39 | O |
| ATOM | 3438 | O | HOH | W | 2 | 107.200 | 85.752 | 16.195 | 1.00 | 8.26 | O |
| ATOM | 3439 | O | HOH | W | 3 | 107.741 | 86.258 | 9.894 | 1.00 | 7.31 | O |
| ATOM | 3440 | O | HOH | W | 4 | 128.967 | 84.245 | 4.890 | 1.00 | 12.55 | O |
| ATOM | 3441 | O | HOH | W | 5 | 121.128 | 85.283 | 23.466 | 1.00 | 11.38 | O |
| ATOM | 3442 | O | HOH | W | 6 | 112.841 | 100.236 | 54.086 | 1.00 | 9.14 | O |
| ATOM | 3443 | O | HOH | W | 7 | 107.230 | 90.004 | 47.894 | 1.00 | 10.22 | O |
| ATOM | 3444 | O | HOH | W | 8 | 105.304 | 91.886 | 47.940 | 1.00 | 8.49 | O |
| ATOM | 3445 | O | HOH | W | 9 | 118.411 | 86.901 | 9.987 | 1.00 | 10.28 | O |
| ATOM | 3446 | O | HOH | W | 10 | 104.739 | 99.833 | 45.233 | 1.00 | 19.39 | O |
| ATOM | 3447 | O | HOH | W | 11 | 124.002 | 104.403 | 48.478 | 1.00 | 10.72 | O |
| ATOM | 3448 | O | HOH | W | 12 | 103.249 | 103.616 | 54.599 | 1.00 | 11.64 | O |
| ATOM | 3449 | O | HOH | W | 13 | 110.726 | 87.294 | 2.931 | 1.00 | 14.20 | O |
| ATOM | 3450 | O | HOH | W | 14 | 122.527 | 99.269 | 34.378 | 1.00 | 17.18 | O |
| ATOM | 3451 | O | HOH | W | 15 | 100.648 | 100.348 | 53.905 | 1.00 | 9.05 | O |
| ATOM | 3452 | O | HOH | W | 16 | 121.650 | 84.703 | 9.496 | 1.00 | 8.98 | O |
| ATOM | 3453 | O | HOH | W | 17 | 99.517 | 81.667 | 18.876 | 1.00 | 8.68 | O |
| ATOM | 3454 | O | HOH | W | 18 | 112.207 | 80.978 | 15.365 | 1.00 | 10.30 | O |
| ATOM | 3455 | O | HOH | W | 19 | 120.638 | 83.547 | 25.532 | 1.00 | 13.06 | O |
| ATOM | 3456 | O | HOH | W | 20 | 111.222 | 87.456 | 41.943 | 1.00 | 11.25 | O |
| ATOM | 3457 | O | HOH | W | 21 | 112.587 | 109.014 | 52.693 | 1.00 | 12.52 | O |
| ATOM | 3458 | O | HOH | W | 22 | 104.633 | 89.777 | 58.892 | 1.00 | 13.98 | O |
| ATOM | 3459 | O | HOH | W | 23 | 113.031 | 77.088 | 4.086 | 1.00 | 13.01 | O |
| ATOM | 3460 | O | HOH | W | 24 | 99.980 | 90.459 | 31.295 | 1.00 | 12.74 | O |
| ATOM | 3461 | O | HOH | W | 25 | 123.753 | 104.742 | 55.025 | 1.00 | 20.55 | O |
| ATOM | 3462 | O | HOH | W | 26 | 90.537 | 97.070 | 53.056 | 1.00 | 13.64 | O |
| ATOM | 3463 | O | HOH | W | 27 | 99.259 | 78.670 | 13.103 | 1.00 | 13.39 | O |
| ATOM | 3464 | O | HOH | W | 28 | 109.231 | 84.432 | 5.143 | 1.00 | 11.45 | O |
| ATOM | 3465 | O | HOH | W | 29 | 122.123 | 87.069 | 12.126 | 1.00 | 10.19 | O |
| ATOM | 3466 | O | HOH | W | 30 | 99.811 | 78.766 | 16.756 | 1.00 | 12.04 | O |
| ATOM | 3467 | O | HOH | W | 31 | 117.106 | 84.575 | 32.167 | 1.00 | 11.72 | O |
| ATOM | 3468 | O | HOH | W | 32 | 118.364 | 91.466 | 16.737 | 1.00 | 16.10 | O |
| ATOM | 3469 | O | HOH | W | 33 | 119.655 | 74.158 | 23.379 | 1.00 | 14.42 | O |
| ATOM | 3470 | O | HOH | W | 34 | 95.981 | 90.912 | 22.237 | 1.00 | 18.30 | O |
| ATOM | 3471 | O | HOH | W | 35 | 122.075 | 88.252 | 9.545 | 1.00 | 9.63 | O |
| ATOM | 3472 | O | HOH | W | 36 | 116.881 | 95.355 | 49.114 | 1.00 | 9.00 | O |
| ATOM | 3473 | O | HOH | W | 37 | 99.368 | 102.600 | 54.527 | 1.00 | 12.43 | O |
| ATOM | 3474 | O | HOH | W | 38 | 103.910 | 74.211 | 7.827 | 1.00 | 12.51 | O |
| ATOM | 3475 | O | HOH | W | 39 | 97.547 | 93.006 | 23.583 | 1.00 | 17.13 | O |
| ATOM | 3476 | O | HOH | W | 40 | 113.209 | 91.129 | 30.989 | 1.00 | 14.06 | O |
| ATOM | 3477 | O | HOH | W | 41 | 129.534 | 75.437 | 9.882 | 1.00 | 9.50 | O |
| ATOM | 3478 | O | HOH | W | 42 | 115.193 | 80.254 | 48.599 | 1.00 | 15.45 | O |
| ATOM | 3479 | O | HOH | W | 43 | 120.221 | 82.616 | 8.415 | 1.00 | 12.09 | O |
| ATOM | 3480 | O | HOH | W | 44 | 132.717 | 91.628 | 11.418 | 1.00 | 22.38 | O |
| ATOM | 3481 | O | HOH | W | 45 | 118.883 | 81.329 | 10.483 | 1.00 | 18.02 | O |
| ATOM | 3482 | O | HOH | W | 46 | 128.499 | 82.967 | 8.789 | 1.00 | 19.64 | O |
| ATOM | 3483 | O | HOH | W | 47 | 103.284 | 100.996 | 46.982 | 1.00 | 17.86 | O |
| ATOM | 3484 | O | H0H | W | 48 | 105.917 | 100.462 | 58.368 | 1.00 | 12.36 | O |
| ATOM | 3485 | O | HOH | W | 49 | 112.486 | 93.071 | 68.722 | 1.00 | 18.07 | O |
| ATOM | 3486 | O | HOH | W | 50 | 103.487 | 101.848 | 59.306 | 1.00 | 15.35 | O |
| ATOM | 3487 | O | HOH | W | 51 | 122.868 | 82.349 | 26.879 | 1.00 | 11.65 | O |
| ATOM | 3488 | O | HOH | W | 52 | 121.099 | 75.820 | 5.302 | 1.00 | 19.92 | O |
| ATOM | 3489 | O | HOH | W | 53 | 111.231 | 93.183 | 36.834 | 1.00 | 14.77 | O |
| ATOM | 3490 | O | HOH | W | 54 | 112.176 | 87.504 | 55.724 | 1.00 | 16.72 | O |
| ATOM | 3491 | O | HOH | W | 55 | 114.838 | 104.311 | 48.774 | 1.00 | 13.01 | O |
| ATOM | 3492 | O | HOH | W | 56 | 101.648 | 101.315 | 57.229 | 1.00 | 10.55 | o |
| ATOM | 3493 | O | HOH | W | 57 | 118.936 | 74.489 | 34.014 | 1.00 | 13.20 | O |
| ATOM | 3494 | O | HOH | W | 58 | 113.590 | 99.296 | 1.638 | 1.00 | 49.47 | O |
| ATOM | 3495 | O | HOH | W | 59 | 101.226 | 104.235 | 61.205 | 1.00 | 14.88 | O |
| ATOM | 3496 | O | HOH | W | 60 | 101.056 | 77.084 | 14.288 | 1.00 | 14.42 | O |
| ATOM | 3497 | O | HOH | W | 61 | 110.946 | 110.841 | 57.273 | 1.00 | 14.88 | O |
| ATOM | 3498 | O | HOH | W | 62 | 121.586 | 85.396 | 6.760 | 1.00 | 13.16 | O |
| ATOM | 3499 | O | HOH | W | 63 | 99.466 | 78.216 | 20.519 | 1.00 | 11.31 | O |
| ATOM | 3500 | O | HOH | W | 64 | 100.541 | 91.799 | 52.780 | 1.00 | 16.47 | O |
| ATOM | 3501 | O | HOH | W | 65 | 122.023 | 83.251 | 5.111 | 1.00 | 15.02 | O |
| ATOM | 3502 | O | HOH | W | 66 | 100.704 | 103.625 | 58.407 | 1.00 | 15.14 | O |
| ATOM | 3503 | O | HOH | W | 67 | 111.153 | 106.131 | 64.335 | 1.00 | 15.36 | O |
| ATOM | 3504 | O | HOH | W | 68 | 124.485 | 104.361 | 51.914 | 1.00 | 14.56 | O |
| ATOM | 3505 | O | HOH | W | 69 | 128.117 | 80.684 | 10.295 | 1.00 | 15.71 | O |
| ATOM | 3506 | O | HOH | W | 70 | 106.260 | 87.098 | 48.345 | 1.00 | 14.22 | O |
| ATOM | 3507 | O | HOH | W | 71 | 97.877 | 76.930 | 16.561 | 1.00 | 13.34 | O |
| ATOM | 3508 | O | HOH | W | 72 | 123.182 | 93.475 | 57.675 | 1.00 | 17.18 | O |
| ATOM | 3509 | O | HOH | W | 73 | 120.305 | 88.337 | 64.380 | 1.00 | 30.10 | O |
| ATOM | 3510 | O | HOH | W | 74 | 124.661 | 89.058 | 51.981 | 1.00 | 19.33 | O |
| ATOM | 3511 | O | HOH | W | 75 | 127.060 | 104.745 | 52.973 | 1.00 | 18.65 | O |
| ATOM | 3512 | O | HOH | W | 76 | 109.337 | 94.680 | 32.404 | 1.00 | 23.83 | O |
| ATOM | 3513 | O | HOH | W | 77 | 107.635 | 112.833 | 57.201 | 1.00 | 24.23 | O |
| ATOM | 3514 | O | HOH | W | 78 | 125.346 | 76.594 | 17.584 | 1.00 | 11.64 | O |
| ATOM | 3515 | O | HOH | W | 79 | 121.191 | 92.429 | 6.559 | 1.00 | 17.98 | O |
| ATOM | 3516 | O | HOH | W | 80 | 124.514 | 74.023 | 18.996 | 1.00 | 24.66 | O |
| ATOM | 3517 | O | HOH | W | 81 | 132.669 | 80.453 | 16.104 | 1.00 | 24.34 | O |
| ATOM | 3518 | O | HOH | W | 82 | 91.201 | 79.093 | 27.249 | 1.00 | 26.33 | O |
| ATOM | 3519 | O | HOH | W | 83 | 114.868 | 94.906 | 25.620 | 1.00 | 26.02 | O |
| ATOM | 3520 | O | HOH | W | 84 | 128.339 | 67.960 | 14.205 | 1.00 | 16.32 | O |
| ATOM | 3521 | O | HOH | W | 85 | 99.974 | 108.355 | 49.323 | 1.00 | 31.30 | O |
| ATOM | 3522 | O | HOH | W | 86 | 117.594 | 91.423 | 19.322 | 1.00 | 28.01 | O |
| ATOM | 3523 | O | HOH | W | 87 | 115.175 | 92.151 | 2.773 | 1.00 | 32.53 | O |
| ATOM | 3524 | O | HOH | W | 88 | 104.746 | 75.343 | 5.352 | 1.00 | 23.21 | O |
| ATOM | 3525 | O | HOH | W | 89 | 116.799 | 94.186 | 27.596 | 1.00 | 19.47 | O |
| ATOM | 3526 | O | HOH | W | 90 | 91.568 | 76.947 | 21.016 | 1.00 | 15.21 | O |
| ATOM | 3527 | O | HOH | W | 91 | 113.376 | 113.994 | 58.476 | 1.00 | 30.25 | O |
| ATOM | 3528 | O | HOH | W | 92 | 126.042 | 101.512 | 49.757 | 1.00 | 15.44 | O |
| ATOM | 3529 | O | HOH | W | 93 | 121.174 | 106.571 | 62.070 | 1.00 | 19.09 | O |
| ATOM | 3530 | O | HOH | W | 94 | 107.643 | 65.244 | 36.988 | 1.00 | 20.49 | O |
| ATOM | 3531 | O | HOH | W | 95 | 103.159 | 67.933 | 23.440 | 1.00 | 17.11 | O |
| ATOM | 3532 | O | HOH | W | 96 | 114.740 | 93.416 | 31.877 | 1.00 | 23.65 | O |
| ATOM | 3533 | O | HOH | W | 97 | 105.603 | 102.255 | 48.040 | 1.00 | 24.95 | O |
| ATOM | 3534 | O | HOH | W | 98 | 100.503 | 87.068 | 58.063 | 1.00 | 24.86 | O |
| ATOM | 3535 | O | HOH | W | 99 | 109.518 | 78.908 | 42.118 | 1.00 | 33.74 | O |
| ATOM | 3536 | O | HOH | W | 100 | 107.230 | 100.115 | 49.113 | 1.00 | 17.36 | O |
| ATOM | 3537 | O | HOH | W | 101 | 124.851 | 91.575 | 4.324 | 1.00 | 22.94 | O |
| ATOM | 3538 | O | HOH | W | 102 | 98.591 | 79.838 | 36.573 | 1.00 | 22.18 | O |
| ATOM | 3539 | O | HOH | W | 103 | 103.095 | 87.854 | 57.476 | 1.00 | 15.12 | O |
| ATOM | 3540 | O | HOH | W | 104 | 137.371 | 103.022 | 54.874 | 1.00 | 35.35 | O |
| ATOM | 3541 | O | HOH | W | 105 | 118.278 | 84.024 | 60.893 | 1.00 | 20.67 | O |
| ATOM | 3542 | O | HOH | W | 106 | 135.667 | 94.713 | 39.485 | 1.00 | 34.27 | O |
| ATOM | 3543 | O | HOH | W | 107 | 108.639 | 80.862 | 44.327 | 1.00 | 28.68 | O |
| ATOM | 3544 | O | HOH | W | 108 | 123.139 | 81.278 | 35.018 | 1.00 | 25.75 | O |
| ATOM | 3545 | O | HOH | W | 109 | 94.304 | 89.399 | 59.490 | 1.00 | 42.23 | O |
| ATOM | 3546 | O | HOH | W | 110 | 95.430 | 90.098 | 17.957 | 1.00 | 24.73 | O |
| ATOM | 3547 | O | HOH | N | 111 | 105.464 | 87.257 | 68.450 | 1.00 | 17.28 | O |
| ATOM | 3548 | O | HOH | W | 112 | 106.610 | 77.540 | 39.835 | 1.00 | 21.67 | O |
| ATOM | 3549 | O | HOH | W | 113 | 113.580 | 103.844 | 31.460 | 1.00 | 23.82 | O |
| ATOM | 3550 | O | HOH | W | 114 | 124.574 | 86.670 | 52.579 | 1.00 | 23.41 | O |
| ATOM | 3551 | O | HOH | W | 115 | 99.026 | 102.783 | 63.590 | 1.00 | 24.95 | O |
| ATOM | 3552 | O | HOH | W | 116 | 115.952 | 87.458 | 17.095 | 1.00 | 23.73 | O |
| ATOM | 3553 | O | HOH | W | 117 | 110.844 | 90.438 | 18.472 | 1.00 | 30.12 | O |
| ATOM | 3554 | O | HOH | W | 118 | 106.437 | 95.346 | 69.724 | 1.00 | 25.21 | O |
| ATOM | 3555 | O | HOH | W | 119 | 98.733 | 101.477 | 47.204 | 1.00 | 23.21 | O |
| ATOM | 3556 | O | HOH | W | 120 | 115.964 | 89.959 | 15.955 | 1.00 | 26.40 | O |
| ATOM | 3557 | O | HOH | W | 121 | 105.736 | 92.423 | 2.498 | 1.00 | 29.14 | O |
| ATOM | 3558 | O | HOH | W | 122 | 116.959 | 94.177 | 22.999 | 1.00 | 36.52 | O |
| ATOM | 3559 | O | HOH | W | 123 | 116.868 | 67.115 | 11.913 | 1.00 | 26.17 | O |
| ATOM | 3560 | O | HOH | W | 124 | 104.716 | 79.419 | 40.184 | 1.00 | 18.88 | O |
| ATOM | 3561 | O | HOH | W | 125 | 125.419 | 92.886 | 6.961 | 1.00 | 21.68 | O |
| ATOM | 3562 | O | HOH | W | 126 | 115.788 | 77.657 | 3.955 | 1.00 | 21.03 | O |
| ATOM | 3563 | O | HOH | W | 127 | 124.622 | 86.667 | 55.350 | 1.00 | 24.16 | O |
| ATOM | 3564 | O | HOH | W | 128 | 97.583 | 95.426 | 25.930 | 1.00 | 19.27 | O |
| ATOM | 3565 | O | HOH | W | 129 | 116.197 | 72.501 | 8.012 | 1.00 | 25.00 | O |
| ATOM | 3566 | O | HOH | W | 130 | 110.742 | 81.337 | 46.001 | 1.00 | 26.08 | O |
| ATOM | 3567 | O | HOH | W | 131 | 124.357 | 70.637 | 31.869 | 1.00 | 35.82 | O |
| ATOM | 3568 | O | HOH | W | 132 | 129.604 | 72.081 | 22.160 | 1.00 | 22.26 | O |
| ATOM | 3569 | O | HOH | W | 133 | 111.982 | 86.711 | 58.286 | 1.00 | 17.74 | O |
| ATOM | 3570 | O | HOH | W | 134 | 99.060 | 90.625 | 33.813 | 1.00 | 19.40 | O |
| ATOM | 3571 | O | HOH | W | 135 | 111.430 | 103.830 | 5.506 | 1.00 | 35.47 | O |
| ATOM | 3572 | O | HOH | W | 136 | 114.394 | 81.909 | 38.684 | 1.00 | 22.03 | O |
| ATOM | 3573 | O | HOH | W | 137 | 127.531 | 93.690 | 7.798 | 1.00 | 31.09 | O |
| ATOM | 3574 | O | HOH | W | 138 | 120.042 | 85.161 | −1.600 | 1.00 | 26.02 | O |
| ATOM | 3575 | O | HOW | W | 139 | 111.477 | 81.119 | 62.425 | 1.00 | 22.44 | O |
| ATOM | 3576 | O | HOH | W | 140 | 122.734 | 81.285 | 53.984 | 1.00 | 25.17 | O |
| ATOM | 3577 | O | HOH | W | 141 | 120.126 | 79.346 | 55.382 | 1.00 | 23.36 | O |
| ATOM | 3578 | O | HOH | W | 142 | 125.288 | 95.101 | 58.316 | 1.00 | 30.52 | O |
| ATOM | 3579 | O | HOH | W | 143 | 105.487 | 95.649 | 37.502 | 1.00 | 32.01 | O |
| ATOM | 3580 | O | HOH | W | 144 | 118.539 | 75.649 | 5.201 | 1.00 | 22.06 | O |
| ATOM | 3581 | O | HOH | W | 145 | 106.090 | 84.130 | 2.775 | 1.00 | 21.19 | O |
| ATOM | 3582 | O | HOH | W | 146 | 132.006 | 105.303 | 40.470 | 1.00 | 28.85 | O |
| ATOM | 3583 | O | HOH | W | 147 | 131.195 | 112.443 | 50.555 | 1.00 | 47.22 | O |
| ATOM | 3584 | O | HOH | W | 148 | 120.929 | 102.491 | 26.464 | 1.00 | 40.36 | O |
| ATOM | 3585 | O | HOH | W | 149 | 101.973 | 73.446 | 35.042 | 1.00 | 19.00 | O |
| ATOM | 3586 | O | HOH | W | 150 | 129.749 | 93.488 | 29.610 | 1.00 | 27.01 | O |
| ATOM | 3587 | O | HOH | W | 151 | 131.365 | 94.189 | 53.884 | 1.00 | 21.70 | O |
| ATOM | 3588 | O | HOH | W | 152 | 103.448 | 88.834 | 48.845 | 1.00 | 26.51 | O |
| ATOM | 3589 | O | HOH | W | 153 | 98.096 | 95.048 | 61.525 | 1.00 | 24.76 | O |
| ATOM | 3590 | O | HOH | W | 154 | 114.107 | 102.441 | 4.161 | 1.00 | 47.12 | O |
| ATOM | 3591 | O | HOH | W | 155 | 123.342 | 80.470 | 28.564 | 1.00 | 38.99 | O |
| ATOM | 3592 | O | HOH | W | 156 | 109.251 | 86.931 | 58.891 | 1.00 | 25.89 | O |
| ATOM | 3593 | O | HOH | W | 157 | 107.295 | 83.352 | 51.140 | 1.00 | 36.16 | O |
| ATOM | 3594 | O | HOH | W | 158 | 95.576 | 87.011 | 40.810 | 1.00 | 35.93 | O |
| ATOM | 3595 | O | HOH | W | 159 | 117.042 | 65.270 | 24.086 | 1.00 | 28.16 | O |
| ATOM | 3596 | O | HOH | W | 160 | 117.805 | 94.277 | 8.579 | 1.00 | 31.64 | O |
| ATOM | 3597 | O | HOH | W | 161 | 88.932 | 72.940 | 22.904 | 1.00 | 28.38 | O |
| ATOM | 3598 | O | HOH | W | 162 | 95.987 | 97.301 | 61.365 | 1.00 | 24.01 | O |
| ATOM | 3599 | O | HOH | W | 163 | 117.441 | 77.660 | 57.622 | 1.00 | 30.27 | O |
| ATOM | 3600 | O | HOH | W | 164 | 106.069 | 104.026 | 32.541 | 1.00 | 45.28 | O |
| ATOM | 3601 | O | HOH | W | 165 | 93.571 | 87.740 | 19.962 | 1.00 | 26.49 | O |
| ATOM | 3602 | O | HOH | W | 166 | 126.237 | 109.522 | 42.309 | 1.00 | 36.78 | O |
| ATOM | 3603 | O | HOH | W | 167 | 106.406 | 93.704 | 35.384 | 1.00 | 38.56 | O |
| ATOM | 3604 | O | HOH | W | 168 | 131.643 | 72.275 | 22.228 | 1.00 | 38.05 | O |
| ATOM | 3605 | O | HOH | W | 169 | 121.697 | 83.912 | 54.955 | 1.00 | 21.60 | O |
| ATOM | 3606 | O | HOH | W | 170 | 90.774 | 75.470 | 18.635 | 1.00 | 19.73 | O |
| ATOM | 3607 | O | HOH | W | 171 | 124.844 | 102.497 | 62.084 | 1.00 | 40.22 | O |
| ATOM | 3608 | O | HOH | W | 172 | 117.703 | 93.529 | 20.580 | 1.00 | 48.06 | O |
| ATOM | 3609 | O | HOH | W | 173 | 130.165 | 90.933 | 38.504 | 1.00 | 28.59 | O |
| ATOM | 3610 | O | HOH | W | 174 | 117.341 | 111.924 | 42.886 | 1.00 | 29.64 | O |
| ATOM | 3611 | O | HOH | W | 175 | 133.129 | 104.709 | 48.372 | 1.00 | 25.05 | O |
| ATOM | 3612 | O | HOH | W | 176 | 118.804 | 63.265 | 24.795 | 1.00 | 38.74 | O |
| ATOM | 3613 | O | HOH | W | 177 | 127.693 | 101.825 | 61.891 | 1.00 | 33.64 | O |
| ATOM | 3614 | O | HOH | W | 178 | 132.528 | 83.636 | 16.910 | 1.00 | 19.94 | O |
| ATOM | 3615 | O | HOH | W | 179 | 123.295 | 105.017 | 63.378 | 1.00 | 35.21 | O |
| ATOM | 3616 | O | HOH | W | 180 | 123.590 | 97.595 | 32.002 | 1.00 | 21.21 | O |
| ATOM | 3617 | O | HOH | W | 181 | 90.232 | 80.482 | 13.577 | 1.00 | 31.64 | O |
| ATOM | 3618 | O | HOH | W | 182 | 125.785 | 83.717 | 39.746 | 1.00 | 25.08 | O |
| ATOM | 3619 | O | HOH | W | 183 | 104.112 | 69.764 | 20.516 | 1.00 | 30.66 | O |
| ATOM | 3620 | O | HOH | W | 184 | 99.229 | 103.736 | 25.974 | 1.00 | 31.66 | O |
| ATOM | 3621 | O | HOH | W | 185 | 102.495 | 71.426 | 38.072 | 1.00 | 37.54 | O |
| ATOM | 3622 | O | HOH | W | 186 | 111.480 | 78.822 | 2.324 | 1.00 | 29.17 | O |
| ATOM | 3623 | O | HOH | W | 187 | 106.485 | 98.093 | 38.300 | 1.00 | 33.21 | O |
| ATOM | 3624 | O | HOH | W | 188 | 95.304 | 103.326 | 52.658 | 1.00 | 21.38 | O |
| ATOM | 3625 | O | HOH | W | 189 | 116.057 | 68.085 | 33.679 | 1.00 | 24.50 | O |
| ATOM | 3626 | O | HOH | W | 190 | 122.896 | 108.189 | 38.573 | 1.00 | 31.15 | O |
| ATOM | 3627 | O | HOH | W | 191 | 90.718 | 69.481 | 19.084 | 1.00 | 28.28 | O |
| ATOM | 3628 | O | HOH | W | 192 | 109.326 | 104.637 | 67.822 | 1.00 | 36.64 | O |
| ATOM | 3629 | O | HOH | W | 193 | 110.628 | 101.647 | 38.429 | 1.00 | 26.36 | O |
| ATOM | 3630 | O | HOH | W | 194 | 124.612 | 94.292 | 14.371 | 1.00 | 28.71 | O |
| ATOM | 3631 | O | HOH | W | 195 | 106.599 | 88.097 | 59.756 | 1.00 | 26.05 | O |
| ATOM | 3632 | O | HOH | W | 196 | 108.945 | 102.105 | 40.781 | 1.00 | 29.37 | O |
| ATOM | 3633 | O | HOH | W | 197 | 106.752 | 106.473 | 35.912 | 1.00 | 54.78 | O |
| ATOM | 3634 | O | HOH | W | 198 | 100.230 | 85.216 | 49.057 | 1.00 | 27.35 | O |
| ATOM | 3635 | O | HOH | W | 199 | 123.041 | 111.677 | 35.471 | 1.00 | 47.67 | O |
| ATOM | 3636 | O | HOH | W | 200 | 101.182 | 109.166 | 69.268 | 1.00 | 27.07 | O |
| ATOM | 3637 | O | HOH | W | 201 | 127.040 | 96.272 | 56.682 | 1.00 | 32.12 | O |
| ATOM | 3638 | O | HOH | W | 202 | 118.455 | 78.340 | 42.563 | 1.00 | 28.81 | O |
| ATOM | 3639 | O | HOH | W | 203 | 117.006 | 104.620 | 28.531 | 1.00 | 35.96 | O |
| ATOM | 3640 | O | HOH | W | 204 | 116.887 | 94.957 | 30.413 | 1.00 | 34.56 | O |
| ATOM | 3641 | O | HOH | W | 205 | 114.906 | 70.745 | 38.259 | 1.00 | 23.34 | O |
| ATOM | 3642 | O | HOH | W | 206 | 122.202 | 96.362 | 1.112 | 1.00 | 43.16 | O |
| ATOM | 3643 | O | HOH | W | 207 | 103.300 | 70.307 | 41.014 | 1.00 | 30.46 | O |
| ATOM | 3644 | O | HOH | W | 208 | 117.579 | 77.374 | 1.437 | 1.00 | 39.25 | O |
| ATOM | 3645 | O | HOH | W | 209 | 114.149 | 91.956 | 34.139 | 1.00 | 29.03 | O |
| ATOM | 3646 | O | HOH | W | 210 | 119.637 | 66.971 | 10.956 | 1.00 | 32.38 | O |
| ATOM | 3647 | O | HOH | W | 211 | 100.914 | 110.722 | 53.327 | 1.00 | 31.33 | O |
| ATOM | 3648 | O | HOH | W | 212 | 111.079 | 108.871 | 63.668 | 1.00 | 40.95 | O |
| ATOM | 3649 | O | HOH | W | 213 | 111.662 | 105.911 | 67.214 | 1.00 | 29.55 | O |
| ATOM | 3650 | O | HOH | W | 214 | 117.706 | 81.463 | 62.184 | 1.00 | 28.79 | O |
| ATOM | 3651 | O | HOH | W | 215 | 112.977 | 80.319 | 46.199 | 1.00 | 31.02 | O |
| ATOM | 3652 | O | HOH | W | 216 | 122.205 | 92.948 | 4.192 | 1.00 | 37.99 | O |
| ATOM | 3653 | O | HOH | W | 217 | 90.574 | 83.616 | 25.345 | 1.00 | 24.10 | O |
| ATOM | 3654 | O | HOH | W | 218 | 122.706 | 87.159 | 63.652 | 1.00 | 41.77 | O |
| ATOM | 3655 | O | HOH | W | 219 | 121.421 | 77.309 | 47.966 | 1.00 | 29.53 | O |
| ATOM | 3656 | O | HOH | W | 220 | 116.793 | 82.581 | 39.116 | 1.00 | 21.60 | O |
| ATOM | 3657 | O | HOH | W | 221 | 101.152 | 84.895 | 41.351 | 1.00 | 35.44 | O |
| ATOM | 3658 | O | HOH | W | 222 | 103.796 | 96.354 | 69.460 | 1.00 | 29.96 | O |
| ATOM | 3659 | O | HOH | W | 223 | 125.911 | 84.974 | 37.549 | 1.00 | 31.04 | O |
| ATOM | 3660 | O | HOH | W | 224 | 115.290 | 111.114 | 46.966 | 1.00 | 27.38 | O |
| ATOM | 3661 | O | HOH | W | 225 | 99.140 | 91.702 | 64.223 | 1.00 | 25.74 | O |
| ATOM | 3662 | O | HOH | W | 226 | 114.620 | 105.119 | 71.256 | 1.00 | 37.74 | O |
| ATOM | 3663 | O | HOH | W | 227 | 89.198 | 84.691 | 12.406 | 1.00 | 29.75 | O |
| ATOM | 3664 | O | HOH | W | 228 | 115.763 | 76.050 | 39.822 | 1.00 | 27.28 | O |
| ATOM | 3665 | O | HOH | W | 229 | 92.305 | 78.313 | 34.056 | 1.00 | 36.00 | O |
| ATOM | 3666 | O | HOH | W | 230 | 93.755 | 89.711 | 21.852 | 1.00 | 42.00 | O |
| ATOM | 3667 | O | HOH | W | 231 | 97.093 | 107.724 | 53.833 | 1.00 | 32.19 | O |
| ATOM | 3668 | O | HOH | W | 232 | 122.139 | 84.813 | 52.087 | 1.00 | 26.81 | O |
| ATOM | 3669 | O | HOH | W | 233 | 129.655 | 86.783 | 30.773 | 1.00 | 33.02 | O |
| ATOM | 3670 | O | HOH | W | 234 | 123.909 | 116.098 | 54.301 | 1.00 | 28.40 | O |
| ATOM | 3671 | O | HOH | W | 235 | 108.987 | 94.563 | 14.792 | 1.00 | 36.10 | O |
| ATOM | 3672 | O | HOH | W | 236 | 95.725 | 113.425 | 47.459 | 1.00 | 38.40 | O |
| ATOM | 3673 | O | HOH | W | 237 | 109.794 | 94.573 | 23.380 | 1.00 | 38.30 | O |
| ATOM | 3674 | O | HOH | W | 238 | 105.801 | 113.664 | 49.721 | 1.00 | 23.96 | O |
| ATOM | 3675 | O | HOH | W | 239 | 90.054 | 82.779 | 10.757 | 1.00 | 42.50 | O |
| ATOM | 3676 | O | HOH | W | 240 | 109.364 | 61.210 | 7.747 | 1.00 | 36.87 | O |
| ATOM | 3677 | O | HOH | W | 241 | 125.867 | 105.241 | 62.792 | 1.00 | 40.64 | O |
| ATOM | 3678 | O | HOH | W | 242 | 98.468 | 94.340 | 64.096 | 1.00 | 32.57 | O |
| ATOM | 3679 | O | HOH | W | 243 | 121.840 | 103.159 | 64.633 | 1.00 | 34.33 | O |
| ATOM | 3680 | O | HOH | W | 244 | 126.079 | 67.126 | 14.188 | 1.00 | 30.65 | O |
| ATOM | 3681 | O | HOH | W | 245 | 96.658 | 87.655 | 58.860 | 1.00 | 40.59 | O |
| ATOM | 3682 | O | HOH | W | 246 | 127.962 | 88.119 | 50.340 | 1.00 | 29.71 | O |
| ATOM | 3683 | O | HOH | W | 247 | 110.338 | 73.497 | 42.611 | 1.00 | 38.35 | O |
| ATOM | 3684 | O | HOH | W | 248 | 114.772 | 97.055 | 69.663 | 1.00 | 36.50 | O |
| ATOM | 3685 | O | HOH | W | 249 | 108.869 | 101.284 | 28.764 | 1.00 | 37.52 | O |
| ATOM | 3686 | O | HOH | W | 250 | 107.224 | 107.639 | 38.231 | 1.00 | 36.41 | O |
| ATOM | 3687 | O | HOH | W | 251 | 96.390 | 93.165 | 6.888 | 1.00 | 35.89 | O |
| ATOM | 3688 | O | HOH | W | 252 | 110.141 | 77.524 | 0.949 | 1.00 | 37.62 | O |
| ATOM | 3689 | O | HOH | W | 253 | 111.788 | 93.641 | 21.577 | 1.00 | 33.63 | O |
| ATOM | 3690 | O | HOH | W | 254 | 92.752 | 82.228 | 10.382 | 1.00 | 26.30 | O |
| ATOM | 3691 | O | HOH | W | 255 | 108.583 | 102.139 | 34.491 | 1.00 | 47.03 | O |
| ATOM | 3692 | O | HOH | W | 256 | 94.018 | 92.160 | 18.054 | 1.00 | 43.36 | O |
| ATOM | 3693 | O | HOH | W | 257 | 135.205 | 93.985 | 47.235 | 1.00 | 41.66 | O |
| ATOM | 3694 | O | HOH | W | 258 | 127.096 | 114.238 | 52.111 | 1.00 | 34.50 | O |
| ATOM | 3695 | O | HOH | W | 259 | 114.324 | 67.450 | 36.606 | 1.00 | 44.07 | O |
| ATOM | 3696 | O | HOH | W | 260 | 94.360 | 82.674 | 7.084 | 1.00 | 36.34 | O |
| ATOM | 3697 | O | HOH | W | 261 | 127.924 | 81.806 | 34.620 | 1.00 | 47.40 | O |
| ATOM | 3698 | O | HOH | W | 262 | 128.992 | 111.561 | 52.471 | 1.00 | 38.42 | O |
| ATOM | 3699 | O | HOH | W | 263 | 119.300 | 108.625 | 62.911 | 1.00 | 27.22 | O |
| ATOM | 3700 | O | HOH | W | 264 | 130.357 | 89.245 | 50.746 | 1.00 | 34.49 | O |
| ATOM | 3701 | O | HOH | W | 265 | 118.959 | 94.646 | 26.220 | 1.00 | 26.18 | O |
| ATOM | 3702 | O | HOH | W | 266 | 96.163 | 91.058 | 49.536 | 1.00 | 40.12 | O |
| ATOM | 3703 | O | HOH | W | 267 | 106.631 | 105.802 | 66.726 | 1.00 | 31.18 | O |
| ATOM | 3704 | O | HOH | W | 268 | 95.271 | 106.317 | 53.036 | 1.00 | 33.98 | O |
| ATOM | 3705 | O | HOH | W | 269 | 118.054 | 119.134 | 49.885 | 1.00 | 31.94 | O |
| ATOM | 3706 | O | HOH | W | 270 | 127.717 | 106.148 | 61.501 | 1.00 | 45.35 | O |
| ATOM | 3707 | O | HOH | W | 271 | 106.742 | 86.817 | 57.315 | 1.00 | 33.97 | O |
| ATOM | 3708 | O | HOH | W | 272 | 101.720 | 96.187 | 40.313 | 1.00 | 49.19 | O |
| ATOM | 3709 | O | HOH | W | 273 | 104.310 | 75.155 | 38.637 | 1.00 | 33.77 | O |
| ATOM | 3710 | O | HOH | W | 274 | 118.372 | 66.655 | 34.338 | 1.00 | 42.60 | O |
| ATOM | 3711 | O | HOH | W | 275 | 116.363 | 78.614 | 40.461 | 1.00 | 25.63 | O |
| ATOM | 3712 | O | HOH | W | 276 | 102.000 | 84.493 | 60.043 | 1.00 | 49.15 | O |
| ATOM | 3713 | O | HOH | W | 277 | 104.014 | 98.282 | 40.327 | 1.00 | 42.59 | O |
| ATOM | 3714 | O | HOH | W | 278 | 113.856 | 90.499 | 67.957 | 1.00 | 31.25 | O |
| ATOM | 3715 | O | HOH | W | 279 | 123.554 | 75.061 | 32.158 | 1.00 | 32.79 | O |
| ATOM | 3716 | O | HOH | W | 280 | 130.061 | 87.059 | 44.812 | 1.00 | 41.79 | O |
| ATOM | 3717 | O | HOH | W | 281 | 132.479 | 104.983 | 33.707 | 1.00 | 47.17 | O |
| ATOM | 3718 | O | HOH | W | 282 | 108.381 | 85.000 | 56.035 | 1.00 | 26.57 | O |
| ATOM | 3719 | O | HOH | W | 283 | 128.718 | 100.225 | 32.013 | 1.00 | 33.37 | O |
| ATOM | 3720 | O | HOH | W | 284 | 106.815 | 72.080 | 7.692 | 1.00 | 31.60 | O |
| ATOM | 3721 | O | HOH | W | 285 | 107.264 | 96.265 | 22.453 | 1.00 | 46.65 | O |
| ATOM | 3722 | O | HOH | W | 286 | 127.791 | 88.594 | 31.966 | 1.00 | 33.75 | O |
| ATOM | 3723 | O | HOH | W | 287 | 103.566 | 71.677 | 8.060 | 1.00 | 30.69 | O |
| ATOM | 3724 | O | HOH | W | 288 | 110.616 | 111.068 | 64.708 | 1.00 | 43.04 | O |
| ATOM | 3725 | O | HOH | W | 289 | 119.879 | 65.440 | 15.204 | 1.00 | 34.67 | O |
| ATOM | 3726 | O | HOH | W | 290 | 122.854 | 72.937 | 21.033 | 1.00 | 23.84 | O |
| ATOM | 3727 | O | HOH | W | 291 | 108.017 | 74.762 | 41.281 | 1.00 | 44.30 | O |
| ATOM | 3728 | O | HOH | W | 292 | 107.705 | 103.629 | 30.523 | 1.00 | 49.92 | O |
| ATOM | 3729 | O | HOH | W | 293 | 101.801 | 110.605 | 51.082 | 1.00 | 35.59 | O |
| ATOM | 3730 | O | HOH | W | 294 | 107.885 | 102.093 | 26.076 | 1.00 | 35.88 | O |
| ATOM | 3731 | O | HOH | W | 295 | 103.729 | 77.333 | 2.620 | 1.00 | 30.24 | O |
| ATOM | 3732 | O | HOH | W | 296 | 121.252 | 79.872 | 39.465 | 1.00 | 29.98 | O |
| ATOM | 3733 | O | HOH | W | 297 | 131.395 | 74.452 | 24.405 | 1.00 | 50.96 | O |
| ATOM | 3734 | O | HOH | W | 298 | 108.904 | 93.004 | 34.923 | 1.00 | 38.69 | O |
| ATOM | 3735 | O | HOH | W | 299 | 123.295 | 81.534 | 37.351 | 1.00 | 33.13 | O |
| ATOM | 3736 | O | HOH | W | 300 | 124.590 | 90.880 | 62.781 | 1.00 | 42.97 | O |
| ATOM | 3737 | O | HOH | W | 301 | 94.929 | 72.229 | 30.511 | 1.00 | 24.48 | O |
| ATOM | 3738 | O | HOH | W | 302 | 128.429 | 84.318 | 42.172 | 1.00 | 38.09 | O |
| ATOM | 3739 | O | HOH | W | 303 | 121.859 | 111.580 | 43.480 | 1.00 | 25.71 | O |
| ATOM | 3740 | O | HOH | W | 304 | 133.428 | 89.012 | 15.848 | 1.00 | 36.07 | O |
| ATOM | 3741 | O | HOH | W | 305 | 118.107 | 115.897 | 61.799 | 1.00 | 44.42 | O |
| ATOM | 3742 | O | HOH | W | 306 | 120.786 | 83.869 | 61.330 | 1.00 | 41.08 | O |
| ATOM | 3743 | O | HOH | W | 307 | 92.823 | 94.296 | 28.950 | 1.00 | 36.81 | O |
| ATOM | 3744 | O | HOH | W | 308 | 105.369 | 100.860 | 66.652 | 1.00 | 43.43 | O |
| ATOM | 3745 | O | HOH | W | 309 | 130.121 | 88.025 | 23.805 | 1.00 | 32.52 | O |
| ATOM | 3746 | O | HOH | W | 310 | 134.200 | 101.165 | 42.762 | 1.00 | 38.44 | O |
| ATOM | 3747 | O | HOH | W | 311 | 110.481 | 107.822 | 42.814 | 1.00 | 41.85 | O |
| ATOM | 3748 | O | HOH | W | 312 | 120.646 | 95.823 | 65.405 | 1.00 | 34.54 | O |
| ATOM | 3749 | O | HOH | W | 313 | 106.862 | 84.472 | 53.518 | 1.00 | 41.79 | O |
| ATOM | 3750 | O | HOH | W | 314 | 129.915 | 88.108 | 26.255 | 1.00 | 38.33 | O |
| ATOM | 3751 | O | HOH | W | 315 | 96.353 | 117.845 | 46.682 | 1.00 | 39.22 | O |
| ATOM | 3752 | O | HOH | W | 316 | 99.674 | 94.586 | 54.234 | 1.00 | 23.64 | O |
| ATOM | 3753 | O | HOH | W | 317 | 101.607 | 75.493 | 38.508 | 1.00 | 37.24 | O |
| ATOM | 3754 | O | HOH | W | 318 | 95.646 | 75.136 | 4.410 | 1.00 | 40.62 | O |
| ATOM | 3755 | O | HOH | W | 319 | 91.281 | 68.335 | 23.622 | 1.00 | 28.54 | O |
| ATOM | 3756 | O | HOH | W | 320 | 105.510 | 113.836 | 58.686 | 1.00 | 39.09 | O |
| ATOM | 3757 | O | HOH | W | 321 | 96.119 | 78.195 | 36.019 | 1.00 | 34.94 | O |
| ATOM | 3758 | O | HOH | W | 322 | 131.260 | 108.207 | 41.625 | 1.00 | 42.53 | O |
| ATOM | 3759 | O | HOH | W | 323 | 113.734 | 110.963 | 42.508 | 1.00 | 33.59 | O |
| ATOM | 3760 | O | HOH | W | 324 | 126.412 | 70.628 | 7.827 | 1.00 | 30.84 | O |
| ATOM | 3761 | O | HOH | W | 325 | 119.275 | 62.088 | 22.394 | 1.00 | 46.78 | O |
| ATOM | 3762 | O | HOH | W | 326 | 137.211 | 95.559 | 48.509 | 1.00 | 30.78 | O |
| ATOM | 3763 | O | HOH | W | 327 | 138.842 | 97.857 | 50.465 | 1.00 | 44.11 | O |
| ATOM | 3764 | O | HOH | W | 328 | 103.515 | 70.067 | 15.680 | 1.00 | 38.12 | O |
| ATOM | 3765 | O | HOH | W | 329 | 117.214 | 68.108 | 6.574 | 1.00 | 40.33 | O |
| ATOM | 3766 | O | HOH | W | 330 | 97.619 | 69.258 | 5.792 | 1.00 | 50.34 | O |
| ATOM | 3767 | O | HOH | W | 331 | 130.617 | 97.384 | 32.045 | 1.00 | 29.75 | O |
| ATOM | 3768 | O | HOH | W | 332 | 113.346 | 74.677 | 2.563 | 1.00 | 31.10 | O |
| ATOM | 3769 | O | HOH | W | 333 | 115.466 | 93.421 | 7.713 | 1.00 | 38.98 | O |
| ATOM | 3770 | O | HOH | W | 334 | 99.078 | 71.097 | 9.233 | 1.00 | 45.81 | O |
| ATOM | 3771 | O | HOH | W | 335 | 129.424 | 93.761 | 3.813 | 1.00 | 40.30 | O |
| ATOM | 3772 | O | HOH | W | 336 | 120.981 | 74.330 | 36.001 | 1.00 | 44.95 | O |
| ATOM | 3773 | O | HOH | W | 337 | 123.358 | 80.725 | 39.502 | 1.00 | 40.54 | O |
| ATOM | 3774 | O | HOH | W | 338 | 110.424 | 66.848 | 13.059 | 1.00 | 29.06 | O |
| ATOM | 3775 | O | HOH | W | 339 | 114.322 | 97.973 | 25.770 | 1.00 | 40.40 | O |
| ATOM | 3776 | O | HOH | W | 340 | 123.331 | 86.735 | 15.530 | 1.00 | 34.68 | O |
| ATOM | 3777 | O | HOH | W | 341 | 104.964 | 96.819 | 34.480 | 1.00 | 41.52 | O |
| ATOM | 3778 | O | HOH | W | 342 | 116.282 | 92.182 | 4.948 | 1.00 | 48.87 | O |
| ATOM | 3779 | O | HOH | W | 343 | 113.171 | 94.504 | 1.763 | 1.00 | 40.20 | O |
| ATOM | 3780 | O | HOH | W | 344 | 110.679 | 78.202 | 44.920 | 1.00 | 29.15 | O |
| ATOM | 3781 | O | HOH | W | 345 | 96.899 | 92.602 | 34.046 | 1.00 | 36.09 | O |
| ATOM | 3782 | O | HOH | W | 346 | 97.017 | 94.031 | 44.509 | 1.00 | 35.09 | O |
| ATOM | 3783 | O | HOH | W | 347 | 127.343 | 80.853 | 41.914 | 1.00 | 40.19 | O |
| ATOM | 3784 | O | HOH | W | 348 | 129.403 | 73.026 | 27.482 | 1.00 | 49.52 | O |
| ATOM | 3785 | O | HOH | W | 349 | 97.294 | 99.927 | 67.290 | 1.00 | 49.09 | O |
| ATOM | 3786 | O | HOH | W | 350 | 103.094 | 70.478 | 11.537 | 1.00 | 27.82 | O |
| ATOM | 3787 | O | HOH | W | 351 | 135.618 | 99.151 | 42.987 | 1.00 | 32.34 | O |
| ATOM | 3788 | O | HOH | W | 352 | 93.987 | 98.210 | 22.281 | 1.00 | 43.19 | O |
| ATOM | 3789 | O | HOH | W | 353 | 95.003 | 89.098 | 56.539 | 1.00 | 41.16 | O |
| ATOM | 3790 | O | HOH | W | 354 | 113.529 | 64.013 | 19.053 | 1.00 | 30.38 | O |
| ATOM | 3791 | O | HOH | W | 355 | 124.457 | 72.406 | 4.180 | 1.00 | 19.05 | O |
| ATOM | 3792 | O | HOH | W | 356 | 104.962 | 97.098 | 4.965 | 1.00 | 38.23 | O |
| ATOM | 3793 | O | HOH | W | 357 | 117.836 | 81.953 | −3.768 | 1.00 | 34.88 | O |
| ATOM | 3794 | O | HOH | W | 358 | 95.824 | 91.230 | 53.978 | 1.00 | 35.65 | O |
| ATOM | 3795 | O | HOH | W | 359 | 127.554 | 103.710 | 30.349 | 1.00 | 43.90 | O |
| ATOM | 3796 | O | HOH | W | 360 | 125.147 | 99.275 | 60.105 | 1.00 | 33.00 | O |
| ATOM | 3797 | O | HOH | W | 361 | 95.829 | 93.859 | 53.829 | 1.00 | 51.24 | O |
| ATOM | 3798 | O | HOH | W | 362 | 98.062 | 79.975 | 5.765 | 1.00 | 29.37 | O |
| ATOM | 3799 | O | HOH | W | 363 | 97.884 | 102.272 | 66.221 | 1.00 | 45.40 | O |
| ATOM | 3800 | O | HOH | W | 364 | 103.575 | 92.945 | 6.473 | 1.00 | 31.11 | O |
| ATOM | 3801 | O | HOH | W | 365 | 107.873 | 110.644 | 66.583 | 1.00 | 43.17 | O |
| ATOM | 3802 | O | HOH | W | 366 | 109.412 | 109.207 | 68.363 | 0.50 | 36.17 | O |
| ATOM | 3803 | O | HOH | W | 367 | 121.077 | 106.718 | 34.149 | 1.00 | 35.29 | O |
| ATOM | 3804 | O | HOH | W | 368 | 126.056 | 84.065 | 50.738 | 1.00 | 48.74 | O |
| ATOM | 3805 | O | HOH | W | 369 | 108.800 | 109.497 | 43.972 | 1.00 | 53.52 | O |
| ATOM | 3806 | O | HOH | W | 370 | 122.969 | 85.340 | 59.208 | 1.00 | 38.67 | O |
| ATOM | 3807 | O | HOH | W | 373 | 108.464 | 97.322 | 69.773 | 1.00 | 30.74 | O |
| ATOM | 3808 | O | HOH | W | 372 | 121.114 | 96.932 | 67.946 | 1.00 | 36.89 | O |
| ATOM | 3809 | O | HOH | W | 373 | 129.671 | 82.063 | 25.469 | 1.00 | 27.70 | O |
| ATOM | 3810 | O | HOH | W | 374 | 114.108 | 108.161 | 37.457 | 1.00 | 39.08 | O |
| ATOM | 3811 | O | HOH | W | 375 | 136.149 | 105.331 | 54.491 | 1.00 | 60.33 | O |
| ATOM | 3812 | O | HOH | W | 376 | 133.231 | 102.907 | 41.106 | 1.00 | 53.33 | O |
| ATOM | 3813 | O | HOH | W | 377 | 106.995 | 84.626 | 48.633 | 1.00 | 39.72 | O |
| ATOM | 3814 | O | HOH | W | 378 | 90.229 | 71.012 | 29.341 | 1.00 | 34.43 | O |
| ATOM | 3815 | O | HOH | W | 379 | 103.884 | 109.645 | 43.266 | 1.00 | 42.49 | O |
| ATOM | 3816 | O | HOH | W | 380 | 86.222 | 84.556 | 12.175 | 1.00 | 37.26 | O |
| ATOM | 3817 | O | HOH | W | 381 | 110.735 | 66.256 | 37.531 | 1.00 | 27.72 | O |
| ATOM | 3818 | O | HOH | W | 382 | 101.375 | 68.664 | 15.543 | 1.00 | 38.16 | O |
| ATOM | 3819 | O | HOH | W | 383 | 101.379 | 80.205 | 42.477 | 1.00 | 49.16 | O |
| ATOM | 3820 | O | HOH | W | 384 | 115.074 | 112.258 | 44.635 | 1.00 | 36.81 | O |
| ATOM | 3821 | O | HOH | W | 385 | 136.960 | 96.344 | 43.738 | 1.00 | 34.94 | O |
| ATOM | 3822 | O | HOH | W | 386 | 108.817 | 85.628 | 2.366 | 1.00 | 29.82 | O |
| ATOM | 3823 | O | HOH | W | 387 | 110.834 | 104.161 | 37.862 | 1.00 | 43.16 | O |
| ATOM | 3824 | O | HOH | W | 388 | 109.798 | 93.518 | 19.112 | 1.00 | 40.29 | O |
| ATOM | 3825 | O | HOH | W | 389 | 127.841 | 86.030 | 15.581 | 1.00 | 47.18 | O |
| ATOM | 3826 | O | HOH | W | 390 | 101.489 | 78.445 | 41.185 | 1.00 | 39.44 | O |
| ATOM | 3827 | O | HOH | W | 391 | 125.979 | 82.516 | 32.137 | 1.00 | 38.94 | O |
| ATOM | 3828 | O | HOH | W | 392 | 119.793 | 88.095 | −1.891 | 1.00 | 39.85 | O |
| ATOM | 3829 | O | HOH | W | 393 | 114.445 | 115.370 | 44.056 | 1.00 | 46.69 | O |
| ATOM | 3830 | O | HOH | W | 394 | 121.139 | 81.211 | 61.458 | 1.00 | 49.19 | O |
| ATOM | 3831 | O | HOH | W | 395 | 99.178 | 81.067 | 41.904 | 1.00 | 57.83 | O |
| ATOM | 3832 | O | HOH | W | 396 | 110.246 | 83.926 | 0.951 | 1.00 | 37.51 | O |
| ATOM | 3833 | O | HOH | W | 397 | 119.704 | 93.595 | 15.834 | 1.00 | 39.85 | O |
| ATOM | 3834 | O | HOH | W | 398 | 117.641 | 75.200 | 53.911 | 1.00 | 42.12 | O |
| ATOM | 3835 | O | HOH | W | 399 | 107.980 | 79.490 | −1.122 | 1.00 | 34.35 | O |
| ATOM | 3836 | O | HOH | W | 400 | 89.045 | 80.331 | 22.955 | 1.00 | 36.28 | O |
| ATOM | 3837 | O | HOH | W | 401 | 100.822 | 79.582 | 0.491 | 1.00 | 53.22 | O |
| ATOM | 3838 | O | HOH | W | 402 | 121.500 | 107.842 | 36.505 | 1.00 | 33.41 | O |
| ATOM | 3839 | O | HOH | W | 403 | 122.768 | 99.004 | 74.869 | 1.00 | 37.82 | O |
| ATOM | 3840 | O | HOH | W | 404 | 126.818 | 86.695 | 1.352 | 1.00 | 38.16 | O |
| ATOM | 3841 | O | HOH | W | 405 | 126.980 | 99.677 | 68.733 | 1.00 | 44.14 | O |
| ATOM | 3842 | O | HOH | W | 406 | 115.703 | 62.672 | 22.437 | 1.00 | 38.33 | O |
| ATOM | 3843 | O | HOH | W | 407 | 85.280 | 86.114 | 14.392 | 1.00 | 43.80 | O |
| ATOM | 3844 | O | HOH | W | 408 | 93.731 | 93.599 | 62.752 | 1.00 | 36.63 | O |
| ATOM | 3845 | O | HOH | W | 409 | 94.185 | 72.315 | 13.049 | 1.00 | 32.12 | O |
| ATOM | 3846 | O | HOH | W | 410 | 114.924 | 93.757 | 67.159 | 1.00 | 38.60 | O |
| ATOM | 3847 | O | HOH | W | 411 | 123.665 | 99.722 | 77.097 | 1.00 | 38.57 | O |
| ATOM | 3848 | O | HOH | W | 412 | 97.484 | 71.219 | 14.834 | 1.00 | 31.18 | O |
| ATOM | 3849 | O | HOH | W | 413 | 131.013 | 120.163 | 45.927 | 1.00 | 48.50 | O |
| ATOM | 3850 | O | HOH | W | 414 | 95.976 | 92.286 | 47.441 | 1.00 | 51.96 | O |
| ATOM | 3851 | O | HOH | W | 415 | 122.799 | 80.921 | 58.368 | 1.00 | 44.17 | O |
| ATOM | 3852 | O | HOH | W | 416 | 120.917 | 116.269 | 60.833 | 1.00 | 43.28 | O |
| ATOM | 3853 | O | HOH | W | 417 | 118.678 | 112.050 | 62.936 | 1.00 | 38.11 | O |
| ATOM | 3854 | O | HOH | W | 418 | 123.810 | 90.539 | −1.964 | 1.00 | 45.13 | O |
| ATOM | 3855 | O | HOH | W | 419 | 104.054 | 113.992 | 47.803 | 1.00 | 41.51 | O |
| ATOM | 3856 | O | HOH | W | 420 | 117.762 | 90.295 | 1.454 | 1.00 | 40.56 | O |
| ATOM | 3857 | O | HOH | W | 421 | 119.046 | 90.492 | 3.324 | 1.00 | 33.27 | O |
| ATOM | 3858 | O | HOH | W | 422 | 95.910 | 108.557 | 49.319 | 1.00 | 37.44 | O |
| ATOM | 3859 | O | HOH | W | 423 | 107.686 | 71.888 | 28.074 | 1.00 | 17.75 | O |
| ATOM | 3860 | O | HOH | W | 424 | 92.935 | 80.164 | 12.604 | 1.00 | 36.91 | O |
| ATOM | 3861 | O | HOH | W | 425 | 129.548 | 86.199 | 47.846 | 1.00 | 46.29 | O |
| ATOM | 3862 | O | HOH | W | 426 | 124.369 | 114.506 | 42.395 | 1.00 | 36.49 | O |
| ATOM | 3863 | O | HOH | W | 427 | 115.776 | 97.490 | 28.708 | 1.00 | 45.34 | O |
| ATOM | 3864 | O | HOH | W | 428 | 131.668 | 86.070 | 17.163 | 1.00 | 40.11 | O |
| ATOM | 3865 | O | HOH | W | 429 | 105.471 | 103.488 | 67.483 | 1.00 | 34.36 | O |
| ATOM | 3866 | O | HOH | W | 430 | 117.229 | 116.018 | 45.785 | 1.00 | 50.05 | O |
| ATOM | 3867 | O | HOH | W | 431 | 126.235 | 113.179 | 41.446 | 1.00 | 46.09 | O |
| ATOM | 3868 | O | HOH | W | 432 | 91.325 | 82.301 | 28.347 | 1.00 | 34.78 | O |
| ATOM | 3869 | O | HOH | W | 433 | 101.259 | 112.151 | 55.333 | 1.00 | 42.05 | O |
| ATOM | 3870 | O | HOH | W | 434 | 113.000 | 109.506 | 38.997 | 1.00 | 43.41 | O |
| ATOM | 3871 | O | HOH | W | 435 | 110.650 | 85.829 | 56.398 | 1.00 | 42.06 | O |
| ATOM | 3872 | O | HOH | W | 436 | 95.246 | 83.685 | 38.392 | 1.00 | 44.60 | O |
| ATOM | 3873 | O | HOH | W | 437 | 101.143 | 82.917 | 47.755 | 1.00 | 33.97 | O |
| ATOM | 3874 | O | HOH | W | 438 | 121.295 | 78.002 | 33.489 | 1.00 | 34.74 | O |
| ATOM | 3875 | O | HOH | W | 439 | 88.779 | 84.917 | 3.593 | 1.00 | 39.00 | O |
| ATOM | 3876 | O | HOH | W | 440 | 131.080 | 106.220 | 35.506 | 1.00 | 33.99 | O |
| ATOM | 3877 | O | HOH | W | 441 | 134.850 | 91.977 | 49.867 | 1.00 | 38.43 | O |
| ATOM | 3878 | O | HOH | W | 442 | 123.043 | 121.175 | 51.067 | 1.00 | 43.09 | O |
| ATOM | 3879 | O | HOH | W | 443 | 127.035 | 87.295 | 55.782 | 1.00 | 42.98 | O |
| ATOM | 3880 | O | HOH | W | 444 | 111.809 | 77.877 | 41.608 | 1.00 | 35.39 | O |
| ATOM | 3881 | O | HOH | W | 445 | 132.947 | 92.230 | 52.593 | 1.00 | 41.36 | O |
| ATOM | 3882 | O | HOH | W | 446 | 122.448 | 113.219 | 58.074 | 1.00 | 43.40 | O |
| ATOM | 3883 | O | HOH | W | 447 | 92.920 | 89.483 | 15.423 | 1.00 | 44.60 | O |
| ATOM | 3884 | O | HOH | W | 448 | 105.707 | 69.305 | 18.849 | 1.00 | 42.00 | O |
| ATOM | 3885 | O | HOH | W | 449 | 115.732 | 74.825 | 2.079 | 1.00 | 38.15 | O |
| ATOM | 3886 | O | HOH | W | 450 | 106.783 | 69.295 | 16.634 | 1.00 | 38.35 | O |
| ATOM | 3887 | O | HOH | W | 451 | 94.584 | 87.266 | 44.612 | 1.00 | 37.75 | O |
| ATOM | 3888 | O | HOH | W | 452 | 114.497 | 78.410 | 59.139 | 1.00 | 42.30 | O |
| ATOM | 3889 | O | HOH | W | 453 | 94.990 | 88.463 | 62.371 | 1.00 | 40.54 | O |
| ATOM | 3890 | O | HOH | W | 454 | 121.889 | 93.823 | 13.425 | 1.00 | 22.82 | O |
| ATOM | 3891 | O | HOH | W | 455 | 114.929 | 96.736 | 30.735 | 1.00 | 32.96 | O |
| ATOM | 3892 | O | HOH | W | 456 | 112.288 | 103.726 | 28.694 | 1.00 | 41.61 | O |
| ATOM | 3893 | O | HOH | W | 457 | 125.085 | 100.119 | 73.157 | 1.00 | 45.76 | O |
| ATOM | 3894 | O | HOH | W | 458 | 130.763 | 85.024 | 7.018 | 1.00 | 11.45 | O |
| ATOM | 3895 | O | HOH | W | 459 | 124.052 | 107.273 | 54.264 | 1.00 | 17.21 | O |
| ATOM | 3896 | O | HOH | W | 460 | 107.667 | 70.406 | 20.379 | 1.00 | 15.93 | O |
| ATOM | 3897 | O | HOH | W | 461 | 104.452 | 80.837 | 1.427 | 1.00 | 26.18 | O |
| ATOM | 3898 | O | HOH | W | 462 | 108.682 | 104.943 | 64.735 | 1.00 | 21.79 | O |
| ATOM | 3899 | O | HOH | W | 463 | 115.691 | 114.305 | 61.372 | 1.00 | 31.42 | O |
| ATOM | 3900 | O | HOH | W | 464 | 126.407 | 80.687 | 3.057 | 1.00 | 33.95 | O |
| ATOM | 3901 | O | HOH | W | 465 | 122.229 | 79.934 | 33.531 | 1.00 | 38.82 | O |
| ATOM | 3902 | O | HOH | W | 466 | 124.736 | 83.349 | 1.592 | 1.00 | 33.53 | O |
| ATOM | 3903 | O | HOH | W | 467 | 114.267 | 98.847 | 66.692 | 1.00 | 33.77 | O |
| ATOM | 3904 | O | HOH | W | 468 | 130.037 | 86.270 | 41.427 | 1.00 | 37.20 | O |
| ATOM | 3905 | O | HOH | W | 469 | 104.287 | 85.763 | 59.247 | 1.00 | 33.08 | O |
| ATOM | 3906 | O | HOH | W | 470 | 113.444 | 88.328 | −2.313 | 1.00 | 30.83 | O |
| ATOM | 3907 | O | HOH | W | 471 | 105.236 | 85.072 | 62.709 | 1.00 | 35.13 | O |
| ATOM | 3908 | O | HOH | W | 472 | 114.514 | 70.605 | 8.718 | 1.00 | 32.96 | O |
| ATOM | 3909 | O | HOH | W | 473 | 108.573 | 108.554 | 34.410 | 0.50 | 43.00 | O |
| ATOM | 3910 | O | HOH | W | 474 | 96.075 | 119.134 | 52.779 | 1.00 | 32.24 | O |
| ATOM | 3911 | O | HOH | W | 475 | 96.858 | 110.022 | 55.144 | 1.00 | 35.37 | O |
| ATOM | 3912 | O | HOH | W | 476 | 115.134 | 67.278 | 3.391 | 1.00 | 41.66 | O |
| ATOM | 3913 | O | HOH | W | 477 | 124.446 | 73.371 | 29.788 | 1.00 | 44.39 | O |
| ATOM | 3914 | O | HOH | W | 478 | 126.924 | 93.200 | 2.834 | 1.00 | 33.53 | O |
| ATOM | 3915 | O | HOH | W | 479 | 112.338 | 97.570 | 8.004 | 1.00 | 33.46 | O |
| ATOM | 3916 | O | HOH | W | 480 | 134.955 | 96.907 | 39.439 | 1.00 | 37.10 | O |
| ATOM | 3917 | O | HOH | W | 481 | 112.244 | 77.347 | 54.049 | 1.00 | 37.73 | O |

TABLE 2
Coordinates of human neutrophil elastase in complex with inhibitor
of formula I in space group P6₃

| | | | | | | | | | | | |
|---|---|---|---|---|---|---|---|---|---|---|---|
| ATOM | 1 | N | ILE | A | 16 | 40.574 | −7.461 | −9.324 | 1.00 | 11.24 | N |
| ATOM | 2 | CA | ILE | A | 16 | 39.258 | −7.042 | −9.846 | 1.00 | 12.00 | C |
| ATOM | 3 | CB | ILE | A | 16 | 39.306 | −5.572 | −10.328 | 1.00 | 11.29 | C |
| ATOM | 4 | CG1 | ILE | A | 16 | 39.837 | −4.647 | −9.222 | 1.00 | 13.25 | C |
| ATOM | 5 | CD1 | ILE | A | 16 | 38.876 | −4.375 | −8.051 | 1.00 | 12.56 | C |
| ATOM | 6 | CG2 | ILE | A | 16 | 37.923 | −5.126 | −10.846 | 1.00 | 11.93 | C |
| ATOM | 7 | C | ILE | A | 16 | 38.850 | −7.930 | −11.043 | 1.00 | 12.38 | C |
| ATOM | 8 | O | ILE | A | 16 | 39.585 | −8.015 | −12.054 | 1.00 | 12.97 | O |
| ATOM | 9 | N | VAL | A | 17 | 37.677 | −8.526 | −10.928 | 1.00 | 11.14 | N |
| ATOM | 10 | CA | VAL | A | 17 | 37.116 | −9.388 | −11.971 | 1.00 | 12.08 | C |
| ATOM | 11 | CB | VAL | A | 17 | 36.434 | −10.659 | −11.355 | 1.00 | 11.46 | C |
| ATOM | 12 | CG1 | VAL | A | 17 | 35.746 | −11.544 | −12.452 | 1.00 | 9.73 | C |
| ATOM | 13 | CG2 | VAL | A | 17 | 37.473 | −11.472 | −10.484 | 1.00 | 10.55 | C |
| ATOM | 14 | C | VAL | A | 17 | 36.093 | −8.558 | −12.745 | 1.00 | 12.12 | C |
| ATOM | 15 | O | VAL | A | 17 | 35.212 | −7.966 | −12.145 | 1.00 | 12.56 | O |
| ATOM | 16 | N | GLY | A | 18 | 36.234 | −8.481 | −14.062 | 1.00 | 12.62 | N |
| ATOM | 17 | CA | GLY | A | 18 | 35.203 | −7.870 | −14.884 | 1.00 | 13.10 | C |
| ATOM | 18 | C | GLY | A | 18 | 35.319 | −6.343 | −14.929 | 1.00 | 12.87 | C |
| ATOM | 19 | O | GLY | A | 18 | 34.376 | −5.669 | −15.387 | 1.00 | 13.57 | O |
| ATOM | 20 | N | GLY | A | 19 | 36.468 | −5.816 | −14.491 | 1.00 | 13.09 | N |
| ATOM | 21 | CA | GLY | A | 19 | 36.707 | −4.376 | −14.427 | 1.00 | 13.54 | C |
| ATOM | 22 | C | GLY | A | 19 | 37.378 | −3.923 | −15.723 | 1.00 | 13.57 | C |
| ATOM | 23 | O | GLY | A | 19 | 37.210 | −4.553 | −16.784 | 1.00 | 13.01 | O |
| ATOM | 24 | N | ARG | A | 20 | 38.122 | −2.822 | −15.626 | 1.00 | 13.44 | N |
| ATOM | 25 | CA | ARG | A | 20 | 38.848 | −2.255 | −16.768 | 1.00 | 14.57 | C |
| ATOM | 26 | CB | ARG | A | 20 | 37.985 | −1.204 | −17.494 | 1.00 | 13.64 | C |
| ATOM | 27 | CG | ARG | A | 20 | 37.691 | 0.037 | −16.647 | 1.00 | 14.79 | C |
| ATOM | 28 | CD | ARG | A | 20 | 36.976 | 1.197 | −17.352 | 1.00 | 14.04 | C |
| ATOM | 29 | NE | ARG | A | 20 | 36.531 | 2.141 | −16.329 | 1.00 | 13.05 | N |
| ATOM | 30 | CZ | ARG | A | 20 | 35.391 | 2.013 | −15.647 | 1.00 | 13.61 | C |
| ATOM | 31 | NH1 | ARG | A | 20 | 34.533 | 1.034 | −15.914 | 1.00 | 13.45 | N |
| ATOM | 32 | NH2 | ARG | A | 20 | 35.093 | 2.871 | −14.707 | 1.00 | 13.02 | N |
| ATOM | 33 | C | ARG | A | 20 | 40.123 | −1.597 | −16.247 | 1.00 | 15.16 | C |
| ATOM | 34 | O | ARG | A | 20 | 40.227 | −1.281 | −15.035 | 1.00 | 15.46 | O |
| ATOM | 35 | N | ARG | A | 21 | 41.081 | −1.367 | −17.138 | 1.00 | 16.16 | N |
| ATOM | 36 | CA | ARG | A | 21 | 42.274 | −0.615 | −16.784 | 1.00 | 17.54 | C |
| ATOM | 37 | CB | ARG | A | 21 | 43.239 | −0.555 | −17.954 | 1.00 | 18.55 | C |
| ATOM | 38 | CG | ARG | A | 21 | 44.029 | −1.792 | −18.159 | 1.00 | 22.77 | C |
| ATOM | 39 | CD | ARG | A | 21 | 44.956 | −1.682 | −19.382 | 1.00 | 30.02 | C |
| ATOM | 40 | NE | ARG | A | 21 | 45.067 | −2.979 | −20.034 | 1.00 | 36.43 | N |
| ATOM | 41 | CZ | ARG | A | 21 | 45.463 | −3.174 | −21.286 | 1.00 | 39.49 | C |
| ATOM | 42 | NH1 | ARG | A | 21 | 45.827 | −2.137 | −22.041 | 1.00 | 41.06 | N |
| ATOM | 43 | NH2 | ARG | A | 21 | 45.501 | −4.419 | −21.781 | 1.00 | 41.02 | N |
| ATOM | 44 | C | ARG | A | 21 | 41.925 | 0.801 | −16.399 | 1.00 | 16.54 | C |
| ATOM | 45 | O | ARG | A | 21 | 41.158 | 1.500 | −17.101 | 1.00 | 16.86 | O |
| ATOM | 46 | N | ALA | A | 22 | 42.482 | 1.225 | −15.280 | 1.00 | 17.10 | N |
| ATOM | 47 | CA | ALA | A | 22 | 42.427 | 2.654 | −14.909 | 1.00 | 16.79 | C |
| ATOM | 48 | CB | ALA | A | 22 | 42.873 | 2.845 | −13.474 | 1.00 | 16.31 | C |
| ATOM | 49 | C | ALA | A | 22 | 43.335 | 3.464 | −15.836 | 1.00 | 17.44 | C |
| ATOM | 50 | O | ALA | A | 22 | 44.387 | 2.966 | −16.299 | 1.00 | 16.12 | O |
| ATOM | 51 | N | ARG | A | 23 | 42.949 | 4.725 | −16.067 | 1.00 | 17.52 | N |
| ATOM | 52 | CA | ARG | A | 23 | 43.843 | 5.710 | −16.675 | 1.00 | 18.21 | C |
| ATOM | 53 | CB | ARG | A | 23 | 43.094 | 7.065 | −16.802 | 1.00 | 17.37 | C |
| ATOM | 54 | CG | ARG | A | 23 | 43.984 | 8.251 | −17.284 | 1.00 | 21.55 | C |
| ATOM | 55 | CD | ARG | A | 23 | 43.222 | 9.559 | −17.611 | 1.00 | 20.04 | C |
| ATOM | 56 | NE | ARG | A | 23 | 42.231 | 9.848 | −16.566 | 1.00 | 28.14 | N |
| ATOM | 57 | CZ | ARG | A | 23 | 42.450 | 10.549 | −15.457 | 1.00 | 28.64 | C |
| ATOM | 58 | NH1 | ARG | A | 23 | 43.641 | 11.103 | −15.201 | 1.00 | 31.82 | N |
| ATOM | 59 | NH2 | ARG | A | 23 | 41.459 | 10.716 | −14.592 | 1.00 | 30.13 | N |
| ATOM | 60 | C | ARG | A | 23 | 45.072 | 5.846 | −15.767 | 1.00 | 18.42 | C |
| ATOM | 61 | O | ARG | A | 23 | 44.913 | 5.887 | −14.537 | 1.00 | 18.22 | O |
| ATOM | 62 | N | PRO | A | 24 | 46.290 | 5.913 | −16.329 | 1.00 | 19.26 | N |
| ATOM | 63 | CA | PRO | A | 24 | 47.480 | 5.938 | −15.486 | 1.00 | 19.02 | C |
| ATOM | 64 | CB | PRO | A | 24 | 48.628 | 6.155 | −16.490 | 1.00 | 19.69 | C |
| ATOM | 65 | CG | PRO | A | 24 | 48.069 | 5.615 | −17.834 | 1.00 | 19.59 | C |
| ATOM | 66 | CD | PRO | A | 24 | 46.620 | 5.970 | −17.774 | 1.00 | 18.88 | C |
| ATOM | 67 | C | PRO | A | 24 | 47.348 | 7.076 | −14.480 | 1.00 | 19.21 | C |
| ATOM | 68 | O | PRO | A | 24 | 47.008 | 8.191 | −14.853 | 1.00 | 18.93 | O |
| ATOM | 69 | N | HIS | A | 25 | 47.520 | 6.761 | −13.190 | 1.00 | 19.76 | N |
| ATOM | 70 | CA | HIS | A | 25 | 47.512 | 7.754 | −12.105 | 1.00 | 18.82 | C |
| ATOM | 71 | CB | HIS | A | 25 | 48.742 | 8.665 | −12.221 | 1.00 | 20.47 | C |
| ATOM | 72 | CG | HIS | A | 25 | 50.016 | 7.895 | −12.445 | 1.00 | 19.28 | C |
| ATOM | 73 | ND1 | HIS | A | 25 | 50.686 | 7.893 | −13.650 | 1.00 | 21.52 | N |
| ATOM | 74 | CE1 | HIS | A | 25 | 51.752 | 7.113 | −13.561 | 1.00 | 18.07 | C |
| ATOM | 75 | NE2 | HIS | A | 25 | 51.797 | 6.612 | −12.338 | 1.00 | 22.23 | N |
| ATOM | 76 | CD2 | HIS | A | 25 | 50.716 | 7.075 | −11.624 | 1.00 | 19.82 | C |
| ATOM | 77 | C | HIS | A | 25 | 46.212 | 8.550 | −11.934 | 1.00 | 18.43 | C |
| ATOM | 78 | O | HIS | A | 25 | 46.210 | 9.657 | −11.396 | 1.00 | 18.04 | O |
| ATOM | 79 | N | ALA | A | 26 | 45.098 | 7.948 | −12.337 | 1.00 | 17.50 | N |
| ATOM | 80 | CA | ALA | A | 26 | 43.771 | 8.509 | −12.080 | 1.00 | 16.85 | C |
| ATOM | 81 | CB | ALA | A | 26 | 42.735 | 7.680 | −12.767 | 1.00 | 17.64 | C |
| ATOM | 82 | C | ALA | A | 26 | 43.420 | 8.600 | −10.594 | 1.00 | 16.51 | C |
| ATOM | 83 | O | ALA | A | 26 | 42.660 | 9.475 | −10.213 | 1.00 | 15.45 | O |
| ATOM | 84 | N | TRP | A | 27 | 43.942 | 7.643 | −9.796 | 1.00 | 15.12 | N |
| ATOM | 85 | CA | TRP | A | 27 | 43.653 | 7.472 | −8.381 | 1.00 | 14.69 | C |
| ATOM | 86 | CB | TRP | A | 27 | 42.950 | 6.104 | −8.172 | 1.00 | 14.45 | C |
| ATOM | 87 | CG | TRP | A | 27 | 41.880 | 5.925 | −9.243 | 1.00 | 14.44 | C |
| ATOM | 88 | CD1 | TRP | A | 27 | 41.811 | 4.956 | −10.219 | 1.00 | 13.44 | C |
| ATOM | 89 | NE1 | TRP | A | 27 | 40.678 | 5.150 | −10.984 | 1.00 | 14.26 | N |
| ATOM | 90 | CE2 | TRP | A | 27 | 40.016 | 6.272 | −10.543 | 1.00 | 14.05 | C |
| ATOM | 91 | CD2 | TRP | A | 27 | 40.732 | 6.778 | −9.442 | 1.00 | 14.58 | C |
| ATOM | 92 | CE3 | TRP | A | 27 | 40.234 | 7.934 | −8.776 | 1.00 | 17.04 | C |
| ATOM | 93 | CZ3 | TRP | A | 27 | 39.061 | 8.553 | −9.262 | 1.00 | 15.90 | C |
| ATOM | 94 | CH2 | TRP | A | 27 | 38.383 | 8.027 | −10.380 | 1.00 | 15.49 | C |
| ATOM | 95 | CZ2 | TRP | A | 27 | 38.835 | 6.896 | −11.035 | 1.00 | 17.31 | C |
| ATOM | 96 | C | TRP | A | 27 | 44.968 | 7.580 | −7.600 | 1.00 | 14.64 | C |
| ATOM | 97 | O | TRP | A | 27 | 45.481 | 6.565 | −7.110 | 1.00 | 15.02 | O |
| ATOM | 98 | N | PRO | A | 28 | 45.531 | 8.794 | −7.496 | 1.00 | 14.24 | N |
| ATOM | 99 | CA | PRO | A | 28 | 46.888 | 8.986 | −6.947 | 1.00 | 13.76 | C |
| ATOM | 100 | CB | PRO | A | 28 | 47.213 | 10.465 | −7.280 | 1.00 | 12.97 | C |
| ATOM | 101 | CG | PRO | A | 28 | 45.931 | 11.131 | −7.512 | 1.00 | 14.41 | C |
| ATOM | 102 | CD | PRO | A | 28 | 44.879 | 10.082 | −7.826 | 1.00 | 15.00 | C |
| ATOM | 103 | C | PRO | A | 28 | 47.032 | 8.707 | −5.440 | 1.00 | 13.14 | C |
| ATOM | 104 | O | PRO | A | 28 | 48.170 | 8.635 | −4.961 | 1.00 | 13.54 | O |
| ATOM | 105 | N | PHE | A | 29 | 45.910 | 8.544 | −4.736 | 1.00 | 12.43 | N |
| ATOM | 106 | CA | PHE | A | 29 | 45.864 | 8.011 | −3.355 | 1.00 | 12.26 | C |
| ATOM | 107 | CB | PHE | A | 29 | 44.550 | 8.425 | −2.713 | 1.00 | 12.43 | C |
| ATOM | 108 | CG | PHE | A | 29 | 43.328 | 8.073 | −3.542 | 1.00 | 13.78 | C |
| ATOM | 109 | CD1 | PHE | A | 29 | 42.822 | 6.749 | −3.560 | 1.00 | 15.71 | C |
| ATOM | 110 | CE1 | PHE | A | 29 | 41.698 | 6.400 | −4.371 | 1.00 | 13.38 | C |
| ATOM | 111 | CZ | PHE | A | 29 | 41.093 | 7.387 | −5.170 | 1.00 | 13.27 | C |
| ATOM | 112 | CE2 | PHE | A | 29 | 41.605 | 8.725 | −5.155 | 1.00 | 13.16 | C |
| ATOM | 113 | CD2 | PHE | A | 29 | 42.702 | 9.051 | −4.344 | 1.00 | 15.57 | C |
| ATOM | 114 | C | PHE | A | 29 | 46.022 | 6.438 | −3.265 | 1.00 | 12.70 | C |
| ATOM | 115 | O | PHE | A | 29 | 46.172 | 5.877 | −2.177 | 1.00 | 13.41 | O |
| ATOM | 116 | N | MET | A | 30 | 45.987 | 5.766 | −4.407 | 1.00 | 12.53 | N |
| ATOM | 117 | CA | MET | A | 30 | 46.002 | 4.293 | −4.466 | 1.00 | 12.69 | C |
| ATOM | 118 | CB | MET | A | 30 | 45.447 | 3.801 | −5.809 | 1.00 | 11.84 | C |
| ATOM | 119 | CG | MET | A | 30 | 45.490 | 2.252 | −6.026 | 1.00 | 11.19 | C |
| ATOM | 120 | SD | MET | A | 30 | 44.288 | 1.476 | −4.926 | 1.00 | 11.54 | S |
| ATOM | 121 | CE | MET | A | 30 | 44.875 | −0.294 | −4.929 | 1.00 | 11.86 | C |
| ATOM | 122 | C | MET | A | 30 | 47.402 | 3.763 | −4.204 | 1.00 | 12.10 | C |
| ATOM | 123 | O | MET | A | 30 | 48.355 | 4.218 | −4.825 | 1.00 | 13.60 | O |
| ATOM | 124 | N | VAL | A | 31 | 47.518 | 2.779 | −3.302 | 1.00 | 12.14 | N |
| ATOM | 125 | CA | VAL | A | 31 | 48.798 | 2.275 | −2.820 | 1.00 | 12.04 | C |
| ATOM | 126 | CB | VAL | A | 31 | 48.889 | 2.473 | −1.256 | 1.00 | 11.65 | C |
| ATOM | 127 | CG1 | VAL | A | 31 | 50.167 | 1.921 | −0.726 | 1.00 | 11.59 | C |
| ATOM | 128 | CG2 | VAL | A | 31 | 48.717 | 3.945 | −0.866 | 1.00 | 12.60 | C |
| ATOM | 129 | C | VAL | A | 31 | 48.910 | 0.762 | −3.060 | 1.00 | 13.00 | C |
| ATOM | 130 | O | VAL | A | 31 | 47.903 | 0.053 | −2.896 | 1.00 | 11.94 | O |
| ATOM | 131 | N | SER | A | 32 | 50.103 | 0.283 | −3.445 | 1.00 | 11.98 | N |
| ATOM | 132 | CA | SER | A | 32 | 50.340 | −1.143 | −3.542 | 1.00 | 13.87 | C |
| ATOM | 133 | CB | SER | A | 32 | 51.054 | −1.544 | −4.847 | 1.00 | 13.37 | C |
| ATOM | 134 | OG | SER | A | 32 | 51.492 | −2.910 | −4.782 | 1.00 | 12.43 | O |
| ATOM | 135 | C | SER | A | 32 | 51.207 | −1.523 | −2.346 | 1.00 | 15.66 | C |
| ATOM | 136 | O | SER | A | 32 | 52.266 | −0.914 | −2.125 | 1.00 | 15.28 | O |
| ATOM | 137 | N | LEU | A | 33 | 50.733 | −2.503 | −1.575 | 1.00 | 15.75 | N |
| ATOM | 138 | CA | LEU | A | 33 | 51.474 | −3.053 | −0.446 | 1.00 | 17.43 | C |
| ATOM | 139 | CB | LEU | A | 33 | 50.491 | −3.448 | 0.677 | 1.00 | 17.27 | C |
| ATOM | 140 | CG | LEU | A | 33 | 50.237 | −2.587 | 1.923 | 1.00 | 19.97 | C |
| ATOM | 141 | CD1 | LEU | A | 33 | 50.680 | −1.104 | 1.851 | 1.00 | 17.93 | C |
| ATOM | 142 | CD2 | LEU | A | 33 | 48.840 | −2.700 | 2.463 | 1.00 | 19.09 | C |
| ATOM | 143 | C | LEU | A | 33 | 52.180 | −4.308 | −0.999 | 1.00 | 17.68 | C |
| ATOM | 144 | O | LEU | A | 33 | 51.536 | −5.171 | −1.678 | 1.00 | 15.85 | O |
| ATOM | 145 | N | GLN | A | 34 | 53.487 | −4.392 | −0.743 | 1.00 | 18.38 | N |
| ATOM | 146 | CA | GLN | A | 34 | 54.344 | −5.353 | −1.429 | 1.00 | 19.41 | C |
| ATOM | 147 | CB | GLN | A | 34 | 55.126 | −4.619 | −2.512 | 1.00 | 19.06 | C |
| ATOM | 148 | CG | GLN | A | 34 | 54.233 | −3.968 | −3.580 | 1.00 | 18.63 | C |
| ATOM | 149 | CD | GLN | A | 34 | 54.925 | −3.775 | −4.907 | 1.00 | 20.71 | C |
| ATOM | 150 | OE1 | GLN | A | 34 | 54.296 | −3.333 | −5.891 | 1.00 | 19.73 | O |
| ATOM | 151 | NE2 | GLN | A | 34 | 56.214 | −4.099 | −4.956 | 1.00 | 19.29 | N |
| ATOM | 152 | C | GLN | A | 34 | 55.324 | −6.073 | −0.472 | 1.00 | 21.22 | C |
| ATOM | 153 | O | GLN | A | 34 | 55.756 | −5.505 | 0.526 | 1.00 | 21.66 | O |
| ATOM | 154 | N | LEU | A | 35 | 55.621 | −7.337 | −0.746 | 1.00 | 22.46 | N |
| ATOM | 155 | CA | LEU | A | 35 | 56.675 | −8.025 | −0.001 | 1.00 | 24.57 | C |
| ATOM | 156 | CB | LEU | A | 35 | 56.136 | −9.254 | 0.744 | 1.00 | 23.24 | C |
| ATOM | 157 | CG | LEU | A | 35 | 55.222 | −8.912 | 1.934 | 1.00 | 23.62 | C |
| ATOM | 158 | CD1 | LEU | A | 35 | 54.360 | −10.097 | 2.423 | 1.00 | 20.48 | C |
| ATOM | 159 | CD2 | LEU | A | 35 | 55.974 | −8.277 | 3.127 | 1.00 | 22.73 | C |
| ATOM | 160 | C | LEU | A | 35 | 57.750 | −8.346 | −1.048 | 1.00 | 26.63 | C |
| ATOM | 161 | O | LEU | A | 35 | 57.555 | −8.032 | −2.225 | 1.00 | 26.96 | O |
| ATOM | 162 | N | ARG | A | 36 | 58.898 | −8.894 | −0.645 | 1.00 | 29.44 | N |
| ATOM | 163 | CA | ARG | A | 36 | 59.917 | −9.325 | −1.625 | 1.00 | 31.40 | C |
| ATOM | 164 | CB | ARG | A | 36 | 60.953 | −10.248 | −0.941 | 1.00 | 32.00 | C |
| ATOM | 165 | CG | ARG | A | 36 | 61.368 | −11.571 | −1.605 | 1.00 | 35.61 | C |
| ATOM | 166 | CD | ARG | A | 36 | 62.472 | −12.363 | −0.744 | 1.00 | 38.47 | C |
| ATOM | 167 | NE | ARG | A | 36 | 61.897 | −13.238 | 0.309 | 1.00 | 39.08 | N |
| ATOM | 168 | CZ | ARG | A | 36 | 61.721 | −12.915 | 1.610 | 1.00 | 41.12 | C |
| ATOM | 169 | NH1 | ARG | A | 36 | 62.074 | −11.712 | 2.079 | 1.00 | 40.47 | N |
| ATOM | 170 | NH2 | ARG | A | 36 | 61.173 | −13.804 | 2.454 | 1.00 | 40.66 | N |
| ATOM | 171 | C | ARG | A | 36 | 59.186 | −9.976 | −2.791 | 1.00 | 32.03 | C |
| ATOM | 172 | O | ARG | A | 36 | 58.300 | −10.824 | −2.594 | 1.00 | 34.04 | O |
| ATOM | 173 | N | GLY | A | 38 | 59.441 | −9.534 | −4.012 | 1.00 | 31.32 | N |
| ATOM | 174 | CA | GLY | A | 38 | 58.624 | −10.077 | −5.081 | 1.00 | 31.72 | C |
| ATOM | 175 | C | GLY | A | 38 | 57.082 | −9.996 | −4.874 | 1.00 | 31.18 | C |
| ATOM | 176 | O | GLY | A | 38 | 56.343 | −10.986 | −5.070 | 1.00 | 31.89 | O |
| ATOM | 177 | N | GLY | A | 39 | 56.599 | −8.818 | −4.460 | 1.00 | 28.45 | N |
| ATOM | 178 | CA | GLY | A | 39 | 55.395 | −8.314 | −5.096 | 1.00 | 24.99 | C |
| ATOM | 179 | C | GLY | A | 39 | 54.165 | −7.940 | −4.313 | 1.00 | 21.55 | C |
| ATOM | 180 | O | GLY | A | 39 | 54.074 | −8.191 | −3.134 | 1.00 | 20.73 | O |
| ATOM | 181 | N | HIS | A | 40 | 53.224 | −7.331 | −5.032 | 1.00 | 20.15 | N |
| ATOM | 182 | CA | HIS | A | 40 | 51.917 | −6.935 | −4.522 | 1.00 | 17.77 | C |
| ATOM | 183 | CB | HIS | A | 40 | 51.064 | −6.406 | −5.683 | 1.00 | 17.88 | C |
| ATOM | 184 | CG | HIS | A | 40 | 49.740 | −5.826 | −5.264 | 1.00 | 16.76 | C |
| ATOM | 185 | ND1 | HIS | A | 40 | 48.576 | −6.573 | −5.188 | 1.00 | 16.80 | N |
| ATOM | 186 | CE1 | HIS | A | 40 | 47.586 | −5.803 | −4.768 | 1.00 | 11.09 | C |
| ATOM | 187 | NE2 | HIS | A | 40 | 48.064 | −4.585 | −4.569 | 1.00 | 16.24 | N |
| ATOM | 188 | CD2 | HIS | A | 40 | 49.401 | −4.569 | −4.894 | 1.00 | 14.30 | C |
| ATOM | 189 | C | HIS | A | 40 | 51.167 | −8.070 | −3.769 | 1.00 | 17.06 | C |
| ATOM | 190 | O | HIS | A | 40 | 50.979 | −9.182 | −4.290 | 1.00 | 16.92 | O |
| ATOM | 191 | N | PHE | A | 41 | 50.696 | −7.776 | −2.564 | 1.00 | 15.59 | N |
| ATOM | 192 | CA | PHE | A | 41 | 49.743 | −8.679 | −1.888 | 1.00 | 14.92 | C |
| ATOM | 193 | CB | PHE | A | 41 | 50.431 | −9.425 | −0.714 | 1.00 | 14.62 | C |
| ATOM | 194 | CG | PHE | A | 41 | 50.812 | −8.506 | 0.441 | 1.00 | 14.60 | C |
| ATOM | 195 | CD1 | PHE | A | 41 | 49.952 | −8.327 | 1.523 | 1.00 | 12.63 | C |
| ATOM | 196 | CE1 | PHE | A | 41 | 50.283 | −7.472 | 2.587 | 1.00 | 14.17 | C |
| ATOM | 197 | CZ | PHE | A | 41 | 51.462 | −6.736 | 2.558 | 1.00 | 10.65 | C |
| ATOM | 198 | CE2 | PHE | A | 41 | 52.337 | −6.904 | 1.483 | 1.00 | 14.85 | C |
| ATOM | 199 | CD2 | PHE | A | 41 | 51.997 | −7.778 | 0.409 | 1.00 | 14.23 | C |
| ATOM | 200 | C | PHE | A | 41 | 48.466 | −7.974 | −1.389 | 1.00 | 14.40 | C |
| ATOM | 201 | O | PHE | A | 41 | 47.506 | −8.648 | −0.991 | 1.00 | 14.01 | O |
| ATOM | 202 | N | CYS | A | 42 | 48.437 | −6.632 | −1.388 | 1.00 | 13.05 | N |
| ATOM | 203 | CA | CYS | A | 42 | 47.243 | −5.917 | −0.879 | 1.00 | 13.06 | C |
| ATOM | 204 | CB | CYS | A | 42 | 47.193 | −5.949 | 0.654 | 1.00 | 12.32 | C |
| ATOM | 205 | SG | CYS | A | 42 | 46.270 | −7.337 | 1.394 | 1.00 | 13.76 | S |
| ATOM | 206 | C | CYS | A | 42 | 47.259 | −4.450 | −1.317 | 1.00 | 13.02 | C |
| ATOM | 207 | O | CYS | A | 42 | 48.334 | −3.932 | −1.613 | 1.00 | 13.82 | O |
| ATOM | 208 | N | GLY | A | 43 | 46.091 | −3.800 | −1.316 | 1.00 | 12.15 | N |
| ATOM | 209 | CA | GLY | A | 43 | 46.008 | −2.375 | −1.612 | 1.00 | 10.37 | C |
| ATOM | 210 | C | GLY | A | 43 | 46.056 | −1.597 | −0.314 | 1.00 | 11.19 | C |
| ATOM | 211 | O | GLY | A | 43 | 45.983 | −2.188 | 0.778 | 1.00 | 10.67 | O |
| ATOM | 212 | N | ALA | A | 44 | 46.113 | −0.274 | −0.434 | 1.00 | 10.11 | N |
| ATOM | 213 | CA | ALA | A | 44 | 45.984 | 0.606 | 0.697 | 1.00 | 11.86 | C |
| ATOM | 214 | CB | ALA | A | 44 | 47.289 | 0.680 | 1.577 | 1.00 | 10.69 | C |
| ATOM | 215 | C | ALA | A | 44 | 45.634 | 1.965 | 0.103 | 1.00 | 12.00 | C |
| ATOM | 216 | O | ALA | A | 44 | 45.508 | 2.082 | −1.121 | 1.00 | 11.65 | O |
| ATOM | 217 | N | THR | A | 45 | 45.370 | 2.934 | 0.982 | 1.00 | 12.30 | N |
| ATOM | 218 | CA | THR | A | 45 | 45.005 | 4.296 | 0.587 | 1.00 | 12.81 | C |
| ATOM | 219 | CB | THR | A | 45 | 43.550 | 4.602 | 1.014 | 1.00 | 13.44 | C |
| ATOM | 220 | OG1 | THR | A | 45 | 42.663 | 3.716 | 0.348 | 1.00 | 10.80 | O |
| ATOM | 221 | CG2 | THR | A | 45 | 43.091 | 6.063 | 0.532 | 1.00 | 11.92 | C |
| ATOM | 222 | C | THR | A | 45 | 45.894 | 5.272 | 1.340 | 1.00 | 12.56 | C |
| ATOM | 223 | O | THR | A | 45 | 45.967 | 5.194 | 2.588 | 1.00 | 11.87 | O |
| ATOM | 224 | N | LEU | A | 46 | 46.506 | 6.213 | 0.611 | 1.00 | 12.36 | N |
| ATOM | 225 | CA | LEU | A | 46 | 47.313 | 7.247 | 1.231 | 1.00 | 13.48 | C |
| ATOM | 226 | CB | LEU | A | 46 | 48.220 | 7.960 | 0.198 | 1.00 | 13.84 | C |
| ATOM | 227 | CG | LEU | A | 46 | 49.223 | 8.949 | 0.817 | 1.00 | 13.04 | C |
| ATOM | 228 | CD1 | LEU | A | 46 | 50.300 | 8.225 | 1.667 | 1.00 | 12.07 | C |
| ATOM | 229 | CD2 | LEU | A | 46 | 49.825 | 9.840 | −0.270 | 1.00 | 13.75 | C |
| ATOM | 230 | C | LEU | A | 46 | 46.362 | 8.243 | 1.918 | 1.00 | 14.51 | C |
| ATOM | 231 | O | LEU | A | 46 | 45.511 | 8.860 | 1.260 | 1.00 | 15.02 | O |
| ATOM | 232 | N | ILE | A | 47 | 46.437 | 8.338 | 3.240 | 1.00 | 14.25 | N |
| ATOM | 233 | CA | ILE | A | 47 | 45.484 | 9.202 | 3.976 | 1.00 | 14.41 | C |
| ATOM | 234 | CB | ILE | A | 47 | 44.672 | 8.416 | 5.045 | 1.00 | 14.20 | C |
| ATOM | 235 | CG1 | ILE | A | 47 | 45.622 | 7.678 | 6.018 | 1.00 | 14.79 | C |
| ATOM | 236 | CD1 | ILE | A | 47 | 44.914 | 7.146 | 7.282 | 1.00 | 13.54 | C |
| ATOM | 237 | CG2 | ILE | A | 47 | 43.582 | 7.480 | 4.349 | 1.00 | 15.52 | C |
| ATOM | 238 | C | ILE | A | 47 | 46.071 | 10.475 | 4.618 | 1.00 | 14.56 | C |
| ATOM | 239 | O | ILE | A | 47 | 45.307 | 11.285 | 5.193 | 1.00 | 14.09 | O |
| ATOM | 240 | N | ALA | A | 48 | 47.392 | 10.594 | 4.521 | 1.00 | 14.19 | N |
| ATOM | 241 | CA | ALA | A | 48 | 48.219 | 11.738 | 4.951 | 1.00 | 15.67 | C |
| ATOM | 242 | CB | ALA | A | 48 | 48.311 | 11.788 | 6.459 | 1.00 | 15.65 | C |
| ATOM | 243 | C | ALA | A | 48 | 49.602 | 11.428 | 4.342 | 1.00 | 16.43 | C |
| ATOM | 244 | O | ALA | A | 48 | 49.859 | 10.262 | 3.973 | 1.00 | 16.71 | O |
| ATOM | 245 | N | PRO | A | 49 | 50.503 | 12.407 | 4.164 | 1.00 | 17.36 | N |
| ATOM | 246 | CA | PRO | A | 49 | 51.781 | 12.061 | 3.536 | 1.00 | 16.82 | C |
| ATOM | 247 | CB | PRO | A | 49 | 52.576 | 13.371 | 3.577 | 1.00 | 17.94 | C |
| ATOM | 248 | CG | PRO | A | 49 | 51.501 | 14.448 | 3.636 | 1.00 | 17.95 | C |
| ATOM | 249 | CD | PRO | A | 49 | 50.408 | 13.860 | 4.477 | 1.00 | 17.23 | C |
| ATOM | 250 | C | PRO | A | 49 | 52.520 | 10.901 | 4.267 | 1.00 | 17.44 | C |
| ATOM | 251 | O | PRO | A | 49 | 53.272 | 10.181 | 3.590 | 1.00 | 17.55 | O |
| ATOM | 252 | N | ASN | A | 50 | 52.297 | 10.681 | 5.566 | 1.00 | 16.71 | N |
| ATOM | 253 | CA | ASN | A | 50 | 53.051 | 9.622 | 6.230 | 1.00 | 18.36 | C |
| ATOM | 254 | CB | ASN | A | 50 | 53.996 | 10.201 | 7.303 | 1.00 | 19.25 | C |
| ATOM | 255 | CG | ASN | A | 50 | 53.269 | 11.047 | 8.313 | 1.00 | 20.90 | C |
| ATOM | 256 | OD1 | ASN | A | 50 | 52.276 | 11.719 | 7.983 | 1.00 | 25.72 | O |
| ATOM | 257 | ND2 | ASN | A | 50 | 53.729 | 11.005 | 9.559 | 1.00 | 22.83 | N |
| ATOM | 258 | C | ASN | A | 50 | 52.184 | 8.507 | 6.843 | 1.00 | 18.26 | C |
| ATOM | 259 | O | ASN | A | 50 | 52.648 | 7.793 | 7.763 | 1.00 | 17.93 | O |
| ATOM | 260 | N | PHE | A | 51 | 50.939 | 8.393 | 6.364 | 1.00 | 17.57 | N |
| ATOM | 261 | CA | PHE | A | 51 | 50.036 | 7.316 | 6.768 | 1.00 | 17.25 | C |
| ATOM | 262 | CB | PHE | A | 51 | 49.007 | 7.785 | 7.804 | 1.00 | 18.10 | C |
| ATOM | 263 | CG | PHE | A | 51 | 49.586 | 8.248 | 9.141 | 1.00 | 19.62 | C |
| ATOM | 264 | CD1 | PHE | A | 51 | 49.657 | 7.370 | 10.234 | 1.00 | 21.93 | C |
| ATOM | 265 | CE1 | PHE | A | 51 | 50.138 | 7.817 | 11.509 | 1.00 | 23.91 | C |
| ATOM | 266 | CZ | PHE | A | 51 | 50.528 | 9.150 | 11.672 | 1.00 | 21.77 | C |
| ATOM | 267 | CE2 | PHE | A | 51 | 50.434 | 10.061 | 10.574 | 1.00 | 20.23 | C |
| ATOM | 268 | CD2 | PHE | A | 51 | 49.957 | 9.605 | 9.334 | 1.00 | 22.64 | C |
| ATOM | 269 | C | PHE | A | 51 | 49.242 | 6.767 | 5.576 | 1.00 | 16.78 | C |
| ATOM | 270 | O | PHE | A | 51 | 48.773 | 7.527 | 4.715 | 1.00 | 14.17 | O |
| ATOM | 271 | N | VAL | A | 52 | 49.083 | 5.436 | 5.563 | 1.00 | 15.88 | N |
| ATOM | 272 | CA | VAL | A | 52 | 48.184 | 4.752 | 4.650 | 1.00 | 15.38 | C |
| ATOM | 273 | CB | VAL | A | 52 | 48.915 | 3.830 | 3.619 | 1.00 | 14.90 | C |
| ATOM | 274 | CG1 | VAL | A | 52 | 49.638 | 2.669 | 4.314 | 1.00 | 15.67 | C |
| ATOM | 275 | CG2 | VAL | A | 52 | 49.859 | 4.628 | 2.778 | 1.00 | 12.90 | C |
| ATOM | 276 | C | VAL | A | 52 | 47.243 | 3.947 | 5.501 | 1.00 | 15.78 | C |
| ATOM | 277 | O | VAL | A | 52 | 47.576 | 3.567 | 6.654 | 1.00 | 15.67 | O |
| ATOM | 278 | N | MET | A | 53 | 46.044 | 3.725 | 4.989 | 1.00 | 15.72 | N |
| ATOM | 279 | CA | MET | A | 53 | 45.158 | 2.787 | 5.651 | 1.00 | 15.09 | C |
| ATOM | 280 | CB | MET | A | 53 | 43.897 | 3.460 | 6.201 | 1.00 | 16.54 | C |
| ATOM | 281 | CG | MET | A | 53 | 42.794 | 3.690 | 5.218 | 1.00 | 16.68 | C |
| ATOM | 282 | SD | MET | A | 53 | 41.290 | 4.559 | 5.835 | 1.00 | 19.62 | S |
| ATOM | 283 | CE | MET | A | 53 | 40.646 | 4.832 | 4.192 | 1.00 | 16.12 | C |
| ATOM | 284 | C | MET | A | 53 | 44.832 | 1.589 | 4.736 | 1.00 | 13.63 | C |
| ATOM | 285 | O | MET | A | 53 | 44.753 | 1.707 | 3.517 | 1.00 | 14.02 | O |
| ATOM | 286 | N | SER | A | 54 | 44.622 | 0.449 | 5.356 | 1.00 | 12.58 | N |
| ATOM | 287 | CA | SER | A | 54 | 44.437 | −0.791 | 4.641 | 1.00 | 12.17 | C |
| ATOM | 288 | CB | SER | A | 54 | 45.817 | −1.415 | 4.382 | 1.00 | 12.78 | C |
| ATOM | 289 | OG | SER | A | 54 | 45.699 | −2.489 | 3.449 | 1.00 | 12.44 | O |
| ATOM | 290 | C | SER | A | 54 | 43.552 | −1.708 | 5.499 | 1.00 | 12.76 | C |
| ATOM | 291 | O | SER | A | 54 | 43.025 | −1.264 | 6.533 | 1.00 | 12.21 | O |
| ATOM | 292 | N | ALA | A | 55 | 43.330 | −2.940 | 5.042 | 1.00 | 12.24 | N |
| ATOM | 293 | CA | ALA | A | 55 | 42.562 | −3.920 | 5.801 | 1.00 | 11.99 | C |
| ATOM | 294 | CB | ALA | A | 55 | 41.871 | −4.939 | 4.887 | 1.00 | 11.04 | C |
| ATOM | 295 | C | ALA | A | 55 | 43.468 | −4.622 | 6.807 | 1.00 | 12.87 | C |
| ATOM | 296 | O | ALA | A | 55 | 44.618 | −4.966 | 6.500 | 1.00 | 10.74 | O |
| ATOM | 297 | N | ALA | A | 56 | 42.944 | −4.821 | 8.019 | 1.00 | 13.43 | N |
| ATOM | 298 | CA | ALA | A | 56 | 43.733 | −5.504 | 9.048 | 1.00 | 14.08 | C |
| ATOM | 299 | CB | ALA | A | 56 | 42.991 | −5.546 | 10.335 | 1.00 | 13.99 | C |
| ATOM | 300 | C | ALA | A | 56 | 44.129 | −6.921 | 8.607 | 1.00 | 14.69 | C |
| ATOM | 301 | O | ALA | A | 56 | 45.257 | −7.364 | 8.886 | 1.00 | 14.68 | O |
| ATOM | 302 | N | HIS | A | 57 | 43.219 | −7.606 | 7.907 | 1.00 | 15.16 | N |
| ATOM | 303 | CA | HIS | A | 57 | 43.508 | −8.951 | 7.391 | 1.00 | 16.41 | C |
| ATOM | 304 | CB | HIS | A | 57 | 42.229 | −9.653 | 6.847 | 1.00 | 16.15 | C |
| ATOM | 305 | CG | HIS | A | 57 | 41.885 | −9.318 | 5.421 | 1.00 | 16.77 | C |
| ATOM | 306 | ND1 | HIS | A | 57 | 40.941 | −8.367 | 5.089 | 1.00 | 15.58 | N |
| ATOM | 307 | CE1 | HIS | A | 57 | 40.841 | −8.290 | 3.771 | 1.00 | 16.63 | C |
| ATOM | 308 | NE2 | HIS | A | 57 | 41.666 | −9.175 | 3.241 | 1.00 | 15.61 | N |
| ATOM | 309 | CD2 | HIS | A | 57 | 42.342 | −9.817 | 4.249 | 1.00 | 14.96 | C |
| ATOM | 310 | C | HIS | A | 57 | 44.725 | −9.009 | 6.425 | 1.00 | 16.75 | C |
| ATOM | 311 | O | HIS | A | 57 | 45.385 | −10.039 | 6.313 | 1.00 | 16.42 | O |
| ATOM | 312 | N | CYS | A | 58 | 45.029 | −7.900 | 5.753 | 1.00 | 16.20 | N |
| ATOM | 313 | CA | CYS | A | 58 | 46.251 | −7.810 | 4.940 | 1.00 | 17.05 | C |
| ATOM | 314 | CB | CYS | A | 58 | 46.350 | −6.462 | 4.234 | 1.00 | 15.60 | C |
| ATOM | 315 | SG | CYS | A | 58 | 45.217 | −6.409 | 2.862 | 1.00 | 12.44 | S |
| ATOM | 316 | C | CYS | A | 58 | 47.513 | −7.977 | 5.772 | 1.00 | 18.88 | C |
| ATOM | 317 | O | CYS | A | 58 | 48.482 | −8.517 | 5.278 | 1.00 | 19.27 | O |
| ATOM | 318 | N | VAL | A | 59 | 47.506 | −7.517 | 7.028 | 1.00 | 20.85 | N |
| ATOM | 319 | CA | VAL | A | 59 | 48.753 | −7.559 | 7.804 | 1.00 | 22.71 | C |
| ATOM | 320 | CB | VAL | A | 59 | 49.230 | −6.169 | 8.236 | 1.00 | 22.82 | C |
| ATOM | 321 | CG1 | VAL | A | 59 | 49.567 | −5.317 | 7.015 | 1.00 | 24.06 | C |
| ATOM | 322 | CG2 | VAL | A | 59 | 48.230 | −5.488 | 9.201 | 1.00 | 21.55 | C |
| ATOM | 323 | C | VAL | A | 59 | 48.794 | −8.505 | 9.002 | 1.00 | 24.98 | C |
| ATOM | 324 | O | VAL | A | 59 | 49.853 | −8.615 | 9.631 | 1.00 | 24.74 | O |
| ATOM | 325 | N | ALA | A | 60 | 47.670 | −9.191 | 9.289 | 1.00 | 27.02 | N |
| ATOM | 326 | CA | ALA | A | 60 | 47.518 | −10.111 | 10.438 | 1.00 | 29.91 | C |
| ATOM | 327 | CB | ALA | A | 60 | 46.190 | −10.872 | 10.339 | 1.00 | 29.91 | C |
| ATOM | 328 | C | ALA | A | 60 | 48.678 | −11.120 | 10.597 | 1.00 | 32.27 | C |
| ATOM | 329 | O | ALA | A | 60 | 49.202 | −11.340 | 11.716 | 1.00 | 34.03 | O |
| ATOM | 330 | N | ASN | A | 61 | 49.081 | −11.754 | 9.509 | 1.00 | 33.13 | N |
| ATOM | 331 | CA | ASN | A | 61 | 50.158 | −12.715 | 9.651 | 1.00 | 35.51 | C |
| ATOM | 332 | CB | ASN | A | 61 | 49.637 | −14.156 | 9.495 | 1.00 | 36.23 | C |
| ATOM | 333 | CG | ASN | A | 61 | 49.301 | −14.784 | 10.832 | 1.00 | 40.34 | C |
| ATOM | 334 | OD1 | ASN | A | 61 | 48.121 | −14.899 | 11.210 | 1.00 | 43.39 | O |
| ATOM | 335 | ND2 | ASN | A | 61 | 50.345 | −15.156 | 11.587 | 1.00 | 43.95 | N |
| ATOM | 336 | C | ASN | A | 61 | 51.348 | −12.457 | 8.761 | 1.00 | 35.19 | C |
| ATOM | 337 | O | ASN | A | 61 | 51.982 | −13.395 | 8.298 | 1.00 | 35.71 | O |
| ATOM | 338 | N | VAL | A | 62 | 51.645 | −11.184 | 8.511 | 1.00 | 35.06 | N |
| ATOM | 339 | CA | VAL | A | 62 | 52.827 | −10.829 | 7.730 | 1.00 | 35.13 | C |
| ATOM | 340 | CB | VAL | A | 62 | 52.493 | −9.971 | 6.455 | 1.00 | 35.21 | C |
| ATOM | 341 | CG1 | VAL | A | 62 | 51.060 | −10.238 | 5.964 | 1.00 | 36.22 | C |
| ATOM | 342 | CG2 | VAL | A | 62 | 52.720 | −8.506 | 6.697 | 1.00 | 35.54 | C |
| ATOM | 343 | C | VAL | A | 62 | 53.851 | −10.139 | 8.627 | 1.00 | 34.71 | C |
| ATOM | 344 | O | VAL | A | 62 | 53.491 | −9.544 | 9.638 | 1.00 | 34.35 | O |
| ATOM | 345 | N | ASN | A | 62A | 55.131 | −10.241 | 8.273 | 1.00 | 35.31 | N |
| ATOM | 346 | CA | ASN | A | 62A | 56.126 | −9.423 | 8.951 | 1.00 | 35.48 | C |
| ATOM | 347 | CB | ASN | A | 62A | 57.533 | −10.019 | 8.922 | 1.00 | 36.08 | C |
| ATOM | 348 | CG | ASN | A | 62A | 58.580 | −9.038 | 9.449 | 1.00 | 38.63 | C |
| ATOM | 349 | OD1 | ASN | A | 62A | 58.313 | −8.241 | 10.376 | 1.00 | 39.11 | O |
| ATOM | 350 | ND2 | ASN | A | 62A | 59.768 | −9.059 | 8.838 | 1.00 | 41.22 | N |
| ATOM | 351 | C | ASN | A | 62A | 56.118 | −8.027 | 8.342 | 1.00 | 34.59 | C |
| ATOM | 352 | O | ASN | A | 62A | 56.530 | −7.793 | 7.198 | 1.00 | 34.41 | O |
| ATOM | 353 | N | VAL | A | 62B | 55.647 | −7.113 | 9.160 | 1.00 | 34.36 | N |
| ATOM | 354 | CA | VAL | A | 62B | 55.374 | −5.747 | 8.772 | 1.00 | 33.98 | C |
| ATOM | 355 | CB | VAL | A | 62B | 54.500 | −5.127 | 9.855 | 1.00 | 34.11 | C |
| ATOM | 356 | CG1 | VAL | A | 62B | 54.469 | −3.626 | 9.779 | 1.00 | 33.58 | C |
| ATOM | 357 | CG2 | VAL | A | 62B | 53.094 | −5.690 | 9.725 | 1.00 | 34.32 | C |
| ATOM | 358 | C | VAL | A | 62B | 56.672 | −4.969 | 8.486 | 1.00 | 34.24 | C |
| ATOM | 359 | O | VAL | A | 62B | 56.676 | −4.033 | 7.678 | 1.00 | 33.61 | O |
| ATOM | 360 | N | ARG | A | 63 | 57.761 | −5.401 | 9.130 | 1.00 | 33.47 | N |
| ATOM | 361 | CA | ARG | A | 63 | 59.107 | −4.898 | 8.882 | 1.00 | 33.49 | C |
| ATOM | 362 | CB | ARG | A | 63 | 60.119 | −5.656 | 9.753 | 1.00 | 35.20 | C |
| ATOM | 363 | CG | ARG | A | 63 | 60.309 | −5.151 | 11.184 | 1.00 | 39.91 | C |
| ATOM | 364 | CD | ARG | A | 63 | 61.506 | −5.828 | 11.902 | 1.00 | 47.66 | C |
| ATOM | 365 | NE | ARG | A | 63 | 61.170 | −7.157 | 12.438 | 1.00 | 52.69 | N |
| ATOM | 366 | CZ | ARG | A | 63 | 61.495 | −8.319 | 11.855 | 1.00 | 55.17 | C |
| ATOM | 367 | NH1 | ARG | A | 63 | 62.167 | −8.335 | 10.706 | 1.00 | 56.40 | N |
| ATOM | 368 | NH2 | ARG | A | 63 | 61.138 | −9.468 | 12.420 | 1.00 | 56.03 | N |
| ATOM | 369 | C | ARG | A | 63 | 59.505 | −5.155 | 7.447 | 1.00 | 31.51 | C |
| ATOM | 370 | O | ARG | A | 63 | 60.395 | −4.486 | 6.922 | 1.00 | 30.84 | O |
| ATOM | 371 | N | ALA | A | 64 | 58.872 | −6.156 | 6.830 | 1.00 | 28.74 | N |
| ATOM | 372 | CA | ALA | A | 64 | 59.188 | −6.534 | 5.459 | 1.00 | 26.93 | C |
| ATOM | 373 | CB | ALA | A | 64 | 59.042 | −8.044 | 5.280 | 1.00 | 27.18 | C |
| ATOM | 374 | C | ALA | A | 64 | 58.357 | −5.783 | 4.402 | 1.00 | 25.29 | C |
| ATOM | 375 | O | ALA | A | 64 | 58.710 | −5.773 | 3.248 | 1.00 | 24.94 | O |
| ATOM | 376 | N | VAL | A | 65 | 57.265 | −5.151 | 4.817 | 1.00 | 24.68 | N |
| ATOM | 377 | CA | VAL | A | 65 | 56.325 | −4.549 | 3.885 | 1.00 | 22.98 | C |
| ATOM | 378 | CB | VAL | A | 65 | 54.982 | −4.243 | 4.557 | 1.00 | 22.60 | C |
| ATOM | 379 | CG1 | VAL | A | 65 | 54.043 | −3.510 | 3.568 | 1.00 | 21.02 | C |
| ATOM | 380 | CG2 | VAL | A | 65 | 54.341 | −5.530 | 5.076 | 1.00 | 19.48 | C |
| ATOM | 381 | C | VAL | A | 65 | 56.870 | −3.274 | 3.222 | 1.00 | 23.39 | C |
| ATOM | 382 | O | VAL | A | 65 | 57.315 | −2.349 | 3.909 | 1.00 | 22.89 | O |
| ATOM | 383 | N | ARG | A | 65A | 56.816 | −3.260 | 1.892 | 1.00 | 22.68 | N |
| ATOM | 384 | CA | ARG | A | 65A | 57.068 | −2.060 | 1.091 | 1.00 | 22.73 | C |
| ATOM | 385 | CB | ARG | A | 65A | 57.898 | −2.405 | −0.141 | 1.00 | 22.88 | C |
| ATOM | 386 | CG | ARG | A | 65A | 59.317 | −2.954 | 0.180 | 1.00 | 28.96 | C |
| ATOM | 387 | CD | ARG | A | 65A | 60.121 | −2.098 | 1.152 | 1.00 | 38.16 | C |
| ATOM | 388 | NE | ARG | A | 65A | 60.032 | −2.603 | 2.530 | 1.00 | 43.06 | N |
| ATOM | 389 | CZ | ARG | A | 65A | 60.959 | −2.429 | 3.475 | 1.00 | 44.40 | C |
| ATOM | 390 | NH1 | ARG | A | 65A | 62.092 | −1.764 | 3.220 | 1.00 | 46.12 | N |
| ATOM | 391 | NH2 | ARG | A | 65A | 60.757 | −2.950 | 4.677 | 1.00 | 43.61 | N |
| ATOM | 392 | C | ARG | A | 65A | 55.772 | −1.382 | 0.654 | 1.00 | 21.43 | C |
| ATOM | 393 | O | ARG | A | 65A | 54.882 | −2.018 | 0.061 | 1.00 | 19.48 | O |
| ATOM | 394 | N | VAL | A | 66 | 55.708 | −0.079 | 0.925 | 1.00 | 20.33 | N |
| ATOM | 395 | CA | VAL | A | 66 | 54.518 | 0.710 | 0.688 | 1.00 | 18.92 | C |
| ATOM | 396 | CB | VAL | A | 66 | 54.182 | 1.617 | 1.870 | 1.00 | 19.25 | C |
| ATOM | 397 | CG1 | VAL | A | 66 | 52.953 | 2.471 | 1.551 | 1.00 | 16.89 | C |
| ATOM | 398 | CG2 | VAL | A | 66 | 53.933 | 0.788 | 3.140 | 1.00 | 17.68 | C |
| ATOM | 399 | C | VAL | A | 66 | 54.859 | 1.533 | −0.536 | 1.00 | 19.67 | C |
| ATOM | 400 | O | VAL | A | 66 | 55.769 | 2.422 | −0.498 | 1.00 | 18.85 | O |
| ATOM | 401 | N | VAL | A | 67 | 54.165 | 1.201 | −1.623 | 1.00 | 17.67 | N |
| ATOM | 402 | CA | VAL | A | 67 | 54.493 | 1.725 | −2.925 | 1.00 | 16.96 | C |
| ATOM | 403 | CB | VAL | A | 67 | 54.669 | 0.556 | −3.970 | 1.00 | 16.97 | C |
| ATOM | 404 | CG1 | VAL | A | 67 | 54.957 | 1.081 | −5.377 | 1.00 | 16.82 | C |
| ATOM | 405 | CG2 | VAL | A | 67 | 55.790 | −0.399 | −3.532 | 1.00 | 14.97 | C |
| ATOM | 406 | C | VAL | A | 67 | 53.410 | 2.722 | −3.354 | 1.00 | 17.54 | C |
| ATOM | 407 | O | VAL | A | 67 | 52.254 | 2.356 | −3.620 | 1.00 | 17.59 | O |
| ATOM | 408 | N | LEU | A | 68 | 53.801 | 3.987 | −3.443 | 1.00 | 16.01 | N |
| ATOM | 409 | CA | LEU | A | 68 | 52.907 | 5.046 | −3.859 | 1.00 | 15.96 | C |
| ATOM | 410 | CB | LEU | A | 68 | 53.142 | 6.312 | −3.007 | 1.00 | 15.59 | C |
| ATOM | 411 | CG | LEU | A | 68 | 53.317 | 6.125 | −1.506 | 1.00 | 15.78 | C |
| ATOM | 412 | CD1 | LEU | A | 68 | 53.611 | 7.475 | −0.815 | 1.00 | 14.60 | C |
| ATOM | 413 | CD2 | LEU | A | 68 | 52.076 | 5.464 | −0.877 | 1.00 | 14.30 | C |
| ATOM | 414 | C | LEU | A | 68 | 53.209 | 5.382 | −5.290 | 1.00 | 15.81 | C |
| ATOM | 415 | O | LEU | A | 68 | 54.325 | 5.129 | −5.760 | 1.00 | 17.70 | O |
| ATOM | 416 | N | GLY | A | 69 | 52.248 | 5.982 | −5.976 | 1.00 | 15.34 | N |
| ATOM | 417 | CA | GLY | A | 69 | 52.463 | 6.490 | −7.329 | 1.00 | 16.68 | C |
| ATOM | 418 | C | GLY | A | 69 | 52.614 | 5.456 | −8.434 | 1.00 | 17.05 | C |
| ATOM | 419 | O | GLY | A | 69 | 53.127 | 5.786 | −9.506 | 1.00 | 17.27 | O |
| ATOM | 420 | N | ALA | A | 70 | 52.157 | 4.212 | −8.191 | 1.00 | 17.17 | N |
| ATOM | 421 | CA | ALA | A | 70 | 52.319 | 3.130 | −9.157 | 1.00 | 16.67 | C |
| ATOM | 422 | CB | ALA | A | 70 | 52.544 | 1.767 | −8.423 | 1.00 | 17.09 | C |
| ATOM | 423 | C | ALA | A | 70 | 51.116 | 3.025 | −10.093 | 1.00 | 17.50 | C |
| ATOM | 424 | O | ALA | A | 70 | 50.010 | 3.548 | −9.795 | 1.00 | 15.31 | O |
| ATOM | 425 | N | HIS | A | 71 | 51.337 | 2.392 | −11.247 | 1.00 | 17.08 | N |
| ATOM | 426 | CA | HIS | A | 71 | 50.211 | 2.110 | −12.150 | 1.00 | 18.08 | C |
| ATOM | 427 | CB | HIS | A | 71 | 50.122 | 3.109 | −13.305 | 1.00 | 17.20 | C |
| ATOM | 428 | CG | HIS | A | 71 | 49.016 | 2.806 | −14.271 | 1.00 | 17.53 | C |
| ATOM | 429 | ND1 | HIS | A | 71 | 47.689 | 2.798 | −13.905 | 1.00 | 15.14 | N |
| ATOM | 430 | CE1 | HIS | A | 71 | 46.950 | 2.490 | −14.950 | 1.00 | 17.77 | C |
| ATOM | 431 | NE2 | HIS | A | 71 | 47.753 | 2.286 | −15.983 | 1.00 | 17.97 | N |
| ATOM | 432 | CD2 | HIS | A | 71 | 49.049 | 2.471 | −15.583 | 1.00 | 15.71 | C |
| ATOM | 433 | C | HIS | A | 71 | 50.353 | 0.709 | −12.686 | 1.00 | 18.82 | C |
| ATOM | 434 | O | HIS | A | 71 | 49.458 | −0.127 | −12.536 | 1.00 | 18.11 | O |
| ATOM | 435 | N | ASN | A | 72 | 51.501 | 0.474 | −13.310 | 1.00 | 19.82 | N |
| ATOM | 436 | CA | ASN | A | 72 | 51.804 | −0.805 | −13.935 | 1.00 | 22.45 | C |
| ATOM | 437 | CB | ASN | A | 72 | 52.260 | −0.591 | −15.384 | 1.00 | 22.22 | C |
| ATOM | 438 | CG | ASN | A | 72 | 52.398 | −1.888 | −16.162 | 1.00 | 23.89 | C |
| ATOM | 439 | OD1 | ASN | A | 72 | 52.535 | −2.981 | −15.592 | 1.00 | 24.57 | O |
| ATOM | 440 | ND2 | ASN | A | 72 | 52.374 | −1.770 | −17.478 | 1.00 | 27.34 | N |
| ATOM | 441 | C | ASN | A | 72 | 52.879 | −1.527 | −13.137 | 1.00 | 23.57 | C |
| ATOM | 442 | O | ASN | A | 72 | 54.059 | −1.196 | −13.222 | 1.00 | 24.44 | O |
| ATOM | 443 | N | LEU | A | 73 | 52.467 | −2.522 | −12.369 | 1.00 | 25.37 | N |
| ATOM | 444 | CA | LEU | A | 73 | 53.373 | −3.164 | −11.438 | 1.00 | 28.05 | C |
| ATOM | 445 | CB | LEU | A | 73 | 52.605 | −4.032 | −10.458 | 1.00 | 27.62 | C |
| ATOM | 446 | CG | LEU | A | 73 | 52.048 | −3.540 | −9.114 | 1.00 | 27.84 | C |
| ATOM | 447 | CD1 | LEU | A | 73 | 51.984 | −2.027 | −8.907 | 1.00 | 26.66 | C |
| ATOM | 448 | CD2 | LEU | A | 73 | 50.736 | −4.137 | −8.993 | 1.00 | 27.12 | C |
| ATOM | 449 | C | LEU | A | 73 | 54.445 | −3.994 | −12.167 | 1.00 | 30.00 | C |
| ATOM | 450 | O | LEU | A | 73 | 55.472 | −4.338 | −11.569 | 1.00 | 30.39 | O |
| ATOM | 451 | N | SER | A | 74 | 54.204 | −4.285 | −13.445 | 1.00 | 32.11 | N |
| ATOM | 452 | CA | SER | A | 74 | 55.160 | −5.020 | −14.288 | 1.00 | 35.10 | C |
| ATOM | 453 | CB | SER | A | 74 | 54.455 | −5.561 | −15.526 | 1.00 | 34.56 | C |
| ATOM | 454 | OG | SER | A | 74 | 53.918 | −6.819 | −15.180 | 1.00 | 38.68 | O |
| ATOM | 455 | C | SER | A | 74 | 56.385 | −4.215 | −14.730 | 1.00 | 36.36 | C |
| ATOM | 456 | O | SER | A | 74 | 57.446 | −4.807 | −14.997 | 1.00 | 37.47 | O |
| ATOM | 457 | N | ARG | A | 75 | 56.219 | −2.892 | −14.845 | 1.00 | 37.14 | N |
| ATOM | 458 | CA | ARG | A | 75 | 57.275 | −1.965 | −15.305 | 1.00 | 38.37 | C |
| ATOM | 459 | CB | ARG | A | 75 | 56.663 | −0.878 | −16.186 | 1.00 | 38.73 | C |
| ATOM | 460 | CG | ARG | A | 75 | 56.111 | −1.333 | −17.531 | 1.00 | 41.49 | C |
| ATOM | 461 | CD | ARG | A | 75 | 56.158 | −0.219 | −18.574 | 1.00 | 48.71 | C |
| ATOM | 462 | NE | ARG | A | 75 | 55.070 | −0.278 | −19.556 | 1.00 | 54.15 | N |
| ATOM | 463 | CZ | ARG | A | 75 | 55.090 | −1.013 | −20.669 | 1.00 | 57.83 | C |
| ATOM | 464 | NH1 | ARG | A | 75 | 56.141 | −1.776 | −20.955 | 1.00 | 59.77 | N |
| ATOM | 465 | NH2 | ARG | A | 75 | 54.054 | −0.994 | −21.500 | 1.00 | 58.78 | N |
| ATOM | 466 | C | ARG | A | 75 | 57.979 | −1.262 | −14.133 | 1.00 | 38.32 | C |
| ATOM | 467 | O | ARG | A | 75 | 57.373 | −1.060 | −13.085 | 1.00 | 38.49 | O |
| ATOM | 468 | N | ARG | A | 76 | 59.251 | −0.894 | −14.304 | 1.00 | 38.37 | N |
| ATOM | 469 | CA | ARG | A | 76 | 59.909 | 0.036 | −13.381 | 1.00 | 38.06 | C |
| ATOM | 470 | CB | ARG | A | 76 | 61.436 | 0.105 | −13.614 | 1.00 | 39.53 | C |
| ATOM | 471 | CG | ARG | A | 76 | 62.299 | −0.398 | −12.428 | 1.00 | 43.41 | C |
| ATOM | 472 | CD | ARG | A | 76 | 63.617 | −1.119 | −12.823 | 1.00 | 50.17 | C |
| ATOM | 473 | NE | ARG | A | 76 | 64.346 | −0.463 | −13.926 | 1.00 | 54.40 | N |
| ATOM | 474 | CZ | ARG | A | 76 | 64.596 | −1.022 | −15.123 | 1.00 | 57.84 | C |
| ATOM | 475 | NH1 | ARG | A | 76 | 64.189 | −2.261 | −15.406 | 1.00 | 58.47 | N |
| ATOM | 476 | NH2 | ARG | A | 76 | 65.264 | −0.339 | −16.048 | 1.00 | 58.85 | N |
| ATOM | 477 | C | ARG | A | 76 | 59.279 | 1.399 | −13.675 | 1.00 | 36.60 | C |
| ATOM | 478 | O | ARG | A | 76 | 59.142 | 1.788 | −14.845 | 1.00 | 37.06 | O |
| ATOM | 479 | N | GLU | A | 77 | 58.859 | 2.106 | −12.633 | 1.00 | 33.97 | N |
| ATOM | 480 | CA | GLU | A | 77 | 58.154 | 3.366 | −12.827 | 1.00 | 31.87 | C |
| ATOM | 481 | CB | GLU | A | 77 | 56.666 | 3.231 | −12.463 | 1.00 | 30.95 | C |
| ATOM | 482 | CG | GLU | A | 77 | 55.853 | 2.289 | −13.355 | 1.00 | 27.89 | C |
| ATOM | 483 | CD | GLU | A | 77 | 54.402 | 2.142 | −12.880 | 1.00 | 23.13 | C |
| ATOM | 484 | OE1 | GLU | A | 77 | 53.476 | 2.291 | −13.701 | 1.00 | 20.65 | O |
| ATOM | 485 | OE2 | GLU | A | 77 | 54.199 | 1.927 | −11.669 | 1.00 | 21.15 | O |
| ATOM | 486 | C | GLU | A | 77 | 58.827 | 4.450 | −11.963 | 1.00 | 31.75 | C |
| ATOM | 487 | O | GLU | A | 77 | 58.800 | 4.367 | −10.722 | 1.00 | 30.76 | O |
| ATOM | 488 | N | PRO | A | 78 | 59.453 | 5.433 | −12.628 | 1.00 | 31.62 | N |
| ATOM | 489 | CA | PRO | A | 78 | 59.955 | 6.659 | −11.975 | 1.00 | 31.13 | C |
| ATOM | 490 | CB | PRO | A | 78 | 60.318 | 7.559 | −13.171 | 1.00 | 32.04 | C |
| ATOM | 491 | CG | PRO | A | 78 | 60.751 | 6.613 | −14.206 | 1.00 | 32.16 | C |
| ATOM | 492 | CD | PRO | A | 78 | 59.766 | 5.433 | −14.074 | 1.00 | 32.01 | C |
| ATOM | 493 | C | PRO | A | 78 | 58.963 | 7.389 | −11.080 | 1.00 | 29.56 | C |
| ATOM | 494 | O | PRO | A | 78 | 59.411 | 7.988 | −10.108 | 1.00 | 29.71 | O |
| ATOM | 495 | N | THR | A | 79 | 57.661 | 7.334 | −11.384 | 1.00 | 28.26 | N |
| ATOM | 496 | CA | THR | A | 79 | 56.633 | 8.028 | −10.585 | 1.00 | 26.58 | C |
| ATOM | 497 | CB | THR | A | 79 | 55.271 | 8.056 | −11.319 | 1.00 | 27.24 | C |
| ATOM | 498 | OG1 | THR | A | 79 | 54.870 | 6.723 | −11.656 | 1.00 | 25.55 | O |
| ATOM | 499 | CG2 | THR | A | 79 | 55.370 | 8.757 | −12.698 | 1.00 | 27.14 | C |
| ATOM | 500 | C | THR | A | 79 | 56.429 | 7.453 | −9.180 | 1.00 | 25.93 | C |
| ATOM | 501 | O | THR | A | 79 | 55.718 | 8.055 | −8.371 | 1.00 | 25.66 | O |
| ATOM | 502 | N | ARG | A | 80 | 57.040 | 6.301 | −8.883 | 1.00 | 24.04 | N |
| ATOM | 503 | CA | ARG | A | 80 | 56.814 | 5.622 | −7.606 | 1.00 | 22.49 | C |
| ATOM | 504 | CB | ARG | A | 80 | 57.177 | 4.150 | −7.719 | 1.00 | 23.43 | C |
| ATOM | 505 | CG | ARG | A | 80 | 56.183 | 3.284 | −8.452 | 1.00 | 23.19 | C |
| ATOM | 506 | CD | ARG | A | 80 | 56.656 | 1.840 | −8.485 | 1.00 | 28.64 | C |
| ATOM | 507 | NE | ARG | A | 80 | 56.139 | 1.137 | −9.637 | 1.00 | 31.48 | N |
| ATOM | 508 | CZ | ARG | A | 80 | 56.130 | −0.170 | −9.776 | 1.00 | 34.38 | C |
| ATOM | 509 | NH1 | ARG | A | 80 | 56.613 | −0.958 | −8.811 | 1.00 | 34.96 | N |
| ATOM | 510 | NH2 | ARG | A | 80 | 55.618 | −0.695 | −10.887 | 1.00 | 33.85 | N |
| ATOM | 511 | C | ARG | A | 80 | 57.635 | 6.169 | −6.467 | 1.00 | 21.79 | C |
| ATOM | 512 | O | ARG | A | 80 | 58.791 | 6.572 | −6.673 | 1.00 | 21.22 | O |
| ATOM | 513 | N | GLN | A | 81 | 57.053 | 6.129 | −5.263 | 1.00 | 19.61 | N |
| ATOM | 514 | CA | GLN | A | 81 | 57.778 | 6.423 | −4.024 | 1.00 | 18.64 | C |
| ATOM | 515 | CB | GLN | A | 81 | 57.245 | 7.700 | −3.381 | 1.00 | 17.73 | C |
| ATOM | 516 | CG | GLN | A | 81 | 57.502 | 9.001 | −4.190 | 1.00 | 16.05 | C |
| ATOM | 517 | CD | GLN | A | 81 | 56.713 | 10.152 | −3.618 | 1.00 | 14.71 | C |
| ATOM | 518 | OE1 | GLN | A | 81 | 56.704 | 10.359 | −2.400 | 1.00 | 20.02 | O |
| ATOM | 519 | NE2 | GLN | A | 81 | 56.007 | 10.869 | −4.464 | 1.00 | 14.23 | N |
| ATOM | 520 | C | GLN | A | 81 | 57.562 | 5.251 | −3.076 | 1.00 | 18.95 | C |
| ATOM | 521 | O | GLN | A | 81 | 56.404 | 4.826 | −2.863 | 1.00 | 17.09 | O |
| ATOM | 522 | N | VAL | A | 82 | 58.655 | 4.749 | −2.496 | 1.00 | 18.86 | N |
| ATOM | 523 | CA | VAL | A | 82 | 58.600 | 3.544 | −1.674 | 1.00 | 19.71 | C |
| ATOM | 524 | CB | VAL | A | 82 | 59.501 | 2.414 | −2.199 | 1.00 | 20.03 | C |
| ATOM | 525 | CG1 | VAL | A | 82 | 59.267 | 1.110 | −1.376 | 1.00 | 20.16 | C |
| ATOM | 526 | CG2 | VAL | A | 82 | 59.267 | 2.178 | −3.704 | 1.00 | 20.05 | C |
| ATOM | 527 | C | VAL | A | 82 | 58.956 | 3.829 | −0.229 | 1.00 | 20.70 | C |
| ATOM | 528 | O | VAL | A | 82 | 60.016 | 4.406 | 0.063 | 1.00 | 20.99 | O |
| ATOM | 529 | N | PHE | A | 83 | 58.065 | 3.422 | 0.670 | 1.00 | 20.61 | N |
| ATOM | 530 | CA | PHE | A | 83 | 58.293 | 3.573 | 2.100 | 1.00 | 20.74 | C |
| ATOM | 531 | CB | PHE | A | 83 | 57.316 | 4.580 | 2.688 | 1.00 | 20.27 | C |
| ATOM | 532 | CG | PHE | A | 83 | 57.497 | 5.969 | 2.120 | 1.00 | 22.34 | C |
| ATOM | 533 | CD1 | PHE | A | 83 | 56.884 | 6.329 | 0.898 | 1.00 | 18.78 | C |
| ATOM | 534 | CE1 | PHE | A | 83 | 57.087 | 7.577 | 0.330 | 1.00 | 21.61 | C |
| ATOM | 535 | CZ | PHE | A | 83 | 57.894 | 8.517 | 0.983 | 1.00 | 22.54 | C |
| ATOM | 536 | CE2 | PHE | A | 83 | 58.530 | 8.167 | 2.201 | 1.00 | 25.24 | C |
| ATOM | 537 | CD2 | PHE | A | 83 | 58.320 | 6.877 | 2.763 | 1.00 | 22.42 | C |
| ATOM | 538 | C | PHE | A | 83 | 58.224 | 2.267 | 2.825 | 1.00 | 21.61 | C |
| ATOM | 539 | O | PHE | A | 83 | 57.772 | 1.263 | 2.245 | 1.00 | 22.41 | O |
| ATOM | 540 | N | ALA | A | 84 | 58.668 | 2.286 | 4.086 | 1.00 | 22.15 | N |
| ATOM | 541 | CA | ALA | A | 84 | 58.565 | 1.147 | 4.998 | 1.00 | 22.80 | C |
| ATOM | 542 | CB | ALA | A | 84 | 59.937 | 0.824 | 5.636 | 1.00 | 22.60 | C |
| ATOM | 543 | C | ALA | A | 84 | 57.576 | 1.499 | 6.074 | 1.00 | 22.65 | C |
| ATOM | 544 | O | ALA | A | 84 | 57.187 | 2.660 | 6.207 | 1.00 | 23.15 | O |
| ATOM | 545 | N | VAL | A | 85 | 57.157 | 0.512 | 6.855 | 1.00 | 22.71 | N |
| ATOM | 546 | CA | VAL | A | 85 | 56.184 | 0.770 | 7.896 | 1.00 | 22.96 | C |
| ATOM | 547 | CB | VAL | A | 85 | 55.133 | −0.373 | 8.026 | 1.00 | 23.12 | C |
| ATOM | 548 | CG1 | VAL | A | 85 | 54.193 | −0.115 | 9.226 | 1.00 | 22.14 | C |
| ATOM | 549 | CG2 | VAL | A | 85 | 54.347 | −0.591 | 6.703 | 1.00 | 22.58 | C |
| ATOM | 550 | C | VAL | A | 85 | 56.887 | 0.989 | 9.229 | 1.00 | 24.55 | C |
| ATOM | 551 | O | VAL | A | 85 | 57.675 | 0.161 | 9.686 | 1.00 | 23.99 | O |
| ATOM | 552 | N | GLN | A | 86 | 56.568 | 2.105 | 9.853 | 1.00 | 25.62 | N |
| ATOM | 553 | CA | GLN | A | 86 | 57.234 | 2.525 | 11.078 | 1.00 | 27.28 | C |
| ATOM | 554 | CB | GLN | A | 86 | 57.282 | 4.056 | 11.136 | 1.00 | 28.04 | C |
| ATOM | 555 | CG | GLN | A | 86 | 57.933 | 4.678 | 12.377 | 1.00 | 30.26 | C |
| ATOM | 556 | CD | GLN | A | 86 | 58.369 | 6.100 | 12.095 | 1.00 | 34.76 | C |
| ATOM | 557 | OE1 | GLN | A | 86 | 57.663 | 7.051 | 12.427 | 1.00 | 37.12 | O |
| ATOM | 558 | NE2 | GLN | A | 86 | 59.523 | 6.248 | 11.442 | 1.00 | 37.57 | N |
| ATOM | 559 | C | GLN | A | 86 | 56.443 | 1.993 | 12.236 | 1.00 | 27.53 | C |
| ATOM | 560 | O | GLN | A | 86 | 57.017 | 1.445 | 13.181 | 1.00 | 28.15 | O |
| ATOM | 561 | N | ARG | A | 87 | 55.118 | 2.129 | 12.145 | 1.00 | 27.47 | N |
| ATOM | 562 | CA | ARG | A | 87 | 54.229 | 1.848 | 13.258 | 1.00 | 27.55 | C |
| ATOM | 563 | CB | ARG | A | 87 | 54.107 | 3.129 | 14.127 | 1.00 | 27.80 | C |
| ATOM | 564 | CG | ARG | A | 87 | 53.357 | 3.007 | 15.477 | 1.00 | 30.24 | C |
| ATOM | 565 | CD | ARG | A | 87 | 53.577 | 4.183 | 16.462 | 1.00 | 35.34 | C |
| ATOM | 566 | NE | ARG | A | 87 | 52.922 | 3.941 | 17.763 | 1.00 | 41.08 | N |
| ATOM | 567 | CZ | ARG | A | 87 | 52.428 | 4.887 | 18.590 | 1.00 | 42.93 | C |
| ATOM | 568 | NH1 | ARG | A | 87 | 52.514 | 6.184 | 18.287 | 1.00 | 41.31 | N |
| ATOM | 569 | NH2 | ARG | A | 87 | 51.837 | 4.524 | 19.737 | 1.00 | 43.69 | N |
| ATOM | 570 | C | ARG | A | 87 | 52.884 | 1.382 | 12.672 | 1.00 | 27.27 | C |
| ATOM | 571 | O | ARG | A | 87 | 52.480 | 1.811 | 11.593 | 1.00 | 26.30 | O |
| ATOM | 572 | N | ILE | A | 88 | 52.214 | 0.492 | 13.375 | 1.00 | 26.94 | N |
| ATOM | 573 | CA | ILE | A | 88 | 50.893 | 0.028 | 12.989 | 1.00 | 26.91 | C |
| ATOM | 574 | CB | ILE | A | 88 | 50.912 | −1.504 | 12.917 | 1.00 | 27.43 | C |
| ATOM | 575 | CG1 | ILE | A | 88 | 51.573 | −1.953 | 11.640 | 1.00 | 29.79 | C |
| ATOM | 576 | CD1 | ILE | A | 88 | 51.810 | −3.431 | 11.665 | 1.00 | 34.57 | C |
| ATOM | 577 | CG2 | ILE | A | 88 | 49.502 | −2.118 | 13.036 | 1.00 | 28.18 | C |
| ATOM | 578 | C | ILE | A | 88 | 49.878 | 0.468 | 14.023 | 1.00 | 25.62 | C |
| ATOM | 579 | O | ILE | A | 88 | 50.113 | 0.327 | 15.228 | 1.00 | 26.39 | O |
| ATOM | 580 | N | PHE | A | 89 | 48.732 | 0.963 | 13.570 | 1.00 | 24.10 | N |
| ATOM | 581 | CA | PHE | A | 89 | 47.620 | 1.232 | 14.475 | 1.00 | 23.18 | C |
| ATOM | 582 | CB | PHE | A | 89 | 47.170 | 2.689 | 14.394 | 1.00 | 23.38 | C |
| ATOM | 583 | CG | PHE | A | 89 | 48.232 | 3.666 | 14.745 | 1.00 | 25.13 | C |
| ATOM | 584 | CD1 | PHE | A | 89 | 48.239 | 4.264 | 16.005 | 1.00 | 26.10 | C |
| ATOM | 585 | CE1 | PHE | A | 89 | 49.218 | 5.191 | 16.333 | 1.00 | 28.94 | C |
| ATOM | 586 | CZ | PHE | A | 89 | 50.209 | 5.514 | 15.406 | 1.00 | 26.34 | C |
| ATOM | 587 | CE2 | PHE | A | 89 | 50.224 | 4.919 | 14.155 | 1.00 | 26.73 | C |
| ATOM | 588 | CD2 | PHE | A | 89 | 49.231 | 3.991 | 13.824 | 1.00 | 25.58 | C |
| ATOM | 589 | C | PHE | A | 89 | 46.429 | 0.353 | 14.126 | 1.00 | 22.56 | C |
| ATOM | 590 | O | PHE | A | 89 | 45.971 | 0.355 | 12.984 | 1.00 | 20.62 | O |
| ATOM | 591 | N | GLU | A | 90 | 45.890 | −0.343 | 15.115 | 1.00 | 22.12 | N |
| ATOM | 592 | CA | GLU | A | 90 | 44.711 | −1.180 | 14.853 | 1.00 | 22.79 | C |
| ATOM | 593 | CB | GLU | A | 90 | 44.997 | −2.673 | 15.077 | 1.00 | 23.76 | C |
| ATOM | 594 | CG | GLU | A | 90 | 45.399 | −3.320 | 13.739 | 1.00 | 30.50 | C |
| ATOM | 595 | CD | GLU | A | 90 | 45.211 | −4.834 | 13.665 | 1.00 | 36.47 | C |
| ATOM | 596 | OE1 | GLU | A | 90 | 44.302 | −5.379 | 14.307 | 1.00 | 40.00 | O |
| ATOM | 597 | OE2 | GLU | A | 90 | 45.990 | −5.483 | 12.940 | 1.00 | 40.44 | O |
| ATOM | 598 | C | GLU | A | 90 | 43.550 | −0.701 | 15.657 | 1.00 | 21.58 | C |
| ATOM | 599 | O | GLU | A | 90 | 43.717 | 0.140 | 16.529 | 1.00 | 21.39 | O |
| ATOM | 600 | N | ASP | A | 91 | 42.376 | −1.238 | 15.361 | 1.00 | 19.49 | N |
| ATOM | 601 | CA | ASP | A | 91 | 41.150 | −0.761 | 15.977 | 1.00 | 19.60 | C |
| ATOM | 602 | CB | ASP | A | 91 | 40.506 | 0.328 | 15.108 | 1.00 | 17.87 | C |
| ATOM | 603 | CG | ASP | A | 91 | 39.439 | 1.121 | 15.850 | 1.00 | 19.77 | C |
| ATOM | 604 | OD1 | ASP | A | 91 | 38.633 | 1.804 | 15.155 | 1.00 | 22.51 | O |
| ATOM | 605 | OD2 | ASP | A | 91 | 39.300 | 1.105 | 17.110 | 1.00 | 16.70 | O |
| ATOM | 606 | C | ASP | A | 91 | 40.158 | −1.890 | 16.217 | 1.00 | 18.93 | C |
| ATOM | 607 | O | ASP | A | 91 | 38.986 | −1.807 | 15.822 | 1.00 | 18.22 | O |
| ATOM | 608 | N | GLY | A | 92 | 40.635 | −2.951 | 16.850 | 1.00 | 19.13 | N |
| ATOM | 609 | CA | GLY | A | 92 | 39.731 | −4.005 | 17.290 | 1.00 | 19.43 | C |
| ATOM | 610 | C | GLY | A | 92 | 39.401 | −5.057 | 16.250 | 1.00 | 20.01 | C |
| ATOM | 611 | O | GLY | A | 92 | 38.360 | −5.706 | 16.369 | 1.00 | 20.82 | O |
| ATOM | 612 | N | TYR | A | 94 | 40.277 | −5.232 | 15.255 | 1.00 | 19.85 | N |
| ATOM | 613 | CA | TYR | A | 94 | 40.173 | −6.301 | 14.242 | 1.00 | 19.64 | C |
| ATOM | 614 | CB | TYR | A | 94 | 41.577 | −6.578 | 13.665 | 1.00 | 20.15 | C |
| ATOM | 615 | CG | TYR | A | 94 | 41.617 | −7.661 | 12.617 | 1.00 | 19.00 | C |
| ATOM | 616 | CD1 | TYR | A | 94 | 42.655 | −8.597 | 12.593 | 1.00 | 17.83 | C |
| ATOM | 617 | CE1 | TYR | A | 94 | 42.672 | −9.607 | 11.622 | 1.00 | 19.09 | C |
| ATOM | 618 | CZ | TYR | A | 94 | 41.674 | −9.683 | 10.705 | 1.00 | 18.24 | C |
| ATOM | 619 | OH | TYR | A | 94 | 41.678 | −10.685 | 9.752 | 1.00 | 24.02 | O |
| ATOM | 620 | CE2 | TYR | A | 94 | 40.638 | −8.770 | 10.706 | 1.00 | 21.68 | C |
| ATOM | 621 | CD2 | TYR | A | 94 | 40.610 | −7.763 | 11.662 | 1.00 | 16.89 | C |
| ATOM | 622 | C | TYR | A | 94 | 39.606 | −7.604 | 14.800 | 1.00 | 20.11 | C |
| ATOM | 623 | O | TYR | A | 94 | 40.191 | −8.210 | 15.685 | 1.00 | 21.16 | O |
| ATOM | 624 | N | ASP | A | 95 | 38.469 | −8.040 | 14.300 | 1.00 | 21.06 | N |
| ATOM | 625 | CA | ASP | A | 95 | 37.917 | −9.299 | 14.768 | 1.00 | 22.48 | C |
| ATOM | 626 | CB | ASP | A | 95 | 36.593 | −9.039 | 15.477 | 1.00 | 22.88 | C |
| ATOM | 627 | CG | ASP | A | 95 | 35.987 | −10.297 | 16.066 | 1.00 | 26.14 | C |
| ATOM | 628 | OD1 | ASP | A | 95 | 36.495 | −11.421 | 15.805 | 1.00 | 29.07 | O |
| ATOM | 629 | OD2 | ASP | A | 95 | 34.994 | −10.254 | 16.797 | 1.00 | 26.58 | O |
| ATOM | 630 | C | ASP | A | 95 | 37.700 | −10.246 | 13.593 | 1.00 | 22.16 | C |
| ATOM | 631 | O | ASP | A | 95 | 36.631 | −10.237 | 13.012 | 1.00 | 22.59 | O |
| ATOM | 632 | N | PRO | A | 98 | 38.701 | −11.058 | 13.243 | 1.00 | 22.67 | N |
| ATOM | 633 | CA | PRO | A | 98 | 38.574 | −11.942 | 12.090 | 1.00 | 23.41 | C |
| ATOM | 634 | CB | PRO | A | 98 | 39.938 | −12.651 | 11.992 | 1.00 | 23.44 | C |
| ATOM | 635 | CG | PRO | A | 98 | 40.743 | −12.300 | 13.194 | 1.00 | 23.51 | C |
| ATOM | 636 | CD | PRO | A | 98 | 40.003 | −11.196 | 13.930 | 1.00 | 23.23 | C |
| ATOM | 637 | C | PRO | A | 98 | 37.419 | −12.966 | 12.237 | 1.00 | 24.91 | C |
| ATOM | 638 | O | PRO | A | 98 | 36.850 | −13.328 | 11.208 | 1.00 | 25.39 | O |
| ATOM | 639 | N | VAL | A | 99 | 37.046 | −13.425 | 13.435 | 1.00 | 25.08 | N |
| ATOM | 640 | CA | VAL | A | 99 | 35.975 | −14.441 | 13.435 | 1.00 | 25.78 | C |
| ATOM | 641 | CB | VAL | A | 99 | 35.986 | −15.469 | 14.620 | 1.00 | 26.23 | C |
| ATOM | 642 | CG1 | VAL | A | 99 | 37.290 | −15.404 | 15.452 | 1.00 | 29.20 | C |
| ATOM | 643 | CG2 | VAL | A | 99 | 34.722 | −15.353 | 15.488 | 1.00 | 28.49 | C |
| ATOM | 644 | C | VAL | A | 99 | 34.619 | −13.847 | 13.160 | 1.00 | 24.71 | C |
| ATOM | 645 | O | VAL | A | 99 | 33.799 | −14.457 | 12.473 | 1.00 | 25.63 | O |
| ATOM | 646 | N | ASN | A | 99A | 34.386 | −12.629 | 13.632 | 1.00 | 24.37 | N |
| ATOM | 647 | CA | ASN | A | 99A | 33.134 | −11.954 | 13.312 | 1.00 | 24.34 | C |
| ATOM | 648 | CB | ASN | A | 99A | 32.621 | −11.170 | 14.537 | 1.00 | 24.61 | C |
| ATOM | 649 | CG | ASN | A | 99A | 32.159 | −12.095 | 15.658 | 1.00 | 26.37 | C |
| ATOM | 650 | OD1 | ASN | A | 99A | 31.191 | −12.844 | 15.486 | 1.00 | 27.11 | O |
| ATOM | 651 | ND2 | ASN | A | 99A | 32.871 | −12.076 | 16.787 | 1.00 | 23.57 | N |
| ATOM | 652 | C | ASN | A | 99A | 33.225 | −11.074 | 12.054 | 1.00 | 23.78 | C |
| ATOM | 653 | O | ASN | A | 99A | 32.243 | −10.469 | 11.652 | 1.00 | 24.46 | O |
| ATOM | 654 | N | LEU | A | 99B | 34.407 | −11.030 | 11.448 | 1.00 | 23.58 | N |
| ATOM | 655 | CA | LEU | A | 99B | 34.702 | −10.192 | 10.269 | 1.00 | 24.01 | C |
| ATOM | 656 | CB | LEU | A | 99B | 33.922 | −10.656 | 9.039 | 1.00 | 25.02 | C |
| ATOM | 657 | CG | LEU | A | 99B | 34.206 | −11.969 | 8.285 | 1.00 | 27.16 | C |
| ATOM | 658 | CD1 | LEU | A | 99B | 35.441 | −12.725 | 8.716 | 1.00 | 29.00 | C |
| ATOM | 659 | CD2 | LEU | A | 99B | 32.963 | −12.822 | 8.377 | 1.00 | 30.53 | C |
| ATOM | 660 | C | LEU | A | 99B | 34.450 | −8.695 | 10.512 | 1.00 | 22.76 | C |
| ATOM | 661 | O | LEU | A | 99B | 33.908 | −7.995 | 9.652 | 1.00 | 22.96 | O |
| ATOM | 662 | N | LEU | A | 100 | 34.872 | −8.211 | 11.676 | 1.00 | 21.20 | N |
| ATOM | 663 | CA | LEU | A | 100 | 34.606 | −6.817 | 12.060 | 1.00 | 19.43 | C |
| ATOM | 664 | CB | LEU | A | 100 | 33.863 | −6.774 | 13.400 | 1.00 | 19.55 | C |
| ATOM | 665 | CG | LEU | A | 100 | 32.533 | −7.517 | 13.518 | 1.00 | 21.11 | C |
| ATOM | 666 | CD1 | LEU | A | 100 | 32.105 | −7.573 | 14.991 | 1.00 | 21.45 | C |
| ATOM | 667 | CD2 | LEU | A | 100 | 31.471 | −6.817 | 12.695 | 1.00 | 24.39 | C |
| ATOM | 668 | C | LEU | A | 100 | 35.869 | −5.995 | 12.190 | 1.00 | 17.10 | C |
| ATOM | 669 | O | LEU | A | 100 | 36.917 | −6.520 | 12.552 | 1.00 | 16.85 | O |
| ATOM | 670 | N | ASN | A | 101 | 35.740 | −4.686 | 11.976 | 1.00 | 16.10 | N |
| ATOM | 671 | CA | ASN | A | 101 | 36.840 | −3.732 | 12.224 | 1.00 | 16.08 | C |
| ATOM | 672 | CB | ASN | A | 101 | 37.238 | −3.620 | 13.717 | 1.00 | 15.31 | C |
| ATOM | 673 | CG | ASN | A | 101 | 36.044 | −3.379 | 14.638 | 1.00 | 16.86 | C |
| ATOM | 674 | OD1 | ASN | A | 101 | 35.968 | −3.958 | 15.750 | 1.00 | 20.31 | O |
| ATOM | 675 | ND2 | ASN | A | 101 | 35.109 | −2.542 | 14.204 | 1.00 | 11.07 | N |
| ATOM | 676 | C | ASN | A | 101 | 38.073 | −4.040 | 11.378 | 1.00 | 15.37 | C |
| ATOM | 677 | O | ASN | A | 101 | 39.190 | −4.043 | 11.854 | 1.00 | 15.25 | O |
| ATOM | 678 | N | ASP | A | 102 | 37.846 | −4.292 | 10.105 | 1.00 | 15.47 | N |
| ATOM | 679 | CA | ASP | A | 102 | 38.924 | −4.712 | 9.234 | 1.00 | 14.69 | C |
| ATOM | 680 | CB | ASP | A | 102 | 38.364 | −5.590 | 8.141 | 1.00 | 15.21 | C |
| ATOM | 681 | CG | ASP | A | 102 | 39.428 | −6.348 | 7.402 | 1.00 | 14.53 | C |
| ATOM | 682 | OD1 | ASP | A | 102 | 40.600 | −6.333 | 7.845 | 1.00 | 15.24 | O |
| ATOM | 683 | OD2 | ASP | A | 102 | 39.160 | −7.003 | 6.366 | 1.00 | 14.85 | O |
| ATOM | 684 | C | ASP | A | 102 | 39.629 | −3.486 | 8.656 | 1.00 | 15.07 | C |
| ATOM | 685 | O | ASP | A | 102 | 39.562 | −3.196 | 7.448 | 1.00 | 15.03 | O |
| ATOM | 686 | N | ILE | A | 103 | 40.363 | −2.808 | 9.524 | 1.00 | 15.41 | N |
| ATOM | 687 | CA | ILE | A | 103 | 41.056 | −1.590 | 9.153 | 1.00 | 15.54 | C |
| ATOM | 688 | CB | ILE | A | 103 | 40.137 | −0.365 | 9.430 | 1.00 | 15.33 | C |
| ATOM | 689 | CG1 | ILE | A | 103 | 40.817 | 0.951 | 9.014 | 1.00 | 14.39 | C |
| ATOM | 690 | CD1 | ILE | A | 103 | 39.787 | 2.145 | 8.917 | 1.00 | 14.92 | C |
| ATOM | 691 | CG2 | ILE | A | 103 | 39.671 | −0.363 | 10.944 | 1.00 | 16.71 | C |
| ATOM | 692 | C | ILE | A | 103 | 42.319 | −1.531 | 9.995 | 1.00 | 16.53 | C |
| ATOM | 693 | O | ILE | A | 103 | 42.316 | −1.907 | 11.182 | 1.00 | 15.58 | O |
| ATOM | 694 | N | VAL | A | 104 | 43.392 | −1.036 | 9.370 | 1.00 | 16.42 | N |
| ATOM | 695 | CA | VAL | A | 104 | 44.645 | −0.793 | 10.042 | 1.00 | 16.86 | C |
| ATOM | 696 | CB | VAL | A | 104 | 45.610 | −2.012 | 9.826 | 1.00 | 16.96 | C |
| ATOM | 697 | CG1 | VAL | A | 104 | 45.926 | −2.237 | 8.339 | 1.00 | 16.13 | C |
| ATOM | 698 | CG2 | VAL | A | 104 | 46.868 | −1.842 | 10.615 | 1.00 | 20.64 | C |
| ATOM | 699 | C | VAL | A | 104 | 45.217 | 0.479 | 9.453 | 1.00 | 16.70 | C |
| ATOM | 700 | O | VAL | A | 104 | 45.035 | 0.748 | 8.258 | 1.00 | 16.20 | O |
| ATOM | 701 | N | ILE | A | 105 | 45.901 | 1.280 | 10.265 | 1.00 | 16.93 | N |
| ATOM | 702 | CA | ILE | A | 105 | 46.673 | 2.388 | 9.712 | 1.00 | 16.24 | C |
| ATOM | 703 | CB | ILE | A | 105 | 46.296 | 3.735 | 10.410 | 1.00 | 16.24 | C |
| ATOM | 704 | CG1 | ILE | A | 105 | 44.876 | 4.156 | 10.032 | 1.00 | 15.46 | C |
| ATOM | 705 | CD1 | ILE | A | 105 | 44.415 | 5.448 | 10.729 | 1.00 | 15.30 | C |
| ATOM | 706 | CG2 | ILE | A | 105 | 47.346 | 4.831 | 10.102 | 1.00 | 13.02 | C |
| ATOM | 707 | C | ILE | A | 105 | 48.140 | 2.075 | 9.867 | 1.00 | 17.68 | C |
| ATOM | 708 | O | ILE | A | 105 | 48.586 | 1.629 | 10.946 | 1.00 | 18.71 | O |
| ATOM | 709 | N | LEU | A | 106 | 48.893 | 2.259 | 8.785 | 1.00 | 17.88 | N |
| ATOM | 710 | CA | LEU | A | 106 | 50.340 | 2.067 | 8.787 | 1.00 | 18.28 | C |
| ATOM | 711 | CB | LEU | A | 106 | 50.834 | 1.153 | 7.650 | 1.00 | 17.33 | C |
| ATOM | 712 | CG | LEU | A | 106 | 49.960 | −0.087 | 7.395 | 1.00 | 18.80 | C |
| ATOM | 713 | CD1 | LEU | A | 106 | 50.294 | −0.786 | 6.070 | 1.00 | 19.70 | C |
| ATOM | 714 | CD2 | LEU | A | 106 | 50.059 | −1.058 | 8.571 | 1.00 | 14.80 | C |
| ATOM | 715 | C | LEU | A | 106 | 51.010 | 3.403 | 8.663 | 1.00 | 19.03 | C |
| ATOM | 716 | O | LEU | A | 106 | 50.876 | 4.111 | 7.637 | 1.00 | 18.46 | O |
| ATOM | 717 | N | GLN | A | 107 | 51.754 | 3.733 | 9.706 | 1.00 | 19.57 | N |
| ATOM | 718 | CA | GLN | A | 107 | 52.610 | 4.925 | 9.703 | 1.00 | 20.59 | C |
| ATOM | 719 | CB | GLN | A | 107 | 52.981 | 5.344 | 11.132 | 1.00 | 20.89 | C |
| ATOM | 720 | CG | GLN | A | 107 | 53.572 | 6.769 | 11.148 | 1.00 | 20.47 | C |
| ATOM | 721 | CD | GLN | A | 107 | 53.857 | 7.348 | 12.516 | 1.00 | 22.91 | C |
| ATOM | 722 | OE1 | GLN | A | 107 | 54.600 | 8.334 | 12.597 | 1.00 | 26.39 | O |
| ATOM | 723 | NE2 | GLN | A | 107 | 53.277 | 6.785 | 13.575 | 1.00 | 22.10 | N |
| ATOM | 724 | C | GLN | A | 107 | 53.870 | 4.676 | 8.899 | 1.00 | 21.30 | C |
| ATOM | 725 | O | GLN | A | 107 | 54.590 | 3.717 | 9.140 | 1.00 | 21.42 | O |
| ATOM | 726 | N | LEU | A | 108 | 54.162 | 5.554 | 7.949 | 1.00 | 21.97 | N |
| ATOM | 727 | CA | LEU | A | 108 | 55.311 | 5.365 | 7.100 | 1.00 | 22.93 | C |
| ATOM | 728 | CB | LEU | A | 108 | 55.078 | 6.094 | 5.774 | 1.00 | 23.16 | C |
| ATOM | 729 | CG | LEU | A | 108 | 54.311 | 5.427 | 4.627 | 1.00 | 24.20 | C |
| ATOM | 730 | CD1 | LEU | A | 108 | 53.604 | 6.444 | 3.737 | 1.00 | 21.00 | C |
| ATOM | 731 | CD2 | LEU | A | 108 | 53.416 | 4.283 | 5.045 | 1.00 | 20.32 | C |
| ATOM | 732 | C | LEU | A | 108 | 56.607 | 5.864 | 7.796 | 1.00 | 24.40 | C |
| ATOM | 733 | O | LEU | A | 108 | 56.546 | 6.596 | 8.796 | 1.00 | 24.82 | O |
| ATOM | 734 | N | ASN | A | 109 | 57.747 | 5.440 | 7.265 | 1.00 | 25.15 | N |
| ATOM | 735 | CA | ASN | A | 109 | 59.074 | 5.825 | 7.764 | 1.00 | 27.55 | C |
| ATOM | 736 | CB | ASN | A | 109 | 60.107 | 4.729 | 7.440 | 1.00 | 27.12 | C |
| ATOM | 737 | CG | ASN | A | 109 | 60.434 | 4.633 | 5.943 | 1.00 | 31.67 | C |
| ATOM | 738 | OD1 | ASN | A | 109 | 59.543 | 4.712 | 5.090 | 1.00 | 31.06 | O |
| ATOM | 739 | ND2 | ASN | A | 109 | 61.724 | 4.420 | 5.628 | 1.00 | 35.21 | N |
| ATOM | 740 | C | ASN | A | 109 | 59.526 | 7.163 | 7.191 | 1.00 | 27.36 | C |
| ATOM | 741 | O | ASN | A | 109 | 60.700 | 7.515 | 7.257 | 1.00 | 29.67 | O |
| ATOM | 742 | N | GLY | A | 110 | 58.594 | 7.913 | 6.625 | 1.00 | 26.54 | N |
| ATOM | 743 | CA | GLY | A | 110 | 58.904 | 9.185 | 6.002 | 1.00 | 25.47 | C |
| ATOM | 744 | C | GLY | A | 110 | 57.585 | 9.746 | 5.516 | 1.00 | 25.29 | C |
| ATOM | 745 | O | GLY | A | 110 | 56.528 | 9.141 | 5.788 | 1.00 | 24.98 | O |
| ATOM | 746 | N | SER | A | 111 | 57.654 | 10.871 | 4.801 | 1.00 | 23.61 | N |
| ATOM | 747 | CA | SER | A | 111 | 56.500 | 11.533 | 4.228 | 1.00 | 23.28 | C |
| ATOM | 748 | CB | SER | A | 111 | 56.380 | 12.972 | 4.716 | 1.00 | 23.35 | C |
| ATOM | 749 | OG | SER | A | 111 | 55.920 | 12.998 | 6.055 | 1.00 | 27.02 | O |
| ATOM | 750 | C | SER | A | 111 | 56.612 | 11.549 | 2.732 | 1.00 | 22.66 | C |
| ATOM | 751 | O | SER | A | 111 | 57.668 | 11.814 | 2.178 | 1.00 | 21.42 | O |
| ATOM | 752 | N | ALA | A | 112 | 55.496 | 11.225 | 2.092 | 1.00 | 21.56 | N |
| ATOM | 753 | CA | ALA | A | 112 | 55.406 | 11.219 | 0.650 | 1.00 | 21.04 | C |
| ATOM | 754 | CB | ALA | A | 112 | 54.033 | 10.678 | 0.229 | 1.00 | 20.66 | C |
| ATOM | 755 | C | ALA | A | 112 | 55.591 | 12.641 | 0.112 | 1.00 | 20.83 | C |
| ATOM | 756 | O | ALA | A | 112 | 55.174 | 13.613 | 0.740 | 1.00 | 19.93 | O |
| ATOM | 757 | N | THR | A | 113 | 56.200 | 12.734 | −1.057 | 1.00 | 19.84 | N |
| ATOM | 758 | CA | THR | A | 113 | 56.219 | 13.974 | −1.830 | 1.00 | 20.52 | C |
| ATOM | 759 | CB | THR | A | 113 | 57.490 | 13.966 | −2.731 | 1.00 | 20.47 | C |
| ATOM | 760 | OG1 | THR | A | 113 | 58.618 | 14.172 | −1.866 | 1.00 | 21.95 | O |
| ATOM | 761 | CG2 | THR | A | 113 | 57.547 | 15.140 | −3.751 | 1.00 | 20.02 | C |
| ATOM | 762 | C | THR | A | 113 | 54.911 | 14.071 | −2.618 | 1.00 | 19.92 | C |
| ATOM | 763 | O | THR | A | 113 | 54.717 | 13.351 | −3.580 | 1.00 | 19.79 | O |
| ATOM | 764 | N | ILE | A | 114 | 54.004 | 14.953 | −2.194 | 1.00 | 20.47 | N |
| ATOM | 765 | CA | ILE | A | 114 | 52.717 | 15.111 | −2.906 | 1.00 | 19.99 | C |
| ATOM | 766 | CB | ILE | A | 114 | 51.685 | 15.900 | −2.052 | 1.00 | 20.77 | C |
| ATOM | 767 | CG1 | ILE | A | 114 | 51.520 | 15.283 | −0.650 | 1.00 | 19.31 | C |
| ATOM | 768 | CD1 | ILE | A | 114 | 51.056 | 13.786 | −0.653 | 1.00 | 20.07 | C |
| ATOM | 769 | CG2 | ILE | A | 114 | 50.318 | 16.067 | −2.844 | 1.00 | 18.89 | C |
| ATOM | 770 | C | ILE | A | 114 | 52.882 | 15.756 | −4.279 | 1.00 | 21.24 | C |
| ATOM | 771 | O | ILE | A | 114 | 53.430 | 16.861 | −4.391 | 1.00 | 20.41 | O |
| ATOM | 772 | N | ASN | A | 115 | 52.433 | 15.057 | −5.320 | 1.00 | 21.93 | N |
| ATOM | 773 | CA | ASN | A | 115 | 52.555 | 15.532 | −6.710 | 1.00 | 22.37 | C |
| ATOM | 774 | CB | ASN | A | 115 | 53.936 | 15.218 | −7.342 | 1.00 | 22.21 | C |
| ATOM | 775 | CG | ASN | A | 115 | 54.298 | 13.729 | −7.360 | 1.00 | 22.87 | C |
| ATOM | 776 | OD1 | ASN | A | 115 | 55.488 | 13.361 | −7.308 | 1.00 | 23.06 | O |
| ATOM | 777 | ND2 | ASN | A | 115 | 53.302 | 12.873 | −7.460 | 1.00 | 19.24 | N |
| ATOM | 778 | C | ASN | A | 115 | 51.362 | 15.082 | −7.563 | 1.00 | 23.42 | C |
| ATOM | 779 | O | ASN | A | 115 | 50.344 | 14.660 | −7.009 | 1.00 | 23.46 | O |
| ATOM | 780 | N | ALA | A | 116 | 51.454 | 15.197 | −8.885 | 1.00 | 23.84 | N |
| ATOM | 781 | CA | ALA | A | 116 | 50.417 | 14.652 | −9.777 | 1.00 | 24.07 | C |
| ATOM | 782 | CB | ALA | A | 116 | 50.879 | 14.751 | −11.246 | 1.00 | 24.02 | C |
| ATOM | 783 | C | ALA | A | 116 | 50.042 | 13.180 | −9.459 | 1.00 | 23.68 | C |
| ATOM | 784 | O | ALA | A | 116 | 48.886 | 12.793 | −9.550 | 1.00 | 23.73 | O |
| ATOM | 785 | N | ASN | A | 117 | 51.033 | 12.383 | −9.081 | 1.00 | 23.03 | N |
| ATOM | 786 | CA | ASN | A | 117 | 50.867 | 10.935 | −9.005 | 1.00 | 22.22 | C |
| ATOM | 787 | CB | ASN | A | 117 | 52.061 | 10.274 | −9.678 | 1.00 | 23.04 | C |
| ATOM | 788 | CG | ASN | A | 117 | 52.180 | 10.663 | −11.158 | 1.00 | 25.92 | C |
| ATOM | 789 | OD1 | ASN | A | 117 | 53.284 | 10.755 | −11.676 | 1.00 | 32.25 | O |
| ATOM | 790 | ND2 | ASN | A | 117 | 51.038 | 10.946 | −11.822 | 1.00 | 25.33 | N |
| ATOM | 791 | C | ASN | A | 117 | 50.640 | 10.362 | −7.603 | 1.00 | 21.25 | C |
| ATOM | 792 | O | ASN | A | 117 | 50.316 | 9.159 | −7.453 | 1.00 | 19.76 | O |
| ATOM | 793 | N | VAL | A | 118 | 50.810 | 11.216 | −6.587 | 1.00 | 19.32 | N |
| ATOM | 794 | CA | VAL | A | 118 | 50.797 | 10.782 | −5.197 | 1.00 | 17.68 | C |
| ATOM | 795 | CB | VAL | A | 118 | 52.259 | 10.584 | −4.648 | 1.00 | 18.48 | C |
| ATOM | 796 | CG1 | VAL | A | 118 | 52.250 | 10.336 | −3.168 | 1.00 | 16.47 | C |
| ATOM | 797 | CG2 | VAL | A | 118 | 52.998 | 9.402 | −5.356 | 1.00 | 17.11 | C |
| ATOM | 798 | C | VAL | A | 118 | 50.013 | 11.836 | −4.424 | 1.00 | 17.72 | C |
| ATOM | 799 | O | VAL | A | 118 | 50.495 | 12.987 | −4.263 | 1.00 | 17.25 | O |
| ATOM | 800 | N | GLN | A | 119 | 48.799 | 11.482 | −3.987 | 1.00 | 15.65 | N |
| ATOM | 801 | CA | GLN | A | 119 | 47.890 | 12.470 | −3.365 | 1.00 | 16.41 | C |
| ATOM | 802 | CB | GLN | A | 119 | 46.848 | 13.013 | −4.378 | 1.00 | 16.22 | C |
| ATOM | 803 | CG | GLN | A | 119 | 47.473 | 13.945 | −5.425 | 1.00 | 21.16 | C |
| ATOM | 804 | CD | GLN | A | 119 | 46.457 | 14.712 | −6.254 | 1.00 | 29.22 | C |
| ATOM | 805 | OE1 | GLN | A | 119 | 46.686 | 14.957 | −7.449 | 1.00 | 36.19 | O |
| ATOM | 806 | NE2 | GLN | A | 119 | 45.345 | 15.119 | −5.635 | 1.00 | 33.12 | N |
| ATOM | 807 | C | GLN | A | 119 | 47.178 | 11.831 | −2.204 | 1.00 | 15.62 | C |
| ATOM | 808 | O | GLN | A | 119 | 46.980 | 10.633 | −2.233 | 1.00 | 13.62 | O |
| ATOM | 809 | N | VAL | A | 120 | 46.806 | 12.606 | −1.183 | 1.00 | 15.46 | N |
| ATOM | 810 | CA | VAL | A | 120 | 45.987 | 12.036 | −0.100 | 1.00 | 16.65 | C |
| ATOM | 811 | CB | VAL | A | 120 | 46.372 | 12.569 | 1.338 | 1.00 | 18.17 | C |
| ATOM | 812 | CG1 | VAL | A | 120 | 47.734 | 13.325 | 1.324 | 1.00 | 16.09 | C |
| ATOM | 813 | CG2 | VAL | A | 120 | 45.274 | 13.356 | 1.999 | 1.00 | 19.12 | C |
| ATOM | 814 | C | VAL | A | 120 | 44.485 | 12.069 | −0.427 | 1.00 | 16.51 | C |
| ATOM | 815 | O | VAL | A | 120 | 43.983 | 13.015 | −1.057 | 1.00 | 15.99 | O |
| ATOM | 816 | N | ALA | A | 121 | 43.805 | 10.998 | −0.042 | 1.00 | 14.96 | N |
| ATOM | 817 | CA | ALA | A | 121 | 42.417 | 10.811 | −0.337 | 1.00 | 14.91 | C |
| ATOM | 818 | CB | ALA | A | 121 | 42.017 | 9.332 | −0.124 | 1.00 | 13.37 | C |
| ATOM | 819 | C | ALA | A | 121 | 41.604 | 11.666 | 0.598 | 1.00 | 15.11 | C |
| ATOM | 820 | O | ALA | A | 121 | 42.082 | 12.133 | 1.628 | 1.00 | 15.01 | O |
| ATOM | 821 | N | GLN | A | 122 | 40.353 | 11.854 | 0.253 | 1.00 | 16.12 | N |
| ATOM | 822 | CA | GLN | A | 122 | 39.472 | 12.598 | 1.123 | 1.00 | 17.17 | C |
| ATOM | 823 | CB | GLN | A | 122 | 38.689 | 13.594 | 0.294 | 1.00 | 18.88 | C |
| ATOM | 824 | CG | GLN | A | 122 | 39.521 | 14.549 | −0.537 | 1.00 | 24.58 | C |
| ATOM | 825 | CD | GLN | A | 122 | 38.645 | 15.321 | −1.509 | 1.00 | 33.94 | C |
| ATOM | 826 | OE1 | GLN | A | 122 | 38.760 | 15.149 | −2.737 | 1.00 | 37.83 | O |
| ATOM | 827 | NE2 | GLN | A | 122 | 37.721 | 16.123 | −0.968 | 1.00 | 32.88 | N |
| ATOM | 828 | C | GLN | A | 122 | 38.537 | 11.570 | 1.694 | 1.00 | 17.69 | C |
| ATOM | 829 | O | GLN | A | 122 | 38.044 | 10.688 | 0.960 | 1.00 | 16.65 | O |
| ATOM | 830 | N | LEU | A | 123 | 38.262 | 11.702 | 2.989 | 1.00 | 17.86 | N |
| ATOM | 831 | CA | LEU | A | 123 | 37.478 | 10.730 | 3.713 | 1.00 | 19.18 | C |
| ATOM | 832 | CB | LEU | A | 123 | 38.198 | 10.358 | 5.021 | 1.00 | 19.08 | C |
| ATOM | 833 | CG | LEU | A | 123 | 39.560 | 9.631 | 4.902 | 1.00 | 21.69 | C |
| ATOM | 834 | CD1 | LEU | A | 123 | 39.987 | 9.066 | 6.225 | 1.00 | 24.39 | C |
| ATOM | 835 | CD2 | LEU | A | 123 | 39.492 | 8.499 | 3.907 | 1.00 | 21.24 | C |
| ATOM | 836 | C | LEU | A | 123 | 36.129 | 11.372 | 4.021 | 1.00 | 19.59 | C |
| ATOM | 837 | O | LEU | A | 123 | 36.005 | 12.615 | 3.941 | 1.00 | 18.89 | O |
| ATOM | 838 | N | PRO | A | 124 | 35.128 | 10.544 | 4.338 | 1.00 | 19.44 | N |
| ATOM | 839 | CA | PRO | A | 124 | 33.792 | 11.056 | 4.703 | 1.00 | 20.01 | C |
| ATOM | 840 | CB | PRO | A | 124 | 32.860 | 9.819 | 4.520 | 1.00 | 20.37 | C |
| ATOM | 841 | CG | PRO | A | 124 | 33.778 | 8.592 | 4.509 | 1.00 | 19.57 | C |
| ATOM | 842 | CD | PRO | A | 124 | 35.188 | 9.063 | 4.337 | 1.00 | 19.19 | C |
| ATOM | 843 | C | PRO | A | 124 | 33.730 | 11.570 | 6.150 | 1.00 | 20.18 | C |
| ATOM | 844 | O | PRO | A | 124 | 34.695 | 11.440 | 6.906 | 1.00 | 19.83 | O |
| ATOM | 845 | N | ALA | A | 125 | 32.593 | 12.156 | 6.519 | 1.00 | 22.19 | N |
| ATOM | 846 | CA | ALA | A | 125 | 32.315 | 12.546 | 7.890 | 1.00 | 22.85 | C |
| ATOM | 847 | CB | ALA | A | 125 | 31.074 | 13.459 | 7.940 | 1.00 | 23.90 | C |
| ATOM | 848 | C | ALA | A | 125 | 32.065 | 11.297 | 8.710 | 1.00 | 23.63 | C |
| ATOM | 849 | O | ALA | A | 125 | 31.568 | 10.292 | 8.198 | 1.00 | 23.68 | O |
| ATOM | 850 | N | GLN | A | 126 | 32.409 | 11.377 | 9.991 | 1.00 | 24.40 | N |
| ATOM | 851 | CA | GLN | A | 126 | 32.123 | 10.332 | 10.938 | 1.00 | 24.61 | C |
| ATOM | 852 | CB | GLN | A | 126 | 32.510 | 10.778 | 12.360 | 1.00 | 24.88 | C |
| ATOM | 853 | CG | GLN | A | 126 | 32.054 | 9.822 | 13.448 | 1.00 | 24.56 | C |
| ATOM | 854 | CD | GLN | A | 126 | 32.777 | 8.472 | 13.392 | 1.00 | 25.34 | C |
| ATOM | 855 | OE1 | GLN | A | 126 | 33.999 | 8.421 | 13.163 | 1.00 | 27.00 | O |
| ATOM | 856 | NE2 | GLN | A | 126 | 32.047 | 7.395 | 13.657 | 1.00 | 21.53 | N |
| ATOM | 857 | C | GLN | A | 126 | 30.644 | 9.976 | 10.881 | 1.00 | 24.60 | C |
| ATOM | 858 | O | GLN | A | 126 | 29.790 | 10.857 | 10.841 | 1.00 | 23.74 | O |
| ATOM | 859 | N | GLY | A | 127 | 30.381 | 8.670 | 10.819 | 1.00 | 25.19 | N |
| ATOM | 860 | CA | GLY | A | 127 | 29.053 | 8.100 | 10.850 | 1.00 | 25.64 | C |
| ATOM | 861 | C | GLY | A | 127 | 28.241 | 8.145 | 9.575 | 1.00 | 26.12 | C |
| ATOM | 862 | O | GLY | A | 127 | 27.139 | 7.617 | 9.564 | 1.00 | 26.52 | O |
| ATOM | 863 | N | ARG | A | 128 | 28.743 | 8.765 | 8.511 | 1.00 | 26.86 | N |
| ATOM | 864 | CA | ARG | A | 128 | 27.934 | 8.879 | 7.273 | 1.00 | 27.83 | C |
| ATOM | 865 | CB | ARG | A | 128 | 28.566 | 9.772 | 6.181 | 1.00 | 27.88 | C |
| ATOM | 866 | CG | ARG | A | 128 | 27.682 | 9.803 | 4.849 | 1.00 | 32.58 | C |
| ATOM | 867 | CD | ARG | A | 128 | 27.881 | 10.996 | 3.883 | 1.00 | 40.55 | C |
| ATOM | 868 | NE | ARG | A | 128 | 27.626 | 10.656 | 2.450 | 1.00 | 42.27 | N |
| ATOM | 869 | CZ | ARG | A | 128 | 28.530 | 10.763 | 1.464 | 1.00 | 39.91 | C |
| ATOM | 870 | NH1 | ARG | A | 128 | 29.744 | 11.196 | 1.741 | 1.00 | 38.53 | N |
| ATOM | 871 | NH2 | ARG | A | 128 | 28.235 | 10.403 | 0.217 | 1.00 | 38.54 | N |
| ATOM | 872 | C | ARG | A | 128 | 27.646 | 7.510 | 6.679 | 1.00 | 27.18 | C |
| ATOM | 873 | O | ARG | A | 128 | 28.552 | 6.802 | 6.286 | 1.00 | 27.06 | O |
| ATOM | 874 | N | ARG | A | 129 | 26.385 | 7.140 | 6.618 | 1.00 | 26.64 | N |
| ATOM | 875 | CA | ARG | A | 129 | 26.016 | 5.933 | 5.906 | 1.00 | 27.60 | C |
| ATOM | 876 | CB | ARG | A | 129 | 24.845 | 5.252 | 6.592 | 1.00 | 28.65 | C |
| ATOM | 877 | CG | ARG | A | 129 | 25.144 | 4.760 | 7.995 | 1.00 | 31.05 | C |
| ATOM | 878 | CD | ARG | A | 129 | 24.186 | 3.673 | 8.468 | 1.00 | 36.01 | C |
| ATOM | 879 | NE | ARG | A | 129 | 24.048 | 2.671 | 7.423 | 1.00 | 39.51 | N |
| ATOM | 880 | CZ | ARG | A | 129 | 24.705 | 1.509 | 7.404 | 1.00 | 43.53 | C |
| ATOM | 881 | NH1 | ARG | A | 129 | 25.529 | 1.196 | 8.418 | 1.00 | 41.48 | N |
| ATOM | 882 | NH2 | ARG | A | 129 | 24.523 | 0.655 | 6.380 | 1.00 | 41.90 | N |
| ATOM | 883 | C | ARG | A | 129 | 25.699 | 6.215 | 4.423 | 1.00 | 26.58 | C |
| ATOM | 884 | O | ARG | A | 129 | 25.256 | 7.306 | 4.061 | 1.00 | 26.94 | O |
| ATOM | 885 | N | LEU | A | 130 | 25.921 | 5.215 | 3.588 | 1.00 | 24.36 | N |
| ATOM | 886 | CA | LEU | A | 130 | 25.593 | 5.283 | 2.170 | 1.00 | 22.64 | C |
| ATOM | 887 | CB | LEU | A | 130 | 26.698 | 4.609 | 1.363 | 1.00 | 22.46 | C |
| ATOM | 888 | CG | LEU | A | 130 | 28.046 | 5.332 | 1.374 | 1.00 | 19.32 | C |
| ATOM | 889 | CD1 | LEU | A | 130 | 28.980 | 4.523 | 0.507 | 1.00 | 19.95 | C |
| ATOM | 890 | CD2 | LEU | A | 130 | 27.840 | 6.719 | 0.813 | 1.00 | 18.04 | C |
| ATOM | 891 | C | LEU | A | 130 | 24.287 | 4.567 | 1.892 | 1.00 | 22.63 | C |
| ATOM | 892 | O | LEU | A | 130 | 24.085 | 3.456 | 2.380 | 1.00 | 20.30 | O |
| ATOM | 893 | N | GLY | A | 131 | 23.408 | 5.214 | 1.107 | 1.00 | 22.30 | N |
| ATOM | 894 | CA | GLY | A | 131 | 22.111 | 4.628 | 0.801 | 1.00 | 22.79 | C |
| ATOM | 895 | C | GLY | A | 131 | 22.215 | 3.537 | −0.251 | 1.00 | 22.87 | C |
| ATOM | 896 | O | GLY | A | 131 | 23.127 | 3.542 | −1.067 | 1.00 | 22.14 | O |
| ATOM | 897 | N | ASN | A | 132 | 21.304 | 2.579 | −0.196 | 1.00 | 22.99 | N |
| ATOM | 898 | CA | ASN | A | 132 | 21.110 | 1.631 | −1.287 | 1.00 | 23.51 | C |
| ATOM | 899 | CB | ASN | A | 132 | 19.767 | 0.952 | −1.069 | 1.00 | 24.16 | C |
| ATOM | 900 | CG | ASN | A | 132 | 19.678 | −0.340 | −1.755 | 1.00 | 27.74 | C |
| ATOM | 901 | OD1 | ASN | A | 132 | 20.350 | −1.304 | −1.365 | 1.00 | 30.71 | O |
| ATOM | 902 | ND2 | ASN | A | 132 | 18.854 | −0.399 | −2.814 | 1.00 | 31.30 | N |
| ATOM | 903 | C | ASN | A | 132 | 21.098 | 2.329 | −2.656 | 1.00 | 22.26 | C |
| ATOM | 904 | O | ASN | A | 132 | 20.363 | 3.290 | −2.852 | 1.00 | 23.27 | O |
| ATOM | 905 | N | GLY | A | 133 | 21.900 | 1.865 | −3.606 | 1.00 | 20.97 | N |
| ATOM | 906 | CA | GLY | A | 133 | 21.939 | 2.508 | −4.914 | 1.00 | 18.28 | C |
| ATOM | 907 | C | GLY | A | 133 | 23.036 | 3.537 | −5.151 | 1.00 | 17.85 | C |
| ATOM | 908 | O | GLY | A | 133 | 23.209 | 3.986 | −6.264 | 1.00 | 16.85 | O |
| ATOM | 909 | N | VAL | A | 134 | 23.796 | 3.898 | −4.123 | 1.00 | 16.98 | N |
| ATOM | 910 | CA | VAL | A | 134 | 24.923 | 4.813 | −4.323 | 1.00 | 17.17 | C |
| ATOM | 911 | CB | VAL | A | 134 | 25.606 | 5.206 | −2.985 | 1.00 | 17.52 | C |
| ATOM | 912 | CG1 | VAL | A | 134 | 27.014 | 5.737 | −3.215 | 1.00 | 16.92 | C |
| ATOM | 913 | CG2 | VAL | A | 134 | 24.747 | 6.259 | −2.253 | 1.00 | 17.21 | C |
| ATOM | 914 | C | VAL | A | 134 | 25.919 | 4.160 | −5.285 | 1.00 | 17.74 | C |
| ATOM | 915 | O | VAL | A | 134 | 26.135 | 2.929 | −5.238 | 1.00 | 16.57 | O |
| ATOM | 916 | N | GLN | A | 135 | 26.459 | 4.968 | −6.190 | 1.00 | 17.24 | N |
| ATOM | 917 | CA | GLN | A | 135 | 27.341 | 4.461 | −7.243 | 1.00 | 18.62 | C |
| ATOM | 918 | CB | GLN | A | 135 | 27.120 | 5.195 | −8.571 | 1.00 | 18.52 | C |
| ATOM | 919 | CG | GLN | A | 135 | 25.773 | 5.041 | −9.201 | 1.00 | 24.15 | C |
| ATOM | 920 | CD | GLN | A | 135 | 25.437 | 3.613 | −9.391 | 1.00 | 30.83 | C |
| ATOM | 921 | OE1 | GLN | A | 135 | 26.033 | 2.938 | −10.240 | 1.00 | 33.13 | O |
| ATOM | 922 | NE2 | GLN | A | 135 | 24.506 | 3.115 | −8.579 | 1.00 | 32.07 | N |
| ATOM | 923 | C | GLN | A | 135 | 28.772 | 4.696 | −6.801 | 1.00 | 16.65 | C |
| ATOM | 924 | O | GLN | A | 135 | 29.146 | 5.825 | −6.432 | 1.00 | 15.72 | O |
| ATOM | 925 | N | CYS | A | 136 | 29.572 | 3.637 | −6.845 | 1.00 | 16.31 | N |
| ATOM | 926 | CA | CYS | A | 136 | 30.977 | 3.753 | −6.473 | 1.00 | 15.03 | C |
| ATOM | 927 | CB | CYS | A | 136 | 31.223 | 3.078 | −5.132 | 1.00 | 15.13 | C |
| ATOM | 928 | SG | CYS | A | 136 | 30.101 | 3.507 | −3.797 | 1.00 | 15.57 | S |
| ATOM | 929 | C | CYS | A | 136 | 31.903 | 3.108 | −7.473 | 1.00 | 15.10 | C |
| ATOM | 930 | O | CYS | A | 136 | 31.473 | 2.434 | −8.396 | 1.00 | 14.12 | O |
| ATOM | 931 | N | LEU | A | 137 | 33.198 | 3.336 | −7.258 | 1.00 | 13.88 | N |
| ATOM | 932 | CA | LEU | A | 137 | 34.247 | 2.680 | −7.991 | 1.00 | 13.98 | C |
| ATOM | 933 | CB | LEU | A | 137 | 35.128 | 3.730 | −8.617 | 1.00 | 14.51 | C |
| ATOM | 934 | CG | LEU | A | 137 | 35.745 | 3.544 | −9.991 | 1.00 | 18.13 | C |
| ATOM | 935 | CD1 | LEU | A | 137 | 34.752 | 3.025 | −11.084 | 1.00 | 14.73 | C |
| ATOM | 936 | CD2 | LEU | A | 137 | 36.283 | 4.940 | −10.337 | 1.00 | 20.13 | C |
| ATOM | 937 | C | LEU | A | 137 | 35.077 | 1.876 | −6.994 | 1.00 | 12.54 | C |
| ATOM | 938 | O | LEU | A | 137 | 35.605 | 2.433 | −6.011 | 1.00 | 12.23 | O |
| ATOM | 939 | N | ALA | A | 138 | 35.169 | 0.584 | −7.249 | 1.00 | 11.40 | N |
| ATOM | 940 | CA | ALA | A | 138 | 36.174 | −0.292 | −6.616 | 1.00 | 11.08 | C |
| ATOM | 941 | CB | ALA | A | 138 | 35.592 | −1.689 | −6.429 | 1.00 | 9.92 | C |
| ATOM | 942 | C | ALA | A | 138 | 37.424 | −0.339 | −7.502 | 1.00 | 10.67 | C |
| ATOM | 943 | O | ALA | A | 138 | 37.350 | −0.042 | −8.707 | 1.00 | 11.95 | O |
| ATOM | 944 | N | MET | A | 139 | 38.578 | −0.683 | −6.928 | 1.00 | 10.38 | N |
| ATOM | 945 | CA | MET | A | 139 | 39.805 | −0.721 | −7.692 | 1.00 | 10.28 | C |
| ATOM | 946 | CB | MET | A | 139 | 40.406 | 0.709 | −7.883 | 1.00 | 10.19 | C |
| ATOM | 947 | CG | MET | A | 139 | 40.675 | 1.431 | −6.513 | 1.00 | 8.95 | C |
| ATOM | 948 | SD | MET | A | 139 | 41.240 | 3.140 | −6.780 | 1.00 | 13.25 | S |
| ATOM | 949 | CE | MET | A | 139 | 39.641 | 3.827 | −7.294 | 1.00 | 9.10 | C |
| ATOM | 950 | C | MET | A | 139 | 40.827 | −1.610 | −7.023 | 1.00 | 11.26 | C |
| ATOM | 951 | O | MET | A | 139 | 40.715 | −1.931 | −5.837 | 1.00 | 12.11 | O |
| ATOM | 952 | N | GLY | A | 140 | 41.843 | −2.001 | −7.781 | 1.00 | 11.51 | N |
| ATOM | 953 | CA | GLY | A | 140 | 42.906 | −2.769 | −7.191 | 1.00 | 11.72 | C |
| ATOM | 954 | C | GLY | A | 140 | 43.723 | −3.502 | −8.218 | 1.00 | 11.68 | C |
| ATOM | 955 | O | GLY | A | 140 | 43.407 | −3.501 | −9.440 | 1.00 | 10.56 | O |
| ATOM | 956 | N | TRP | A | 141 | 44.803 | −4.091 | −7.714 | 1.00 | 11.34 | N |
| ATOM | 957 | CA | TRP | A | 141 | 45.693 | −4.927 | −8.542 | 1.00 | 13.33 | C |
| ATOM | 958 | CB | TRP | A | 141 | 47.157 | −4.605 | −8.278 | 1.00 | 13.41 | C |
| ATOM | 959 | CG | TRP | A | 141 | 47.651 | −3.316 | −8.935 | 1.00 | 15.26 | C |
| ATOM | 960 | CD1 | TRP | A | 141 | 48.005 | −3.141 | −10.241 | 1.00 | 13.90 | C |
| ATOM | 961 | NE1 | TRP | A | 141 | 48.466 | −1.858 | −10.443 | 1.00 | 16.96 | N |
| ATOM | 962 | CE2 | TRP | A | 141 | 48.392 | −1.164 | −9.262 | 1.00 | 15.49 | C |
| ATOM | 963 | CD2 | TRP | A | 141 | 47.875 | −2.062 | −8.283 | 1.00 | 15.17 | C |
| ATOM | 964 | CE3 | TRP | A | 141 | 47.684 | −1.592 | −6.963 | 1.00 | 13.85 | C |
| ATOM | 965 | CZ3 | TRP | A | 141 | 48.026 | −0.250 | −6.673 | 1.00 | 13.54 | C |
| ATOM | 966 | CH2 | TRP | A | 141 | 48.530 | 0.630 | −7.696 | 1.00 | 10.71 | C |
| ATOM | 967 | CZ2 | TRP | A | 141 | 48.705 | 0.194 | −8.979 | 1.00 | 12.78 | C |
| ATOM | 968 | C | TRP | A | 141 | 45.493 | −6.431 | −8.305 | 1.00 | 13.99 | C |
| ATOM | 969 | O | TRP | A | 141 | 46.376 | −7.213 | −8.666 | 1.00 | 15.18 | O |
| ATOM | 970 | N | GLY | A | 142 | 44.362 | −6.807 | −7.691 | 1.00 | 14.03 | N |
| ATOM | 971 | CA | GLY | A | 142 | 44.010 | −8.201 | −7.426 | 1.00 | 14.50 | C |
| ATOM | 972 | C | GLY | A | 142 | 43.745 | −9.096 | −8.629 | 1.00 | 15.22 | C |
| ATOM | 973 | O | GLY | A | 142 | 43.906 | −8.708 | −9.790 | 1.00 | 13.57 | O |
| ATOM | 974 | N | LEU | A | 143 | 43.356 | −10.336 | −8.339 | 1.00 | 15.72 | N |
| ATOM | 975 | CA | LEU | A | 143 | 43.150 | −11.338 | −9.390 | 1.00 | 15.91 | C |
| ATOM | 976 | CB | LEU | A | 143 | 42.759 | −12.708 | −8.797 | 1.00 | 15.67 | C |
| ATOM | 977 | CG | LEU | A | 143 | 43.752 | −13.328 | −7.800 | 1.00 | 17.45 | C |
| ATOM | 978 | CD1 | LEU | A | 143 | 43.240 | −14.717 | −7.297 | 1.00 | 17.95 | C |
| ATOM | 979 | CD2 | LEU | A | 143 | 45.152 | −13.420 | −8.391 | 1.00 | 13.17 | C |
| ATOM | 980 | C | LEU | A | 143 | 42.095 | −10.880 | −10.392 | 1.00 | 15.85 | C |
| ATOM | 981 | O | LEU | A | 143 | 41.079 | −10.273 | −10.019 | 1.00 | 14.89 | O |
| ATOM | 982 | N | LEU | A | 144 | 42.365 | −11.173 | −11.653 | 1.00 | 16.44 | N |
| ATOM | 983 | CA | LEU | A | 144 | 41.472 | −10.805 | −12.738 | 1.00 | 18.55 | C |
| ATOM | 984 | CB | LEU | A | 144 | 42.298 | −10.523 | −13.991 | 1.00 | 17.78 | C |
| ATOM | 985 | CG | LEU | A | 144 | 43.334 | −9.407 | −13.894 | 1.00 | 17.39 | C |
| ATOM | 986 | CD1 | LEU | A | 144 | 44.436 | −9.547 | −14.971 | 1.00 | 23.80 | C |
| ATOM | 987 | CD2 | LEU | A | 144 | 42.647 | −8.028 | −14.018 | 1.00 | 17.25 | C |
| ATOM | 988 | C | LEU | A | 144 | 40.386 | −11.857 | −13.007 | 1.00 | 20.57 | C |
| ATOM | 989 | O | LEU | A | 144 | 39.423 | −11.585 | −13.723 | 1.00 | 20.21 | O |
| ATOM | 990 | N | GLY | A | 145 | 40.522 | −13.032 | −12.393 | 1.00 | 22.65 | N |
| ATOM | 991 | CA | GLY | A | 145 | 39.826 | −14.259 | −12.800 | 1.00 | 27.83 | C |
| ATOM | 992 | C | GLY | A | 145 | 40.562 | −15.424 | −12.113 | 1.00 | 30.70 | C |
| ATOM | 993 | O | GLY | A | 145 | 41.736 | −15.254 | −11.707 | 1.00 | 30.68 | O |
| ATOM | 994 | N | ARG | A | 147 | 40.021 | −16.646 | −12.090 | 1.00 | 33.62 | N |
| ATOM | 995 | CA | ARG | A | 147 | 39.704 | −17.472 | −13.253 | 1.00 | 35.91 | C |
| ATOM | 996 | CB | ARG | A | 147 | 38.955 | −16.726 | −14.358 | 1.00 | 37.41 | C |
| ATOM | 997 | CG | ARG | A | 147 | 38.367 | −17.656 | −15.420 | 1.00 | 41.61 | C |
| ATOM | 998 | CD | ARG | A | 147 | 36.933 | −18.129 | −15.122 | 1.00 | 47.81 | C |
| ATOM | 999 | NE | ARG | A | 147 | 35.951 | −17.216 | −15.712 | 1.00 | 52.65 | N |
| ATOM | 1000 | CZ | ARG | A | 147 | 34.640 | −17.310 | −15.544 | 1.00 | 55.53 | C |
| ATOM | 1001 | NH1 | ARG | A | 147 | 34.139 | −18.289 | −14.799 | 1.00 | 57.09 | N |
| ATOM | 1002 | NH2 | ARG | A | 147 | 33.826 | −16.428 | −16.121 | 1.00 | 55.95 | N |
| ATOM | 1003 | C | ARG | A | 147 | 41.100 | −17.901 | −13.727 | 1.00 | 35.65 | C |
| ATOM | 1004 | O | ARG | A | 147 | 41.369 | −17.906 | −14.920 | 1.00 | 36.14 | O |
| ATOM | 1005 | N | ASN | A | 148 | 41.978 | −18.230 | −12.763 | 1.00 | 35.21 | N |
| ATOM | 1006 | CA | ASN | A | 148 | 43.434 | −18.385 | −12.982 | 1.00 | 35.59 | C |
| ATOM | 1007 | CB | ASN | A | 148 | 43.829 | −19.829 | −13.368 | 1.00 | 35.49 | C |
| ATOM | 1008 | CG | ASN | A | 148 | 43.718 | −20.824 | −12.217 | 1.00 | 38.01 | C |
| ATOM | 1009 | OD1 | ASN | A | 148 | 43.589 | −20.477 | −11.046 | 1.00 | 38.33 | O |
| ATOM | 1010 | ND2 | ASN | A | 148 | 43.758 | −22.099 | −12.572 | 1.00 | 43.98 | N |
| ATOM | 1011 | C | ASN | A | 148 | 44.093 | −17.393 | −13.988 | 1.00 | 34.84 | C |
| ATOM | 1012 | O | ASN | A | 148 | 45.003 | −17.782 | −14.703 | 1.00 | 34.22 | O |
| ATOM | 1013 | N | ARG | A | 149 | 43.646 | −16.130 | −14.016 | 1.00 | 34.12 | N |
| ATOM | 1014 | CA | ARG | A | 149 | 44.273 | −15.095 | −14.849 | 1.00 | 33.47 | C |
| ATOM | 1015 | CB | ARG | A | 149 | 43.233 | −14.075 | −15.347 | 1.00 | 34.35 | C |
| ATOM | 1016 | CG | ARG | A | 149 | 42.824 | −14.206 | −16.840 | 1.00 | 37.62 | C |
| ATOM | 1017 | CD | ARG | A | 149 | 41.806 | −13.124 | −17.354 | 1.00 | 41.76 | C |
| ATOM | 1018 | NE | ARG | A | 149 | 40.430 | −13.447 | −16.940 | 1.00 | 45.31 | N |
| ATOM | 1019 | CZ | ARG | A | 149 | 39.593 | −14.263 | −17.604 | 1.00 | 48.84 | C |
| ATOM | 1020 | NH1 | ARG | A | 149 | 39.957 | −14.842 | −18.751 | 1.00 | 48.76 | N |
| ATOM | 1021 | NH2 | ARG | A | 149 | 38.374 | −14.505 | −17.118 | 1.00 | 50.93 | N |
| ATOM | 1022 | C | ARG | A | 149 | 45.409 | −14.390 | −14.090 | 1.00 | 31.75 | C |
| ATOM | 1023 | O | ARG | A | 149 | 46.178 | −13.632 | −14.662 | 1.00 | 31.56 | O |
| ATOM | 1024 | N | GLY | A | 150 | 45.515 | −14.656 | −12.797 | 1.00 | 30.39 | N |
| ATOM | 1025 | CA | GLY | A | 150 | 46.565 | −14.082 | −11.976 | 1.00 | 28.27 | C |
| ATOM | 1026 | C | GLY | A | 150 | 46.300 | −12.625 | −11.622 | 1.00 | 27.37 | C |
| ATOM | 1027 | O | GLY | A | 150 | 45.236 | −12.059 | −11.897 | 1.00 | 26.35 | O |
| ATOM | 1028 | N | ILE | A | 151 | 47.292 | −12.032 | −10.982 | 1.00 | 25.45 | N |
| ATOM | 1029 | CA | ILE | A | 151 | 47.225 | −10.677 | −10.491 | 1.00 | 24.16 | C |
| ATOM | 1030 | CB | ILE | A | 151 | 48.350 | −10.590 | −9.427 | 1.00 | 25.14 | C |
| ATOM | 1031 | CG1 | ILE | A | 151 | 47.904 | −9.805 | −8.219 | 1.00 | 26.61 | C |
| ATOM | 1032 | CD1 | ILE | A | 151 | 47.218 | −10.687 | −7.219 | 1.00 | 30.13 | C |
| ATOM | 1033 | CG2 | ILE | A | 151 | 49.727 | −10.222 | −10.016 | 1.00 | 26.23 | C |
| ATOM | 1034 | C | ILE | A | 151 | 47.300 | −9.637 | −11.676 | 1.00 | 23.02 | C |
| ATOM | 1035 | O | ILE | A | 151 | 47.852 | −9.927 | −12.737 | 1.00 | 21.49 | O |
| ATOM | 1036 | N | ALA | A | 152 | 46.711 | −8.456 | −11.521 | 1.00 | 20.91 | N |
| ATOM | 1037 | CA | ALA | A | 152 | 46.751 | −7.502 | −12.629 | 1.00 | 20.14 | C |
| ATOM | 1038 | CB | ALA | A | 152 | 45.642 | −6.463 | −12.503 | 1.00 | 19.41 | C |
| ATOM | 1039 | C | ALA | A | 152 | 48.118 | −6.823 | −12.651 | 1.00 | 19.84 | C |
| ATOM | 1040 | O | ALA | A | 152 | 48.723 | −6.581 | −11.600 | 1.00 | 20.24 | O |
| ATOM | 1041 | N | SER | A | 153 | 48.611 | −6.524 | −13.840 | 1.00 | 19.65 | N |
| ATOM | 1042 | CA | SER | A | 153 | 49.774 | −5.654 | −13.946 | 1.00 | 19.60 | C |
| ATOM | 1043 | CB | SER | A | 153 | 50.475 | −5.855 | −15.286 | 1.00 | 20.67 | C |
| ATOM | 1044 | OG | SER | A | 153 | 51.051 | −7.154 | −15.265 | 1.00 | 23.47 | O |
| ATOM | 1045 | C | SER | A | 153 | 49.307 | −4.225 | −13.799 | 1.00 | 18.75 | C |
| ATOM | 1046 | O | SER | A | 153 | 49.811 | −3.484 | −12.960 | 1.00 | 18.45 | O |
| ATOM | 1047 | N | VAL | A | 154 | 48.304 | −3.842 | −14.591 | 1.00 | 17.71 | N |
| ATOM | 1048 | CA | VAL | A | 154 | 47.868 | −2.477 | −14.484 | 1.00 | 16.95 | C |
| ATOM | 1049 | CB | VAL | A | 154 | 47.775 | −1.718 | −15.843 | 1.00 | 18.46 | C |
| ATOM | 1050 | CG1 | VAL | A | 154 | 48.504 | −2.447 | −16.988 | 1.00 | 19.00 | C |
| ATOM | 1051 | CG2 | VAL | A | 154 | 46.404 | −1.311 | −16.176 | 1.00 | 19.72 | C |
| ATOM | 1052 | C | VAL | A | 154 | 46.640 | −2.323 | −13.588 | 1.00 | 14.59 | C |
| ATOM | 1053 | O | VAL | A | 154 | 45.721 | −3.177 | −13.607 | 1.00 | 12.70 | O |
| ATOM | 1054 | N | LEU | A | 155 | 46.667 | −1.253 | −12.782 | 1.00 | 12.29 | N |
| ATOM | 1055 | CA | LEU | A | 155 | 45.580 | −0.977 | −11.874 | 1.00 | 11.36 | C |
| ATOM | 1056 | CB | LEU | A | 155 | 45.762 | 0.417 | −11.247 | 1.00 | 12.24 | C |
| ATOM | 1057 | CG | LEU | A | 155 | 44.694 | 0.827 | −10.215 | 1.00 | 11.82 | C |
| ATOM | 1058 | CD1 | LEU | A | 155 | 44.728 | −0.087 | −8.933 | 1.00 | 9.81 | C |
| ATOM | 1059 | CD2 | LEU | A | 155 | 44.881 | 2.311 | −9.867 | 1.00 | 10.76 | C |
| ATOM | 1060 | C | LEU | A | 155 | 44.221 | −1.105 | −12.578 | 1.00 | 11.45 | C |
| ATOM | 1061 | O | LEU | A | 155 | 43.997 | −0.509 | −13.662 | 1.00 | 10.98 | O |
| ATOM | 1062 | N | GLN | A | 156 | 43.317 | −1.882 | −11.975 | 1.00 | 10.79 | N |
| ATOM | 1063 | CA | GLN | A | 156 | 41.947 | −2.025 | −12.487 | 1.00 | 11.83 | C |
| ATOM | 1064 | CB | GLN | A | 156 | 41.551 | −3.482 | −12.446 | 1.00 | 11.09 | C |
| ATOM | 1065 | CG | GLN | A | 156 | 42.584 | −4.368 | −13.132 | 1.00 | 13.03 | C |
| ATOM | 1066 | CD | GLN | A | 156 | 42.488 | −4.303 | −14.673 | 1.00 | 12.91 | C |
| ATOM | 1067 | OE1 | GLN | A | 156 | 43.499 | −4.023 | −15.355 | 1.00 | 16.43 | O |
| ATOM | 1068 | NE2 | GLN | A | 156 | 41.301 | −4.567 | −15.207 | 1.00 | 9.79 | N |
| ATOM | 1069 | C | GLN | A | 156 | 40.937 | −1.226 | −11.675 | 1.00 | 12.23 | C |
| ATOM | 1070 | O | GLN | A | 156 | 41.131 | −0.992 | −10.483 | 1.00 | 11.82 | O |
| ATOM | 1071 | N | GLU | A | 157 | 39.856 | −0.807 | −12.319 | 1.00 | 12.33 | N |
| ATOM | 1072 | CA | GLU | A | 157 | 38.735 | −0.204 | −11.625 | 1.00 | 12.62 | C |
| ATOM | 1073 | CB | GLU | A | 157 | 38.659 | 1.307 | −11.880 | 1.00 | 14.19 | C |
| ATOM | 1074 | CG | GLU | A | 157 | 38.655 | 1.706 | −13.346 | 1.00 | 11.10 | C |
| ATOM | 1075 | CD | GLU | A | 157 | 38.688 | 3.212 | −13.531 | 1.00 | 15.45 | C |
| ATOM | 1076 | OE1 | GLU | A | 157 | 39.673 | 3.832 | −13.103 | 1.00 | 15.11 | O |
| ATOM | 1077 | OE2 | GLU | A | 157 | 37.735 | 3.771 | −14.122 | 1.00 | 17.14 | O |
| ATOM | 1078 | C | GLU | A | 157 | 37.440 | −0.844 | −12.091 | 1.00 | 13.15 | C |
| ATOM | 1079 | O | GLU | A | 157 | 37.408 | −1.494 | −13.115 | 1.00 | 12.57 | O |
| ATOM | 1080 | N | LEU | A | 158 | 36.362 | −0.603 | −11.346 | 1.00 | 13.22 | N |
| ATOM | 1081 | CA | LEU | A | 158 | 35.127 | −1.299 | −11.565 | 1.00 | 13.18 | C |
| ATOM | 1082 | CB | LEU | A | 158 | 35.158 | −2.702 | −10.894 | 1.00 | 12.31 | C |
| ATOM | 1083 | CG | LEU | A | 158 | 33.853 | −3.493 | −10.926 | 1.00 | 12.36 | C |
| ATOM | 1084 | CD1 | LEU | A | 158 | 33.436 | −3.897 | −12.372 | 1.00 | 11.06 | C |
| ATOM | 1085 | CD2 | LEU | A | 158 | 33.992 | −4.708 | −10.084 | 1.00 | 9.88 | C |
| ATOM | 1086 | C | LEU | A | 158 | 33.987 | −0.472 | −10.991 | 1.00 | 12.95 | C |
| ATOM | 1087 | O | LEU | A | 158 | 34.013 | −0.096 | −9.806 | 1.00 | 12.66 | O |
| ATOM | 1088 | N | ASN | A | 159 | 32.979 | −0.210 | −11.827 | 1.00 | 12.52 | N |
| ATOM | 1089 | CA | ASN | A | 159 | 31.768 | 0.437 | −11.343 | 1.00 | 13.26 | C |
| ATOM | 1090 | CB | ASN | A | 159 | 30.895 | 0.921 | −12.488 | 1.00 | 13.80 | C |
| ATOM | 1091 | CG | ASN | A | 159 | 31.410 | 2.177 | −13.145 | 1.00 | 14.92 | C |
| ATOM | 1092 | OD1 | ASN | A | 159 | 32.483 | 2.202 | −13.765 | 1.00 | 15.93 | O |
| ATOM | 1093 | ND2 | ASN | A | 159 | 30.606 | 3.228 | −13.031 | 1.00 | 17.32 | N |
| ATOM | 1094 | C | ASN | A | 159 | 30.978 | −0.555 | −10.534 | 1.00 | 13.45 | C |
| ATOM | 1095 | O | ASN | A | 159 | 30.688 | −1.666 | −10.976 | 1.00 | 13.71 | O |
| ATOM | 1096 | N | VAL | A | 160 | 30.548 | −0.131 | −9.366 | 1.00 | 13.42 | N |
| ATOM | 1097 | CA | VAL | A | 160 | 29.866 | −1.019 | −8.466 | 1.00 | 14.38 | C |
| ATOM | 1098 | CB | VAL | A | 160 | 31.050 | −1.475 | −7.553 | 1.00 | 16.29 | C |
| ATOM | 1099 | CG1 | VAL | A | 160 | 31.084 | −0.882 | −6.159 | 1.00 | 12.78 | C |
| ATOM | 1100 | CG2 | VAL | A | 160 | 31.577 | −2.867 | −7.828 | 1.00 | 13.76 | C |
| ATOM | 1101 | C | VAL | A | 160 | 28.735 | −0.230 | −7.763 | 1.00 | 14.88 | C |
| ATOM | 1102 | O | VAL | A | 160 | 28.784 | 1.002 | −7.706 | 1.00 | 15.05 | O |
| ATOM | 1103 | N | THR | A | 162 | 27.737 | −0.932 | −7.209 | 1.00 | 15.89 | N |
| ATOM | 1104 | CA | THR | A | 162 | 26.566 | −0.281 | −6.610 | 1.00 | 16.18 | C |
| ATOM | 1105 | CB | THR | A | 162 | 25.281 | −0.671 | −7.428 | 1.00 | 16.62 | C |
| ATOM | 1106 | OG1 | THR | A | 162 | 25.318 | −0.025 | −8.708 | 1.00 | 17.50 | O |
| ATOM | 1107 | CG2 | THR | A | 162 | 24.008 | −0.077 | −6.768 | 1.00 | 17.24 | C |
| ATOM | 1108 | C | THR | A | 162 | 26.381 | −0.686 | −5.152 | 1.00 | 16.01 | C |
| ATOM | 1109 | O | THR | A | 162 | 26.324 | −1.884 | −4.835 | 1.00 | 15.26 | O |
| ATOM | 1110 | N | VAL | A | 163 | 26.238 | 0.290 | −4.265 | 1.00 | 15.52 | N |
| ATOM | 1111 | CA | VAL | A | 163 | 26.007 | −0.030 | −2.852 | 1.00 | 16.03 | C |
| ATOM | 1112 | CB | VAL | A | 163 | 26.023 | 1.227 | −1.937 | 1.00 | 16.61 | C |
| ATOM | 1113 | CG1 | VAL | A | 1G3 | 25.654 | 0.859 | −0.476 | 1.00 | 16.32 | C |
| ATOM | 1114 | CG2 | VAL | A | 163 | 27.391 | 1.954 | −1.979 | 1.00 | 14.75 | C |
| ATOM | 1115 | C | VAL | A | 163 | 24.689 | −0.762 | −2.674 | 1.00 | 17.29 | C |
| ATOM | 1116 | O | VAL | A | 163 | 23.639 | −0.318 | −3.187 | 1.00 | 17.52 | O |
| ATOM | 1117 | N | VAL | A | 164 | 24.747 | −1.893 | −1.981 | 1.00 | 17.30 | N |
| ATOM | 1118 | CA | VAL | A | 164 | 23.535 | −2.590 | −1.573 | 1.00 | 19.31 | C |
| ATOM | 1119 | CB | VAL | A | 164 | 23.363 | −3.991 | −2.267 | 1.00 | 19.37 | C |
| ATOM | 1120 | CG1 | VAL | A | 164 | 23.083 | −3.809 | −3.730 | 1.00 | 18.80 | C |
| ATOM | 1121 | CG2 | VAL | A | 164 | 24.614 | −4.872 | −2.034 | 1.00 | 17.88 | C |
| ATOM | 1122 | C | VAL | A | 164 | 23.504 | −2.766 | −0.061 | 1.00 | 19.91 | C |
| ATOM | 1123 | O | VAL | A | 164 | 24.545 | −2.944 | 0.590 | 1.00 | 20.22 | O |
| ATOM | 1124 | N | THR | A | 165 | 22.303 | −2.714 | 0.507 | 1.00 | 21.15 | N |
| ATOM | 1125 | CA | THR | A | 165 | 22.152 | −3.077 | 1.933 | 1.00 | 22.49 | C |
| ATOM | 1126 | CB | THR | A | 165 | 21.266 | −2.067 | 2.692 | 1.00 | 22.49 | C |
| ATOM | 1127 | OG1 | THR | A | 165 | 19.998 | −1.968 | 2.014 | 1.00 | 22.21 | O |
| ATOM | 1128 | CG2 | THR | A | 165 | 21.882 | −0.651 | 2.608 | 1.00 | 21.24 | C |
| ATOM | 1129 | C | THR | A | 165 | 21.634 | −4.501 | 2.139 | 1.00 | 23.27 | C |
| ATOM | 1130 | O | THR | A | 165 | 21.881 | −5.092 | 3.182 | 1.00 | 24.34 | O |
| ATOM | 1131 | N | SER | A | 166 | 20.961 | −5.063 | 1.146 | 1.00 | 24.54 | N |
| ATOM | 1132 | CA | SER | A | 166 | 20.535 | −6.480 | 1.182 | 1.00 | 26.11 | C |
| ATOM | 1133 | CB | SER | A | 166 | 19.574 | −6.773 | 0.036 | 1.00 | 25.78 | C |
| ATOM | 1134 | OG | SER | A | 166 | 18.693 | −7.835 | 0.382 | 1.00 | 26.25 | O |
| ATOM | 1135 | C | SER | A | 166 | 21.707 | −7.474 | 1.133 | 1.00 | 27.08 | C |
| ATOM | 1136 | O | SER | A | 166 | 22.655 | −7.287 | 0.364 | 1.00 | 28.14 | O |
| ATOM | 1137 | N | LEU | A | 167 | 21.646 | −8.512 | 1.959 | 1.00 | 27.52 | N |
| ATOM | 1138 | CA | LEU | A | 167 | 22.697 | −9.513 | 2.041 | 1.00 | 28.57 | C |
| ATOM | 1139 | CB | LEU | A | 167 | 22.873 | −10.204 | 0.666 | 1.00 | 28.76 | C |
| ATOM | 1140 | CG | LEU | A | 167 | 22.758 | −11.724 | 0.416 | 1.00 | 30.51 | C |
| ATOM | 1141 | CD1 | LEU | A | 167 | 21.793 | −12.479 | 1.363 | 1.00 | 30.46 | C |
| ATOM | 1142 | CD2 | LEU | A | 167 | 22.414 | −12.007 | −1.068 | 1.00 | 29.35 | C |
| ATOM | 1143 | C | LEU | A | 167 | 24.013 | −8.879 | 2.573 | 1.00 | 29.34 | C |
| ATOM | 1144 | O | LEU | A | 167 | 25.112 | −9.405 | 2.318 | 1.00 | 29.67 | O |
| ATOM | 1145 | N | CYS | A | 168 | 23.870 | −7.765 | 3.325 | 1.00 | 28.09 | N |
| ATOM | 1146 | CA | CYS | A | 168 | 24.977 | −7.042 | 3.963 | 1.00 | 27.61 | C |
| ATOM | 1147 | CB | CYS | A | 168 | 25.166 | −5.681 | 3.300 | 1.00 | 25.32 | C |
| ATOM | 1148 | SG | CYS | A | 168 | 26.753 | −4.936 | 3.689 | 1.00 | 21.80 | S |
| ATOM | 1149 | C | CYS | A | 168 | 24.709 | −6.786 | 5.439 | 1.00 | 28.77 | C |
| ATOM | 1150 | O | CYS | A | 168 | 23.583 | −6.547 | 5.815 | 1.00 | 30.89 | O |
| ATOM | 1151 | N | ARG | A | 177 | 25.740 | −6.810 | 6.274 | 1.00 | 30.50 | N |
| ATOM | 1152 | CA | ARG | A | 177 | 25.603 | −6.394 | 7.671 | 1.00 | 30.75 | C |
| ATOM | 1153 | CB | ARG | A | 177 | 26.690 | −7.027 | 8.525 | 1.00 | 31.05 | C |
| ATOM | 1154 | CG | ARG | A | 177 | 26.700 | −8.538 | 8.522 | 1.00 | 34.06 | C |
| ATOM | 1155 | CD | ARG | A | 177 | 27.940 | −9.125 | 9.136 | 1.00 | 38.44 | C |
| ATOM | 1156 | NE | ARG | A | 177 | 29.115 | −8.298 | 8.870 | 1.00 | 41.20 | N |
| ATOM | 1157 | CZ | ARG | A | 177 | 30.279 | −8.476 | 9.462 | 1.00 | 41.19 | C |
| ATOM | 1158 | NH1 | ARG | A | 177 | 30.400 | −9.447 | 10.359 | 1.00 | 42.77 | N |
| ATOM | 1159 | NH2 | ARG | A | 177 | 31.311 | −7.691 | 9.172 | 1.00 | 39.70 | N |
| ATOM | 1160 | C | ARG | A | 177 | 25.713 | −4.884 | 7.785 | 1.00 | 31.33 | C |
| ATOM | 1161 | O | ARG | A | 177 | 26.326 | −4.233 | 6.922 | 1.00 | 30.98 | O |
| ATOM | 1162 | N | ARG | A | 178 | 25.152 | −4.320 | 8.861 | 1.00 | 30.87 | N |
| ATOM | 1163 | CA | ARG | A | 178 | 25.209 | −2.871 | 9.078 | 1.00 | 30.55 | C |
| ATOM | 1164 | CB | ARG | A | 178 | 24.265 | −2.430 | 10.210 | 1.00 | 31.71 | C |
| ATOM | 1165 | CG | ARG | A | 178 | 22.760 | −2.460 | 9.855 | 1.00 | 34.95 | C |
| ATOM | 1166 | CD | ARG | A | 178 | 22.264 | −1.371 | 8.872 | 1.00 | 39.98 | C |
| ATOM | 1167 | NE | ARG | A | 178 | 22.096 | 0.002 | 9.396 | 1.00 | 41.73 | N |
| ATOM | 1168 | CZ | ARG | A | 178 | 22.026 | 0.360 | 10.670 | 1.00 | 42.27 | C |
| ATOM | 1169 | NH1 | ARG | A | 178 | 21.883 | 1.642 | 10.976 | 1.00 | 42.55 | N |
| ATOM | 1170 | NH2 | ARG | A | 178 | 22.091 | −0.550 | 11.641 | 1.00 | 45.91 | N |
| ATOM | 1171 | C | ARG | A | 178 | 26.612 | −2.447 | 9.435 | 1.00 | 28.83 | C |
| ATOM | 1172 | O | ARG | A | 178 | 26.911 | −1.261 | 9.437 | 1.00 | 29.59 | O |
| ATOM | 1173 | N | SER | A | 179 | 27.447 | −3.423 | 9.777 | 1.00 | 26.58 | N |
| ATOM | 1174 | CA | SER | A | 179 | 28.854 | −3.223 | 10.090 | 1.00 | 25.21 | C |
| ATOM | 1175 | CB | SER | A | 179 | 29.285 | −4.271 | 11.099 | 1.00 | 25.09 | C |
| ATOM | 1176 | OG | SER | A | 179 | 29.208 | −5.556 | 10.505 | 1.00 | 27.85 | O |
| ATOM | 1177 | C | SER | A | 179 | 29.782 | −3.274 | 8.851 | 1.00 | 23.61 | C |
| ATOM | 1178 | O | SER | A | 179 | 31.015 | −3.264 | 8.981 | 1.00 | 23.36 | O |
| ATOM | 1179 | N | ASN | A | 180 | 29.189 | −3.332 | 7.662 | 1.00 | 21.29 | N |
| ATOM | 1180 | CA | ASN | A | 180 | 29.952 | −3.254 | 6.422 | 1.00 | 20.11 | C |
| ATOM | 1181 | CB | ASN | A | 180 | 30.013 | −4.606 | 5.713 | 1.00 | 18.95 | C |
| ATOM | 1182 | CG | ASN | A | 180 | 30.991 | −5.578 | 6.359 | 1.00 | 19.52 | C |
| ATOM | 1183 | OD1 | ASN | A | 180 | 30.594 | −6.649 | 6.784 | 1.00 | 18.65 | O |
| ATOM | 1184 | ND2 | ASN | A | 180 | 32.280 | −5.222 | 6.399 | 1.00 | 14.34 | N |
| ATOM | 1185 | C | ASN | A | 180 | 29.299 | −2.281 | 5.484 | 1.00 | 19.26 | C |
| ATOM | 1186 | O | ASN | A | 180 | 28.139 | −1.938 | 5.663 | 1.00 | 21.17 | O |
| ATOM | 1187 | N | VAL | A | 181 | 30.053 | −1.821 | 4.497 | 1.00 | 18.21 | N |
| ATOM | 1188 | CA | VAL | A | 181 | 29.474 | −1.282 | 3.266 | 1.00 | 16.97 | C |
| ATOM | 1189 | CB | VAL | A | 181 | 30.261 | −0.066 | 2.754 | 1.00 | 16.79 | C |
| ATOM | 1190 | CG1 | VAL | A | 181 | 30.147 | 1.098 | 3.727 | 1.00 | 18.35 | C |
| ATOM | 1191 | CG2 | VAL | A | 181 | 29.754 | 0.354 | 1.347 | 1.00 | 17.72 | C |
| ATOM | 1192 | C | VAL | A | 181 | 29.583 | −2.422 | 2.256 | 1.00 | 16.01 | C |
| ATOM | 1193 | O | VAL | A | 181 | 30.671 | −2.950 | 2.085 | 1.00 | 15.73 | O |
| ATOM | 1194 | N | CYS | A | 182 | 28.476 | −2.804 | 1.607 | 1.00 | 14.73 | N |
| ATOM | 1195 | CA | CYS | A | 182 | 28.505 | −3.824 | 0.540 | 1.00 | 15.14 | C |
| ATOM | 1196 | CB | CYS | A | 182 | 27.549 | −4.979 | 0.834 | 1.00 | 14.80 | C |
| ATOM | 1197 | SG | CYS | A | 182 | 27.981 | −5.808 | 2.314 | 1.00 | 17.69 | S |
| ATOM | 1198 | C | CYS | A | 182 | 28.147 | −3.255 | −0.809 | 1.00 | 15.03 | C |
| ATOM | 1199 | O | CYS | A | 182 | 27.392 | −2.274 | −0.910 | 1.00 | 15.86 | O |
| ATOM | 1200 | N | THR | A | 183 | 28.710 | −3.866 | −1.840 | 1.00 | 14.17 | N |
| ATOM | 1201 | CA | THR | A | 183 | 28.495 | −3.459 | −3.207 | 1.00 | 14.16 | C |
| ATOM | 1202 | CB | THR | A | 183 | 29.737 | −2.695 | −3.752 | 1.00 | 14.13 | C |
| ATOM | 1203 | OG1 | THR | A | 183 | 30.856 | −3.599 | −3.793 | 1.00 | 13.53 | O |
| ATOM | 1204 | CG2 | THR | A | 183 | 30.208 | −1.601 | −2.758 | 1.00 | 11.86 | C |
| ATOM | 1205 | C | THR | A | 183 | 28.223 | −4.674 | −4.080 | 1.00 | 15.18 | C |
| ATOM | 1206 | O | THR | A | 183 | 28.654 | −5.776 | −3.770 | 1.00 | 13.95 | O |
| ATOM | 1207 | N | LEU | A | 184 | 27.533 | −4.442 | −5.192 | 1.00 | 16.07 | N |
| ATOM | 1208 | CA | LEU | A | 184 | 27.212 | −5.502 | −6.123 | 1.00 | 16.80 | C |
| ATOM | 1209 | CB | LEU | A | 184 | 25.826 | −6.087 | −5.795 | 1.00 | 16.92 | C |
| ATOM | 1210 | CG | LEU | A | 184 | 25.320 | −7.347 | −6.512 | 1.00 | 15.17 | C |
| ATOM | 1211 | CD1 | LEU | A | 184 | 26.197 | −8.596 | −6.251 | 1.00 | 13.98 | C |
| ATOM | 1212 | CD2 | LEU | A | 184 | 23.835 | −7.575 | −6.067 | 1.00 | 17.86 | C |
| ATOM | 1213 | C | LEU | A | 184 | 27.158 | −4.940 | −7.513 | 1.00 | 17.50 | C |
| ATOM | 1214 | O | LEU | A | 184 | 26.643 | −3.831 | −7.722 | 1.00 | 17.44 | O |
| ATOM | 1215 | N | VAL | A | 185 | 27.675 | −5.703 | −8.468 | 1.00 | 17.39 | N |
| ATOM | 1216 | CA | VAL | A | 185 | 27.463 | −5.405 | −9.898 | 1.00 | 18.32 | C |
| ATOM | 1217 | CB | VAL | A | 185 | 28.698 | −5.800 | −10.711 | 1.00 | 18.11 | C |
| ATOM | 1218 | CG1 | VAL | A | 185 | 28.456 | −5.652 | −12.219 | 1.00 | 18.67 | C |
| ATOM | 1219 | CG2 | VAL | A | 185 | 29.913 | −4.963 | −10.273 | 1.00 | 17.71 | C |
| ATOM | 1220 | C | VAL | A | 185 | 26.287 | −6.270 | −10.344 | 1.00 | 19.82 | C |
| ATOM | 1221 | O | VAL | A | 185 | 26.307 | −7.476 | −10.174 | 1.00 | 18.99 | O |
| ATOM | 1222 | N | ARG | A | 186 | 25.254 | −5.654 | −10.901 | 1.00 | 21.85 | N |
| ATOM | 1223 | CA | ARG | A | 186 | 24.055 | −6.409 | −11.309 | 1.00 | 24.79 | C |
| ATOM | 1224 | CB | ARG | A | 186 | 22.790 | −5.580 | −11.004 | 1.00 | 25.59 | C |
| ATOM | 1225 | CG | ARG | A | 186 | 22.302 | −5.709 | −9.533 | 1.00 | 30.78 | C |
| ATOM | 1226 | CD | ARG | A | 186 | 21.133 | −4.825 | −9.225 | 1.00 | 39.49 | C |
| ATOM | 1227 | NE | ARG | A | 186 | 20.791 | −4.722 | −7.798 | 1.00 | 45.77 | N |
| ATOM | 1228 | CZ | ARG | A | 186 | 20.753 | −3.574 | −7.109 | 1.00 | 48.33 | C |
| ATOM | 1229 | NH1 | ARG | A | 186 | 21.089 | −2.414 | −7.690 | 1.00 | 49.62 | N |
| ATOM | 1230 | NH2 | ARG | A | 186 | 20.388 | −3.587 | −5.830 | 1.00 | 49.09 | N |
| ATOM | 1231 | C | ARG | A | 186 | 24.123 | −6.770 | −12.789 | 1.00 | 23.48 | C |
| ATOM | 1232 | O | ARG | A | 186 | 24.654 | −6.015 | −13.575 | 1.00 | 24.12 | O |
| ATOM | 1233 | N | GLY | A | 186A | 23.626 | −7.930 | −13.176 | 1.00 | 24.06 | N |
| ATOM | 1234 | CA | GLY | A | 186A | 23.543 | −8.252 | −14.602 | 1.00 | 23.85 | C |
| ATOM | 1235 | C | GLY | A | 186A | 24.766 | −8.907 | −15.236 | 1.00 | 23.88 | C |
| ATOM | 1236 | O | GLY | A | 186A | 24.820 | −9.149 | −16.464 | 1.00 | 24.36 | O |
| ATOM | 1237 | N | ARG | A | 187 | 25.780 | −9.155 | −14.409 | 1.00 | 22.41 | N |
| ATOM | 1238 | CA | ARG | A | 187 | 27.005 | −9.860 | −14.839 | 1.00 | 20.94 | C |
| ATOM | 1239 | CB | ARG | A | 187 | 27.839 | −9.027 | −15.823 | 1.00 | 20.23 | C |
| ATOM | 1240 | CG | ARG | A | 187 | 28.414 | −7.763 | −15.269 | 1.00 | 19.24 | C |
| ATOM | 1241 | CD | ARG | A | 187 | 28.835 | −6.705 | −16.337 | 1.00 | 21.56 | C |
| ATOM | 1242 | NE | ARG | A | 187 | 29.659 | −5.661 | −15.727 | 1.00 | 19.64 | N |
| ATOM | 1243 | CZ | ARG | A | 187 | 30.979 | −5.758 | −15.535 | 1.00 | 21.15 | C |
| ATOM | 1244 | NH1 | ARG | A | 187 | 31.656 | −6.825 | −15.946 | 1.00 | 15.69 | N |
| ATOM | 1245 | NH2 | ARG | A | 187 | 31.630 | −4.766 | −14.938 | 1.00 | 19.69 | N |
| ATOM | 1246 | C | ARG | A | 187 | 27.813 | −10.219 | −13.596 | 1.00 | 19.59 | C |
| ATOM | 1247 | O | ARG | A | 187 | 27.501 | −9.747 | −12.508 | 1.00 | 20.51 | O |
| ATOM | 1248 | N | GLN | A | 188 | 28.848 | −11.035 | −13.760 | 1.00 | 18.37 | N |
| ATOM | 1249 | CA | GLN | A | 188 | 29.696 | −11.464 | −12.636 | 1.00 | 16.71 | C |
| ATOM | 1250 | CB | GLN | A | 188 | 30.036 | −12.960 | −12.758 | 1.00 | 17.57 | C |
| ATOM | 1251 | CG | GLN | A | 188 | 28.888 | −13.902 | −12.311 | 1.00 | 17.51 | C |
| ATOM | 1252 | CD | GLN | A | 188 | 27.622 | −13.754 | −13.200 | 1.00 | 23.71 | C |
| ATOM | 1253 | OE1 | GLN | A | 188 | 27.577 | −14.251 | −14.340 | 1.00 | 26.75 | O |
| ATOM | 1254 | NE2 | GLN | A | 188 | 26.631 | −13.047 | −12.698 | 1.00 | 19.96 | N |
| ATOM | 1255 | C | GLN | A | 188 | 30.969 | −10.616 | −12.604 | 1.00 | 15.23 | C |
| ATOM | 1256 | O | GLN | A | 188 | 31.795 | −10.688 | −13.511 | 1.00 | 15.22 | O |
| ATOM | 1257 | N | ALA | A | 188A | 31.093 | −9.754 | −11.593 | 1.00 | 12.68 | N |
| ATOM | 1258 | CA | ALA | A | 188A | 32.248 | −8.866 | −11.512 | 1.00 | 12.20 | C |
| ATOM | 1259 | CB | ALA | A | 188A | 32.069 | −7.612 | −12.419 | 1.00 | 11.31 | C |
| ATOM | 1260 | C | ALA | A | 188A | 32.341 | −8.475 | −10.064 | 1.00 | 11.28 | C |
| ATOM | 1261 | O | ALA | A | 188A | 31.312 | −8.420 | −9.383 | 1.00 | 11.77 | O |
| ATOM | 1262 | N | GLY | A | 189 | 33.547 | −8.217 | −9.590 | 1.00 | 8.71 | N |
| ATOM | 1263 | CA | GLY | A | 189 | 33.743 | −7.819 | −8.209 | 1.00 | 9.93 | C |
| ATOM | 1264 | C | GLY | A | 189 | 35.217 | −7.816 | −7.865 | 1.00 | 10.38 | C |
| ATOM | 1265 | O | GLY | A | 189 | 36.063 | −8.015 | −8.761 | 1.00 | 10.70 | O |
| ATOM | 1266 | N | VAL | A | 190 | 35.513 | −7.623 | −6.579 | 1.00 | 9.02 | N |
| ATOM | 1267 | CA | VAL | A | 190 | 36.881 | −7.566 | −6.077 | 1.00 | 8.77 | C |
| ATOM | 1268 | CB | VAL | A | 190 | 37.017 | −6.757 | −4.750 | 1.00 | 7.99 | C |
| ATOM | 1269 | CG1 | VAL | A | 190 | 36.659 | −5.226 | −4.984 | 1.00 | 8.34 | C |
| ATOM | 1270 | CG2 | VAL | A | 190 | 36.139 | −7.393 | −3.584 | 1.00 | 8.14 | C |
| ATOM | 1271 | C | VAL | A | 190 | 37.308 | −9.018 | −5.824 | 1.00 | 10.14 | C |
| ATOM | 1272 | O | VAL | A | 190 | 36.474 | −9.944 | −5.825 | 1.00 | 9.63 | O |
| ATOM | 1273 | N | CYS | A | 191 | 38.606 | −9.209 | −5.637 | 1.00 | 9.83 | N |
| ATOM | 1274 | CA | CYS | A | 191 | 39.129 | −10.551 | −5.559 | 1.00 | 10.60 | C |
| ATOM | 1275 | CB | CYS | A | 191 | 39.274 | −11.172 | −6.972 | 1.00 | 11.34 | C |
| ATOM | 1276 | SG | CYS | A | 191 | 39.401 | −13.021 | −6.921 | 1.00 | 11.02 | S |
| ATOM | 1277 | C | CYS | A | 191 | 40.425 | −10.496 | −4.781 | 1.00 | 10.93 | C |
| ATOM | 1278 | O | CYS | A | 191 | 40.817 | −9.412 | −4.302 | 1.00 | 9.82 | O |
| ATOM | 1279 | N | PHE | A | 192 | 41.095 | −11.648 | −4.643 | 1.00 | 10.69 | N |
| ATOM | 1280 | CA | PHE | A | 192 | 42.329 | −11.715 | −3.865 | 1.00 | 11.21 | C |
| ATOM | 1281 | CB | PHE | A | 192 | 42.889 | −13.164 | −3.898 | 1.00 | 10.50 | C |
| ATOM | 1282 | CG | PHE | A | 192 | 41.995 | −14.153 | −3.224 | 1.00 | 11.52 | C |
| ATOM | 1283 | CD1 | PHE | A | 192 | 40.988 | −14.793 | −3.929 | 1.00 | 12.62 | C |
| ATOM | 1284 | CE1 | PHE | A | 192 | 40.119 | −15.710 | −3.296 | 1.00 | 13.98 | C |
| ATOM | 1285 | CZ | PHE | A | 192 | 40.286 | −16.009 | −1.934 | 1.00 | 13.49 | C |
| ATOM | 1286 | CE2 | PHE | A | 192 | 41.302 | −15.368 | −1.218 | 1.00 | 14.54 | C |
| ATOM | 1287 | CD2 | PHE | A | 192 | 42.144 | −14.430 | −1.874 | 1.00 | 13.15 | C |
| ATOM | 1288 | C | PHE | A | 192 | 43.365 | −10.728 | −4.371 | 1.00 | 10.89 | C |
| ATOM | 1289 | O | PHE | A | 192 | 43.552 | −10.605 | −5.590 | 1.00 | 11.50 | O |
| ATOM | 1290 | N | GLY | A | 193 | 44.067 | −10.065 | −3.448 | 1.00 | 11.08 | N |
| ATOM | 1291 | CA | GLY | A | 193 | 45.024 | −9.026 | −3.836 | 1.00 | 10.36 | C |
| ATOM | 1292 | C | GLY | A | 193 | 44.371 | −7.645 | −3.886 | 1.00 | 9.51 | C |
| ATOM | 1293 | O | GLY | A | 193 | 45.068 | −6.633 | −3.880 | 1.00 | 11.00 | O |
| ATOM | 1294 | N | ASP | A | 194 | 43.045 | −7.594 | −3.939 | 1.00 | 9.04 | N |
| ATOM | 1295 | CA | ASP | A | 194 | 42.289 | −6.338 | −3.812 | 1.00 | 8.76 | C |
| ATOM | 1296 | CB | ASP | A | 194 | 40.942 | −6.442 | −4.523 | 1.00 | 9.32 | C |
| ATOM | 1297 | CG | ASP | A | 194 | 41.081 | −6.502 | −6.024 | 1.00 | 11.10 | C |
| ATOM | 1298 | OD1 | ASP | A | 194 | 42.032 | −5.850 | −6.559 | 1.00 | 10.23 | O |
| ATOM | 1299 | OD2 | ASP | A | 194 | 40.304 | −7.215 | −6.715 | 1.00 | 10.66 | O |
| ATOM | 1300 | C | ASP | A | 194 | 42.027 | −5.931 | −2.371 | 1.00 | 9.75 | C |
| ATOM | 1301 | O | ASP | A | 194 | 41.666 | −4.770 | −2.127 | 1.00 | 9.98 | O |
| ATOM | 1302 | N | SER | A | 195 | 42.171 | −6.869 | −1.423 | 1.00 | 9.74 | N |
| ATOM | 1303 | CA | SER | A | 195 | 42.026 | −6.579 | 0.013 | 1.00 | 10.92 | C |
| ATOM | 1304 | CB | SER | A | 195 | 42.676 | −7.679 | 0.838 | 1.00 | 11.61 | C |
| ATOM | 1305 | OG | SER | A | 195 | 42.055 | −8.934 | 0.594 | 1.00 | 15.37 | O |
| ATOM | 1306 | C | SER | A | 195 | 42.746 | −5.293 | 0.337 | 1.00 | 10.78 | C |
| ATOM | 1307 | O | SER | A | 195 | 43.877 | −5.068 | −0.157 | 1.00 | 10.75 | O |
| ATOM | 1308 | N | GLY | A | 196 | 42.091 | −4.434 | 1.116 | 1.00 | 11.01 | N |
| ATOM | 1309 | CA | GLY | A | 196 | 42.761 | −3.248 | 1.638 | 1.00 | 10.54 | C |
| ATOM | 1310 | C | GLY | A | 196 | 42.573 | −2.056 | 0.712 | 1.00 | 11.17 | C |
| ATOM | 1311 | O | GLY | A | 196 | 42.909 | −0.937 | 1.106 | 1.00 | 11.09 | O |
| ATOM | 1312 | N | SER | A | 197 | 42.073 | −2.293 | −0.507 | 1.00 | 10.37 | N |
| ATOM | 1313 | CA | SER | A | 197 | 41.974 | −1.212 | −1.515 | 1.00 | 10.55 | C |
| ATOM | 1314 | CB | SER | A | 197 | 41.852 | −1.771 | −2.934 | 1.00 | 10.91 | C |
| ATOM | 1315 | OG | SER | A | 197 | 42.894 | −2.618 | −3.257 | 1.00 | 10.79 | O |
| ATOM | 1316 | C | SER | A | 197 | 40.765 | −0.318 | −1.232 | 1.00 | 9.90 | C |
| ATOM | 1317 | O | SER | A | 197 | 39.758 | −0.781 | −0.670 | 1.00 | 9.50 | O |
| ATOM | 1318 | N | PRO | A | 198 | 40.846 | 0.949 | −1.636 | 1.00 | 9.77 | N |
| ATOM | 1319 | CA | PRO | A | 198 | 39.742 | 1.888 | −1.431 | 1.00 | 10.21 | C |
| ATOM | 1320 | CB | PRO | A | 198 | 40.343 | 3.242 | −1.905 | 1.00 | 10.19 | C |
| ATOM | 1321 | CG | PRO | A | 198 | 41.371 | 2.809 | −2.934 | 1.00 | 9.39 | C |
| ATOM | 1322 | CD | PRO | A | 198 | 42.023 | 1.626 | −2.220 | 1.00 | 9.39 | C |
| ATOM | 1323 | C | PRO | A | 198 | 38.473 | 1.594 | −2.241 | 1.00 | 9.78 | C |
| ATOM | 1324 | O | PRO | A | 198 | 38.534 | 1.102 | −3.382 | 1.00 | 11.16 | O |
| ATOM | 1325 | N | LEU | A | 199 | 37.333 | 1.918 | −1.642 | 1.00 | 10.60 | N |
| ATOM | 1326 | CA | LEU | A | 199 | 36.103 | 2.028 | −2.372 | 1.00 | 10.51 | C |
| ATOM | 1327 | CB | LEU | A | 199 | 35.000 | 1.229 | −1.691 | 1.00 | 10.03 | C |
| ATOM | 1328 | CG | LEU | A | 199 | 33.579 | 1.294 | −2.268 | 1.00 | 10.65 | C |
| ATOM | 1329 | CD1 | LEU | A | 199 | 33.401 | 0.484 | −3.582 | 1.00 | 9.51 | C |
| ATOM | 1330 | CD2 | LEU | A | 199 | 32.557 | 0.851 | −1.218 | 1.00 | 12.49 | C |
| ATOM | 1331 | C | LEU | A | 199 | 35.762 | 3.523 | −2.411 | 1.00 | 12.06 | C |
| ATOM | 1332 | O | LEU | A | 199 | 35.632 | 4.156 | −1.345 | 1.00 | 11.39 | O |
| ATOM | 1333 | N | VAL | A | 200 | 35.630 | 4.089 | −3.625 | 1.00 | 12.47 | N |
| ATOM | 1334 | CA | VAL | A | 200 | 35.451 | 5.550 | −3.773 | 1.00 | 12.99 | C |
| ATOM | 1335 | CB | VAL | A | 200 | 36.347 | 6.139 | −4.909 | 1.00 | 12.94 | C |
| ATOM | 1336 | CG1 | VAL | A | 200 | 36.309 | 7.724 | −4.926 | 1.00 | 11.93 | C |
| ATOM | 1337 | CG2 | VAL | A | 200 | 37.807 | 5.676 | −4.759 | 1.00 | 12.06 | C |
| ATOM | 1338 | C | VAL | A | 200 | 33.993 | 5.822 | −4.087 | 1.00 | 13.88 | C |
| ATOM | 1339 | O | VAL | A | 200 | 33.487 | 5.332 | −5.076 | 1.00 | 13.01 | O |
| ATOM | 1340 | N | CYS | A | 201 | 33.311 | 6.595 | −3.239 | 1.00 | 14.64 | N |
| ATOM | 1341 | CA | CYS | A | 201 | 31.903 | 6.896 | −3.478 | 1.00 | 14.74 | C |
| ATOM | 1342 | CB | CYS | A | 201 | 31.002 | 6.167 | −2.477 | 1.00 | 14.24 | C |
| ATOM | 1343 | SG | CYS | A | 201 | 31.246 | 4.372 | −2.332 | 1.00 | 14.29 | S |
| ATOM | 1344 | C | CYS | A | 201 | 31.727 | 8.401 | −3.317 | 1.00 | 15.05 | C |
| ATOM | 1345 | O | CYS | A | 201 | 32.129 | 8.983 | −2.293 | 1.00 | 13.38 | O |
| ATOM | 1346 | N | ASN | A | 204 | 31.194 | 9.023 | −4.368 | 1.00 | 15.30 | N |
| ATOM | 1347 | CA | ASN | A | 204 | 30.951 | 10.452 | −4.368 | 1.00 | 15.89 | C |
| ATOM | 1348 | CB | ASN | A | 204 | 29.831 | 10.788 | −3.366 | 1.00 | 17.20 | C |
| ATOM | 1349 | CG | ASN | A | 204 | 28.494 | 10.005 | −3.631 | 1.00 | 19.92 | C |
| ATOM | 1350 | OD1 | ASN | A | 204 | 27.851 | 9.507 | −2.693 | 1.00 | 24.31 | O |
| ATOM | 1351 | ND2 | ASN | A | 204 | 28.097 | 9.906 | −4.897 | 1.00 | 22.60 | N |
| ATOM | 1352 | C | ASN | A | 204 | 32.250 | 11.174 | −4.011 | 1.00 | 16.22 | C |
| ATOM | 1353 | O | ASN | A | 204 | 32.238 | 12.078 | −3.189 | 1.00 | 16.28 | O |
| ATOM | 1354 | N | GLY | A | 205 | 33.376 | 10.696 | −4.549 | 1.00 | 16.06 | N |
| ATOM | 1355 | CA | GLY | A | 205 | 34.678 | 11.329 | −4.363 | 1.00 | 16.08 | C |
| ATOM | 1356 | C | GLY | A | 205 | 35.379 | 11.051 | −3.033 | 1.00 | 16.27 | C |
| ATOM | 1357 | O | GLY | A | 205 | 36.494 | 11.527 | −2.776 | 1.00 | 17.83 | O |
| ATOM | 1358 | N | LEU | A | 208 | 34.768 | 10.240 | −2.191 | 1.00 | 15.47 | N |
| ATOM | 1359 | CA | LEU | A | 208 | 35.269 | 10.054 | −0.834 | 1.00 | 14.47 | C |
| ATOM | 1360 | CB | LEU | A | 208 | 34.162 | 10.450 | 0.137 | 1.00 | 14.64 | C |
| ATOM | 1361 | CG | LEU | A | 208 | 34.030 | 11.856 | 0.741 | 1.00 | 17.03 | C |
| ATOM | 1362 | CD1 | LEU | A | 208 | 34.881 | 12.930 | 0.142 | 1.00 | 14.60 | C |
| ATOM | 1363 | CD2 | LEU | A | 208 | 32.608 | 12.314 | 1.130 | 1.00 | 15.06 | C |
| ATOM | 1364 | C | LEU | A | 208 | 35.586 | 8.563 | −0.607 | 1.00 | 14.40 | C |
| ATOM | 1365 | O | LEU | A | 208 | 34.907 | 7.688 | −1.196 | 1.00 | 14.29 | O |
| ATOM | 1366 | N | ILE | A | 209 | 36.576 | 8.259 | 0.230 | 1.00 | 12.72 | N |
| ATOM | 1367 | CA | ILE | A | 209 | 36.909 | 6.839 | 0.456 | 1.00 | 12.71 | C |
| ATOM | 1368 | CB | ILE | A | 209 | 38.331 | 6.609 | 0.941 | 1.00 | 12.88 | C |
| ATOM | 1369 | CG1 | ILE | A | 209 | 39.318 | 7.353 | 0.055 | 1.00 | 11.36 | C |
| ATOM | 1370 | CD1 | ILE | A | 209 | 39.131 | 7.141 | −1.469 | 1.00 | 10.98 | C |
| ATOM | 1371 | CG2 | ILE | A | 209 | 38.685 | 5.052 | 0.941 | 1.00 | 11.67 | C |
| ATOM | 1372 | C | ILE | A | 209 | 35.945 | 6.248 | 1.442 | 1.00 | 13.21 | C |
| ATOM | 1373 | O | ILE | A | 209 | 36.074 | 6.470 | 2.637 | 1.00 | 14.27 | O |
| ATOM | 1374 | N | HIS | A | 210 | 34.957 | 5.513 | 0.966 | 1.00 | 12.42 | N |
| ATOM | 1375 | CA | HIS | A | 210 | 33.967 | 5.036 | 1.916 | 1.00 | 13.34 | C |
| ATOM | 1376 | CB | HIS | A | 210 | 32.609 | 5.185 | 1.310 | 1.00 | 13.29 | C |
| ATOM | 1377 | CG | HIS | A | 210 | 31.898 | 6.405 | 1.768 | 1.00 | 13.93 | C |
| ATOM | 1378 | ND1 | HIS | A | 210 | 31.811 | 7.547 | 0.999 | 1.00 | 18.86 | N |
| ATOM | 1379 | CE1 | HIS | A | 210 | 31.085 | 8.446 | 1.642 | 1.00 | 14.86 | C |
| ATOM | 1380 | NE2 | HIS | A | 210 | 30.724 | 7.932 | 2.805 | 1.00 | 18.05 | N |
| ATOM | 1381 | CD2 | HIS | A | 210 | 31.214 | 6.654 | 2.903 | 1.00 | 12.01 | C |
| ATOM | 1382 | C | HIS | A | 210 | 34.155 | 3.594 | 2.375 | 1.00 | 13.26 | C |
| ATOM | 1383 | O | HIS | A | 210 | 33.480 | 3.132 | 3.295 | 1.00 | 13.15 | O |
| ATOM | 1384 | N | GLY | A | 211 | 35.058 | 2.880 | 1.722 | 1.00 | 12.12 | N |
| ATOM | 1385 | CA | GLY | A | 211 | 35.221 | 1.476 | 2.036 | 1.00 | 11.24 | C |
| ATOM | 1386 | C | GLY | A | 211 | 36.669 | 1.088 | 1.883 | 1.00 | 11.55 | C |
| ATOM | 1387 | O | GLY | A | 211 | 37.457 | 1.758 | 1.162 | 1.00 | 11.92 | O |
| ATOM | 1388 | N | ILE | A | 212 | 37.025 | 0.024 | 2.593 | 1.00 | 11.54 | N |
| ATOM | 1389 | CA | ILE | A | 212 | 38.288 | −0.693 | 2.430 | 1.00 | 11.93 | C |
| ATOM | 1390 | CB | ILE | A | 212 | 39.080 | −0.664 | 3.776 | 1.00 | 12.86 | C |
| ATOM | 1391 | CG1 | ILE | A | 212 | 39.465 | 0.781 | 4.138 | 1.00 | 13.38 | C |
| ATOM | 1392 | CD1 | ILE | A | 212 | 39.871 | 0.946 | 5.634 | 1.00 | 16.74 | C |
| ATOM | 1393 | CG2 | ILE | A | 212 | 40.360 | −1.602 | 3.723 | 1.00 | 12.00 | C |
| ATOM | 1394 | C | ILE | A | 212 | 37.909 | −2.133 | 2.134 | 1.00 | 11.81 | C |
| ATOM | 1395 | O | ILE | A | 212 | 37.181 | −2.738 | 2.908 | 1.00 | 10.99 | O |
| ATOM | 1396 | N | ALA | A | 213 | 38.420 | −2.700 | 1.051 | 1.00 | 11.42 | N |
| ATOM | 1397 | CA | ALA | A | 213 | 38.002 | −4.052 | 0.638 | 1.00 | 11.38 | C |
| ATOM | 1398 | CB | ALA | A | 213 | 38.573 | −4.447 | −0.770 | 1.00 | 9.46 | C |
| ATOM | 1399 | C | ALA | A | 213 | 38.316 | −5.095 | 1.703 | 1.00 | 10.31 | C |
| ATOM | 1400 | O | ALA | A | 213 | 39.460 | −5.209 | 2.160 | 1.00 | 10.70 | O |
| ATOM | 1401 | N | SER | A | 214 | 37.279 | −5.826 | 2.127 | 1.00 | 10.46 | N |
| ATOM | 1402 | CA | SER | A | 214 | 37.378 | −6.682 | 3.312 | 1.00 | 10.15 | C |
| ATOM | 1403 | CB | SER | A | 214 | 36.462 | −6.207 | 4.486 | 1.00 | 10.35 | C |
| ATOM | 1404 | OG | SER | A | 214 | 36.504 | −7.096 | 5.645 | 1.00 | 11.22 | O |
| ATOM | 1405 | C | SER | A | 214 | 37.124 | −8.144 | 2.945 | 1.00 | 11.66 | C |
| ATOM | 1406 | O | SER | A | 214 | 38.010 | −8.954 | 3.150 | 1.00 | 11.23 | O |
| ATOM | 1407 | N | PHE | A | 215 | 35.962 | −8.500 | 2.380 | 1.00 | 11.21 | N |
| ATOM | 1408 | CA | PHE | A | 215 | 35.777 | −9.950 | 2.074 | 1.00 | 12.11 | C |
| ATOM | 1409 | CB | PHE | A | 215 | 35.527 | −10.789 | 3.360 | 1.00 | 11.29 | C |
| ATOM | 1410 | CG | PHE | A | 215 | 34.244 | −10.436 | 4.088 | 1.00 | 14.12 | C |
| ATOM | 1411 | CD1 | PHE | A | 215 | 34.249 | −9.469 | 5.112 | 1.00 | 13.73 | C |
| ATOM | 1412 | CE1 | PHE | A | 215 | 33.040 | −9.122 | 5.782 | 1.00 | 15.87 | C |
| ATOM | 1413 | CZ | PHE | A | 215 | 31.821 | −9.764 | 5.430 | 1.00 | 17.18 | C |
| ATOM | 1414 | CE2 | PHE | A | 215 | 31.819 | −10.719 | 4.399 | 1.00 | 18.13 | C |
| ATOM | 1415 | CD2 | PHE | A | 215 | 33.034 | −11.050 | 3.744 | 1.00 | 13.65 | C |
| ATOM | 1416 | C | PHE | A | 215 | 34.739 | −10.237 | 1.016 | 1.00 | 11.58 | C |
| ATOM | 1417 | O | PHE | A | 215 | 33.834 | −9.453 | 0.811 | 1.00 | 11.55 | O |
| ATOM | 1418 | N | VAL | A | 216 | 34.885 | −11.374 | 0.332 | 1.00 | 13.05 | N |
| ATOM | 1419 | CA | VAL | A | 216 | 33.888 | −11.851 | −0.625 | 1.00 | 13.01 | C |
| ATOM | 1420 | CB | VAL | A | 216 | 34.510 | −12.102 | −2.036 | 1.00 | 13.37 | C |
| ATOM | 1421 | CG1 | VAL | A | 216 | 35.210 | −10.863 | −2.564 | 1.00 | 12.15 | C |
| ATOM | 1422 | CG2 | VAL | A | 216 | 35.473 | −13.316 | −2.015 | 1.00 | 12.42 | C |
| ATOM | 1423 | C | VAL | A | 216 | 33.190 | −13.154 | −0.096 | 1.00 | 13.97 | C |
| ATOM | 1424 | O | VAL | A | 216 | 33.659 | −13.792 | 0.830 | 1.00 | 14.98 | O |
| ATOM | 1425 | N | ARG | A | 217 | 32.035 | −13.500 | −0.629 | 1.00 | 14.44 | N |
| ATOM | 1426 | CA | ARG | A | 217 | 31.400 | −14.765 | −0.274 | 1.00 | 16.04 | C |
| ATOM | 1427 | CB | ARG | A | 217 | 30.107 | −14.578 | 0.560 | 1.00 | 16.20 | C |
| ATOM | 1428 | CG | ARG | A | 217 | 30.327 | −14.042 | 2.010 | 1.00 | 18.92 | C |
| ATOM | 1429 | CD | ARG | A | 217 | 29.073 | −13.347 | 2.586 | 1.00 | 19.54 | C |
| ATOM | 1430 | NE | ARG | A | 217 | 28.901 | −11.993 | 2.068 | 1.00 | 20.52 | N |
| ATOM | 1431 | CZ | ARG | A | 217 | 27.761 | −11.295 | 2.063 | 1.00 | 24.71 | C |
| ATOM | 1432 | NH1 | ARG | A | 217 | 26.612 | −11.796 | 2.554 | 1.00 | 23.73 | N |
| ATOM | 1433 | NH2 | ARG | A | 217 | 27.761 | −10.076 | 1.559 | 1.00 | 24.19 | N |
| ATOM | 1434 | C | ARG | A | 217 | 31.036 | −15.418 | −1.594 | 1.00 | 16.08 | C |
| ATOM | 1435 | O | ARG | A | 217 | 30.720 | −14.706 | −2.576 | 1.00 | 15.42 | O |
| ATOM | 1436 | N | GLY | A | 218 | 31.057 | −16.750 | −1.608 | 1.00 | 15.60 | N |
| ATOM | 1437 | CA | GLY | A | 218 | 30.789 | −17.505 | −2.813 | 1.00 | 15.83 | C |
| ATOM | 1438 | C | GLY | A | 218 | 31.897 | −17.410 | −3.863 | 1.00 | 15.44 | C |
| ATOM | 1439 | O | GLY | A | 218 | 31.639 | −17.627 | −5.043 | 1.00 | 13.87 | O |
| ATOM | 1440 | N | GLY | A | 219 | 33.117 | −17.110 | −3.419 | 1.00 | 15.03 | N |
| ATOM | 1441 | CA | GLY | A | 219 | 34.237 | −16.864 | −4.325 | 1.00 | 14.41 | C |
| ATOM | 1442 | C | GLY | A | 219 | 34.104 | −15.472 | −4.922 | 1.00 | 14.53 | C |
| ATOM | 1443 | O | GLY | A | 219 | 33.075 | −14.807 | −4.716 | 1.00 | 14.72 | O |
| ATOM | 1444 | N | CYS | A | 220 | 35.086 | −15.045 | −5.726 | 1.00 | 13.40 | N |
| ATOM | 1445 | CA | CYS | A | 220 | 35.031 | −13.713 | −6.304 | 1.00 | 12.40 | C |
| ATOM | 1446 | CB | CYS | A | 220 | 36.390 | −13.400 | −6.923 | 1.00 | 12.34 | C |
| ATOM | 1447 | SG | CYS | A | 220 | 37.786 | −13.594 | −5.815 | 1.00 | 14.23 | S |
| ATOM | 1448 | C | CYS | A | 220 | 33.980 | −13.712 | −7.400 | 1.00 | 12.56 | C |
| ATOM | 1449 | O | CYS | A | 220 | 33.816 | −14.728 | −8.082 | 1.00 | 13.21 | O |
| ATOM | 1450 | N | ALA | A | 221 | 33.316 | −12.567 | −7.589 | 1.00 | 11.12 | N |
| ATOM | 1451 | CA | ALA | A | 221 | 32.435 | −12.299 | −8.706 | 1.00 | 12.06 | C |
| ATOM | 1452 | CB | ALA | A | 221 | 33.243 | −12.063 | −10.001 | 1.00 | 9.99 | C |
| ATOM | 1453 | C | ALA | A | 221 | 31.421 | −13.444 | −8.835 | 1.00 | 13.28 | C |
| ATOM | 1454 | O | ALA | A | 221 | 31.200 | −13.979 | −9.926 | 1.00 | 13.29 | O |
| ATOM | 1455 | N | SER | A | 222 | 30.792 | −13.804 | −7.703 | 1.00 | 13.31 | N |
| ATOM | 1456 | CA | SER | A | 222 | 29.766 | −14.847 | −7.664 | 1.00 | 14.87 | C |
| ATOM | 1457 | CB | SER | A | 222 | 29.381 | −15.153 | −6.196 | 1.00 | 15.14 | C |
| ATOM | 1458 | OG | SER | A | 222 | 28.734 | −14.046 | −5.594 | 1.00 | 14.44 | O |
| ATOM | 1459 | C | SER | A | 222 | 28.513 | −14.466 | −8.439 | 1.00 | 15.79 | C |
| ATOM | 1460 | O | SER | A | 222 | 27.822 | −15.331 | −8.981 | 1.00 | 16.17 | O |
| ATOM | 1461 | N | GLY | A | 222A | 28.213 | −13.165 | −8.471 | 1.00 | 16.39 | N |
| ATOM | 1462 | CA | GLY | A | 222A | 26.967 | −12.679 | −9.020 | 1.00 | 16.79 | C |
| ATOM | 1463 | C | GLY | A | 222A | 25.832 | −12.974 | −8.053 | 1.00 | 17.49 | C |
| ATOM | 1464 | O | GLY | A | 222A | 24.663 | −12.978 | −8.430 | 1.00 | 19.11 | O |
| ATOM | 1465 | N | LEU | A | 223 | 26.156 | −13.257 | −6.809 | 1.00 | 17.21 | N |
| ATOM | 1466 | CA | LEU | A | 223 | 25.098 | −13.555 | −5.846 | 1.00 | 17.26 | C |
| ATOM | 1467 | CB | LEU | A | 223 | 24.991 | −15.068 | −5.595 | 1.00 | 16.66 | C |
| ATOM | 1468 | CG | LEU | A | 223 | 24.156 | −15.527 | −4.366 | 1.00 | 17.18 | C |
| ATOM | 1469 | CD1 | LEU | A | 223 | 22.655 | −15.102 | −4.532 | 1.00 | 17.14 | C |
| ATOM | 1470 | CD2 | LEU | A | 223 | 24.265 | −17.028 | −4.181 | 1.00 | 14.39 | C |
| ATOM | 1471 | C | LEU | A | 223 | 25.303 | −12.766 | −4.545 | 1.00 | 17.21 | C |
| ATOM | 1472 | O | LEU | A | 223 | 24.416 | −12.021 | −4.134 | 1.00 | 17.94 | O |
| ATOM | 1473 | N | TYR | A | 224 | 26.459 | −12.939 | −3.912 | 1.00 | 16.26 | N |
| ATOM | 1474 | CA | TYR | A | 224 | 26.816 | −12.199 | −2.714 | 1.00 | 16.54 | C |
| ATOM | 1475 | CB | TYR | A | 224 | 27.711 | −13.028 | −1.791 | 1.00 | 15.77 | C |
| ATOM | 1476 | CG | TYR | A | 224 | 27.110 | −14.368 | −1.515 | 1.00 | 18.79 | C |
| ATOM | 1477 | CD1 | TYR | A | 224 | 25.976 | −14.492 | −0.666 | 1.00 | 20.73 | C |
| ATOM | 1478 | CE1 | TYR | A | 224 | 25.373 | −15.731 | −0.451 | 1.00 | 23.69 | C |
| ATOM | 1479 | CZ | TYR | A | 224 | 25.920 | −16.869 | −1.073 | 1.00 | 24.90 | C |
| ATOM | 1480 | OH | TYR | A | 224 | 25.359 | −18.107 | −0.861 | 1.00 | 28.74 | O |
| ATOM | 1481 | CE2 | TYR | A | 224 | 27.028 | −16.769 | −1.921 | 1.00 | 21.94 | C |
| ATOM | 1482 | CD2 | TYR | A | 224 | 27.609 | −15.505 | −2.144 | 1.00 | 16.52 | C |
| ATOM | 1483 | C | TYR | A | 224 | 27.512 | −10.872 | −3.031 | 1.00 | 16.24 | C |
| ATOM | 1484 | O | TYR | A | 224 | 28.482 | −10.829 | −3.773 | 1.00 | 15.88 | O |
| ATOM | 1485 | N | PRO | A | 225 | 27.037 | −9.797 | −2.416 | 1.00 | 16.65 | N |
| ATOM | 1486 | CA | PRO | A | 225 | 27.743 | −8.500 | −2.488 | 1.00 | 14.95 | C |
| ATOM | 1487 | CB | PRO | A | 225 | 26.852 | −7.574 | −1.667 | 1.00 | 15.37 | C |
| ATOM | 1488 | CG | PRO | A | 225 | 25.455 | −8.224 | −1.787 | 1.00 | 18.10 | C |
| ATOM | 1489 | CD | PRO | A | 225 | 25.794 | −9.725 | −1.618 | 1.00 | 17.21 | C |
| ATOM | 1490 | C | PRO | A | 225 | 29.141 | −8.588 | −1.878 | 1.00 | 14.71 | C |
| ATOM | 1491 | O | PRO | A | 225 | 29.374 | −9.375 | −0.948 | 1.00 | 14.51 | O |
| ATOM | 1492 | N | ASP | A | 226 | 30.070 | −7.788 | −2.394 | 1.00 | 13.36 | N |
| ATOM | 1493 | CA | ASP | A | 226 | 31.369 | −7.685 | −1.765 | 1.00 | 13.51 | C |
| ATOM | 1494 | CB | ASP | A | 226 | 32.384 | −7.068 | −2.715 | 1.00 | 12.53 | C |
| ATOM | 1495 | CG | ASP | A | 226 | 32.591 | −7.901 | −3.967 | 1.00 | 13.98 | C |
| ATOM | 1496 | OD1 | ASP | A | 226 | 33.014 | −7.322 | −4.987 | 1.00 | 12.56 | O |
| ATOM | 1497 | OD2 | ASP | A | 226 | 32.352 | −9.132 | −4.026 | 1.00 | 13.32 | O |
| ATOM | 1498 | C | ASP | A | 226 | 31.225 | −6.796 | −0.529 | 1.00 | 12.93 | C |
| ATOM | 1499 | O | ASP | A | 226 | 30.355 | −5.906 | −0.485 | 1.00 | 14.13 | O |
| ATOM | 1500 | N | ALA | A | 227 | 32.092 | −7.029 | 0.438 | 1.00 | 12.12 | N |
| ATOM | 1501 | CA | ALA | A | 227 | 32.031 | −6.373 | 1.725 | 1.00 | 13.02 | C |
| ATOM | 1502 | CB | ALA | A | 227 | 31.927 | −7.429 | 2.824 | 1.00 | 12.45 | C |
| ATOM | 1503 | C | ALA | A | 227 | 33.279 | −5.539 | 1.922 | 1.00 | 13.41 | C |
| ATOM | 1504 | O | ALA | A | 227 | 34.394 | −5.996 | 1.654 | 1.00 | 13.89 | O |
| ATOM | 1505 | N | PHE | A | 228 | 33.081 | −4.322 | 2.409 | 1.00 | 13.36 | N |
| ATOM | 1506 | CA | PHE | A | 228 | 34.162 | −3.371 | 2.646 | 1.00 | 13.72 | C |
| ATOM | 1507 | CB | PHE | A | 228 | 33.957 | −2.166 | 1.719 | 1.00 | 13.24 | C |
| ATOM | 1508 | CG | PHE | A | 228 | 34.157 | −2.469 | 0.268 | 1.00 | 14.45 | C |
| ATOM | 1509 | CD1 | PHE | A | 228 | 35.400 | −2.204 | −0.344 | 1.00 | 10.34 | C |
| ATOM | 1510 | CE1 | PHE | A | 228 | 35.601 | −2.497 | −1.706 | 1.00 | 10.48 | C |
| ATOM | 1511 | CZ | PHE | A | 228 | 34.549 | −3.064 | −2.472 | 1.00 | 9.65 | C |
| ATOM | 1512 | CE2 | PHE | A | 228 | 33.300 | −3.304 | −1.886 | 1.00 | 11.21 | C |
| ATOM | 1513 | CD2 | PHE | A | 228 | 33.099 | −3.004 | −0.518 | 1.00 | 12.75 | C |
| ATOM | 1514 | C | PHE | A | 228 | 34.106 | −2.927 | 4.131 | 1.00 | 14.06 | C |
| ATOM | 1515 | O | PHE | A | 228 | 33.020 | −2.790 | 4.683 | 1.00 | 14.76 | O |
| ATOM | 1516 | N | ALA | A | 229 | 35.241 | −2.723 | 4.793 | 1.00 | 13.84 | N |
| ATOM | 1517 | CA | ALA | A | 229 | 35.194 | −1.983 | 6.082 | 1.00 | 14.33 | C |
| ATOM | 1518 | CB | ALA | A | 229 | 36.601 | −1.891 | 6.709 | 1.00 | 13.58 | C |
| ATOM | 1519 | C | ALA | A | 229 | 34.576 | −0.553 | 5.881 | 1.00 | 14.63 | C |
| ATOM | 1520 | O | ALA | A | 229 | 34.933 | 0.137 | 4.925 | 1.00 | 15.20 | O |
| ATOM | 1521 | N | PRO | A | 230 | 33.618 | −0.137 | 6.705 | 1.00 | 14.98 | N |
| ATOM | 1522 | CA | PRO | A | 230 | 32.960 | 1.152 | 6.488 | 1.00 | 15.54 | C |
| ATOM | 1523 | CB | PRO | A | 230 | 31.639 | 1.014 | 7.270 | 1.00 | 15.67 | C |
| ATOM | 1524 | CG | PRO | A | 230 | 31.964 | 0.038 | 8.385 | 1.00 | 15.04 | C |
| ATOM | 1525 | CD | PRO | A | 230 | 33.017 | −0.884 | 7.834 | 1.00 | 14.73 | C |
| ATOM | 1526 | C | PRO | A | 230 | 33.792 | 2.298 | 7.047 | 1.00 | 16.10 | C |
| ATOM | 1527 | O | PRO | A | 230 | 33.731 | 2.564 | 8.266 | 1.00 | 17.43 | O |
| ATOM | 1528 | N | VAL | A | 231 | 34.540 | 2.962 | 6.176 | 1.00 | 15.04 | N |
| ATOM | 1529 | CA | VAL | A | 231 | 35.522 | 3.961 | 6.593 | 1.00 | 14.66 | C |
| ATOM | 1530 | CB | VAL | A | 231 | 36.215 | 4.617 | 5.389 | 1.00 | 14.95 | C |
| ATOM | 1531 | CG1 | VAL | A | 231 | 37.012 | 3.551 | 4.584 | 1.00 | 12.02 | C |
| ATOM | 1532 | CG2 | VAL | A | 231 | 37.139 | 5.777 | 5.840 | 1.00 | 12.07 | C |
| ATOM | 1533 | C | VAL | A | 231 | 34.856 | 5.032 | 7.456 | 1.00 | 15.76 | C |
| ATOM | 1534 | O | VAL | A | 231 | 35.440 | 5.480 | 8.453 | 1.00 | 15.61 | O |
| ATOM | 1535 | N | ALA | A | 232 | 33.631 | 5.409 | 7.097 | 1.00 | 15.53 | N |
| ATOM | 1536 | CA | ALA | A | 232 | 32.950 | 6.477 | 7.815 | 1.00 | 16.21 | C |
| ATOM | 1537 | CB | ALA | A | 232 | 31.648 | 6.812 | 7.162 | 1.00 | 16.65 | C |
| ATOM | 1538 | C | ALA | A | 232 | 32.744 | 6.175 | 9.308 | 1.00 | 17.45 | C |
| ATOM | 1539 | O | ALA | A | 232 | 32.664 | 7.111 | 10.110 | 1.00 | 18.04 | O |
| ATOM | 1540 | N | GLN | A | 233 | 32.661 | 4.887 | 9.675 | 1.00 | 18.74 | N |
| ATOM | 1541 | CA | GLN | A | 233 | 32.509 | 4.476 | 11.071 | 1.00 | 20.23 | C |
| ATOM | 1542 | CB | GLN | A | 233 | 32.123 | 2.986 | 11.194 | 1.00 | 21.35 | C |
| ATOM | 1543 | CG | GLN | A | 233 | 30.677 | 2.651 | 10.740 | 1.00 | 24.94 | C |
| ATOM | 1544 | CD | GLN | A | 233 | 30.277 | 1.200 | 11.036 | 1.00 | 32.16 | C |
| ATOM | 1545 | OE1 | GLN | A | 233 | 30.860 | 0.549 | 11.948 | 1.00 | 33.30 | O |
| ATOM | 1546 | NE2 | GLN | A | 233 | 29.288 | 0.679 | 10.273 | 1.00 | 31.33 | N |
| ATOM | 1547 | C | GLN | A | 233 | 33.789 | 4.715 | 11.861 | 1.00 | 19.80 | C |
| ATOM | 1548 | O | GLN | A | 233 | 33.767 | 4.739 | 13.111 | 1.00 | 21.20 | O |
| ATOM | 1549 | N | PHE | A | 234 | 34.903 | 4.905 | 11.164 | 1.00 | 18.33 | N |
| ATOM | 1550 | CA | PHE | A | 234 | 36.179 | 4.980 | 11.871 | 1.00 | 17.42 | C |
| ATOM | 1551 | CB | PHE | A | 234 | 37.136 | 3.878 | 11.398 | 1.00 | 17.31 | C |
| ATOM | 1552 | CG | PHE | A | 234 | 36.541 | 2.514 | 11.513 | 1.00 | 19.80 | C |
| ATOM | 1553 | CD1 | PHE | A | 234 | 36.130 | 1.815 | 10.370 | 1.00 | 19.12 | C |
| ATOM | 1554 | CE1 | PHE | A | 234 | 35.562 | 0.560 | 10.470 | 1.00 | 20.22 | C |
| ATOM | 1555 | CZ | PHE | A | 234 | 35.352 | −0.008 | 11.740 | 1.00 | 18.97 | C |
| ATOM | 1556 | CE2 | PHE | A | 234 | 35.753 | 0.689 | 12.881 | 1.00 | 20.25 | C |
| ATOM | 1557 | CD2 | PHE | A | 234 | 36.340 | 1.946 | 12.760 | 1.00 | 17.20 | C |
| ATOM | 1558 | C | PHE | A | 234 | 36.841 | 6.317 | 11.755 | 1.00 | 16.75 | C |
| ATOM | 1559 | O | PHE | A | 234 | 38.031 | 6.449 | 12.089 | 1.00 | 16.45 | O |
| ATOM | 1560 | N | VAL | A | 235 | 36.102 | 7.298 | 11.258 | 1.00 | 16.26 | N |
| ATOM | 1561 | CA | VAL | A | 235 | 36.720 | 8.581 | 10.942 | 1.00 | 18.10 | C |
| ATOM | 1562 | CB | VAL | A | 235 | 35.788 | 9.506 | 10.082 | 1.00 | 17.90 | C |
| ATOM | 1563 | CG1 | VAL | A | 235 | 36.285 | 10.964 | 10.088 | 1.00 | 19.22 | C |
| ATOM | 1564 | CG2 | VAL | A | 235 | 35.725 | 9.004 | 8.673 | 1.00 | 19.81 | C |
| ATOM | 1565 | C | VAL | A | 235 | 37.288 | 9.286 | 12.189 | 1.00 | 17.93 | C |
| ATOM | 1566 | O | VAL | A | 235 | 38.425 | 9.731 | 12.166 | 1.00 | 19.71 | O |
| ATOM | 1567 | N | ASN | A | 236 | 36.531 | 9.355 | 13.280 | 1.00 | 19.02 | N |
| ATOM | 1568 | CA | ASN | A | 236 | 37.078 | 9.929 | 14.520 | 1.00 | 19.27 | C |
| ATOM | 1569 | CB | ASN | A | 236 | 36.076 | 9.799 | 15.671 | 1.00 | 20.13 | C |
| ATOM | 1570 | CG | ASN | A | 236 | 34.924 | 10.760 | 15.549 | 1.00 | 22.45 | C |
| ATOM | 1571 | OD1 | ASN | A | 236 | 33.828 | 10.492 | 16.039 | 1.00 | 29.23 | O |
| ATOM | 1572 | ND2 | ASN | A | 236 | 35.141 | 11.856 | 14.857 | 1.00 | 25.62 | N |
| ATOM | 1573 | C | ASN | A | 236 | 38.395 | 9.265 | 14.946 | 1.00 | 18.81 | C |
| ATOM | 1574 | O | ASN | A | 236 | 39.327 | 9.960 | 15.354 | 1.00 | 19.24 | O |
| ATOM | 1575 | N | TRP | A | 237 | 38.476 | 7.926 | 14.842 | 1.00 | 17.50 | N |
| ATOM | 1576 | CA | TRP | A | 237 | 39.699 | 7.184 | 15.222 | 1.00 | 16.65 | C |
| ATOM | 1577 | CB | TRP | A | 237 | 39.420 | 5.655 | 15.339 | 1.00 | 16.12 | C |
| ATOM | 1578 | CG | TRP | A | 237 | 40.674 | 4.802 | 15.455 | 1.00 | 16.92 | C |
| ATOM | 1579 | CD1 | TRP | A | 237 | 41.446 | 4.597 | 16.579 | 1.00 | 17.04 | C |
| ATOM | 1580 | NE1 | TRP | A | 237 | 42.516 | 3.788 | 16.274 | 1.00 | 17.49 | N |
| ATOM | 1581 | CE2 | TRP | A | 237 | 42.443 | 3.441 | 14.946 | 1.00 | 16.80 | C |
| ATOM | 1582 | CD2 | TRP | A | 237 | 41.302 | 4.071 | 14.403 | 1.00 | 17.38 | C |
| ATOM | 1583 | CE3 | TRP | A | 237 | 41.000 | 3.863 | 13.040 | 1.00 | 20.43 | C |
| ATOM | 1584 | CZ3 | TRP | A | 237 | 41.850 | 3.055 | 12.283 | 1.00 | 17.48 | C |
| ATOM | 1585 | CH2 | TRP | A | 237 | 42.983 | 2.454 | 12.858 | 1.00 | 18.23 | C |
| ATOM | 1586 | CZ2 | TRP | A | 237 | 43.288 | 2.624 | 14.182 | 1.00 | 17.39 | C |
| ATOM | 1587 | C | TRP | A | 237 | 40.868 | 7.508 | 14.283 | 1.00 | 17.20 | C |
| ATOM | 1588 | O | TRP | A | 237 | 41.985 | 7.803 | 14.752 | 1.00 | 16.35 | O |
| ATOM | 1589 | N | ILE | A | 238 | 40.589 | 7.482 | 12.967 | 1.00 | 17.09 | N |
| ATOM | 1590 | CA | ILE | A | 238 | 41.562 | 7.831 | 11.939 | 1.00 | 17.86 | C |
| ATOM | 1591 | CB | ILE | A | 238 | 40.894 | 7.756 | 10.510 | 1.00 | 18.14 | C |
| ATOM | 1592 | CG1 | ILE | A | 238 | 40.661 | 6.283 | 10.083 | 1.00 | 17.98 | C |
| ATOM | 1593 | CD1 | ILE | A | 238 | 39.728 | 6.119 | 8.830 | 1.00 | 16.57 | C |
| ATOM | 1594 | CG2 | ILE | A | 238 | 41.756 | 8.541 | 9.460 | 1.00 | 16.80 | C |
| ATOM | 1595 | C | ILE | A | 238 | 42.177 | 9.230 | 12.186 | 1.00 | 19.13 | C |
| ATOM | 1596 | O | ILE | A | 238 | 43.406 | 9.372 | 12.217 | 1.00 | 19.67 | O |
| ATOM | 1597 | N | ASP | A | 239 | 41.308 | 10.231 | 12.399 | 1.00 | 20.66 | N |
| ATOM | 1598 | CA | ASP | A | 239 | 41.707 | 11.614 | 12.709 | 1.00 | 21.24 | C |
| ATOM | 1599 | CB | ASP | A | 239 | 40.457 | 12.480 | 12.865 | 1.00 | 21.85 | C |
| ATOM | 1600 | CG | ASP | A | 239 | 39.802 | 12.805 | 11.524 | 1.00 | 22.75 | C |
| ATOM | 1601 | OD1 | ASP | A | 239 | 38.641 | 13.271 | 11.494 | 1.00 | 25.13 | O |
| ATOM | 1602 | OD2 | ASP | A | 239 | 40.379 | 12.588 | 10.448 | 1.00 | 23.36 | O |
| ATOM | 1603 | C | ASP | A | 239 | 42.596 | 11.739 | 13.955 | 1.00 | 22.48 | C |
| ATOM | 1604 | O | ASP | A | 239 | 43.588 | 12.484 | 13.949 | 1.00 | 23.02 | O |
| ATOM | 1605 | N | SER | A | 240 | 42.255 | 10.995 | 15.010 | 1.00 | 22.16 | N |
| ATOM | 1606 | CA | SER | A | 240 | 43.018 | 11.015 | 16.257 | 1.00 | 22.17 | C |
| ATOM | 1607 | CB | SER | A | 240 | 42.386 | 10.068 | 17.292 | 1.00 | 22.40 | C |
| ATOM | 1608 | OG | SER | A | 240 | 42.826 | 8.726 | 17.084 | 1.00 | 21.48 | O |
| ATOM | 1609 | C | SER | A | 240 | 44.467 | 10.624 | 15.967 | 1.00 | 22.89 | C |
| ATOM | 1610 | O | SER | A | 240 | 45.382 | 11.093 | 16.629 | 1.00 | 23.25 | O |
| ATOM | 1611 | N | ILE | A | 241 | 44.675 | 9.793 | 14.954 | 1.00 | 23.49 | N |
| ATOM | 1612 | CA | ILE | A | 241 | 46.013 | 9.300 | 14.628 | 1.00 | 25.19 | C |
| ATOM | 1613 | CB | ILE | A | 241 | 45.937 | 7.832 | 14.139 | 1.00 | 24.99 | C |
| ATOM | 1614 | CG1 | ILE | A | 241 | 45.657 | 6.913 | 15.332 | 1.00 | 24.07 | C |
| ATOM | 1615 | CD1 | ILE | A | 241 | 44.913 | 5.643 | 14.982 | 1.00 | 23.89 | C |
| ATOM | 1616 | CG2 | ILE | A | 241 | 47.237 | 7.390 | 13.454 | 1.00 | 25.03 | C |
| ATOM | 1617 | C | ILE | A | 241 | 46.724 | 10.203 | 13.620 | 1.00 | 27.61 | C |
| ATOM | 1618 | O | ILE | A | 241 | 47.914 | 10.507 | 13.769 | 1.00 | 27.88 | O |
| ATOM | 1619 | N | ILE | A | 242 | 45.961 | 10.659 | 12.630 | 1.00 | 29.81 | N |
| ATOM | 1620 | CA | ILE | A | 242 | 46.464 | 11.363 | 11.446 | 1.00 | 32.08 | C |
| ATOM | 1621 | CB | ILE | A | 242 | 45.531 | 11.000 | 10.255 | 1.00 | 31.70 | C |
| ATOM | 1622 | CG1 | ILE | A | 242 | 46.202 | 9.955 | 9.402 | 1.00 | 30.80 | C |
| ATOM | 1623 | CD1 | ILE | A | 242 | 46.407 | 8.734 | 10.134 | 1.00 | 28.38 | C |
| ATOM | 1624 | CG2 | ILE | A | 242 | 45.113 | 12.152 | 9.413 | 1.00 | 33.80 | C |
| ATOM | 1625 | C | ILE | A | 242 | 46.592 | 12.872 | 11.656 | 1.00 | 34.00 | C |
| ATOM | 1626 | O | ILE | A | 242 | 47.575 | 13.463 | 11.203 | 1.00 | 34.58 | O |
| ATOM | 1627 | N | GLN | A | 243 | 45.598 | 13.468 | 12.326 | 1.00 | 35.57 | N |
| ATOM | 1628 | CA | GLN | A | 243 | 45.517 | 14.913 | 12.545 | 1.00 | 37.73 | C |
| ATOM | 1629 | CB | GLN | A | 243 | 44.081 | 15.431 | 12.364 | 1.00 | 38.02 | C |
| ATOM | 1630 | CG | GLN | A | 243 | 43.185 | 14.548 | 11.464 | 1.00 | 40.80 | C |
| ATOM | 1631 | CD | GLN | A | 243 | 42.675 | 15.215 | 10.174 | 1.00 | 45.03 | C |
| ATOM | 1632 | OE1 | GLN | A | 243 | 41.683 | 14.753 | 9.588 | 1.00 | 46.82 | O |
| ATOM | 1633 | NE2 | GLN | A | 243 | 43.350 | 16.279 | 9.724 | 1.00 | 46.94 | N |
| ATOM | 1634 | C | GLN | A | 243 | 46.034 | 15.259 | 13.936 | 1.00 | 38.24 | C |
| ATOM | 1635 | O | GLN | A | 243 | 47.205 | 14.988 | 14.249 | 1.00 | 39.40 | O |
| ATOM | 1636 | C1 | NAG | A | 401 | 31.053 | 4.461 | −13.545 | 1.00 | 19.91 | C |
| ATOM | 1637 | C2 | NAG | A | 401 | 29.716 | 5.171 | −13.802 | 1.00 | 23.14 | C |
| ATOM | 1638 | N2 | NAG | A | 401 | 28.836 | 4.489 | −14.726 | 1.00 | 23.84 | N |
| ATOM | 1639 | C7 | NAG | A | 401 | 27.767 | 3.807 | −14.321 | 1.00 | 27.43 | C |
| ATOM | 1640 | O7 | NAG | A | 401 | 27.486 | 3.614 | −13.150 | 1.00 | 28.14 | O |
| ATOM | 1641 | C8 | NAG | A | 401 | 26.872 | 3.219 | −15.371 | 1.00 | 29.09 | C |
| ATOM | 1642 | C3 | NAG | A | 401 | 30.042 | 6.568 | −14.357 | 1.00 | 25.61 | C |
| ATOM | 1643 | O3 | NAG | A | 401 | 28.856 | 7.321 | −14.494 | 1.00 | 25.83 | O |
| ATOM | 1644 | C4 | NAG | A | 401 | 31.028 | 7.278 | −13.425 | 1.00 | 25.11 | C |
| ATOM | 1645 | O4 | NAG | A | 401 | 31.536 | 8.427 | −14.070 | 1.00 | 29.40 | O |
| ATOM | 1646 | C5 | NAG | A | 401 | 32.233 | 6.409 | −13.046 | 1.00 | 23.07 | C |
| ATOM | 1647 | C6 | NAG | A | 401 | 33.070 | 7.097 | −11.980 | 1.00 | 21.74 | C |
| ATOM | 1648 | O6 | NAG | A | 401 | 32.224 | 7.229 | −10.843 | 1.00 | 23.50 | O |
| ATOM | 1649 | O5 | NAG | A | 401 | 31.715 | 5.212 | −12.542 | 1.00 | 22.24 | O |
| ATOM | 1650 | C1 | FUC | A | 402 | 32.240 | 8.497 | −10.162 | 1.00 | 22.08 | C |
| ATOM | 1651 | C2 | FUC | A | 402 | 31.279 | 8.379 | −8.958 | 1.00 | 22.79 | C |
| ATOM | 1652 | O2 | FUC | A | 402 | 29.998 | 7.851 | −9.282 | 1.00 | 22.82 | O |
| ATOM | 1653 | C3 | FUC | A | 402 | 31.858 | 7.497 | −7.847 | 1.00 | 19.62 | C |
| ATOM | 1654 | O3 | FUC | A | 402 | 30.999 | 7.615 | −6.743 | 1.00 | 21.40 | O |
| ATOM | 1655 | C4 | FUC | A | 402 | 33.269 | 7.943 | −7.491 | 1.00 | 18.08 | C |
| ATOM | 1656 | O4 | FUC | A | 402 | 33.177 | 9.219 | −6.905 | 1.00 | 15.77 | O |
| ATOM | 1657 | C5 | FUC | A | 402 | 34.139 | 7.998 | −8.753 | 1.00 | 20.39 | C |
| ATOM | 1658 | C6 | FUC | A | 402 | 35.531 | 8.538 | −8.438 | 1.00 | 22.13 | C |
| ATOM | 1659 | O5 | FUC | A | 402 | 33.539 | 8.864 | −9.709 | 1.00 | 22.73 | O |
| ATOM | 1660 | C1 | NAG | A | 403 | 30.540 | 10.080 | −13.548 | 1.00 | 49.45 | C |
| ATOM | 1661 | C2 | NAG | A | 403 | 31.705 | 10.987 | −13.904 | 1.00 | 48.83 | C |
| ATOM | 1662 | N2 | NAG | A | 403 | 32.580 | 10.989 | −12.766 | 1.00 | 43.86 | N |
| ATOM | 1663 | C7 | NAG | A | 403 | 33.890 | 10.816 | −12.846 | 1.00 | 44.28 | C |
| ATOM | 1664 | O7 | NAG | A | 403 | 34.474 | 10.361 | −13.837 | 1.00 | 43.92 | O |
| ATOM | 1665 | C8 | NAG | A | 403 | 34.656 | 11.223 | −11.618 | 1.00 | 43.21 | C |
| ATOM | 1666 | C3 | NAG | A | 403 | 31.142 | 12.374 | −14.226 | 1.00 | 50.99 | C |
| ATOM | 1667 | O3 | NAG | A | 403 | 32.189 | 13.193 | −14.705 | 1.00 | 50.55 | O |
| ATOM | 1668 | C4 | NAG | A | 403 | 30.017 | 12.268 | −15.270 | 1.00 | 53.18 | C |
| ATOM | 1669 | O4 | NAG | A | 403 | 29.392 | 13.530 | −15.439 | 1.00 | 55.41 | O |
| ATOM | 1670 | C5 | NAG | A | 403 | 28.989 | 11.185 | −14.887 | 1.00 | 54.16 | C |
| ATOM | 1671 | C6 | NAG | A | 403 | 27.859 | 11.031 | −15.918 | 1.00 | 54.94 | C |
| ATOM | 1672 | O6 | NAG | A | 403 | 27.897 | 9.794 | −16.603 | 1.00 | 56.56 | O |
| ATOM | 1673 | O5 | NAG | A | 403 | 29.668 | 9.958 | −14.644 | 1.00 | 52.52 | O |
| ATOM | 1674 | C1 | NAG | A | 411 | 62.027 | 4.314 | 4.206 | 1.00 | 42.36 | C |
| ATOM | 1675 | C2 | NAG | A | 411 | 63.383 | 5.047 | 4.092 | 1.00 | 48.20 | C |
| ATOM | 1676 | N2 | NAG | A | 411 | 63.217 | 6.478 | 4.330 | 1.00 | 48.97 | N |
| ATOM | 1677 | C7 | NAG | A | 411 | 63.721 | 7.130 | 5.379 | 1.00 | 50.32 | C |
| ATOM | 1678 | O7 | NAG | A | 411 | 63.624 | 8.352 | 5.467 | 1.00 | 52.45 | O |
| ATOM | 1679 | C8 | NAG | A | 411 | 64.412 | 6.351 | 6.466 | 1.00 | 50.07 | C |
| ATOM | 1680 | C3 | NAG | A | 411 | 64.118 | 4.839 | 2.753 | 1.00 | 48.47 | C |
| ATOM | 1681 | O3 | NAG | A | 411 | 65.497 | 5.121 | 2.919 | 1.00 | 50.28 | O |
| ATOM | 1682 | C4 | NAG | A | 411 | 63.991 | 3.420 | 2.185 | 1.00 | 47.94 | C |
| ATOM | 1683 | O4 | NAG | A | 411 | 64.317 | 3.472 | 0.810 | 1.00 | 50.18 | O |
| ATOM | 1684 | C5 | NAG | A | 411 | 62.592 | 2.838 | 2.410 | 1.00 | 45.83 | C |
| ATOM | 1685 | C6 | NAG | A | 411 | 62.444 | 1.395 | 1.894 | 1.00 | 44.68 | C |
| ATOM | 1686 | O6 | NAG | A | 411 | 63.193 | 0.440 | 2.618 | 1.00 | 44.35 | O |
| ATOM | 1687 | O5 | NAG | A | 411 | 62.230 | 2.975 | 3.788 | 1.00 | 43.22 | O |
| ATOM | 1688 | F1 | RSU | B | 500 | 39.134 | −7.820 | −1.728 | 1.00 | 13.34 | F |
| ATOM | 1689 | C11 | RSU | B | 500 | 38.628 | −8.423 | −0.657 | 1.00 | 14.67 | C |
| ATOM | 1690 | F3 | RSU | B | 500 | 39.137 | −7.810 | 0.403 | 1.00 | 15.27 | F |
| ATOM | 1691 | F2 | RSU | B | 500 | 37.317 | −8.239 | −0.574 | 1.00 | 11.56 | F |
| ATOM | 1692 | C7 | RSU | B | 500 | 38.825 | −9.941 | −0.690 | 1.00 | 11.43 | C |
| ATOM | 1693 | C8 | RSU | B | 500 | 38.758 | −10.637 | −1.895 | 1.00 | 12.63 | C |
| ATOM | 1694 | C9 | RSU | B | 500 | 38.887 | −12.050 | −1.895 | 1.00 | 12.18 | C |
| ATOM | 1695 | C10 | RSU | B | 500 | 39.091 | −12.761 | −0.717 | 1.00 | 11.90 | C |
| ATOM | 1696 | C6 | RSU | B | 500 | 39.010 | −10.673 | 0.492 | 1.00 | 11.86 | C |
| ATOM | 1697 | C5 | RSU | B | 500 | 39.172 | −12.078 | 0.496 | 1.00 | 13.18 | C |
| ATOM | 1698 | N1 | RSU | B | 500 | 39.346 | −12.692 | 1.757 | 1.00 | 13.97 | N |
| ATOM | 1699 | C4 | RSU | B | 500 | 40.573 | −12.758 | 2.362 | 1.00 | 16.63 | C |
| ATOM | 1700 | C23 | RSU | B | 500 | 41.779 | −12.184 | 1.639 | 1.00 | 13.37 | C |
| ATOM | 1701 | C3 | RSU | B | 500 | 40.694 | −13.344 | 3.583 | 1.00 | 19.62 | C |
| ATOM | 1702 | C20 | RSU | B | 500 | 41.909 | −13.496 | 4.403 | 1.00 | 21.70 | C |
| ATOM | 1703 | O3 | RSU | B | 500 | 41.684 | −14.097 | 5.717 | 1.00 | 30.85 | O |
| ATOM | 1704 | C21 | RSU | B | 500 | 42.730 | −14.597 | 6.566 | 1.00 | 34.80 | C |
| ATOM | 1705 | C22 | RSU | B | 500 | 43.301 | −13.400 | 7.299 | 1.00 | 39.99 | C |
| ATOM | 1706 | O33 | RSU | B | 500 | 44.670 | −13.578 | 7.675 | 1.00 | 46.22 | O |
| ATOM | 1707 | O2 | RSU | B | 500 | 43.033 | −13.189 | 4.023 | 1.00 | 21.85 | O |
| ATOM | 1708 | C1 | RSU | B | 500 | 38.235 | −13.228 | 2.315 | 1.00 | 14.64 | C |
| ATOM | 1709 | O1 | RSU | B | 500 | 37.146 | −13.189 | 1.763 | 1.00 | 13.12 | O |
| ATOM | 1710 | N2 | RSU | B | 500 | 38.302 | −13.827 | 3.499 | 1.00 | 18.78 | N |
| ATOM | 1711 | C2 | RSU | B | 500 | 39.507 | −13.830 | 4.304 | 1.00 | 18.31 | C |
| ATOM | 1712 | C13 | RSU | B | 500 | 39.154 | −12.746 | 5.327 | 1.00 | 21.25 | C |
| ATOM | 1713 | C14 | RSU | B | 500 | 38.877 | −11.399 | 4.965 | 1.00 | 20.38 | C |
| ATOM | 1714 | C15 | RSU | B | 500 | 38.505 | −10.433 | 5.920 | 1.00 | 20.30 | C |
| ATOM | 1715 | C16 | RSU | B | 500 | 38.424 | −10.795 | 7.277 | 1.00 | 23.27 | C |
| ATOM | 1716 | C19 | RSU | B | 500 | 38.077 | −9.934 | 8.161 | 1.00 | 23.09 | C |
| ATOM | 1717 | N3 | RSU | B | 500 | 37.758 | −9.115 | 8.920 | 1.00 | 20.63 | N |
| ATOM | 1718 | C17 | RSU | B | 500 | 38.689 | −12.112 | 7.666 | 1.00 | 24.08 | C |
| ATOM | 1719 | C18 | RSU | B | 500 | 39.045 | −13.055 | 6.693 | 1.00 | 22.87 | C |
| ATOM | 1720 | O | HOH | W | 1 | 49.368 | 7.312 | −9.061 | 1.00 | 12.52 | O |
| ATOM | 1721 | O | HOH | W | 2 | 38.399 | −1.645 | −4.131 | 1.00 | 11.49 | O |
| ATOM | 1722 | O | HOH | W | 3 | 50.775 | 3.170 | −5.865 | 1.00 | 12.83 | O |
| ATOM | 1723 | O | HOH | W | 4 | 45.973 | 5.632 | −9.937 | 1.00 | 11.06 | O |
| ATOM | 1724 | O | HOH | W | 5 | 42.846 | −6.122 | −10.442 | 1.00 | 10.81 | O |
| ATOM | 1725 | O | HOH | W | 6 | 31.320 | −11.224 | −2.760 | 1.00 | 8.46 | O |
| ATOM | 1726 | O | HOH | W | 7 | 33.852 | −10.375 | −5.953 | 1.00 | 11.13 | O |
| ATOM | 1727 | O | HOH | W | 8 | 31.233 | −5.308 | −5.989 | 1.00 | 10.77 | O |
| ATOM | 1728 | O | HOH | W | 9 | 47.659 | 4.316 | −11.539 | 1.00 | 21.92 | O |
| ATOM | 1729 | O | HOH | W | 10 | 29.767 | −7.321 | −7.289 | 1.00 | 10.19 | O |
| ATOM | 1730 | O | HOH | W | 11 | 44.636 | 12.281 | 19.031 | 1.00 | 14.38 | O |
| ATOM | 1731 | O | HOH | W | 12 | 35.821 | 6.389 | 14.950 | 1.00 | 16.32 | O |
| ATOM | 1732 | O | HOH | W | 13 | 43.687 | −10.524 | −0.752 | 1.00 | 14.73 | O |
| ATOM | 1733 | O | HOH | W | 14 | 45.096 | −3.870 | −4.769 | 1.00 | 9.68 | O |
| ATOM | 1734 | O | HOH | W | 15 | 40.370 | 5.749 | −14.803 | 1.00 | 15.39 | O |
| ATOM | 1735 | O | HOH | W | 16 | 32.660 | −1.232 | −14.697 | 1.00 | 15.66 | O |
| ATOM | 1736 | O | HOH | W | 17 | 31.069 | −12.866 | −4.896 | 1.00 | 15.02 | O |
| ATOM | 1737 | O | HOH | W | 18 | 30.961 | −10.529 | 0.853 | 1.00 | 15.06 | O |
| ATOM | 1738 | O | HOH | W | 19 | 48.489 | 5.036 | −8.017 | 1.00 | 10.88 | O |
| ATOM | 1739 | O | HOH | W | 20 | 29.519 | −10.794 | −7.950 | 1.00 | 12.80 | O |
| ATOM | 1740 | O | HOH | W | 21 | 47.204 | 15.412 | −1.575 | 1.00 | 15.23 | O |
| ATOM | 1741 | O | HOH | W | 22 | 48.160 | −10.992 | 3.756 | 1.00 | 27.84 | O |
| ATOM | 1742 | O | HOH | W | 23 | 39.389 | −6.305 | −14.157 | 1.00 | 12.86 | O |
| ATOM | 1743 | O | HOH | W | 24 | 19.724 | −3.726 | −1.306 | 1.00 | 25.28 | O |
| ATOM | 1744 | O | HOH | W | 25 | 42.387 | 1.477 | 1.939 | 1.00 | 14.42 | O |
| ATOM | 1745 | O | HOH | W | 26 | 16.634 | −7.128 | 2.246 | 1.00 | 27.57 | O |
| ATOM | 1746 | O | HOH | W | 27 | 33.678 | −0.912 | 15.627 | 1.00 | 17.17 | O |
| ATOM | 1747 | O | HOH | W | 28 | 34.619 | −6.542 | 7.358 | 1.00 | 16.36 | O |
| ATOM | 1748 | O | HOH | W | 29 | 31.820 | 4.549 | 4.873 | 1.00 | 19.25 | O |
| ATOM | 1749 | O | HOH | W | 30 | 35.253 | −4.024 | 8.861 | 1.00 | 14.53 | O |
| ATOM | 1750 | O | HOH | W | 31 | 29.943 | −9.504 | −5.615 | 1.00 | 13.15 | O |
| ATOM | 1751 | O | HOH | W | 32 | 28.770 | −17.167 | −10.524 | 1.00 | 22.27 | O |
| ATOM | 1752 | O | HOH | W | 33 | 39.604 | 2.876 | 19.251 | 1.00 | 12.71 | O |
| ATOM | 1753 | O | HOH | W | 34 | 34.281 | −16.852 | −1.094 | 1.00 | 24.60 | O |
| ATOM | 1754 | O | HOH | W | 35 | 41.554 | −3.044 | 13.516 | 1.00 | 19.46 | O |
| ATOM | 1755 | O | HOH | W | 36 | 25.952 | −1.586 | 2.433 | 1.00 | 15.01 | O |
| ATOM | 1756 | O | HOH | W | 37 | 33.099 | −3.496 | 10.603 | 1.00 | 15.87 | O |
| ATOM | 1757 | O | HOH | W | 38 | 37.079 | −7.021 | −17.649 | 1.00 | 25.07 | O |
| ATOM | 1758 | O | HOH | W | 39 | 32.556 | −17.251 | −7.558 | 1.00 | 22.84 | O |
| ATOM | 1759 | O | HOH | W | 40 | 28.634 | 3.123 | 8.022 | 1.00 | 27.59 | O |
| ATOM | 1760 | O | HOH | W | 41 | 25.220 | −2.317 | 4.953 | 1.00 | 22.45 | O |
| ATOM | 1761 | O | HOH | W | 42 | 30.400 | −2.642 | −13.557 | 1.00 | 18.11 | O |
| ATOM | 1762 | O | HOH | W | 43 | 27.955 | −9.395 | −9.940 | 1.00 | 18.54 | O |
| ATOM | 1763 | O | HOH | W | 44 | 49.889 | 6.602 | −4.615 | 1.00 | 16.01 | O |
| ATOM | 1764 | O | HOH | W | 45 | 30.545 | 5.255 | −9.997 | 1.00 | 27.17 | O |
| ATOM | 1765 | O | HOH | W | 46 | 58.017 | −2.068 | 6.419 | 1.00 | 20.46 | O |
| ATOM | 1766 | O | HOH | W | 47 | 56.252 | 10.567 | −7.604 | 1.00 | 22.80 | O |
| ATOM | 1767 | O | HOH | W | 48 | 41.353 | 10.921 | −8.330 | 1.00 | 24.88 | O |
| ATOM | 1768 | O | HOH | W | 49 | 43.457 | −12.633 | 10.099 | 1.00 | 29.57 | O |
| ATOM | 1769 | O | HOH | W | 50 | 57.234 | 15.764 | −7.332 | 1.00 | 28.67 | O |
| ATOM | 1770 | O | HOH | W | 51 | 25.341 | 9.364 | 1.846 | 1.00 | 31.16 | O |
| ATOM | 1771 | O | HOH | W | 52 | 48.330 | −12.243 | −14.397 | 1.00 | 26.56 | O |
| ATOM | 1772 | O | HOH | W | 53 | 29.087 | 2.826 | −10.207 | 1.00 | 29.39 | O |
| ATOM | 1773 | O | HOH | W | 54 | 19.934 | −8.344 | 4.158 | 1.00 | 23.64 | O |
| ATOM | 1774 | O | HOH | W | 55 | 54.853 | 16.365 | 0.169 | 1.00 | 20.43 | O |
| ATOM | 1775 | O | HOH | W | 56 | 37.308 | −3.882 | −19.408 | 1.00 | 21.37 | O |
| ATOM | 1776 | O | HOH | W | 57 | 45.710 | 14.229 | 5.893 | 1.00 | 28.12 | O |
| ATOM | 1777 | O | HOH | W | 58 | 30.656 | 12.530 | 4.042 | 1.00 | 24.94 | O |
| ATOM | 1778 | O | HOH | W | 59 | 36.918 | −17.278 | −6.541 | 1.00 | 25.03 | O |
| ATOM | 1779 | O | HOH | W | 60 | 25.818 | 1.223 | 3.086 | 1.00 | 23.83 | O |
| ATOM | 1780 | O | HOH | W | 61 | 41.163 | −5.403 | −18.147 | 1.00 | 28.33 | O |
| ATOM | 1781 | O | HOH | W | 62 | 46.668 | −6.123 | −16.118 | 1.00 | 29.89 | O |
| ATOM | 1782 | O | HOH | W | 63 | 34.176 | −8.195 | 17.745 | 1.00 | 25.41 | O |
| ATOM | 1783 | O | HOH | W | 64 | 27.246 | 3.085 | 4.754 | 1.00 | 25.34 | O |
| ATOM | 1784 | O | HOH | W | 65 | 54.099 | −6.802 | −7.621 | 1.00 | 31.75 | O |
| ATOM | 1785 | O | HOH | W | 66 | 31.825 | −18.162 | 0.711 | 1.00 | 20.34 | O |
| ATOM | 1786 | O | HOH | W | 67 | 43.154 | −0.288 | 19.396 | 1.00 | 24.12 | O |
| ATOM | 1787 | O | HOH | W | 68 | 28.459 | −7.802 | 5.821 | 1.00 | 33.82 | O |
| ATOM | 1788 | O | HOH | W | 69 | 29.457 | 4.211 | 5.730 | 1.00 | 23.31 | O |
| ATOM | 1789 | O | HOH | W | 70 | 39.352 | 10.876 | −2.300 | 1.00 | 27.69 | O |
| ATOM | 1790 | O | HOH | W | 71 | 33.985 | 13.753 | 10.878 | 1.00 | 28.40 | O |
| ATOM | 1791 | O | HOH | W | 72 | 59.606 | 10.076 | −1.497 | 1.00 | 38.06 | O |
| ATOM | 1792 | O | HOH | W | 73 | 28.014 | −3.345 | −15.290 | 1.00 | 31.87 | O |
| ATOM | 1793 | O | HOH | W | 74 | 39.576 | 14.061 | 4.227 | 1.00 | 32.41 | O |
| ATOM | 1794 | O | HOH | W | 75 | 28.414 | 4.263 | 10.592 | 1.00 | 44.60 | O |
| ATOM | 1795 | O | HOH | W | 76 | 52.788 | −10.881 | −5.648 | 1.00 | 25.00 | O |
| ATOM | 1796 | O | HOH | W | 77 | 38.929 | 12.289 | 16.408 | 1.00 | 27.89 | O |
| ATOM | 1797 | O | HOH | W | 78 | 54.207 | 18.434 | −6.247 | 1.00 | 27.30 | O |
| ATOM | 1798 | O | HOH | W | 79 | 44.605 | 2.549 | 17.562 | 1.00 | 28.87 | O |
| ATOM | 1799 | O | HOH | W | 80 | 42.734 | 11.486 | 4.578 | 1.00 | 18.69 | O |
| ATOM | 1800 | O | HOH | W | 81 | 27.181 | −1.130 | −10.835 | 1.00 | 27.99 | O |
| ATOM | 1801 | O | HOH | W | 82 | 35.744 | −11.741 | −16.471 | 1.00 | 28.42 | O |
| ATOM | 1802 | O | HOH | W | 83 | 19.647 | −11.770 | −2.561 | 1.00 | 34.96 | O |
| ATOM | 1803 | O | HOH | W | 84 | 55.443 | 11.609 | −9.932 | 1.00 | 33.52 | O |
| ATOM | 1804 | O | HOH | W | 85 | 37.815 | −6.927 | 18.938 | 1.00 | 28.08 | O |
| ATOM | 1805 | O | HOH | W | 86 | 50.289 | 13.512 | 9.058 | 1.00 | 28.75 | O |
| ATOM | 1806 | O | HOH | W | 87 | 53.626 | 5.417 | −14.801 | 1.00 | 37.38 | O |
| ATOM | 1807 | O | HOH | W | 88 | 40.995 | 12.952 | 5.975 | 1.00 | 35.62 | O |
| ATOM | 1808 | O | HOH | W | 89 | 56.279 | 9.127 | 9.654 | 1.00 | 24.74 | O |
| ATOM | 1809 | O | HOH | W | 90 | 52.710 | 12.394 | 12.091 | 1.00 | 37.01 | O |
| ATOM | 1810 | O | HOH | W | 91 | 36.521 | 6.157 | −14.084 | 1.00 | 32.88 | O |
| ATOM | 1811 | O | HOH | W | 92 | 56.205 | 5.959 | −14.426 | 1.00 | 32.89 | O |
| ATOM | 1812 | O | HOH | W | 93 | 38.360 | −9.792 | −15.344 | 1.00 | 33.06 | O |
| ATOM | 1813 | O | HOH | W | 94 | 60.425 | −1.673 | −17.064 | 1.00 | 43.17 | O |
| ATOM | 1814 | O | HOH | W | 95 | 44.975 | 2.344 | −19.004 | 1.00 | 29.51 | O |
| ATOM | 1815 | O | HOH | W | 96 | 40.084 | −2.739 | −19.784 | 1.00 | 34.49 | O |
| ATOM | 1816 | O | HOH | W | 97 | 23.034 | −18.238 | 0.289 | 1.00 | 20.65 | O |
| ATOM | 1817 | O | HOH | W | 98 | 38.115 | −18.805 | −4.690 | 1.00 | 47.69 | O |
| ATOM | 1818 | O | HOH | W | 99 | 26.216 | 5.823 | 11.414 | 1.00 | 35.98 | O |
| ATOM | 1819 | O | HOH | W | 100 | 39.490 | 3.623 | −17.186 | 1.00 | 31.68 | O |
| ATOM | 1820 | O | HOH | W | 101 | 32.928 | 11.578 | −7.961 | 1.00 | 29.84 | O |
| ATOM | 1821 | O | HOH | W | 102 | 59.284 | 12.582 | 0.227 | 1.00 | 37.63 | O |
| ATOM | 1822 | O | HOH | W | 103 | 19.405 | −5.109 | −3.305 | 1.00 | 41.89 | O |
| ATOM | 1823 | O | HOH | W | 104 | 58.820 | 0.123 | −7.002 | 1.00 | 41.21 | O |
| ATOM | 1824 | O | HOH | W | 105 | 31.317 | 13.296 | −6.270 | 1.00 | 29.12 | O |
| ATOM | 1825 | O | HOH | W | 106 | 23.329 | 0.129 | 14.524 | 1.00 | 44.71 | O |
| ATOM | 1826 | O | HOH | W | 107 | 36.702 | 12.973 | 7.302 | 1.00 | 37.59 | O |
| ATOM | 1827 | O | HOH | W | 108 | 50.889 | 1.907 | 17.859 | 1.00 | 43.58 | O |
| ATOM | 1828 | O | HOH | W | 109 | 42.380 | 13.257 | −3.089 | 1.00 | 30.53 | O |
| ATOM | 1829 | O | HOH | W | 110 | 21.171 | −1.216 | −4.871 | 1.00 | 44.14 | O |
| ATOM | 1830 | O | HOH | W | 111 | 46.307 | −9.114 | 13.565 | 1.00 | 39.14 | O |
| ATOM | 1831 | O | HOH | W | 112 | 25.445 | −2.844 | −11.527 | 1.00 | 37.83 | O |
| ATOM | 1832 | O | HOH | W | 113 | 27.256 | 0.557 | 6.204 | 1.00 | 45.71 | O |
| ATOM | 1833 | O | HOH | W | 114 | 22.835 | −3.654 | 5.604 | 1.00 | 31.76 | O |
| ATOM | 1834 | O | HOH | W | 115 | 42.029 | −7.172 | 17.442 | 1.00 | 35.85 | O |
| ATOM | 1835 | O | HOH | W | 116 | 19.146 | 3.057 | 1.530 | 1.00 | 32.76 | O |
| ATOM | 1836 | O | HOH | W | 117 | 43.619 | −5.192 | 17.196 | 1.00 | 31.92 | O |
| ATOM | 1837 | O | HOH | W | 118 | 42.410 | −23.188 | −10.245 | 1.00 | 40.96 | O |
| ATOM | 1838 | O | HOH | W | 119 | 36.524 | −15.835 | 0.649 | 1.00 | 32.82 | O |
| ATOM | 1839 | O | HOH | W | 120 | 24.451 | −9.799 | −9.824 | 1.00 | 34.61 | O |
| ATOM | 1840 | O | HOH | W | 121 | 31.552 | −14.289 | 11.083 | 1.00 | 54.69 | O |
| ATOM | 1841 | O | HOH | W | 122 | 55.987 | −3.650 | −8.549 | 1.00 | 33.05 | O |
| ATOM | 1842 | O | HOH | W | 123 | 32.303 | 1.030 | 14.326 | 1.00 | 29.27 | O |
| ATOM | 1843 | O | HOH | W | 124 | 25.451 | 1.930 | 10.847 | 1.00 | 45.42 | O |
| ATOM | 1844 | O | HOH | W | 125 | 33.060 | −12.252 | −15.089 | 1.00 | 33.65 | O |
| ATOM | 1845 | O | HOH | W | 126 | 32.640 | −13.894 | 18.700 | 1.00 | 32.70 | O |
| ATOM | 1846 | O | HOH | W | 127 | 33.344 | 14.451 | −5.572 | 1.00 | 39.91 | O |
| ATOM | 1847 | O | HOH | W | 128 | 52.238 | −7.594 | −17.405 | 1.00 | 38.53 | O |
| ATOM | 1848 | O | HOH | W | 129 | 45.882 | −20.216 | −16.229 | 1.00 | 31.77 | O |
| ATOM | 1849 | O | HOH | W | 130 | 28.380 | −0.063 | −14.944 | 1.00 | 34.32 | O |
| ATOM | 1850 | O | HOH | W | 131 | 53.838 | 3.052 | −16.114 | 1.00 | 38.10 | O |
| ATOM | 1851 | O | HOH | W | 132 | 43.109 | −2.904 | 18.461 | 1.00 | 37.20 | O |
| ATOM | 1852 | O | HOH | W | 133 | 44.366 | −5.390 | −17.946 | 1.00 | 40.21 | O |
| ATOM | 1853 | O | HOH | W | 134 | 37.139 | 14.206 | 9.637 | 1.00 | 31.57 | O |
| ATOM | 1854 | O | HOH | W | 135 | 39.366 | 13.376 | 8.040 | 1.00 | 44.10 | O |
| ATOM | 1855 | O | HOH | W | 136 | 33.303 | 11.815 | −17.303 | 1.00 | 51.27 | O |
| ATOM | 1856 | O | HOH | W | 137 | 57.574 | 2.952 | −16.275 | 1.00 | 44.77 | O |
| ATOM | 1857 | O | HOH | W | 138 | 27.697 | 8.869 | −7.608 | 1.00 | 36.37 | O |
| ATOM | 1858 | O | HOH | W | 139 | 34.425 | −19.240 | −8.267 | 1.00 | 38.24 | O |
| ATOM | 1859 | O | HOH | W | 140 | 47.006 | −0.051 | 17.928 | 1.00 | 33.49 | O |
| ATOM | 1860 | O | HOH | W | 141 | 46.064 | −7.271 | 11.525 | 1.00 | 27.35 | O |
| ATOM | 1861 | O | HOH | W | 142 | 61.289 | 6.035 | −3.040 | 1.00 | 34.38 | O |
| ATOM | 1862 | O | HOH | W | 143 | 53.373 | 6.650 | 21.718 | 1.00 | 47.99 | O |
| ATOM | 1863 | O | HOH | W | 144 | 21.391 | −2.660 | 13.655 | 1.00 | 37.89 | O |
| ATOM | 1864 | O | HOH | W | 145 | 47.353 | 10.281 | −16.489 | 1.00 | 41.65 | O |
| ATOM | 1865 | O | HOH | W | 146 | 48.418 | 16.052 | 12.421 | 1.00 | 59.73 | O |
| ATOM | 1866 | O | HOH | W | 147 | 63.288 | 4.156 | 7.885 | 1.00 | 42.98 | O |
| ATOM | 1867 | O | HOH | W | 148 | 55.779 | −12.128 | −1.939 | 1.00 | 40.23 | O |
| ATOM | 1868 | O | HOH | W | 149 | 49.369 | 12.271 | −14.074 | 1.00 | 39.77 | O |
| ATOM | 1869 | O | HOH | W | 150 | 21.957 | −11.052 | −4.933 | 1.00 | 33.10 | O |
| ATOM | 1870 | O | HOH | W | 151 | 60.400 | −5.462 | 14.842 | 1.00 | 50.24 | O |
| ATOM | 1871 | O | HOH | W | 152 | 40.653 | 10.714 | −11.920 | 1.00 | 35.03 | O |
| ATOM | 1872 | O | HOH | W | 153 | 49.521 | 9.480 | 15.670 | 1.00 | 43.52 | O |
| ATOM | 1873 | O | HOH | W | 154 | 40.960 | −13.681 | −20.900 | 1.00 | 48.95 | O |
| ATOM | 1874 | O | HOH | W | 155 | 42.318 | −17.986 | −9.639 | 1.00 | 43.09 | O |
| ATOM | 1875 | O | HOH | W | 156 | 18.800 | −5.429 | −11.668 | 1.00 | 62.09 | O |
| ATOM | 1876 | O | HOH | W | 157 | 43.312 | −14.039 | −20.695 | 1.00 | 41.55 | O |
| ATOM | 1877 | O | HOH | W | 158 | 31.920 | 12.136 | −10.254 | 1.00 | 31.85 | O |
| ATOM | 1878 | O | HOH | W | 159 | 48.889 | 18.075 | 14.344 | 1.00 | 48.62 | O |
| ATOM | 1879 | O | HOH | W | 160 | 32.046 | −20.258 | −6.472 | 1.00 | 43.57 | O |
| ATOM | 1880 | O | HOH | W | 161 | 27.184 | 0.834 | −12.824 | 1.00 | 34.15 | O |
| ATOM | 1881 | O | HOH | W | 162 | 52.803 | 18.029 | −9.636 | 1.00 | 39.25 | O |
| ATOM | 1882 | O | HOH | W | 163 | 36.507 | 16.225 | 1.289 | 1.00 | 46.88 | O |
| ATOM | 1883 | O | HOH | W | 164 | 22.930 | 0.629 | −10.180 | 1.00 | 42.07 | O |
| ATOM | 1884 | O | HOH | W | 165 | 39.852 | −16.455 | −8.727 | 1.00 | 34.95 | O |
| ATOM | 1885 | O | HOH | W | 166 | 23.772 | −14.002 | −10.509 | 1.00 | 29.03 | O |
| ATOM | 1886 | O | HOH | W | 167 | 21.179 | −6.938 | −2.952 | 1.00 | 33.82 | O |
| ATOM | 1887 | O | HOH | W | 168 | 35.923 | −15.076 | 4.833 | 1.00 | 32.81 | O |
| ATOM | 1888 | O | HOH | W | 169 | 41.381 | −21.425 | −12.348 | 1.00 | 40.31 | O |
| ATOM | 1889 | O | HOH | W | 170 | 28.648 | 9.762 | −11.309 | 1.00 | 40.41 | O |
| ATOM | 1890 | O | HOH | W | 171 | 27.691 | 15.306 | −14.607 | 1.00 | 44.06 | O |
| ATOM | 1891 | O | HOH | W | 172 | 20.427 | −7.504 | −7.390 | 1.00 | 48.30 | O |
| ATOM | 1892 | O | HOH | W | 173 | 43.615 | −23.957 | −14.733 | 1.00 | 40.78 | O |
| ATOM | 1893 | O | HOH | W | 174 | 34.467 | 14.808 | 4.090 | 1.00 | 35.50 | O |
| ATOM | 1894 | O | HOH | W | 175 | 37.905 | −15.211 | 9.706 | 1.00 | 41.93 | O |
| ATOM | 1895 | O | HOH | W | 176 | 60.538 | 12.120 | 4.554 | 1.00 | 34.06 | O |
| ATOM | 1896 | O | HOH | W | 177 | 34.405 | 8.105 | 18.788 | 1.00 | 33.40 | O |
| ATOM | 1897 | O | HOH | W | 178 | 47.739 | −4.504 | 16.391 | 1.00 | 57.12 | O |
| ATOM | 1898 | O | HOH | W | 179 | 41.992 | −24.008 | −13.041 | 1.00 | 49.31 | O |
| ATOM | 1899 | O | HOH | W | 180 | 42.317 | 14.927 | 2.062 | 1.00 | 41.31 | O |
| ATOM | 1900 | O | HOH | W | 181 | 27.580 | 14.003 | 0.828 | 1.00 | 43.92 | O |
| ATOM | 1901 | O | HOH | W | 182 | 61.724 | 4.743 | −4.943 | 1.00 | 40.15 | O |
| ATOM | 1902 | O | HOH | W | 183 | 45.766 | −13.602 | −17.558 | 1.00 | 43.68 | O |
| ATOM | 1903 | O | HOH | W | 184 | 22.884 | 2.561 | 4.926 | 1.00 | 47.13 | O |
| ATOM | 1904 | O | HOH | W | 185 | 48.916 | −1.933 | 17.364 | 1.00 | 42.62 | O |
| ATOM | 1905 | O | HOH | W | 186 | 60.901 | 7.496 | −0.807 | 1.00 | 41.90 | O |
| ATOM | 1906 | O | HOH | W | 187 | 35.182 | −6.469 | 18.698 | 1.00 | 43.82 | O |
| ATOM | 1907 | O | HOH | W | 188 | 46.945 | −12.091 | 6.847 | 1.00 | 52.01 | O |
| ATOM | 1908 | O | HOH | W | 189 | 49.168 | 13.025 | 13.668 | 1.00 | 55.54 | O |
| ATOM | 1909 | O | HOH | W | 190 | 56.439 | 4.770 | 17.676 | 1.00 | 48.84 | O |
| ATOM | 1910 | O | HOH | W | 191 | 32.573 | −2.472 | 13.145 | 1.00 | 40.86 | O |
| ATOM | 1911 | O | HOH | W | 192 | 58.947 | 10.978 | −7.704 | 1.00 | 44.19 | O |
| ATOM | 1912 | O | HOH | W | 193 | 53.652 | 15.680 | 7.067 | 1.00 | 41.53 | O |
| ATOM | 1913 | O | HOH | W | 194 | 48.180 | 11.653 | 17.019 | 1.00 | 40.39 | O |
| ATOM | 1914 | O | HOH | W | 195 | 44.089 | −16.191 | 2.616 | 1.00 | 38.39 | O |
| ATOM | 1915 | O | HOH | W | 196 | 38.299 | 11.232 | −6.433 | 1.00 | 41.19 | O |
| ATOM | 1916 | O | HOH | W | 197 | 24.841 | −11.274 | 5.659 | 1.00 | 42.30 | O |

TABLE 3

```
Employed sequence numbering scheme for human
neutrophil elastase (following Navia MA et al.,
1989, Proc. Natl. Acad. Sci. USA 86: 7-11)
  16 17 18 19 20 21 22 23 24 25 26 27
ILE VAL GLY GLY ARG ARG ALA ARG PRO HIS ALA TRP
  28 29 30 31 32 33 34 35 36 38 39 40
PRO PHE MET VAL SER LEU GLN LEU ARG GLY GLY HIS
  41 42 43 44 45 46 47 48 49 50 51 52
PHE CYS GLY ALA THR LEU ILE ALA PRO ASN PHE VAL
  53 54 55 56 57 58 59 60 61 62 62A 62B
MET SER ALA ALA HIS CYS VAL ALA ASN VAL ASN VAL
  63 64 65 65A 66 67 68 69 70 71 72 73
ARG ALA VAL ARG VAL VAL LEU GLY ALA HIS ASN LEU
  74 75 76 77 78 79 80 81 82 83 84 85
SER ARG ARG GLU PRO THR ARG GLN VAL PHE ALA VAL
  86 87 88 89 90 91 92 94 95 98 99 99A
GLN ARG ILE PHE GLU ASP GLY TYR ASP PRO VAL ASN
99B 100 101 102 103 104 105 106 107 108 109 110
LEU LEU ASN ASP ILE VAL ILE LEU GLN LEU ASN GLY
111 112 113 114 115 116 117 118 119 120 121 122
SER ALA THR ILE ASN ALA ASN VAL GLN VAL ALA GLN
123 124 125 126 127 128 129 130 131 132 133 134
LEU PRO ALA GLN GLY ARG ARG LEU GLY ASN GLY VAL
135 136 137 138 139 140 141 142 143 144 145 147
GLN CYS LEU ALA MET GLY TRP GLY LEU LEU GLY ARG
148 149 150 151 152 153 154 155 156 157 158 159
ASN ARG GLY ILE ALA SER VAL LEU GLN GLU LEU ASN
160 162 163 164 165 166 167 168 177 178 179 180
VAL THR VAL VAL THR SER LEU CYS ARG ARG SER ASN
181 182 183 184 185 186 186A 187 188 188A 189 190
VAL CYS THR LEU VAL ARG GLY ARG GLN ALA CLY VAL
191 192 193 194 195 196 197 198 199 200 201 204
CYS PHE GLY ASP SER GLY SER PRO LEU VAL CYS ASN
205 208 209 210 211 212 213 214 215 216 217 218
GLY LEU ILE HIS GLY ILE ALA SER PHE VAL ARG GLY
219 220 221 222 222A 223 224 225 226 227 228 229
GLY CYS ALA SER GLY LEU TYR PRO ASP ALA PHE ALA
230 231 232 233 234 235 236 237 238 239 240 241
PRO VAL ALA GLN PHE VAL ASN TRP ILE ASP SER ILE
242 243
ILE GLN
```

TABLE 4
Data collection and Final Refinement Statistics for Crystals of Apo
Human Neutrophil Elastase
Table 4a: Data Collection Statistics for Apo Human Neutrophil
Elastase

| | |
|---|---|
| Total number of observations | 1599060 |
| Number of unique reflections | 42465 |
| Completeness [%]¹⁾ | |
| overall | 99.4 (41-1.86 Å) |
| highest resolution shell | 95.8 (1.96-1.86 Å) |
| R_{merge} [%]²⁾ | |
| overall | 12.0 (41-1.86 Å) |
| highest resolution shell | 38.6 (1.96-1.86 Å) |

¹⁾Ratio of the number of possible and the number of present uniques
reflections
²⁾R_{merge} = ΣₕΣᵢ|I_{(h,i)} − <I₍ₕ₎>|/ΣₕI_{(h,i)} where I_{(h,i)} is the intensity
value of the i-th measurement of h and <I₍ₕ₎> is the corresponding mean
value of h for all i measurements of h. The summation is over
all measurements

Table 4b: Final Refinement Statistics for Apo Human Neutrophil
Elastase

| | |
|---|---|
| Resolution range [Å] | 41-1.86 |
| Number of unique reflections | 42465 |
| Molecules per asymmetric unit | 2 |
| Total number of atoms (excl. H) | 3917 |
| Solvent atoms | 481 |
| R-factor¹⁾ | |
| overall | 0.172 (0.216) |
| highest resolution shell²⁾ | 0.246 (0.268) |
| Average B-factors [Å²] | 16.2 |
| RMS deviations from target values | |
| bond lengths [Å] | 0.016 |
| Bond angles [°] | 1.8 |

¹⁾R = Σₕₖₗ||Fₒ| − |F_{c}||/Σₕₖₗ|Fₒ|, value for R_{free} given in brackcts
²⁾1.96-1.86 Å

TABLE 5
Data collection and Final Refinement Statistics for Crystals of
Human Neutrophil Elastase Inhibited with Inhibitor I
Table 5a: Data Collection Statistics for Human Neutrophil Elastase
Inhibited with Inhibitor I

| | |
|---|---|
| Total number of observations | 396270 |
| Number of unique reflections | 14498 |
| Completeness [%]¹⁾ | |
| overall | 97.2 (35-2.0 Å) |
| highest resolution shell | 79.7 (2.07-2.0 Å) |
| R_{merge} [%]²⁾ | |
| overall | 9.2 (35-2.0 Å) |
| highest resolution shell | 34.8 (2.07-2.0 Å) |

¹⁾Ratio of the number of possible and the number of present uniques
reflections
²⁾R_{merge} = ΣₕΣᵢ|I_{(h,i)} − <I₍ₕ₎>|/ΣₕI_{(h,i)} where I_{(h,i)} is the intensity value
of the i-th measurement of h and <I₍ₕ₎> is the corresponding mean value
of h for all i measurements of h. The summation is over all measurements

Table 5b: Final Refinement Statistics for Human Neutrophil Elastase
Inhibited with Inhibitor I

| | |
|---|---|
| Resolution range [Å] | 35-2.0 |
| Number of unique reflections | 14498 |
| Molecules per asymmetric unit | 1 |
| Total number of atoms (excl. H) | 1916 |
| Solvent atoms | 197 |
| R-factor¹⁾ | |
| overall | 0.159 (0.224) |
| highest resolution shell²⁾ | 0.215 (0.285) |
| Average B-factors [Å²] | 22.3 |
| RMS deviations from target values | |
| bond lengths [Å] | 0.017 |
| Bond angles [°] | 1.9 |

¹⁾R = Σₕₖₗ||Fₒ| − |F_{c}||/Σₕₖₗ|Fₒ|, value for R_{free} given in brackets
²⁾2.07-2.0 Å

1. A crystalline form of human neutrophil elastase (HNE) wherein said HNE comprises the sequence given in Table 3.

2. A crystalline form of HNE according to claim 1 wherein said crystalline form has one or more of the following characteristics:
(i) space group P4₁2₁2,
(ii) unit cell dimensions of a=b=123 Å±1°, c=69 Å±1%, α=β=γ=90°,
(iii) two molecules in the asymmetric unit,
(iv) a calculated solvent content of 43%±5%, and
(v) a tetragonal crystal system.

3. A crystalline form of HNE according to claim 1 wherein said HNE is in an un-inhibited form and has a three-dimensional structure characterized by the atomic x,y,z-coordinates set out in Table 1 of this specification or a derivative set as expressed in any reference frame.

4. A crystalline form of HNE according to claim 1 wherein the said HNE is inhibited by a compound of formula I and has a three-dimensional structure characterized by the atomic x,y,z-coordinates set out in Table 2 of this specification or a derivative set as expressed in any reference frame.

5. A crystalline form of HNE according to claim 1 wherein the said HNE is inhibited by a compound of formula II and has a three-dimensional structure characterized by x,y,z-coordinates of the set of atoms of the amino acid residues His57, Tyr94, Pro98, Leu99B, Asp102, Val190, Cys191, Phe 192, Asp194, Ser195, Ala213, Ser214, Phe215, and Val216, and wherein these atomic x,y,z-coordinates are within a root mean square deviation of not more than 1.0 Å of the x,y,z-coordinates of the atoms in the set of the said amino acid residues His57, Tyr94, Pro98, Leu99B, Asp102, Val190, Cys191, Phe 192, Asp194, Ser195, Ala213, Ser214, Phe215, and Val216 listed in Table 2.

6. A crystalline form of HNE according to claim 1 wherein the enzyme is in un-inhibited form (“apo enzyme”).

7. A crystalline form of HNE according to claim 6 wherein said un-inhibited HNE has a three-dimensional structure characterized by the atomic x,y,z-coordinates set out in Table 1 of this specification or a derivative set as expressed in any reference frame.

8. A crystalline form of HNE according to or claim 6 wherein said crystalline form has one or more of the following characteristics:
(i) space group P4₁2₁2,
(ii) unit cell dimensions of a=b=123 Å±1°, c=69 Å±1%, α=β=γ=90°,
(iii) two molecules in the asymmetric unit,
(iv) a calculated solvent content of 43%±5%, and
(v) a tetragonal crystal system.

9. A crystalline form of HNE according to claim 8 wherein said un-inhibited HNE has a three-dimensional structure characterized by the atomic x,y,z-coordinates set out in Table 1 of this specification or a derivative set as expressed in any reference frame.

10. A crystalline form of HNE according to claim 1 wherein the enzyme is in inhibited form (“ligand complexed enzyme”) and wherein the ligand is a compound of formula II.

11. A crystalline form of HNE according to claim 10 wherein said crystalline form has one or more of the following characteristics:
(i) space group P6₃,
(ii) unit cell dimensions of a=b=74 Å±1°, c=71 Å±1%, α=β=90°, γ=120°,
(iii) one molecules in the asymmetric unit,
(iv) a calculated solvent content of 52%±5%, and
(v) a hexagonal crystal system.

12. A crystalline form of HNE according to claim 10 wherein the said HNE is inhibited by a compound of formula II and has a three-dimensional structure characterized by x,y,z-coordinates of the set of atoms of the amino acid residues His57, Tyr94, Pro98, Leu99B, Asp102, Val190, Cys191, Phe 192, Asp194, Ser195, Ala213, Ser214, Phe215, and Val216, and wherein these atomic x,y,z-coordinates are within a root mean square deviation of not more than 1.0 Å of the x,y,z-coordinates of the atoms in the set of the said amino acid residues His57, Tyr94, Pro98, Leu99B, Asp102, Val190, Cys191, Phe 192, Asp194, Ser195, Ala213, Ser214, Phe215, and Val216 listed in Table 2.

13. A crystalline form of HNE according to claim 1 wherein the ligand is a compound of formula I.

14. A crystalline form of HNE according to claim 13 wherein the said HNE is inhibited by a compound of formula I and has a three-dimensional structure characterized by the atomic x,y,z-coordinates set out in Table 2 of this specification or a derivative set as expressed in any reference frame.

15. A crystalline form of HNE according to claim 13 wherein said crystalline form has one or more of the following characteristics:
(i) space group P6₃,
(ii) unit cell dimensions of a=b=74 Å±1°, c=71 Å±1%, α=β=90°, γ=120°,
(iii) one molecules in the asymmetric unit,
(iv) a calculated solvent content of 52%±5%, and
(v) a hexagonal crystal system.

16. A crystalline form of HNE according to claim 15 wherein the said HNE is inhibited by a compound of formula I and has a three-dimensional structure characterized by the atomic x,y,z-coordinates set out in Table 2 of this specification or a derivative set as expressed in any reference frame.

17. A crystalline form according to any one of claims 1-16, wherein the catalytically active domain of human neutrophil elastase comprises a binding site, wherein the binding site is defined by the x.y.z-coordinates of atoms in the set of amino acid residues given by the list: His57, Tyr94, Pro98, Leu99B, Asp102, Val190, Cys191, Phe 192, Asp194, Ser195, Ala213, Ser214, Phe215, Val216 or their equivalent, wherein the atomic coordinates are listed in Tables 1 and 2.

18. A crystalline form according to any one of claims 1, 3, 5, 6, 7, 8, 9, or 12, wherein the conformation of the consecutive loop element Tyr94-Asp95-Pro98-Val99-Asn99A-Leu99B-Leu 100-Asn101 as defined by the x,y,z-coordinates of atoms in the before given set of listed amino acid residues listed in Table 1 is characterized by main chain phi and psi angles of: (−61±4°, 121±4°) for Tyr94, (−108±4°, 104±4°) for Asp95, (−74±4°, −11±4°) for Pro98, (−91±4°, −47±4°) for Val99, (−98±4°, 0±4°) for Asn99A, (44±4°, 61±4°) for Leu99B, (−102±4°, 147±4°) for Leu100, and (53±4°, 50±4°) for Asn101.

19. A crystalline form according to any one of claims 1, 4, 5, 10, 12, 13, 14, 15, or 16 wherein the conformation of the consecutive loop element Tyr94-Asp95-Pro98-Val99-Asn99A-Leu99B-Leu100-Asn101 as defined by the x,y,z-coordinates of atoms in the before given set of listed amino acid residues listed in Table 2 is characterized by main chain phi and psi angles of: (−35±4°, 118±4°) for Tyr94, (−124±4°, 90±4°) for Asp95, (−61±4°, −30±4°) for Pro98, (−75±4°, −34±4°) for Val99, (−92±4°, −1±4°) for Asn99A, (58±4°, 43±4°) for Leu99B, (−115±4°, 153±4°) for Leu100, and (60±4°, 46±4°) for Asn101.

20. A chemical entity which modulates the catalytic activity of HNE wherein said modulator is a compound of formula II, wherein the binding of said compound of formula II to HNE as defined by its atomic x,y,z-coordinates and the x,y,z-coordinates of the set of atoms of the amino acid residues His57, Tyr94, Pro98, Leu99B, Asp102, Val190, Cys191, Phe 192, Asp194, Ser195, Ala213, Ser214, Phe215, and Val216 is characterised by one or more of the following characteristics:
(i) a m-R4-phenyl moiety placed in the S1 subsite of the active site binding pocket of human neutrophil elastase, inserting its m-R4 moiety and its aromatic ring system between the lining side chains of Val190, Phe 192, Asp194, Ser195, Ala213, Val 216 and the lining main chain elements of Cys191-Phe192 and of Phe215;
(ii) a central pyrimidinone ring lying above the side chain of His57 and the main chain element Ser214-Val216;
(iii) a p-R3-phenyl moiety placed in the S2 subsite of the active site binding pocket of human neutrophil elastase, inserting its p-R3 moiety and its aromatic ring system between the lining side chains of His57, Tyr94, Pro98, Leu99B, Asp102, Ser214, and Phe215;
(iv) a relative orientation of the p-R3-phenyl moiety to the central pyrimidinone ring as defined by a N3-C4-C13-C14 torsion angle of 120±10°;
(v) a relative orientation of the m-R4-phenyl moiety to the central pyrimidinone ring as defined by a C2-N1-C7-C12 torsion angle of −80±10°;
(vi) m-R4-phenyl ring centroid distances of 5.45±0.15 Å to Ser195 Cβ, 5.37±0.15 Å to Phe192 Cβ, 4.69±0.15 Å to Val 215 Cβ, and 6.93±0.15 Å to Ala213 Cβ;
(vii) pyrimidinone ring centroid distances of 4.63±0.15 Å to Phe215 Cβ, 6.02±0.15 Å to His57 Cβ, and 7.67±0.15 Å to Phe192 Cβ;
(viii) p-R3-phenyl ring centroid distances of 4.08±0.15 Å to His57 Cβ, 4.50±0.15 Å to Phe215 Cβ, 5.63±0.15 Å to Leu99B Cβ, 5.73±0.15 Å to Pro98 Cα, 9.39±0.15 Å to Tyr94 Cβ, and 6.41±0.15 Å to Asp102 Cβ;
(ix) m-R4-phenyl ring atom distances of 4.37±0.15 Å for C11-Val216 Cβ, 4.79±0.15 Å for C12-Val216 Cβ, 4.70±0.15 Å for C9-Ser195 Cβ, and 4.74±0.14 Å for C8-Ser195 Cβ;
(x) pyrimidinone ring atom distances of 3.79±0.15 Å for C2-Phe215 Cβ, 4.10±0.15 Å for N3-Phe 215 Cβ, 6.70±0.15 Å for C5-Phe192 Cβ, and 7.80±0.15 Å for C6-Phe192 Cβ;
(xi) a pyrimidinone O1-Val216 main chain N distance of 3.23±0.15 Å; and
(xii) p-R3-phenyl ring atom distances of 5.14±0.15 Å for C18-Leu99B Cγ, 4.45±0.15 Å for C17-Leu99B Cγ, 4.19±0.15 Å for C14-His57 Cβ, and 3.91±0.15 Å for His57 Cβ.

21. A Chemical entity which modulates the catalytic activity of HNE wherein said modulator is a compound of formula I, wherein the binding of said compound of formula I to HNE as defined by its atomic x,y,z-coordinates and the x,y,z-coordinates of the set of atoms of the amino acid residues His57, Tyr94, Pro98, Leu99B, Asp102, Val190, Cys191, Phe 192, Asp194, Ser195, Ala213, Ser214, Phe215, and Val216 listed in Table 2 is characterised by one or more of the following characteristics:
(i) a m-trifluoromethyl-phenyl moiety placed in the S1 subsite of the active site binding pocket of human neutrophil elastase, inserting its m-trifluoromethyl-phenyl moiety and its aromatic ring system between the lining side chains of Val190, Phe 192, Asp194, Ser195, Ala213, Val 216 and the lining main chain elements of Cys191-Phe192 and of Phe215;
(ii) a central pyrimidinone ring lying above the side chain of His57 and the main chain element Ser214-Val216;
(iii) a p-cyano-phenyl moiety placed in the S2 subsite of the active site binding pocket of human neutrophil elastase, inserting its p-cyano moiety and its aromatic ring system between the lining side chains of His57, Tyr94, Pro98, Leu99B, Asp102, Ser214, and Phe215;
(iv) a relative orientation of the p-cyano-phenyl moiety to the central pyrimidinone ring as defined by a N3-C4-C13-C14 torsion angle of 120±10°;
(v) a relative orientation of the m-trifluoromethyl-phenyl moiety to the central pyrimidinone ring as defined by a C2-N1-C7-C12 torsion angle of −80±10°;
(vi) m-trifluoromethyl-phenyl ring centroid distances of 5.45±0.15 Å to Ser195 Cβ, 5.37±0.15 Å to Phe192 Cβ, 4.69±0.15 Å to Val 215 Cβ, and 6.93±0.15 Å to Ala213 Cβ;
(vii) pyrimidinone ring centroid distances of 4.63±0.15 Å to Phe215 Cβ, 6.02±0.15 Å to His57 Cβ, and 7.67±0.15 Å to Phe192 Cβ;
(viii) p-cyano-phenyl ring centroid distances of 4.08±0.15 Å to His57 Cβ, 4.50±0.15 Å to Phe215 Cβ, 5.63±0.15 Å to Leu99B Cβ, 5.73±0.15 Å to Pro98 Cα, 9.39±0.15 Å to Tyr94 Cβ, and 6.41±0.15 Å to Asp102 Cβ;
(ix) m-trifluoromethyl-phenyl ring atom distances of 4.37±0.15 Å for C11-Val216 Cβ, 4.79±0.15 Å for C12-Val216 Cβ, 4.70±0.15 Å for C9-Ser195 Cβ, and 4.74±0.14 Å for C8-Ser195 Cβ;
(x) pyrimidinone ring atom distances of 3.79±0.15 Å for C2-Phe215 Cβ, 4.10±0.15 Å for N3-Phe 215 Cβ, 6.70±0.15 Å for C5-Phe192 Cβ, and 7.80±0.15 Å for C6-Phe192 Cβ;
(xi) a pyrimidinone O1-Val216 main chain N distance of 3.23±0.15 Å; and
(xii) p-cyano-phenyl ring atom distances of 5.14±0.15 Å for C18-Leu99B Cγ, 4.45±0.15 Å for C17-Leu99B Cγ, 4.19±0.15 Å for C14-His57 Cβ, and 3.91±0.15 Å for His57 Cβ.

22. A method of designing a human neutrophil elastase modulator, comprising the step of using the atomic coordinates of a crystalline form of human neutrophil elastase as defined in any one of claims 1 to 19.

23. A method of selecting a human neutrophil elastase modulator, comprising the step of using the atomic coordinates of a crystalline form of human neutrophil elastase as defined in any one of claims 1 to 19.

24. A method of designing or selecting a human neutrophil elastase modulator comprising the steps of (a) exploring the atomic coordinates of human neutrophil elastase in Table 1 and Table 2 for information on the three-dimensional characteristics of the protein surface; and (b) selecting or designing a human neutrophil elastase modulator using the active site binding pocket information.

25. A method of designing the three-dimensional structure of a second crystal form of human neutrophil elastase comprising the step of applying difference Fourier or molecular replacement methods using the atomic coordinates of an original crystal as presented in table 1 and Table 2 to model the structure of the crystal second form, wherein the active site binding pocket of the second crystal form is equivalent to that in the original crystal.

26. A method of producing a modulator of human neutrophil elastase comprising identifying a compound or molecule or designing a compound or molecule that fits into the active site binding pocket of human neutrophil elastase in its un-inhibited conformation, wherein said un-inhibited conformation of the active site binding pocket of human neutrophil elastase is defined by the x,y,z-coordinates of atoms in the set of amino acid residues given by the list His57, Tyr94, Pro98, Leu99B, Asp 102, Val190, Cys191, Phe 192, Asp194, Ser195, Ala213, Ser214, Phe215, and Val216, the atomic coordinates being listed in Table 1, thereby producing a modulator of human neutrophil elastase.

27. A method of producing a modulator of human neutrophil elastase comprising identifying a compound or molecule or designing a compound or molecule that fits into the active site binding pocket of human neutrophil elastase in its inhibitor I inhibited conformation, wherein said inhibitor I inhibited conformation of the active site binding pocket of human neutrophil elastase is defined by the x,y,z-coordinates of atoms in the set of amino acid residues given by the list His57, Tyr94, Pro98, Leu99B, Asp102, Val190, Cys191, Phe 192, Asp194, Ser195, Ala213, Ser214, Phe215, and Val216, the atomic coordinates being listed in Table 2, thereby producing a modulator of human neutrophil elastase.
